SEQUENCE LISTING (1 sequence)

SEQ ID NO: 1 - protein, 361 aa, Homo Sapiens
MEYMPMEGGGGSKVTTVVATPGQGPDRPQEVSYTDTKVIGNGSFGVVYQAKLCDSGELVAIKKVLQDKRFKNRELQIMRKLDHCNIVRLRYFFYSSGEKKDEVYLNLVLDYVPETVYRVARHYSRAKQTLPVIYVKLYMYQLFRSLAYIHSFGICHRDIKPQNLLLDPDTAVLKLCDFGSAKQLVRGEPNVSYICSRYYRAPELIFGATDYTSSIDVWSAGCVLAELLLGQPIFPGDSGVDQLVEIIKVLGTPTREQIREMNPNYTEFKFPQIKAHPWTKVFRPRTPPEAIALCSRLLEYTPTARLTPLEACAHSFFDELRDPNVKLPNGRDTPALFNFTTQELSSNPPLATILIPPHARI

(19) United States
(12) Patent Application Publication
(10) Pub. No.: US 2008/0004433 A1
(43) Pub. Date: Jan. 3, 2008

(54) Characterization of the GSK-3beta protein and methods of use thereof

(76) Inventors: Dirksen E. Bussiere, San Leandro, CA (US); Min He, Union City, CA (US); Vincent P. Le, San Francisco, CA (US); Johanna M. Jansen, San Francisco, CA (US); S. Michael Chin, Albany, CA (US); Eric Martin, El Cerrito, CA (US)

Correspondence Address: CHIRON CORPORATION;INTELLECTUAL PROPERTY - R440, PO BOX 8097, EMERYVILLE, CA, 94662-8097, US

(21) Appl. No.: 11/879,719

(22) Filed: Jul. 18, 2007

Related U.S. Application Data
(63) Continuation of application No. 10/450,422, filed on Dec. 11, 2003, now abandoned.
(60) Provisional application No. 60/233,538, filed on Sep. 19, 2000.

(51) Int. Cl. C07K 14/00; C07K 1/00; G06G 7/48

(52) U.S. Cl. 530/402; 703/11

(57) ABSTRACT

The invention provides the three-dimensional structure of a construct of glycogen synthase kinase 3 (GSK3); crystals of a construct of glycogen synthase kinase 3-β (GSK3-β) containing the protein's catalytic kinase domain; a method for crystallizing the protein construct to provide a GSK3 crystal sufficient for structure determination; and a method for using the GSK3 construct's three-dimensional structure for the identification of possible therapeutic compounds in the treatment of various disease conditions mediated by GSK3 activity.

### CROSS-REFERENCES TO RELATED APPLICATIONS

This application is a continuation of U.S. patent application Ser. No. 10/450,422, which is the National Stage of International Application No. PCT/US01/29549, filed Sep. 19, 2001, which claims the benefit of U.S. Provisional Application No. 60/233,538, filed Sep. 19, 2000. Each application is incorporated herein by reference in its entirety.

### FIELD OF THE INVENTION

This invention relates to the three-dimensional structure of glycogen synthase kinase 3 (GSK3), to crystals of a construct of GSK3 SEQ ID NO:1, to methods for forming crystals of the GSK3 construct SEQ ID NO:1, to methods for determining the crystal structure of the GSK3 construct SEQ ID NO:1, and to methods for using the three-dimensional structure of GSK3 to identify possible therapeutic compounds for the treatment of various disease conditions mediated by GSK3 activity.

### BACKGROUND OF THE INVENTION

Glycogen synthase kinase 3 (GSK3) is a serine/threonine kinase for which two isoforms, α and β, have been identified. Woodgett, Trends Biochem. Sci., 16:177-81 (1991). Both GSK3 isoforms are constitutively active in resting cells. GSK3 was originally identified as a kinase that inhibits glycogen synthase by direct phosphorylation. Upon insulin activation, GSK3 is inactivated, thereby allowing the activation of glycogen synthase and possibly other insulin-dependent events, such as glucose transport. Subsequently, it has been shown that GSK3 activity is also inactivated by other growth factors that, like insulin, signal through receptor tyrosine kinases (RTKs). Examples of such signaling molecules include IGF-1 and EGF. Saito et al., Biochem. J., 303:27-31 (1994); Welsh et al., Biochem. J. 294:625-29 (1993); and Cross et al., Biochem. J., 303:21-26 (1994).

Agents that inhibit GSK3 activity are useful in the treatment of disorders that are mediated by GSK3 activity. In addition, inhibition of GSK3 mimics the activation of growth factor signaling pathways and consequently GSK3 inhibitors are useful in the treatment of diseases in which such pathways are insufficiently active. Examples of diseases that can be treated with GSK3 inhibitors include diabetes, Alzheimer's disease, CNS disorders such as bipolar disorder, and immune potentiation-related conditions, among others.

Because inhibitors of GSK3 are useful in the treatment of many diseases, the identification of new inhibitors of GSK3 would be highly desirable. The present invention provides a method for identifying possible therapeutic compounds for the treatment of various disease conditions mediated by GSK3 activity. The method of the present invention utilizes the three-dimensional structure of a GSK3 construct SEQ ID NO:1 that contains the protein's catalytic domain to identify possible therapeutic compounds and to optimize the structure of lead therapeutic compounds.

### SUMMARY OF THE INVENTION

In accordance with the present invention, the three-dimensional structure of a construct of glycogen synthase kinase 3 (GSK3) SEQ ID NO:1 is provided.

In one aspect, the invention provides crystals of a construct of glycogen synthase kinase 3-β (GSK3-β) SEQ ID NO:1 containing the protein's catalytic kinase domain.

In another aspect of the invention, a method for crystallizing the protein construct to provide a GSK3 crystal sufficient for structure determination is provided.

In a further aspect, the three-dimensional structure of the GSK3 construct SEQ ID NO:1 is provided.

In yet another aspect, a method is provided for using the GSK3 (SEQ ID NO:1) construct's three-dimensional structure for the identification of possible therapeutic compounds in the treatment of various disease conditions mediated by GSK3 activity.

### BRIEF DESCRIPTION OF THE DRAWINGS

The foregoing aspects and many of the attendant advantages of this invention will become more readily appreciated by reference to the following detailed description, when taken in conjunction with the accompanying drawings, wherein:

FIG. 1 is an illustration of the structure of the GSK3-β construct SEQ ID NO:1;

FIG. 2 is an illustration of the structure of the GSK3-β construct SEQ ID NO:1 active site;

FIG. 3 is a flow diagram of a representative method of the invention using the three-dimensional structure of the GSK3-β construct SEQ ID NO:1 for identifying possible therapeutic compounds for mediating GSK3-β activity; and

FIG. 4 is a flow diagram of a representative method of the invention using the three-dimensional structure of the GSK3-β construct SEQ ID NO:1 for identifying possible therapeutic compounds for mediating GSK3-β activity.

### DETAILED DESCRIPTION OF THE PREFERRED EMBODIMENT

In accordance with the present invention, crystals of a protein construct of glycogen synthase kinase 3-β (GSK3-β) SEQ ID NO:1 containing the protein's catalytic kinase domain, methods for crystallizing the protein construct, the three-dimensional structure of the protein construct, and methods for using the three-dimensional structure for the identification of possible therapeutic compounds in the treatment of various disease conditions mediated by GSK3-β activity are provided.

### The GSK3-β Protein Construct: Expression, Purification, and Crystallization

In one aspect, the invention provides a composition that includes a GSK3-β construct SEQ ID NO:1 that contains the protein's catalytic kinase domain. The construct includes at least residues 37-384 of human GSK3-β and lacks the 36 amino acids at the protein's C-terminus. The composition is a crystalline form sufficient for structure determination by diffraction studies by X-ray.

It will be appreciated that GSK3 protein constructs other than the construct described herein, for example, active mutants or variants thereof, can provide three-dimensional structural information useful in identifying possible therapeutic compounds in the treatment of various disease conditions mediated by GSK3 activity.

Construct Sequence. The construct sequence, SEQ ID NO: 1, is provided below. The asterisk indicates the first residue that is seen in the crystal structure. The following construct and additional useful constructs and their preparation are described in co-pending U.S. Patent Application Ser. No. 60/221,242, filed Jul. 27, 2000, the disclosure of which is incorporated herein by reference in its entirety and for all purposes.

(SEQ ID NO: 1)

N-terminus: MEYMPMEGGGGSK
*VTTVVATPGQGPDRPQEVSYTDTKVIGNGSFGVVYQAKLCDSGEL
VAIKKVLQDKRFKNRELQIMRKLDHCNIVRLRYFFYSSGEKKDEVYLNLV
LDYVPETVYRVARHYSRAKQTLPVIYVKLYMYQLFRSLAYIHSFGICHRD
IKPQNLLLDPDTAVLKLCDFGSAKQLVRGEPNVSYICSRYYRAPELIFGA
TDYTSSIDVWSAGCVLAELLLGQPIFPGDSGVDQLVEIIKVLGTPTREQI
REMNPNYTEFKFPQIKAHPWTKVFRPRTPPEAIALCSRLLEYTPTARLTP
LEACAHSFFDELRDPNVKLPNGRDTPALFNFTTQELSSNPPLATILIPPH
ARI: C-terminus

Construct Purification. The GSK3-β protein construct SEQ ID NO:1 was extracted from SF-9 cells infected with a baculovirus carrying GSK3-β 580 cDNA construct. The GSK3-β protein construct SEQ ID NO:1 was purified to apparent homogeneity using S-Fractogel, Phenyl-650 M, and Glu-tag affinity chromatographies. The purified protein was then concentrated for crystallization. Purification of the construct is described in Example 1.

Construct Crystallization. Protein crystals can be formed from solutions of the GSK3 construct SEQ ID NO:1 by, for example, the hanging drop technique. A representative method for forming suitable crystals of the GSK3 construct SEQ ID NO:1 suitable for structure determination is described in Example 2.

It will be appreciated that various crystallization methods including, for example, microcrystallization methods can be utilized to obtain three-dimensional structural information useful in identifying possible therapeutic compounds in the treatment of various disease conditions mediated by GSK3 activity.

### The GSK3-β Protein Construct Structure

In another aspect of the invention, the three-dimensional structure of the GSK3 protein construct SEQ ID NO:1 is provided. Amino acid sequence data and atomic coordinates derived from X-ray diffraction data were used to determine the construct's three-dimensional structure. The construct's atomic coordinates were calculated from an electron density map produced from the combination of X-ray diffraction and phase data.

With the GSK3 construct SEQ ID NO:1 available in crystalline form suitable for structural determination, the crystal structure can be obtained by a variety of techniques. In a representative method, diffraction patterns were obtained using an X-ray image plate device. Phase data was then obtained by a combination of molecular replacement and cross-crystal averaging techniques. Electron density maps were then constructed and the structure solved and molecule built. The resulting structure was refined and the structure validated. The ultimate result was an atomic model of the GSK3 construct SEQ ID NO:1. A representative method for obtaining the GSK3 crystal structure is described in Example 3.

It will be appreciated that the GSK3 structure can be solved by a variety of methods.

The statistics for collecting the crystallographic data are summarized in Table 1.

TABLE 1
Data and Model Statistics for Structure Solution

| | Native 2.2 Å |
|---|---|
| Space Group | P222(1) |
| Highest Resolution (Å) | 2.2 |
| R_{merge} (%) | 10.4 |
| I/σ (total) | 13.7 |
| Final Shell | 2.26 Å-2.20 Å |
| I/Iσ (Final Shell) | 3.3 |
| R-factor (%) | 26.2 |
| free-R factor (%) | 30.8 |

The three-dimensional structure of the GSK3-β construct SEQ ID NO:1 provided as a tabulation of atomic coordinates is given in Table 2. In the table, “OH2” and “GOL” refer to structural water and glycerol molecules, and “TER” refers to the terminus of a peptide chain.

TABLE 2
GSK3-β Construct Atomic Coordinates

| | | | | | | | | | | |
|---|---|---|---|---|---|---|---|---|---|---|
| ATOM | 1 | CB | VAL | A | 37 | 80.033 | 24.831 | −19.608 | 1.00 | 50.57 |
| ATOM | 2 | CG1 | VAL | A | 37 | 81.501 | 25.233 | −19.582 | 1.00 | 50.37 |
| ATOM | 3 | CG2 | VAL | A | 37 | 79.466 | 24.947 | −21.017 | 1.00 | 51.61 |
| ATOM | 4 | C | VAL | A | 37 | 80.398 | 23.336 | −17.638 | 1.00 | 50.20 |
| ATOM | 5 | O | VAL | A | 37 | 79.972 | 24.095 | −16.767 | 1.00 | 51.05 |
| ATOM | 6 | N | VAL | A | 37 | 78.411 | 22.987 | −19.067 | 1.00 | 49.79 |
| ATOM | 7 | CA | VAL | A | 37 | 79.846 | 23.399 | −19.056 | 1.00 | 50.62 |
| ATOM | 8 | N | THR | A | 38 | 81.331 | 22.418 | −17.401 | 1.00 | 49.02 |
| ATOM | 9 | CA | THR | A | 38 | 81.900 | 22.262 | −16.068 | 1.00 | 48.04 |
| ATOM | 10 | CB | THR | A | 38 | 81.970 | 20.789 | −15.652 | 1.00 | 48.38 |
| ATOM | 11 | OG1 | THR | A | 38 | 83.312 | 20.328 | −15.604 | 1.00 | 49.77 |
| ATOM | 12 | CG2 | THR | A | 38 | 81.167 | 19.848 | −16.525 | 1.00 | 47.77 |
| ATOM | 13 | C | THR | A | 38 | 83.262 | 22.918 | −15.914 | 1.00 | 47.01 |
| ATOM | 14 | O | THR | A | 38 | 84.143 | 22.760 | −16.755 | 1.00 | 48.09 |
| ATOM | 15 | N | THR | A | 39 | 83.436 | 23.626 | −14.806 | 1.00 | 45.08 |
| ATOM | 16 | CA | THR | A | 39 | 84.701 | 24.275 | −14.508 | 1.00 | 42.86 |
| ATOM | 17 | CB | THR | A | 39 | 84.538 | 25.790 | −14.267 | 1.00 | 43.86 |
| ATOM | 18 | OG1 | THR | A | 39 | 84.623 | 26.130 | −12.895 | 1.00 | 44.44 |
| ATOM | 19 | CG2 | THR | A | 39 | 83.258 | 26.382 | −14.821 | 1.00 | 43.65 |
| ATOM | 20 | C | THR | A | 39 | 85.395 | 23.571 | −13.338 | 1.00 | 40.74 |
| ATOM | 21 | O | THR | A | 39 | 84.759 | 23.220 | −12.334 | 1.00 | 40.93 |
| ATOM | 22 | N | VAL | A | 40 | 86.694 | 23.349 | −13.485 | 1.00 | 38.40 |
| ATOM | 23 | CA | VAL | A | 40 | 87.483 | 22.679 | −12.464 | 1.00 | 35.30 |
| ATOM | 24 | CB | VAL | A | 40 | 88.180 | 21.427 | −13.027 | 1.00 | 34.48 |
| ATOM | 25 | CG1 | VAL | A | 40 | 89.077 | 20.788 | −11.983 | 1.00 | 34.49 |
| ATOM | 26 | CG2 | VAL | A | 40 | 87.142 | 20.429 | −13.516 | 1.00 | 34.61 |
| ATOM | 27 | C | VAL | A | 40 | 88.513 | 23.636 | −11.873 | 1.00 | 34.74 |
| ATOM | 28 | O | VAL | A | 40 | 89.329 | 24.220 | −12.601 | 1.00 | 33.31 |
| ATOM | 29 | N | VAL | A | 41 | 88.454 | 23.806 | −10.543 | 1.00 | 33.24 |
| ATOM | 30 | CA | VAL | A | 41 | 89.362 | 24.707 | −9.838 | 1.00 | 30.39 |
| ATOM | 31 | CB | VAL | A | 41 | 88.589 | 25.899 | −9.223 | 1.00 | 31.41 |
| ATOM | 32 | CG1 | VAL | A | 41 | 87.840 | 26.681 | −10.294 | 1.00 | 28.06 |
| ATOM | 33 | CG2 | VAL | A | 41 | 87.632 | 25.422 | −8.136 | 1.00 | 29.89 |
| ATOM | 34 | C | VAL | A | 41 | 90.122 | 23.990 | −8.723 | 1.00 | 29.47 |
| ATOM | 35 | O | VAL | A | 41 | 89.751 | 22.891 | −8.311 | 1.00 | 28.80 |
| ATOM | 36 | N | ALA | A | 42 | 91.167 | 24.638 | −8.219 | 1.00 | 28.13 |
| ATOM | 37 | CA | ALA | A | 42 | 91.960 | 24.092 | −7.119 | 1.00 | 29.60 |
| ATOM | 38 | CB | ALA | A | 42 | 93.450 | 24.229 | −7.400 | 1.00 | 28.11 |
| ATOM | 39 | C | ALA | A | 42 | 91.581 | 24.837 | −5.835 | 1.00 | 29.66 |
| ATOM | 40 | O | ALA | A | 42 | 91.963 | 25.990 | −5.648 | 1.00 | 28.27 |
| ATOM | 41 | N | THR | A | 43 | 90.789 | 24.196 | −4.981 | 1.00 | 30.99 |
| ATOM | 42 | CA | THR | A | 43 | 90.337 | 24.849 | −3.762 | 1.00 | 34.40 |
| ATOM | 43 | CB | THR | A | 43 | 88.911 | 25.332 | −3.955 | 1.00 | 36.00 |
| ATOM | 44 | OG1 | THR | A | 43 | 88.371 | 25.782 | −2.727 | 1.00 | 43.97 |
| ATOM | 45 | CG2 | THR | A | 43 | 87.985 | 24.275 | −4.510 | 1.00 | 37.41 |
| ATOM | 46 | C | THR | A | 43 | 90.408 | 23.974 | −2.506 | 1.00 | 33.11 |
| ATOM | 47 | O | THR | A | 43 | 90.083 | 22.791 | −2.538 | 1.00 | 33.54 |
| ATOM | 48 | N | PRO | A | 44 | 90.826 | 24.572 | −1.375 | 1.00 | 32.56 |
| ATOM | 49 | CD | PRO | A | 44 | 91.239 | 25.963 | −1.245 | 1.00 | 35.07 |
| ATOM | 50 | CA | PRO | A | 44 | 90.930 | 23.899 | −0.087 | 1.00 | 33.94 |
| ATOM | 51 | CB | PRO | A | 44 | 92.056 | 24.682 | 0.621 | 1.00 | 33.35 |
| ATOM | 52 | CG | PRO | A | 44 | 92.363 | 25.860 | −0.251 | 1.00 | 33.23 |
| ATOM | 53 | C | PRO | A | 44 | 89.646 | 24.069 | 0.709 | 1.00 | 32.73 |
| ATOM | 54 | O | PRO | A | 44 | 88.967 | 25.089 | 0.591 | 1.00 | 31.50 |
| ATOM | 55 | N | GLY | A | 45 | 89.324 | 23.087 | 1.532 | 1.00 | 35.79 |
| ATOM | 56 | CA | GLY | A | 45 | 88.123 | 23.201 | 2.342 | 1.00 | 39.32 |
| ATOM | 57 | C | GLY | A | 45 | 88.377 | 24.052 | 3.576 | 1.00 | 41.20 |
| ATOM | 58 | O | GLY | A | 45 | 89.460 | 24.624 | 3.724 | 1.00 | 38.49 |
| ATOM | 59 | N | GLN | A | 46 | 87.399 | 24.114 | 4.483 | 1.00 | 46.21 |
| ATOM | 60 | CA | GLN | A | 46 | 87.570 | 24.868 | 5.729 | 1.00 | 48.58 |
| ATOM | 61 | CB | GLN | A | 46 | 86.280 | 25.586 | 6.143 | 1.00 | 50.56 |
| ATOM | 62 | CG | GLN | A | 46 | 85.157 | 24.661 | 6.612 | 1.00 | 53.97 |
| ATOM | 63 | CD | GLN | A | 46 | 84.699 | 24.926 | 8.044 | 1.00 | 54.45 |
| ATOM | 64 | OE1 | GLN | A | 46 | 85.143 | 25.874 | 8.699 | 1.00 | 54.99 |
| ATOM | 65 | NE2 | GLN | A | 46 | 83.801 | 24.083 | 8.535 | 1.00 | 54.65 |
| ATOM | 66 | C | GLN | A | 46 | 88.035 | 23.908 | 6.822 | 1.00 | 49.85 |
| ATOM | 67 | O | GLN | A | 46 | 87.531 | 23.929 | 7.948 | 1.00 | 50.43 |
| ATOM | 68 | N | GLY | A | 47 | 88.992 | 23.042 | 6.465 | 1.00 | 50.37 |
| ATOM | 69 | CA | GLY | A | 47 | 89.492 | 22.066 | 7.410 | 1.00 | 51.27 |
| ATOM | 70 | C | GLY | A | 47 | 91.005 | 21.903 | 7.413 | 1.00 | 51.26 |
| ATOM | 71 | O | GLY | A | 47 | 91.731 | 22.809 | 7.831 | 1.00 | 51.04 |
| ATOM | 72 | N | PRO | A | 48 | 91.504 | 20.724 | 6.987 | 1.00 | 51.25 |
| ATOM | 73 | CD | PRO | A | 48 | 90.694 | 19.579 | 6.520 | 1.00 | 52.23 |
| ATOM | 74 | CA | PRO | A | 48 | 92.939 | 20.413 | 6.984 | 1.00 | 51.17 |
| ATOM | 75 | CB | PRO | A | 48 | 92.949 | 18.883 | 6.954 | 1.00 | 51.98 |
| ATOM | 76 | CG | PRO | A | 48 | 91.722 | 18.526 | 6.185 | 1.00 | 52.45 |
| ATOM | 77 | C | PRO | A | 48 | 93.703 | 20.964 | 5.785 | 1.00 | 50.33 |
| ATOM | 78 | O | PRO | A | 48 | 94.648 | 20.331 | 5.310 | 1.00 | 51.68 |
| ATOM | 79 | N | ASP | A | 49 | 93.312 | 22.141 | 5.305 | 1.00 | 48.80 |
| ATOM | 80 | CA | ASP | A | 49 | 93.991 | 22.755 | 4.165 | 1.00 | 48.33 |
| ATOM | 81 | CB | ASP | A | 49 | 95.381 | 23.259 | 4.595 | 1.00 | 48.73 |
| ATOM | 82 | CG | ASP | A | 49 | 95.853 | 24.465 | 3.805 | 1.00 | 49.64 |
| ATOM | 83 | OD1 | ASP | A | 49 | 96.434 | 24.267 | 2.722 | 1.00 | 51.08 |
| ATOM | 84 | OD2 | ASP | A | 49 | 95.649 | 25.606 | 4.272 | 1.00 | 49.48 |
| ATOM | 85 | C | ASP | A | 49 | 94.069 | 21.761 | 2.984 | 1.00 | 46.42 |
| ATOM | 86 | O | ASP | A | 49 | 93.071 | 21.119 | 2.667 | 1.00 | 47.58 |
| ATOM | 87 | N | ARG | A | 50 | 95.245 | 21.636 | 2.353 | 1.00 | 43.75 |
| ATOM | 88 | CA | ARG | A | 50 | 95.458 | 20.721 | 1.214 | 1.00 | 40.37 |
| ATOM | 89 | CB | ARG | A | 50 | 95.508 | 19.239 | 1.659 | 1.00 | 41.78 |
| ATOM | 90 | CG | ARG | A | 50 | 94.176 | 18.621 | 2.058 | 1.00 | 44.71 |
| ATOM | 91 | CD | ARG | A | 50 | 93.970 | 17.261 | 1.398 | 1.00 | 47.92 |
| ATOM | 92 | NE | ARG | A | 50 | 93.230 | 16.337 | 2.265 | 1.00 | 51.36 |
| ATOM | 93 | CZ | ARG | A | 50 | 91.918 | 16.436 | 2.519 | 1.00 | 52.40 |
| ATOM | 94 | NH1 | ARG | A | 50 | 91.181 | 17.377 | 1.923 | 1.00 | 53.58 |
| ATOM | 95 | NH2 | ARG | A | 50 | 91.343 | 15.589 | 3.368 | 1.00 | 52.49 |
| ATOM | 96 | C | ARG | A | 50 | 94.437 | 20.938 | 0.092 | 1.00 | 36.07 |
| ATOM | 97 | O | ARG | A | 50 | 93.374 | 20.329 | 0.054 | 1.00 | 34.88 |
| ATOM | 98 | N | PRO | A | 51 | 94.760 | 21.823 | −0.848 | 1.00 | 33.81 |
| ATOM | 99 | CD | PRO | A | 51 | 96.002 | 22.606 | −0.891 | 1.00 | 34.21 |
| ATOM | 100 | CA | PRO | A | 51 | 93.879 | 22.123 | −1.975 | 1.00 | 32.21 |
| ATOM | 101 | CB | PRO | A | 51 | 94.686 | 23.116 | −2.814 | 1.00 | 31.98 |
| ATOM | 102 | CG | PRO | A | 51 | 95.678 | 23.697 | −1.870 | 1.00 | 32.93 |
| ATOM | 103 | C | PRO | A | 51 | 93.569 | 20.878 | −2.797 | 1.00 | 30.79 |
| ATOM | 104 | O | PRO | A | 51 | 94.436 | 20.049 | −3.040 | 1.00 | 32.41 |
| ATOM | 105 | N | GLN | A | 52 | 92.327 | 20.768 | −3.227 | 1.00 | 31.01 |
| ATOM | 106 | CA | GLN | A | 52 | 91.882 | 19.649 | −4.038 | 1.00 | 31.10 |
| ATOM | 107 | CB | GLN | A | 52 | 91.036 | 18.656 | −3.228 | 1.00 | 30.92 |
| ATOM | 108 | CG | GLN | A | 52 | 89.656 | 19.164 | −2.825 | 1.00 | 34.68 |
| ATOM | 109 | CD | GLN | A | 52 | 89.132 | 18.495 | −1.556 | 1.00 | 37.20 |
| ATOM | 110 | OE1 | GLN | A | 52 | 88.074 | 17.858 | −1.561 | 1.00 | 38.16 |
| ATOM | 111 | NE2 | GLN | A | 52 | 89.874 | 18.637 | −0.461 | 1.00 | 37.13 |
| ATOM | 112 | C | GLN | A | 52 | 91.108 | 20.158 | −5.251 | 1.00 | 30.15 |
| ATOM | 113 | O | GLN | A | 52 | 90.525 | 21.245 | −5.221 | 1.00 | 29.33 |
| ATOM | 114 | N | GLU | A | 53 | 91.099 | 19.367 | −6.312 | 1.00 | 30.79 |
| ATOM | 115 | CA | GLU | A | 53 | 90.383 | 19.740 | −7.518 | 1.00 | 30.71 |
| ATOM | 116 | CB | GLU | A | 53 | 90.837 | 18.877 | −8.694 | 1.00 | 31.87 |
| ATOM | 117 | CG | GLU | A | 53 | 91.680 | 19.628 | −9.717 | 1.00 | 33.95 |
| ATOM | 118 | CD | GLU | A | 53 | 92.675 | 18.733 | −10.440 | 1.00 | 35.69 |
| ATOM | 119 | OE1 | GLU | A | 53 | 93.828 | 19.181 | −10.661 | 1.00 | 35.06 |
| ATOM | 120 | OE2 | GLU | A | 53 | 92.304 | 17.584 | −10.781 | 1.00 | 35.68 |
| ATOM | 121 | C | GLU | A | 53 | 88.880 | 19.615 | −7.291 | 1.00 | 29.25 |
| ATOM | 122 | O | GLU | A | 53 | 88.400 | 18.567 | −6.846 | 1.00 | 30.67 |
| ATOM | 123 | N | VAL | A | 54 | 88.148 | 20.697 | −7.578 | 1.00 | 28.00 |
| ATOM | 124 | CA | VAL | A | 54 | 86.695 | 20.739 | −7.398 | 1.00 | 27.10 |
| ATOM | 125 | CB | VAL | A | 54 | 86.281 | 21.745 | −6.292 | 1.00 | 26.17 |
| ATOM | 126 | CG1 | VAL | A | 54 | 84.771 | 21.910 | −6.253 | 1.00 | 23.81 |
| ATOM | 127 | CG2 | VAL | A | 54 | 86.805 | 21.294 | −4.937 | 1.00 | 22.59 |
| ATOM | 128 | C | VAL | A | 54 | 86.001 | 21.113 | −8.705 | 1.00 | 27.97 |
| ATOM | 129 | O | VAL | A | 54 | 86.364 | 22.089 | −9.359 | 1.00 | 28.40 |
| ATOM | 130 | N | SER | A | 55 | 85.009 | 20.324 | −9.082 | 1.00 | 27.75 |
| ATOM | 131 | CA | SER | A | 55 | 84.281 | 20.564 | −10.317 | 1.00 | 29.06 |
| ATOM | 132 | CB | SER | A | 55 | 84.321 | 19.317 | −11.212 | 1.00 | 27.82 |
| ATOM | 133 | OG | SER | A | 55 | 85.649 | 18.850 | −11.348 | 1.00 | 29.80 |
| ATOM | 134 | C | SER | A | 55 | 82.845 | 20.965 | −10.063 | 1.00 | 28.17 |
| ATOM | 135 | O | SER | A | 55 | 82.125 | 20.291 | −9.322 | 1.00 | 28.82 |
| ATOM | 136 | N | TYR | A | 56 | 82.434 | 22.052 | −10.710 | 1.00 | 28.14 |
| ATOM | 137 | CA | TYR | A | 56 | 81.073 | 22.558 | −10.607 | 1.00 | 29.61 |
| ATOM | 138 | CB | TYR | A | 56 | 80.969 | 23.720 | −9.605 | 1.00 | 27.30 |
| ATOM | 139 | CG | TYR | A | 56 | 81.822 | 24.941 | −9.904 | 1.00 | 25.16 |
| ATOM | 140 | CD1 | TYR | A | 56 | 83.132 | 25.026 | −9.443 | 1.00 | 25.56 |
| ATOM | 141 | CE1 | TYR | A | 56 | 83.905 | 26.154 | −9.682 | 1.00 | 26.15 |
| ATOM | 142 | CD2 | TYR | A | 56 | 81.308 | 26.021 | −10.612 | 1.00 | 23.48 |
| ATOM | 143 | CE2 | TYR | A | 56 | 82.073 | 27.149 | −10.859 | 1.00 | 23.57 |
| ATOM | 144 | CZ | TYR | A | 56 | 83.370 | 27.210 | −10.391 | 1.00 | 24.23 |
| ATOM | 145 | OH | TYR | A | 56 | 84.140 | 28.328 | −10.636 | 1.00 | 27.89 |
| ATOM | 146 | C | TYR | A | 56 | 80.562 | 22.994 | −11.972 | 1.00 | 31.23 |
| ATOM | 147 | O | TYR | A | 56 | 81.354 | 23.278 | −12.878 | 1.00 | 30.17 |
| ATOM | 148 | N | THR | A | 57 | 79.243 | 23.057 | −12.107 | 1.00 | 33.31 |
| ATOM | 149 | CA | THR | A | 57 | 78.620 | 23.476 | −13.356 | 1.00 | 35.43 |
| ATOM | 150 | CB | THR | A | 57 | 78.412 | 22.276 | −14.296 | 1.00 | 35.27 |
| ATOM | 151 | OG1 | THR | A | 57 | 77.984 | 22.698 | −15.583 | 1.00 | 37.54 |
| ATOM | 152 | CG2 | THR | A | 57 | 77.433 | 21.244 | −13.785 | 1.00 | 33.54 |
| ATOM | 153 | C | THR | A | 57 | 77.310 | 24.215 | −13.087 | 1.00 | 36.61 |
| ATOM | 154 | O | THR | A | 57 | 76.936 | 24.426 | −11.929 | 1.00 | 36.38 |
| ATOM | 155 | N | ASP | A | 58 | 76.619 | 24.603 | −14.163 | 1.00 | 36.33 |
| ATOM | 156 | CA | ASP | A | 58 | 75.347 | 25.315 | −14.064 | 1.00 | 36.49 |
| ATOM | 157 | CB | ASP | A | 58 | 74.297 | 24.467 | −13.332 | 1.00 | 39.46 |
| ATOM | 158 | CG | ASP | A | 58 | 73.794 | 23.286 | −14.157 | 1.00 | 41.91 |
| ATOM | 159 | OD1 | ASP | A | 58 | 73.025 | 22.461 | −13.607 | 1.00 | 42.29 |
| ATOM | 160 | OD2 | ASP | A | 58 | 74.166 | 23.182 | −15.352 | 1.00 | 44.17 |
| ATOM | 161 | C | ASP | A | 58 | 75.513 | 26.673 | −13.391 | 1.00 | 35.84 |
| ATOM | 162 | O | ASP | A | 58 | 74.643 | 27.125 | −12.655 | 1.00 | 34.21 |
| ATOM | 163 | N | THR | A | 59 | 76.639 | 27.312 | −13.665 | 1.00 | 35.88 |
| ATOM | 164 | CA | THR | A | 59 | 76.965 | 28.615 | −13.114 | 1.00 | 37.28 |
| ATOM | 165 | CB | THR | A | 59 | 78.403 | 28.955 | −13.476 | 1.00 | 36.95 |
| ATOM | 166 | OG1 | THR | A | 59 | 79.257 | 27.884 | −13.110 | 1.00 | 37.20 |
| ATOM | 167 | CG2 | THR | A | 59 | 78.930 | 30.221 | −12.831 | 1.00 | 38.01 |
| ATOM | 168 | C | THR | A | 59 | 76.009 | 29.703 | −13.596 | 1.00 | 38.01 |
| ATOM | 169 | O | THR | A | 59 | 75.641 | 29.736 | −14.767 | 1.00 | 39.28 |
| ATOM | 170 | N | LYS | A | 60 | 75.613 | 30.597 | −12.674 | 1.00 | 37.94 |
| ATOM | 171 | CA | LYS | A | 60 | 74.704 | 31.698 | −13.000 | 1.00 | 36.62 |
| ATOM | 172 | CB | LYS | A | 60 | 73.259 | 31.196 | −13.139 | 1.00 | 37.83 |
| ATOM | 173 | CG | LYS | A | 60 | 72.517 | 31.015 | −11.822 | 1.00 | 38.44 |
| ATOM | 174 | CD | LYS | A | 60 | 71.034 | 31.300 | −11.975 | 1.00 | 38.40 |
| ATOM | 175 | CE | LYS | A | 60 | 70.199 | 30.076 | −11.645 | 1.00 | 39.94 |
| ATOM | 176 | NZ | LYS | A | 60 | 68.909 | 30.441 | −10.992 | 1.00 | 41.86 |
| ATOM | 177 | C | LYS | A | 60 | 74.789 | 32.812 | −11.957 | 1.00 | 36.32 |
| ATOM | 178 | O | LYS | A | 60 | 74.913 | 32.543 | −10.768 | 1.00 | 36.41 |
| ATOM | 179 | N | VAL | A | 61 | 74.735 | 34.060 | −12.421 | 1.00 | 34.99 |
| ATOM | 180 | CA | VAL | A | 61 | 74.815 | 35.226 | −11.555 | 1.00 | 33.46 |
| ATOM | 181 | CB | VAL | A | 61 | 75.084 | 36.508 | −12.368 | 1.00 | 34.41 |
| ATOM | 182 | CG1 | VAL | A | 61 | 74.892 | 37.749 | −11.503 | 1.00 | 34.88 |
| ATOM | 183 | CG2 | VAL | A | 61 | 76.488 | 36.473 | −12.970 | 1.00 | 33.02 |
| ATOM | 184 | C | VAL | A | 61 | 73.536 | 35.386 | −10.742 | 1.00 | 33.94 |
| ATOM | 185 | O | VAL | A | 61 | 72.431 | 35.331 | −11.295 | 1.00 | 31.92 |
| ATOM | 186 | N | ILE | A | 62 | 73.692 | 35.554 | −9.413 | 1.00 | 30.70 |
| ATOM | 187 | CA | ILE | A | 62 | 72.534 | 35.686 | −8.518 | 1.00 | 30.06 |
| ATOM | 188 | CB | ILE | A | 62 | 72.322 | 34.424 | −7.649 | 1.00 | 31.30 |
| ATOM | 189 | CG2 | ILE | A | 62 | 71.972 | 33.218 | −8.511 | 1.00 | 29.51 |
| ATOM | 190 | CG1 | ILE | A | 62 | 73.554 | 34.141 | −6.777 | 1.00 | 29.23 |
| ATOM | 191 | CD1 | ILE | A | 62 | 73.268 | 33.219 | −5.610 | 1.00 | 31.27 |
| ATOM | 192 | C | ILE | A | 62 | 72.615 | 36.903 | −7.602 | 1.00 | 28.13 |
| ATOM | 193 | O | ILE | A | 62 | 71.629 | 37.255 | −6.966 | 1.00 | 29.85 |
| ATOM | 194 | N | GLY | A | 63 | 73.773 | 37.546 | −7.539 | 1.00 | 26.58 |
| ATOM | 195 | CA | GLY | A | 63 | 73.909 | 38.715 | −6.696 | 1.00 | 26.14 |
| ATOM | 196 | C | GLY | A | 63 | 75.217 | 39.436 | −6.899 | 1.00 | 27.79 |
| ATOM | 197 | O | GLY | A | 63 | 76.092 | 38.967 | −7.627 | 1.00 | 29.41 |
| ATOM | 198 | N | ASN | A | 64 | 75.364 | 40.578 | −6.249 | 1.00 | 27.39 |
| ATOM | 199 | CA | ASN | A | 64 | 76.589 | 41.353 | −6.362 | 1.00 | 28.10 |
| ATOM | 200 | CB | ASN | A | 64 | 76.724 | 41.953 | −7.779 | 1.00 | 28.83 |
| ATOM | 201 | CG | ASN | A | 64 | 75.706 | 43.040 | −8.063 | 1.00 | 29.90 |
| ATOM | 202 | OD1 | ASN | A | 64 | 74.819 | 43.302 | −7.253 | 1.00 | 32.34 |
| ATOM | 203 | ND2 | ASN | A | 64 | 75.829 | 43.689 | −9.220 | 1.00 | 31.39 |
| ATOM | 204 | C | ASN | A | 64 | 76.656 | 42.451 | −5.300 | 1.00 | 26.72 |
| ATOM | 205 | O | ASN | A | 64 | 75.726 | 42.639 | −4.527 | 1.00 | 27.02 |
| ATOM | 206 | N | GLY | A | 65 | 77.759 | 43.177 | −5.306 | 1.00 | 25.86 |
| ATOM | 207 | CA | GLY | A | 65 | 77.975 | 44.267 | −4.395 | 1.00 | 24.32 |
| ATOM | 208 | C | GLY | A | 65 | 79.267 | 44.955 | −4.715 | 1.00 | 24.77 |
| ATOM | 209 | O | GLY | A | 65 | 79.871 | 44.661 | −5.747 | 1.00 | 22.63 |
| ATOM | 210 | N | SER | A | 66 | 79.714 | 45.861 | −3.838 | 1.00 | 23.92 |
| ATOM | 211 | CA | SER | A | 66 | 80.968 | 46.573 | −4.069 | 1.00 | 23.93 |
| ATOM | 212 | CB | SER | A | 66 | 81.127 | 47.755 | −3.102 | 1.00 | 28.01 |
| ATOM | 213 | OG | SER | A | 66 | 81.468 | 47.312 | −1.792 | 1.00 | 33.78 |
| ATOM | 214 | C | SER | A | 66 | 82.174 | 45.651 | −4.006 | 1.00 | 21.75 |
| ATOM | 215 | O | SER | A | 66 | 83.293 | 46.050 | −4.340 | 1.00 | 23.12 |
| ATOM | 216 | N | PHE | A | 67 | 81.948 | 44.419 | −3.576 | 1.00 | 21.22 |
| ATOM | 217 | CA | PHE | A | 67 | 83.019 | 43.426 | −3.459 | 1.00 | 21.95 |
| ATOM | 218 | CB | PHE | A | 67 | 82.635 | 42.390 | −2.397 | 1.00 | 19.80 |
| ATOM | 219 | CG | PHE | A | 67 | 81.371 | 41.638 | −2.727 | 1.00 | 21.25 |
| ATOM | 220 | CD1 | PHE | A | 67 | 80.178 | 41.958 | −2.107 | 1.00 | 21.15 |
| ATOM | 221 | CD2 | PHE | A | 67 | 81.375 | 40.618 | −3.677 | 1.00 | 21.87 |
| ATOM | 222 | CE1 | PHE | A | 67 | 79.014 | 41.285 | −2.418 | 1.00 | 18.94 |
| ATOM | 223 | CE2 | PHE | A | 67 | 80.217 | 39.940 | −3.992 | 1.00 | 19.41 |
| ATOM | 224 | CZ | PHE | A | 67 | 79.036 | 40.274 | −3.361 | 1.00 | 21.96 |
| ATOM | 225 | C | PHE | A | 67 | 83.241 | 42.684 | −4.788 | 1.00 | 21.21 |
| ATOM | 226 | O | PHE | A | 67 | 84.356 | 42.288 | −5.117 | 1.00 | 21.27 |
| ATOM | 227 | N | GLY | A | 68 | 82.154 | 42.459 | −5.504 | 1.00 | 21.10 |
| ATOM | 228 | CA | GLY | A | 68 | 82.218 | 41.729 | −6.744 | 1.00 | 19.70 |
| ATOM | 229 | C | GLY | A | 68 | 80.882 | 41.159 | −7.110 | 1.00 | 17.87 |
| ATOM | 230 | O | GLY | A | 68 | 79.899 | 41.874 | −7.159 | 1.00 | 21.48 |
| ATOM | 231 | N | VAL | A | 69 | 80.841 | 39.876 | −7.410 | 1.00 | 19.22 |
| ATOM | 232 | CA | VAL | A | 69 | 79.590 | 39.262 | −7.845 | 1.00 | 18.99 |
| ATOM | 233 | CB | VAL | A | 69 | 79.686 | 39.054 | −9.385 | 1.00 | 20.73 |
| ATOM | 234 | CG1 | VAL | A | 69 | 78.464 | 38.338 | −9.938 | 1.00 | 19.08 |
| ATOM | 235 | CG2 | VAL | A | 69 | 79.904 | 40.398 | −10.058 | 1.00 | 20.67 |
| ATOM | 236 | C | VAL | A | 69 | 79.371 | 37.914 | −7.214 | 1.00 | 16.65 |
| ATOM | 237 | O | VAL | A | 69 | 80.301 | 37.263 | −6.861 | 1.00 | 17.62 |
| ATOM | 238 | N | VAL | A | 70 | 78.144 | 37.477 | −7.124 | 1.00 | 17.29 |
| ATOM | 239 | CA | VAL | A | 70 | 77.877 | 36.162 | −6.558 | 1.00 | 19.87 |
| ATOM | 240 | CB | VAL | A | 70 | 76.944 | 36.225 | −5.328 | 1.00 | 19.21 |
| ATOM | 241 | CG1 | VAL | A | 70 | 76.827 | 34.839 | −4.701 | 1.00 | 16.63 |
| ATOM | 242 | CG2 | VAL | A | 70 | 77.469 | 37.240 | −4.317 | 1.00 | 18.33 |
| ATOM | 243 | C | VAL | A | 70 | 77.286 | 35.217 | −7.591 | 1.00 | 21.82 |
| ATOM | 244 | O | VAL | A | 70 | 76.320 | 35.551 | −8.255 | 1.00 | 22.52 |
| ATOM | 245 | N | TYR | A | 71 | 77.875 | 34.032 | −7.705 | 1.00 | 25.04 |
| ATOM | 246 | CA | TYR | A | 71 | 77.407 | 33.030 | −8.657 | 1.00 | 28.92 |
| ATOM | 247 | CB | TYR | A | 71 | 78.603 | 32.453 | −9.446 | 1.00 | 29.97 |
| ATOM | 248 | CG | TYR | A | 71 | 79.251 | 33.358 | −10.469 | 1.00 | 33.56 |
| ATOM | 249 | CD1 | TYR | A | 71 | 78.711 | 33.511 | −11.742 | 1.00 | 35.58 |
| ATOM | 250 | CE1 | TYR | A | 71 | 79.326 | 34.312 | −12.693 | 1.00 | 36.15 |
| ATOM | 251 | CD2 | TYR | A | 71 | 80.426 | 34.033 | −10.173 | 1.00 | 34.86 |
| ATOM | 252 | CE2 | TYR | A | 71 | 81.045 | 34.843 | −11.111 | 1.00 | 37.15 |
| ATOM | 253 | CZ | TYR | A | 71 | 80.492 | 34.978 | −12.372 | 1.00 | 37.44 |
| ATOM | 254 | OH | TYR | A | 71 | 81.117 | 35.771 | −13.313 | 1.00 | 39.50 |
| ATOM | 255 | C | TYR | A | 71 | 76.746 | 31.838 | −7.974 | 1.00 | 29.85 |
| ATOM | 256 | O | TYR | A | 71 | 77.210 | 31.348 | −6.942 | 1.00 | 29.76 |
| ATOM | 257 | N | GLN | A | 72 | 75.736 | 31.294 | −8.627 | 1.00 | 30.90 |
| ATOM | 258 | CA | GLN | A | 72 | 75.121 | 30.075 | −8.154 | 1.00 | 31.38 |
| ATOM | 259 | CB | GLN | A | 72 | 73.616 | 30.050 | −8.379 | 1.00 | 33.05 |
| ATOM | 260 | CG | GLN | A | 72 | 72.915 | 28.963 | −7.572 | 1.00 | 34.55 |
| ATOM | 261 | CD | GLN | A | 72 | 71.721 | 28.374 | −8.285 | 1.00 | 36.84 |
| ATOM | 262 | OE1 | GLN | A | 72 | 71.837 | 27.379 | −9.001 | 1.00 | 37.72 |
| ATOM | 263 | NE2 | GLN | A | 72 | 70.558 | 28.988 | −8.092 | 1.00 | 39.46 |
| ATOM | 264 | C | GLN | A | 72 | 75.792 | 28.950 | −8.931 | 1.00 | 32.33 |
| ATOM | 265 | O | GLN | A | 72 | 76.249 | 29.163 | −10.068 | 1.00 | 33.82 |
| ATOM | 266 | N | ALA | A | 73 | 75.896 | 27.783 | −8.332 | 1.00 | 31.53 |
| ATOM | 267 | CA | ALA | A | 73 | 76.551 | 26.678 | −8.998 | 1.00 | 31.65 |
| ATOM | 268 | CB | ALA | A | 73 | 78.061 | 26.826 | −8.914 | 1.00 | 31.06 |
| ATOM | 269 | C | ALA | A | 73 | 76.127 | 25.346 | −8.426 | 1.00 | 32.36 |
| ATOM | 270 | O | ALA | A | 73 | 75.386 | 25.272 | −7.447 | 1.00 | 31.34 |
| ATOM | 271 | N | LYS | A | 74 | 76.618 | 24.295 | −9.048 | 1.00 | 33.73 |
| ATOM | 272 | CA | LYS | A | 74 | 76.324 | 22.956 | −8.622 | 1.00 | 35.30 |
| ATOM | 273 | CB | LYS | A | 74 | 75.177 | 22.372 | −9.443 | 1.00 | 36.50 |
| ATOM | 274 | CG | LYS | A | 74 | 74.794 | 20.960 | −9.037 | 1.00 | 37.82 |
| ATOM | 275 | CD | LYS | A | 74 | 73.610 | 20.453 | −9.843 | 1.00 | 40.81 |
| ATOM | 276 | CE | LYS | A | 74 | 74.036 | 19.415 | −10.869 | 1.00 | 43.32 |
| ATOM | 277 | NZ | LYS | A | 74 | 74.647 | 20.042 | −12.078 | 1.00 | 46.77 |
| ATOM | 278 | C | LYS | A | 74 | 77.559 | 22.098 | −8.760 | 1.00 | 36.85 |
| ATOM | 279 | O | LYS | A | 74 | 78.124 | 21.975 | −9.855 | 1.00 | 37.48 |
| ATOM | 280 | N | LEU | A | 75 | 77.973 | 21.496 | −7.648 | 1.00 | 37.35 |
| ATOM | 281 | CA | LEU | A | 75 | 79.131 | 20.622 | −7.655 | 1.00 | 37.20 |
| ATOM | 282 | CB | LEU | A | 75 | 79.586 | 20.322 | −6.227 | 1.00 | 34.69 |
| ATOM | 283 | CG | LEU | A | 75 | 79.445 | 21.467 | −5.222 | 1.00 | 34.55 |
| ATOM | 284 | CD1 | LEU | A | 75 | 79.241 | 20.922 | −3.816 | 1.00 | 32.89 |
| ATOM | 285 | CD2 | LEU | A | 75 | 80.659 | 22.386 | −5.276 | 1.00 | 31.32 |
| ATOM | 286 | C | LEU | A | 75 | 78.747 | 19.333 | −8.370 | 1.00 | 39.29 |
| ATOM | 287 | O | LEU | A | 75 | 77.626 | 18.845 | −8.213 | 1.00 | 38.83 |
| ATOM | 288 | N | CYS | A | 76 | 79.658 | 18.802 | −9.174 | 1.00 | 41.06 |
| ATOM | 289 | CA | CYS | A | 76 | 79.385 | 17.581 | −9.928 | 1.00 | 43.45 |
| ATOM | 290 | CB | CYS | A | 76 | 80.363 | 17.462 | −11.094 | 1.00 | 42.71 |
| ATOM | 291 | SG | CYS | A | 76 | 80.578 | 19.008 | −12.012 | 1.00 | 42.33 |
| ATOM | 292 | C | CYS | A | 76 | 79.442 | 16.345 | −9.035 | 1.00 | 45.25 |
| ATOM | 293 | O | CYS | A | 76 | 78.682 | 15.392 | −9.218 | 1.00 | 46.06 |
| ATOM | 294 | N | ASP | A | 77 | 80.348 | 16.377 | −8.072 | 1.00 | 47.16 |
| ATOM | 295 | CA | ASP | A | 77 | 80.549 | 15.284 | −7.128 | 1.00 | 49.51 |
| ATOM | 296 | CB | ASP | A | 77 | 81.638 | 15.664 | −6.111 | 1.00 | 52.47 |
| ATOM | 297 | CG | ASP | A | 77 | 81.672 | 17.160 | −5.800 | 1.00 | 54.95 |
| ATOM | 298 | OD1 | ASP | A | 77 | 80.869 | 17.607 | −4.944 | 1.00 | 56.19 |
| ATOM | 299 | OD2 | ASP | A | 77 | 82.499 | 17.885 | −6.412 | 1.00 | 54.51 |
| ATOM | 300 | C | ASP | A | 77 | 79.272 | 14.864 | −6.392 | 1.00 | 49.21 |
| ATOM | 301 | O | ASP | A | 77 | 78.893 | 13.693 | −6.415 | 1.00 | 49.89 |
| ATOM | 302 | N | SER | A | 78 | 78.646 | 15.813 | −5.699 | 1.00 | 48.59 |
| ATOM | 303 | CA | SER | A | 78 | 77.449 | 15.536 | −4.899 | 1.00 | 47.33 |
| ATOM | 304 | CB | SER | A | 78 | 77.650 | 16.084 | −3.482 | 1.00 | 47.77 |
| ATOM | 305 | OG | SER | A | 78 | 78.037 | 17.452 | −3.515 | 1.00 | 46.19 |
| ATOM | 306 | C | SER | A | 78 | 76.170 | 16.125 | −5.481 | 1.00 | 47.16 |
| ATOM | 307 | O | SER | A | 78 | 75.074 | 15.833 | −4.994 | 1.00 | 47.02 |
| ATOM | 308 | N | GLY | A | 79 | 76.305 | 16.973 | −6.496 | 1.00 | 46.01 |
| ATOM | 309 | CA | GLY | A | 79 | 75.140 | 17.599 | −7.084 | 1.00 | 43.58 |
| ATOM | 310 | C | GLY | A | 79 | 74.578 | 18.689 | −6.191 | 1.00 | 42.64 |
| ATOM | 311 | O | GLY | A | 79 | 73.542 | 19.271 | −6.495 | 1.00 | 42.66 |
| ATOM | 312 | N | GLU | A | 80 | 75.265 | 18.955 | −5.080 | 1.00 | 41.77 |
| ATOM | 313 | CA | GLU | A | 80 | 74.845 | 19.969 | −4.117 | 1.00 | 40.71 |
| ATOM | 314 | CB | GLU | A | 80 | 75.661 | 19.856 | −2.829 | 1.00 | 41.92 |
| ATOM | 315 | CG | GLU | A | 80 | 75.323 | 18.632 | −1.992 | 1.00 | 44.42 |
| ATOM | 316 | CD | GLU | A | 80 | 76.091 | 18.588 | −0.684 | 1.00 | 47.47 |
| ATOM | 317 | OE1 | GLU | A | 80 | 77.297 | 18.258 | −0.710 | 1.00 | 48.55 |
| ATOM | 318 | OE2 | GLU | A | 80 | 75.488 | 18.896 | 0.372 | 1.00 | 49.38 |
| ATOM | 319 | C | GLU | A | 80 | 74.945 | 21.378 | −4.695 | 1.00 | 39.89 |
| ATOM | 320 | O | GLU | A | 80 | 75.796 | 21.659 | −5.541 | 1.00 | 40.04 |
| ATOM | 321 | N | LEU | A | 81 | 74.055 | 22.253 | −4.237 | 1.00 | 38.33 |
| ATOM | 322 | CA | LEU | A | 81 | 74.010 | 23.632 | −4.701 | 1.00 | 36.18 |
| ATOM | 323 | CB | LEU | A | 81 | 72.566 | 24.127 | −4.728 | 1.00 | 36.89 |
| ATOM | 324 | CG | LEU | A | 81 | 71.763 | 23.803 | −5.984 | 1.00 | 37.28 |
| ATOM | 325 | CD1 | LEU | A | 81 | 71.712 | 22.303 | −6.209 | 1.00 | 38.67 |
| ATOM | 326 | CD2 | LEU | A | 81 | 70.360 | 24.379 | −5.877 | 1.00 | 38.16 |
| ATOM | 327 | C | LEU | A | 81 | 74.840 | 24.542 | −3.812 | 1.00 | 34.75 |
| ATOM | 328 | O | LEU | A | 81 | 74.720 | 24.506 | −2.585 | 1.00 | 36.28 |
| ATOM | 329 | N | VAL | A | 82 | 75.675 | 25.363 | −4.434 | 1.00 | 31.15 |
| ATOM | 330 | CA | VAL | A | 82 | 76.514 | 26.287 | −3.694 | 1.00 | 28.31 |
| ATOM | 331 | CB | VAL | A | 82 | 78.001 | 25.855 | −3.667 | 1.00 | 29.78 |
| ATOM | 332 | CG1 | VAL | A | 82 | 78.211 | 24.602 | −2.825 | 1.00 | 29.34 |
| ATOM | 333 | CG2 | VAL | A | 82 | 78.554 | 25.671 | −5.081 | 1.00 | 30.69 |
| ATOM | 334 | C | VAL | A | 82 | 76.450 | 27.678 | −4.290 | 1.00 | 27.28 |
| ATOM | 335 | O | VAL | A | 82 | 75.925 | 27.892 | −5.400 | 1.00 | 25.63 |
| ATOM | 336 | N | ALA | A | 83 | 77.030 | 28.607 | −3.557 | 1.00 | 22.81 |
| ATOM | 337 | CA | ALA | A | 83 | 77.127 | 29.975 | −3.976 | 1.00 | 20.75 |
| ATOM | 338 | CB | ALA | A | 83 | 76.304 | 30.895 | −3.090 | 1.00 | 19.91 |
| ATOM | 339 | C | ALA | A | 83 | 78.594 | 30.325 | −3.953 | 1.00 | 20.33 |
| ATOM | 340 | O | ALA | A | 83 | 79.320 | 29.867 | −3.068 | 1.00 | 20.70 |
| ATOM | 341 | N | ILE | A | 84 | 79.040 | 31.088 | −4.944 | 1.00 | 19.79 |
| ATOM | 342 | CA | ILE | A | 84 | 80.441 | 31.476 | −5.066 | 1.00 | 19.64 |
| ATOM | 343 | CB | ILE | A | 84 | 81.101 | 30.853 | −6.331 | 1.00 | 19.52 |
| ATOM | 344 | CG2 | ILE | A | 84 | 82.608 | 31.066 | −6.319 | 1.00 | 16.65 |
| ATOM | 345 | CG1 | ILE | A | 84 | 80.758 | 29.358 | −6.423 | 1.00 | 16.97 |
| ATOM | 346 | CD1 | ILE | A | 84 | 81.795 | 28.503 | −7.134 | 1.00 | 21.03 |
| ATOM | 347 | C | ILE | A | 84 | 80.538 | 32.992 | −5.093 | 1.00 | 22.12 |
| ATOM | 348 | O | ILE | A | 84 | 80.055 | 33.621 | −6.031 | 1.00 | 22.68 |
| ATOM | 349 | N | LYS | A | 85 | 81.111 | 33.562 | −4.017 | 1.00 | 22.75 |
| ATOM | 350 | CA | LYS | A | 85 | 81.224 | 35.008 | −3.844 | 1.00 | 24.97 |
| ATOM | 351 | CB | LYS | A | 85 | 81.564 | 35.333 | −2.383 | 1.00 | 25.35 |
| ATOM | 352 | CG | LYS | A | 85 | 81.822 | 36.803 | −2.115 | 1.00 | 25.03 |
| ATOM | 353 | CD | LYS | A | 85 | 80.620 | 37.460 | −1.467 | 1.00 | 27.75 |
| ATOM | 354 | CE | LYS | A | 85 | 80.832 | 37.658 | 0.027 | 1.00 | 28.19 |
| ATOM | 355 | NZ | LYS | A | 85 | 81.048 | 39.089 | 0.363 | 1.00 | 29.61 |
| ATOM | 356 | C | LYS | A | 85 | 82.218 | 35.695 | −4.791 | 1.00 | 27.05 |
| ATOM | 357 | O | LYS | A | 85 | 81.868 | 36.659 | −5.418 | 1.00 | 30.22 |
| ATOM | 358 | N | LYS | A | 86 | 83.445 | 35.243 | −4.901 | 1.00 | 27.41 |
| ATOM | 359 | CA | LYS | A | 86 | 84.405 | 35.914 | −5.825 | 1.00 | 28.29 |
| ATOM | 360 | CB | LYS | A | 86 | 83.963 | 35.770 | −7.294 | 1.00 | 27.88 |
| ATOM | 361 | CG | LYS | A | 86 | 84.896 | 36.486 | −8.266 | 1.00 | 29.66 |
| ATOM | 362 | CD | LYS | A | 86 | 84.710 | 35.997 | −9.700 | 1.00 | 30.66 |
| ATOM | 363 | CE | LYS | A | 86 | 85.545 | 36.801 | −10.683 | 1.00 | 31.77 |
| ATOM | 364 | NZ | LYS | A | 86 | 85.528 | 36.200 | −12.056 | 1.00 | 33.13 |
| ATOM | 365 | C | LYS | A | 86 | 84.583 | 37.403 | −5.528 | 1.00 | 25.91 |
| ATOM | 366 | O | LYS | A | 86 | 83.726 | 38.224 | −5.827 | 1.00 | 25.06 |
| ATOM | 367 | N | VAL | A | 87 | 85.710 | 37.753 | −4.960 | 1.00 | 26.67 |
| ATOM | 368 | CA | VAL | A | 87 | 85.975 | 39.138 | −4.643 | 1.00 | 28.24 |
| ATOM | 369 | CB | VAL | A | 87 | 85.798 | 39.421 | −3.124 | 1.00 | 29.12 |
| ATOM | 370 | CG1 | VAL | A | 87 | 84.605 | 38.644 | −2.574 | 1.00 | 27.52 |
| ATOM | 371 | CG2 | VAL | A | 87 | 87.060 | 39.068 | −2.347 | 1.00 | 29.13 |
| ATOM | 372 | C | VAL | A | 87 | 87.368 | 39.530 | −5.085 | 1.00 | 29.97 |
| ATOM | 373 | O | VAL | A | 87 | 88.314 | 38.771 | −4.931 | 1.00 | 29.26 |
| ATOM | 374 | N | LEU | A | 88 | 87.484 | 40.724 | −5.627 | 1.00 | 33.85 |
| ATOM | 375 | CA | LEU | A | 88 | 88.764 | 41.230 | −6.077 | 1.00 | 37.36 |
| ATOM | 376 | CB | LEU | A | 88 | 88.559 | 42.551 | −6.820 | 1.00 | 36.76 |
| ATOM | 377 | CG | LEU | A | 88 | 89.821 | 43.234 | −7.336 | 1.00 | 37.85 |
| ATOM | 378 | CD1 | LEU | A | 88 | 90.512 | 42.365 | −8.371 | 1.00 | 37.97 |
| ATOM | 379 | CD2 | LEU | A | 88 | 89.490 | 44.606 | −7.899 | 1.00 | 37.34 |
| ATOM | 380 | C | LEU | A | 88 | 89.664 | 41.417 | −4.863 | 1.00 | 40.30 |
| ATOM | 381 | O | LEU | A | 88 | 89.382 | 42.246 | −3.994 | 1.00 | 40.89 |
| ATOM | 382 | N | GLN | A | 89 | 90.717 | 40.606 | −4.784 | 1.00 | 43.74 |
| ATOM | 383 | CA | GLN | A | 89 | 91.636 | 40.638 | −3.653 | 1.00 | 46.09 |
| ATOM | 384 | CB | GLN | A | 89 | 91.929 | 39.211 | −3.181 | 1.00 | 45.39 |
| ATOM | 385 | CG | GLN | A | 89 | 91.367 | 38.863 | −1.809 | 1.00 | 46.90 |
| ATOM | 386 | CD | GLN | A | 89 | 91.570 | 39.957 | −0.778 | 1.00 | 46.72 |
| ATOM | 387 | OE1 | GLN | A | 89 | 90.604 | 40.507 | −0.250 | 1.00 | 47.40 |
| ATOM | 388 | NE2 | GLN | A | 89 | 92.828 | 40.277 | −0.484 | 1.00 | 47.16 |
| ATOM | 389 | C | GLN | A | 89 | 92.949 | 41.333 | −3.983 | 1.00 | 47.93 |
| ATOM | 390 | O | GLN | A | 89 | 93.489 | 41.181 | −5.082 | 1.00 | 49.02 |
| ATOM | 391 | N | ASP | A | 90 | 93.474 | 42.061 | −2.996 | 1.00 | 49.68 |
| ATOM | 392 | CA | ASP | A | 90 | 94.747 | 42.759 | −3.116 | 1.00 | 51.13 |
| ATOM | 393 | CB | ASP | A | 90 | 94.751 | 43.989 | −2.193 | 1.00 | 51.29 |
| ATOM | 394 | CG | ASP | A | 90 | 95.927 | 44.936 | −2.407 | 1.00 | 50.48 |
| ATOM | 395 | OD1 | ASP | A | 90 | 97.031 | 44.464 | −2.756 | 1.00 | 50.57 |
| ATOM | 396 | OD2 | ASP | A | 90 | 95.743 | 46.157 | −2.211 | 1.00 | 50.42 |
| ATOM | 397 | C | ASP | A | 90 | 95.878 | 41.807 | −2.718 | 1.00 | 53.33 |
| ATOM | 398 | O | ASP | A | 90 | 95.963 | 41.394 | −1.555 | 1.00 | 53.28 |
| ATOM | 399 | N | LYS | A | 91 | 96.729 | 41.446 | −3.691 | 1.00 | 54.33 |
| ATOM | 400 | CA | LYS | A | 91 | 97.850 | 40.525 | −3.443 | 1.00 | 55.74 |
| ATOM | 401 | CB | LYS | A | 91 | 98.910 | 40.577 | −4.565 | 1.00 | 56.05 |
| ATOM | 402 | CG | LYS | A | 91 | 98.677 | 41.643 | −5.628 | 1.00 | 56.67 |
| ATOM | 403 | CD | LYS | A | 91 | 99.989 | 42.181 | −6.181 | 1.00 | 56.82 |
| ATOM | 404 | CE | LYS | A | 91 | 99.975 | 43.703 | −6.266 | 1.00 | 56.25 |
| ATOM | 405 | NZ | LYS | A | 91 | 101.277 | 44.307 | −5.849 | 1.00 | 55.53 |
| ATOM | 406 | C | LYS | A | 91 | 98.519 | 40.811 | −2.097 | 1.00 | 56.53 |
| ATOM | 407 | O | LYS | A | 91 | 98.699 | 41.971 | −1.713 | 1.00 | 56.04 |
| ATOM | 408 | N | ARG | A | 92 | 98.870 | 39.734 | −1.389 | 1.00 | 57.95 |
| ATOM | 409 | CA | ARG | A | 92 | 99.509 | 39.812 | −0.070 | 1.00 | 59.85 |
| ATOM | 410 | CB | ARG | A | 92 | 101.031 | 39.971 | −0.194 | 1.00 | 61.28 |
| ATOM | 411 | CG | ARG | A | 92 | 101.765 | 39.884 | 1.149 | 1.00 | 63.11 |
| ATOM | 412 | CD | ARG | A | 92 | 101.170 | 38.809 | 2.066 | 1.00 | 64.33 |
| ATOM | 413 | NE | ARG | A | 92 | 101.534 | 37.450 | 1.636 | 1.00 | 65.78 |
| ATOM | 414 | CZ | ARG | A | 92 | 102.171 | 36.543 | 2.403 | 1.00 | 66.33 |
| ATOM | 415 | NH1 | ARG | A | 92 | 102.468 | 36.805 | 3.681 | 1.00 | 65.70 |
| ATOM | 416 | NH2 | ARG | A | 92 | 102.504 | 35.359 | 1.886 | 1.00 | 66.03 |
| ATOM | 417 | C | ARG | A | 92 | 98.920 | 40.936 | 0.787 | 1.00 | 59.66 |
| ATOM | 418 | O | ARG | A | 92 | 99.505 | 42.015 | 0.933 | 1.00 | 60.23 |
| ATOM | 419 | N | PHE | A | 93 | 97.761 | 40.655 | 1.359 | 1.00 | 59.03 |
| ATOM | 420 | CA | PHE | A | 93 | 97.053 | 41.589 | 2.223 | 1.00 | 57.37 |
| ATOM | 421 | CB | PHE | A | 93 | 96.307 | 42.649 | 1.414 | 1.00 | 58.54 |
| ATOM | 422 | CG | PHE | A | 93 | 96.818 | 44.050 | 1.630 | 1.00 | 59.76 |
| ATOM | 423 | CD1 | PHE | A | 93 | 98.128 | 44.276 | 2.024 | 1.00 | 59.99 |
| ATOM | 424 | CD2 | PHE | A | 93 | 95.989 | 45.141 | 1.436 | 1.00 | 59.95 |
| ATOM | 425 | CE1 | PHE | A | 93 | 98.600 | 45.559 | 2.219 | 1.00 | 60.16 |
| ATOM | 426 | CE2 | PHE | A | 93 | 96.454 | 46.427 | 1.630 | 1.00 | 60.63 |
| ATOM | 427 | CZ | PHE | A | 93 | 97.762 | 46.637 | 2.021 | 1.00 | 60.34 |
| ATOM | 428 | C | PHE | A | 93 | 96.105 | 40.830 | 3.141 | 1.00 | 55.64 |
| ATOM | 429 | O | PHE | A | 93 | 95.906 | 41.237 | 4.294 | 1.00 | 57.05 |
| ATOM | 430 | N | LYS | A | 94 | 95.528 | 39.703 | 2.659 | 1.00 | 52.25 |
| ATOM | 431 | CA | LYS | A | 94 | 94.726 | 38.799 | 3.520 | 1.00 | 48.57 |
| ATOM | 432 | CB | LYS | A | 94 | 95.291 | 38.777 | 4.945 | 1.00 | 49.85 |
| ATOM | 433 | CG | LYS | A | 94 | 96.746 | 38.348 | 5.024 | 1.00 | 50.64 |
| ATOM | 434 | CD | LYS | A | 94 | 96.932 | 37.126 | 5.901 | 1.00 | 52.78 |
| ATOM | 435 | CE | LYS | A | 94 | 97.476 | 35.949 | 5.095 | 1.00 | 53.32 |
| ATOM | 436 | NZ | LYS | A | 94 | 98.246 | 35.007 | 5.950 | 1.00 | 55.33 |
| ATOM | 437 | C | LYS | A | 94 | 93.225 | 39.165 | 3.655 | 1.00 | 44.61 |
| ATOM | 438 | O | LYS | A | 94 | 92.892 | 40.177 | 4.271 | 1.00 | 45.08 |
| ATOM | 439 | N | ASN | A | 95 | 92.349 | 38.387 | 3.103 | 1.00 | 40.22 |
| ATOM | 440 | CA | ASN | A | 95 | 90.951 | 38.674 | 3.184 | 1.00 | 36.27 |
| ATOM | 441 | CB | ASN | A | 95 | 90.209 | 37.784 | 2.208 | 1.00 | 32.62 |
| ATOM | 442 | CG | ASN | A | 95 | 88.768 | 38.190 | 1.998 | 1.00 | 32.71 |
| ATOM | 443 | OD1 | ASN | A | 95 | 87.870 | 37.731 | 2.709 | 1.00 | 32.10 |
| ATOM | 444 | ND2 | ASN | A | 95 | 88.531 | 39.051 | 1.011 | 1.00 | 33.84 |
| ATOM | 445 | C | ASN | A | 95 | 90.380 | 38.559 | 4.600 | 1.00 | 33.71 |
| ATOM | 446 | O | ASN | A | 95 | 90.540 | 37.518 | 5.237 | 1.00 | 32.21 |
| ATOM | 447 | N | ARG | A | 96 | 89.731 | 39.618 | 5.080 | 1.00 | 31.07 |
| ATOM | 448 | CA | ARG | A | 96 | 89.146 | 39.634 | 6.408 | 1.00 | 28.30 |
| ATOM | 449 | CB | ARG | A | 96 | 88.612 | 41.034 | 6.729 | 1.00 | 28.81 |
| ATOM | 450 | CG | ARG | A | 96 | 88.371 | 41.285 | 8.216 | 1.00 | 27.99 |
| ATOM | 451 | CD | ARG | A | 96 | 89.015 | 42.582 | 8.681 | 1.00 | 28.70 |
| ATOM | 452 | NE | ARG | A | 96 | 88.085 | 43.704 | 8.620 | 1.00 | 28.92 |
| ATOM | 453 | CZ | ARG | A | 96 | 88.421 | 44.970 | 8.882 | 1.00 | 29.89 |
| ATOM | 454 | NH1 | ARG | A | 96 | 89.652 | 45.280 | 9.267 | 1.00 | 33.01 |
| ATOM | 455 | NH2 | ARG | A | 96 | 87.521 | 45.930 | 8.775 | 1.00 | 31.42 |
| ATOM | 456 | C | ARG | A | 96 | 88.031 | 38.615 | 6.512 | 1.00 | 28.03 |
| ATOM | 457 | O | ARG | A | 96 | 87.986 | 37.816 | 7.459 | 1.00 | 28.68 |
| ATOM | 458 | N | GLU | A | 97 | 87.124 | 38.644 | 5.534 | 1.00 | 25.77 |
| ATOM | 459 | CA | GLU | A | 97 | 85.994 | 37.730 | 5.507 | 1.00 | 22.33 |
| ATOM | 460 | CB | GLU | A | 97 | 85.089 | 38.035 | 4.313 | 1.00 | 24.09 |
| ATOM | 461 | CG | GLU | A | 97 | 83.803 | 37.214 | 4.227 | 1.00 | 21.86 |
| ATOM | 462 | CD | GLU | A | 97 | 82.859 | 37.724 | 3.140 | 1.00 | 21.24 |
| ATOM | 463 | OE1 | GLU | A | 97 | 81.665 | 37.342 | 3.150 | 1.00 | 19.24 |
| ATOM | 464 | OE2 | GLU | A | 97 | 83.314 | 38.514 | 2.284 | 1.00 | 21.60 |
| ATOM | 465 | C | GLU | A | 97 | 86.425 | 36.271 | 5.528 | 1.00 | 21.90 |
| ATOM | 466 | O | GLU | A | 97 | 85.815 | 35.451 | 6.219 | 1.00 | 24.32 |
| ATOM | 467 | N | LEU | A | 98 | 87.469 | 35.939 | 4.773 | 1.00 | 20.92 |
| ATOM | 468 | CA | LEU | A | 98 | 87.959 | 34.559 | 4.710 | 1.00 | 20.46 |
| ATOM | 469 | CB | LEU | A | 98 | 89.002 | 34.374 | 3.610 | 1.00 | 17.45 |
| ATOM | 470 | CG | LEU | A | 98 | 89.629 | 32.987 | 3.552 | 1.00 | 15.40 |
| ATOM | 471 | CD1 | LEU | A | 98 | 88.569 | 31.936 | 3.258 | 1.00 | 14.25 |
| ATOM | 472 | CD2 | LEU | A | 98 | 90.749 | 32.941 | 2.521 | 1.00 | 20.54 |
| ATOM | 473 | C | LEU | A | 98 | 88.532 | 34.070 | 6.043 | 1.00 | 20.25 |
| ATOM | 474 | O | LEU | A | 98 | 88.311 | 32.923 | 6.428 | 1.00 | 17.48 |
| ATOM | 475 | N | GLN | A | 99 | 89.280 | 34.937 | 6.719 | 1.00 | 21.83 |
| ATOM | 476 | CA | GLN | A | 99 | 89.890 | 34.584 | 7.996 | 1.00 | 25.86 |
| ATOM | 477 | CB | GLN | A | 99 | 90.957 | 35.602 | 8.415 | 1.00 | 28.40 |
| ATOM | 478 | CG | GLN | A | 99 | 92.051 | 35.830 | 7.369 | 1.00 | 34.01 |
| ATOM | 479 | CD | GLN | A | 99 | 92.960 | 34.619 | 7.138 | 1.00 | 38.37 |
| ATOM | 480 | OE1 | GLN | A | 99 | 94.019 | 34.487 | 7.763 | 1.00 | 40.68 |
| ATOM | 481 | NE2 | GLN | A | 99 | 92.562 | 33.738 | 6.219 | 1.00 | 39.28 |
| ATOM | 482 | C | GLN | A | 99 | 88.826 | 34.362 | 9.079 | 1.00 | 23.77 |
| ATOM | 483 | O | GLN | A | 99 | 88.952 | 33.458 | 9.892 | 1.00 | 26.29 |
| ATOM | 484 | N | ILE | A | 100 | 87.753 | 35.154 | 9.036 | 1.00 | 24.72 |
| ATOM | 485 | CA | ILE | A | 100 | 86.634 | 35.019 | 9.972 | 1.00 | 22.35 |
| ATOM | 486 | CB | ILE | A | 100 | 85.700 | 36.249 | 9.914 | 1.00 | 18.73 |
| ATOM | 487 | CG2 | ILE | A | 100 | 84.337 | 35.943 | 10.530 | 1.00 | 17.79 |
| ATOM | 488 | CG1 | ILE | A | 100 | 86.352 | 37.446 | 10.607 | 1.00 | 17.16 |
| ATOM | 489 | CD1 | ILE | A | 100 | 85.569 | 38.738 | 10.478 | 1.00 | 18.68 |
| ATOM | 490 | C | ILE | A | 100 | 85.820 | 33.758 | 9.676 | 1.00 | 25.52 |
| ATOM | 491 | O | ILE | A | 100 | 85.448 | 33.023 | 10.596 | 1.00 | 26.11 |
| ATOM | 492 | N | MET | A | 101 | 85.533 | 33.523 | 8.383 | 1.00 | 25.86 |
| ATOM | 493 | CA | MET | A | 101 | 84.746 | 32.368 | 7.930 | 1.00 | 25.27 |
| ATOM | 494 | CB | MET | A | 101 | 84.471 | 32.467 | 6.426 | 1.00 | 29.17 |
| ATOM | 495 | CG | MET | A | 101 | 83.524 | 33.583 | 6.040 | 1.00 | 27.98 |
| ATOM | 496 | SD | MET | A | 101 | 81.810 | 33.114 | 6.260 | 1.00 | 36.43 |
| ATOM | 497 | CE | MET | A | 101 | 81.013 | 34.091 | 4.977 | 1.00 | 35.05 |
| ATOM | 498 | C | MET | A | 101 | 85.404 | 31.024 | 8.243 | 1.00 | 25.14 |
| ATOM | 499 | O | MET | A | 101 | 84.729 | 30.062 | 8.618 | 1.00 | 25.95 |
| ATOM | 500 | N | ARG | A | 102 | 86.713 | 30.954 | 8.080 | 1.00 | 25.37 |
| ATOM | 501 | CA | ARG | A | 102 | 87.454 | 29.725 | 8.346 | 1.00 | 27.16 |
| ATOM | 502 | CB | ARG | A | 102 | 88.897 | 29.893 | 7.896 | 1.00 | 27.85 |
| ATOM | 503 | CG | ARG | A | 102 | 89.101 | 29.711 | 6.405 | 1.00 | 31.76 |
| ATOM | 504 | CD | ARG | A | 102 | 90.344 | 28.885 | 6.123 | 1.00 | 30.40 |
| ATOM | 505 | NE | ARG | A | 102 | 91.474 | 29.723 | 5.762 | 1.00 | 32.24 |
| ATOM | 506 | CZ | ARG | A | 102 | 92.682 | 29.251 | 5.484 | 1.00 | 31.50 |
| ATOM | 507 | NH1 | ARG | A | 102 | 92.924 | 27.946 | 5.561 | 1.00 | 33.59 |
| ATOM | 508 | NH2 | ARG | A | 102 | 93.653 | 30.085 | 5.129 | 1.00 | 33.23 |
| ATOM | 509 | C | ARG | A | 102 | 87.418 | 29.321 | 9.842 | 1.00 | 27.31 |
| ATOM | 510 | O | ARG | A | 102 | 87.675 | 28.167 | 10.172 | 1.00 | 27.37 |
| ATOM | 511 | N | LYS | A | 103 | 87.108 | 30.267 | 10.735 | 1.00 | 27.43 |
| ATOM | 512 | CA | LYS | A | 103 | 87.057 | 29.982 | 12.171 | 1.00 | 28.05 |
| ATOM | 513 | CB | LYS | A | 103 | 87.529 | 31.193 | 12.978 | 1.00 | 28.46 |
| ATOM | 514 | CG | LYS | A | 103 | 88.957 | 31.618 | 12.690 | 1.00 | 28.79 |
| ATOM | 515 | CD | LYS | A | 103 | 89.201 | 33.055 | 13.123 | 1.00 | 33.11 |
| ATOM | 516 | CE | LYS | A | 103 | 88.674 | 33.318 | 14.527 | 1.00 | 34.14 |
| ATOM | 517 | NZ | LYS | A | 103 | 89.068 | 34.665 | 15.016 | 1.00 | 35.09 |
| ATOM | 518 | C | LYS | A | 103 | 85.656 | 29.608 | 12.647 | 1.00 | 26.90 |
| ATOM | 519 | O | LYS | A | 103 | 85.500 | 29.105 | 13.761 | 1.00 | 27.60 |
| ATOM | 520 | N | LEU | A | 104 | 84.645 | 29.882 | 11.832 | 1.00 | 24.89 |
| ATOM | 521 | CA | LEU | A | 104 | 83.270 | 29.602 | 12.209 | 1.00 | 24.18 |
| ATOM | 522 | CB | LEU | A | 104 | 82.322 | 30.650 | 11.619 | 1.00 | 24.18 |
| ATOM | 523 | CG | LEU | A | 104 | 82.703 | 32.114 | 11.872 | 1.00 | 24.11 |
| ATOM | 524 | CD1 | LEU | A | 104 | 81.625 | 33.052 | 11.347 | 1.00 | 21.41 |
| ATOM | 525 | CD2 | LEU | A | 104 | 82.958 | 32.354 | 13.359 | 1.00 | 23.00 |
| ATOM | 526 | C | LEU | A | 104 | 82.804 | 28.201 | 11.838 | 1.00 | 26.40 |
| ATOM | 527 | O | LEU | A | 104 | 83.179 | 27.653 | 10.789 | 1.00 | 26.85 |
| ATOM | 528 | N | ASP | A | 105 | 81.947 | 27.648 | 12.705 | 1.00 | 23.78 |
| ATOM | 529 | CA | ASP | A | 105 | 81.353 | 26.331 | 12.533 | 1.00 | 22.44 |
| ATOM | 530 | CB | ASP | A | 105 | 82.361 | 25.215 | 12.822 | 1.00 | 23.57 |
| ATOM | 531 | CG | ASP | A | 105 | 81.780 | 23.820 | 12.627 | 1.00 | 23.62 |
| ATOM | 532 | OD1 | ASP | A | 105 | 80.616 | 23.699 | 12.193 | 1.00 | 28.28 |
| ATOM | 533 | OD2 | ASP | A | 105 | 82.487 | 22.847 | 12.911 | 1.00 | 28.66 |
| ATOM | 534 | C | ASP | A | 105 | 80.127 | 26.195 | 13.426 | 1.00 | 23.03 |
| ATOM | 535 | O | ASP | A | 105 | 80.180 | 25.590 | 14.497 | 1.00 | 24.10 |
| ATOM | 536 | N | HIS | A | 106 | 79.030 | 26.779 | 12.981 | 1.00 | 23.13 |
| ATOM | 537 | CA | HIS | A | 106 | 77.786 | 26.763 | 13.712 | 1.00 | 22.25 |
| ATOM | 538 | CB | HIS | A | 106 | 77.570 | 28.132 | 14.339 | 1.00 | 23.75 |
| ATOM | 539 | CG | HIS | A | 106 | 76.513 | 28.159 | 15.391 | 1.00 | 24.95 |
| ATOM | 540 | CD2 | HIS | A | 106 | 76.590 | 27.976 | 16.734 | 1.00 | 25.19 |
| ATOM | 541 | ND1 | HIS | A | 106 | 75.194 | 28.434 | 15.118 | 1.00 | 25.11 |
| ATOM | 542 | CE1 | HIS | A | 106 | 74.500 | 28.427 | 16.245 | 1.00 | 27.00 |
| ATOM | 543 | NE2 | HIS | A | 106 | 75.326 | 28.151 | 17.238 | 1.00 | 23.85 |
| ATOM | 544 | C | HIS | A | 106 | 76.613 | 26.415 | 12.801 | 1.00 | 24.48 |
| ATOM | 545 | O | HIS | A | 106 | 76.613 | 26.757 | 11.616 | 1.00 | 25.58 |
| ATOM | 546 | N | CYS | A | 107 | 75.617 | 25.729 | 13.367 | 1.00 | 24.64 |
| ATOM | 547 | CA | CYS | A | 107 | 74.425 | 25.300 | 12.644 | 1.00 | 25.69 |
| ATOM | 548 | CB | CYS | A | 107 | 73.612 | 24.293 | 13.478 | 1.00 | 28.55 |
| ATOM | 549 | SG | CYS | A | 107 | 72.821 | 24.992 | 14.957 | 1.00 | 36.02 |
| ATOM | 550 | C | CYS | A | 107 | 73.538 | 26.451 | 12.181 | 1.00 | 24.85 |
| ATOM | 551 | O | CYS | A | 107 | 72.648 | 26.245 | 11.355 | 1.00 | 24.42 |
| ATOM | 552 | N | ASN | A | 108 | 73.773 | 27.658 | 12.705 | 1.00 | 23.50 |
| ATOM | 553 | CA | ASN | A | 108 | 72.974 | 28.824 | 12.316 | 1.00 | 21.14 |
| ATOM | 554 | CB | ASN | A | 108 | 72.246 | 29.457 | 13.506 | 1.00 | 23.02 |
| ATOM | 555 | CG | ASN | A | 108 | 71.268 | 28.512 | 14.168 | 1.00 | 23.46 |
| ATOM | 556 | OD1 | ASN | A | 108 | 71.408 | 28.178 | 15.358 | 1.00 | 25.29 |
| ATOM | 557 | ND2 | ASN | A | 108 | 70.274 | 28.073 | 13.414 | 1.00 | 22.20 |
| ATOM | 558 | C | ASN | A | 108 | 73.807 | 29.861 | 11.571 | 1.00 | 18.73 |
| ATOM | 559 | O | ASN | A | 108 | 73.453 | 31.030 | 11.521 | 1.00 | 18.69 |
| ATOM | 560 | N | ILE | A | 109 | 74.898 | 29.410 | 10.971 | 1.00 | 20.13 |
| ATOM | 561 | CA | ILE | A | 109 | 75.785 | 30.267 | 10.184 | 1.00 | 20.40 |
| ATOM | 562 | CB | ILE | A | 109 | 77.101 | 30.556 | 10.918 | 1.00 | 20.32 |
| ATOM | 563 | CG2 | ILE | A | 109 | 78.010 | 31.422 | 10.052 | 1.00 | 18.74 |
| ATOM | 564 | CG1 | ILE | A | 109 | 76.842 | 31.217 | 12.291 | 1.00 | 19.54 |
| ATOM | 565 | CD1 | ILE | A | 109 | 78.073 | 31.857 | 12.894 | 1.00 | 15.97 |
| ATOM | 566 | C | ILE | A | 109 | 76.119 | 29.566 | 8.858 | 1.00 | 21.15 |
| ATOM | 567 | O | ILE | A | 109 | 76.427 | 28.366 | 8.853 | 1.00 | 20.11 |
| ATOM | 568 | N | VAL | A | 110 | 76.056 | 30.305 | 7.733 | 1.00 | 21.78 |
| ATOM | 569 | CA | VAL | A | 110 | 76.366 | 29.707 | 6.412 | 1.00 | 21.66 |
| ATOM | 570 | CB | VAL | A | 110 | 76.168 | 30.692 | 5.227 | 1.00 | 22.90 |
| ATOM | 571 | CG1 | VAL | A | 110 | 77.375 | 31.600 | 5.048 | 1.00 | 22.85 |
| ATOM | 572 | CG2 | VAL | A | 110 | 75.891 | 29.918 | 3.949 | 1.00 | 26.40 |
| ATOM | 573 | C | VAL | A | 110 | 77.773 | 29.149 | 6.401 | 1.00 | 19.25 |
| ATOM | 574 | O | VAL | A | 110 | 78.722 | 29.842 | 6.746 | 1.00 | 19.84 |
| ATOM | 575 | N | ARG | A | 111 | 77.894 | 27.886 | 6.036 | 1.00 | 19.86 |
| ATOM | 576 | CA | ARG | A | 111 | 79.184 | 27.207 | 6.015 | 1.00 | 21.87 |
| ATOM | 577 | CB | ARG | A | 111 | 78.955 | 25.702 | 6.024 | 1.00 | 24.97 |
| ATOM | 578 | CG | ARG | A | 111 | 80.229 | 24.888 | 6.100 | 1.00 | 29.38 |
| ATOM | 579 | CD | ARG | A | 111 | 79.924 | 23.401 | 6.091 | 1.00 | 34.23 |
| ATOM | 580 | NE | ARG | A | 111 | 81.103 | 22.604 | 5.751 | 1.00 | 37.78 |
| ATOM | 581 | CZ | ARG | A | 111 | 81.057 | 21.418 | 5.144 | 1.00 | 40.20 |
| ATOM | 582 | NH1 | ARG | A | 111 | 79.890 | 20.865 | 4.812 | 1.00 | 41.37 |
| ATOM | 583 | NH2 | ARG | A | 111 | 82.190 | 20.783 | 4.858 | 1.00 | 43.22 |
| ATOM | 584 | C | ARG | A | 111 | 80.053 | 27.564 | 4.797 | 1.00 | 23.84 |
| ATOM | 585 | O | ARG | A | 111 | 79.553 | 27.707 | 3.677 | 1.00 | 24.32 |
| ATOM | 586 | N | LEU | A | 112 | 81.366 | 27.646 | 5.044 | 1.00 | 22.96 |
| ATOM | 587 | CA | LEU | A | 112 | 82.370 | 27.918 | 4.031 | 1.00 | 22.62 |
| ATOM | 588 | CB | LEU | A | 112 | 83.455 | 28.848 | 4.578 | 1.00 | 20.55 |
| ATOM | 589 | CG | LEU | A | 112 | 84.707 | 29.053 | 3.717 | 1.00 | 22.70 |
| ATOM | 590 | CD1 | LEU | A | 112 | 84.370 | 29.778 | 2.415 | 1.00 | 23.13 |
| ATOM | 591 | CD2 | LEU | A | 112 | 85.769 | 29.814 | 4.495 | 1.00 | 21.66 |
| ATOM | 592 | C | LEU | A | 112 | 82.962 | 26.582 | 3.569 | 1.00 | 23.22 |
| ATOM | 593 | O | LEU | A | 112 | 83.854 | 26.014 | 4.207 | 1.00 | 22.92 |
| ATOM | 594 | N | ARG | A | 113 | 82.416 | 26.075 | 2.467 | 1.00 | 22.08 |
| ATOM | 595 | CA | ARG | A | 113 | 82.833 | 24.794 | 1.902 | 1.00 | 20.00 |
| ATOM | 596 | CB | ARG | A | 113 | 81.864 | 24.373 | 0.813 | 1.00 | 22.28 |
| ATOM | 597 | CG | ARG | A | 113 | 80.425 | 24.419 | 1.254 | 1.00 | 22.76 |
| ATOM | 598 | CD | ARG | A | 113 | 79.947 | 23.054 | 1.678 | 1.00 | 28.44 |
| ATOM | 599 | NE | ARG | A | 113 | 78.566 | 22.834 | 1.271 | 1.00 | 32.49 |
| ATOM | 600 | CZ | ARG | A | 113 | 78.119 | 21.724 | 0.683 | 1.00 | 33.10 |
| ATOM | 601 | NH1 | ARG | A | 113 | 78.949 | 20.723 | 0.386 | 1.00 | 33.75 |
| ATOM | 602 | NH2 | ARG | A | 113 | 76.831 | 21.621 | 0.392 | 1.00 | 35.17 |
| ATOM | 603 | C | ARG | A | 113 | 84.231 | 24.840 | 1.343 | 1.00 | 16.86 |
| ATOM | 604 | O | ARG | A | 113 | 85.025 | 23.938 | 1.578 | 1.00 | 15.68 |
| ATOM | 605 | N | TYR | A | 114 | 84.527 | 25.889 | 0.590 | 1.00 | 19.17 |
| ATOM | 606 | CA | TYR | A | 114 | 85.845 | 26.044 | −0.014 | 1.00 | 16.95 |
| ATOM | 607 | CB | TYR | A | 114 | 85.930 | 25.304 | −1.371 | 1.00 | 18.87 |
| ATOM | 608 | CG | TYR | A | 114 | 85.644 | 23.829 | −1.394 | 1.00 | 15.79 |
| ATOM | 609 | CD1 | TYR | A | 114 | 84.377 | 23.365 | −1.646 | 1.00 | 18.97 |
| ATOM | 610 | CE1 | TYR | A | 114 | 84.114 | 22.014 | −1.686 | 1.00 | 20.84 |
| ATOM | 611 | CD2 | TYR | A | 114 | 86.655 | 22.905 | −1.187 | 1.00 | 18.92 |
| ATOM | 612 | CE2 | TYR | A | 114 | 86.404 | 21.558 | −1.224 | 1.00 | 18.34 |
| ATOM | 613 | CZ | TYR | A | 114 | 85.133 | 21.119 | −1.474 | 1.00 | 21.38 |
| ATOM | 614 | OH | TYR | A | 114 | 84.871 | 19.769 | −1.505 | 1.00 | 26.51 |
| ATOM | 615 | C | TYR | A | 114 | 86.124 | 27.494 | −0.331 | 1.00 | 15.53 |
| ATOM | 616 | O | TYR | A | 114 | 85.227 | 28.321 | −0.402 | 1.00 | 17.41 |
| ATOM | 617 | N | PHE | A | 115 | 87.371 | 27.749 | −0.627 | 1.00 | 16.85 |
| ATOM | 618 | CA | PHE | A | 115 | 87.826 | 29.043 | −1.072 | 1.00 | 20.16 |
| ATOM | 619 | CB | PHE | A | 115 | 88.345 | 29.934 | 0.064 | 1.00 | 18.14 |
| ATOM | 620 | CG | PHE | A | 115 | 89.569 | 29.436 | 0.771 | 1.00 | 18.65 |
| ATOM | 621 | CD1 | PHE | A | 115 | 90.822 | 29.914 | 0.432 | 1.00 | 19.77 |
| ATOM | 622 | CD2 | PHE | A | 115 | 89.467 | 28.512 | 1.798 | 1.00 | 20.84 |
| ATOM | 623 | CE1 | PHE | A | 115 | 91.952 | 29.475 | 1.101 | 1.00 | 22.37 |
| ATOM | 624 | CE2 | PHE | A | 115 | 90.589 | 28.070 | 2.465 | 1.00 | 19.43 |
| ATOM | 625 | CZ | PHE | A | 115 | 91.832 | 28.550 | 2.119 | 1.00 | 20.49 |
| ATOM | 626 | C | PHE | A | 115 | 88.881 | 28.806 | −2.149 | 1.00 | 20.03 |
| ATOM | 627 | O | PHE | A | 115 | 89.624 | 27.837 | −2.078 | 1.00 | 18.98 |
| ATOM | 628 | N | PHE | A | 116 | 88.930 | 29.656 | −3.157 | 1.00 | 23.37 |
| ATOM | 629 | CA | PHE | A | 116 | 89.909 | 29.462 | −4.232 | 1.00 | 22.44 |
| ATOM | 630 | CB | PHE | A | 116 | 89.464 | 28.335 | −5.170 | 1.00 | 22.20 |
| ATOM | 631 | CG | PHE | A | 116 | 88.290 | 28.662 | −6.050 | 1.00 | 22.32 |
| ATOM | 632 | CD1 | PHE | A | 116 | 88.483 | 29.271 | −7.290 | 1.00 | 22.41 |
| ATOM | 633 | CD2 | PHE | A | 116 | 87.007 | 28.335 | −5.662 | 1.00 | 19.00 |
| ATOM | 634 | CE1 | PHE | A | 116 | 87.412 | 29.547 | −8.124 | 1.00 | 22.74 |
| ATOM | 635 | CE2 | PHE | A | 116 | 85.929 | 28.609 | −6.485 | 1.00 | 24.32 |
| ATOM | 636 | CZ | PHE | A | 116 | 86.131 | 29.216 | −7.726 | 1.00 | 21.99 |
| ATOM | 637 | C | PHE | A | 116 | 90.167 | 30.739 | −4.993 | 1.00 | 23.65 |
| ATOM | 638 | O | PHE | A | 116 | 89.309 | 31.614 | −5.067 | 1.00 | 23.70 |
| ATOM | 639 | N | TYR | A | 117 | 91.360 | 30.850 | −5.543 | 1.00 | 27.46 |
| ATOM | 640 | CA | TYR | A | 117 | 91.744 | 32.035 | −6.283 | 1.00 | 31.04 |
| ATOM | 641 | CB | TYR | A | 117 | 93.154 | 32.450 | −5.907 | 1.00 | 30.88 |
| ATOM | 642 | CG | TYR | A | 117 | 93.281 | 32.754 | −4.432 | 1.00 | 30.90 |
| ATOM | 643 | CD1 | TYR | A | 117 | 93.589 | 31.754 | −3.526 | 1.00 | 30.87 |
| ATOM | 644 | CE1 | TYR | A | 117 | 93.708 | 32.024 | −2.175 | 1.00 | 31.90 |
| ATOM | 645 | CD2 | TYR | A | 117 | 93.089 | 34.036 | −3.950 | 1.00 | 30.52 |
| ATOM | 646 | CE2 | TYR | A | 117 | 93.203 | 34.319 | −2.602 | 1.00 | 32.72 |
| ATOM | 647 | CZ | TYR | A | 117 | 93.515 | 33.307 | −1.718 | 1.00 | 32.15 |
| ATOM | 648 | OH | TYR | A | 117 | 93.647 | 33.582 | −0.381 | 1.00 | 31.58 |
| ATOM | 649 | C | TYR | A | 117 | 91.570 | 31.864 | −7.786 | 1.00 | 33.82 |
| ATOM | 650 | O | TYR | A | 117 | 91.556 | 30.744 | −8.299 | 1.00 | 34.45 |
| ATOM | 651 | N | SER | A | 118 | 91.406 | 32.993 | −8.473 | 1.00 | 37.08 |
| ATOM | 652 | CA | SER | A | 118 | 91.200 | 33.024 | −9.921 | 1.00 | 39.93 |
| ATOM | 653 | CB | SER | A | 118 | 89.703 | 32.964 | −10.247 | 1.00 | 38.74 |
| ATOM | 654 | OG | SER | A | 118 | 89.054 | 34.178 | −9.909 | 1.00 | 37.18 |
| ATOM | 655 | C | SER | A | 118 | 91.838 | 34.274 | −10.531 | 1.00 | 42.40 |
| ATOM | 656 | O | SER | A | 118 | 92.023 | 35.278 | −9.849 | 1.00 | 42.03 |
| ATOM | 657 | N | SER | A | 119 | 92.198 | 34.195 | −11.810 | 1.00 | 46.22 |
| ATOM | 658 | CA | SER | A | 119 | 92.852 | 35.316 | −12.492 | 1.00 | 50.06 |
| ATOM | 659 | CB | SER | A | 119 | 94.035 | 34.802 | −13.326 | 1.00 | 50.24 |
| ATOM | 660 | OG | SER | A | 119 | 94.416 | 33.499 | −12.924 | 1.00 | 50.86 |
| ATOM | 661 | C | SER | A | 119 | 91.910 | 36.117 | −13.385 | 1.00 | 52.37 |
| ATOM | 662 | O | SER | A | 119 | 91.903 | 37.347 | −13.341 | 1.00 | 52.91 |
| ATOM | 663 | N | GLY | A | 120 | 91.130 | 35.395 | −14.173 | 1.00 | 55.27 |
| ATOM | 664 | CA | GLY | A | 120 | 90.150 | 35.997 | −15.063 | 1.00 | 60.24 |
| ATOM | 665 | C | GLY | A | 120 | 90.723 | 36.319 | −16.427 | 1.00 | 63.01 |
| ATOM | 666 | O | GLY | A | 120 | 91.136 | 35.443 | −17.164 | 1.00 | 63.92 |
| ATOM | 667 | N | GLU | A | 121 | 90.729 | 37.605 | −16.750 | 1.00 | 64.87 |
| ATOM | 668 | CA | GLU | A | 121 | 91.181 | 38.035 | −18.059 | 1.00 | 66.91 |
| ATOM | 669 | CB | GLU | A | 121 | 90.002 | 38.239 | −19.003 | 1.00 | 67.13 |
| ATOM | 670 | CG | GLU | A | 121 | 89.872 | 37.163 | −20.053 | 1.00 | 67.59 |
| ATOM | 671 | CD | GLU | A | 121 | 88.551 | 36.456 | −19.879 | 1.00 | 67.97 |
| ATOM | 672 | OE1 | GLU | A | 121 | 88.199 | 36.107 | −18.728 | 1.00 | 68.36 |
| ATOM | 673 | OE2 | GLU | A | 121 | 87.845 | 36.238 | −20.894 | 1.00 | 67.22 |
| ATOM | 674 | C | GLU | A | 121 | 92.000 | 39.293 | −18.102 | 1.00 | 68.05 |
| ATOM | 675 | O | GLU | A | 121 | 91.527 | 40.416 | −18.130 | 1.00 | 68.41 |
| ATOM | 676 | N | LYS | A | 122 | 93.275 | 38.947 | −18.134 | 1.00 | 69.16 |
| ATOM | 677 | CA | LYS | A | 122 | 94.495 | 39.765 | −18.380 | 1.00 | 70.02 |
| ATOM | 678 | CB | LYS | A | 122 | 94.410 | 40.220 | −19.811 | 1.00 | 71.49 |
| ATOM | 679 | CG | LYS | A | 122 | 94.103 | 39.108 | −20.773 | 1.00 | 72.69 |
| ATOM | 680 | CD | LYS | A | 122 | 94.577 | 39.506 | −22.155 | 1.00 | 73.78 |
| ATOM | 681 | CE | LYS | A | 122 | 94.836 | 38.285 | −23.021 | 1.00 | 73.88 |
| ATOM | 682 | NZ | LYS | A | 122 | 94.892 | 38.640 | −24.471 | 1.00 | 74.66 |
| ATOM | 683 | C | LYS | A | 122 | 94.918 | 40.978 | −17.591 | 1.00 | 70.09 |
| ATOM | 684 | O | LYS | A | 122 | 94.764 | 42.098 | −18.090 | 1.00 | 71.62 |
| ATOM | 685 | N | LYS | A | 123 | 95.468 | 40.829 | −16.401 | 1.00 | 68.95 |
| ATOM | 686 | CA | LYS | A | 123 | 95.948 | 42.044 | −15.721 | 1.00 | 67.46 |
| ATOM | 687 | CB | LYS | A | 123 | 94.892 | 43.156 | −15.742 | 1.00 | 68.33 |
| ATOM | 688 | CG | LYS | A | 123 | 94.578 | 43.722 | −17.135 | 1.00 | 69.00 |
| ATOM | 689 | CD | LYS | A | 123 | 94.844 | 45.220 | −17.274 | 1.00 | 70.06 |
| ATOM | 690 | CE | LYS | A | 123 | 94.308 | 45.768 | −18.589 | 1.00 | 70.66 |
| ATOM | 691 | NZ | LYS | A | 123 | 94.406 | 47.255 | −18.659 | 1.00 | 71.09 |
| ATOM | 692 | C | LYS | A | 123 | 96.400 | 41.567 | −14.337 | 1.00 | 66.42 |
| ATOM | 693 | O | LYS | A | 123 | 96.248 | 40.376 | −14.078 | 1.00 | 66.52 |
| ATOM | 694 | N | ASP | A | 124 | 96.979 | 42.356 | −13.419 | 1.00 | 64.99 |
| ATOM | 695 | CA | ASP | A | 124 | 97.386 | 41.763 | −12.097 | 1.00 | 63.08 |
| ATOM | 696 | CB | ASP | A | 124 | 98.581 | 42.557 | −11.470 | 1.00 | 64.72 |
| ATOM | 697 | CG | ASP | A | 124 | 99.508 | 41.719 | −10.574 | 1.00 | 66.66 |
| ATOM | 698 | OD1 | ASP | A | 124 | 99.007 | 40.778 | −9.918 | 1.00 | 67.31 |
| ATOM | 699 | OD2 | ASP | A | 124 | 100.713 | 42.008 | −10.527 | 1.00 | 67.88 |
| ATOM | 700 | C | ASP | A | 124 | 96.195 | 41.642 | −11.147 | 1.00 | 60.58 |
| ATOM | 701 | O | ASP | A | 124 | 96.279 | 41.843 | −9.928 | 1.00 | 60.32 |
| ATOM | 702 | N | GLU | A | 125 | 95.069 | 41.253 | −11.806 | 1.00 | 57.75 |
| ATOM | 703 | CA | GLU | A | 125 | 93.726 | 41.062 | −11.257 | 1.00 | 54.51 |
| ATOM | 704 | CB | GLU | A | 125 | 92.680 | 41.433 | −12.338 | 1.00 | 54.64 |
| ATOM | 705 | CG | GLU | A | 125 | 92.520 | 40.487 | −13.535 | 1.00 | 55.43 |
| ATOM | 706 | CD | GLU | A | 125 | 91.258 | 40.811 | −14.361 | 1.00 | 57.22 |
| ATOM | 707 | OE1 | GLU | A | 125 | 90.365 | 41.499 | −13.828 | 1.00 | 57.06 |
| ATOM | 708 | OE2 | GLU | A | 125 | 91.178 | 40.368 | −15.531 | 1.00 | 56.74 |
| ATOM | 709 | C | GLU | A | 125 | 93.483 | 39.673 | −10.725 | 1.00 | 52.58 |
| ATOM | 710 | O | GLU | A | 125 | 93.181 | 38.746 | −11.478 | 1.00 | 51.91 |
| ATOM | 711 | N | VAL | A | 126 | 93.636 | 39.543 | −9.399 | 1.00 | 50.15 |
| ATOM | 712 | CA | VAL | A | 126 | 93.422 | 38.271 | −8.700 | 1.00 | 47.11 |
| ATOM | 713 | CB | VAL | A | 126 | 94.660 | 37.861 | −7.847 | 1.00 | 48.52 |
| ATOM | 714 | CG1 | VAL | A | 126 | 95.786 | 38.882 | −7.991 | 1.00 | 48.96 |
| ATOM | 715 | CG2 | VAL | A | 126 | 94.272 | 37.693 | −6.385 | 1.00 | 49.20 |
| ATOM | 716 | C | VAL | A | 126 | 92.139 | 38.282 | −7.867 | 1.00 | 44.12 |
| ATOM | 717 | O | VAL | A | 126 | 91.864 | 39.242 | −7.137 | 1.00 | 44.14 |
| ATOM | 718 | N | TYR | A | 127 | 91.348 | 37.221 | −8.001 | 1.00 | 39.73 |
| ATOM | 719 | CA | TYR | A | 127 | 90.085 | 37.115 | −7.283 | 1.00 | 36.96 |
| ATOM | 720 | CB | TYR | A | 127 | 88.927 | 36.904 | −8.257 | 1.00 | 37.95 |
| ATOM | 721 | CG | TYR | A | 127 | 88.736 | 38.015 | −9.267 | 1.00 | 40.29 |
| ATOM | 722 | CD1 | TYR | A | 127 | 89.358 | 37.961 | −10.510 | 1.00 | 40.59 |
| ATOM | 723 | CE1 | TYR | A | 127 | 89.181 | 38.966 | −11.439 | 1.00 | 42.77 |
| ATOM | 724 | CD2 | TYR | A | 127 | 87.928 | 39.107 | −8.982 | 1.00 | 39.85 |
| ATOM | 725 | CE2 | TYR | A | 127 | 87.745 | 40.119 | −9.904 | 1.00 | 41.70 |
| ATOM | 726 | CZ | TYR | A | 127 | 88.374 | 40.043 | −11.133 | 1.00 | 42.94 |
| ATOM | 727 | OH | TYR | A | 127 | 88.194 | 41.047 | −12.060 | 1.00 | 43.28 |
| ATOM | 728 | C | TYR | A | 127 | 90.062 | 35.982 | −6.260 | 1.00 | 34.06 |
| ATOM | 729 | O | TYR | A | 127 | 90.468 | 34.856 | −6.543 | 1.00 | 33.11 |
| ATOM | 730 | N | LEU | A | 128 | 89.502 | 36.285 | −5.089 | 1.00 | 31.02 |
| ATOM | 731 | CA | LEU | A | 128 | 89.322 | 35.294 | −4.033 | 1.00 | 25.83 |
| ATOM | 732 | CB | LEU | A | 128 | 89.494 | 35.929 | −2.643 | 1.00 | 23.26 |
| ATOM | 733 | CG | LEU | A | 128 | 88.909 | 35.128 | −1.464 | 1.00 | 22.22 |
| ATOM | 734 | CD1 | LEU | A | 128 | 89.607 | 33.777 | −1.324 | 1.00 | 16.00 |
| ATOM | 735 | CD2 | LEU | A | 128 | 89.012 | 35.928 | −0.174 | 1.00 | 22.36 |
| ATOM | 736 | C | LEU | A | 128 | 87.915 | 34.761 | −4.187 | 1.00 | 21.88 |
| ATOM | 737 | O | LEU | A | 128 | 86.983 | 35.529 | −4.414 | 1.00 | 23.70 |
| ATOM | 738 | N | ASN | A | 129 | 87.755 | 33.464 | −4.112 | 1.00 | 20.48 |
| ATOM | 739 | CA | ASN | A | 129 | 86.444 | 32.869 | −4.290 | 1.00 | 20.40 |
| ATOM | 740 | CB | ASN | A | 129 | 86.435 | 31.956 | −5.530 | 1.00 | 19.61 |
| ATOM | 741 | CG | ASN | A | 129 | 86.750 | 32.706 | −6.826 | 1.00 | 18.83 |
| ATOM | 742 | OD1 | ASN | A | 129 | 87.882 | 33.116 | −7.070 | 1.00 | 18.44 |
| ATOM | 743 | ND2 | ASN | A | 129 | 85.738 | 32.890 | −7.649 | 1.00 | 18.28 |
| ATOM | 744 | C | ASN | A | 129 | 86.016 | 32.089 | −3.050 | 1.00 | 20.47 |
| ATOM | 745 | O | ASN | A | 129 | 86.687 | 31.155 | −2.633 | 1.00 | 21.80 |
| ATOM | 746 | N | LEU | A | 130 | 84.889 | 32.481 | −2.487 | 1.00 | 20.29 |
| ATOM | 747 | CA | LEU | A | 130 | 84.336 | 31.832 | −1.310 | 1.00 | 20.70 |
| ATOM | 748 | CB | LEU | A | 130 | 83.798 | 32.884 | −0.336 | 1.00 | 19.16 |
| ATOM | 749 | CG | LEU | A | 130 | 84.842 | 33.845 | 0.246 | 1.00 | 20.53 |
| ATOM | 750 | CD1 | LEU | A | 130 | 84.192 | 34.844 | 1.188 | 1.00 | 19.74 |
| ATOM | 751 | CD2 | LEU | A | 130 | 85.944 | 33.083 | 0.952 | 1.00 | 19.17 |
| ATOM | 752 | C | LEU | A | 130 | 83.212 | 30.912 | −1.720 | 1.00 | 18.40 |
| ATOM | 753 | O | LEU | A | 130 | 82.218 | 31.364 | −2.267 | 1.00 | 20.36 |
| ATOM | 754 | N | VAL | A | 131 | 83.366 | 29.623 | −1.453 | 1.00 | 17.41 |
| ATOM | 755 | CA | VAL | A | 131 | 82.331 | 28.658 | −1.795 | 1.00 | 18.90 |
| ATOM | 756 | CB | VAL | A | 131 | 82.898 | 27.362 | −2.394 | 1.00 | 16.62 |
| ATOM | 757 | CG1 | VAL | A | 131 | 81.783 | 26.545 | −3.013 | 1.00 | 14.67 |
| ATOM | 758 | CG2 | VAL | A | 131 | 83.985 | 27.672 | −3.401 | 1.00 | 20.08 |
| ATOM | 759 | C | VAL | A | 131 | 81.495 | 28.332 | −0.565 | 1.00 | 20.30 |
| ATOM | 760 | O | VAL | A | 131 | 81.922 | 27.587 | 0.312 | 1.00 | 23.54 |
| ATOM | 761 | N | LEU | A | 132 | 80.328 | 28.932 | −0.514 | 1.00 | 22.83 |
| ATOM | 762 | CA | LEU | A | 132 | 79.389 | 28.805 | 0.581 | 1.00 | 24.00 |
| ATOM | 763 | CB | LEU | A | 132 | 78.898 | 30.211 | 0.947 | 1.00 | 21.47 |
| ATOM | 764 | CG | LEU | A | 132 | 79.998 | 31.233 | 1.282 | 1.00 | 24.67 |
| ATOM | 765 | CD1 | LEU | A | 132 | 79.629 | 32.628 | 0.781 | 1.00 | 22.71 |
| ATOM | 766 | CD2 | LEU | A | 132 | 80.285 | 31.245 | 2.784 | 1.00 | 20.84 |
| ATOM | 767 | C | LEU | A | 132 | 78.189 | 27.940 | 0.221 | 1.00 | 26.60 |
| ATOM | 768 | O | LEU | A | 132 | 77.920 | 27.667 | −0.956 | 1.00 | 26.94 |
| ATOM | 769 | N | ASP | A | 133 | 77.448 | 27.535 | 1.247 | 1.00 | 26.48 |
| ATOM | 770 | CA | ASP | A | 133 | 76.252 | 26.732 | 1.056 | 1.00 | 27.70 |
| ATOM | 771 | CB | ASP | A | 133 | 75.722 | 26.229 | 2.401 | 1.00 | 30.25 |
| ATOM | 772 | CG | ASP | A | 133 | 76.219 | 24.847 | 2.745 | 1.00 | 32.32 |
| ATOM | 773 | OD1 | ASP | A | 133 | 76.667 | 24.644 | 3.892 | 1.00 | 35.29 |
| ATOM | 774 | OD2 | ASP | A | 133 | 76.163 | 23.962 | 1.868 | 1.00 | 36.61 |
| ATOM | 775 | C | ASP | A | 133 | 75.197 | 27.592 | 0.393 | 1.00 | 26.66 |
| ATOM | 776 | O | ASP | A | 133 | 75.159 | 28.798 | 0.618 | 1.00 | 27.07 |
| ATOM | 777 | N | TYR | A | 134 | 74.350 | 26.980 | −0.431 | 1.00 | 26.84 |
| ATOM | 778 | CA | TYR | A | 134 | 73.311 | 27.734 | −1.127 | 1.00 | 25.55 |
| ATOM | 779 | CB | TYR | A | 134 | 73.220 | 27.337 | −2.614 | 1.00 | 24.53 |
| ATOM | 780 | CG | TYR | A | 134 | 72.150 | 28.097 | −3.358 | 1.00 | 22.49 |
| ATOM | 781 | CD1 | TYR | A | 134 | 72.355 | 29.410 | −3.749 | 1.00 | 24.18 |
| ATOM | 782 | CE1 | TYR | A | 134 | 71.368 | 30.126 | −4.404 | 1.00 | 26.88 |
| ATOM | 783 | CD2 | TYR | A | 134 | 70.931 | 27.515 | −3.640 | 1.00 | 23.64 |
| ATOM | 784 | CE2 | TYR | A | 134 | 69.940 | 28.218 | −4.301 | 1.00 | 27.34 |
| ATOM | 785 | CZ | TYR | A | 134 | 70.166 | 29.526 | −4.678 | 1.00 | 25.61 |
| ATOM | 786 | OH | TYR | A | 134 | 69.181 | 30.236 | −5.319 | 1.00 | 31.37 |
| ATOM | 787 | C | TYR | A | 134 | 71.950 | 27.607 | −0.453 | 1.00 | 23.77 |
| ATOM | 788 | O | TYR | A | 134 | 71.436 | 26.517 | −0.286 | 1.00 | 26.19 |
| ATOM | 789 | N | VAL | A | 135 | 71.378 | 28.747 | −0.092 | 1.00 | 24.90 |
| ATOM | 790 | CA | VAL | A | 135 | 70.068 | 28.829 | 0.555 | 1.00 | 25.98 |
| ATOM | 791 | CB | VAL | A | 135 | 70.231 | 29.060 | 2.090 | 1.00 | 26.32 |
| ATOM | 792 | CG1 | VAL | A | 135 | 68.921 | 28.837 | 2.825 | 1.00 | 23.53 |
| ATOM | 793 | CG2 | VAL | A | 135 | 71.303 | 28.131 | 2.646 | 1.00 | 23.34 |
| ATOM | 794 | C | VAL | A | 135 | 69.298 | 29.983 | −0.096 | 1.00 | 26.48 |
| ATOM | 795 | O | VAL | A | 135 | 69.670 | 31.140 | 0.057 | 1.00 | 28.19 |
| ATOM | 796 | N | PRO | A | 136 | 68.259 | 29.671 | −0.904 | 1.00 | 29.01 |
| ATOM | 797 | CD | PRO | A | 136 | 67.800 | 28.303 | −1.208 | 1.00 | 28.45 |
| ATOM | 798 | CA | PRO | A | 136 | 67.487 | 30.682 | −1.669 | 1.00 | 29.50 |
| ATOM | 799 | CB | PRO | A | 136 | 66.720 | 29.834 | −2.687 | 1.00 | 29.21 |
| ATOM | 800 | CG | PRO | A | 136 | 66.532 | 28.532 | −1.992 | 1.00 | 29.24 |
| ATOM | 801 | C | PRO | A | 136 | 66.516 | 31.544 | −0.869 | 1.00 | 27.47 |
| ATOM | 802 | O | PRO | A | 136 | 66.426 | 32.753 | −1.095 | 1.00 | 27.62 |
| ATOM | 803 | N | GLU | A | 137 | 65.768 | 30.946 | 0.037 | 1.00 | 26.71 |
| ATOM | 804 | CA | GLU | A | 137 | 64.814 | 31.738 | 0.804 | 1.00 | 26.93 |
| ATOM | 805 | CB | GLU | A | 137 | 63.741 | 30.858 | 1.440 | 1.00 | 28.68 |
| ATOM | 806 | CG | GLU | A | 137 | 62.415 | 30.879 | 0.699 | 1.00 | 33.07 |
| ATOM | 807 | CD | GLU | A | 137 | 61.715 | 32.215 | 0.777 | 1.00 | 34.24 |
| ATOM | 808 | OE1 | GLU | A | 137 | 62.373 | 33.253 | 0.555 | 1.00 | 36.39 |
| ATOM | 809 | OE2 | GLU | A | 137 | 60.504 | 32.225 | 1.056 | 1.00 | 36.16 |
| ATOM | 810 | C | GLU | A | 137 | 65.508 | 32.600 | 1.855 | 1.00 | 25.17 |
| ATOM | 811 | O | GLU | A | 137 | 66.524 | 32.214 | 2.423 | 1.00 | 24.62 |
| ATOM | 812 | N | THR | A | 138 | 64.941 | 33.767 | 2.108 | 1.00 | 25.35 |
| ATOM | 813 | CA | THR | A | 138 | 65.476 | 34.686 | 3.104 | 1.00 | 21.92 |
| ATOM | 814 | CB | THR | A | 138 | 66.266 | 35.838 | 2.485 | 1.00 | 21.16 |
| ATOM | 815 | OG1 | THR | A | 138 | 65.406 | 36.743 | 1.818 | 1.00 | 20.53 |
| ATOM | 816 | CG2 | THR | A | 138 | 67.387 | 35.427 | 1.560 | 1.00 | 23.02 |
| ATOM | 817 | C | THR | A | 138 | 64.341 | 35.254 | 3.922 | 1.00 | 19.62 |
| ATOM | 818 | O | THR | A | 138 | 63.196 | 35.241 | 3.489 | 1.00 | 19.25 |
| ATOM | 819 | N | VAL | A | 139 | 64.664 | 35.774 | 5.104 | 1.00 | 18.08 |
| ATOM | 820 | CA | VAL | A | 139 | 63.646 | 36.366 | 5.963 | 1.00 | 15.06 |
| ATOM | 821 | CB | VAL | A | 139 | 64.250 | 36.775 | 7.341 | 1.00 | 15.44 |
| ATOM | 822 | CG1 | VAL | A | 139 | 63.261 | 37.633 | 8.128 | 1.00 | 14.44 |
| ATOM | 823 | CG2 | VAL | A | 139 | 64.628 | 35.526 | 8.126 | 1.00 | 14.11 |
| ATOM | 824 | C | VAL | A | 139 | 63.030 | 37.580 | 5.273 | 1.00 | 13.45 |
| ATOM | 825 | O | VAL | A | 139 | 61.843 | 37.840 | 5.405 | 1.00 | 16.40 |
| ATOM | 826 | N | TYR | A | 140 | 63.850 | 38.304 | 4.519 | 1.00 | 15.51 |
| ATOM | 827 | CA | TYR | A | 140 | 63.393 | 39.478 | 3.777 | 1.00 | 18.60 |
| ATOM | 828 | CB | TYR | A | 140 | 64.572 | 40.136 | 3.040 | 1.00 | 19.07 |
| ATOM | 829 | CG | TYR | A | 140 | 64.188 | 41.209 | 2.042 | 1.00 | 22.33 |
| ATOM | 830 | CD1 | TYR | A | 140 | 64.026 | 42.528 | 2.441 | 1.00 | 21.83 |
| ATOM | 831 | CE1 | TYR | A | 140 | 63.653 | 43.515 | 1.548 | 1.00 | 25.48 |
| ATOM | 832 | CD2 | TYR | A | 140 | 63.968 | 40.894 | 0.696 | 1.00 | 24.28 |
| ATOM | 833 | CE2 | TYR | A | 140 | 63.597 | 41.874 | −0.215 | 1.00 | 25.55 |
| ATOM | 834 | CZ | TYR | A | 140 | 63.438 | 43.181 | 0.213 | 1.00 | 28.52 |
| ATOM | 835 | OH | TYR | A | 140 | 63.070 | 44.159 | −0.687 | 1.00 | 29.82 |
| ATOM | 836 | C | TYR | A | 140 | 62.269 | 39.096 | 2.802 | 1.00 | 19.79 |
| ATOM | 837 | O | TYR | A | 140 | 61.222 | 39.728 | 2.778 | 1.00 | 21.98 |
| ATOM | 838 | N | ARG | A | 141 | 62.494 | 38.046 | 2.015 | 1.00 | 24.14 |
| ATOM | 839 | CA | ARG | A | 141 | 61.498 | 37.576 | 1.040 | 1.00 | 26.04 |
| ATOM | 840 | CB | ARG | A | 141 | 62.068 | 36.432 | 0.193 | 1.00 | 29.54 |
| ATOM | 841 | CG | ARG | A | 141 | 62.762 | 36.907 | −1.071 | 1.00 | 35.28 |
| ATOM | 842 | CD | ARG | A | 141 | 63.184 | 35.740 | −1.960 | 1.00 | 40.58 |
| ATOM | 843 | NE | ARG | A | 141 | 63.685 | 36.203 | −3.259 | 1.00 | 46.87 |
| ATOM | 844 | CZ | ARG | A | 141 | 64.975 | 36.443 | −3.525 | 1.00 | 48.90 |
| ATOM | 845 | NH1 | ARG | A | 141 | 65.915 | 36.179 | −2.617 | 1.00 | 50.35 |
| ATOM | 846 | NH2 | ARG | A | 141 | 65.328 | 36.940 | −4.713 | 1.00 | 51.46 |
| ATOM | 847 | C | ARG | A | 141 | 60.220 | 37.117 | 1.719 | 1.00 | 25.46 |
| ATOM | 848 | O | ARG | A | 141 | 59.124 | 37.513 | 1.330 | 1.00 | 25.12 |
| ATOM | 849 | N | VAL | A | 142 | 60.362 | 36.278 | 2.740 | 1.00 | 27.17 |
| ATOM | 850 | CA | VAL | A | 142 | 59.209 | 35.771 | 3.473 | 1.00 | 24.40 |
| ATOM | 851 | CB | VAL | A | 142 | 59.649 | 34.807 | 4.594 | 1.00 | 24.71 |
| ATOM | 852 | CG1 | VAL | A | 142 | 58.477 | 34.432 | 5.487 | 1.00 | 24.45 |
| ATOM | 853 | CG2 | VAL | A | 142 | 60.295 | 33.562 | 3.995 | 1.00 | 25.79 |
| ATOM | 854 | C | VAL | A | 142 | 58.402 | 36.922 | 4.059 | 1.00 | 23.43 |
| ATOM | 855 | O | VAL | A | 142 | 57.177 | 36.899 | 4.030 | 1.00 | 26.36 |
| ATOM | 856 | N | ALA | A | 143 | 59.109 | 37.927 | 4.584 | 1.00 | 23.47 |
| ATOM | 857 | CA | ALA | A | 143 | 58.492 | 39.111 | 5.192 | 1.00 | 22.83 |
| ATOM | 858 | CB | ALA | A | 143 | 59.569 | 39.970 | 5.858 | 1.00 | 20.91 |
| ATOM | 859 | C | ALA | A | 143 | 57.693 | 39.918 | 4.176 | 1.00 | 20.62 |
| ATOM | 860 | O | ALA | A | 143 | 56.521 | 40.225 | 4.384 | 1.00 | 17.83 |
| ATOM | 861 | N | ARG | A | 144 | 58.325 | 40.237 | 3.052 | 1.00 | 24.31 |
| ATOM | 862 | CA | ARG | A | 144 | 57.650 | 40.983 | 1.977 | 1.00 | 23.67 |
| ATOM | 863 | CB | ARG | A | 144 | 58.635 | 41.269 | 0.844 | 1.00 | 26.35 |
| ATOM | 864 | CG | ARG | A | 144 | 58.023 | 42.018 | −0.327 | 1.00 | 29.59 |
| ATOM | 865 | CD | ARG | A | 144 | 59.045 | 42.234 | −1.425 | 1.00 | 29.52 |
| ATOM | 866 | NE | ARG | A | 144 | 59.470 | 40.973 | −2.016 | 1.00 | 32.38 |
| ATOM | 867 | CZ | ARG | A | 144 | 60.603 | 40.819 | −2.699 | 1.00 | 33.78 |
| ATOM | 868 | NH1 | ARG | A | 144 | 61.368 | 41.874 | −2.977 | 1.00 | 34.65 |
| ATOM | 869 | NH2 | ARG | A | 144 | 60.962 | 39.609 | −3.123 | 1.00 | 34.37 |
| ATOM | 870 | C | ARG | A | 144 | 56.453 | 40.190 | 1.469 | 1.00 | 22.19 |
| ATOM | 871 | O | ARG | A | 144 | 55.399 | 40.749 | 1.181 | 1.00 | 23.86 |
| ATOM | 872 | N | HIS | A | 145 | 56.611 | 38.876 | 1.413 | 1.00 | 23.70 |
| ATOM | 873 | CA | HIS | A | 145 | 55.545 | 37.970 | 0.988 | 1.00 | 26.97 |
| ATOM | 874 | CB | HIS | A | 145 | 56.062 | 36.525 | 1.009 | 1.00 | 30.84 |
| ATOM | 875 | CG | HIS | A | 145 | 55.056 | 35.464 | 0.707 | 1.00 | 36.75 |
| ATOM | 876 | CD2 | HIS | A | 145 | 54.720 | 34.344 | 1.401 | 1.00 | 38.92 |
| ATOM | 877 | ND1 | HIS | A | 145 | 54.269 | 35.458 | −0.435 | 1.00 | 39.38 |
| ATOM | 878 | CE1 | HIS | A | 145 | 53.495 | 34.383 | −0.429 | 1.00 | 40.00 |
| ATOM | 879 | NE2 | HIS | A | 145 | 53.751 | 33.691 | 0.677 | 1.00 | 41.00 |
| ATOM | 880 | C | HIS | A | 145 | 54.290 | 38.159 | 1.852 | 1.00 | 27.08 |
| ATOM | 881 | O | HIS | A | 145 | 53.188 | 38.312 | 1.320 | 1.00 | 26.89 |
| ATOM | 882 | N | TYR | A | 146 | 54.463 | 38.203 | 3.194 | 1.00 | 26.20 |
| ATOM | 883 | CA | TYR | A | 146 | 53.329 | 38.432 | 4.116 | 1.00 | 23.27 |
| ATOM | 884 | CB | TYR | A | 146 | 53.707 | 38.079 | 5.566 | 1.00 | 21.78 |
| ATOM | 885 | CG | TYR | A | 146 | 53.640 | 36.608 | 5.837 | 1.00 | 16.94 |
| ATOM | 886 | CD1 | TYR | A | 146 | 54.716 | 35.793 | 5.563 | 1.00 | 15.65 |
| ATOM | 887 | CE1 | TYR | A | 146 | 54.641 | 34.434 | 5.758 | 1.00 | 15.99 |
| ATOM | 888 | CD2 | TYR | A | 146 | 52.484 | 36.034 | 6.314 | 1.00 | 15.27 |
| ATOM | 889 | CE2 | TYR | A | 146 | 52.408 | 34.673 | 6.520 | 1.00 | 16.80 |
| ATOM | 890 | CZ | TYR | A | 146 | 53.485 | 33.884 | 6.238 | 1.00 | 14.45 |
| ATOM | 891 | OH | TYR | A | 146 | 53.403 | 32.526 | 6.431 | 1.00 | 18.28 |
| ATOM | 892 | C | TYR | A | 146 | 52.867 | 39.879 | 4.044 | 1.00 | 22.91 |
| ATOM | 893 | O | TYR | A | 146 | 51.681 | 40.168 | 4.096 | 1.00 | 22.73 |
| ATOM | 894 | N | SER | A | 147 | 53.827 | 40.780 | 3.913 | 1.00 | 25.91 |
| ATOM | 895 | CA | SER | A | 147 | 53.567 | 42.218 | 3.816 | 1.00 | 29.26 |
| ATOM | 896 | CB | SER | A | 147 | 54.904 | 42.962 | 3.732 | 1.00 | 29.44 |
| ATOM | 897 | OG | SER | A | 147 | 54.724 | 44.295 | 3.311 | 1.00 | 34.56 |
| ATOM | 898 | C | SER | A | 147 | 52.667 | 42.571 | 2.606 | 1.00 | 31.47 |
| ATOM | 899 | O | SER | A | 147 | 51.723 | 43.361 | 2.733 | 1.00 | 30.92 |
| ATOM | 900 | N | ARG | A | 148 | 52.959 | 41.979 | 1.436 | 1.00 | 32.82 |
| ATOM | 901 | CA | ARG | A | 148 | 52.172 | 42.240 | 0.221 | 1.00 | 34.38 |
| ATOM | 902 | CB | ARG | A | 148 | 52.801 | 41.544 | −0.979 | 1.00 | 35.40 |
| ATOM | 903 | CG | ARG | A | 148 | 53.649 | 42.476 | −1.818 | 1.00 | 35.95 |
| ATOM | 904 | CD | ARG | A | 148 | 54.822 | 41.747 | −2.445 | 1.00 | 37.90 |
| ATOM | 905 | NE | ARG | A | 148 | 55.806 | 42.672 | −2.977 | 1.00 | 38.82 |
| ATOM | 906 | CZ | ARG | A | 148 | 56.864 | 42.291 | −3.674 | 1.00 | 41.01 |
| ATOM | 907 | NH1 | ARG | A | 148 | 57.058 | 41.002 | −3.949 | 1.00 | 41.88 |
| ATOM | 908 | NH2 | ARG | A | 148 | 57.734 | 43.200 | −4.101 | 1.00 | 41.62 |
| ATOM | 909 | C | ARG | A | 148 | 50.714 | 41.802 | 0.385 | 1.00 | 34.50 |
| ATOM | 910 | O | ARG | A | 148 | 49.798 | 42.417 | −0.173 | 1.00 | 35.04 |
| ATOM | 911 | N | ALA | A | 149 | 50.492 | 40.766 | 1.179 | 1.00 | 33.08 |
| ATOM | 912 | CA | ALA | A | 149 | 49.136 | 40.306 | 1.429 | 1.00 | 32.56 |
| ATOM | 913 | CB | ALA | A | 149 | 49.116 | 38.800 | 1.624 | 1.00 | 32.41 |
| ATOM | 914 | C | ALA | A | 149 | 48.535 | 41.021 | 2.647 | 1.00 | 32.79 |
| ATOM | 915 | O | ALA | A | 149 | 47.471 | 40.639 | 3.124 | 1.00 | 33.04 |
| ATOM | 916 | N | LYS | A | 150 | 49.233 | 42.054 | 3.155 | 1.00 | 34.10 |
| ATOM | 917 | CA | LYS | A | 150 | 48.776 | 42.804 | 4.328 | 1.00 | 34.50 |
| ATOM | 918 | CB | LYS | A | 150 | 47.474 | 43.560 | 4.023 | 1.00 | 34.90 |
| ATOM | 919 | CG | LYS | A | 150 | 47.633 | 44.680 | 3.001 | 1.00 | 36.28 |
| ATOM | 920 | CD | LYS | A | 150 | 48.720 | 45.668 | 3.405 | 1.00 | 38.33 |
| ATOM | 921 | CE | LYS | A | 150 | 48.615 | 46.983 | 2.636 | 1.00 | 40.38 |
| ATOM | 922 | NZ | LYS | A | 150 | 49.673 | 47.109 | 1.583 | 1.00 | 41.77 |
| ATOM | 923 | C | LYS | A | 150 | 48.589 | 41.851 | 5.508 | 1.00 | 34.57 |
| ATOM | 924 | O | LYS | A | 150 | 47.651 | 41.981 | 6.293 | 1.00 | 34.54 |
| ATOM | 925 | N | GLN | A | 151 | 49.483 | 40.872 | 5.599 | 1.00 | 34.09 |
| ATOM | 926 | CA | GLN | A | 151 | 49.417 | 39.868 | 6.645 | 1.00 | 34.74 |
| ATOM | 927 | CB | GLN | A | 151 | 49.142 | 38.500 | 6.043 | 1.00 | 35.68 |
| ATOM | 928 | CG | GLN | A | 151 | 47.699 | 38.313 | 5.622 | 1.00 | 38.01 |
| ATOM | 929 | CD | GLN | A | 151 | 47.309 | 36.860 | 5.577 | 1.00 | 38.94 |
| ATOM | 930 | OE1 | GLN | A | 151 | 48.170 | 35.982 | 5.527 | 1.00 | 37.93 |
| ATOM | 931 | NE2 | GLN | A | 151 | 46.004 | 36.596 | 5.599 | 1.00 | 39.52 |
| ATOM | 932 | C | GLN | A | 151 | 50.669 | 39.818 | 7.501 | 1.00 | 34.35 |
| ATOM | 933 | O | GLN | A | 151 | 51.739 | 40.266 | 7.100 | 1.00 | 32.05 |
| ATOM | 934 | N | THR | A | 152 | 50.506 | 39.263 | 8.698 | 1.00 | 34.62 |
| ATOM | 935 | CA | THR | A | 152 | 51.588 | 39.132 | 9.661 | 1.00 | 34.27 |
| ATOM | 936 | CB | THR | A | 152 | 51.033 | 39.416 | 11.078 | 1.00 | 36.00 |
| ATOM | 937 | OG1 | THR | A | 152 | 50.127 | 40.515 | 11.083 | 1.00 | 37.75 |
| ATOM | 938 | CG2 | THR | A | 152 | 52.091 | 39.668 | 12.129 | 1.00 | 35.72 |
| ATOM | 939 | C | THR | A | 152 | 52.160 | 37.723 | 9.623 | 1.00 | 31.15 |
| ATOM | 940 | O | THR | A | 152 | 51.415 | 36.759 | 9.512 | 1.00 | 32.52 |
| ATOM | 941 | N | LEU | A | 153 | 53.471 | 37.604 | 9.766 | 1.00 | 30.45 |
| ATOM | 942 | CA | LEU | A | 153 | 54.108 | 36.292 | 9.808 | 1.00 | 30.39 |
| ATOM | 943 | CB | LEU | A | 153 | 55.623 | 36.452 | 9.713 | 1.00 | 32.03 |
| ATOM | 944 | CG | LEU | A | 153 | 56.444 | 35.160 | 9.724 | 1.00 | 32.38 |
| ATOM | 945 | CD1 | LEU | A | 153 | 56.138 | 34.318 | 8.494 | 1.00 | 33.99 |
| ATOM | 946 | CD2 | LEU | A | 153 | 57.931 | 35.469 | 9.808 | 1.00 | 33.12 |
| ATOM | 947 | C | LEU | A | 153 | 53.752 | 35.619 | 11.145 | 1.00 | 29.43 |
| ATOM | 948 | O | LEU | A | 153 | 53.857 | 36.243 | 12.195 | 1.00 | 28.63 |
| ATOM | 949 | N | PRO | A | 154 | 53.280 | 34.356 | 11.133 | 1.00 | 28.74 |
| ATOM | 950 | CD | PRO | A | 154 | 53.046 | 33.527 | 9.930 | 1.00 | 27.55 |
| ATOM | 951 | CA | PRO | A | 154 | 52.888 | 33.651 | 12.369 | 1.00 | 27.14 |
| ATOM | 952 | CB | PRO | A | 154 | 52.653 | 32.224 | 11.895 | 1.00 | 27.21 |
| ATOM | 953 | CG | PRO | A | 154 | 52.190 | 32.414 | 10.479 | 1.00 | 30.24 |
| ATOM | 954 | C | PRO | A | 154 | 53.966 | 33.717 | 13.452 | 1.00 | 23.50 |
| ATOM | 955 | O | PRO | A | 154 | 55.105 | 33.353 | 13.218 | 1.00 | 24.23 |
| ATOM | 956 | N | VAL | A | 155 | 53.579 | 34.204 | 14.634 | 1.00 | 22.72 |
| ATOM | 957 | CA | VAL | A | 155 | 54.476 | 34.360 | 15.800 | 1.00 | 20.53 |
| ATOM | 958 | CB | VAL | A | 155 | 53.685 | 34.678 | 17.102 | 1.00 | 23.34 |
| ATOM | 959 | CG1 | VAL | A | 155 | 54.507 | 34.387 | 18.361 | 1.00 | 24.21 |
| ATOM | 960 | CG2 | VAL | A | 155 | 53.226 | 36.124 | 17.092 | 1.00 | 20.95 |
| ATOM | 961 | C | VAL | A | 155 | 55.414 | 33.177 | 16.007 | 1.00 | 16.62 |
| ATOM | 962 | O | VAL | A | 155 | 56.576 | 33.366 | 16.328 | 1.00 | 20.84 |
| ATOM | 963 | N | ILE | A | 156 | 54.933 | 31.960 | 15.807 | 1.00 | 18.03 |
| ATOM | 964 | CA | ILE | A | 156 | 55.790 | 30.790 | 15.965 | 1.00 | 19.48 |
| ATOM | 965 | CB | ILE | A | 156 | 55.043 | 29.441 | 15.759 | 1.00 | 21.25 |
| ATOM | 966 | CG2 | ILE | A | 156 | 54.460 | 29.328 | 14.356 | 1.00 | 23.81 |
| ATOM | 967 | CG1 | ILE | A | 156 | 55.971 | 28.257 | 16.041 | 1.00 | 21.62 |
| ATOM | 968 | CD1 | ILE | A | 156 | 56.329 | 28.079 | 17.507 | 1.00 | 23.55 |
| ATOM | 969 | C | ILE | A | 156 | 57.032 | 30.878 | 15.064 | 1.00 | 21.28 |
| ATOM | 970 | O | ILE | A | 156 | 58.115 | 30.419 | 15.450 | 1.00 | 23.11 |
| ATOM | 971 | N | TYR | A | 157 | 56.875 | 31.498 | 13.869 | 1.00 | 21.30 |
| ATOM | 972 | CA | TYR | A | 157 | 57.993 | 31.669 | 12.920 | 1.00 | 20.67 |
| ATOM | 973 | CB | TYR | A | 157 | 57.466 | 31.982 | 11.503 | 1.00 | 22.40 |
| ATOM | 974 | CG | TYR | A | 157 | 56.837 | 30.800 | 10.804 | 1.00 | 20.61 |
| ATOM | 975 | CD1 | TYR | A | 157 | 55.507 | 30.824 | 10.439 | 1.00 | 23.85 |
| ATOM | 976 | CE1 | TYR | A | 157 | 54.922 | 29.748 | 9.808 | 1.00 | 26.60 |
| ATOM | 977 | CD2 | TYR | A | 157 | 57.578 | 29.665 | 10.519 | 1.00 | 22.96 |
| ATOM | 978 | CE2 | TYR | A | 157 | 57.003 | 28.581 | 9.889 | 1.00 | 26.14 |
| ATOM | 979 | CZ | TYR | A | 157 | 55.673 | 28.631 | 9.536 | 1.00 | 27.11 |
| ATOM | 980 | OH | TYR | A | 157 | 55.092 | 27.558 | 8.905 | 1.00 | 32.33 |
| ATOM | 981 | C | TYR | A | 157 | 58.907 | 32.791 | 13.388 | 1.00 | 17.50 |
| ATOM | 982 | O | TYR | A | 157 | 60.119 | 32.722 | 13.270 | 1.00 | 16.80 |
| ATOM | 983 | N | VAL | A | 158 | 58.317 | 33.824 | 13.939 | 1.00 | 17.44 |
| ATOM | 984 | CA | VAL | A | 158 | 59.108 | 34.928 | 14.442 | 1.00 | 18.24 |
| ATOM | 985 | CB | VAL | A | 158 | 58.214 | 36.067 | 14.939 | 1.00 | 20.43 |
| ATOM | 986 | CG1 | VAL | A | 158 | 59.055 | 37.177 | 15.559 | 1.00 | 19.36 |
| ATOM | 987 | CG2 | VAL | A | 158 | 57.359 | 36.605 | 13.801 | 1.00 | 18.19 |
| ATOM | 988 | C | VAL | A | 158 | 60.014 | 34.432 | 15.574 | 1.00 | 17.07 |
| ATOM | 989 | O | VAL | A | 158 | 61.174 | 34.805 | 15.658 | 1.00 | 18.46 |
| ATOM | 990 | N | LYS | A | 159 | 59.480 | 33.560 | 16.428 | 1.00 | 18.82 |
| ATOM | 991 | CA | LYS | A | 159 | 60.264 | 33.002 | 17.545 | 1.00 | 19.42 |
| ATOM | 992 | CB | LYS | A | 159 | 59.362 | 32.146 | 18.441 | 1.00 | 16.16 |
| ATOM | 993 | CG | LYS | A | 159 | 58.585 | 32.971 | 19.446 | 1.00 | 18.16 |
| ATOM | 994 | CD | LYS | A | 159 | 57.569 | 32.135 | 20.217 | 1.00 | 19.52 |
| ATOM | 995 | CE | LYS | A | 159 | 56.559 | 33.019 | 20.938 | 1.00 | 20.28 |
| ATOM | 996 | NZ | LYS | A | 159 | 55.522 | 32.220 | 21.646 | 1.00 | 22.23 |
| ATOM | 997 | C | LYS | A | 159 | 61.434 | 32.179 | 17.050 | 1.00 | 18.62 |
| ATOM | 998 | O | LYS | A | 159 | 62.589 | 32.436 | 17.411 | 1.00 | 18.36 |
| ATOM | 999 | N | LEU | A | 160 | 61.121 | 31.178 | 16.225 | 1.00 | 20.14 |
| ATOM | 1000 | CA | LEU | A | 160 | 62.120 | 30.274 | 15.661 | 1.00 | 19.50 |
| ATOM | 1001 | CB | LEU | A | 160 | 61.466 | 29.267 | 14.706 | 1.00 | 19.84 |
| ATOM | 1002 | CG | LEU | A | 160 | 60.808 | 28.051 | 15.365 | 1.00 | 22.33 |
| ATOM | 1003 | CD1 | LEU | A | 160 | 59.638 | 27.550 | 14.528 | 1.00 | 22.99 |
| ATOM | 1004 | CD2 | LEU | A | 160 | 61.828 | 26.945 | 15.596 | 1.00 | 22.73 |
| ATOM | 1005 | C | LEU | A | 160 | 63.224 | 31.012 | 14.948 | 1.00 | 18.91 |
| ATOM | 1006 | O | LEU | A | 160 | 64.397 | 30.708 | 15.142 | 1.00 | 19.83 |
| ATOM | 1007 | N | TYR | A | 161 | 62.860 | 31.967 | 14.113 | 1.00 | 17.95 |
| ATOM | 1008 | CA | TYR | A | 161 | 63.865 | 32.719 | 13.366 | 1.00 | 20.81 |
| ATOM | 1009 | CB | TYR | A | 161 | 63.218 | 33.585 | 12.257 | 1.00 | 21.26 |
| ATOM | 1010 | CG | TYR | A | 161 | 62.470 | 32.792 | 11.198 | 1.00 | 23.48 |
| ATOM | 1011 | CD1 | TYR | A | 161 | 62.769 | 31.463 | 10.954 | 1.00 | 23.93 |
| ATOM | 1012 | CE1 | TYR | A | 161 | 62.064 | 30.738 | 10.009 | 1.00 | 29.23 |
| ATOM | 1013 | CD2 | TYR | A | 161 | 61.450 | 33.376 | 10.466 | 1.00 | 24.92 |
| ATOM | 1014 | CE2 | TYR | A | 161 | 60.744 | 32.665 | 9.516 | 1.00 | 27.72 |
| ATOM | 1015 | CZ | TYR | A | 161 | 61.051 | 31.348 | 9.291 | 1.00 | 28.92 |
| ATOM | 1016 | OH | TYR | A | 161 | 60.343 | 30.631 | 8.348 | 1.00 | 32.19 |
| ATOM | 1017 | C | TYR | A | 161 | 64.725 | 33.592 | 14.267 | 1.00 | 19.07 |
| ATOM | 1018 | O | TYR | A | 161 | 65.943 | 33.579 | 14.159 | 1.00 | 20.35 |
| ATOM | 1019 | N | MET | A | 162 | 64.078 | 34.382 | 15.130 | 1.00 | 19.27 |
| ATOM | 1020 | CA | MET | A | 162 | 64.808 | 35.296 | 16.026 | 1.00 | 17.97 |
| ATOM | 1021 | CB | MET | A | 162 | 63.843 | 36.243 | 16.743 | 1.00 | 18.49 |
| ATOM | 1022 | CG | MET | A | 162 | 63.234 | 37.287 | 15.810 | 1.00 | 20.41 |
| ATOM | 1023 | SD | MET | A | 162 | 64.483 | 38.213 | 14.898 | 1.00 | 22.51 |
| ATOM | 1024 | CE | MET | A | 162 | 65.609 | 38.696 | 16.222 | 1.00 | 21.98 |
| ATOM | 1025 | C | MET | A | 162 | 65.717 | 34.556 | 16.997 | 1.00 | 14.30 |
| ATOM | 1026 | O | MET | A | 162 | 66.844 | 34.977 | 17.247 | 1.00 | 18.15 |
| ATOM | 1027 | N | TYR | A | 163 | 65.250 | 33.433 | 17.508 | 1.00 | 15.87 |
| ATOM | 1028 | CA | TYR | A | 163 | 66.055 | 32.616 | 18.431 | 1.00 | 17.26 |
| ATOM | 1029 | CB | TYR | A | 163 | 65.225 | 31.385 | 18.868 | 1.00 | 16.49 |
| ATOM | 1030 | CG | TYR | A | 163 | 65.952 | 30.442 | 19.801 | 1.00 | 17.42 |
| ATOM | 1031 | CD1 | TYR | A | 163 | 65.926 | 30.620 | 21.194 | 1.00 | 18.67 |
| ATOM | 1032 | CE1 | TYR | A | 163 | 66.623 | 29.762 | 22.037 | 1.00 | 16.89 |
| ATOM | 1033 | CD2 | TYR | A | 163 | 66.684 | 29.381 | 19.291 | 1.00 | 16.80 |
| ATOM | 1034 | CE2 | TYR | A | 163 | 67.371 | 28.525 | 20.111 | 1.00 | 18.00 |
| ATOM | 1035 | CZ | TYR | A | 163 | 67.341 | 28.722 | 21.492 | 1.00 | 20.67 |
| ATOM | 1036 | OH | TYR | A | 163 | 68.062 | 27.886 | 22.302 | 1.00 | 20.49 |
| ATOM | 1037 | C | TYR | A | 163 | 67.367 | 32.182 | 17.770 | 1.00 | 18.07 |
| ATOM | 1038 | O | TYR | A | 163 | 68.480 | 32.385 | 18.309 | 1.00 | 20.01 |
| ATOM | 1039 | N | GLN | A | 164 | 67.244 | 31.585 | 16.579 | 1.00 | 17.19 |
| ATOM | 1040 | CA | GLN | A | 164 | 68.410 | 31.118 | 15.826 | 1.00 | 12.78 |
| ATOM | 1041 | CB | GLN | A | 164 | 67.966 | 30.351 | 14.569 | 1.00 | 14.61 |
| ATOM | 1042 | CG | GLN | A | 164 | 67.389 | 28.990 | 14.899 | 1.00 | 14.23 |
| ATOM | 1043 | CD | GLN | A | 164 | 66.632 | 28.353 | 13.751 | 1.00 | 16.46 |
| ATOM | 1044 | OE1 | GLN | A | 164 | 65.450 | 28.584 | 13.578 | 1.00 | 17.33 |
| ATOM | 1045 | NE2 | GLN | A | 164 | 67.312 | 27.522 | 12.981 | 1.00 | 12.47 |
| ATOM | 1046 | C | GLN | A | 164 | 69.396 | 32.233 | 15.520 | 1.00 | 8.49 |
| ATOM | 1047 | O | GLN | A | 164 | 70.590 | 32.015 | 15.536 | 1.00 | 9.26 |
| ATOM | 1048 | N | LEU | A | 165 | 68.883 | 33.437 | 15.282 | 1.00 | 11.16 |
| ATOM | 1049 | CA | LEU | A | 165 | 69.712 | 34.619 | 15.011 | 1.00 | 11.15 |
| ATOM | 1050 | CB | LEU | A | 165 | 68.797 | 35.799 | 14.635 | 1.00 | 9.98 |
| ATOM | 1051 | CG | LEU | A | 165 | 69.420 | 36.939 | 13.825 | 1.00 | 13.67 |
| ATOM | 1052 | CD1 | LEU | A | 165 | 68.899 | 38.285 | 14.272 | 1.00 | 13.53 |
| ATOM | 1053 | CD2 | LEU | A | 165 | 70.930 | 36.916 | 13.817 | 1.00 | 14.22 |
| ATOM | 1054 | C | LEU | A | 165 | 70.499 | 35.006 | 16.266 | 1.00 | 10.07 |
| ATOM | 1055 | O | LEU | A | 165 | 71.696 | 35.258 | 16.226 | 1.00 | 11.99 |
| ATOM | 1056 | N | PHE | A | 166 | 69.806 | 35.058 | 17.391 | 1.00 | 12.97 |
| ATOM | 1057 | CA | PHE | A | 166 | 70.474 | 35.408 | 18.652 | 1.00 | 13.16 |
| ATOM | 1058 | CB | PHE | A | 166 | 69.441 | 35.524 | 19.778 | 1.00 | 9.30 |
| ATOM | 1059 | CG | PHE | A | 166 | 68.706 | 36.825 | 19.704 | 1.00 | 5.00 |
| ATOM | 1060 | CD1 | PHE | A | 166 | 67.345 | 36.861 | 19.600 | 1.00 | 5.00 |
| ATOM | 1061 | CD2 | PHE | A | 166 | 69.396 | 38.007 | 19.743 | 1.00 | 5.00 |
| ATOM | 1062 | CE1 | PHE | A | 166 | 66.673 | 38.054 | 19.544 | 1.00 | 5.00 |
| ATOM | 1063 | CE2 | PHE | A | 166 | 68.733 | 39.208 | 19.682 | 1.00 | 7.78 |
| ATOM | 1064 | CZ | PHE | A | 166 | 67.370 | 39.228 | 19.586 | 1.00 | 6.07 |
| ATOM | 1065 | C | PHE | A | 166 | 71.557 | 34.396 | 18.943 | 1.00 | 9.24 |
| ATOM | 1066 | O | PHE | A | 166 | 72.679 | 34.743 | 19.273 | 1.00 | 14.23 |
| ATOM | 1067 | N | ARG | A | 167 | 71.238 | 33.147 | 18.736 | 1.00 | 11.80 |
| ATOM | 1068 | CA | ARG | A | 167 | 72.210 | 32.087 | 18.915 | 1.00 | 14.27 |
| ATOM | 1069 | CB | ARG | A | 167 | 71.542 | 30.754 | 18.618 | 1.00 | 17.59 |
| ATOM | 1070 | CG | ARG | A | 167 | 71.692 | 29.753 | 19.734 | 1.00 | 20.33 |
| ATOM | 1071 | CD | ARG | A | 167 | 72.416 | 28.515 | 19.268 | 1.00 | 19.92 |
| ATOM | 1072 | NE | ARG | A | 167 | 71.499 | 27.404 | 19.111 | 1.00 | 20.11 |
| ATOM | 1073 | CZ | ARG | A | 167 | 71.873 | 26.132 | 19.040 | 1.00 | 19.61 |
| ATOM | 1074 | NH1 | ARG | A | 167 | 73.150 | 25.796 | 19.095 | 1.00 | 20.92 |
| ATOM | 1075 | NH2 | ARG | A | 167 | 70.950 | 25.191 | 18.916 | 1.00 | 23.68 |
| ATOM | 1076 | C | ARG | A | 167 | 73.439 | 32.284 | 18.020 | 1.00 | 18.46 |
| ATOM | 1077 | O | ARG | A | 167 | 74.591 | 32.262 | 18.498 | 1.00 | 19.88 |
| ATOM | 1078 | N | SER | A | 168 | 73.224 | 32.492 | 16.707 | 1.00 | 18.03 |
| ATOM | 1079 | CA | SER | A | 168 | 74.380 | 32.704 | 15.820 | 1.00 | 16.37 |
| ATOM | 1080 | CB | SER | A | 168 | 73.950 | 32.854 | 14.337 | 1.00 | 13.75 |
| ATOM | 1081 | OG | SER | A | 168 | 73.376 | 34.123 | 14.092 | 1.00 | 10.05 |
| ATOM | 1082 | C | SER | A | 168 | 75.171 | 33.928 | 16.291 | 1.00 | 14.44 |
| ATOM | 1083 | O | SER | A | 168 | 76.397 | 33.940 | 16.249 | 1.00 | 15.57 |
| ATOM | 1084 | N | LEU | A | 169 | 74.460 | 34.952 | 16.763 | 1.00 | 13.04 |
| ATOM | 1085 | CA | LEU | A | 169 | 75.127 | 36.170 | 17.264 | 1.00 | 15.39 |
| ATOM | 1086 | CB | LEU | A | 169 | 74.098 | 37.253 | 17.578 | 1.00 | 15.44 |
| ATOM | 1087 | CG | LEU | A | 169 | 73.461 | 37.955 | 16.369 | 1.00 | 17.31 |
| ATOM | 1088 | CD1 | LEU | A | 169 | 72.451 | 38.999 | 16.826 | 1.00 | 12.89 |
| ATOM | 1089 | CD2 | LEU | A | 169 | 74.535 | 38.608 | 15.514 | 1.00 | 12.28 |
| ATOM | 1090 | C | LEU | A | 169 | 75.979 | 35.855 | 18.511 | 1.00 | 14.46 |
| ATOM | 1091 | O | LEU | A | 169 | 77.130 | 36.263 | 18.611 | 1.00 | 14.94 |
| ATOM | 1092 | N | ALA | A | 170 | 75.413 | 35.083 | 19.438 | 1.00 | 17.16 |
| ATOM | 1093 | CA | ALA | A | 170 | 76.135 | 34.678 | 20.653 | 1.00 | 18.82 |
| ATOM | 1094 | CB | ALA | A | 170 | 75.251 | 33.764 | 21.513 | 1.00 | 18.42 |
| ATOM | 1095 | C | ALA | A | 170 | 77.425 | 33.964 | 20.279 | 1.00 | 18.95 |
| ATOM | 1096 | O | ALA | A | 170 | 78.518 | 34.272 | 20.800 | 1.00 | 19.35 |
| ATOM | 1097 | N | TYR | A | 171 | 77.303 | 33.011 | 19.358 | 1.00 | 19.08 |
| ATOM | 1098 | CA | TYR | A | 171 | 78.455 | 32.251 | 18.897 | 1.00 | 16.90 |
| ATOM | 1099 | CB | TYR | A | 171 | 78.033 | 31.178 | 17.884 | 1.00 | 17.64 |
| ATOM | 1100 | CG | TYR | A | 171 | 79.187 | 30.407 | 17.276 | 1.00 | 17.25 |
| ATOM | 1101 | CD1 | TYR | A | 171 | 79.791 | 29.354 | 17.957 | 1.00 | 15.43 |
| ATOM | 1102 | CE1 | TYR | A | 171 | 80.836 | 28.648 | 17.394 | 1.00 | 17.19 |
| ATOM | 1103 | CD2 | TYR | A | 171 | 79.667 | 30.735 | 16.013 | 1.00 | 15.34 |
| ATOM | 1104 | CE2 | TYR | A | 171 | 80.717 | 30.035 | 15.439 | 1.00 | 15.84 |
| ATOM | 1105 | CZ | TYR | A | 171 | 81.294 | 28.998 | 16.125 | 1.00 | 18.87 |
| ATOM | 1106 | OH | TYR | A | 171 | 82.328 | 28.299 | 15.551 | 1.00 | 20.53 |
| ATOM | 1107 | C | TYR | A | 171 | 79.551 | 33.149 | 18.345 | 1.00 | 16.46 |
| ATOM | 1108 | O | TYR | A | 171 | 80.717 | 33.012 | 18.719 | 1.00 | 18.03 |
| ATOM | 1109 | N | ILE | A | 172 | 79.203 | 34.073 | 17.449 | 1.00 | 16.80 |
| ATOM | 1110 | CA | ILE | A | 172 | 80.235 | 34.947 | 16.878 | 1.00 | 16.95 |
| ATOM | 1111 | CB | ILE | A | 172 | 79.798 | 35.670 | 15.572 | 1.00 | 15.99 |
| ATOM | 1112 | CG2 | ILE | A | 172 | 79.655 | 34.672 | 14.435 | 1.00 | 18.73 |
| ATOM | 1113 | CG1 | ILE | A | 172 | 78.488 | 36.437 | 15.769 | 1.00 | 16.30 |
| ATOM | 1114 | CD1 | ILE | A | 172 | 78.254 | 37.509 | 14.728 | 1.00 | 18.08 |
| ATOM | 1115 | C | ILE | A | 172 | 80.744 | 35.949 | 17.892 | 1.00 | 15.06 |
| ATOM | 1116 | O | ILE | A | 172 | 81.937 | 36.187 | 17.987 | 1.00 | 14.81 |
| ATOM | 1117 | N | HIS | A | 173 | 79.834 | 36.527 | 18.662 | 1.00 | 16.56 |
| ATOM | 1118 | CA | HIS | A | 173 | 80.230 | 37.494 | 19.692 | 1.00 | 17.03 |
| ATOM | 1119 | CB | HIS | A | 173 | 78.991 | 38.097 | 20.377 | 1.00 | 16.14 |
| ATOM | 1120 | CG | HIS | A | 173 | 78.208 | 39.002 | 19.467 | 1.00 | 14.02 |
| ATOM | 1121 | CD2 | HIS | A | 173 | 78.483 | 39.454 | 18.197 | 1.00 | 13.45 |
| ATOM | 1122 | ND1 | HIS | A | 173 | 77.015 | 39.594 | 19.825 | 1.00 | 12.19 |
| ATOM | 1123 | CE1 | HIS | A | 173 | 76.587 | 40.372 | 18.826 | 1.00 | 14.81 |
| ATOM | 1124 | NE2 | HIS | A | 173 | 77.466 | 40.300 | 17.831 | 1.00 | 10.44 |
| ATOM | 1125 | C | HIS | A | 173 | 81.225 | 36.849 | 20.687 | 1.00 | 17.24 |
| ATOM | 1126 | O | HIS | A | 173 | 82.097 | 37.540 | 21.205 | 1.00 | 20.12 |
| ATOM | 1127 | N | SER | A | 174 | 81.130 | 35.508 | 20.877 | 1.00 | 17.18 |
| ATOM | 1128 | CA | SER | A | 174 | 82.043 | 34.715 | 21.747 | 1.00 | 18.95 |
| ATOM | 1129 | CB | SER | A | 174 | 81.725 | 33.214 | 21.672 | 1.00 | 18.66 |
| ATOM | 1130 | OG | SER | A | 174 | 80.376 | 32.963 | 21.936 | 1.00 | 29.03 |
| ATOM | 1131 | C | SER | A | 174 | 83.499 | 34.857 | 21.344 | 1.00 | 20.81 |
| ATOM | 1132 | O | SER | A | 174 | 84.400 | 34.758 | 22.196 | 1.00 | 18.20 |
| ATOM | 1133 | N | PHE | A | 175 | 83.732 | 35.042 | 20.035 | 1.00 | 18.33 |
| ATOM | 1134 | CA | PHE | A | 175 | 85.079 | 35.164 | 19.488 | 1.00 | 18.92 |
| ATOM | 1135 | CB | PHE | A | 175 | 85.156 | 34.469 | 18.111 | 1.00 | 20.09 |
| ATOM | 1136 | CG | PHE | A | 175 | 85.081 | 32.969 | 18.177 | 1.00 | 18.05 |
| ATOM | 1137 | CD1 | PHE | A | 175 | 86.215 | 32.221 | 18.380 | 1.00 | 18.98 |
| ATOM | 1138 | CD2 | PHE | A | 175 | 83.875 | 32.317 | 18.051 | 1.00 | 19.83 |
| ATOM | 1139 | CE1 | PHE | A | 175 | 86.151 | 30.849 | 18.455 | 1.00 | 19.35 |
| ATOM | 1140 | CE2 | PHE | A | 175 | 83.804 | 30.945 | 18.124 | 1.00 | 19.15 |
| ATOM | 1141 | CZ | PHE | A | 175 | 84.943 | 30.211 | 18.327 | 1.00 | 17.42 |
| ATOM | 1142 | C | PHE | A | 175 | 85.484 | 36.615 | 19.330 | 1.00 | 18.22 |
| ATOM | 1143 | O | PHE | A | 175 | 86.571 | 36.903 | 18.831 | 1.00 | 21.16 |
| ATOM | 1144 | N | GLY | A | 176 | 84.599 | 37.528 | 19.727 | 1.00 | 18.25 |
| ATOM | 1145 | CA | GLY | A | 176 | 84.878 | 38.955 | 19.591 | 1.00 | 18.73 |
| ATOM | 1146 | C | GLY | A | 176 | 84.541 | 39.492 | 18.193 | 1.00 | 18.75 |
| ATOM | 1147 | O | GLY | A | 176 | 84.909 | 40.614 | 17.844 | 1.00 | 16.75 |
| ATOM | 1148 | N | ILE | A | 177 | 83.844 | 38.675 | 17.400 | 1.00 | 18.95 |
| ATOM | 1149 | CA | ILE | A | 177 | 83.459 | 39.023 | 16.023 | 1.00 | 17.03 |
| ATOM | 1150 | CB | ILE | A | 177 | 83.391 | 37.750 | 15.144 | 1.00 | 18.98 |
| ATOM | 1151 | CG2 | ILE | A | 177 | 82.803 | 38.078 | 13.767 | 1.00 | 20.31 |
| ATOM | 1152 | CG1 | ILE | A | 177 | 84.770 | 37.106 | 15.031 | 1.00 | 16.05 |
| ATOM | 1153 | CD1 | ILE | A | 177 | 84.720 | 35.603 | 14.862 | 1.00 | 17.66 |
| ATOM | 1154 | C | ILE | A | 177 | 82.107 | 39.713 | 15.933 | 1.00 | 13.93 |
| ATOM | 1155 | O | ILE | A | 177 | 81.087 | 39.148 | 16.280 | 1.00 | 16.03 |
| ATOM | 1156 | N | CYS | A | 178 | 82.107 | 40.917 | 15.407 | 1.00 | 12.55 |
| ATOM | 1157 | CA | CYS | A | 178 | 80.887 | 41.662 | 15.204 | 1.00 | 13.12 |
| ATOM | 1158 | CB | CYS | A | 178 | 81.095 | 43.115 | 15.644 | 1.00 | 12.26 |
| ATOM | 1159 | SG | CYS | A | 178 | 79.577 | 44.043 | 15.818 | 1.00 | 21.65 |
| ATOM | 1160 | C | CYS | A | 178 | 80.521 | 41.604 | 13.699 | 1.00 | 15.33 |
| ATOM | 1161 | O | CYS | A | 178 | 81.362 | 41.910 | 12.845 | 1.00 | 17.46 |
| ATOM | 1162 | N | HIS | A | 179 | 79.294 | 41.183 | 13.409 | 1.00 | 14.88 |
| ATOM | 1163 | CA | HIS | A | 179 | 78.779 | 41.037 | 12.050 | 1.00 | 18.49 |
| ATOM | 1164 | CB | HIS | A | 179 | 77.380 | 40.418 | 12.066 | 1.00 | 16.51 |
| ATOM | 1165 | CG | HIS | A | 179 | 76.978 | 39.835 | 10.736 | 1.00 | 19.21 |
| ATOM | 1166 | CD2 | HIS | A | 179 | 77.365 | 38.698 | 10.109 | 1.00 | 18.67 |
| ATOM | 1167 | ND1 | HIS | A | 179 | 76.083 | 40.442 | 9.882 | 1.00 | 16.97 |
| ATOM | 1168 | CE1 | HIS | A | 179 | 75.938 | 39.711 | 8.799 | 1.00 | 13.99 |
| ATOM | 1169 | NE2 | HIS | A | 179 | 76.705 | 38.649 | 8.912 | 1.00 | 15.08 |
| ATOM | 1170 | C | HIS | A | 179 | 78.767 | 42.383 | 11.325 | 1.00 | 20.60 |
| ATOM | 1171 | O | HIS | A | 179 | 79.474 | 42.552 | 10.338 | 1.00 | 17.90 |
| ATOM | 1172 | N | ARG | A | 180 | 77.980 | 43.331 | 11.875 | 1.00 | 19.78 |
| ATOM | 1173 | CA | ARG | A | 180 | 77.862 | 44.697 | 11.369 | 1.00 | 19.72 |
| ATOM | 1174 | CB | ARG | A | 180 | 79.237 | 45.298 | 11.122 | 1.00 | 22.50 |
| ATOM | 1175 | CG | ARG | A | 180 | 80.046 | 45.492 | 12.395 | 1.00 | 20.08 |
| ATOM | 1176 | CD | ARG | A | 180 | 81.157 | 46.504 | 12.195 | 1.00 | 20.28 |
| ATOM | 1177 | NE | ARG | A | 180 | 82.159 | 46.023 | 11.247 | 1.00 | 22.10 |
| ATOM | 1178 | CZ | ARG | A | 180 | 83.174 | 46.749 | 10.797 | 1.00 | 19.60 |
| ATOM | 1179 | NH1 | ARG | A | 180 | 83.369 | 47.979 | 11.242 | 1.00 | 20.78 |
| ATOM | 1180 | NH2 | ARG | A | 180 | 84.005 | 46.229 | 9.907 | 1.00 | 20.16 |
| ATOM | 1181 | C | ARG | A | 180 | 76.971 | 44.837 | 10.121 | 1.00 | 19.93 |
| ATOM | 1182 | O | ARG | A | 180 | 76.937 | 45.907 | 9.506 | 1.00 | 18.32 |
| ATOM | 1183 | N | ASP | A | 181 | 76.218 | 43.793 | 9.784 | 1.00 | 20.47 |
| ATOM | 1184 | CA | ASP | A | 181 | 75.305 | 43.846 | 8.640 | 1.00 | 22.91 |
| ATOM | 1185 | CB | ASP | A | 181 | 76.048 | 43.534 | 7.319 | 1.00 | 20.92 |
| ATOM | 1186 | CG | ASP | A | 181 | 75.296 | 43.997 | 6.056 | 1.00 | 24.19 |
| ATOM | 1187 | OD1 | ASP | A | 181 | 74.353 | 44.814 | 6.170 | 1.00 | 20.69 |
| ATOM | 1188 | OD2 | ASP | A | 181 | 75.663 | 43.529 | 4.948 | 1.00 | 22.69 |
| ATOM | 1189 | C | ASP | A | 181 | 74.106 | 42.930 | 8.853 | 1.00 | 22.26 |
| ATOM | 1190 | O | ASP | A | 181 | 73.738 | 42.132 | 7.980 | 1.00 | 22.03 |
| ATOM | 1191 | N | ILE | A | 182 | 73.486 | 43.061 | 10.041 | 1.00 | 21.81 |
| ATOM | 1192 | CA | ILE | A | 182 | 72.330 | 42.277 | 10.419 | 1.00 | 17.10 |
| ATOM | 1193 | CB | ILE | A | 182 | 72.215 | 42.135 | 11.959 | 1.00 | 18.31 |
| ATOM | 1194 | CG2 | ILE | A | 182 | 70.918 | 41.438 | 12.353 | 1.00 | 15.65 |
| ATOM | 1195 | CG1 | ILE | A | 182 | 73.431 | 41.377 | 12.489 | 1.00 | 16.67 |
| ATOM | 1196 | CD1 | ILE | A | 182 | 73.369 | 39.879 | 12.244 | 1.00 | 15.69 |
| ATOM | 1197 | C | ILE | A | 182 | 71.056 | 42.859 | 9.841 | 1.00 | 17.49 |
| ATOM | 1198 | O | ILE | A | 182 | 70.618 | 43.954 | 10.222 | 1.00 | 17.73 |
| ATOM | 1199 | N | LYS | A | 183 | 70.482 | 42.115 | 8.903 | 1.00 | 14.95 |
| ATOM | 1200 | CA | LYS | A | 183 | 69.273 | 42.519 | 8.220 | 1.00 | 12.48 |
| ATOM | 1201 | CB | LYS | A | 183 | 69.614 | 43.479 | 7.071 | 1.00 | 17.35 |
| ATOM | 1202 | CG | LYS | A | 183 | 70.456 | 42.850 | 5.959 | 1.00 | 14.20 |
| ATOM | 1203 | CD | LYS | A | 183 | 71.084 | 43.921 | 5.087 | 1.00 | 17.19 |
| ATOM | 1204 | CE | LYS | A | 183 | 71.955 | 43.310 | 4.001 | 1.00 | 20.09 |
| ATOM | 1205 | NZ | LYS | A | 183 | 72.604 | 44.349 | 3.158 | 1.00 | 17.13 |
| ATOM | 1206 | C | LYS | A | 183 | 68.548 | 41.288 | 7.705 | 1.00 | 8.04 |
| ATOM | 1207 | O | LYS | A | 183 | 69.132 | 40.220 | 7.644 | 1.00 | 8.53 |
| ATOM | 1208 | N | PRO | A | 184 | 67.270 | 41.414 | 7.360 | 1.00 | 7.13 |
| ATOM | 1209 | CD | PRO | A | 184 | 66.505 | 42.647 | 7.427 | 1.00 | 6.34 |
| ATOM | 1210 | CA | PRO | A | 184 | 66.460 | 40.292 | 6.884 | 1.00 | 11.96 |
| ATOM | 1211 | CB | PRO | A | 184 | 65.121 | 40.928 | 6.544 | 1.00 | 10.86 |
| ATOM | 1212 | CG | PRO | A | 184 | 65.096 | 42.183 | 7.334 | 1.00 | 8.34 |
| ATOM | 1213 | C | PRO | A | 184 | 67.030 | 39.540 | 5.668 | 1.00 | 14.64 |
| ATOM | 1214 | O | PRO | A | 184 | 66.792 | 38.343 | 5.527 | 1.00 | 18.32 |
| ATOM | 1215 | N | GLN | A | 185 | 67.771 | 40.226 | 4.788 | 1.00 | 17.59 |
| ATOM | 1216 | CA | GLN | A | 185 | 68.319 | 39.545 | 3.610 | 1.00 | 19.45 |
| ATOM | 1217 | CB | GLN | A | 185 | 68.582 | 40.495 | 2.431 | 1.00 | 22.98 |
| ATOM | 1218 | CG | GLN | A | 185 | 69.181 | 41.826 | 2.809 | 1.00 | 28.27 |
| ATOM | 1219 | CD | GLN | A | 185 | 68.121 | 42.897 | 2.974 | 1.00 | 32.07 |
| ATOM | 1220 | OE1 | GLN | A | 185 | 67.725 | 43.533 | 1.993 | 1.00 | 32.64 |
| ATOM | 1221 | NE2 | GLN | A | 185 | 67.657 | 43.105 | 4.223 | 1.00 | 26.42 |
| ATOM | 1222 | C | GLN | A | 185 | 69.544 | 38.734 | 3.938 | 1.00 | 19.77 |
| ATOM | 1223 | O | GLN | A | 185 | 69.951 | 37.860 | 3.150 | 1.00 | 16.26 |
| ATOM | 1224 | N | ASN | A | 186 | 70.135 | 38.997 | 5.117 | 1.00 | 17.25 |
| ATOM | 1225 | CA | ASN | A | 186 | 71.311 | 38.244 | 5.532 | 1.00 | 15.26 |
| ATOM | 1226 | CB | ASN | A | 186 | 72.408 | 39.145 | 6.120 | 1.00 | 17.32 |
| ATOM | 1227 | CG | ASN | A | 186 | 73.194 | 39.859 | 5.019 | 1.00 | 19.75 |
| ATOM | 1228 | OD1 | ASN | A | 186 | 73.140 | 39.464 | 3.844 | 1.00 | 19.21 |
| ATOM | 1229 | ND2 | ASN | A | 186 | 73.931 | 40.899 | 5.376 | 1.00 | 16.18 |
| ATOM | 1230 | C | ASN | A | 186 | 70.942 | 37.030 | 6.401 | 1.00 | 13.78 |
| ATOM | 1231 | O | ASN | A | 186 | 71.800 | 36.359 | 6.966 | 1.00 | 14.79 |
| ATOM | 1232 | N | LEU | A | 187 | 69.658 | 36.745 | 6.447 | 1.00 | 14.07 |
| ATOM | 1233 | CA | LEU | A | 187 | 69.098 | 35.618 | 7.182 | 1.00 | 16.11 |
| ATOM | 1234 | CB | LEU | A | 187 | 68.023 | 36.114 | 8.160 | 1.00 | 13.53 |
| ATOM | 1235 | CG | LEU | A | 187 | 68.465 | 37.246 | 9.086 | 1.00 | 14.77 |
| ATOM | 1236 | CD1 | LEU | A | 187 | 67.284 | 37.829 | 9.849 | 1.00 | 12.78 |
| ATOM | 1237 | CD2 | LEU | A | 187 | 69.573 | 36.763 | 10.004 | 1.00 | 14.06 |
| ATOM | 1238 | C | LEU | A | 187 | 68.490 | 34.600 | 6.219 | 1.00 | 18.88 |
| ATOM | 1239 | O | LEU | A | 187 | 67.385 | 34.788 | 5.712 | 1.00 | 20.91 |
| ATOM | 1240 | N | LEU | A | 188 | 69.224 | 33.529 | 5.972 | 1.00 | 21.65 |
| ATOM | 1241 | CA | LEU | A | 188 | 68.787 | 32.479 | 5.069 | 1.00 | 24.39 |
| ATOM | 1242 | CB | LEU | A | 188 | 70.000 | 31.759 | 4.474 | 1.00 | 24.36 |
| ATOM | 1243 | CG | LEU | A | 188 | 71.063 | 32.656 | 3.853 | 1.00 | 23.20 |
| ATOM | 1244 | CD1 | LEU | A | 188 | 72.240 | 31.834 | 3.352 | 1.00 | 22.15 |
| ATOM | 1245 | CD2 | LEU | A | 188 | 70.467 | 33.499 | 2.737 | 1.00 | 22.86 |
| ATOM | 1246 | C | LEU | A | 188 | 67.869 | 31.481 | 5.753 | 1.00 | 26.90 |
| ATOM | 1247 | O | LEU | A | 188 | 68.136 | 31.039 | 6.879 | 1.00 | 27.46 |
| ATOM | 1248 | N | LEU | A | 189 | 66.786 | 31.135 | 5.058 | 1.00 | 27.05 |
| ATOM | 1249 | CA | LEU | A | 189 | 65.802 | 30.200 | 5.559 | 1.00 | 29.15 |
| ATOM | 1250 | CB | LEU | A | 189 | 64.434 | 30.877 | 5.628 | 1.00 | 28.90 |
| ATOM | 1251 | CG | LEU | A | 189 | 64.289 | 32.054 | 6.581 | 1.00 | 26.77 |
| ATOM | 1252 | CD1 | LEU | A | 189 | 62.832 | 32.520 | 6.637 | 1.00 | 28.02 |
| ATOM | 1253 | CD2 | LEU | A | 189 | 64.798 | 31.679 | 7.971 | 1.00 | 27.53 |
| ATOM | 1254 | C | LEU | A | 189 | 65.668 | 28.977 | 4.666 | 1.00 | 33.15 |
| ATOM | 1255 | O | LEU | A | 189 | 65.827 | 29.059 | 3.435 | 1.00 | 33.46 |
| ATOM | 1256 | N | ASP | A | 190 | 65.376 | 27.857 | 5.327 | 1.00 | 35.73 |
| ATOM | 1257 | CA | ASP | A | 190 | 65.108 | 26.609 | 4.632 | 1.00 | 37.59 |
| ATOM | 1258 | CB | ASP | A | 190 | 65.798 | 25.424 | 5.338 | 1.00 | 38.82 |
| ATOM | 1259 | CG | ASP | A | 190 | 65.775 | 24.136 | 4.535 | 1.00 | 39.75 |
| ATOM | 1260 | OD1 | ASP | A | 190 | 66.831 | 23.515 | 4.370 | 1.00 | 39.88 |
| ATOM | 1261 | OD2 | ASP | A | 190 | 64.679 | 23.750 | 4.076 | 1.00 | 39.72 |
| ATOM | 1262 | C | ASP | A | 190 | 63.601 | 26.498 | 4.600 | 1.00 | 39.63 |
| ATOM | 1263 | O | ASP | A | 190 | 62.945 | 26.557 | 5.634 | 1.00 | 38.67 |
| ATOM | 1264 | N | PRO | A | 191 | 63.054 | 26.347 | 3.345 | 1.00 | 41.30 |
| ATOM | 1265 | CD | PRO | A | 191 | 63.606 | 26.905 | 2.106 | 1.00 | 43.05 |
| ATOM | 1266 | CA | PRO | A | 191 | 61.636 | 26.378 | 3.230 | 1.00 | 42.88 |
| ATOM | 1267 | CB | PRO | A | 191 | 61.355 | 25.989 | 1.827 | 1.00 | 43.47 |
| ATOM | 1268 | CG | PRO | A | 191 | 62.479 | 26.714 | 1.120 | 1.00 | 44.65 |
| ATOM | 1269 | C | PRO | A | 191 | 61.091 | 25.558 | 4.271 | 1.00 | 43.67 |
| ATOM | 1270 | O | PRO | A | 191 | 60.198 | 25.877 | 5.043 | 1.00 | 45.02 |
| ATOM | 1271 | N | ASP | A | 192 | 61.712 | 24.392 | 4.198 | 1.00 | 43.63 |
| ATOM | 1272 | CA | ASP | A | 192 | 61.232 | 23.245 | 4.931 | 1.00 | 43.69 |
| ATOM | 1273 | CB | ASP | A | 192 | 61.357 | 22.022 | 4.086 | 1.00 | 45.58 |
| ATOM | 1274 | CG | ASP | A | 192 | 60.818 | 22.195 | 2.695 | 1.00 | 46.33 |
| ATOM | 1275 | OD1 | ASP | A | 192 | 59.591 | 22.363 | 2.541 | 1.00 | 46.49 |
| ATOM | 1276 | OD2 | ASP | A | 192 | 61.616 | 22.168 | 1.743 | 1.00 | 48.09 |
| ATOM | 1277 | C | ASP | A | 192 | 61.792 | 22.660 | 6.271 | 1.00 | 42.33 |
| ATOM | 1278 | O | ASP | A | 192 | 61.192 | 21.674 | 6.684 | 1.00 | 43.45 |
| ATOM | 1279 | N | THR | A | 193 | 62.857 | 23.095 | 6.969 | 1.00 | 40.04 |
| ATOM | 1280 | CA | THR | A | 193 | 63.208 | 22.534 | 8.269 | 1.00 | 38.45 |
| ATOM | 1281 | CB | THR | A | 193 | 64.684 | 22.092 | 8.356 | 1.00 | 37.80 |
| ATOM | 1282 | OG1 | THR | A | 193 | 65.498 | 23.047 | 7.662 | 1.00 | 38.94 |
| ATOM | 1283 | CG2 | THR | A | 193 | 64.855 | 20.721 | 7.717 | 1.00 | 37.62 |
| ATOM | 1284 | C | THR | A | 193 | 62.899 | 23.654 | 9.248 | 1.00 | 37.60 |
| ATOM | 1285 | O | THR | A | 193 | 62.720 | 23.448 | 10.444 | 1.00 | 37.52 |
| ATOM | 1286 | N | ALA | A | 194 | 62.858 | 24.855 | 8.720 | 1.00 | 35.57 |
| ATOM | 1287 | CA | ALA | A | 194 | 62.599 | 26.006 | 9.539 | 1.00 | 34.21 |
| ATOM | 1288 | CB | ALA | A | 194 | 61.615 | 25.681 | 10.649 | 1.00 | 36.13 |
| ATOM | 1289 | C | ALA | A | 194 | 63.940 | 26.493 | 10.127 | 1.00 | 32.61 |
| ATOM | 1290 | O | ALA | A | 194 | 63.973 | 27.376 | 10.993 | 1.00 | 32.94 |
| ATOM | 1291 | N | VAL | A | 195 | 65.042 | 25.920 | 9.636 | 1.00 | 29.19 |
| ATOM | 1292 | CA | VAL | A | 195 | 66.376 | 26.298 | 10.094 | 1.00 | 25.71 |
| ATOM | 1293 | CB | VAL | A | 195 | 67.415 | 25.237 | 9.725 | 1.00 | 23.91 |
| ATOM | 1294 | CG1 | VAL | A | 195 | 68.812 | 25.712 | 10.092 | 1.00 | 24.71 |
| ATOM | 1295 | CG2 | VAL | A | 195 | 67.104 | 23.922 | 10.420 | 1.00 | 24.73 |
| ATOM | 1296 | C | VAL | A | 195 | 66.791 | 27.640 | 9.486 | 1.00 | 26.18 |
| ATOM | 1297 | O | VAL | A | 195 | 66.454 | 27.934 | 8.332 | 1.00 | 27.87 |
| ATOM | 1298 | N | LEU | A | 196 | 67.516 | 28.447 | 10.271 | 1.00 | 21.67 |
| ATOM | 1299 | CA | LEU | A | 196 | 67.980 | 29.752 | 9.838 | 1.00 | 18.05 |
| ATOM | 1300 | CB | LEU | A | 196 | 67.348 | 30.885 | 10.662 | 1.00 | 17.64 |
| ATOM | 1301 | CG | LEU | A | 196 | 67.930 | 32.299 | 10.469 | 1.00 | 17.61 |
| ATOM | 1302 | CD1 | LEU | A | 196 | 66.867 | 33.352 | 10.714 | 1.00 | 16.49 |
| ATOM | 1303 | CD2 | LEU | A | 196 | 69.143 | 32.535 | 11.368 | 1.00 | 20.40 |
| ATOM | 1304 | C | LEU | A | 196 | 69.484 | 29.830 | 9.893 | 1.00 | 17.95 |
| ATOM | 1305 | O | LEU | A | 196 | 70.118 | 29.286 | 10.795 | 1.00 | 16.92 |
| ATOM | 1306 | N | LYS | A | 197 | 70.062 | 30.505 | 8.915 | 1.00 | 17.55 |
| ATOM | 1307 | CA | LYS | A | 197 | 71.496 | 30.653 | 8.856 | 1.00 | 17.81 |
| ATOM | 1308 | CB | LYS | A | 197 | 72.107 | 29.725 | 7.806 | 1.00 | 19.05 |
| ATOM | 1309 | CG | LYS | A | 197 | 71.753 | 28.267 | 8.014 | 1.00 | 21.12 |
| ATOM | 1310 | CD | LYS | A | 197 | 72.851 | 27.344 | 7.520 | 1.00 | 21.21 |
| ATOM | 1311 | CE | LYS | A | 197 | 72.434 | 25.880 | 7.619 | 1.00 | 22.57 |
| ATOM | 1312 | NZ | LYS | A | 197 | 73.276 | 25.109 | 8.585 | 1.00 | 26.08 |
| ATOM | 1313 | C | LYS | A | 197 | 71.849 | 32.095 | 8.570 | 1.00 | 18.79 |
| ATOM | 1314 | O | LYS | A | 197 | 71.160 | 32.778 | 7.795 | 1.00 | 20.26 |
| ATOM | 1315 | N | LEU | A | 198 | 72.922 | 32.548 | 9.204 | 1.00 | 18.11 |
| ATOM | 1316 | CA | LEU | A | 198 | 73.418 | 33.905 | 9.053 | 1.00 | 18.99 |
| ATOM | 1317 | CB | LEU | A | 198 | 74.203 | 34.298 | 10.327 | 1.00 | 19.76 |
| ATOM | 1318 | CG | LEU | A | 198 | 73.948 | 35.690 | 10.921 | 1.00 | 19.89 |
| ATOM | 1319 | CD1 | LEU | A | 198 | 75.165 | 36.165 | 11.692 | 1.00 | 16.57 |
| ATOM | 1320 | CD2 | LEU | A | 198 | 73.562 | 36.705 | 9.850 | 1.00 | 14.92 |
| ATOM | 1321 | C | LEU | A | 198 | 74.391 | 33.921 | 7.883 | 1.00 | 17.30 |
| ATOM | 1322 | O | LEU | A | 198 | 75.208 | 33.015 | 7.757 | 1.00 | 15.58 |
| ATOM | 1323 | N | CYS | A | 199 | 74.331 | 34.947 | 7.050 | 1.00 | 15.58 |
| ATOM | 1324 | CA | CYS | A | 199 | 75.272 | 35.004 | 5.938 | 1.00 | 17.83 |
| ATOM | 1325 | CB | CYS | A | 199 | 74.615 | 34.546 | 4.613 | 1.00 | 14.27 |
| ATOM | 1326 | SG | CYS | A | 199 | 73.261 | 35.610 | 4.074 | 1.00 | 19.47 |
| ATOM | 1327 | C | CYS | A | 199 | 75.868 | 36.383 | 5.789 | 1.00 | 17.30 |
| ATOM | 1328 | O | CYS | A | 199 | 75.511 | 37.324 | 6.532 | 1.00 | 19.26 |
| ATOM | 1329 | N | ASP | A | 200 | 76.758 | 36.488 | 4.798 | 1.00 | 15.75 |
| ATOM | 1330 | CA | ASP | A | 200 | 77.438 | 37.708 | 4.426 | 1.00 | 13.20 |
| ATOM | 1331 | CB | ASP | A | 200 | 76.467 | 38.705 | 3.819 | 1.00 | 10.32 |
| ATOM | 1332 | CG | ASP | A | 200 | 77.134 | 39.973 | 3.349 | 1.00 | 6.94 |
| ATOM | 1333 | OD1 | ASP | A | 200 | 76.421 | 40.968 | 3.160 | 1.00 | 17.09 |
| ATOM | 1334 | OD2 | ASP | A | 200 | 78.361 | 39.977 | 3.177 | 1.00 | 15.72 |
| ATOM | 1335 | C | ASP | A | 200 | 78.272 | 38.329 | 5.543 | 1.00 | 17.58 |
| ATOM | 1336 | O | ASP | A | 200 | 77.822 | 39.222 | 6.256 | 1.00 | 17.88 |
| ATOM | 1337 | N | PHE | A | 201 | 79.503 | 37.869 | 5.633 | 1.00 | 17.55 |
| ATOM | 1338 | CA | PHE | A | 201 | 80.459 | 38.349 | 6.598 | 1.00 | 20.60 |
| ATOM | 1339 | CB | PHE | A | 201 | 81.208 | 37.152 | 7.212 | 1.00 | 20.19 |
| ATOM | 1340 | CG | PHE | A | 201 | 80.333 | 36.352 | 8.151 | 1.00 | 20.68 |
| ATOM | 1341 | CD1 | PHE | A | 201 | 79.331 | 35.537 | 7.661 | 1.00 | 18.20 |
| ATOM | 1342 | CD2 | PHE | A | 201 | 80.497 | 36.449 | 9.536 | 1.00 | 20.32 |
| ATOM | 1343 | CE1 | PHE | A | 201 | 78.501 | 34.831 | 8.517 | 1.00 | 21.23 |
| ATOM | 1344 | CE2 | PHE | A | 201 | 79.677 | 35.742 | 10.395 | 1.00 | 19.54 |
| ATOM | 1345 | CZ | PHE | A | 201 | 78.674 | 34.934 | 9.891 | 1.00 | 20.02 |
| ATOM | 1346 | C | PHE | A | 201 | 81.422 | 39.373 | 5.986 | 1.00 | 20.61 |
| ATOM | 1347 | O | PHE | A | 201 | 82.518 | 39.578 | 6.498 | 1.00 | 21.05 |
| ATOM | 1348 | N | GLY | A | 202 | 80.987 | 40.031 | 4.893 | 1.00 | 22.55 |
| ATOM | 1349 | CA | GLY | A | 202 | 81.808 | 41.041 | 4.226 | 1.00 | 21.39 |
| ATOM | 1350 | C | GLY | A | 202 | 81.992 | 42.289 | 5.059 | 1.00 | 21.03 |
| ATOM | 1351 | O | GLY | A | 202 | 82.936 | 43.038 | 4.873 | 1.00 | 19.70 |
| ATOM | 1352 | N | SER | A | 203 | 81.097 | 42.499 | 6.003 | 1.00 | 23.29 |
| ATOM | 1353 | CA | SER | A | 203 | 81.179 | 43.643 | 6.897 | 1.00 | 24.09 |
| ATOM | 1354 | CB | SER | A | 203 | 79.785 | 44.251 | 7.056 | 1.00 | 24.42 |
| ATOM | 1355 | OG | SER | A | 203 | 79.469 | 45.139 | 6.002 | 1.00 | 25.73 |
| ATOM | 1356 | C | SER | A | 203 | 81.696 | 43.202 | 8.290 | 1.00 | 23.68 |
| ATOM | 1357 | O | SER | A | 203 | 81.769 | 44.013 | 9.209 | 1.00 | 26.83 |
| ATOM | 1358 | N | ALA | A | 204 | 81.993 | 41.910 | 8.442 | 1.00 | 23.06 |
| ATOM | 1359 | CA | ALA | A | 204 | 82.435 | 41.321 | 9.722 | 1.00 | 23.57 |
| ATOM | 1360 | CB | ALA | A | 204 | 82.359 | 39.806 | 9.679 | 1.00 | 21.25 |
| ATOM | 1361 | C | ALA | A | 204 | 83.812 | 41.774 | 10.170 | 1.00 | 23.86 |
| ATOM | 1362 | O | ALA | A | 204 | 84.722 | 41.929 | 9.364 | 1.00 | 25.34 |
| ATOM | 1363 | N | LYS | A | 205 | 83.957 | 42.001 | 11.477 | 1.00 | 25.35 |
| ATOM | 1364 | CA | LYS | A | 205 | 85.227 | 42.468 | 12.029 | 1.00 | 25.22 |
| ATOM | 1365 | CB | LYS | A | 205 | 85.278 | 43.999 | 11.925 | 1.00 | 22.62 |
| ATOM | 1366 | CG | LYS | A | 205 | 86.640 | 44.627 | 12.196 | 1.00 | 23.08 |
| ATOM | 1367 | CD | LYS | A | 205 | 86.596 | 46.131 | 11.957 | 1.00 | 24.63 |
| ATOM | 1368 | CE | LYS | A | 205 | 87.502 | 46.906 | 12.902 | 1.00 | 25.30 |
| ATOM | 1369 | NZ | LYS | A | 205 | 88.938 | 46.568 | 12.721 | 1.00 | 27.91 |
| ATOM | 1370 | C | LYS | A | 205 | 85.413 | 42.062 | 13.488 | 1.00 | 26.41 |
| ATOM | 1371 | O | LYS | A | 205 | 84.499 | 42.145 | 14.298 | 1.00 | 26.55 |
| ATOM | 1372 | N | GLN | A | 206 | 86.628 | 41.698 | 13.830 | 1.00 | 28.82 |
| ATOM | 1373 | CA | GLN | A | 206 | 86.934 | 41.382 | 15.205 | 1.00 | 29.31 |
| ATOM | 1374 | CB | GLN | A | 206 | 88.113 | 40.422 | 15.285 | 1.00 | 32.58 |
| ATOM | 1375 | CG | GLN | A | 206 | 87.965 | 39.376 | 16.374 | 1.00 | 37.25 |
| ATOM | 1376 | CD | GLN | A | 206 | 89.190 | 38.506 | 16.507 | 1.00 | 40.73 |
| ATOM | 1377 | OE1 | GLN | A | 206 | 90.207 | 38.912 | 17.088 | 1.00 | 43.91 |
| ATOM | 1378 | NE2 | GLN | A | 206 | 89.105 | 37.300 | 15.965 | 1.00 | 43.70 |
| ATOM | 1379 | C | GLN | A | 206 | 87.284 | 42.701 | 15.882 | 1.00 | 26.36 |
| ATOM | 1380 | O | GLN | A | 206 | 88.311 | 43.291 | 15.588 | 1.00 | 25.43 |
| ATOM | 1381 | N | LEU | A | 207 | 86.410 | 43.193 | 16.746 | 1.00 | 26.35 |
| ATOM | 1382 | CA | LEU | A | 207 | 86.677 | 44.474 | 17.404 | 1.00 | 26.99 |
| ATOM | 1383 | CB | LEU | A | 207 | 85.415 | 45.060 | 18.048 | 1.00 | 26.08 |
| ATOM | 1384 | CG | LEU | A | 207 | 84.203 | 45.273 | 17.119 | 1.00 | 29.98 |
| ATOM | 1385 | CD1 | LEU | A | 207 | 83.250 | 46.312 | 17.693 | 1.00 | 27.89 |
| ATOM | 1386 | CD2 | LEU | A | 207 | 84.637 | 45.683 | 15.719 | 1.00 | 28.11 |
| ATOM | 1387 | C | LEU | A | 207 | 87.845 | 44.359 | 18.396 | 1.00 | 26.56 |
| ATOM | 1388 | O | LEU | A | 207 | 87.995 | 43.337 | 19.067 | 1.00 | 27.11 |
| ATOM | 1389 | N | VAL | A | 208 | 88.684 | 45.399 | 18.448 | 1.00 | 27.33 |
| ATOM | 1390 | CA | VAL | A | 208 | 89.852 | 45.436 | 19.332 | 1.00 | 29.22 |
| ATOM | 1391 | CB | VAL | A | 208 | 91.197 | 45.028 | 18.665 | 1.00 | 30.94 |
| ATOM | 1392 | CG1 | VAL | A | 208 | 90.973 | 44.129 | 17.448 | 1.00 | 31.76 |
| ATOM | 1393 | CG2 | VAL | A | 208 | 92.055 | 46.232 | 18.314 | 1.00 | 31.13 |
| ATOM | 1394 | C | VAL | A | 208 | 89.948 | 46.776 | 20.063 | 1.00 | 29.36 |
| ATOM | 1395 | O | VAL | A | 208 | 89.664 | 47.830 | 19.483 | 1.00 | 27.20 |
| ATOM | 1396 | N | ARG | A | 209 | 90.295 | 46.679 | 21.365 | 1.00 | 30.74 |
| ATOM | 1397 | CA | ARG | A | 209 | 90.397 | 47.813 | 22.319 | 1.00 | 30.09 |
| ATOM | 1398 | CB | ARG | A | 209 | 91.437 | 47.498 | 23.408 | 1.00 | 30.05 |
| ATOM | 1399 | CG | ARG | A | 209 | 90.988 | 47.859 | 24.823 | 1.00 | 29.36 |
| ATOM | 1400 | CD | ARG | A | 209 | 90.443 | 49.276 | 24.922 | 1.00 | 23.91 |
| ATOM | 1401 | NE | ARG | A | 209 | 90.796 | 49.941 | 26.198 | 1.00 | 20.44 |
| ATOM | 1402 | CZ | ARG | A | 209 | 89.905 | 50.504 | 27.022 | 1.00 | 18.05 |
| ATOM | 1403 | NH1 | ARG | A | 209 | 88.609 | 50.282 | 26.872 | 1.00 | 11.51 |
| ATOM | 1404 | NH2 | ARG | A | 209 | 90.318 | 51.296 | 28.016 | 1.00 | 22.90 |
| ATOM | 1405 | C | ARG | A | 209 | 90.690 | 49.192 | 21.714 | 1.00 | 30.73 |
| ATOM | 1406 | O | ARG | A | 209 | 89.804 | 50.061 | 21.696 | 1.00 | 35.19 |
| ATOM | 1407 | N | GLY | A | 210 | 91.928 | 49.435 | 21.303 | 1.00 | 30.36 |
| ATOM | 1408 | CA | GLY | A | 210 | 92.268 | 50.767 | 20.800 | 1.00 | 31.43 |
| ATOM | 1409 | C | GLY | A | 210 | 92.263 | 50.913 | 19.282 | 1.00 | 31.78 |
| ATOM | 1410 | O | GLY | A | 210 | 93.049 | 51.685 | 18.732 | 1.00 | 30.36 |
| ATOM | 1411 | N | GLU | A | 211 | 91.381 | 50.184 | 18.615 | 1.00 | 30.86 |
| ATOM | 1412 | CA | GLU | A | 211 | 91.287 | 50.246 | 17.162 | 1.00 | 29.71 |
| ATOM | 1413 | CB | GLU | A | 211 | 91.453 | 48.851 | 16.576 | 1.00 | 31.43 |
| ATOM | 1414 | CG | GLU | A | 211 | 92.494 | 48.766 | 15.482 | 1.00 | 35.03 |
| ATOM | 1415 | CD | GLU | A | 211 | 92.396 | 47.475 | 14.708 | 1.00 | 36.69 |
| ATOM | 1416 | OE1 | GLU | A | 211 | 93.283 | 46.614 | 14.883 | 1.00 | 39.03 |
| ATOM | 1417 | OE2 | GLU | A | 211 | 91.427 | 47.319 | 13.936 | 1.00 | 37.36 |
| ATOM | 1418 | C | GLU | A | 211 | 89.958 | 50.842 | 16.715 | 1.00 | 25.76 |
| ATOM | 1419 | O | GLU | A | 211 | 88.919 | 50.217 | 16.864 | 1.00 | 26.05 |
| ATOM | 1420 | N | PRO | A | 212 | 89.980 | 52.052 | 16.146 | 1.00 | 23.98 |
| ATOM | 1421 | CD | PRO | A | 212 | 91.187 | 52.863 | 15.912 | 1.00 | 25.11 |
| ATOM | 1422 | CA | PRO | A | 212 | 88.766 | 52.713 | 15.662 | 1.00 | 25.74 |
| ATOM | 1423 | CB | PRO | A | 212 | 89.262 | 54.039 | 15.068 | 1.00 | 25.32 |
| ATOM | 1424 | CG | PRO | A | 212 | 90.636 | 54.232 | 15.615 | 1.00 | 26.73 |
| ATOM | 1425 | C | PRO | A | 212 | 88.067 | 51.890 | 14.576 | 1.00 | 24.55 |
| ATOM | 1426 | O | PRO | A | 212 | 88.708 | 51.170 | 13.811 | 1.00 | 23.81 |
| ATOM | 1427 | N | ASN | A | 213 | 86.756 | 52.012 | 14.530 | 1.00 | 23.69 |
| ATOM | 1428 | CA | ASN | A | 213 | 85.929 | 51.321 | 13.565 | 1.00 | 21.45 |
| ATOM | 1429 | CB | ASN | A | 213 | 85.036 | 50.272 | 14.240 | 1.00 | 21.67 |
| ATOM | 1430 | CG | ASN | A | 213 | 85.724 | 49.456 | 15.309 | 1.00 | 23.99 |
| ATOM | 1431 | OD1 | ASN | A | 213 | 86.444 | 48.509 | 15.011 | 1.00 | 22.92 |
| ATOM | 1432 | ND2 | ASN | A | 213 | 85.489 | 49.819 | 16.581 | 1.00 | 25.34 |
| ATOM | 1433 | C | ASN | A | 213 | 85.045 | 52.347 | 12.887 | 1.00 | 21.95 |
| ATOM | 1434 | O | ASN | A | 213 | 84.634 | 53.328 | 13.525 | 1.00 | 18.41 |
| ATOM | 1435 | N | VAL | A | 214 | 84.737 | 52.133 | 11.593 | 1.00 | 21.29 |
| ATOM | 1436 | CA | VAL | A | 214 | 83.878 | 53.079 | 10.875 | 1.00 | 18.80 |
| ATOM | 1437 | CB | VAL | A | 214 | 83.896 | 52.867 | 9.333 | 1.00 | 20.83 |
| ATOM | 1438 | CG1 | VAL | A | 214 | 85.286 | 53.089 | 8.776 | 1.00 | 18.29 |
| ATOM | 1439 | CG2 | VAL | A | 214 | 83.399 | 51.474 | 8.972 | 1.00 | 17.72 |
| ATOM | 1440 | C | VAL | A | 214 | 82.450 | 52.996 | 11.399 | 1.00 | 16.35 |
| ATOM | 1441 | O | VAL | A | 214 | 81.946 | 51.909 | 11.662 | 1.00 | 15.23 |
| ATOM | 1442 | N | SER | A | 215 | 81.819 | 54.152 | 11.565 | 1.00 | 17.03 |
| ATOM | 1443 | CA | SER | A | 215 | 80.451 | 54.244 | 12.092 | 1.00 | 17.65 |
| ATOM | 1444 | CB | SER | A | 215 | 80.234 | 55.609 | 12.752 | 1.00 | 17.01 |
| ATOM | 1445 | OG | SER | A | 215 | 81.119 | 56.581 | 12.212 | 1.00 | 20.75 |
| ATOM | 1446 | C | SER | A | 215 | 79.374 | 54.022 | 11.035 | 1.00 | 18.35 |
| ATOM | 1447 | O | SER | A | 215 | 78.277 | 53.570 | 11.349 | 1.00 | 14.87 |
| ATOM | 1448 | N | PTR | A | 216 | 79.699 | 54.329 | 9.785 | 1.00 | 19.96 |
| ATOM | 1449 | CA | PTR | A | 216 | 78.779 | 54.432 | 8.719 | 1.00 | 23.18 |
| ATOM | 1450 | CB | PTR | A | 216 | 78.923 | 55.195 | 7.630 | 1.00 | 24.24 |
| ATOM | 1451 | CG | PTR | A | 216 | 77.794 | 55.416 | 6.633 | 1.00 | 24.41 |
| ATOM | 1452 | CD1 | PTR | A | 216 | 76.957 | 56.513 | 6.749 | 1.00 | 23.89 |
| ATOM | 1453 | CE1 | PTR | A | 216 | 75.931 | 56.738 | 5.847 | 1.00 | 25.55 |
| ATOM | 1454 | CD2 | PTR | A | 216 | 77.573 | 54.531 | 5.571 | 1.00 | 24.95 |
| ATOM | 1455 | CE2 | PTR | A | 216 | 76.541 | 54.746 | 4.655 | 1.00 | 27.09 |
| ATOM | 1456 | CZ | PTR | A | 216 | 75.718 | 55.852 | 4.802 | 1.00 | 26.51 |
| ATOM | 1457 | OH | PTR | A | 216 | 74.692 | 56.094 | 3.906 | 1.00 | 23.68 |
| ATOM | 1458 | C | PTR | A | 216 | 78.697 | 52.759 | 8.113 | 1.00 | 22.70 |
| ATOM | 1459 | O | PTR | A | 216 | 79.483 | 52.545 | 7.136 | 1.00 | 23.40 |
| ATOM | 1460 | P | PTR | A | 216 | 73.386 | 55.246 | 3.955 | 1.00 | 25.02 |
| ATOM | 1461 | O1P | PTR | A | 216 | 72.673 | 55.580 | 2.615 | 1.00 | 26.72 |
| ATOM | 1462 | O2P | PTR | A | 216 | 72.550 | 55.844 | 5.109 | 1.00 | 24.99 |
| ATOM | 1463 | O3P | PTR | A | 216 | 73.633 | 53.789 | 4.116 | 1.00 | 26.79 |
| ATOM | 1464 | N | ILE | A | 217 | 78.412 | 51.825 | 9.040 | 1.00 | 22.92 |
| ATOM | 1465 | CA | ILE | A | 217 | 78.245 | 50.432 | 8.679 | 1.00 | 22.89 |
| ATOM | 1466 | CB | ILE | A | 217 | 79.151 | 49.511 | 9.482 | 1.00 | 24.36 |
| ATOM | 1467 | CG2 | ILE | A | 217 | 80.338 | 49.075 | 8.646 | 1.00 | 24.92 |
| ATOM | 1468 | CG1 | ILE | A | 217 | 79.620 | 50.215 | 10.755 | 1.00 | 26.42 |
| ATOM | 1469 | CD1 | ILE | A | 217 | 78.508 | 50.527 | 11.726 | 1.00 | 25.66 |
| ATOM | 1470 | C | ILE | A | 217 | 76.788 | 50.056 | 8.867 | 1.00 | 22.46 |
| ATOM | 1471 | O | ILE | A | 217 | 75.904 | 50.924 | 8.917 | 1.00 | 24.48 |
| ATOM | 1472 | N | CYS | A | 218 | 76.529 | 48.727 | 8.924 | 1.00 | 23.13 |
| ATOM | 1473 | CA | CYS | A | 218 | 75.205 | 48.111 | 9.040 | 1.00 | 24.75 |
| ATOM | 1474 | CB | CYS | A | 218 | 74.546 | 48.490 | 10.371 | 1.00 | 24.22 |
| ATOM | 1475 | SG | CYS | A | 218 | 75.331 | 47.761 | 11.851 | 1.00 | 27.07 |
| ATOM | 1476 | C | CYS | A | 218 | 74.348 | 48.458 | 7.777 | 1.00 | 25.17 |
| ATOM | 1477 | O | CYS | A | 218 | 74.899 | 48.971 | 6.801 | 1.00 | 30.86 |
| ATOM | 1478 | N | SER | A | 219 | 73.039 | 48.193 | 7.713 | 1.00 | 23.61 |
| ATOM | 1479 | CA | SER | A | 219 | 72.243 | 48.746 | 6.594 | 1.00 | 21.63 |
| ATOM | 1480 | CB | SER | A | 219 | 71.262 | 47.789 | 5.922 | 1.00 | 13.51 |
| ATOM | 1481 | OG | SER | A | 219 | 70.190 | 48.493 | 5.322 | 1.00 | 22.93 |
| ATOM | 1482 | C | SER | A | 219 | 71.469 | 49.806 | 7.342 | 1.00 | 22.86 |
| ATOM | 1483 | O | SER | A | 219 | 71.084 | 49.549 | 8.473 | 1.00 | 24.73 |
| ATOM | 1484 | N | ARG | A | 220 | 71.224 | 50.983 | 6.779 | 1.00 | 21.86 |
| ATOM | 1485 | CA | ARG | A | 220 | 70.550 | 52.102 | 7.471 | 1.00 | 22.08 |
| ATOM | 1486 | CB | ARG | A | 220 | 70.072 | 53.104 | 6.426 | 1.00 | 19.58 |
| ATOM | 1487 | CG | ARG | A | 220 | 69.553 | 54.403 | 7.038 | 1.00 | 19.73 |
| ATOM | 1488 | CD | ARG | A | 220 | 68.658 | 55.174 | 6.086 | 1.00 | 21.19 |
| ATOM | 1489 | NE | ARG | A | 220 | 69.359 | 55.647 | 4.880 | 1.00 | 19.24 |
| ATOM | 1490 | CZ | ARG | A | 220 | 68.811 | 55.754 | 3.687 | 1.00 | 22.72 |
| ATOM | 1491 | NH1 | ARG | A | 220 | 67.545 | 55.437 | 3.505 | 1.00 | 19.16 |
| ATOM | 1492 | NH2 | ARG | A | 220 | 69.533 | 56.174 | 2.652 | 1.00 | 19.72 |
| ATOM | 1493 | C | ARG | A | 220 | 69.395 | 51.797 | 8.423 | 1.00 | 22.38 |
| ATOM | 1494 | O | ARG | A | 220 | 69.378 | 52.292 | 9.540 | 1.00 | 25.19 |
| ATOM | 1495 | N | TYR | A | 221 | 68.431 | 50.981 | 8.002 | 1.00 | 20.53 |
| ATOM | 1496 | CA | TYR | A | 221 | 67.262 | 50.685 | 8.830 | 1.00 | 20.22 |
| ATOM | 1497 | CB | TYR | A | 221 | 66.239 | 49.867 | 8.042 | 1.00 | 21.19 |
| ATOM | 1498 | CG | TYR | A | 221 | 65.495 | 50.609 | 6.941 | 1.00 | 24.83 |
| ATOM | 1499 | CD1 | TYR | A | 221 | 64.514 | 49.960 | 6.207 | 1.00 | 24.53 |
| ATOM | 1500 | CE1 | TYR | A | 221 | 63.826 | 50.608 | 5.193 | 1.00 | 27.31 |
| ATOM | 1501 | CD2 | TYR | A | 221 | 65.773 | 51.938 | 6.629 | 1.00 | 24.67 |
| ATOM | 1502 | CE2 | TYR | A | 221 | 65.091 | 52.594 | 5.619 | 1.00 | 27.03 |
| ATOM | 1503 | CZ | TYR | A | 221 | 64.117 | 51.920 | 4.903 | 1.00 | 27.02 |
| ATOM | 1504 | OH | TYR | A | 221 | 63.421 | 52.568 | 3.907 | 1.00 | 27.95 |
| ATOM | 1505 | C | TYR | A | 221 | 67.605 | 49.936 | 10.144 | 1.00 | 19.96 |
| ATOM | 1506 | O | TYR | A | 221 | 66.871 | 50.021 | 11.121 | 1.00 | 17.56 |
| ATOM | 1507 | N | TYR | A | 222 | 68.704 | 49.203 | 10.115 | 1.00 | 16.92 |
| ATOM | 1508 | CA | TYR | A | 222 | 69.162 | 48.374 | 11.222 | 1.00 | 17.59 |
| ATOM | 1509 | CB | TYR | A | 222 | 69.437 | 46.955 | 10.655 | 1.00 | 16.55 |
| ATOM | 1510 | CG | TYR | A | 222 | 68.318 | 46.531 | 9.726 | 1.00 | 17.84 |
| ATOM | 1511 | CD1 | TYR | A | 222 | 67.135 | 46.032 | 10.241 | 1.00 | 15.19 |
| ATOM | 1512 | CE1 | TYR | A | 222 | 66.066 | 45.763 | 9.425 | 1.00 | 17.87 |
| ATOM | 1513 | CD2 | TYR | A | 222 | 68.407 | 46.741 | 8.336 | 1.00 | 17.61 |
| ATOM | 1514 | CE2 | TYR | A | 222 | 67.340 | 46.451 | 7.499 | 1.00 | 17.42 |
| ATOM | 1515 | CZ | TYR | A | 222 | 66.168 | 45.972 | 8.049 | 1.00 | 20.27 |
| ATOM | 1516 | OH | TYR | A | 222 | 65.076 | 45.732 | 7.251 | 1.00 | 17.86 |
| ATOM | 1517 | C | TYR | A | 222 | 70.410 | 48.952 | 11.885 | 1.00 | 14.52 |
| ATOM | 1518 | O | TYR | A | 222 | 71.144 | 48.242 | 12.546 | 1.00 | 18.29 |
| ATOM | 1519 | N | ARG | A | 223 | 70.659 | 50.231 | 11.670 | 1.00 | 13.60 |
| ATOM | 1520 | CA | ARG | A | 223 | 71.846 | 50.892 | 12.198 | 1.00 | 16.34 |
| ATOM | 1521 | CB | ARG | A | 223 | 72.176 | 52.051 | 11.286 | 1.00 | 14.55 |
| ATOM | 1522 | CG | ARG | A | 223 | 73.552 | 52.627 | 11.459 | 1.00 | 14.37 |
| ATOM | 1523 | CD | ARG | A | 223 | 73.863 | 53.596 | 10.318 | 1.00 | 19.59 |
| ATOM | 1524 | NE | ARG | A | 223 | 74.359 | 52.892 | 9.127 | 1.00 | 18.67 |
| ATOM | 1525 | CZ | ARG | A | 223 | 74.179 | 53.327 | 7.859 | 1.00 | 20.50 |
| ATOM | 1526 | NH1 | ARG | A | 223 | 73.492 | 54.430 | 7.613 | 1.00 | 14.45 |
| ATOM | 1527 | NH2 | ARG | A | 223 | 74.693 | 52.643 | 6.844 | 1.00 | 19.01 |
| ATOM | 1528 | C | ARG | A | 223 | 71.647 | 51.417 | 13.633 | 1.00 | 16.61 |
| ATOM | 1529 | O | ARG | A | 223 | 70.656 | 52.093 | 13.917 | 1.00 | 17.43 |
| ATOM | 1530 | N | ALA | A | 224 | 72.609 | 51.109 | 14.506 | 1.00 | 18.18 |
| ATOM | 1531 | CA | ALA | A | 224 | 72.582 | 51.543 | 15.927 | 1.00 | 18.38 |
| ATOM | 1532 | CB | ALA | A | 224 | 73.711 | 50.884 | 16.686 | 1.00 | 14.89 |
| ATOM | 1533 | C | ALA | A | 224 | 72.704 | 53.055 | 16.042 | 1.00 | 20.36 |
| ATOM | 1534 | O | ALA | A | 224 | 73.455 | 53.686 | 15.289 | 1.00 | 22.22 |
| ATOM | 1535 | N | PRO | A | 225 | 71.985 | 53.669 | 16.994 | 1.00 | 21.61 |
| ATOM | 1536 | CD | PRO | A | 225 | 71.087 | 52.998 | 17.959 | 1.00 | 23.19 |
| ATOM | 1537 | CA | PRO | A | 225 | 72.028 | 55.114 | 17.194 | 1.00 | 20.61 |
| ATOM | 1538 | CB | PRO | A | 225 | 71.154 | 55.367 | 18.446 | 1.00 | 23.43 |
| ATOM | 1539 | CG | PRO | A | 225 | 70.894 | 54.023 | 19.050 | 1.00 | 20.97 |
| ATOM | 1540 | C | PRO | A | 225 | 73.437 | 55.664 | 17.383 | 1.00 | 19.98 |
| ATOM | 1541 | O | PRO | A | 225 | 73.749 | 56.744 | 16.870 | 1.00 | 21.76 |
| ATOM | 1542 | N | GLU | A | 226 | 74.297 | 54.942 | 18.109 | 1.00 | 17.57 |
| ATOM | 1543 | CA | GLU | A | 226 | 75.675 | 55.421 | 18.336 | 1.00 | 18.64 |
| ATOM | 1544 | CB | GLU | A | 226 | 76.463 | 54.477 | 19.247 | 1.00 | 18.48 |
| ATOM | 1545 | CG | GLU | A | 226 | 75.604 | 53.678 | 20.196 | 1.00 | 21.27 |
| ATOM | 1546 | CD | GLU | A | 226 | 75.033 | 52.420 | 19.594 | 1.00 | 14.40 |
| ATOM | 1547 | OE1 | GLU | A | 226 | 73.840 | 52.402 | 19.337 | 1.00 | 18.85 |
| ATOM | 1548 | OE2 | GLU | A | 226 | 75.766 | 51.443 | 19.432 | 1.00 | 18.95 |
| ATOM | 1549 | C | GLU | A | 226 | 76.430 | 55.578 | 17.018 | 1.00 | 17.88 |
| ATOM | 1550 | O | GLU | A | 226 | 77.238 | 56.487 | 16.856 | 1.00 | 16.85 |
| ATOM | 1551 | N | LEU | A | 227 | 76.155 | 54.676 | 16.078 | 1.00 | 17.24 |
| ATOM | 1552 | CA | LEU | A | 227 | 76.799 | 54.723 | 14.761 | 1.00 | 15.64 |
| ATOM | 1553 | CB | LEU | A | 227 | 76.282 | 53.558 | 13.910 | 1.00 | 12.39 |
| ATOM | 1554 | CG | LEU | A | 227 | 77.119 | 52.296 | 13.831 | 1.00 | 9.07 |
| ATOM | 1555 | CD1 | LEU | A | 227 | 78.440 | 52.388 | 14.548 | 1.00 | 10.12 |
| ATOM | 1556 | CD2 | LEU | A | 227 | 76.339 | 51.080 | 14.233 | 1.00 | 8.78 |
| ATOM | 1557 | C | LEU | A | 227 | 76.454 | 56.053 | 14.097 | 1.00 | 10.59 |
| ATOM | 1558 | O | LEU | A | 227 | 77.311 | 56.733 | 13.569 | 1.00 | 15.04 |
| ATOM | 1559 | N | ILE | A | 228 | 75.192 | 56.427 | 14.180 | 1.00 | 13.35 |
| ATOM | 1560 | CA | ILE | A | 228 | 74.689 | 57.682 | 13.630 | 1.00 | 14.10 |
| ATOM | 1561 | CB | ILE | A | 228 | 73.152 | 57.723 | 13.726 | 1.00 | 14.29 |
| ATOM | 1562 | CG2 | ILE | A | 228 | 72.568 | 58.876 | 12.919 | 1.00 | 16.27 |
| ATOM | 1563 | CG1 | ILE | A | 228 | 72.572 | 56.384 | 13.248 | 1.00 | 12.90 |
| ATOM | 1564 | CD1 | ILE | A | 228 | 71.062 | 56.299 | 13.299 | 1.00 | 10.99 |
| ATOM | 1565 | C | ILE | A | 228 | 75.347 | 58.910 | 14.301 | 1.00 | 20.58 |
| ATOM | 1566 | O | ILE | A | 228 | 75.481 | 59.994 | 13.676 | 1.00 | 18.93 |
| ATOM | 1567 | N | PHE | A | 229 | 75.805 | 58.728 | 15.568 | 1.00 | 21.36 |
| ATOM | 1568 | CA | PHE | A | 229 | 76.498 | 59.793 | 16.316 | 1.00 | 20.89 |
| ATOM | 1569 | CB | PHE | A | 229 | 76.331 | 59.595 | 17.837 | 1.00 | 19.68 |
| ATOM | 1570 | CG | PHE | A | 229 | 75.075 | 60.197 | 18.363 | 1.00 | 14.10 |
| ATOM | 1571 | CD1 | PHE | A | 229 | 73.934 | 59.446 | 18.471 | 1.00 | 13.94 |
| ATOM | 1572 | CD2 | PHE | A | 229 | 75.039 | 61.527 | 18.738 | 1.00 | 15.54 |
| ATOM | 1573 | CE1 | PHE | A | 229 | 72.763 | 60.003 | 18.949 | 1.00 | 14.85 |
| ATOM | 1574 | CE2 | PHE | A | 229 | 73.881 | 62.093 | 19.219 | 1.00 | 14.22 |
| ATOM | 1575 | CZ | PHE | A | 229 | 72.737 | 61.324 | 19.326 | 1.00 | 14.85 |
| ATOM | 1576 | C | PHE | A | 229 | 77.977 | 59.837 | 15.956 | 1.00 | 22.21 |
| ATOM | 1577 | O | PHE | A | 229 | 78.751 | 60.572 | 16.570 | 1.00 | 21.85 |
| ATOM | 1578 | N | GLY | A | 230 | 78.369 | 59.050 | 14.937 | 1.00 | 23.72 |
| ATOM | 1579 | CA | GLY | A | 230 | 79.756 | 59.023 | 14.484 | 1.00 | 22.28 |
| ATOM | 1580 | C | GLY | A | 230 | 80.677 | 58.185 | 15.357 | 1.00 | 21.94 |
| ATOM | 1581 | O | GLY | A | 230 | 81.896 | 58.282 | 15.256 | 1.00 | 20.64 |
| ATOM | 1582 | N | ALA | A | 231 | 80.104 | 57.378 | 16.226 | 1.00 | 23.66 |
| ATOM | 1583 | CA | ALA | A | 231 | 80.899 | 56.549 | 17.133 | 1.00 | 23.44 |
| ATOM | 1584 | CB | ALA | A | 231 | 79.975 | 55.750 | 18.052 | 1.00 | 24.45 |
| ATOM | 1585 | C | ALA | A | 231 | 81.813 | 55.597 | 16.381 | 1.00 | 22.95 |
| ATOM | 1586 | O | ALA | A | 231 | 81.337 | 54.811 | 15.554 | 1.00 | 23.77 |
| ATOM | 1587 | N | THR | A | 232 | 83.106 | 55.653 | 16.711 | 1.00 | 20.60 |
| ATOM | 1588 | CA | THR | A | 232 | 84.135 | 54.789 | 16.131 | 1.00 | 22.86 |
| ATOM | 1589 | CB | THR | A | 232 | 85.356 | 55.607 | 15.681 | 1.00 | 24.85 |
| ATOM | 1590 | OG1 | THR | A | 232 | 85.772 | 56.528 | 16.687 | 1.00 | 31.43 |
| ATOM | 1591 | CG2 | THR | A | 232 | 85.123 | 56.392 | 14.396 | 1.00 | 25.36 |
| ATOM | 1592 | C | THR | A | 232 | 84.555 | 53.706 | 17.126 | 1.00 | 21.66 |
| ATOM | 1593 | O | THR | A | 232 | 85.235 | 52.744 | 16.777 | 1.00 | 20.51 |
| ATOM | 1594 | N | ASP | A | 233 | 84.110 | 53.873 | 18.376 | 1.00 | 22.55 |
| ATOM | 1595 | CA | ASP | A | 233 | 84.381 | 52.930 | 19.470 | 1.00 | 19.98 |
| ATOM | 1596 | CB | ASP | A | 233 | 85.078 | 53.649 | 20.640 | 1.00 | 24.10 |
| ATOM | 1597 | CG | ASP | A | 233 | 85.181 | 52.837 | 21.952 | 1.00 | 26.33 |
| ATOM | 1598 | OD1 | ASP | A | 233 | 85.760 | 53.378 | 22.917 | 1.00 | 26.39 |
| ATOM | 1599 | OD2 | ASP | A | 233 | 84.685 | 51.687 | 22.016 | 1.00 | 22.93 |
| ATOM | 1600 | C | ASP | A | 233 | 83.059 | 52.339 | 19.886 | 1.00 | 17.40 |
| ATOM | 1601 | O | ASP | A | 233 | 82.259 | 52.976 | 20.577 | 1.00 | 19.65 |
| ATOM | 1602 | N | TYR | A | 234 | 82.803 | 51.144 | 19.399 | 1.00 | 15.12 |
| ATOM | 1603 | CA | TYR | A | 234 | 81.563 | 50.485 | 19.662 | 1.00 | 11.50 |
| ATOM | 1604 | CB | TYR | A | 234 | 80.512 | 50.868 | 18.619 | 1.00 | 19.52 |
| ATOM | 1605 | CG | TYR | A | 234 | 80.930 | 50.570 | 17.186 | 1.00 | 20.59 |
| ATOM | 1606 | CD1 | TYR | A | 234 | 80.771 | 49.299 | 16.656 | 1.00 | 22.28 |
| ATOM | 1607 | CE1 | TYR | A | 234 | 81.122 | 49.021 | 15.339 | 1.00 | 26.03 |
| ATOM | 1608 | CD2 | TYR | A | 234 | 81.458 | 51.566 | 16.374 | 1.00 | 20.66 |
| ATOM | 1609 | CE2 | TYR | A | 234 | 81.818 | 51.301 | 15.053 | 1.00 | 24.62 |
| ATOM | 1610 | CZ | TYR | A | 234 | 81.642 | 50.028 | 14.543 | 1.00 | 24.61 |
| ATOM | 1611 | OH | TYR | A | 234 | 81.992 | 49.752 | 13.244 | 1.00 | 26.95 |
| ATOM | 1612 | C | TYR | A | 234 | 81.754 | 48.998 | 19.778 | 1.00 | 7.69 |
| ATOM | 1613 | O | TYR | A | 234 | 82.839 | 48.483 | 19.622 | 1.00 | 11.49 |
| ATOM | 1614 | N | THR | A | 235 | 80.724 | 48.307 | 20.128 | 1.00 | 8.06 |
| ATOM | 1615 | CA | THR | A | 235 | 80.872 | 46.896 | 20.355 | 1.00 | 9.69 |
| ATOM | 1616 | CB | THR | A | 235 | 80.733 | 46.652 | 21.885 | 1.00 | 11.03 |
| ATOM | 1617 | OG1 | THR | A | 235 | 79.364 | 46.654 | 22.225 | 1.00 | 8.81 |
| ATOM | 1618 | CG2 | THR | A | 235 | 81.403 | 47.714 | 22.735 | 1.00 | 8.93 |
| ATOM | 1619 | C | THR | A | 235 | 79.809 | 46.101 | 19.661 | 1.00 | 11.93 |
| ATOM | 1620 | O | THR | A | 235 | 78.992 | 46.638 | 18.932 | 1.00 | 15.94 |
| ATOM | 1621 | N | SER | A | 236 | 79.846 | 44.807 | 19.942 | 1.00 | 16.37 |
| ATOM | 1622 | CA | SER | A | 236 | 78.943 | 43.800 | 19.422 | 1.00 | 16.47 |
| ATOM | 1623 | CB | SER | A | 236 | 79.388 | 42.436 | 19.914 | 1.00 | 17.79 |
| ATOM | 1624 | OG | SER | A | 236 | 80.708 | 42.187 | 19.457 | 1.00 | 23.99 |
| ATOM | 1625 | C | SER | A | 236 | 77.485 | 44.056 | 19.732 | 1.00 | 16.40 |
| ATOM | 1626 | O | SER | A | 236 | 76.613 | 43.391 | 19.145 | 1.00 | 15.77 |
| ATOM | 1627 | N | SER | A | 237 | 77.203 | 45.033 | 20.625 | 1.00 | 16.08 |
| ATOM | 1628 | CA | SER | A | 237 | 75.805 | 45.386 | 20.967 | 1.00 | 11.57 |
| ATOM | 1629 | CB | SER | A | 237 | 75.718 | 46.324 | 22.176 | 1.00 | 8.80 |
| ATOM | 1630 | OG | SER | A | 237 | 76.688 | 47.342 | 22.069 | 1.00 | 9.16 |
| ATOM | 1631 | C | SER | A | 237 | 75.123 | 46.065 | 19.768 | 1.00 | 10.30 |
| ATOM | 1632 | O | SER | A | 237 | 73.898 | 46.129 | 19.703 | 1.00 | 7.31 |
| ATOM | 1633 | N | ILE | A | 238 | 75.914 | 46.546 | 18.803 | 1.00 | 11.34 |
| ATOM | 1634 | CA | ILE | A | 238 | 75.259 | 47.142 | 17.602 | 1.00 | 14.03 |
| ATOM | 1635 | CB | ILE | A | 238 | 76.223 | 47.862 | 16.625 | 1.00 | 14.92 |
| ATOM | 1636 | CG2 | ILE | A | 238 | 76.952 | 49.028 | 17.293 | 1.00 | 14.36 |
| ATOM | 1637 | CG1 | ILE | A | 238 | 77.197 | 46.891 | 15.967 | 1.00 | 13.71 |
| ATOM | 1638 | CD1 | ILE | A | 238 | 77.847 | 47.457 | 14.706 | 1.00 | 15.09 |
| ATOM | 1639 | C | ILE | A | 238 | 74.478 | 46.039 | 16.869 | 1.00 | 10.92 |
| ATOM | 1640 | O | ILE | A | 238 | 73.360 | 46.251 | 16.437 | 1.00 | 14.44 |
| ATOM | 1641 | N | ASP | A | 239 | 75.053 | 44.828 | 16.796 | 1.00 | 11.90 |
| ATOM | 1642 | CA | ASP | A | 239 | 74.359 | 43.686 | 16.165 | 1.00 | 12.25 |
| ATOM | 1643 | CB | ASP | A | 239 | 75.237 | 42.412 | 16.174 | 1.00 | 10.53 |
| ATOM | 1644 | CG | ASP | A | 239 | 76.350 | 42.470 | 15.144 | 1.00 | 12.86 |
| ATOM | 1645 | OD1 | ASP | A | 239 | 76.333 | 43.412 | 14.319 | 1.00 | 13.57 |
| ATOM | 1646 | OD2 | ASP | A | 239 | 77.244 | 41.584 | 15.165 | 1.00 | 13.48 |
| ATOM | 1647 | C | ASP | A | 239 | 73.047 | 43.422 | 16.859 | 1.00 | 14.25 |
| ATOM | 1648 | O | ASP | A | 239 | 72.034 | 43.071 | 16.204 | 1.00 | 14.57 |
| ATOM | 1649 | N | VAL | A | 240 | 73.045 | 43.626 | 18.220 | 1.00 | 13.55 |
| ATOM | 1650 | CA | VAL | A | 240 | 71.833 | 43.435 | 19.024 | 1.00 | 7.80 |
| ATOM | 1651 | CB | VAL | A | 240 | 72.101 | 43.523 | 20.575 | 1.00 | 9.66 |
| ATOM | 1652 | CG1 | VAL | A | 240 | 70.786 | 43.446 | 21.328 | 1.00 | 5.00 |
| ATOM | 1653 | CG2 | VAL | A | 240 | 73.036 | 42.420 | 21.023 | 1.00 | 9.17 |
| ATOM | 1654 | C | VAL | A | 240 | 70.749 | 44.429 | 18.640 | 1.00 | 5.00 |
| ATOM | 1655 | O | VAL | A | 240 | 69.593 | 44.071 | 18.522 | 1.00 | 9.41 |
| ATOM | 1656 | N | TRP | A | 241 | 71.114 | 45.677 | 18.441 | 1.00 | 8.68 |
| ATOM | 1657 | CA | TRP | A | 241 | 70.115 | 46.682 | 18.039 | 1.00 | 8.70 |
| ATOM | 1658 | CB | TRP | A | 241 | 70.785 | 48.063 | 17.942 | 1.00 | 12.59 |
| ATOM | 1659 | CG | TRP | A | 241 | 69.907 | 49.159 | 17.376 | 1.00 | 13.08 |
| ATOM | 1660 | CD2 | TRP | A | 241 | 69.086 | 50.093 | 18.105 | 1.00 | 17.91 |
| ATOM | 1661 | CE2 | TRP | A | 241 | 68.435 | 50.922 | 17.145 | 1.00 | 16.74 |
| ATOM | 1662 | CE3 | TRP | A | 241 | 68.824 | 50.309 | 19.466 | 1.00 | 15.70 |
| ATOM | 1663 | CD1 | TRP | A | 241 | 69.738 | 49.474 | 16.053 | 1.00 | 16.26 |
| ATOM | 1664 | NE1 | TRP | A | 241 | 68.841 | 50.515 | 15.916 | 1.00 | 15.52 |
| ATOM | 1665 | CZ2 | TRP | A | 241 | 67.552 | 51.940 | 17.507 | 1.00 | 19.78 |
| ATOM | 1666 | CZ3 | TRP | A | 241 | 67.952 | 51.317 | 19.820 | 1.00 | 17.55 |
| ATOM | 1667 | CH2 | TRP | A | 241 | 67.323 | 52.123 | 18.847 | 1.00 | 19.91 |
| ATOM | 1668 | C | TRP | A | 241 | 69.501 | 46.257 | 16.672 | 1.00 | 9.05 |
| ATOM | 1669 | O | TRP | A | 241 | 68.294 | 46.321 | 16.466 | 1.00 | 8.77 |
| ATOM | 1670 | N | SER | A | 242 | 70.357 | 45.787 | 15.756 | 1.00 | 9.89 |
| ATOM | 1671 | CA | SER | A | 242 | 69.900 | 45.326 | 14.419 | 1.00 | 11.70 |
| ATOM | 1672 | CB | SER | A | 242 | 71.097 | 44.938 | 13.521 | 1.00 | 5.00 |
| ATOM | 1673 | OG | SER | A | 242 | 71.927 | 46.063 | 13.328 | 1.00 | 7.55 |
| ATOM | 1674 | C | SER | A | 242 | 68.926 | 44.177 | 14.578 | 1.00 | 11.06 |
| ATOM | 1675 | O | SER | A | 242 | 67.824 | 44.224 | 14.029 | 1.00 | 13.94 |
| ATOM | 1676 | N | ALA | A | 243 | 69.305 | 43.169 | 15.398 | 1.00 | 13.36 |
| ATOM | 1677 | CA | ALA | A | 243 | 68.402 | 42.036 | 15.672 | 1.00 | 14.87 |
| ATOM | 1678 | CB | ALA | A | 243 | 69.036 | 41.047 | 16.635 | 1.00 | 16.56 |
| ATOM | 1679 | C | ALA | A | 243 | 67.075 | 42.532 | 16.211 | 1.00 | 15.72 |
| ATOM | 1680 | O | ALA | A | 243 | 66.005 | 42.050 | 15.816 | 1.00 | 17.54 |
| ATOM | 1681 | N | GLY | A | 244 | 67.130 | 43.540 | 17.093 | 1.00 | 17.52 |
| ATOM | 1682 | CA | GLY | A | 244 | 65.897 | 44.097 | 17.620 | 1.00 | 16.24 |
| ATOM | 1683 | C | GLY | A | 244 | 65.090 | 44.768 | 16.518 | 1.00 | 16.45 |
| ATOM | 1684 | O | GLY | A | 244 | 63.877 | 44.692 | 16.493 | 1.00 | 14.27 |
| ATOM | 1685 | N | CYS | A | 245 | 65.786 | 45.411 | 15.585 | 1.00 | 18.79 |
| ATOM | 1686 | CA | CYS | A | 245 | 65.118 | 46.062 | 14.447 | 1.00 | 19.45 |
| ATOM | 1687 | CB | CYS | A | 245 | 66.139 | 46.841 | 13.614 | 1.00 | 16.90 |
| ATOM | 1688 | SG | CYS | A | 245 | 66.564 | 48.451 | 14.319 | 1.00 | 17.99 |
| ATOM | 1689 | C | CYS | A | 245 | 64.407 | 44.993 | 13.608 | 1.00 | 17.15 |
| ATOM | 1690 | O | CYS | A | 245 | 63.258 | 45.147 | 13.238 | 1.00 | 18.70 |
| ATOM | 1691 | N | VAL | A | 246 | 65.078 | 43.872 | 13.389 | 1.00 | 17.86 |
| ATOM | 1692 | CA | VAL | A | 246 | 64.478 | 42.754 | 12.668 | 1.00 | 19.17 |
| ATOM | 1693 | CB | VAL | A | 246 | 65.465 | 41.586 | 12.519 | 1.00 | 16.94 |
| ATOM | 1694 | CG1 | VAL | A | 246 | 64.803 | 40.416 | 11.810 | 1.00 | 16.17 |
| ATOM | 1695 | CG2 | VAL | A | 246 | 66.713 | 42.023 | 11.797 | 1.00 | 20.27 |
| ATOM | 1696 | C | VAL | A | 246 | 63.232 | 42.244 | 13.386 | 1.00 | 21.86 |
| ATOM | 1697 | O | VAL | A | 246 | 62.177 | 42.028 | 12.758 | 1.00 | 22.61 |
| ATOM | 1698 | N | LEU | A | 247 | 63.336 | 42.044 | 14.726 | 1.00 | 22.24 |
| ATOM | 1699 | CA | LEU | A | 247 | 62.189 | 41.556 | 15.520 | 1.00 | 17.75 |
| ATOM | 1700 | CB | LEU | A | 247 | 62.556 | 41.563 | 17.018 | 1.00 | 21.43 |
| ATOM | 1701 | CG | LEU | A | 247 | 61.857 | 40.556 | 17.931 | 1.00 | 21.52 |
| ATOM | 1702 | CD1 | LEU | A | 247 | 61.533 | 41.174 | 19.293 | 1.00 | 21.62 |
| ATOM | 1703 | CD2 | LEU | A | 247 | 60.624 | 39.960 | 17.283 | 1.00 | 19.10 |
| ATOM | 1704 | C | LEU | A | 247 | 60.984 | 42.438 | 15.331 | 1.00 | 16.58 |
| ATOM | 1705 | O | LEU | A | 247 | 59.882 | 41.963 | 15.074 | 1.00 | 17.00 |
| ATOM | 1706 | N | ALA | A | 248 | 61.190 | 43.732 | 15.508 | 1.00 | 17.51 |
| ATOM | 1707 | CA | ALA | A | 248 | 60.117 | 44.718 | 15.399 | 1.00 | 17.76 |
| ATOM | 1708 | CB | ALA | A | 248 | 60.675 | 46.096 | 15.718 | 1.00 | 13.92 |
| ATOM | 1709 | C | ALA | A | 248 | 59.479 | 44.694 | 14.001 | 1.00 | 19.04 |
| ATOM | 1710 | O | ALA | A | 248 | 58.248 | 44.674 | 13.868 | 1.00 | 19.92 |
| ATOM | 1711 | N | GLU | A | 249 | 60.326 | 44.675 | 12.956 | 1.00 | 19.92 |
| ATOM | 1712 | CA | GLU | A | 249 | 59.836 | 44.623 | 11.570 | 1.00 | 18.88 |
| ATOM | 1713 | CB | GLU | A | 249 | 61.017 | 44.571 | 10.588 | 1.00 | 22.05 |
| ATOM | 1714 | CG | GLU | A | 249 | 60.629 | 44.870 | 9.143 | 1.00 | 24.95 |
| ATOM | 1715 | CD | GLU | A | 249 | 61.815 | 44.888 | 8.200 | 1.00 | 26.40 |
| ATOM | 1716 | OE1 | GLU | A | 249 | 62.860 | 45.461 | 8.573 | 1.00 | 27.15 |
| ATOM | 1717 | OE2 | GLU | A | 249 | 61.699 | 44.330 | 7.086 | 1.00 | 26.13 |
| ATOM | 1718 | C | GLU | A | 249 | 58.942 | 43.404 | 11.383 | 1.00 | 16.29 |
| ATOM | 1719 | O | GLU | A | 249 | 57.857 | 43.488 | 10.842 | 1.00 | 16.64 |
| ATOM | 1720 | N | LEU | A | 250 | 59.391 | 42.265 | 11.872 | 1.00 | 18.94 |
| ATOM | 1721 | CA | LEU | A | 250 | 58.611 | 41.041 | 11.765 | 1.00 | 19.63 |
| ATOM | 1722 | CB | LEU | A | 250 | 59.421 | 39.855 | 12.227 | 1.00 | 21.77 |
| ATOM | 1723 | CG | LEU | A | 250 | 60.508 | 39.410 | 11.255 | 1.00 | 21.90 |
| ATOM | 1724 | CD1 | LEU | A | 250 | 61.205 | 38.171 | 11.789 | 1.00 | 22.15 |
| ATOM | 1725 | CD2 | LEU | A | 250 | 59.894 | 39.138 | 9.882 | 1.00 | 20.69 |
| ATOM | 1726 | C | LEU | A | 250 | 57.257 | 41.102 | 12.453 | 1.00 | 21.42 |
| ATOM | 1727 | O | LEU | A | 250 | 56.302 | 40.428 | 12.029 | 1.00 | 19.97 |
| ATOM | 1728 | N | LEU | A | 251 | 57.153 | 41.914 | 13.513 | 1.00 | 22.22 |
| ATOM | 1729 | CA | LEU | A | 251 | 55.886 | 42.050 | 14.226 | 1.00 | 22.60 |
| ATOM | 1730 | CB | LEU | A | 251 | 56.111 | 42.386 | 15.712 | 1.00 | 21.94 |
| ATOM | 1731 | CG | LEU | A | 251 | 57.047 | 41.470 | 16.505 | 1.00 | 23.22 |
| ATOM | 1732 | CD1 | LEU | A | 251 | 57.607 | 42.223 | 17.699 | 1.00 | 22.21 |
| ATOM | 1733 | CD2 | LEU | A | 251 | 56.323 | 40.209 | 16.955 | 1.00 | 20.09 |
| ATOM | 1734 | C | LEU | A | 251 | 55.039 | 43.150 | 13.598 | 1.00 | 22.16 |
| ATOM | 1735 | O | LEU | A | 251 | 53.833 | 43.063 | 13.578 | 1.00 | 21.07 |
| ATOM | 1736 | N | LEU | A | 252 | 55.699 | 44.193 | 13.109 | 1.00 | 24.69 |
| ATOM | 1737 | CA | LEU | A | 252 | 55.026 | 45.333 | 12.497 | 1.00 | 27.50 |
| ATOM | 1738 | CB | LEU | A | 252 | 55.990 | 46.519 | 12.422 | 1.00 | 28.83 |
| ATOM | 1739 | CG | LEU | A | 252 | 55.884 | 47.597 | 13.506 | 1.00 | 31.65 |
| ATOM | 1740 | CD1 | LEU | A | 252 | 54.636 | 47.423 | 14.368 | 1.00 | 29.59 |
| ATOM | 1741 | CD2 | LEU | A | 252 | 57.143 | 47.620 | 14.358 | 1.00 | 30.57 |
| ATOM | 1742 | C | LEU | A | 252 | 54.532 | 45.033 | 11.082 | 1.00 | 28.49 |
| ATOM | 1743 | O | LEU | A | 252 | 53.475 | 45.518 | 10.676 | 1.00 | 29.11 |
| ATOM | 1744 | N | GLY | A | 253 | 55.328 | 44.283 | 10.320 | 1.00 | 29.33 |
| ATOM | 1745 | CA | GLY | A | 253 | 54.978 | 43.997 | 8.933 | 1.00 | 29.31 |
| ATOM | 1746 | C | GLY | A | 253 | 55.609 | 45.024 | 8.000 | 1.00 | 30.52 |
| ATOM | 1747 | O | GLY | A | 253 | 55.243 | 45.121 | 6.829 | 1.00 | 31.13 |
| ATOM | 1748 | N | GLN | A | 254 | 56.567 | 45.795 | 8.548 | 1.00 | 31.15 |
| ATOM | 1749 | CA | GLN | A | 254 | 57.308 | 46.846 | 7.841 | 1.00 | 30.85 |
| ATOM | 1750 | CB | GLN | A | 254 | 56.375 | 47.991 | 7.462 | 1.00 | 31.46 |
| ATOM | 1751 | CG | GLN | A | 254 | 55.850 | 48.778 | 8.651 | 1.00 | 30.44 |
| ATOM | 1752 | CD | GLN | A | 254 | 54.831 | 49.815 | 8.251 | 1.00 | 29.54 |
| ATOM | 1753 | OE1 | GLN | A | 254 | 55.153 | 50.984 | 8.096 | 1.00 | 30.99 |
| ATOM | 1754 | NE2 | GLN | A | 254 | 53.591 | 49.391 | 8.089 | 1.00 | 32.26 |
| ATOM | 1755 | C | GLN | A | 254 | 58.416 | 47.378 | 8.747 | 1.00 | 30.06 |
| ATOM | 1756 | O | GLN | A | 254 | 58.281 | 47.323 | 9.967 | 1.00 | 31.81 |
| ATOM | 1757 | N | PRO | A | 255 | 59.525 | 47.896 | 8.182 | 1.00 | 28.75 |
| ATOM | 1758 | CD | PRO | A | 255 | 59.786 | 48.003 | 6.736 | 1.00 | 29.77 |
| ATOM | 1759 | CA | PRO | A | 255 | 60.639 | 48.422 | 8.978 | 1.00 | 27.17 |
| ATOM | 1760 | CB | PRO | A | 255 | 61.503 | 49.146 | 7.955 | 1.00 | 28.27 |
| ATOM | 1761 | CG | PRO | A | 255 | 61.229 | 48.421 | 6.674 | 1.00 | 30.04 |
| ATOM | 1762 | C | PRO | A | 255 | 60.166 | 49.386 | 10.059 | 1.00 | 25.98 |
| ATOM | 1763 | O | PRO | A | 255 | 59.328 | 50.262 | 9.814 | 1.00 | 24.39 |
| ATOM | 1764 | N | ILE | A | 256 | 60.699 | 49.214 | 11.265 | 1.00 | 23.84 |
| ATOM | 1765 | CA | ILE | A | 256 | 60.301 | 50.071 | 12.381 | 1.00 | 20.82 |
| ATOM | 1766 | CB | ILE | A | 256 | 60.640 | 49.441 | 13.753 | 1.00 | 18.45 |
| ATOM | 1767 | CG2 | ILE | A | 256 | 62.134 | 49.177 | 13.867 | 1.00 | 14.74 |
| ATOM | 1768 | CG1 | ILE | A | 256 | 60.145 | 50.352 | 14.887 | 1.00 | 20.07 |
| ATOM | 1769 | CD1 | ILE | A | 256 | 59.894 | 49.626 | 16.196 | 1.00 | 22.63 |
| ATOM | 1770 | C | ILE | A | 256 | 60.849 | 51.496 | 12.267 | 1.00 | 18.31 |
| ATOM | 1771 | O | ILE | A | 256 | 60.143 | 52.459 | 12.578 | 1.00 | 19.96 |
| ATOM | 1772 | N | PHE | A | 257 | 62.092 | 51.623 | 11.832 | 1.00 | 17.97 |
| ATOM | 1773 | CA | PHE | A | 257 | 62.735 | 52.925 | 11.683 | 1.00 | 19.27 |
| ATOM | 1774 | CB | PHE | A | 257 | 63.953 | 53.041 | 12.641 | 1.00 | 20.54 |
| ATOM | 1775 | CG | PHE | A | 257 | 63.683 | 52.674 | 14.091 | 1.00 | 18.05 |
| ATOM | 1776 | CD1 | PHE | A | 257 | 62.675 | 53.297 | 14.806 | 1.00 | 18.44 |
| ATOM | 1777 | CD2 | PHE | A | 257 | 64.462 | 51.729 | 14.740 | 1.00 | 19.25 |
| ATOM | 1778 | CE1 | PHE | A | 257 | 62.449 | 52.982 | 16.145 | 1.00 | 19.85 |
| ATOM | 1779 | CE2 | PHE | A | 257 | 64.235 | 51.405 | 16.083 | 1.00 | 19.59 |
| ATOM | 1780 | CZ | PHE | A | 257 | 63.229 | 52.038 | 16.779 | 1.00 | 14.97 |
| ATOM | 1781 | C | PHE | A | 257 | 63.194 | 53.151 | 10.217 | 1.00 | 19.61 |
| ATOM | 1782 | O | PHE | A | 257 | 64.343 | 52.926 | 9.880 | 1.00 | 19.87 |
| ATOM | 1783 | N | PRO | A | 258 | 62.297 | 53.588 | 9.329 | 1.00 | 21.18 |
| ATOM | 1784 | CD | PRO | A | 258 | 60.880 | 53.883 | 9.604 | 1.00 | 22.67 |
| ATOM | 1785 | CA | PRO | A | 258 | 62.632 | 53.821 | 7.908 | 1.00 | 24.51 |
| ATOM | 1786 | CB | PRO | A | 258 | 61.266 | 53.704 | 7.242 | 1.00 | 22.28 |
| ATOM | 1787 | CG | PRO | A | 258 | 60.350 | 54.313 | 8.249 | 1.00 | 25.70 |
| ATOM | 1788 | C | PRO | A | 258 | 63.204 | 55.207 | 7.656 | 1.00 | 23.60 |
| ATOM | 1789 | O | PRO | A | 258 | 62.481 | 56.116 | 7.287 | 1.00 | 25.61 |
| ATOM | 1790 | N | GLY | A | 259 | 64.496 | 55.361 | 7.842 | 1.00 | 26.01 |
| ATOM | 1791 | CA | GLY | A | 259 | 65.120 | 56.657 | 7.608 | 1.00 | 29.65 |
| ATOM | 1792 | C | GLY | A | 259 | 65.385 | 56.916 | 6.121 | 1.00 | 31.04 |
| ATOM | 1793 | O | GLY | A | 259 | 65.717 | 55.985 | 5.389 | 1.00 | 30.01 |
| ATOM | 1794 | N | ASP | A | 260 | 65.232 | 58.181 | 5.684 | 1.00 | 31.88 |
| ATOM | 1795 | CA | ASP | A | 260 | 65.450 | 58.560 | 4.282 | 1.00 | 33.04 |
| ATOM | 1796 | CB | ASP | A | 260 | 64.452 | 59.635 | 3.814 | 1.00 | 33.94 |
| ATOM | 1797 | CG | ASP | A | 260 | 64.645 | 60.997 | 4.466 | 1.00 | 35.58 |
| ATOM | 1798 | OD1 | ASP | A | 260 | 65.719 | 61.250 | 5.023 | 1.00 | 35.78 |
| ATOM | 1799 | OD2 | ASP | A | 260 | 63.713 | 61.819 | 4.407 | 1.00 | 38.52 |
| ATOM | 1800 | C | ASP | A | 260 | 66.897 | 58.962 | 4.008 | 1.00 | 32.63 |
| ATOM | 1801 | O | ASP | A | 260 | 67.258 | 59.313 | 2.875 | 1.00 | 33.30 |
| ATOM | 1802 | N | SER | A | 261 | 67.709 | 58.884 | 5.056 | 1.00 | 31.36 |
| ATOM | 1803 | CA | SER | A | 261 | 69.145 | 59.189 | 5.043 | 1.00 | 30.25 |
| ATOM | 1804 | CB | SER | A | 261 | 69.435 | 60.692 | 4.935 | 1.00 | 32.71 |
| ATOM | 1805 | OG | SER | A | 261 | 69.062 | 61.392 | 6.117 | 1.00 | 33.13 |
| ATOM | 1806 | C | SER | A | 261 | 69.769 | 58.623 | 6.308 | 1.00 | 28.76 |
| ATOM | 1807 | O | SER | A | 261 | 69.058 | 58.090 | 7.146 | 1.00 | 28.26 |
| ATOM | 1808 | N | GLY | A | 262 | 71.092 | 58.725 | 6.423 | 1.00 | 28.70 |
| ATOM | 1809 | CA | GLY | A | 262 | 71.808 | 58.208 | 7.576 | 1.00 | 25.78 |
| ATOM | 1810 | C | GLY | A | 262 | 71.576 | 59.021 | 8.851 | 1.00 | 26.60 |
| ATOM | 1811 | O | GLY | A | 262 | 71.723 | 58.491 | 9.959 | 1.00 | 23.39 |
| ATOM | 1812 | N | VAL | A | 263 | 71.209 | 60.304 | 8.681 | 1.00 | 25.15 |
| ATOM | 1813 | CA | VAL | A | 263 | 70.944 | 61.211 | 9.799 | 1.00 | 23.70 |
| ATOM | 1814 | CB | VAL | A | 263 | 71.429 | 62.643 | 9.489 | 1.00 | 24.57 |
| ATOM | 1815 | CG1 | VAL | A | 263 | 70.777 | 63.679 | 10.400 | 1.00 | 26.49 |
| ATOM | 1816 | CG2 | VAL | A | 263 | 72.940 | 62.719 | 9.585 | 1.00 | 24.46 |
| ATOM | 1817 | C | VAL | A | 263 | 69.463 | 61.213 | 10.126 | 1.00 | 23.60 |
| ATOM | 1818 | O | VAL | A | 263 | 69.074 | 61.339 | 11.298 | 1.00 | 23.21 |
| ATOM | 1819 | N | ASP | A | 264 | 68.635 | 61.037 | 9.086 | 1.00 | 18.63 |
| ATOM | 1820 | CA | ASP | A | 264 | 67.189 | 60.977 | 9.243 | 1.00 | 16.12 |
| ATOM | 1821 | CB | ASP | A | 264 | 66.516 | 61.007 | 7.873 | 1.00 | 16.93 |
| ATOM | 1822 | CG | ASP | A | 264 | 65.023 | 61.085 | 7.967 | 1.00 | 15.99 |
| ATOM | 1823 | OD1 | ASP | A | 264 | 64.518 | 62.131 | 8.361 | 1.00 | 23.33 |
| ATOM | 1824 | OD2 | ASP | A | 264 | 64.353 | 60.101 | 7.645 | 1.00 | 21.38 |
| ATOM | 1825 | C | ASP | A | 264 | 66.799 | 59.688 | 9.956 | 1.00 | 14.34 |
| ATOM | 1826 | O | ASP | A | 264 | 65.708 | 59.566 | 10.512 | 1.00 | 11.71 |
| ATOM | 1827 | N | GLN | A | 265 | 67.712 | 58.739 | 9.919 | 1.00 | 13.50 |
| ATOM | 1828 | CA | GLN | A | 265 | 67.543 | 57.453 | 10.551 | 1.00 | 16.78 |
| ATOM | 1829 | CB | GLN | A | 265 | 68.761 | 56.593 | 10.222 | 1.00 | 15.08 |
| ATOM | 1830 | CG | GLN | A | 265 | 68.718 | 55.172 | 10.751 | 1.00 | 22.06 |
| ATOM | 1831 | CD | GLN | A | 265 | 67.534 | 54.379 | 10.241 | 1.00 | 24.38 |
| ATOM | 1832 | OE1 | GLN | A | 265 | 66.925 | 54.718 | 9.216 | 1.00 | 26.93 |
| ATOM | 1833 | NE2 | GLN | A | 265 | 67.195 | 53.317 | 10.957 | 1.00 | 25.58 |
| ATOM | 1834 | C | GLN | A | 265 | 67.420 | 57.667 | 12.082 | 1.00 | 18.49 |
| ATOM | 1835 | O | GLN | A | 265 | 66.649 | 56.981 | 12.750 | 1.00 | 17.65 |
| ATOM | 1836 | N | LEU | A | 266 | 68.158 | 58.660 | 12.595 | 1.00 | 20.41 |
| ATOM | 1837 | CA | LEU | A | 266 | 68.120 | 59.024 | 14.015 | 1.00 | 23.21 |
| ATOM | 1838 | CB | LEU | A | 266 | 69.293 | 59.962 | 14.385 | 1.00 | 19.14 |
| ATOM | 1839 | CG | LEU | A | 266 | 69.499 | 60.258 | 15.898 | 1.00 | 22.99 |
| ATOM | 1840 | CD1 | LEU | A | 266 | 69.548 | 58.966 | 16.726 | 1.00 | 13.89 |
| ATOM | 1841 | CD2 | LEU | A | 266 | 70.754 | 61.085 | 16.119 | 1.00 | 19.84 |
| ATOM | 1842 | C | LEU | A | 266 | 66.774 | 59.677 | 14.342 | 1.00 | 24.24 |
| ATOM | 1843 | O | LEU | A | 266 | 66.171 | 59.387 | 15.394 | 1.00 | 25.95 |
| ATOM | 1844 | N | VAL | A | 267 | 66.284 | 60.541 | 13.428 | 1.00 | 20.54 |
| ATOM | 1845 | CA | VAL | A | 267 | 64.997 | 61.209 | 13.623 | 1.00 | 21.02 |
| ATOM | 1846 | CB | VAL | A | 267 | 64.661 | 62.250 | 12.512 | 1.00 | 22.65 |
| ATOM | 1847 | CG1 | VAL | A | 267 | 63.242 | 62.786 | 12.679 | 1.00 | 20.94 |
| ATOM | 1848 | CG2 | VAL | A | 267 | 65.659 | 63.397 | 12.492 | 1.00 | 23.85 |
| ATOM | 1849 | C | VAL | A | 267 | 63.867 | 60.202 | 13.712 | 1.00 | 21.30 |
| ATOM | 1850 | O | VAL | A | 267 | 62.916 | 60.389 | 14.487 | 1.00 | 21.41 |
| ATOM | 1851 | N | GLU | A | 268 | 63.961 | 59.136 | 12.904 | 1.00 | 21.14 |
| ATOM | 1852 | CA | GLU | A | 268 | 62.940 | 58.087 | 12.880 | 1.00 | 21.32 |
| ATOM | 1853 | CB | GLU | A | 268 | 63.188 | 57.109 | 11.714 | 1.00 | 20.56 |
| ATOM | 1854 | CG | GLU | A | 268 | 62.780 | 57.634 | 10.337 | 1.00 | 19.52 |
| ATOM | 1855 | CD | GLU | A | 268 | 61.323 | 58.007 | 10.270 | 1.00 | 15.74 |
| ATOM | 1856 | OE1 | GLU | A | 268 | 61.022 | 59.170 | 9.958 | 1.00 | 19.10 |
| ATOM | 1857 | OE2 | GLU | A | 268 | 60.483 | 57.137 | 10.542 | 1.00 | 21.33 |
| ATOM | 1858 | C | GLU | A | 268 | 62.947 | 57.315 | 14.203 | 1.00 | 20.77 |
| ATOM | 1859 | O | GLU | A | 268 | 61.900 | 56.973 | 14.744 | 1.00 | 21.32 |
| ATOM | 1860 | N | ILE | A | 269 | 64.133 | 57.042 | 14.704 | 1.00 | 20.21 |
| ATOM | 1861 | CA | ILE | A | 269 | 64.272 | 56.315 | 15.965 | 1.00 | 22.93 |
| ATOM | 1862 | CB | ILE | A | 269 | 65.753 | 56.013 | 16.272 | 1.00 | 21.57 |
| ATOM | 1863 | CG2 | ILE | A | 269 | 65.916 | 55.490 | 17.704 | 1.00 | 21.70 |
| ATOM | 1864 | CG1 | ILE | A | 269 | 66.320 | 55.012 | 15.261 | 1.00 | 16.65 |
| ATOM | 1865 | CD1 | ILE | A | 269 | 67.827 | 54.977 | 15.231 | 1.00 | 12.77 |
| ATOM | 1866 | C | ILE | A | 269 | 63.680 | 57.149 | 17.106 | 1.00 | 22.44 |
| ATOM | 1867 | O | ILE | A | 269 | 62.822 | 56.684 | 17.835 | 1.00 | 22.07 |
| ATOM | 1868 | N | ILE | A | 270 | 64.150 | 58.393 | 17.208 | 1.00 | 24.28 |
| ATOM | 1869 | CA | ILE | A | 270 | 63.711 | 59.347 | 18.209 | 1.00 | 26.56 |
| ATOM | 1870 | CB | ILE | A | 270 | 64.358 | 60.727 | 17.967 | 1.00 | 24.36 |
| ATOM | 1871 | CG2 | ILE | A | 270 | 63.698 | 61.791 | 18.831 | 1.00 | 27.65 |
| ATOM | 1872 | CG1 | ILE | A | 270 | 65.853 | 60.652 | 18.264 | 1.00 | 20.73 |
| ATOM | 1873 | CD1 | ILE | A | 270 | 66.567 | 61.983 | 18.215 | 1.00 | 18.62 |
| ATOM | 1874 | C | ILE | A | 270 | 62.195 | 59.480 | 18.234 | 1.00 | 29.28 |
| ATOM | 1875 | O | ILE | A | 270 | 61.588 | 59.653 | 19.301 | 1.00 | 28.79 |
| ATOM | 1876 | N | LYS | A | 271 | 61.591 | 59.401 | 17.054 | 1.00 | 29.05 |
| ATOM | 1877 | CA | LYS | A | 271 | 60.144 | 59.519 | 16.903 | 1.00 | 28.68 |
| ATOM | 1878 | CB | LYS | A | 271 | 59.815 | 59.543 | 15.402 | 1.00 | 32.12 |
| ATOM | 1879 | CG | LYS | A | 271 | 58.345 | 59.527 | 15.052 | 1.00 | 35.46 |
| ATOM | 1880 | CD | LYS | A | 271 | 58.159 | 59.612 | 13.536 | 1.00 | 39.81 |
| ATOM | 1881 | CE | LYS | A | 271 | 58.964 | 60.767 | 12.942 | 1.00 | 39.81 |
| ATOM | 1882 | NZ | LYS | A | 271 | 58.903 | 60.792 | 11.455 | 1.00 | 42.86 |
| ATOM | 1883 | C | LYS | A | 271 | 59.414 | 58.393 | 17.625 | 1.00 | 28.22 |
| ATOM | 1884 | O | LYS | A | 271 | 58.219 | 58.488 | 17.897 | 1.00 | 26.93 |
| ATOM | 1885 | N | VAL | A | 272 | 60.144 | 57.334 | 17.950 | 1.00 | 28.27 |
| ATOM | 1886 | CA | VAL | A | 272 | 59.578 | 56.195 | 18.655 | 1.00 | 27.38 |
| ATOM | 1887 | CB | VAL | A | 272 | 59.837 | 54.878 | 17.904 | 1.00 | 27.95 |
| ATOM | 1888 | CG1 | VAL | A | 272 | 59.334 | 53.688 | 18.710 | 1.00 | 27.27 |
| ATOM | 1889 | CG2 | VAL | A | 272 | 59.184 | 54.908 | 16.531 | 1.00 | 26.96 |
| ATOM | 1890 | C | VAL | A | 272 | 60.123 | 56.080 | 20.091 | 1.00 | 27.25 |
| ATOM | 1891 | O | VAL | A | 272 | 59.361 | 55.807 | 21.012 | 1.00 | 28.50 |
| ATOM | 1892 | N | LEU | A | 273 | 61.439 | 56.266 | 20.263 | 1.00 | 25.88 |
| ATOM | 1893 | CA | LEU | A | 273 | 62.096 | 56.160 | 21.573 | 1.00 | 25.89 |
| ATOM | 1894 | CB | LEU | A | 273 | 63.515 | 55.612 | 21.394 | 1.00 | 22.17 |
| ATOM | 1895 | CG | LEU | A | 273 | 63.671 | 54.117 | 21.116 | 1.00 | 21.67 |
| ATOM | 1896 | CD1 | LEU | A | 273 | 65.066 | 53.656 | 21.519 | 1.00 | 23.34 |
| ATOM | 1897 | CD2 | LEU | A | 273 | 62.614 | 53.308 | 21.842 | 1.00 | 22.69 |
| ATOM | 1898 | C | LEU | A | 273 | 62.198 | 57.510 | 22.304 | 1.00 | 27.83 |
| ATOM | 1899 | O | LEU | A | 273 | 62.869 | 57.608 | 23.342 | 1.00 | 30.31 |
| ATOM | 1900 | N | GLY | A | 274 | 61.577 | 58.545 | 21.746 | 1.00 | 26.13 |
| ATOM | 1901 | CA | GLY | A | 274 | 61.641 | 59.866 | 22.327 | 1.00 | 23.58 |
| ATOM | 1902 | C | GLY | A | 274 | 63.038 | 60.415 | 22.272 | 1.00 | 23.20 |
| ATOM | 1903 | O | GLY | A | 274 | 63.972 | 59.679 | 22.001 | 1.00 | 24.94 |
| ATOM | 1904 | N | THR | A | 275 | 63.194 | 61.706 | 22.523 | 1.00 | 23.62 |
| ATOM | 1905 | CA | THR | A | 275 | 64.515 | 62.318 | 22.484 | 1.00 | 25.71 |
| ATOM | 1906 | CB | THR | A | 275 | 64.440 | 63.805 | 22.849 | 1.00 | 24.96 |
| ATOM | 1907 | OG1 | THR | A | 275 | 63.379 | 64.466 | 22.191 | 1.00 | 27.34 |
| ATOM | 1908 | CG2 | THR | A | 275 | 65.714 | 64.569 | 22.589 | 1.00 | 24.20 |
| ATOM | 1909 | C | THR | A | 275 | 65.469 | 61.619 | 23.445 | 1.00 | 27.31 |
| ATOM | 1910 | O | THR | A | 275 | 65.094 | 61.315 | 24.574 | 1.00 | 30.40 |
| ATOM | 1911 | N | PRO | A | 276 | 66.723 | 61.365 | 23.019 | 1.00 | 27.93 |
| ATOM | 1912 | CD | PRO | A | 276 | 67.267 | 61.693 | 21.677 | 1.00 | 26.71 |
| ATOM | 1913 | CA | PRO | A | 276 | 67.717 | 60.727 | 23.868 | 1.00 | 27.45 |
| ATOM | 1914 | CB | PRO | A | 276 | 68.783 | 60.280 | 22.871 | 1.00 | 27.30 |
| ATOM | 1915 | CG | PRO | A | 276 | 68.711 | 61.288 | 21.774 | 1.00 | 25.15 |
| ATOM | 1916 | C | PRO | A | 276 | 68.307 | 61.745 | 24.845 | 1.00 | 29.86 |
| ATOM | 1917 | O | PRO | A | 276 | 68.482 | 62.925 | 24.497 | 1.00 | 28.23 |
| ATOM | 1918 | N | THR | A | 277 | 68.617 | 61.296 | 26.068 | 1.00 | 28.52 |
| ATOM | 1919 | CA | THR | A | 277 | 69.191 | 62.194 | 27.067 | 1.00 | 28.53 |
| ATOM | 1920 | CB | THR | A | 277 | 68.948 | 61.688 | 28.498 | 1.00 | 27.08 |
| ATOM | 1921 | OG1 | THR | A | 277 | 69.701 | 60.528 | 28.751 | 1.00 | 26.20 |
| ATOM | 1922 | CG2 | THR | A | 277 | 67.504 | 61.380 | 28.816 | 1.00 | 26.96 |
| ATOM | 1923 | C | THR | A | 277 | 70.675 | 62.348 | 26.833 | 1.00 | 29.01 |
| ATOM | 1924 | O | THR | A | 277 | 71.296 | 61.502 | 26.175 | 1.00 | 29.53 |
| ATOM | 1925 | N | ARG | A | 278 | 71.246 | 63.415 | 27.386 | 1.00 | 29.35 |
| ATOM | 1926 | CA | ARG | A | 278 | 72.676 | 63.671 | 27.257 | 1.00 | 31.97 |
| ATOM | 1927 | CB | ARG | A | 278 | 73.041 | 64.986 | 27.950 | 1.00 | 35.10 |
| ATOM | 1928 | CG | ARG | A | 278 | 74.393 | 65.536 | 27.537 | 1.00 | 39.53 |
| ATOM | 1929 | CD | ARG | A | 278 | 75.396 | 65.452 | 28.681 | 1.00 | 44.28 |
| ATOM | 1930 | NE | ARG | A | 278 | 75.067 | 66.386 | 29.763 | 1.00 | 45.67 |
| ATOM | 1931 | CZ | ARG | A | 278 | 75.973 | 67.048 | 30.493 | 1.00 | 47.63 |
| ATOM | 1932 | NH1 | ARG | A | 278 | 77.280 | 66.820 | 30.338 | 1.00 | 48.34 |
| ATOM | 1933 | NH2 | ARG | A | 278 | 75.566 | 67.937 | 31.396 | 1.00 | 48.46 |
| ATOM | 1934 | C | ARG | A | 278 | 73.490 | 62.515 | 27.850 | 1.00 | 31.24 |
| ATOM | 1935 | O | ARG | A | 278 | 74.581 | 62.190 | 27.359 | 1.00 | 32.03 |
| ATOM | 1936 | N | GLU | A | 279 | 72.937 | 61.888 | 28.895 | 1.00 | 29.06 |
| ATOM | 1937 | CA | GLU | A | 279 | 73.574 | 60.749 | 29.555 | 1.00 | 28.36 |
| ATOM | 1938 | CB | GLU | A | 279 | 72.823 | 60.403 | 30.847 | 1.00 | 29.62 |
| ATOM | 1939 | CG | GLU | A | 279 | 73.360 | 59.189 | 31.601 | 1.00 | 33.37 |
| ATOM | 1940 | CD | GLU | A | 279 | 72.382 | 58.696 | 32.669 | 1.00 | 37.46 |
| ATOM | 1941 | OE1 | GLU | A | 279 | 72.139 | 57.462 | 32.746 | 1.00 | 38.36 |
| ATOM | 1942 | OE2 | GLU | A | 279 | 71.849 | 59.544 | 33.423 | 1.00 | 37.26 |
| ATOM | 1943 | C | GLU | A | 279 | 73.581 | 59.537 | 28.620 | 1.00 | 24.06 |
| ATOM | 1944 | O | GLU | A | 279 | 74.582 | 58.865 | 28.468 | 1.00 | 23.16 |
| ATOM | 1945 | N | GLN | A | 280 | 72.457 | 59.281 | 27.984 | 1.00 | 23.18 |
| ATOM | 1946 | CA | GLN | A | 280 | 72.367 | 58.170 | 27.045 | 1.00 | 23.13 |
| ATOM | 1947 | CB | GLN | A | 280 | 70.948 | 58.033 | 26.532 | 1.00 | 21.72 |
| ATOM | 1948 | CG | GLN | A | 280 | 69.976 | 57.424 | 27.513 | 1.00 | 19.01 |
| ATOM | 1949 | CD | GLN | A | 280 | 68.561 | 57.553 | 27.033 | 1.00 | 16.19 |
| ATOM | 1950 | OE1 | GLN | A | 280 | 68.175 | 58.582 | 26.491 | 1.00 | 20.58 |
| ATOM | 1951 | NE2 | GLN | A | 280 | 67.772 | 56.513 | 27.225 | 1.00 | 19.66 |
| ATOM | 1952 | C | GLN | A | 280 | 73.322 | 58.407 | 25.869 | 1.00 | 22.75 |
| ATOM | 1953 | O | GLN | A | 280 | 73.922 | 57.476 | 25.365 | 1.00 | 24.25 |
| ATOM | 1954 | N | ILE | A | 281 | 73.462 | 59.668 | 25.460 | 1.00 | 24.22 |
| ATOM | 1955 | CA | ILE | A | 281 | 74.349 | 60.044 | 24.346 | 1.00 | 26.13 |
| ATOM | 1956 | CB | ILE | A | 281 | 74.150 | 61.514 | 23.913 | 1.00 | 25.19 |
| ATOM | 1957 | CG2 | ILE | A | 281 | 75.294 | 61.981 | 23.010 | 1.00 | 27.25 |
| ATOM | 1958 | CG1 | ILE | A | 281 | 72.804 | 61.683 | 23.216 | 1.00 | 24.20 |
| ATOM | 1959 | CD1 | ILE | A | 281 | 72.269 | 63.097 | 23.280 | 1.00 | 25.74 |
| ATOM | 1960 | C | ILE | A | 281 | 75.817 | 59.824 | 24.663 | 1.00 | 26.96 |
| ATOM | 1961 | O | ILE | A | 281 | 76.585 | 59.418 | 23.783 | 1.00 | 24.09 |
| ATOM | 1962 | N | ARG | A | 282 | 76.220 | 60.108 | 25.915 | 1.00 | 26.63 |
| ATOM | 1963 | CA | ARG | A | 282 | 77.614 | 59.937 | 26.315 | 1.00 | 26.66 |
| ATOM | 1964 | CB | ARG | A | 282 | 77.883 | 60.575 | 27.693 | 1.00 | 30.42 |
| ATOM | 1965 | CG | ARG | A | 282 | 79.344 | 60.496 | 28.132 | 1.00 | 33.94 |
| ATOM | 1966 | CD | ARG | A | 282 | 79.574 | 61.182 | 29.482 | 1.00 | 38.66 |
| ATOM | 1967 | NE | ARG | A | 282 | 80.944 | 60.996 | 29.981 | 1.00 | 41.54 |
| ATOM | 1968 | CZ | ARG | A | 282 | 82.028 | 61.599 | 29.469 | 1.00 | 44.32 |
| ATOM | 1969 | NH1 | ARG | A | 282 | 81.928 | 62.399 | 28.406 | 1.00 | 46.32 |
| ATOM | 1970 | NH2 | ARG | A | 282 | 83.220 | 61.401 | 30.023 | 1.00 | 45.03 |
| ATOM | 1971 | C | ARG | A | 282 | 77.986 | 58.457 | 26.321 | 1.00 | 24.24 |
| ATOM | 1972 | O | ARG | A | 282 | 79.117 | 58.089 | 26.016 | 1.00 | 23.52 |
| ATOM | 1973 | N | GLU | A | 283 | 77.023 | 57.614 | 26.637 | 1.00 | 23.65 |
| ATOM | 1974 | CA | GLU | A | 283 | 77.246 | 56.170 | 26.645 | 1.00 | 25.88 |
| ATOM | 1975 | CB | GLU | A | 283 | 76.023 | 55.468 | 27.224 | 1.00 | 25.65 |
| ATOM | 1976 | CG | GLU | A | 283 | 75.594 | 55.991 | 28.587 | 1.00 | 32.93 |
| ATOM | 1977 | CD | GLU | A | 283 | 76.041 | 55.084 | 29.709 | 1.00 | 33.42 |
| ATOM | 1978 | OE1 | GLU | A | 283 | 75.163 | 54.544 | 30.412 | 1.00 | 35.74 |
| ATOM | 1979 | OE2 | GLU | A | 283 | 77.268 | 54.899 | 29.871 | 1.00 | 34.68 |
| ATOM | 1980 | C | GLU | A | 283 | 77.519 | 55.632 | 25.219 | 1.00 | 26.49 |
| ATOM | 1981 | O | GLU | A | 283 | 78.075 | 54.556 | 25.056 | 1.00 | 26.00 |
| ATOM | 1982 | N | MET | A | 284 | 77.106 | 56.387 | 24.195 | 1.00 | 28.65 |
| ATOM | 1983 | CA | MET | A | 284 | 77.292 | 55.981 | 22.790 | 1.00 | 30.37 |
| ATOM | 1984 | CB | MET | A | 284 | 76.193 | 56.578 | 21.907 | 1.00 | 29.17 |
| ATOM | 1985 | CG | MET | A | 284 | 74.799 | 56.484 | 22.496 | 1.00 | 28.48 |
| ATOM | 1986 | SD | MET | A | 284 | 73.490 | 56.940 | 21.341 | 1.00 | 32.72 |
| ATOM | 1987 | CE | MET | A | 284 | 73.068 | 58.578 | 21.912 | 1.00 | 30.81 |
| ATOM | 1988 | C | MET | A | 284 | 78.662 | 56.353 | 22.236 | 1.00 | 31.62 |
| ATOM | 1989 | O | MET | A | 284 | 78.971 | 55.995 | 21.110 | 1.00 | 32.97 |
| ATOM | 1990 | N | ASN | A | 285 | 79.483 | 57.082 | 23.016 | 1.00 | 33.38 |
| ATOM | 1991 | CA | ASN | A | 285 | 80.818 | 57.508 | 22.563 | 1.00 | 35.76 |
| ATOM | 1992 | CB | ASN | A | 285 | 81.777 | 56.317 | 22.434 | 1.00 | 35.36 |
| ATOM | 1993 | CG | ASN | A | 285 | 81.930 | 55.498 | 23.708 | 1.00 | 37.66 |
| ATOM | 1994 | OD1 | ASN | A | 285 | 82.275 | 54.311 | 23.659 | 1.00 | 35.39 |
| ATOM | 1995 | ND2 | ASN | A | 285 | 81.678 | 56.123 | 24.853 | 1.00 | 36.16 |
| ATOM | 1996 | C | ASN | A | 285 | 80.707 | 58.241 | 21.204 | 1.00 | 37.50 |
| ATOM | 1997 | O | ASN | A | 285 | 81.275 | 57.809 | 20.198 | 1.00 | 37.75 |
| ATOM | 1998 | N | PRO | A | 286 | 79.964 | 59.351 | 21.170 | 1.00 | 38.06 |
| ATOM | 1999 | CD | PRO | A | 286 | 79.255 | 59.943 | 22.329 | 1.00 | 37.89 |
| ATOM | 2000 | CA | PRO | A | 286 | 79.750 | 60.131 | 19.947 | 1.00 | 39.95 |
| ATOM | 2001 | CB | PRO | A | 286 | 78.659 | 61.121 | 20.362 | 1.00 | 39.05 |
| ATOM | 2002 | CG | PRO | A | 286 | 78.886 | 61.302 | 21.826 | 1.00 | 38.06 |
| ATOM | 2003 | C | PRO | A | 286 | 80.974 | 60.911 | 19.486 | 1.00 | 40.66 |
| ATOM | 2004 | O | PRO | A | 286 | 81.759 | 61.425 | 20.291 | 1.00 | 40.65 |
| ATOM | 2005 | N | ASN | A | 287 | 81.103 | 61.051 | 18.171 | 1.00 | 41.08 |
| ATOM | 2006 | CA | ASN | A | 287 | 82.194 | 61.830 | 17.633 | 1.00 | 41.25 |
| ATOM | 2007 | CB | ASN | A | 287 | 82.902 | 61.108 | 16.493 | 1.00 | 43.10 |
| ATOM | 2008 | CG | ASN | A | 287 | 84.105 | 60.323 | 16.998 | 1.00 | 42.68 |
| ATOM | 2009 | OD1 | ASN | A | 287 | 84.162 | 59.101 | 16.876 | 1.00 | 42.74 |
| ATOM | 2010 | ND2 | ASN | A | 287 | 85.074 | 61.027 | 17.573 | 1.00 | 42.61 |
| ATOM | 2011 | C | ASN | A | 287 | 81.687 | 63.222 | 17.269 | 1.00 | 40.62 |
| ATOM | 2012 | O | ASN | A | 287 | 82.456 | 64.141 | 17.021 | 1.00 | 40.65 |
| ATOM | 2013 | N | TYR | A | 288 | 80.367 | 63.359 | 17.310 | 1.00 | 41.10 |
| ATOM | 2014 | CA | TYR | A | 288 | 79.658 | 64.611 | 17.060 | 1.00 | 42.21 |
| ATOM | 2015 | CB | TYR | A | 288 | 79.602 | 65.014 | 15.569 | 1.00 | 42.33 |
| ATOM | 2016 | CG | TYR | A | 288 | 79.418 | 63.880 | 14.588 | 1.00 | 42.43 |
| ATOM | 2017 | CD1 | TYR | A | 288 | 80.507 | 63.341 | 13.914 | 1.00 | 42.88 |
| ATOM | 2018 | CE1 | TYR | A | 288 | 80.351 | 62.306 | 13.006 | 1.00 | 42.57 |
| ATOM | 2019 | CD2 | TYR | A | 288 | 78.159 | 63.354 | 14.326 | 1.00 | 43.59 |
| ATOM | 2020 | CE2 | TYR | A | 288 | 77.993 | 62.319 | 13.420 | 1.00 | 42.81 |
| ATOM | 2021 | CZ | TYR | A | 288 | 79.093 | 61.798 | 12.765 | 1.00 | 42.73 |
| ATOM | 2022 | OH | TYR | A | 288 | 78.934 | 60.750 | 11.876 | 1.00 | 43.10 |
| ATOM | 2023 | C | TYR | A | 288 | 78.262 | 64.512 | 17.645 | 1.00 | 43.00 |
| ATOM | 2024 | O | TYR | A | 288 | 77.644 | 63.450 | 17.612 | 1.00 | 40.24 |
| ATOM | 2025 | N | THR | A | 289 | 77.776 | 65.611 | 18.203 | 1.00 | 44.37 |
| ATOM | 2026 | CA | THR | A | 289 | 76.451 | 65.614 | 18.812 | 1.00 | 47.10 |
| ATOM | 2027 | CB | THR | A | 289 | 76.541 | 65.473 | 20.347 | 1.00 | 46.92 |
| ATOM | 2028 | OG1 | THR | A | 289 | 76.811 | 66.725 | 20.970 | 1.00 | 45.80 |
| ATOM | 2029 | CG2 | THR | A | 289 | 77.581 | 64.474 | 20.816 | 1.00 | 45.35 |
| ATOM | 2030 | C | THR | A | 289 | 75.666 | 66.861 | 18.435 | 1.00 | 48.71 |
| ATOM | 2031 | O | THR | A | 289 | 74.506 | 66.994 | 18.816 | 1.00 | 49.12 |
| ATOM | 2032 | N | GLU | A | 290 | 76.307 | 67.767 | 17.687 | 1.00 | 50.41 |
| ATOM | 2033 | CA | GLU | A | 290 | 75.674 | 69.008 | 17.251 | 1.00 | 52.56 |
| ATOM | 2034 | CB | GLU | A | 290 | 76.706 | 69.963 | 16.653 | 1.00 | 54.21 |
| ATOM | 2035 | CG | GLU | A | 290 | 77.852 | 70.307 | 17.595 | 1.00 | 56.44 |
| ATOM | 2036 | CD | GLU | A | 290 | 79.112 | 69.509 | 17.307 | 1.00 | 58.31 |
| ATOM | 2037 | OE1 | GLU | A | 290 | 80.205 | 70.115 | 17.278 | 1.00 | 59.38 |
| ATOM | 2038 | OE2 | GLU | A | 290 | 79.009 | 68.278 | 17.113 | 1.00 | 58.91 |
| ATOM | 2039 | C | GLU | A | 290 | 74.556 | 68.733 | 16.251 | 1.00 | 53.63 |
| ATOM | 2040 | O | GLU | A | 290 | 74.504 | 69.325 | 15.176 | 1.00 | 53.39 |
| ATOM | 2041 | N | PHE | A | 291 | 73.665 | 67.829 | 16.627 | 1.00 | 54.78 |
| ATOM | 2042 | CA | PHE | A | 291 | 72.535 | 67.445 | 15.809 | 1.00 | 56.14 |
| ATOM | 2043 | CB | PHE | A | 291 | 72.090 | 66.025 | 16.169 | 1.00 | 56.00 |
| ATOM | 2044 | CG | PHE | A | 291 | 72.958 | 64.947 | 15.603 | 1.00 | 56.83 |
| ATOM | 2045 | CD1 | PHE | A | 291 | 73.985 | 64.405 | 16.353 | 1.00 | 56.77 |
| ATOM | 2046 | CD2 | PHE | A | 291 | 72.740 | 64.467 | 14.321 | 1.00 | 57.19 |
| ATOM | 2047 | CE1 | PHE | A | 291 | 74.781 | 63.401 | 15.836 | 1.00 | 57.29 |
| ATOM | 2048 | CE2 | PHE | A | 291 | 73.533 | 63.464 | 13.799 | 1.00 | 57.70 |
| ATOM | 2049 | CZ | PHE | A | 291 | 74.556 | 62.930 | 14.558 | 1.00 | 57.44 |
| ATOM | 2050 | C | PHE | A | 291 | 71.376 | 68.409 | 16.018 | 1.00 | 57.37 |
| ATOM | 2051 | O | PHE | A | 291 | 71.563 | 69.557 | 16.413 | 1.00 | 58.88 |
| ATOM | 2052 | N | LYS | A | 292 | 70.180 | 67.931 | 15.748 | 1.00 | 57.76 |
| ATOM | 2053 | CA | LYS | A | 292 | 68.979 | 68.726 | 15.893 | 1.00 | 58.17 |
| ATOM | 2054 | CB | LYS | A | 292 | 68.738 | 69.566 | 14.628 | 1.00 | 60.13 |
| ATOM | 2055 | CG | LYS | A | 292 | 69.554 | 69.120 | 13.402 | 1.00 | 61.32 |
| ATOM | 2056 | CD | LYS | A | 292 | 69.445 | 67.616 | 13.137 | 1.00 | 61.28 |
| ATOM | 2057 | CE | LYS | A | 292 | 68.585 | 67.313 | 11.926 | 1.00 | 61.42 |
| ATOM | 2058 | NZ | LYS | A | 292 | 67.875 | 66.014 | 12.071 | 1.00 | 62.17 |
| ATOM | 2059 | C | LYS | A | 292 | 67.804 | 67.798 | 16.171 | 1.00 | 57.78 |
| ATOM | 2060 | O | LYS | A | 292 | 67.150 | 67.291 | 15.252 | 1.00 | 58.28 |
| ATOM | 2061 | N | PHE | A | 293 | 67.575 | 67.541 | 17.451 | 1.00 | 56.83 |
| ATOM | 2062 | CA | PHE | A | 293 | 66.516 | 66.640 | 17.871 | 1.00 | 55.06 |
| ATOM | 2063 | CB | PHE | A | 293 | 66.832 | 66.024 | 19.244 | 1.00 | 54.49 |
| ATOM | 2064 | CG | PHE | A | 293 | 68.277 | 65.646 | 19.440 | 1.00 | 52.66 |
| ATOM | 2065 | CD1 | PHE | A | 293 | 68.885 | 64.716 | 18.615 | 1.00 | 52.89 |
| ATOM | 2066 | CD2 | PHE | A | 293 | 69.026 | 66.224 | 20.454 | 1.00 | 51.93 |
| ATOM | 2067 | CE1 | PHE | A | 293 | 70.213 | 64.368 | 18.795 | 1.00 | 52.57 |
| ATOM | 2068 | CE2 | PHE | A | 293 | 70.354 | 65.883 | 20.641 | 1.00 | 52.26 |
| ATOM | 2069 | CZ | PHE | A | 293 | 70.949 | 64.952 | 19.809 | 1.00 | 52.07 |
| ATOM | 2070 | C | PHE | A | 293 | 65.165 | 67.340 | 17.888 | 1.00 | 54.04 |
| ATOM | 2071 | O | PHE | A | 293 | 65.037 | 68.457 | 18.391 | 1.00 | 53.67 |
| ATOM | 2072 | N | PRO | A | 294 | 64.136 | 66.688 | 17.317 | 1.00 | 53.78 |
| ATOM | 2073 | CD | PRO | A | 294 | 64.223 | 65.365 | 16.670 | 1.00 | 53.01 |
| ATOM | 2074 | CA | PRO | A | 294 | 62.779 | 67.248 | 17.254 | 1.00 | 53.38 |
| ATOM | 2075 | CB | PRO | A | 294 | 62.006 | 66.233 | 16.401 | 1.00 | 53.26 |
| ATOM | 2076 | CG | PRO | A | 294 | 62.786 | 64.967 | 16.496 | 1.00 | 53.31 |
| ATOM | 2077 | C | PRO | A | 294 | 62.117 | 67.413 | 18.635 | 1.00 | 52.79 |
| ATOM | 2078 | O | PRO | A | 294 | 61.031 | 67.995 | 18.742 | 1.00 | 53.69 |
| ATOM | 2079 | N | GLN | A | 295 | 62.766 | 66.901 | 19.684 | 1.00 | 51.09 |
| ATOM | 2080 | CA | GLN | A | 295 | 62.235 | 66.989 | 21.053 | 1.00 | 49.40 |
| ATOM | 2081 | CB | GLN | A | 295 | 62.135 | 68.440 | 21.532 | 1.00 | 47.56 |
| ATOM | 2082 | CG | GLN | A | 295 | 63.474 | 69.148 | 21.658 | 1.00 | 46.71 |
| ATOM | 2083 | CD | GLN | A | 295 | 64.518 | 68.322 | 22.382 | 1.00 | 45.57 |
| ATOM | 2084 | OE1 | GLN | A | 295 | 64.234 | 67.695 | 23.393 | 1.00 | 45.44 |
| ATOM | 2085 | NE2 | GLN | A | 295 | 65.737 | 68.324 | 21.868 | 1.00 | 45.50 |
| ATOM | 2086 | C | GLN | A | 295 | 60.888 | 66.285 | 21.173 | 1.00 | 48.71 |
| ATOM | 2087 | O | GLN | A | 295 | 59.828 | 66.920 | 21.166 | 1.00 | 48.11 |
| ATOM | 2088 | N | ILE | A | 296 | 60.944 | 64.961 | 21.274 | 1.00 | 47.95 |
| ATOM | 2089 | CA | ILE | A | 296 | 59.745 | 64.145 | 21.384 | 1.00 | 47.97 |
| ATOM | 2090 | CB | ILE | A | 296 | 59.590 | 63.210 | 20.160 | 1.00 | 47.45 |
| ATOM | 2091 | CG2 | ILE | A | 296 | 58.156 | 62.723 | 20.041 | 1.00 | 47.21 |
| ATOM | 2092 | CG1 | ILE | A | 296 | 60.015 | 63.927 | 18.873 | 1.00 | 47.88 |
| ATOM | 2093 | CD1 | ILE | A | 296 | 60.015 | 63.036 | 17.649 | 1.00 | 48.40 |
| ATOM | 2094 | C | ILE | A | 296 | 59.765 | 63.295 | 22.652 | 1.00 | 47.76 |
| ATOM | 2095 | O | ILE | A | 296 | 60.830 | 63.005 | 23.208 | 1.00 | 48.33 |
| ATOM | 2096 | N | LYS | A | 297 | 58.579 | 62.877 | 23.084 | 1.00 | 47.32 |
| ATOM | 2097 | CA | LYS | A | 297 | 58.441 | 62.032 | 24.264 | 1.00 | 46.79 |
| ATOM | 2098 | CB | LYS | A | 297 | 57.278 | 62.505 | 25.171 | 1.00 | 48.16 |
| ATOM | 2099 | CG | LYS | A | 297 | 56.174 | 63.305 | 24.466 | 1.00 | 48.76 |
| ATOM | 2100 | CD | LYS | A | 297 | 56.550 | 64.778 | 24.251 | 1.00 | 50.38 |
| ATOM | 2101 | CE | LYS | A | 297 | 56.720 | 65.542 | 25.569 | 1.00 | 50.43 |
| ATOM | 2102 | NZ | LYS | A | 297 | 57.428 | 66.847 | 25.376 | 1.00 | 49.71 |
| ATOM | 2103 | C | LYS | A | 297 | 58.234 | 60.584 | 23.819 | 1.00 | 46.40 |
| ATOM | 2104 | O | LYS | A | 297 | 57.437 | 60.318 | 22.917 | 1.00 | 46.93 |
| ATOM | 2105 | N | ALA | A | 298 | 58.980 | 59.659 | 24.427 | 1.00 | 45.46 |
| ATOM | 2106 | CA | ALA | A | 298 | 58.911 | 58.237 | 24.079 | 1.00 | 45.16 |
| ATOM | 2107 | CB | ALA | A | 298 | 59.583 | 57.377 | 25.139 | 1.00 | 46.44 |
| ATOM | 2108 | C | ALA | A | 298 | 57.494 | 57.744 | 23.819 | 1.00 | 44.68 |
| ATOM | 2109 | O | ALA | A | 298 | 56.558 | 58.075 | 24.546 | 1.00 | 44.42 |
| ATOM | 2110 | N | HIS | A | 299 | 57.357 | 56.923 | 22.782 | 1.00 | 44.68 |
| ATOM | 2111 | CA | HIS | A | 299 | 56.075 | 56.332 | 22.411 | 1.00 | 44.10 |
| ATOM | 2112 | CB | HIS | A | 299 | 55.975 | 56.186 | 20.878 | 1.00 | 44.34 |
| ATOM | 2113 | CG | HIS | A | 299 | 54.587 | 55.926 | 20.361 | 1.00 | 43.08 |
| ATOM | 2114 | CD2 | HIS | A | 299 | 54.047 | 54.824 | 19.788 | 1.00 | 43.51 |
| ATOM | 2115 | ND1 | HIS | A | 299 | 53.574 | 56.864 | 20.399 | 1.00 | 43.83 |
| ATOM | 2116 | CE1 | HIS | A | 299 | 52.474 | 56.351 | 19.878 | 1.00 | 43.22 |
| ATOM | 2117 | NE2 | HIS | A | 299 | 52.734 | 55.113 | 19.499 | 1.00 | 42.58 |
| ATOM | 2118 | C | HIS | A | 299 | 55.959 | 54.963 | 23.078 | 1.00 | 44.10 |
| ATOM | 2119 | O | HIS | A | 299 | 56.782 | 54.080 | 22.838 | 1.00 | 44.94 |
| ATOM | 2120 | N | PRO | A | 300 | 54.944 | 54.769 | 23.936 | 1.00 | 43.53 |
| ATOM | 2121 | CD | PRO | A | 300 | 53.921 | 55.772 | 24.283 | 1.00 | 43.05 |
| ATOM | 2122 | CA | PRO | A | 300 | 54.737 | 53.501 | 24.649 | 1.00 | 42.90 |
| ATOM | 2123 | CB | PRO | A | 300 | 53.286 | 53.614 | 25.116 | 1.00 | 43.38 |
| ATOM | 2124 | CG | PRO | A | 300 | 53.090 | 55.076 | 25.330 | 1.00 | 43.66 |
| ATOM | 2125 | C | PRO | A | 300 | 54.911 | 52.287 | 23.741 | 1.00 | 42.52 |
| ATOM | 2126 | O | PRO | A | 300 | 54.217 | 52.152 | 22.735 | 1.00 | 42.26 |
| ATOM | 2127 | N | TRP | A | 301 | 55.846 | 51.410 | 24.103 | 1.00 | 42.18 |
| ATOM | 2128 | CA | TRP | A | 301 | 56.120 | 50.207 | 23.322 | 1.00 | 42.14 |
| ATOM | 2129 | CB | TRP | A | 301 | 57.165 | 49.330 | 24.015 | 1.00 | 41.33 |
| ATOM | 2130 | CG | TRP | A | 301 | 58.583 | 49.696 | 23.682 | 1.00 | 40.34 |
| ATOM | 2131 | CD2 | TRP | A | 301 | 59.190 | 49.696 | 22.380 | 1.00 | 39.60 |
| ATOM | 2132 | CE2 | TRP | A | 301 | 60.527 | 50.110 | 22.543 | 1.00 | 39.49 |
| ATOM | 2133 | CE3 | TRP | A | 301 | 58.731 | 49.387 | 21.095 | 1.00 | 39.48 |
| ATOM | 2134 | CD1 | TRP | A | 301 | 59.556 | 50.098 | 24.556 | 1.00 | 40.21 |
| ATOM | 2135 | NE1 | TRP | A | 301 | 60.726 | 50.351 | 23.879 | 1.00 | 39.18 |
| ATOM | 2136 | CZ2 | TRP | A | 301 | 61.407 | 50.220 | 21.470 | 1.00 | 39.55 |
| ATOM | 2137 | CZ3 | TRP | A | 301 | 59.604 | 49.497 | 20.033 | 1.00 | 38.40 |
| ATOM | 2138 | CH2 | TRP | A | 301 | 60.925 | 49.910 | 20.224 | 1.00 | 39.52 |
| ATOM | 2139 | C | TRP | A | 301 | 54.852 | 49.407 | 23.052 | 1.00 | 43.11 |
| ATOM | 2140 | O | TRP | A | 301 | 54.690 | 48.830 | 21.975 | 1.00 | 43.94 |
| ATOM | 2141 | N | THR | A | 302 | 53.953 | 49.375 | 24.032 | 1.00 | 44.41 |
| ATOM | 2142 | CA | THR | A | 302 | 52.692 | 48.643 | 23.901 | 1.00 | 45.14 |
| ATOM | 2143 | CB | THR | A | 302 | 51.997 | 48.489 | 25.259 | 1.00 | 45.40 |
| ATOM | 2144 | OG1 | THR | A | 302 | 50.806 | 47.733 | 25.138 | 1.00 | 47.13 |
| ATOM | 2145 | CG2 | THR | A | 302 | 51.662 | 49.797 | 25.943 | 1.00 | 45.17 |
| ATOM | 2146 | C | THR | A | 302 | 51.763 | 49.282 | 22.860 | 1.00 | 45.41 |
| ATOM | 2147 | O | THR | A | 302 | 50.902 | 48.608 | 22.286 | 1.00 | 46.19 |
| ATOM | 2148 | N | LYS | A | 303 | 51.964 | 50.573 | 22.599 | 1.00 | 44.91 |
| ATOM | 2149 | CA | LYS | A | 303 | 51.172 | 51.295 | 21.607 | 1.00 | 44.23 |
| ATOM | 2150 | CB | LYS | A | 303 | 50.962 | 52.747 | 22.053 | 1.00 | 45.50 |
| ATOM | 2151 | CG | LYS | A | 303 | 49.656 | 52.991 | 22.792 | 1.00 | 47.39 |
| ATOM | 2152 | CD | LYS | A | 303 | 49.381 | 54.483 | 22.937 | 1.00 | 49.70 |
| ATOM | 2153 | CE | LYS | A | 303 | 48.426 | 54.994 | 21.863 | 1.00 | 50.84 |
| ATOM | 2154 | NZ | LYS | A | 303 | 47.185 | 55.585 | 22.445 | 1.00 | 51.69 |
| ATOM | 2155 | C | LYS | A | 303 | 51.862 | 51.272 | 20.227 | 1.00 | 43.47 |
| ATOM | 2156 | O | LYS | A | 303 | 51.232 | 51.562 | 19.212 | 1.00 | 42.78 |
| ATOM | 2157 | N | VAL | A | 304 | 53.158 | 50.936 | 20.199 | 1.00 | 42.34 |
| ATOM | 2158 | CA | VAL | A | 304 | 53.917 | 50.891 | 18.943 | 1.00 | 42.40 |
| ATOM | 2159 | CB | VAL | A | 304 | 55.442 | 50.779 | 19.184 | 1.00 | 42.11 |
| ATOM | 2160 | CG1 | VAL | A | 304 | 56.177 | 50.320 | 17.921 | 1.00 | 40.82 |
| ATOM | 2161 | CG2 | VAL | A | 304 | 56.002 | 52.102 | 19.681 | 1.00 | 39.70 |
| ATOM | 2162 | C | VAL | A | 304 | 53.450 | 49.748 | 18.042 | 1.00 | 42.22 |
| ATOM | 2163 | O | VAL | A | 304 | 53.406 | 49.890 | 16.821 | 1.00 | 43.06 |
| ATOM | 2164 | N | PHE | A | 305 | 53.106 | 48.617 | 18.652 | 1.00 | 41.61 |
| ATOM | 2165 | CA | PHE | A | 305 | 52.649 | 47.452 | 17.906 | 1.00 | 41.24 |
| ATOM | 2166 | CB | PHE | A | 305 | 53.269 | 46.176 | 18.472 | 1.00 | 37.57 |
| ATOM | 2167 | CG | PHE | A | 305 | 54.763 | 46.198 | 18.469 | 1.00 | 35.03 |
| ATOM | 2168 | CD1 | PHE | A | 305 | 55.463 | 46.597 | 19.598 | 1.00 | 33.16 |
| ATOM | 2169 | CD2 | PHE | A | 305 | 55.470 | 45.834 | 17.335 | 1.00 | 33.57 |
| ATOM | 2170 | CE1 | PHE | A | 305 | 56.838 | 46.632 | 19.597 | 1.00 | 31.36 |
| ATOM | 2171 | CE2 | PHE | A | 305 | 56.850 | 45.865 | 17.328 | 1.00 | 32.96 |
| ATOM | 2172 | CZ | PHE | A | 305 | 57.534 | 46.265 | 18.462 | 1.00 | 33.78 |
| ATOM | 2173 | C | PHE | A | 305 | 51.135 | 47.345 | 17.901 | 1.00 | 42.81 |
| ATOM | 2174 | O | PHE | A | 305 | 50.455 | 47.939 | 18.738 | 1.00 | 44.77 |
| ATOM | 2175 | N | ARG | A | 306 | 50.606 | 46.579 | 16.955 | 1.00 | 44.27 |
| ATOM | 2176 | CA | ARG | A | 306 | 49.169 | 46.398 | 16.859 | 1.00 | 45.78 |
| ATOM | 2177 | CB | ARG | A | 306 | 48.788 | 45.720 | 15.541 | 1.00 | 47.80 |
| ATOM | 2178 | CG | ARG | A | 306 | 49.444 | 44.366 | 15.315 | 1.00 | 50.54 |
| ATOM | 2179 | CD | ARG | A | 306 | 49.697 | 44.111 | 13.829 | 1.00 | 52.93 |
| ATOM | 2180 | NE | ARG | A | 306 | 48.876 | 43.013 | 13.315 | 1.00 | 55.30 |
| ATOM | 2181 | CZ | ARG | A | 306 | 49.028 | 41.731 | 13.669 | 1.00 | 56.25 |
| ATOM | 2182 | NH1 | ARG | A | 306 | 49.967 | 41.375 | 14.547 | 1.00 | 55.27 |
| ATOM | 2183 | NH2 | ARG | A | 306 | 48.233 | 40.801 | 13.143 | 1.00 | 56.50 |
| ATOM | 2184 | C | ARG | A | 306 | 48.663 | 45.589 | 18.049 | 1.00 | 45.74 |
| ATOM | 2185 | O | ARG | A | 306 | 49.404 | 44.793 | 18.619 | 1.00 | 45.14 |
| ATOM | 2186 | N | PRO | A | 307 | 47.397 | 45.793 | 18.453 | 1.00 | 46.23 |
| ATOM | 2187 | CD | PRO | A | 307 | 46.442 | 46.735 | 17.846 | 1.00 | 46.07 |
| ATOM | 2188 | CA | PRO | A | 307 | 46.811 | 45.082 | 19.592 | 1.00 | 46.18 |
| ATOM | 2189 | CB | PRO | A | 307 | 45.340 | 45.530 | 19.597 | 1.00 | 46.66 |
| ATOM | 2190 | CG | PRO | A | 307 | 45.115 | 46.175 | 18.269 | 1.00 | 46.15 |
| ATOM | 2191 | C | PRO | A | 307 | 46.909 | 43.567 | 19.444 | 1.00 | 46.73 |
| ATOM | 2192 | O | PRO | A | 307 | 47.062 | 43.049 | 18.339 | 1.00 | 47.71 |
| ATOM | 2193 | N | ARG | A | 308 | 46.820 | 42.870 | 20.574 | 1.00 | 46.74 |
| ATOM | 2194 | CA | ARG | A | 308 | 46.889 | 41.411 | 20.605 | 1.00 | 46.91 |
| ATOM | 2195 | CB | ARG | A | 308 | 45.830 | 40.797 | 19.674 | 1.00 | 49.70 |
| ATOM | 2196 | CG | ARG | A | 308 | 44.566 | 40.340 | 20.406 | 1.00 | 52.99 |
| ATOM | 2197 | CD | ARG | A | 308 | 44.655 | 38.885 | 20.869 | 1.00 | 57.02 |
| ATOM | 2198 | NE | ARG | A | 308 | 45.943 | 38.578 | 21.513 | 1.00 | 60.77 |
| ATOM | 2199 | CZ | ARG | A | 308 | 46.106 | 38.320 | 22.825 | 1.00 | 61.29 |
| ATOM | 2200 | NH1 | ARG | A | 308 | 45.061 | 38.310 | 23.659 | 1.00 | 61.76 |
| ATOM | 2201 | NH2 | ARG | A | 308 | 47.326 | 38.070 | 23.300 | 1.00 | 61.20 |
| ATOM | 2202 | C | ARG | A | 308 | 48.292 | 40.881 | 20.274 | 1.00 | 44.79 |
| ATOM | 2203 | O | ARG | A | 308 | 48.471 | 39.680 | 20.055 | 1.00 | 43.82 |
| ATOM | 2204 | N | THR | A | 309 | 49.294 | 41.760 | 20.285 | 1.00 | 42.99 |
| ATOM | 2205 | CA | THR | A | 309 | 50.675 | 41.332 | 20.030 | 1.00 | 41.54 |
| ATOM | 2206 | CB | THR | A | 309 | 51.547 | 42.495 | 19.525 | 1.00 | 39.98 |
| ATOM | 2207 | OG1 | THR | A | 309 | 51.117 | 42.936 | 18.253 | 1.00 | 39.67 |
| ATOM | 2208 | CG2 | THR | A | 309 | 53.022 | 42.166 | 19.418 | 1.00 | 38.09 |
| ATOM | 2209 | C | THR | A | 309 | 51.258 | 40.740 | 21.315 | 1.00 | 40.62 |
| ATOM | 2210 | O | THR | A | 309 | 51.132 | 41.336 | 22.382 | 1.00 | 41.53 |
| ATOM | 2211 | N | PRO | A | 310 | 51.883 | 39.551 | 21.238 | 1.00 | 39.36 |
| ATOM | 2212 | CD | PRO | A | 310 | 52.065 | 38.755 | 20.008 | 1.00 | 38.76 |
| ATOM | 2213 | CA | PRO | A | 310 | 52.461 | 38.881 | 22.415 | 1.00 | 38.43 |
| ATOM | 2214 | CB | PRO | A | 310 | 53.390 | 37.843 | 21.792 | 1.00 | 37.91 |
| ATOM | 2215 | CG | PRO | A | 310 | 52.727 | 37.494 | 20.500 | 1.00 | 39.30 |
| ATOM | 2216 | C | PRO | A | 310 | 53.247 | 39.846 | 23.309 | 1.00 | 36.53 |
| ATOM | 2217 | O | PRO | A | 310 | 54.293 | 40.349 | 22.918 | 1.00 | 37.72 |
| ATOM | 2218 | N | PRO | A | 311 | 52.744 | 40.113 | 24.530 | 1.00 | 34.81 |
| ATOM | 2219 | CD | PRO | A | 311 | 51.498 | 39.546 | 25.078 | 1.00 | 34.82 |
| ATOM | 2220 | CA | PRO | A | 311 | 53.400 | 41.021 | 25.487 | 1.00 | 32.04 |
| ATOM | 2221 | CB | PRO | A | 311 | 52.619 | 40.791 | 26.779 | 1.00 | 33.08 |
| ATOM | 2222 | CG | PRO | A | 311 | 51.272 | 40.357 | 26.321 | 1.00 | 35.07 |
| ATOM | 2223 | C | PRO | A | 311 | 54.878 | 40.685 | 25.682 | 1.00 | 29.08 |
| ATOM | 2224 | O | PRO | A | 311 | 55.706 | 41.577 | 25.833 | 1.00 | 28.54 |
| ATOM | 2225 | N | GLU | A | 312 | 55.202 | 39.396 | 25.647 | 1.00 | 28.12 |
| ATOM | 2226 | CA | GLU | A | 312 | 56.578 | 38.937 | 25.791 | 1.00 | 28.15 |
| ATOM | 2227 | CB | GLU | A | 312 | 56.624 | 37.416 | 25.989 | 1.00 | 32.38 |
| ATOM | 2228 | CG | GLU | A | 312 | 55.811 | 36.917 | 27.186 | 1.00 | 38.17 |
| ATOM | 2229 | CD | GLU | A | 312 | 54.382 | 36.502 | 26.839 | 1.00 | 40.82 |
| ATOM | 2230 | OE1 | GLU | A | 312 | 53.864 | 35.572 | 27.490 | 1.00 | 43.31 |
| ATOM | 2231 | OE2 | GLU | A | 312 | 53.775 | 37.105 | 25.923 | 1.00 | 42.33 |
| ATOM | 2232 | C | GLU | A | 312 | 57.434 | 39.373 | 24.586 | 1.00 | 26.26 |
| ATOM | 2233 | O | GLU | A | 312 | 58.625 | 39.636 | 24.729 | 1.00 | 25.86 |
| ATOM | 2234 | N | ALA | A | 313 | 56.805 | 39.489 | 23.407 | 1.00 | 24.44 |
| ATOM | 2235 | CA | ALA | A | 313 | 57.502 | 39.945 | 22.188 | 1.00 | 24.09 |
| ATOM | 2236 | CB | ALA | A | 313 | 56.615 | 39.725 | 20.955 | 1.00 | 22.36 |
| ATOM | 2237 | C | ALA | A | 313 | 57.835 | 41.425 | 22.332 | 1.00 | 22.39 |
| ATOM | 2238 | O | ALA | A | 313 | 58.938 | 41.864 | 22.034 | 1.00 | 23.16 |
| ATOM | 2239 | N | ILE | A | 314 | 56.871 | 42.183 | 22.835 | 1.00 | 22.65 |
| ATOM | 2240 | CA | ILE | A | 314 | 57.047 | 43.610 | 23.076 | 1.00 | 23.93 |
| ATOM | 2241 | CB | ILE | A | 314 | 55.708 | 44.254 | 23.514 | 1.00 | 24.11 |
| ATOM | 2242 | CG2 | ILE | A | 314 | 55.833 | 45.766 | 23.637 | 1.00 | 27.02 |
| ATOM | 2243 | CG1 | ILE | A | 314 | 54.597 | 43.894 | 22.527 | 1.00 | 25.37 |
| ATOM | 2244 | CD1 | ILE | A | 314 | 53.285 | 44.616 | 22.774 | 1.00 | 23.59 |
| ATOM | 2245 | C | ILE | A | 314 | 58.118 | 43.841 | 24.149 | 1.00 | 23.96 |
| ATOM | 2246 | O | ILE | A | 314 | 58.948 | 44.748 | 24.043 | 1.00 | 25.44 |
| ATOM | 2247 | N | ALA | A | 315 | 58.114 | 43.002 | 25.190 | 1.00 | 24.48 |
| ATOM | 2248 | CA | ALA | A | 315 | 59.101 | 43.127 | 26.275 | 1.00 | 21.38 |
| ATOM | 2249 | CB | ALA | A | 315 | 58.794 | 42.130 | 27.378 | 1.00 | 18.99 |
| ATOM | 2250 | C | ALA | A | 315 | 60.525 | 42.939 | 25.753 | 1.00 | 20.50 |
| ATOM | 2251 | O | ALA | A | 315 | 61.425 | 43.723 | 26.078 | 1.00 | 18.67 |
| ATOM | 2252 | N | LEU | A | 316 | 60.725 | 41.892 | 24.928 | 1.00 | 19.20 |
| ATOM | 2253 | CA | LEU | A | 316 | 62.045 | 41.609 | 24.344 | 1.00 | 19.31 |
| ATOM | 2254 | CB | LEU | A | 316 | 62.010 | 40.265 | 23.565 | 1.00 | 19.88 |
| ATOM | 2255 | CG | LEU | A | 316 | 63.240 | 39.902 | 22.709 | 1.00 | 17.67 |
| ATOM | 2256 | CD1 | LEU | A | 316 | 64.464 | 39.652 | 23.567 | 1.00 | 15.53 |
| ATOM | 2257 | CD2 | LEU | A | 316 | 62.956 | 38.685 | 21.848 | 1.00 | 17.95 |
| ATOM | 2258 | C | LEU | A | 316 | 62.510 | 42.792 | 23.466 | 1.00 | 18.37 |
| ATOM | 2259 | O | LEU | A | 316 | 63.686 | 43.175 | 23.480 | 1.00 | 18.43 |
| ATOM | 2260 | N | CYS | A | 317 | 61.566 | 43.396 | 22.739 | 1.00 | 18.93 |
| ATOM | 2261 | CA | CYS | A | 317 | 61.852 | 44.557 | 21.884 | 1.00 | 22.24 |
| ATOM | 2262 | CB | CYS | A | 317 | 60.592 | 44.975 | 21.114 | 1.00 | 25.99 |
| ATOM | 2263 | SG | CYS | A | 317 | 60.621 | 44.562 | 19.353 | 1.00 | 36.98 |
| ATOM | 2264 | C | CYS | A | 317 | 62.384 | 45.751 | 22.681 | 1.00 | 22.08 |
| ATOM | 2265 | O | CYS | A | 317 | 63.337 | 46.422 | 22.253 | 1.00 | 18.81 |
| ATOM | 2266 | N | SER | A | 318 | 61.761 | 46.029 | 23.849 | 1.00 | 21.02 |
| ATOM | 2267 | CA | SER | A | 318 | 62.199 | 47.159 | 24.695 | 1.00 | 19.59 |
| ATOM | 2268 | CB | SER | A | 318 | 61.212 | 47.439 | 25.856 | 1.00 | 23.13 |
| ATOM | 2269 | OG | SER | A | 318 | 60.959 | 46.274 | 26.641 | 1.00 | 19.46 |
| ATOM | 2270 | C | SER | A | 318 | 63.621 | 46.960 | 25.222 | 1.00 | 18.44 |
| ATOM | 2271 | O | SER | A | 318 | 64.365 | 47.920 | 25.386 | 1.00 | 18.50 |
| ATOM | 2272 | N | ARG | A | 319 | 64.001 | 45.708 | 25.480 | 1.00 | 19.95 |
| ATOM | 2273 | CA | ARG | A | 319 | 65.337 | 45.401 | 25.997 | 1.00 | 21.38 |
| ATOM | 2274 | CB | ARG | A | 319 | 65.293 | 44.140 | 26.861 | 1.00 | 23.27 |
| ATOM | 2275 | CG | ARG | A | 319 | 64.261 | 44.200 | 27.983 | 1.00 | 25.35 |
| ATOM | 2276 | CD | ARG | A | 319 | 64.580 | 45.302 | 28.979 | 1.00 | 23.98 |
| ATOM | 2277 | NE | ARG | A | 319 | 65.984 | 45.280 | 29.403 | 1.00 | 28.61 |
| ATOM | 2278 | CZ | ARG | A | 319 | 66.559 | 44.274 | 30.092 | 1.00 | 27.11 |
| ATOM | 2279 | NH1 | ARG | A | 319 | 65.851 | 43.219 | 30.478 | 1.00 | 27.95 |
| ATOM | 2280 | NH2 | ARG | A | 319 | 67.849 | 44.335 | 30.397 | 1.00 | 28.62 |
| ATOM | 2281 | C | ARG | A | 319 | 66.403 | 45.239 | 24.899 | 1.00 | 22.00 |
| ATOM | 2282 | O | ARG | A | 319 | 67.574 | 45.011 | 25.217 | 1.00 | 21.44 |
| ATOM | 2283 | N | LEU | A | 320 | 66.001 | 45.364 | 23.620 | 1.00 | 20.51 |
| ATOM | 2284 | CA | LEU | A | 320 | 66.947 | 45.248 | 22.481 | 1.00 | 18.20 |
| ATOM | 2285 | CB | LEU | A | 320 | 66.417 | 44.282 | 21.390 | 1.00 | 15.82 |
| ATOM | 2286 | CG | LEU | A | 320 | 66.077 | 42.864 | 21.851 | 1.00 | 13.56 |
| ATOM | 2287 | CD1 | LEU | A | 320 | 65.158 | 42.168 | 20.860 | 1.00 | 12.01 |
| ATOM | 2288 | CD2 | LEU | A | 320 | 67.332 | 42.048 | 22.104 | 1.00 | 14.30 |
| ATOM | 2289 | C | LEU | A | 320 | 67.167 | 46.613 | 21.886 | 1.00 | 14.55 |
| ATOM | 2290 | O | LEU | A | 320 | 68.285 | 47.015 | 21.649 | 1.00 | 17.73 |
| ATOM | 2291 | N | LEU | A | 321 | 66.074 | 47.332 | 21.680 | 1.00 | 15.67 |
| ATOM | 2292 | CA | LEU | A | 321 | 66.126 | 48.680 | 21.144 | 1.00 | 17.61 |
| ATOM | 2293 | CB | LEU | A | 321 | 64.892 | 48.978 | 20.278 | 1.00 | 15.07 |
| ATOM | 2294 | CG | LEU | A | 321 | 64.632 | 47.982 | 19.146 | 1.00 | 17.45 |
| ATOM | 2295 | CD1 | LEU | A | 321 | 63.217 | 48.144 | 18.622 | 1.00 | 12.07 |
| ATOM | 2296 | CD2 | LEU | A | 321 | 65.674 | 48.164 | 18.038 | 1.00 | 11.94 |
| ATOM | 2297 | C | LEU | A | 321 | 66.266 | 49.730 | 22.255 | 1.00 | 17.32 |
| ATOM | 2298 | O | LEU | A | 321 | 65.358 | 50.514 | 22.500 | 1.00 | 15.83 |
| ATOM | 2299 | N | GLU | A | 322 | 67.424 | 49.746 | 22.897 | 1.00 | 20.87 |
| ATOM | 2300 | CA | GLU | A | 322 | 67.708 | 50.697 | 23.970 | 1.00 | 21.68 |
| ATOM | 2301 | CB | GLU | A | 322 | 68.114 | 49.931 | 25.246 | 1.00 | 22.81 |
| ATOM | 2302 | CG | GLU | A | 322 | 67.688 | 50.614 | 26.542 | 1.00 | 28.40 |
| ATOM | 2303 | CD | GLU | A | 322 | 66.652 | 49.815 | 27.315 | 1.00 | 28.36 |
| ATOM | 2304 | OE1 | GLU | A | 322 | 66.875 | 48.605 | 27.546 | 1.00 | 30.00 |
| ATOM | 2305 | OE2 | GLU | A | 322 | 65.613 | 50.399 | 27.684 | 1.00 | 30.22 |
| ATOM | 2306 | C | GLU | A | 322 | 68.826 | 51.605 | 23.553 | 1.00 | 19.57 |
| ATOM | 2307 | O | GLU | A | 322 | 69.808 | 51.136 | 22.986 | 1.00 | 24.35 |
| ATOM | 2308 | N | TYR | A | 323 | 68.682 | 52.901 | 23.824 | 1.00 | 19.26 |
| ATOM | 2309 | CA | TYR | A | 323 | 69.698 | 53.893 | 23.464 | 1.00 | 19.31 |
| ATOM | 2310 | CB | TYR | A | 323 | 69.282 | 55.295 | 23.925 | 1.00 | 22.25 |
| ATOM | 2311 | CG | TYR | A | 323 | 68.382 | 56.044 | 22.971 | 1.00 | 22.48 |
| ATOM | 2312 | CD1 | TYR | A | 323 | 68.689 | 56.162 | 21.610 | 1.00 | 22.04 |
| ATOM | 2313 | CE1 | TYR | A | 323 | 67.847 | 56.859 | 20.750 | 1.00 | 21.95 |
| ATOM | 2314 | CD2 | TYR | A | 323 | 67.226 | 56.643 | 23.434 | 1.00 | 21.41 |
| ATOM | 2315 | CE2 | TYR | A | 323 | 66.386 | 57.337 | 22.587 | 1.00 | 24.39 |
| ATOM | 2316 | CZ | TYR | A | 323 | 66.698 | 57.442 | 21.244 | 1.00 | 23.89 |
| ATOM | 2317 | OH | TYR | A | 323 | 65.849 | 58.133 | 20.405 | 1.00 | 23.70 |
| ATOM | 2318 | C | TYR | A | 323 | 71.074 | 53.553 | 24.030 | 1.00 | 19.93 |
| ATOM | 2319 | O | TYR | A | 323 | 72.083 | 53.580 | 23.304 | 1.00 | 17.86 |
| ATOM | 2320 | N | THR | A | 324 | 71.127 | 53.228 | 25.339 | 1.00 | 19.13 |
| ATOM | 2321 | CA | THR | A | 324 | 72.401 | 52.875 | 25.971 | 1.00 | 15.51 |
| ATOM | 2322 | CB | THR | A | 324 | 72.339 | 52.986 | 27.507 | 1.00 | 17.08 |
| ATOM | 2323 | OG1 | THR | A | 324 | 71.807 | 54.245 | 27.879 | 1.00 | 17.91 |
| ATOM | 2324 | CG2 | THR | A | 324 | 73.699 | 52.853 | 28.147 | 1.00 | 11.22 |
| ATOM | 2325 | C | THR | A | 324 | 72.800 | 51.489 | 25.541 | 1.00 | 14.29 |
| ATOM | 2326 | O | THR | A | 324 | 72.045 | 50.542 | 25.741 | 1.00 | 14.20 |
| ATOM | 2327 | N | PRO | A | 325 | 73.977 | 51.373 | 24.886 | 1.00 | 16.36 |
| ATOM | 2328 | CD | PRO | A | 325 | 74.882 | 52.494 | 24.586 | 1.00 | 14.52 |
| ATOM | 2329 | CA | PRO | A | 325 | 74.486 | 50.099 | 24.329 | 1.00 | 14.39 |
| ATOM | 2330 | CB | PRO | A | 325 | 75.848 | 50.481 | 23.743 | 1.00 | 10.98 |
| ATOM | 2331 | CG | PRO | A | 325 | 75.763 | 51.937 | 23.498 | 1.00 | 15.41 |
| ATOM | 2332 | C | PRO | A | 325 | 74.636 | 48.983 | 25.365 | 1.00 | 15.34 |
| ATOM | 2333 | O | PRO | A | 325 | 74.214 | 47.840 | 25.130 | 1.00 | 12.17 |
| ATOM | 2334 | N | THR | A | 326 | 75.237 | 49.323 | 26.514 | 1.00 | 15.65 |
| ATOM | 2335 | CA | THR | A | 326 | 75.435 | 48.354 | 27.610 | 1.00 | 14.91 |
| ATOM | 2336 | CB | THR | A | 326 | 76.305 | 48.965 | 28.718 | 1.00 | 14.59 |
| ATOM | 2337 | OG1 | THR | A | 326 | 75.804 | 50.224 | 29.130 | 1.00 | 11.75 |
| ATOM | 2338 | CG2 | THR | A | 326 | 77.744 | 49.150 | 28.302 | 1.00 | 13.98 |
| ATOM | 2339 | C | THR | A | 326 | 74.119 | 47.875 | 28.196 | 1.00 | 14.19 |
| ATOM | 2340 | O | THR | A | 326 | 74.060 | 46.813 | 28.802 | 1.00 | 19.56 |
| ATOM | 2341 | N | ALA | A | 327 | 73.072 | 48.668 | 28.024 | 1.00 | 14.86 |
| ATOM | 2342 | CA | ALA | A | 327 | 71.744 | 48.358 | 28.542 | 1.00 | 15.10 |
| ATOM | 2343 | CB | ALA | A | 327 | 70.915 | 49.624 | 28.593 | 1.00 | 15.94 |
| ATOM | 2344 | C | ALA | A | 327 | 70.989 | 47.256 | 27.763 | 1.00 | 17.36 |
| ATOM | 2345 | O | ALA | A | 327 | 70.043 | 46.643 | 28.303 | 1.00 | 15.08 |
| ATOM | 2346 | N | ARG | A | 328 | 71.378 | 47.013 | 26.496 | 1.00 | 16.26 |
| ATOM | 2347 | CA | ARG | A | 328 | 70.705 | 45.993 | 25.656 | 1.00 | 13.02 |
| ATOM | 2348 | CB | ARG | A | 328 | 71.126 | 46.134 | 24.161 | 1.00 | 13.41 |
| ATOM | 2349 | CG | ARG | A | 328 | 70.732 | 47.450 | 23.514 | 1.00 | 14.63 |
| ATOM | 2350 | CD | ARG | A | 328 | 71.583 | 47.740 | 22.255 | 1.00 | 17.74 |
| ATOM | 2351 | NE | ARG | A | 328 | 71.432 | 49.141 | 21.822 | 1.00 | 14.76 |
| ATOM | 2352 | CZ | ARG | A | 328 | 72.398 | 49.869 | 21.296 | 1.00 | 10.22 |
| ATOM | 2353 | NH1 | ARG | A | 328 | 73.492 | 49.298 | 20.858 | 1.00 | 9.59 |
| ATOM | 2354 | NH2 | ARG | A | 328 | 72.237 | 51.187 | 21.166 | 1.00 | 8.94 |
| ATOM | 2355 | C | ARG | A | 328 | 71.057 | 44.586 | 26.096 | 1.00 | 6.14 |
| ATOM | 2356 | O | ARG | A | 328 | 72.166 | 44.329 | 26.486 | 1.00 | 8.50 |
| ATOM | 2357 | N | LEU | A | 329 | 70.133 | 43.656 | 25.949 | 1.00 | 8.48 |
| ATOM | 2358 | CA | LEU | A | 329 | 70.462 | 42.279 | 26.273 | 1.00 | 11.70 |
| ATOM | 2359 | CB | LEU | A | 329 | 69.274 | 41.342 | 26.034 | 1.00 | 13.47 |
| ATOM | 2360 | CG | LEU | A | 329 | 67.968 | 41.615 | 26.736 | 1.00 | 18.89 |
| ATOM | 2361 | CD1 | LEU | A | 329 | 66.876 | 40.769 | 26.102 | 1.00 | 18.93 |
| ATOM | 2362 | CD2 | LEU | A | 329 | 68.087 | 41.309 | 28.232 | 1.00 | 19.20 |
| ATOM | 2363 | C | LEU | A | 329 | 71.541 | 41.786 | 25.343 | 1.00 | 17.58 |
| ATOM | 2364 | O | LEU | A | 329 | 71.701 | 42.294 | 24.185 | 1.00 | 18.98 |
| ATOM | 2365 | N | THR | A | 330 | 72.224 | 40.737 | 25.793 | 1.00 | 17.60 |
| ATOM | 2366 | CA | THR | A | 330 | 73.209 | 40.089 | 24.989 | 1.00 | 19.09 |
| ATOM | 2367 | CB | THR | A | 330 | 74.271 | 39.413 | 25.846 | 1.00 | 19.08 |
| ATOM | 2368 | OG1 | THR | A | 330 | 73.702 | 38.399 | 26.639 | 1.00 | 20.66 |
| ATOM | 2369 | CG2 | THR | A | 330 | 75.081 | 40.347 | 26.725 | 1.00 | 22.70 |
| ATOM | 2370 | C | THR | A | 330 | 72.462 | 39.012 | 24.187 | 1.00 | 18.93 |
| ATOM | 2371 | O | THR | A | 330 | 71.310 | 38.670 | 24.495 | 1.00 | 19.21 |
| ATOM | 2372 | N | PRO | A | 331 | 73.073 | 38.459 | 23.155 | 1.00 | 17.26 |
| ATOM | 2373 | CD | PRO | A | 331 | 74.417 | 38.779 | 22.661 | 1.00 | 17.15 |
| ATOM | 2374 | CA | PRO | A | 331 | 72.409 | 37.423 | 22.365 | 1.00 | 18.01 |
| ATOM | 2375 | CB | PRO | A | 331 | 73.426 | 37.070 | 21.277 | 1.00 | 13.34 |
| ATOM | 2376 | CG | PRO | A | 331 | 74.381 | 38.211 | 21.255 | 1.00 | 17.27 |
| ATOM | 2377 | C | PRO | A | 331 | 72.060 | 36.207 | 23.250 | 1.00 | 16.55 |
| ATOM | 2378 | O | PRO | A | 331 | 70.967 | 35.670 | 23.168 | 1.00 | 19.08 |
| ATOM | 2379 | N | LEU | A | 332 | 72.976 | 35.786 | 24.112 | 1.00 | 18.64 |
| ATOM | 2380 | CA | LEU | A | 332 | 72.676 | 34.640 | 24.995 | 1.00 | 20.57 |
| ATOM | 2381 | CB | LEU | A | 332 | 73.895 | 34.158 | 25.770 | 1.00 | 22.27 |
| ATOM | 2382 | CG | LEU | A | 332 | 73.915 | 32.659 | 26.052 | 1.00 | 24.61 |
| ATOM | 2383 | CD1 | LEU | A | 332 | 73.755 | 31.869 | 24.756 | 1.00 | 26.96 |
| ATOM | 2384 | CD2 | LEU | A | 332 | 75.192 | 32.255 | 26.780 | 1.00 | 26.02 |
| ATOM | 2385 | C | LEU | A | 332 | 71.494 | 34.916 | 25.919 | 1.00 | 17.91 |
| ATOM | 2386 | O | LEU | A | 332 | 70.643 | 34.060 | 26.085 | 1.00 | 21.79 |
| ATOM | 2387 | N | GLU | A | 333 | 71.419 | 36.133 | 26.470 | 1.00 | 17.14 |
| ATOM | 2388 | CA | GLU | A | 333 | 70.303 | 36.544 | 27.335 | 1.00 | 17.62 |
| ATOM | 2389 | CB | GLU | A | 333 | 70.599 | 37.889 | 28.015 | 1.00 | 18.45 |
| ATOM | 2390 | CG | GLU | A | 333 | 71.668 | 37.822 | 29.095 | 1.00 | 19.81 |
| ATOM | 2391 | CD | GLU | A | 333 | 72.216 | 39.180 | 29.482 | 1.00 | 20.30 |
| ATOM | 2392 | OE1 | GLU | A | 333 | 73.258 | 39.216 | 30.163 | 1.00 | 25.89 |
| ATOM | 2393 | OE2 | GLU | A | 333 | 71.622 | 40.211 | 29.097 | 1.00 | 22.23 |
| ATOM | 2394 | C | GLU | A | 333 | 69.017 | 36.686 | 26.540 | 1.00 | 18.85 |
| ATOM | 2395 | O | GLU | A | 333 | 67.924 | 36.441 | 27.056 | 1.00 | 18.09 |
| ATOM | 2396 | N | ALA | A | 334 | 69.127 | 37.084 | 25.258 | 1.00 | 17.85 |
| ATOM | 2397 | CA | ALA | A | 334 | 67.917 | 37.214 | 24.449 | 1.00 | 16.32 |
| ATOM | 2398 | CB | ALA | A | 334 | 68.221 | 37.898 | 23.097 | 1.00 | 23.05 |
| ATOM | 2399 | C | ALA | A | 334 | 67.320 | 35.841 | 24.233 | 1.00 | 13.30 |
| ATOM | 2400 | O | ALA | A | 334 | 66.112 | 35.664 | 24.301 | 1.00 | 15.40 |
| ATOM | 2401 | N | CYS | A | 335 | 68.189 | 34.868 | 23.968 | 1.00 | 15.76 |
| ATOM | 2402 | CA | CYS | A | 335 | 67.786 | 33.476 | 23.728 | 1.00 | 18.48 |
| ATOM | 2403 | CB | CYS | A | 335 | 69.035 | 32.610 | 23.453 | 1.00 | 20.13 |
| ATOM | 2404 | SG | CYS | A | 335 | 69.650 | 32.566 | 21.730 | 1.00 | 23.99 |
| ATOM | 2405 | C | CYS | A | 335 | 67.038 | 32.884 | 24.945 | 1.00 | 20.44 |
| ATOM | 2406 | O | CYS | A | 335 | 66.181 | 32.014 | 24.790 | 1.00 | 20.18 |
| ATOM | 2407 | N | ALA | A | 336 | 67.407 | 33.338 | 26.156 | 1.00 | 21.45 |
| ATOM | 2408 | CA | ALA | A | 336 | 66.818 | 32.844 | 27.412 | 1.00 | 22.45 |
| ATOM | 2409 | CB | ALA | A | 336 | 67.845 | 32.902 | 28.554 | 1.00 | 24.12 |
| ATOM | 2410 | C | ALA | A | 336 | 65.546 | 33.594 | 27.780 | 1.00 | 21.53 |
| ATOM | 2411 | O | ALA | A | 336 | 64.811 | 33.174 | 28.670 | 1.00 | 24.07 |
| ATOM | 2412 | N | HIS | A | 337 | 65.278 | 34.687 | 27.078 | 1.00 | 20.73 |
| ATOM | 2413 | CA | HIS | A | 337 | 64.083 | 35.488 | 27.310 | 1.00 | 18.65 |
| ATOM | 2414 | CB | HIS | A | 337 | 64.014 | 36.651 | 26.301 | 1.00 | 15.94 |
| ATOM | 2415 | CG | HIS | A | 337 | 62.994 | 37.711 | 26.621 | 1.00 | 6.72 |
| ATOM | 2416 | CD2 | HIS | A | 337 | 63.128 | 38.941 | 27.167 | 1.00 | 7.32 |
| ATOM | 2417 | ND1 | HIS | A | 337 | 61.667 | 37.572 | 26.348 | 1.00 | 6.66 |
| ATOM | 2418 | CE1 | HIS | A | 337 | 61.015 | 38.662 | 26.701 | 1.00 | 5.00 |
| ATOM | 2419 | NE2 | HIS | A | 337 | 61.884 | 39.507 | 27.202 | 1.00 | 5.30 |
| ATOM | 2420 | C | HIS | A | 337 | 62.827 | 34.619 | 27.284 | 1.00 | 19.85 |
| ATOM | 2421 | O | HIS | A | 337 | 62.839 | 33.507 | 26.758 | 1.00 | 22.24 |
| ATOM | 2422 | N | SER | A | 338 | 61.756 | 35.104 | 27.886 | 1.00 | 19.23 |
| ATOM | 2423 | CA | SER | A | 338 | 60.510 | 34.350 | 27.961 | 1.00 | 21.39 |
| ATOM | 2424 | CB | SER | A | 338 | 59.595 | 34.938 | 29.048 | 1.00 | 21.54 |
| ATOM | 2425 | OG | SER | A | 338 | 58.610 | 35.797 | 28.503 | 1.00 | 26.05 |
| ATOM | 2426 | C | SER | A | 338 | 59.769 | 34.255 | 26.613 | 1.00 | 21.75 |
| ATOM | 2427 | O | SER | A | 338 | 58.915 | 33.383 | 26.425 | 1.00 | 19.44 |
| ATOM | 2428 | N | PHE | A | 339 | 60.093 | 35.154 | 25.681 | 1.00 | 24.18 |
| ATOM | 2429 | CA | PHE | A | 339 | 59.444 | 35.141 | 24.371 | 1.00 | 24.18 |
| ATOM | 2430 | CB | PHE | A | 339 | 59.962 | 36.276 | 23.467 | 1.00 | 24.91 |
| ATOM | 2431 | CG | PHE | A | 339 | 59.313 | 36.275 | 22.100 | 1.00 | 26.05 |
| ATOM | 2432 | CD1 | PHE | A | 339 | 60.085 | 36.296 | 20.943 | 1.00 | 25.96 |
| ATOM | 2433 | CD2 | PHE | A | 339 | 57.934 | 36.251 | 21.977 | 1.00 | 25.69 |
| ATOM | 2434 | CE1 | PHE | A | 339 | 59.489 | 36.301 | 19.696 | 1.00 | 24.85 |
| ATOM | 2435 | CE2 | PHE | A | 339 | 57.330 | 36.253 | 20.730 | 1.00 | 25.53 |
| ATOM | 2436 | CZ | PHE | A | 339 | 58.112 | 36.281 | 19.590 | 1.00 | 23.89 |
| ATOM | 2437 | C | PHE | A | 339 | 59.675 | 33.805 | 23.693 | 1.00 | 22.02 |
| ATOM | 2438 | O | PHE | A | 339 | 58.812 | 33.305 | 22.995 | 1.00 | 22.73 |
| ATOM | 2439 | N | PHE | A | 340 | 60.853 | 33.242 | 23.904 | 1.00 | 22.97 |
| ATOM | 2440 | CA | PHE | A | 340 | 61.213 | 31.964 | 23.299 | 1.00 | 23.44 |
| ATOM | 2441 | CB | PHE | A | 340 | 62.696 | 31.959 | 22.954 | 1.00 | 21.17 |
| ATOM | 2442 | CG | PHE | A | 340 | 63.108 | 33.128 | 22.105 | 1.00 | 18.96 |
| ATOM | 2443 | CD1 | PHE | A | 340 | 62.623 | 33.269 | 20.811 | 1.00 | 15.28 |
| ATOM | 2444 | CD2 | PHE | A | 340 | 63.983 | 34.082 | 22.598 | 1.00 | 16.96 |
| ATOM | 2445 | CE1 | PHE | A | 340 | 63.013 | 34.338 | 20.034 | 1.00 | 16.19 |
| ATOM | 2446 | CE2 | PHE | A | 340 | 64.375 | 35.156 | 21.825 | 1.00 | 16.66 |
| ATOM | 2447 | CZ | PHE | A | 340 | 63.886 | 35.282 | 20.534 | 1.00 | 17.00 |
| ATOM | 2448 | C | PHE | A | 340 | 60.855 | 30.741 | 24.156 | 1.00 | 26.22 |
| ATOM | 2449 | O | PHE | A | 340 | 61.224 | 29.616 | 23.806 | 1.00 | 26.38 |
| ATOM | 2450 | N | ASP | A | 341 | 60.136 | 30.952 | 25.272 | 1.00 | 28.20 |
| ATOM | 2451 | CA | ASP | A | 341 | 59.744 | 29.840 | 26.150 | 1.00 | 27.65 |
| ATOM | 2452 | CB | ASP | A | 341 | 58.830 | 30.323 | 27.290 | 1.00 | 28.09 |
| ATOM | 2453 | CG | ASP | A | 341 | 59.562 | 31.111 | 28.373 | 1.00 | 28.12 |
| ATOM | 2454 | OD1 | ASP | A | 341 | 60.796 | 30.975 | 28.498 | 1.00 | 27.75 |
| ATOM | 2455 | OD2 | ASP | A | 341 | 58.892 | 31.868 | 29.098 | 1.00 | 30.69 |
| ATOM | 2456 | C | ASP | A | 341 | 59.033 | 28.755 | 25.367 | 1.00 | 28.02 |
| ATOM | 2457 | O | ASP | A | 341 | 59.270 | 27.565 | 25.583 | 1.00 | 29.18 |
| ATOM | 2458 | N | GLU | A | 342 | 58.156 | 29.163 | 24.451 | 1.00 | 28.63 |
| ATOM | 2459 | CA | GLU | A | 342 | 57.415 | 28.198 | 23.640 | 1.00 | 27.71 |
| ATOM | 2460 | CB | GLU | A | 342 | 56.547 | 28.896 | 22.599 | 1.00 | 28.66 |
| ATOM | 2461 | CG | GLU | A | 342 | 55.801 | 27.939 | 21.678 | 1.00 | 29.56 |
| ATOM | 2462 | CD | GLU | A | 342 | 54.648 | 28.595 | 20.949 | 1.00 | 30.24 |
| ATOM | 2463 | OE1 | GLU | A | 342 | 53.632 | 27.909 | 20.714 | 1.00 | 33.64 |
| ATOM | 2464 | OE2 | GLU | A | 342 | 54.756 | 29.794 | 20.612 | 1.00 | 31.91 |
| ATOM | 2465 | C | GLU | A | 342 | 58.352 | 27.192 | 22.984 | 1.00 | 27.16 |
| ATOM | 2466 | O | GLU | A | 342 | 58.122 | 25.997 | 23.054 | 1.00 | 28.46 |
| ATOM | 2467 | N | LEU | A | 343 | 59.422 | 27.681 | 22.363 | 1.00 | 27.00 |
| ATOM | 2468 | CA | LEU | A | 343 | 60.393 | 26.802 | 21.718 | 1.00 | 26.58 |
| ATOM | 2469 | CB | LEU | A | 343 | 61.505 | 27.629 | 21.060 | 1.00 | 25.90 |
| ATOM | 2470 | CG | LEU | A | 343 | 61.058 | 28.712 | 20.058 | 1.00 | 26.74 |
| ATOM | 2471 | CD1 | LEU | A | 343 | 62.260 | 29.339 | 19.374 | 1.00 | 23.52 |
| ATOM | 2472 | CD2 | LEU | A | 343 | 60.089 | 28.140 | 19.028 | 1.00 | 22.74 |
| ATOM | 2473 | C | LEU | A | 343 | 60.999 | 25.806 | 22.715 | 1.00 | 28.14 |
| ATOM | 2474 | O | LEU | A | 343 | 61.486 | 24.744 | 22.326 | 1.00 | 28.82 |
| ATOM | 2475 | N | ARG | A | 344 | 60.981 | 26.173 | 23.998 | 1.00 | 29.57 |
| ATOM | 2476 | CA | ARG | A | 344 | 61.538 | 25.353 | 25.070 | 1.00 | 30.15 |
| ATOM | 2477 | CB | ARG | A | 344 | 61.884 | 26.262 | 26.256 | 1.00 | 29.53 |
| ATOM | 2478 | CG | ARG | A | 344 | 63.311 | 26.106 | 26.754 | 1.00 | 25.45 |
| ATOM | 2479 | CD | ARG | A | 344 | 64.113 | 27.395 | 26.664 | 1.00 | 21.49 |
| ATOM | 2480 | NE | ARG | A | 344 | 63.314 | 28.608 | 26.901 | 1.00 | 18.01 |
| ATOM | 2481 | CZ | ARG | A | 344 | 63.742 | 29.830 | 26.603 | 1.00 | 13.11 |
| ATOM | 2482 | NH1 | ARG | A | 344 | 64.917 | 29.999 | 26.036 | 1.00 | 15.16 |
| ATOM | 2483 | NH2 | ARG | A | 344 | 63.002 | 30.887 | 26.875 | 1.00 | 16.28 |
| ATOM | 2484 | C | ARG | A | 344 | 60.571 | 24.249 | 25.515 | 1.00 | 31.98 |
| ATOM | 2485 | O | ARG | A | 344 | 60.982 | 23.281 | 26.150 | 1.00 | 33.79 |
| ATOM | 2486 | N | ASP | A | 345 | 59.293 | 24.406 | 25.180 | 1.00 | 33.25 |
| ATOM | 2487 | CA | ASP | A | 345 | 58.242 | 23.449 | 25.530 | 1.00 | 35.71 |
| ATOM | 2488 | CB | ASP | A | 345 | 56.883 | 24.041 | 25.124 | 1.00 | 36.83 |
| ATOM | 2489 | CG | ASP | A | 345 | 55.660 | 23.265 | 25.591 | 1.00 | 37.61 |
| ATOM | 2490 | OD1 | ASP | A | 345 | 54.573 | 23.873 | 25.653 | 1.00 | 39.11 |
| ATOM | 2491 | OD2 | ASP | A | 345 | 55.774 | 22.056 | 25.871 | 1.00 | 39.24 |
| ATOM | 2492 | C | ASP | A | 345 | 58.463 | 22.077 | 24.863 | 1.00 | 37.75 |
| ATOM | 2493 | O | ASP | A | 345 | 58.633 | 21.987 | 23.647 | 1.00 | 37.67 |
| ATOM | 2494 | N | PRO | A | 346 | 58.451 | 20.981 | 25.662 | 1.00 | 38.99 |
| ATOM | 2495 | CD | PRO | A | 346 | 58.238 | 20.989 | 27.122 | 1.00 | 38.44 |
| ATOM | 2496 | CA | PRO | A | 346 | 58.638 | 19.614 | 25.145 | 1.00 | 39.68 |
| ATOM | 2497 | CB | PRO | A | 346 | 58.533 | 18.726 | 26.396 | 1.00 | 39.31 |
| ATOM | 2498 | CG | PRO | A | 346 | 58.747 | 19.646 | 27.551 | 1.00 | 38.56 |
| ATOM | 2499 | C | PRO | A | 346 | 57.539 | 19.243 | 24.155 | 1.00 | 40.56 |
| ATOM | 2500 | O | PRO | A | 346 | 57.758 | 18.461 | 23.228 | 1.00 | 40.27 |
| ATOM | 2501 | N | ASN | A | 347 | 56.358 | 19.760 | 24.424 | 1.00 | 42.43 |
| ATOM | 2502 | CA | ASN | A | 347 | 55.205 | 19.522 | 23.560 | 1.00 | 44.15 |
| ATOM | 2503 | CB | ASN | A | 347 | 53.928 | 19.386 | 24.364 | 1.00 | 45.69 |
| ATOM | 2504 | CG | ASN | A | 347 | 54.183 | 18.874 | 25.759 | 1.00 | 46.82 |
| ATOM | 2505 | OD1 | ASN | A | 347 | 54.107 | 19.621 | 26.717 | 1.00 | 48.40 |
| ATOM | 2506 | ND2 | ASN | A | 347 | 54.478 | 17.585 | 25.876 | 1.00 | 46.44 |
| ATOM | 2507 | C | ASN | A | 347 | 55.101 | 20.648 | 22.557 | 1.00 | 45.30 |
| ATOM | 2508 | O | ASN | A | 347 | 54.367 | 21.603 | 22.772 | 1.00 | 45.75 |
| ATOM | 2509 | N | VAL | A | 348 | 55.800 | 20.568 | 21.428 | 1.00 | 46.70 |
| ATOM | 2510 | CA | VAL | A | 348 | 55.825 | 21.669 | 20.449 | 1.00 | 46.47 |
| ATOM | 2511 | CB | VAL | A | 348 | 57.250 | 22.264 | 20.391 | 1.00 | 46.04 |
| ATOM | 2512 | CG1 | VAL | A | 348 | 57.185 | 23.712 | 19.955 | 1.00 | 44.04 |
| ATOM | 2513 | CG2 | VAL | A | 348 | 57.971 | 22.124 | 21.725 | 1.00 | 44.33 |
| ATOM | 2514 | C | VAL | A | 348 | 55.537 | 21.252 | 19.033 | 1.00 | 47.56 |
| ATOM | 2515 | O | VAL | A | 348 | 54.408 | 21.144 | 18.578 | 1.00 | 49.67 |
| ATOM | 2516 | N | LYS | A | 349 | 56.733 | 21.027 | 18.372 | 1.00 | 49.07 |
| ATOM | 2517 | CA | LYS | A | 349 | 56.978 | 20.595 | 16.967 | 1.00 | 49.74 |
| ATOM | 2518 | CB | LYS | A | 349 | 56.741 | 19.087 | 16.859 | 1.00 | 51.67 |
| ATOM | 2519 | CG | LYS | A | 349 | 55.344 | 18.739 | 17.305 | 1.00 | 53.90 |
| ATOM | 2520 | CD | LYS | A | 349 | 54.809 | 17.547 | 16.564 | 1.00 | 56.03 |
| ATOM | 2521 | CE | LYS | A | 349 | 54.853 | 16.307 | 17.446 | 1.00 | 58.27 |
| ATOM | 2522 | NZ | LYS | A | 349 | 54.174 | 15.146 | 16.799 | 1.00 | 61.28 |
| ATOM | 2523 | C | LYS | A | 349 | 56.022 | 21.275 | 16.040 | 1.00 | 48.95 |
| ATOM | 2524 | O | LYS | A | 349 | 54.979 | 20.700 | 15.716 | 1.00 | 48.99 |
| ATOM | 2525 | N | LED | A | 350 | 56.327 | 22.484 | 15.565 | 1.00 | 47.74 |
| ATOM | 2526 | CA | LED | A | 350 | 55.066 | 23.026 | 15.181 | 1.00 | 46.51 |
| ATOM | 2527 | CB | LED | A | 350 | 54.856 | 24.259 | 16.052 | 1.00 | 46.83 |
| ATOM | 2528 | CG | LED | A | 350 | 53.560 | 24.260 | 16.887 | 1.00 | 46.94 |
| ATOM | 2529 | CD1 | LED | A | 350 | 53.841 | 24.613 | 18.334 | 1.00 | 47.80 |
| ATOM | 2530 | CD2 | LED | A | 350 | 52.555 | 25.234 | 16.283 | 1.00 | 47.66 |
| ATOM | 2531 | C | LED | A | 350 | 54.460 | 23.377 | 13.853 | 1.00 | 45.92 |
| ATOM | 2532 | O | LED | A | 350 | 53.647 | 22.581 | 13.380 | 1.00 | 46.10 |
| ATOM | 2533 | N | PRO | A | 351 | 54.759 | 24.499 | 13.169 | 1.00 | 45.11 |
| ATOM | 2534 | CD | PRO | A | 351 | 55.423 | 25.732 | 13.632 | 1.00 | 44.07 |
| ATOM | 2535 | CA | PRO | A | 351 | 53.722 | 24.809 | 12.154 | 1.00 | 44.28 |
| ATOM | 2536 | CB | PRO | A | 351 | 54.397 | 25.950 | 11.411 | 1.00 | 42.95 |
| ATOM | 2537 | CG | PRO | A | 351 | 55.110 | 26.705 | 12.512 | 1.00 | 43.47 |
| ATOM | 2538 | C | PRO | A | 351 | 53.060 | 23.662 | 11.434 | 1.00 | 43.92 |
| ATOM | 2539 | O | PRO | A | 351 | 51.833 | 23.548 | 11.439 | 1.00 | 45.39 |
| ATOM | 2540 | N | ASN | A | 352 | 53.870 | 22.805 | 10.828 | 1.00 | 44.49 |
| ATOM | 2541 | CA | ASN | A | 352 | 53.312 | 21.722 | 10.058 | 1.00 | 45.59 |
| ATOM | 2542 | CB | ASN | A | 352 | 53.931 | 21.727 | 8.659 | 1.00 | 45.84 |
| ATOM | 2543 | CG | ASN | A | 352 | 53.124 | 22.481 | 7.640 | 1.00 | 48.02 |
| ATOM | 2544 | OD1 | ASN | A | 352 | 53.320 | 22.301 | 6.443 | 1.00 | 49.81 |
| ATOM | 2545 | ND2 | ASN | A | 352 | 52.225 | 23.327 | 8.109 | 1.00 | 47.62 |
| ATOM | 2546 | C | ASN | A | 352 | 53.596 | 20.357 | 10.683 | 1.00 | 46.48 |
| ATOM | 2547 | O | ASN | A | 352 | 53.398 | 20.154 | 11.889 | 1.00 | 46.98 |
| ATOM | 2548 | N | GLY | A | 353 | 54.074 | 19.429 | 9.862 | 1.00 | 45.86 |
| ATOM | 2549 | CA | GLY | A | 353 | 54.395 | 18.106 | 10.351 | 1.00 | 47.10 |
| ATOM | 2550 | C | GLY | A | 353 | 55.887 | 17.913 | 10.413 | 1.00 | 47.56 |
| ATOM | 2551 | O | GLY | A | 353 | 56.418 | 16.955 | 9.851 | 1.00 | 48.69 |
| ATOM | 2552 | N | ARG | A | 354 | 56.567 | 18.848 | 11.074 | 1.00 | 47.79 |
| ATOM | 2553 | CA | ARG | A | 354 | 58.015 | 18.803 | 11.181 | 1.00 | 48.26 |
| ATOM | 2554 | CB | ARG | A | 354 | 58.631 | 19.909 | 10.318 | 1.00 | 50.37 |
| ATOM | 2555 | CG | ARG | A | 354 | 58.796 | 19.527 | 8.849 | 1.00 | 53.05 |
| ATOM | 2556 | CD | ARG | A | 354 | 57.593 | 19.956 | 8.003 | 1.00 | 55.20 |
| ATOM | 2557 | NE | ARG | A | 354 | 57.294 | 21.393 | 8.135 | 1.00 | 56.34 |
| ATOM | 2558 | CZ | ARG | A | 354 | 57.192 | 22.256 | 7.105 | 1.00 | 57.36 |
| ATOM | 2559 | NH1 | ARG | A | 354 | 57.322 | 21.841 | 5.845 | 1.00 | 57.10 |
| ATOM | 2560 | NH2 | ARG | A | 354 | 56.944 | 23.547 | 7.341 | 1.00 | 57.28 |
| ATOM | 2561 | C | ARG | A | 354 | 58.480 | 18.961 | 12.622 | 1.00 | 47.05 |
| ATOM | 2562 | O | ARG | A | 354 | 57.901 | 19.724 | 13.396 | 1.00 | 46.38 |
| ATOM | 2563 | N | ASP | A | 355 | 59.547 | 18.244 | 12.964 | 1.00 | 45.55 |
| ATOM | 2564 | CA | ASP | A | 355 | 60.110 | 18.318 | 14.298 | 1.00 | 43.83 |
| ATOM | 2565 | CB | ASP | A | 355 | 61.035 | 17.126 | 14.621 | 1.00 | 44.55 |
| ATOM | 2566 | CG | ASP | A | 355 | 61.108 | 16.051 | 13.547 | 1.00 | 44.92 |
| ATOM | 2567 | OD1 | ASP | A | 355 | 60.108 | 15.328 | 13.356 | 1.00 | 43.70 |
| ATOM | 2568 | OD2 | ASP | A | 355 | 62.178 | 15.920 | 12.909 | 1.00 | 46.40 |
| ATOM | 2569 | C | ASP | A | 355 | 60.879 | 19.619 | 14.459 | 1.00 | 43.16 |
| ATOM | 2570 | O | ASP | A | 355 | 61.408 | 20.168 | 13.487 | 1.00 | 43.03 |
| ATOM | 2571 | N | THR | A | 356 | 60.945 | 20.101 | 15.689 | 1.00 | 40.81 |
| ATOM | 2572 | CA | THR | A | 356 | 61.661 | 21.326 | 15.987 | 1.00 | 38.24 |
| ATOM | 2573 | CB | THR | A | 356 | 61.513 | 21.676 | 17.480 | 1.00 | 38.43 |
| ATOM | 2574 | OG1 | THR | A | 356 | 62.687 | 22.274 | 18.002 | 1.00 | 37.98 |
| ATOM | 2575 | CG2 | THR | A | 356 | 61.161 | 20.496 | 18.364 | 1.00 | 37.12 |
| ATOM | 2576 | C | THR | A | 356 | 63.135 | 21.167 | 15.607 | 1.00 | 37.28 |
| ATOM | 2577 | O | THR | A | 356 | 63.755 | 20.154 | 15.932 | 1.00 | 36.68 |
| ATOM | 2578 | N | PRO | A | 357 | 63.715 | 22.160 | 14.901 | 1.00 | 35.88 |
| ATOM | 2579 | CD | PRO | A | 357 | 63.051 | 23.389 | 14.448 | 1.00 | 34.92 |
| ATOM | 2580 | CA | PRO | A | 357 | 65.119 | 22.109 | 14.488 | 1.00 | 35.32 |
| ATOM | 2581 | CB | PRO | A | 357 | 65.376 | 23.464 | 13.818 | 1.00 | 35.57 |
| ATOM | 2582 | CG | PRO | A | 357 | 64.201 | 24.316 | 14.172 | 1.00 | 35.66 |
| ATOM | 2583 | C | PRO | A | 357 | 66.043 | 21.909 | 15.684 | 1.00 | 36.40 |
| ATOM | 2584 | O | PRO | A | 357 | 65.604 | 21.957 | 16.836 | 1.00 | 36.57 |
| ATOM | 2585 | N | ALA | A | 358 | 67.311 | 21.653 | 15.401 | 1.00 | 36.04 |
| ATOM | 2586 | CA | ALA | A | 358 | 68.308 | 21.399 | 16.430 | 1.00 | 36.22 |
| ATOM | 2587 | CB | ALA | A | 358 | 69.566 | 20.801 | 15.822 | 1.00 | 37.16 |
| ATOM | 2588 | C | ALA | A | 358 | 68.647 | 22.610 | 17.308 | 1.00 | 36.38 |
| ATOM | 2589 | O | ALA | A | 358 | 69.722 | 23.196 | 17.186 | 1.00 | 37.29 |
| ATOM | 2590 | N | LEU | A | 359 | 67.758 | 22.931 | 18.240 | 1.00 | 35.60 |
| ATOM | 2591 | CA | LEU | A | 359 | 68.011 | 24.004 | 19.186 | 1.00 | 35.86 |
| ATOM | 2592 | CB | LEU | A | 359 | 66.750 | 24.827 | 19.489 | 1.00 | 34.90 |
| ATOM | 2593 | CG | LEU | A | 359 | 65.744 | 25.034 | 18.349 | 1.00 | 35.74 |
| ATOM | 2594 | CD1 | LEU | A | 359 | 64.380 | 25.401 | 18.924 | 1.00 | 36.49 |
| ATOM | 2595 | CD2 | LEU | A | 359 | 66.220 | 26.108 | 17.378 | 1.00 | 33.17 |
| ATOM | 2596 | C | LEU | A | 359 | 68.539 | 23.355 | 20.470 | 1.00 | 36.74 |
| ATOM | 2597 | O | LEU | A | 359 | 68.464 | 22.121 | 20.625 | 1.00 | 38.67 |
| ATOM | 2598 | N | PHE | A | 360 | 69.073 | 24.154 | 21.384 | 1.00 | 35.31 |
| ATOM | 2599 | CA | PHE | A | 360 | 69.604 | 23.599 | 22.639 | 1.00 | 33.68 |
| ATOM | 2600 | CB | PHE | A | 360 | 68.592 | 22.645 | 23.297 | 1.00 | 29.33 |
| ATOM | 2601 | CG | PHE | A | 360 | 67.151 | 23.028 | 23.092 | 1.00 | 25.75 |
| ATOM | 2602 | CD1 | PHE | A | 360 | 66.215 | 22.074 | 22.745 | 1.00 | 24.32 |
| ATOM | 2603 | CD2 | PHE | A | 360 | 66.737 | 24.340 | 23.245 | 1.00 | 23.11 |
| ATOM | 2604 | CE1 | PHE | A | 360 | 64.893 | 22.416 | 22.557 | 1.00 | 22.23 |
| ATOM | 2605 | CE2 | PHE | A | 360 | 65.415 | 24.688 | 23.058 | 1.00 | 21.69 |
| ATOM | 2606 | CZ | PHE | A | 360 | 64.493 | 23.723 | 22.714 | 1.00 | 22.31 |
| ATOM | 2607 | C | PHE | A | 360 | 70.938 | 22.890 | 22.401 | 1.00 | 34.08 |
| ATOM | 2608 | O | PHE | A | 360 | 71.581 | 22.413 | 23.338 | 1.00 | 34.63 |
| ATOM | 2609 | N | ASN | A | 361 | 71.348 | 22.863 | 21.132 | 1.00 | 35.29 |
| ATOM | 2610 | CA | ASN | A | 361 | 72.609 | 22.265 | 20.670 | 1.00 | 35.58 |
| ATOM | 2611 | CB | ASN | A | 361 | 72.640 | 22.420 | 19.139 | 1.00 | 36.74 |
| ATOM | 2612 | CG | ASN | A | 361 | 73.165 | 21.225 | 18.392 | 1.00 | 38.91 |
| ATOM | 2613 | OD1 | ASN | A | 361 | 73.503 | 20.205 | 18.986 | 1.00 | 42.48 |
| ATOM | 2614 | ND2 | ASN | A | 361 | 73.235 | 21.347 | 17.069 | 1.00 | 39.99 |
| ATOM | 2615 | C | ASN | A | 361 | 73.830 | 23.005 | 21.272 | 1.00 | 34.58 |
| ATOM | 2616 | O | ASN | A | 361 | 74.969 | 22.786 | 20.853 | 1.00 | 33.31 |
| ATOM | 2617 | N | PHE | A | 362 | 73.567 | 23.870 | 22.265 | 1.00 | 33.34 |
| ATOM | 2618 | CA | PHE | A | 362 | 74.595 | 24.720 | 22.895 | 1.00 | 30.24 |
| ATOM | 2619 | CB | PHE | A | 362 | 74.044 | 25.448 | 24.128 | 1.00 | 26.30 |
| ATOM | 2620 | CG | PHE | A | 362 | 73.059 | 26.527 | 23.796 | 1.00 | 24.99 |
| ATOM | 2621 | CD1 | PHE | A | 362 | 71.710 | 26.344 | 24.031 | 1.00 | 24.49 |
| ATOM | 2622 | CD2 | PHE | A | 362 | 73.480 | 27.724 | 23.233 | 1.00 | 24.20 |
| ATOM | 2623 | CE1 | PHE | A | 362 | 70.797 | 27.336 | 23.704 | 1.00 | 25.12 |
| ATOM | 2624 | CE2 | PHE | A | 362 | 72.571 | 28.716 | 22.909 | 1.00 | 22.66 |
| ATOM | 2625 | CZ | PHE | A | 362 | 71.231 | 28.521 | 23.141 | 1.00 | 21.69 |
| ATOM | 2626 | C | PHE | A | 362 | 75.863 | 23.960 | 23.252 | 1.00 | 29.70 |
| ATOM | 2627 | O | PHE | A | 362 | 75.833 | 22.787 | 23.624 | 1.00 | 31.38 |
| ATOM | 2628 | N | THR | A | 363 | 76.973 | 24.666 | 23.115 | 1.00 | 28.72 |
| ATOM | 2629 | CA | THR | A | 363 | 78.283 | 24.139 | 23.426 | 1.00 | 30.32 |
| ATOM | 2630 | CB | THR | A | 363 | 79.118 | 23.949 | 22.160 | 1.00 | 30.33 |
| ATOM | 2631 | OG1 | THR | A | 363 | 79.626 | 25.227 | 21.730 | 1.00 | 29.69 |
| ATOM | 2632 | CG2 | THR | A | 363 | 78.264 | 23.324 | 21.073 | 1.00 | 31.54 |
| ATOM | 2633 | C | THR | A | 363 | 78.993 | 25.132 | 24.285 | 1.00 | 29.90 |
| ATOM | 2634 | O | THR | A | 363 | 78.570 | 26.284 | 24.450 | 1.00 | 31.27 |
| ATOM | 2635 | N | THR | A | 364 | 80.157 | 24.708 | 24.797 | 1.00 | 31.32 |
| ATOM | 2636 | CA | THR | A | 364 | 80.885 | 25.620 | 25.708 | 1.00 | 31.31 |
| ATOM | 2637 | CB | THR | A | 364 | 82.183 | 25.077 | 26.188 | 1.00 | 31.99 |
| ATOM | 2638 | OG1 | THR | A | 364 | 81.967 | 23.911 | 26.995 | 1.00 | 33.21 |
| ATOM | 2639 | CG2 | THR | A | 364 | 82.909 | 26.131 | 26.996 | 1.00 | 32.54 |
| ATOM | 2640 | C | THR | A | 364 | 81.244 | 26.991 | 25.221 | 1.00 | 31.22 |
| ATOM | 2641 | O | THR | A | 364 | 80.931 | 27.967 | 25.917 | 1.00 | 32.20 |
| ATOM | 2642 | N | GLN | A | 365 | 81.864 | 27.183 | 24.096 | 1.00 | 31.18 |
| ATOM | 2643 | CA | GLN | A | 365 | 82.097 | 28.555 | 23.672 | 1.00 | 29.69 |
| ATOM | 2644 | CB | GLN | A | 365 | 82.591 | 28.657 | 22.225 | 1.00 | 31.29 |
| ATOM | 2645 | CG | GLN | A | 365 | 84.066 | 28.448 | 22.048 | 1.00 | 31.31 |
| ATOM | 2646 | CD | GLN | A | 365 | 84.834 | 29.709 | 21.698 | 1.00 | 28.08 |
| ATOM | 2647 | OE1 | GLN | A | 365 | 84.239 | 30.773 | 21.479 | 1.00 | 27.43 |
| ATOM | 2648 | NE2 | GLN | A | 365 | 86.156 | 29.819 | 21.590 | 1.00 | 32.84 |
| ATOM | 2649 | C | GLN | A | 365 | 80.809 | 29.336 | 23.556 | 1.00 | 27.35 |
| ATOM | 2650 | O | GLN | A | 365 | 80.758 | 30.523 | 23.902 | 1.00 | 27.88 |
| ATOM | 2651 | N | GLU | A | 366 | 79.767 | 28.699 | 23.081 | 1.00 | 25.28 |
| ATOM | 2652 | CA | GLU | A | 366 | 78.560 | 29.472 | 22.934 | 1.00 | 26.02 |
| ATOM | 2653 | CB | GLU | A | 366 | 77.497 | 28.614 | 22.291 | 1.00 | 24.56 |
| ATOM | 2654 | CG | GLU | A | 366 | 77.570 | 28.538 | 20.791 | 1.00 | 23.39 |
| ATOM | 2655 | CD | GLU | A | 366 | 76.295 | 27.905 | 20.307 | 1.00 | 20.84 |
| ATOM | 2656 | OE1 | GLU | A | 366 | 75.379 | 28.656 | 19.899 | 1.00 | 18.16 |
| ATOM | 2657 | OE2 | GLU | A | 366 | 76.200 | 26.667 | 20.333 | 1.00 | 21.04 |
| ATOM | 2658 | C | GLU | A | 366 | 78.018 | 30.001 | 24.257 | 1.00 | 25.56 |
| ATOM | 2659 | O | GLU | A | 366 | 77.440 | 31.086 | 24.316 | 1.00 | 26.33 |
| ATOM | 2660 | N | LEU | A | 367 | 78.235 | 29.243 | 25.311 | 1.00 | 27.66 |
| ATOM | 2661 | CA | LEU | A | 367 | 77.747 | 29.637 | 26.617 | 1.00 | 27.22 |
| ATOM | 2662 | CB | LEU | A | 367 | 77.389 | 28.409 | 27.463 | 1.00 | 23.10 |
| ATOM | 2663 | CG | LEU | A | 367 | 76.197 | 27.579 | 26.998 | 1.00 | 22.63 |
| ATOM | 2664 | CD1 | LEU | A | 367 | 76.279 | 26.163 | 27.553 | 1.00 | 24.80 |
| ATOM | 2665 | CD2 | LEU | A | 367 | 74.886 | 28.228 | 27.395 | 1.00 | 22.27 |
| ATOM | 2666 | C | LEU | A | 367 | 78.782 | 30.491 | 27.342 | 1.00 | 27.79 |
| ATOM | 2667 | O | LEU | A | 367 | 78.474 | 31.114 | 28.368 | 1.00 | 29.77 |
| ATOM | 2668 | N | SER | A | 368 | 80.017 | 30.518 | 26.796 | 1.00 | 28.63 |
| ATOM | 2669 | CA | SER | A | 368 | 81.163 | 31.252 | 27.355 | 1.00 | 30.09 |
| ATOM | 2670 | CB | SER | A | 368 | 82.311 | 31.408 | 26.353 | 1.00 | 30.06 |
| ATOM | 2671 | OG | SER | A | 368 | 82.146 | 32.543 | 25.524 | 1.00 | 32.28 |
| ATOM | 2672 | C | SER | A | 368 | 80.794 | 32.563 | 28.040 | 1.00 | 31.79 |
| ATOM | 2673 | O | SER | A | 368 | 81.462 | 32.988 | 28.998 | 1.00 | 32.23 |
| ATOM | 2674 | N | SER | A | 369 | 79.711 | 33.174 | 27.586 | 1.00 | 31.30 |
| ATOM | 2675 | CA | SER | A | 369 | 79.225 | 34.393 | 28.200 | 1.00 | 31.27 |
| ATOM | 2676 | CB | SER | A | 369 | 78.832 | 35.437 | 27.141 | 1.00 | 28.64 |
| ATOM | 2677 | OG | SER | A | 369 | 77.432 | 35.662 | 27.104 | 1.00 | 23.51 |
| ATOM | 2678 | C | SER | A | 369 | 78.040 | 34.011 | 29.084 | 1.00 | 33.13 |
| ATOM | 2679 | O | SER | A | 369 | 76.968 | 33.649 | 28.587 | 1.00 | 37.45 |
| ATOM | 2680 | N | ASN | A | 370 | 78.237 | 34.040 | 30.384 | 1.00 | 31.36 |
| ATOM | 2681 | CA | ASN | A | 370 | 77.172 | 33.659 | 31.306 | 1.00 | 29.84 |
| ATOM | 2682 | CB | ASN | A | 370 | 75.917 | 34.511 | 31.061 | 1.00 | 31.79 |
| ATOM | 2683 | CG | ASN | A | 370 | 75.177 | 34.854 | 32.344 | 1.00 | 32.69 |
| ATOM | 2684 | OD1 | ASN | A | 370 | 74.323 | 35.750 | 32.368 | 1.00 | 33.79 |
| ATOM | 2685 | ND2 | ASN | A | 370 | 75.498 | 34.140 | 33.414 | 1.00 | 30.98 |
| ATOM | 2686 | C | ASN | A | 370 | 76.835 | 32.155 | 31.241 | 1.00 | 26.34 |
| ATOM | 2687 | O | ASN | A | 370 | 75.697 | 31.774 | 30.963 | 1.00 | 27.40 |
| ATOM | 2688 | N | PRO | A | 371 | 77.815 | 31.278 | 31.516 | 1.00 | 24.52 |
| ATOM | 2689 | CD | PRO | A | 371 | 79.202 | 31.636 | 31.868 | 1.00 | 24.13 |
| ATOM | 2690 | CA | PRO | A | 371 | 77.611 | 29.816 | 31.509 | 1.00 | 25.51 |
| ATOM | 2691 | CB | PRO | A | 371 | 78.909 | 29.272 | 32.100 | 1.00 | 25.50 |
| ATOM | 2692 | CG | PRO | A | 371 | 79.923 | 30.323 | 31.797 | 1.00 | 24.03 |
| ATOM | 2693 | C | PRO | A | 371 | 76.371 | 29.337 | 32.309 | 1.00 | 26.66 |
| ATOM | 2694 | O | PRO | A | 371 | 75.691 | 28.393 | 31.886 | 1.00 | 27.02 |
| ATOM | 2695 | N | PRO | A | 372 | 76.052 | 29.960 | 33.478 | 1.00 | 27.86 |
| ATOM | 2696 | CD | PRO | A | 372 | 76.803 | 31.069 | 34.108 | 1.00 | 28.58 |
| ATOM | 2697 | CA | PRO | A | 372 | 74.879 | 29.570 | 34.295 | 1.00 | 28.30 |
| ATOM | 2698 | CB | PRO | A | 372 | 74.857 | 30.622 | 35.415 | 1.00 | 28.19 |
| ATOM | 2699 | CG | PRO | A | 372 | 76.266 | 31.086 | 35.518 | 1.00 | 29.14 |
| ATOM | 2700 | C | PRO | A | 372 | 73.564 | 29.642 | 33.508 | 1.00 | 28.82 |
| ATOM | 2701 | O | PRO | A | 372 | 72.582 | 28.953 | 33.834 | 1.00 | 27.79 |
| ATOM | 2702 | N | LEU | A | 373 | 73.544 | 30.479 | 32.466 | 1.00 | 28.69 |
| ATOM | 2703 | CA | LEU | A | 373 | 72.353 | 30.643 | 31.627 | 1.00 | 29.17 |
| ATOM | 2704 | CB | LEU | A | 373 | 72.618 | 31.651 | 30.508 | 1.00 | 29.53 |
| ATOM | 2705 | CG | LEU | A | 373 | 72.437 | 33.126 | 30.866 | 1.00 | 30.15 |
| ATOM | 2706 | CD1 | LEU | A | 373 | 72.479 | 33.987 | 29.604 | 1.00 | 31.19 |
| ATOM | 2707 | CD2 | LEU | A | 373 | 71.139 | 33.342 | 31.623 | 1.00 | 26.78 |
| ATOM | 2708 | C | LEU | A | 373 | 71.871 | 29.314 | 31.035 | 1.00 | 27.62 |
| ATOM | 2709 | O | LEU | A | 373 | 70.722 | 29.202 | 30.623 | 1.00 | 28.74 |
| ATOM | 2710 | N | ALA | A | 374 | 72.754 | 28.314 | 31.005 | 1.00 | 28.72 |
| ATOM | 2711 | CA | ALA | A | 374 | 72.438 | 26.981 | 30.471 | 1.00 | 30.45 |
| ATOM | 2712 | CB | ALA | A | 374 | 73.660 | 26.075 | 30.561 | 1.00 | 30.45 |
| ATOM | 2713 | C | ALA | A | 374 | 71.227 | 26.313 | 31.142 | 1.00 | 31.42 |
| ATOM | 2714 | O | ALA | A | 374 | 70.521 | 25.522 | 30.507 | 1.00 | 31.67 |
| ATOM | 2715 | N | THR | A | 375 | 70.988 | 26.613 | 32.424 | 1.00 | 32.66 |
| ATOM | 2716 | CA | THR | A | 375 | 69.860 | 26.010 | 33.149 | 1.00 | 32.06 |
| ATOM | 2717 | CB | THR | A | 375 | 69.942 | 26.279 | 34.659 | 1.00 | 33.87 |
| ATOM | 2718 | OG1 | THR | A | 375 | 69.884 | 27.667 | 34.951 | 1.00 | 36.94 |
| ATOM | 2719 | CG2 | THR | A | 375 | 71.191 | 25.720 | 35.301 | 1.00 | 33.69 |
| ATOM | 2720 | C | THR | A | 375 | 68.518 | 26.438 | 32.567 | 1.00 | 31.74 |
| ATOM | 2721 | O | THR | A | 375 | 67.512 | 25.734 | 32.692 | 1.00 | 31.00 |
| ATOM | 2722 | N | ILE | A | 376 | 68.513 | 27.581 | 31.898 | 1.00 | 32.30 |
| ATOM | 2723 | CA | ILE | A | 376 | 67.300 | 28.075 | 31.261 | 1.00 | 32.02 |
| ATOM | 2724 | CB | ILE | A | 376 | 67.176 | 29.614 | 31.381 | 1.00 | 33.14 |
| ATOM | 2725 | CG2 | ILE | A | 376 | 66.145 | 30.159 | 30.401 | 1.00 | 33.69 |
| ATOM | 2726 | CG1 | ILE | A | 376 | 66.805 | 30.016 | 32.806 | 1.00 | 31.53 |
| ATOM | 2727 | CD1 | ILE | A | 376 | 67.100 | 31.466 | 33.115 | 1.00 | 31.64 |
| ATOM | 2728 | C | ILE | A | 376 | 67.285 | 27.682 | 29.776 | 1.00 | 30.98 |
| ATOM | 2729 | O | ILE | A | 376 | 66.274 | 27.222 | 29.257 | 1.00 | 31.62 |
| ATOM | 2730 | N | LEU | A | 377 | 68.416 | 27.903 | 29.108 | 1.00 | 31.31 |
| ATOM | 2731 | CA | LEU | A | 377 | 68.582 | 27.635 | 27.672 | 1.00 | 31.31 |
| ATOM | 2732 | CB | LEU | A | 377 | 69.931 | 28.161 | 27.191 | 1.00 | 27.99 |
| ATOM | 2733 | CG | LEU | A | 377 | 70.072 | 29.675 | 27.259 | 1.00 | 25.47 |
| ATOM | 2734 | CD1 | LEU | A | 377 | 71.528 | 30.100 | 27.237 | 1.00 | 23.87 |
| ATOM | 2735 | CD2 | LEU | A | 377 | 69.280 | 30.325 | 26.145 | 1.00 | 24.82 |
| ATOM | 2736 | C | LEU | A | 377 | 68.397 | 26.176 | 27.274 | 1.00 | 32.58 |
| ATOM | 2737 | O | LEU | A | 377 | 67.801 | 25.887 | 26.231 | 1.00 | 32.49 |
| ATOM | 2738 | N | ILE | A | 378 | 68.920 | 25.264 | 28.092 | 1.00 | 33.25 |
| ATOM | 2739 | CA | ILE | A | 378 | 68.822 | 23.840 | 27.811 | 1.00 | 32.83 |
| ATOM | 2740 | CB | ILE | A | 378 | 70.186 | 23.137 | 27.968 | 1.00 | 30.55 |
| ATOM | 2741 | CG2 | ILE | A | 378 | 70.072 | 21.663 | 27.607 | 1.00 | 30.53 |
| ATOM | 2742 | CG1 | ILE | A | 378 | 71.238 | 23.830 | 27.090 | 1.00 | 29.45 |
| ATOM | 2743 | CD1 | ILE | A | 378 | 72.659 | 23.356 | 27.321 | 1.00 | 28.60 |
| ATOM | 2744 | C | ILE | A | 378 | 67.771 | 23.171 | 28.697 | 1.00 | 35.98 |
| ATOM | 2745 | O | ILE | A | 378 | 68.046 | 22.821 | 29.849 | 1.00 | 36.93 |
| ATOM | 2746 | N | PRO | A | 379 | 66.544 | 22.982 | 28.162 | 1.00 | 36.15 |
| ATOM | 2747 | CD | PRO | A | 379 | 66.146 | 23.369 | 26.796 | 1.00 | 36.55 |
| ATOM | 2748 | CA | PRO | A | 379 | 65.447 | 22.345 | 28.899 | 1.00 | 36.01 |
| ATOM | 2749 | CB | PRO | A | 379 | 64.261 | 22.395 | 27.925 | 1.00 | 35.91 |
| ATOM | 2750 | CG | PRO | A | 379 | 64.863 | 22.617 | 26.579 | 1.00 | 36.09 |
| ATOM | 2751 | C | PRO | A | 379 | 65.782 | 20.902 | 29.272 | 1.00 | 36.21 |
| ATOM | 2752 | O | PRO | A | 379 | 66.574 | 20.251 | 28.587 | 1.00 | 37.18 |
| ATOM | 2753 | N | PRO | A | 380 | 65.192 | 20.394 | 30.387 | 1.00 | 36.38 |
| ATOM | 2754 | CD | PRO | A | 380 | 64.257 | 21.124 | 31.257 | 1.00 | 35.79 |
| ATOM | 2755 | CA | PRO | A | 380 | 65.431 | 19.029 | 30.895 | 1.00 | 35.56 |
| ATOM | 2756 | CB | PRO | A | 380 | 64.366 | 18.844 | 31.990 | 1.00 | 35.32 |
| ATOM | 2757 | CG | PRO | A | 380 | 63.444 | 20.013 | 31.859 | 1.00 | 35.77 |
| ATOM | 2758 | C | PRO | A | 380 | 65.319 | 17.928 | 29.836 | 1.00 | 35.35 |
| ATOM | 2759 | O | PRO | A | 380 | 66.174 | 17.045 | 29.770 | 1.00 | 35.04 |
| ATOM | 2760 | N | HIS | A | 381 | 64.269 | 17.975 | 29.019 | 1.00 | 36.71 |
| ATOM | 2761 | CA | HIS | A | 381 | 64.055 | 16.967 | 27.977 | 1.00 | 38.77 |
| ATOM | 2762 | CB | HIS | A | 381 | 62.675 | 17.142 | 27.341 | 1.00 | 39.92 |
| ATOM | 2763 | CG | HIS | A | 381 | 62.575 | 18.337 | 26.441 | 1.00 | 40.52 |
| ATOM | 2764 | CD2 | HIS | A | 381 | 62.183 | 19.613 | 26.696 | 1.00 | 40.13 |
| ATOM | 2765 | ND1 | HIS | A | 381 | 62.924 | 18.305 | 25.105 | 1.00 | 40.43 |
| ATOM | 2766 | CE1 | HIS | A | 381 | 62.754 | 19.508 | 24.578 | 1.00 | 39.78 |
| ATOM | 2767 | NE2 | HIS | A | 381 | 62.305 | 20.319 | 25.523 | 1.00 | 39.78 |
| ATOM | 2768 | C | HIS | A | 381 | 65.135 | 16.982 | 26.886 | 1.00 | 40.47 |
| ATOM | 2769 | O | HIS | A | 381 | 65.301 | 15.998 | 26.165 | 1.00 | 41.54 |
| ATOM | 2770 | N | ALA | A | 382 | 65.845 | 18.102 | 26.752 | 1.00 | 41.62 |
| ATOM | 2771 | CA | ALA | A | 382 | 66.883 | 18.250 | 25.729 | 1.00 | 43.15 |
| ATOM | 2772 | CB | ALA | A | 382 | 66.857 | 19.662 | 25.151 | 1.00 | 42.23 |
| ATOM | 2773 | C | ALA | A | 382 | 68.281 | 17.921 | 26.250 | 1.00 | 44.64 |
| ATOM | 2774 | O | ALA | A | 382 | 69.198 | 17.672 | 25.460 | 1.00 | 44.18 |
| ATOM | 2775 | N | ARG | A | 383 | 68.454 | 17.934 | 27.572 | 1.00 | 46.22 |
| ATOM | 2776 | CA | ARG | A | 383 | 69.757 | 17.648 | 28.167 | 1.00 | 47.97 |
| ATOM | 2777 | CB | ARG | A | 383 | 69.736 | 17.948 | 29.663 | 1.00 | 48.04 |
| ATOM | 2778 | CG | ARG | A | 383 | 69.744 | 19.445 | 29.958 | 1.00 | 49.84 |
| ATOM | 2779 | CD | ARG | A | 383 | 69.740 | 19.742 | 31.451 | 1.00 | 49.31 |
| ATOM | 2780 | NE | ARG | A | 383 | 68.841 | 20.849 | 31.789 | 1.00 | 50.09 |
| ATOM | 2781 | CZ | ARG | A | 383 | 68.325 | 21.059 | 33.011 | 1.00 | 51.14 |
| ATOM | 2782 | NH1 | ARG | A | 383 | 68.671 | 20.282 | 34.040 | 1.00 | 50.04 |
| ATOM | 2783 | NH2 | ARG | A | 383 | 67.467 | 22.059 | 33.207 | 1.00 | 50.78 |
| ATOM | 2784 | C | ARG | A | 383 | 70.202 | 16.208 | 27.888 | 1.00 | 49.14 |
| ATOM | 2785 | O | ARG | A | 383 | 69.405 | 15.269 | 27.951 | 1.00 | 49.60 |
| ATOM | 2786 | N | ILE | A | 384 | 71.485 | 16.064 | 27.544 | 1.00 | 50.06 |
| ATOM | 2787 | CA | ILE | A | 384 | 72.086 | 14.770 | 27.210 | 1.00 | 51.09 |
| ATOM | 2788 | CB | ILE | A | 384 | 72.710 | 14.801 | 25.795 | 1.00 | 51.18 |
| ATOM | 2789 | CG2 | ILE | A | 384 | 72.959 | 13.393 | 25.280 | 1.00 | 52.93 |
| ATOM | 2790 | CG1 | ILE | A | 384 | 71.819 | 15.577 | 24.825 | 1.00 | 50.79 |
| ATOM | 2791 | CD1 | ILE | A | 384 | 72.542 | 16.695 | 24.105 | 1.00 | 52.35 |
| ATOM | 2792 | C | ILE | A | 384 | 73.170 | 14.372 | 28.219 | 1.00 | 51.65 |
| ATOM | 2793 | OT1 | ILE | A | 384 | 73.644 | 15.255 | 28.970 | 1.00 | 51.89 |
| ATOM | 2794 | OXT | ILE | A | 384 | 73.537 | 13.175 | 28.250 | 1.00 | 52.45 |
| TER | 2795 | | ILE | A | 384 | | | | | |
| ATOM | 2796 | CB | VAL | B | 37 | 36.802 | 77.140 | −23.975 | 1.00 | 51.55 |
| ATOM | 2797 | CG1 | VAL | B | 37 | 35.939 | 76.007 | −23.436 | 1.00 | 50.87 |
| ATOM | 2798 | CG2 | VAL | B | 37 | 35.933 | 78.188 | −24.660 | 1.00 | 51.89 |
| ATOM | 2799 | C | VAL | B | 37 | 38.541 | 76.737 | −22.201 | 1.00 | 51.79 |
| ATOM | 2800 | O | VAL | B | 37 | 39.596 | 76.390 | −22.744 | 1.00 | 52.10 |
| ATOM | 2801 | N | VAL | B | 37 | 38.437 | 78.942 | −23.333 | 1.00 | 51.63 |
| ATOM | 2802 | CA | VAL | B | 37 | 37.637 | 77.786 | −22.844 | 1.00 | 51.54 |
| ATOM | 2803 | N | THR | B | 38 | 38.120 | 76.245 | −21.032 | 1.00 | 51.45 |
| ATOM | 2804 | CA | THR | B | 38 | 38.872 | 75.239 | −20.274 | 1.00 | 49.87 |
| ATOM | 2805 | CB | THR | B | 38 | 38.764 | 73.832 | −20.899 | 1.00 | 50.03 |
| ATOM | 2806 | OG1 | THR | B | 38 | 39.783 | 72.969 | −20.422 | 1.00 | 50.26 |
| ATOM | 2807 | CG2 | THR | B | 38 | 38.796 | 73.804 | −22.412 | 1.00 | 50.44 |
| ATOM | 2808 | C | THR | B | 38 | 40.329 | 75.658 | −20.045 | 1.00 | 48.71 |
| ATOM | 2809 | O | THR | B | 38 | 41.244 | 75.175 | −20.712 | 1.00 | 49.59 |
| ATOM | 2810 | N | THR | B | 39 | 40.526 | 76.558 | −19.083 | 1.00 | 46.30 |
| ATOM | 2811 | CA | THR | B | 39 | 41.852 | 77.053 | −18.729 | 1.00 | 43.19 |
| ATOM | 2812 | CB | THR | B | 39 | 41.750 | 78.464 | −18.147 | 1.00 | 43.29 |
| ATOM | 2813 | OG1 | THR | B | 39 | 40.832 | 79.264 | −18.874 | 1.00 | 44.92 |
| ATOM | 2814 | CG2 | THR | B | 39 | 43.072 | 79.196 | −18.077 | 1.00 | 43.86 |
| ATOM | 2815 | C | THR | B | 39 | 42.495 | 76.141 | −17.695 | 1.00 | 41.22 |
| ATOM | 2816 | O | THR | B | 39 | 41.838 | 75.709 | −16.753 | 1.00 | 42.40 |
| ATOM | 2817 | N | VAL | B | 40 | 43.783 | 75.871 | −17.864 | 1.00 | 38.57 |
| ATOM | 2818 | CA | VAL | B | 40 | 44.520 | 75.032 | −16.930 | 1.00 | 35.10 |
| ATOM | 2819 | CB | VAL | B | 40 | 45.089 | 73.765 | −17.593 | 1.00 | 34.50 |
| ATOM | 2820 | CG1 | VAL | B | 40 | 45.866 | 72.929 | −16.583 | 1.00 | 34.63 |
| ATOM | 2821 | CG2 | VAL | B | 40 | 43.965 | 72.949 | −18.199 | 1.00 | 32.73 |
| ATOM | 2822 | C | VAL | B | 40 | 45.632 | 75.836 | −16.278 | 1.00 | 34.58 |
| ATOM | 2823 | O | VAL | B | 40 | 46.470 | 76.440 | −16.961 | 1.00 | 34.85 |
| ATOM | 2824 | N | VAL | B | 41 | 45.616 | 75.871 | −14.953 | 1.00 | 31.60 |
| ATOM | 2825 | CA | VAL | B | 41 | 46.603 | 76.629 | −14.205 | 1.00 | 29.68 |
| ATOM | 2826 | CB | VAL | B | 41 | 45.963 | 77.919 | −13.625 | 1.00 | 30.25 |
| ATOM | 2827 | CG1 | VAL | B | 41 | 45.113 | 77.614 | −12.395 | 1.00 | 30.02 |
| ATOM | 2828 | CG2 | VAL | B | 41 | 47.023 | 78.955 | −13.300 | 1.00 | 31.38 |
| ATOM | 2829 | C | VAL | B | 41 | 47.175 | 75.798 | −13.066 | 1.00 | 27.96 |
| ATOM | 2830 | O | VAL | B | 41 | 46.525 | 74.886 | −12.576 | 1.00 | 26.60 |
| ATOM | 2831 | N | ALA | B | 42 | 48.373 | 76.145 | −12.630 | 1.00 | 28.15 |
| ATOM | 2832 | CA | ALA | B | 42 | 48.993 | 75.450 | −11.505 | 1.00 | 29.54 |
| ATOM | 2833 | CB | ALA | B | 42 | 50.484 | 75.244 | −11.733 | 1.00 | 28.92 |
| ATOM | 2834 | C | ALA | B | 42 | 48.742 | 76.280 | −10.249 | 1.00 | 29.48 |
| ATOM | 2835 | O | ALA | B | 42 | 49.269 | 77.381 | −10.122 | 1.00 | 28.42 |
| ATOM | 2836 | N | THR | B | 43 | 47.904 | 75.767 | −9.349 | 1.00 | 30.71 |
| ATOM | 2837 | CA | THR | B | 43 | 47.558 | 76.496 | −8.120 | 1.00 | 33.11 |
| ATOM | 2838 | CB | THR | B | 43 | 46.168 | 77.126 | −8.288 | 1.00 | 35.28 |
| ATOM | 2839 | OG1 | THR | B | 43 | 45.614 | 77.507 | −7.040 | 1.00 | 42.46 |
| ATOM | 2840 | CG2 | THR | B | 43 | 45.166 | 76.232 | −8.980 | 1.00 | 35.35 |
| ATOM | 2841 | C | THR | B | 43 | 47.569 | 75.605 | −6.871 | 1.00 | 32.03 |
| ATOM | 2842 | O | THR | B | 43 | 47.210 | 74.437 | −6.942 | 1.00 | 31.25 |
| ATOM | 2843 | N | PRO | B | 44 | 48.002 | 76.153 | −5.704 | 1.00 | 32.19 |
| ATOM | 2844 | CD | PRO | B | 44 | 48.501 | 77.503 | −5.521 | 1.00 | 34.09 |
| ATOM | 2845 | CA | PRO | B | 44 | 48.067 | 75.431 | −4.431 | 1.00 | 32.79 |
| ATOM | 2846 | CB | PRO | B | 44 | 49.192 | 76.165 | −3.672 | 1.00 | 32.20 |
| ATOM | 2847 | CG | PRO | B | 44 | 49.639 | 77.267 | −4.578 | 1.00 | 31.97 |
| ATOM | 2848 | C | PRO | B | 44 | 46.773 | 75.558 | −3.632 | 1.00 | 31.84 |
| ATOM | 2849 | O | PRO | B | 44 | 46.099 | 76.586 | −3.680 | 1.00 | 28.42 |
| ATOM | 2850 | N | GLY | B | 45 | 46.452 | 74.512 | −2.872 | 1.00 | 35.47 |
| ATOM | 2851 | CA | GLY | B | 45 | 45.257 | 74.538 | −2.036 | 1.00 | 39.23 |
| ATOM | 2852 | C | GLY | B | 45 | 45.504 | 75.287 | −0.729 | 1.00 | 41.21 |
| ATOM | 2853 | O | GLY | B | 45 | 46.462 | 76.060 | −0.623 | 1.00 | 38.55 |
| ATOM | 2854 | N | GLN | B | 46 | 44.650 | 75.046 | 0.274 | 1.00 | 46.75 |
| ATOM | 2855 | CA | GLN | B | 46 | 44.793 | 75.693 | 1.588 | 1.00 | 48.74 |
| ATOM | 2856 | CB | GLN | B | 46 | 43.484 | 76.341 | 2.035 | 1.00 | 50.72 |
| ATOM | 2857 | CG | GLN | B | 46 | 42.261 | 75.448 | 1.895 | 1.00 | 53.41 |
| ATOM | 2858 | CD | GLN | B | 46 | 41.122 | 75.883 | 2.795 | 1.00 | 53.10 |
| ATOM | 2859 | OE1 | GLN | B | 46 | 40.767 | 77.064 | 2.841 | 1.00 | 53.10 |
| ATOM | 2860 | NE2 | GLN | B | 46 | 40.544 | 74.926 | 3.518 | 1.00 | 53.76 |
| ATOM | 2861 | C | GLN | B | 46 | 45.320 | 74.716 | 2.647 | 1.00 | 50.13 |
| ATOM | 2862 | O | GLN | B | 46 | 44.619 | 74.359 | 3.603 | 1.00 | 50.65 |
| ATOM | 2863 | N | GLY | B | 47 | 46.570 | 74.298 | 2.458 | 1.00 | 50.48 |
| ATOM | 2864 | CA | GLY | B | 47 | 47.236 | 73.375 | 3.366 | 1.00 | 51.37 |
| ATOM | 2865 | C | GLY | B | 47 | 48.740 | 73.407 | 3.156 | 1.00 | 51.41 |
| ATOM | 2866 | O | GLY | B | 47 | 49.328 | 74.487 | 3.092 | 1.00 | 52.08 |
| ATOM | 2867 | N | PRO | B | 48 | 49.339 | 72.184 | 2.925 | 1.00 | 51.20 |
| ATOM | 2868 | CD | PRO | B | 48 | 49.147 | 71.119 | 3.933 | 1.00 | 51.98 |
| ATOM | 2869 | CA | PRO | B | 48 | 50.809 | 71.950 | 2.537 | 1.00 | 50.93 |
| ATOM | 2870 | CB | PRO | B | 48 | 50.961 | 70.469 | 2.445 | 1.00 | 51.77 |
| ATOM | 2871 | CG | PRO | B | 48 | 50.174 | 70.062 | 3.621 | 1.00 | 51.47 |
| ATOM | 2872 | C | PRO | B | 48 | 51.163 | 72.527 | 1.240 | 1.00 | 49.84 |
| ATOM | 2873 | O | PRO | B | 48 | 51.079 | 71.800 | 0.266 | 1.00 | 50.79 |
| ATOM | 2874 | N | ASP | B | 49 | 51.537 | 73.731 | 1.055 | 1.00 | 48.06 |
| ATOM | 2875 | CA | ASP | B | 49 | 51.732 | 74.195 | −0.333 | 1.00 | 47.60 |
| ATOM | 2876 | CB | ASP | B | 49 | 53.022 | 74.936 | −0.423 | 1.00 | 48.55 |
| ATOM | 2877 | CG | ASP | B | 49 | 52.835 | 76.305 | 0.221 | 1.00 | 50.19 |
| ATOM | 2878 | OD1 | ASP | B | 49 | 52.171 | 77.158 | −0.396 | 1.00 | 52.20 |
| ATOM | 2879 | OD2 | ASP | B | 49 | 53.335 | 76.504 | 1.351 | 1.00 | 51.50 |
| ATOM | 2880 | C | ASP | B | 49 | 51.479 | 73.199 | −1.498 | 1.00 | 45.81 |
| ATOM | 2881 | O | ASP | B | 49 | 50.386 | 73.287 | −2.046 | 1.00 | 45.95 |
| ATOM | 2882 | N | ARG | B | 50 | 52.319 | 72.257 | −1.958 | 1.00 | 42.31 |
| ATOM | 2883 | CA | ARG | B | 50 | 51.892 | 71.327 | −3.084 | 1.00 | 40.48 |
| ATOM | 2884 | CB | ARG | B | 50 | 51.271 | 70.083 | −2.442 | 1.00 | 41.47 |
| ATOM | 2885 | CG | ARG | B | 50 | 50.732 | 69.037 | −3.397 | 1.00 | 44.10 |
| ATOM | 2886 | CD | ARG | B | 50 | 50.762 | 67.634 | −2.780 | 1.00 | 46.35 |
| ATOM | 2887 | NE | ARG | B | 50 | 49.679 | 67.438 | −1.813 | 1.00 | 51.22 |
| ATOM | 2888 | CZ | ARG | B | 50 | 49.855 | 67.035 | −0.542 | 1.00 | 51.26 |
| ATOM | 2889 | NH1 | ARG | B | 50 | 51.069 | 66.787 | −0.075 | 1.00 | 51.16 |
| ATOM | 2890 | NH2 | ARG | B | 50 | 48.804 | 66.876 | 0.261 | 1.00 | 51.87 |
| ATOM | 2891 | C | ARG | B | 50 | 50.922 | 71.887 | −4.203 | 1.00 | 35.73 |
| ATOM | 2892 | O | ARG | B | 50 | 49.752 | 71.523 | −4.252 | 1.00 | 34.77 |
| ATOM | 2893 | N | PRO | B | 51 | 51.446 | 72.766 | −5.084 | 1.00 | 33.78 |
| ATOM | 2894 | CD | PRO | B | 51 | 52.801 | 73.315 | −4.963 | 1.00 | 33.89 |
| ATOM | 2895 | CA | PRO | B | 51 | 50.736 | 73.310 | −6.258 | 1.00 | 33.19 |
| ATOM | 2896 | CB | PRO | B | 51 | 51.750 | 74.263 | −6.894 | 1.00 | 31.56 |
| ATOM | 2897 | CG | PRO | B | 51 | 52.736 | 74.552 | −5.816 | 1.00 | 33.65 |
| ATOM | 2898 | C | PRO | B | 51 | 50.373 | 72.199 | −7.238 | 1.00 | 31.20 |
| ATOM | 2899 | O | PRO | B | 51 | 51.215 | 71.378 | −7.590 | 1.00 | 33.41 |
| ATOM | 2900 | N | GLN | B | 52 | 49.115 | 72.159 | −7.645 | 1.00 | 30.94 |
| ATOM | 2901 | CA | GLN | B | 52 | 48.631 | 71.128 | −8.559 | 1.00 | 30.72 |
| ATOM | 2902 | CB | GLN | B | 52 | 47.668 | 70.192 | −7.822 | 1.00 | 32.26 |
| ATOM | 2903 | CG | GLN | B | 52 | 46.545 | 70.921 | −7.099 | 1.00 | 34.16 |
| ATOM | 2904 | CD | GLN | B | 52 | 45.411 | 70.001 | −6.697 | 1.00 | 37.58 |
| ATOM | 2905 | OE1 | GLN | B | 52 | 44.489 | 69.751 | −7.480 | 1.00 | 39.21 |
| ATOM | 2906 | NE2 | GLN | B | 52 | 45.466 | 69.492 | −5.466 | 1.00 | 38.21 |
| ATOM | 2907 | C | GLN | B | 52 | 47.933 | 71.726 | −9.785 | 1.00 | 29.17 |
| ATOM | 2908 | O | GLN | B | 52 | 47.461 | 72.863 | −9.763 | 1.00 | 26.63 |
| ATOM | 2909 | N | GLU | B | 53 | 47.858 | 70.940 | −10.851 | 1.00 | 29.44 |
| ATOM | 2910 | CA | GLU | B | 53 | 47.206 | 71.378 | −12.086 | 1.00 | 29.61 |
| ATOM | 2911 | CB | GLU | B | 53 | 47.528 | 70.400 | −13.220 | 1.00 | 30.96 |
| ATOM | 2912 | CG | GLU | B | 53 | 48.757 | 70.782 | −14.031 | 1.00 | 33.49 |
| ATOM | 2913 | CD | GLU | B | 53 | 48.861 | 70.022 | −15.351 | 1.00 | 36.32 |
| ATOM | 2914 | OE1 | GLU | B | 53 | 49.955 | 69.489 | −15.639 | 1.00 | 35.83 |
| ATOM | 2915 | OE2 | GLU | B | 53 | 47.849 | 69.963 | −16.098 | 1.00 | 36.83 |
| ATOM | 2916 | C | GLU | B | 53 | 45.693 | 71.502 | −11.900 | 1.00 | 28.07 |
| ATOM | 2917 | O | GLU | B | 53 | 45.045 | 70.562 | −11.459 | 1.00 | 29.41 |
| ATOM | 2918 | N | VAL | B | 54 | 45.138 | 72.674 | −12.234 | 1.00 | 27.34 |
| ATOM | 2919 | CA | VAL | B | 54 | 43.701 | 72.922 | −12.096 | 1.00 | 27.42 |
| ATOM | 2920 | CB | VAL | B | 54 | 43.395 | 73.879 | −10.921 | 1.00 | 26.77 |
| ATOM | 2921 | CG1 | VAL | B | 54 | 41.970 | 74.411 | −11.018 | 1.00 | 26.06 |
| ATOM | 2922 | CG2 | VAL | B | 54 | 43.608 | 73.179 | −9.586 | 1.00 | 28.23 |
| ATOM | 2923 | C | VAL | B | 54 | 43.110 | 73.507 | −13.382 | 1.00 | 28.07 |
| ATOM | 2924 | O | VAL | B | 54 | 43.619 | 74.490 | −13.924 | 1.00 | 30.35 |
| ATOM | 2925 | N | SER | B | 55 | 42.033 | 72.900 | −13.861 | 1.00 | 28.47 |
| ATOM | 2926 | CA | SER | B | 55 | 41.378 | 73.363 | −15.083 | 1.00 | 29.26 |
| ATOM | 2927 | CB | SER | B | 55 | 41.358 | 72.252 | −16.140 | 1.00 | 27.83 |
| ATOM | 2928 | OG | SER | B | 55 | 42.566 | 71.509 | −16.135 | 1.00 | 30.97 |
| ATOM | 2929 | C | SER | B | 55 | 39.963 | 73.853 | −14.815 | 1.00 | 28.63 |
| ATOM | 2930 | O | SER | B | 55 | 39.209 | 73.238 | −14.063 | 1.00 | 29.48 |
| ATOM | 2931 | N | TYR | B | 56 | 39.600 | 74.958 | −15.447 | 1.00 | 29.65 |
| ATOM | 2932 | CA | TYR | B | 56 | 38.267 | 75.513 | −15.286 | 1.00 | 31.29 |
| ATOM | 2933 | CB | TYR | B | 56 | 38.209 | 76.490 | −14.110 | 1.00 | 29.12 |
| ATOM | 2934 | CG | TYR | B | 56 | 39.108 | 77.699 | −14.233 | 1.00 | 26.96 |
| ATOM | 2935 | CD1 | TYR | B | 56 | 40.371 | 77.712 | −13.651 | 1.00 | 27.72 |
| ATOM | 2936 | CE1 | TYR | B | 56 | 41.183 | 78.825 | −13.737 | 1.00 | 27.52 |
| ATOM | 2937 | CD2 | TYR | B | 56 | 38.686 | 78.833 | −14.903 | 1.00 | 24.94 |
| ATOM | 2938 | CE2 | TYR | B | 56 | 39.495 | 79.947 | −14.998 | 1.00 | 26.38 |
| ATOM | 2939 | CZ | TYR | B | 56 | 40.740 | 79.940 | −14.413 | 1.00 | 26.96 |
| ATOM | 2940 | OH | TYR | B | 56 | 41.544 | 81.057 | −14.497 | 1.00 | 29.38 |
| ATOM | 2941 | C | TYR | B | 56 | 37.792 | 76.184 | −16.561 | 1.00 | 32.84 |
| ATOM | 2942 | O | TYR | B | 56 | 38.585 | 76.736 | −17.311 | 1.00 | 34.73 |
| ATOM | 2943 | N | THR | B | 57 | 36.495 | 76.137 | −16.798 | 1.00 | 35.16 |
| ATOM | 2944 | CA | THR | B | 57 | 35.925 | 76.747 | −17.988 | 1.00 | 37.24 |
| ATOM | 2945 | CB | THR | B | 57 | 35.620 | 75.668 | −19.030 | 1.00 | 36.42 |
| ATOM | 2946 | OG1 | THR | B | 57 | 35.393 | 76.247 | −20.300 | 1.00 | 38.30 |
| ATOM | 2947 | CG2 | THR | B | 57 | 34.434 | 74.796 | −18.685 | 1.00 | 34.17 |
| ATOM | 2948 | C | THR | B | 57 | 34.665 | 77.545 | −17.667 | 1.00 | 38.22 |
| ATOM | 2949 | O | THR | B | 57 | 34.196 | 77.556 | −16.529 | 1.00 | 38.82 |
| ATOM | 2950 | N | ASP | B | 58 | 34.117 | 78.198 | −18.691 | 1.00 | 38.92 |
| ATOM | 2951 | CA | ASP | B | 58 | 32.898 | 78.991 | −18.557 | 1.00 | 38.50 |
| ATOM | 2952 | CB | ASP | B | 58 | 31.737 | 78.118 | −18.060 | 1.00 | 40.09 |
| ATOM | 2953 | CG | ASP | B | 58 | 31.472 | 76.911 | −18.950 | 1.00 | 41.34 |
| ATOM | 2954 | OD1 | ASP | B | 58 | 30.854 | 75.941 | −18.464 | 1.00 | 42.69 |
| ATOM | 2955 | OD2 | ASP | B | 58 | 31.878 | 76.934 | −20.133 | 1.00 | 43.89 |
| ATOM | 2956 | C | ASP | B | 58 | 33.100 | 80.211 | −17.660 | 1.00 | 38.07 |
| ATOM | 2957 | O | ASP | B | 58 | 32.332 | 80.445 | −16.729 | 1.00 | 39.03 |
| ATOM | 2958 | N | THR | B | 59 | 34.137 | 80.984 | −17.960 | 1.00 | 38.28 |
| ATOM | 2959 | CA | THR | B | 59 | 34.465 | 82.187 | −17.209 | 1.00 | 39.15 |
| ATOM | 2960 | CB | THR | B | 59 | 35.838 | 82.701 | −17.622 | 1.00 | 38.09 |
| ATOM | 2961 | OG1 | THR | B | 59 | 36.825 | 81.710 | −17.404 | 1.00 | 38.32 |
| ATOM | 2962 | CG2 | THR | B | 59 | 36.263 | 83.960 | −16.897 | 1.00 | 40.41 |
| ATOM | 2963 | C | THR | B | 59 | 33.401 | 83.263 | −17.411 | 1.00 | 40.95 |
| ATOM | 2964 | O | THR | B | 59 | 32.946 | 83.499 | −18.535 | 1.00 | 43.05 |
| ATOM | 2965 | N | LYS | B | 60 | 32.988 | 83.897 | −16.315 | 1.00 | 41.08 |
| ATOM | 2966 | CA | LYS | B | 60 | 31.949 | 84.922 | −16.372 | 1.00 | 39.78 |
| ATOM | 2967 | CB | LYS | B | 60 | 30.588 | 84.226 | −16.264 | 1.00 | 39.82 |
| ATOM | 2968 | CG | LYS | B | 60 | 29.422 | 85.144 | −15.953 | 1.00 | 40.01 |
| ATOM | 2969 | CD | LYS | B | 60 | 28.092 | 84.454 | −16.237 | 1.00 | 40.00 |
| ATOM | 2970 | CE | LYS | B | 60 | 27.314 | 84.172 | −14.961 | 1.00 | 40.98 |
| ATOM | 2971 | NZ | LYS | B | 60 | 26.271 | 85.203 | −14.710 | 1.00 | 42.05 |
| ATOM | 2972 | C | LYS | B | 60 | 32.121 | 85.947 | −15.255 | 1.00 | 39.73 |
| ATOM | 2973 | O | LYS | B | 60 | 32.221 | 85.585 | −14.083 | 1.00 | 40.11 |
| ATOM | 2974 | N | VAL | B | 61 | 32.146 | 87.229 | −15.621 | 1.00 | 39.35 |
| ATOM | 2975 | CA | VAL | B | 61 | 32.297 | 88.292 | −14.631 | 1.00 | 37.83 |
| ATOM | 2976 | CB | VAL | B | 61 | 32.684 | 89.640 | −15.272 | 1.00 | 37.73 |
| ATOM | 2977 | CG1 | VAL | B | 61 | 32.348 | 90.802 | −14.347 | 1.00 | 37.25 |
| ATOM | 2978 | CG2 | VAL | B | 61 | 34.167 | 89.649 | −15.620 | 1.00 | 36.79 |
| ATOM | 2979 | C | VAL | B | 61 | 31.025 | 88.442 | −13.805 | 1.00 | 37.38 |
| ATOM | 2980 | O | VAL | B | 61 | 29.979 | 88.845 | −14.320 | 1.00 | 36.86 |
| ATOM | 2981 | N | ILE | B | 62 | 31.126 | 88.100 | −12.524 | 1.00 | 35.32 |
| ATOM | 2982 | CA | ILE | B | 62 | 29.993 | 88.177 | −11.613 | 1.00 | 33.49 |
| ATOM | 2983 | CB | ILE | B | 62 | 29.849 | 86.898 | −10.774 | 1.00 | 34.06 |
| ATOM | 2984 | CG2 | ILE | B | 62 | 29.257 | 85.777 | −11.616 | 1.00 | 33.69 |
| ATOM | 2985 | CG1 | ILE | B | 62 | 31.200 | 86.490 | −10.183 | 1.00 | 32.38 |
| ATOM | 2986 | CD1 | ILE | B | 62 | 31.092 | 85.548 | −9.009 | 1.00 | 31.62 |
| ATOM | 2987 | C | ILE | B | 62 | 30.070 | 89.381 | −10.676 | 1.00 | 31.70 |
| ATOM | 2988 | O | ILE | B | 62 | 29.042 | 89.855 | −10.203 | 1.00 | 32.94 |
| ATOM | 2989 | N | GLY | B | 63 | 31.279 | 89.865 | −10.400 | 1.00 | 30.66 |
| ATOM | 2990 | CA | GLY | B | 63 | 31.414 | 91.001 | −9.505 | 1.00 | 29.23 |
| ATOM | 2991 | C | GLY | B | 63 | 32.670 | 91.809 | −9.732 | 1.00 | 29.90 |
| ATOM | 2992 | O | GLY | B | 63 | 33.393 | 91.593 | −10.708 | 1.00 | 31.54 |
| ATOM | 2993 | N | ASN | B | 64 | 32.927 | 92.756 | −8.827 | 1.00 | 30.27 |
| ATOM | 2994 | CA | ASN | B | 64 | 34.103 | 93.619 | −8.924 | 1.00 | 31.14 |
| ATOM | 2995 | CB | ASN | B | 64 | 34.065 | 94.478 | −10.203 | 1.00 | 32.39 |
| ATOM | 2996 | CG | ASN | B | 64 | 33.188 | 95.722 | −10.099 | 1.00 | 34.17 |
| ATOM | 2997 | OD1 | ASN | B | 64 | 32.086 | 95.689 | −9.543 | 1.00 | 35.90 |
| ATOM | 2998 | ND2 | ASN | B | 64 | 33.674 | 96.828 | −10.646 | 1.00 | 34.72 |
| ATOM | 2999 | C | ASN | B | 64 | 34.282 | 94.489 | −7.681 | 1.00 | 30.61 |
| ATOM | 3000 | O | ASN | B | 64 | 33.497 | 94.413 | −6.738 | 1.00 | 31.75 |
| ATOM | 3001 | N | GLY | B | 65 | 35.328 | 95.309 | −7.694 | 1.00 | 29.88 |
| ATOM | 3002 | CA | GLY | B | 65 | 35.617 | 96.185 | −6.578 | 1.00 | 28.45 |
| ATOM | 3003 | C | GLY | B | 65 | 36.936 | 96.903 | −6.745 | 1.00 | 28.07 |
| ATOM | 3004 | O | GLY | B | 65 | 37.622 | 96.729 | −7.748 | 1.00 | 29.15 |
| ATOM | 3005 | N | SER | B | 66 | 37.287 | 97.708 | −5.755 | 1.00 | 27.55 |
| ATOM | 3006 | CA | SER | B | 66 | 38.531 | 98.481 | −5.747 | 1.00 | 26.86 |
| ATOM | 3007 | CB | SER | B | 66 | 38.573 | 99.363 | −4.498 | 1.00 | 31.61 |
| ATOM | 3008 | OG | SER | B | 66 | 38.374 | 98.581 | −3.325 | 1.00 | 36.30 |
| ATOM | 3009 | C | SER | B | 66 | 39.763 | 97.584 | −5.766 | 1.00 | 26.13 |
| ATOM | 3010 | O | SER | B | 66 | 40.881 | 98.061 | −5.979 | 1.00 | 26.30 |
| ATOM | 3011 | N | PHE | B | 67 | 39.551 | 96.290 | −5.538 | 1.00 | 24.20 |
| ATOM | 3012 | CA | PHE | B | 67 | 40.625 | 95.303 | −5.526 | 1.00 | 23.70 |
| ATOM | 3013 | CB | PHE | B | 67 | 40.308 | 94.201 | −4.519 | 1.00 | 19.77 |
| ATOM | 3014 | CG | PHE | B | 67 | 39.001 | 93.542 | −4.787 | 1.00 | 21.78 |
| ATOM | 3015 | CD1 | PHE | B | 67 | 37.892 | 93.860 | −4.036 | 1.00 | 19.62 |
| ATOM | 3016 | CD2 | PHE | B | 67 | 38.875 | 92.622 | −5.816 | 1.00 | 22.71 |
| ATOM | 3017 | CE1 | PHE | B | 67 | 36.676 | 93.276 | −4.297 | 1.00 | 20.09 |
| ATOM | 3018 | CE2 | PHE | B | 67 | 37.662 | 92.029 | −6.084 | 1.00 | 21.51 |
| ATOM | 3019 | CZ | PHE | B | 67 | 36.558 | 92.359 | −5.322 | 1.00 | 23.29 |
| ATOM | 3020 | C | PHE | B | 67 | 40.827 | 94.686 | −6.917 | 1.00 | 25.04 |
| ATOM | 3021 | O | PHE | B | 67 | 41.933 | 94.267 | −7.250 | 1.00 | 25.12 |
| ATOM | 3022 | N | GLY | B | 68 | 39.749 | 94.643 | −7.724 | 1.00 | 27.00 |
| ATOM | 3023 | CA | GLY | B | 68 | 39.842 | 94.097 | −9.070 | 1.00 | 25.19 |
| ATOM | 3024 | C | GLY | B | 68 | 38.965 | 92.885 | −9.349 | 1.00 | 25.52 |
| ATOM | 3025 | O | GLY | B | 68 | 39.227 | 91.803 | −8.847 | 1.00 | 29.19 |
| ATOM | 3026 | N | VAL | B | 69 | 37.969 | 93.092 | −10.211 | 1.00 | 25.48 |
| ATOM | 3027 | CA | VAL | B | 69 | 36.987 | 92.107 | −10.735 | 1.00 | 23.30 |
| ATOM | 3028 | CB | VAL | B | 69 | 37.163 | 91.960 | −12.266 | 1.00 | 24.41 |
| ATOM | 3029 | CG1 | VAL | B | 69 | 36.106 | 91.032 | −12.863 | 1.00 | 21.84 |
| ATOM | 3030 | CG2 | VAL | B | 69 | 37.125 | 93.329 | −12.934 | 1.00 | 25.19 |
| ATOM | 3031 | C | VAL | B | 69 | 36.976 | 90.698 | −10.128 | 1.00 | 22.00 |
| ATOM | 3032 | O | VAL | B | 69 | 38.003 | 90.099 | −9.872 | 1.00 | 21.55 |
| ATOM | 3033 | N | VAL | B | 70 | 35.768 | 90.154 | −9.986 | 1.00 | 22.14 |
| ATOM | 3034 | CA | VAL | B | 70 | 35.551 | 88.799 | −9.489 | 1.00 | 24.12 |
| ATOM | 3035 | CB | VAL | B | 70 | 34.625 | 88.783 | −8.250 | 1.00 | 24.16 |
| ATOM | 3036 | CG1 | VAL | B | 70 | 34.275 | 87.355 | −7.860 | 1.00 | 22.28 |
| ATOM | 3037 | CG2 | VAL | B | 70 | 35.280 | 89.508 | −7.087 | 1.00 | 24.57 |
| ATOM | 3038 | C | VAL | B | 70 | 34.906 | 87.961 | −10.582 | 1.00 | 26.12 |
| ATOM | 3039 | O | VAL | B | 70 | 33.887 | 88.354 | −11.143 | 1.00 | 27.62 |
| ATOM | 3040 | N | TYR | B | 71 | 35.508 | 86.821 | −10.891 | 1.00 | 28.54 |
| ATOM | 3041 | CA | TYR | B | 71 | 34.993 | 85.953 | −11.941 | 1.00 | 30.91 |
| ATOM | 3042 | CB | TYR | B | 71 | 36.127 | 85.503 | −12.868 | 1.00 | 32.02 |
| ATOM | 3043 | CG | TYR | B | 71 | 36.962 | 86.617 | −13.438 | 1.00 | 33.80 |
| ATOM | 3044 | CD1 | TYR | B | 71 | 36.682 | 87.152 | −14.692 | 1.00 | 35.50 |
| ATOM | 3045 | CE1 | TYR | B | 71 | 37.458 | 88.166 | −15.224 | 1.00 | 35.76 |
| ATOM | 3046 | CD2 | TYR | B | 71 | 38.040 | 87.128 | −12.731 | 1.00 | 33.91 |
| ATOM | 3047 | CE2 | TYR | B | 71 | 38.820 | 88.144 | −13.251 | 1.00 | 36.55 |
| ATOM | 3048 | CZ | TYR | B | 71 | 38.526 | 88.659 | −14.499 | 1.00 | 36.56 |
| ATOM | 3049 | OH | TYR | B | 71 | 39.302 | 89.670 | −15.021 | 1.00 | 36.97 |
| ATOM | 3050 | C | TYR | B | 71 | 34.308 | 84.712 | −11.409 | 1.00 | 31.87 |
| ATOM | 3051 | O | TYR | B | 71 | 34.533 | 84.288 | −10.281 | 1.00 | 33.24 |
| ATOM | 3052 | N | GLN | B | 72 | 33.521 | 84.102 | −12.278 | 1.00 | 32.22 |
| ATOM | 3053 | CA | GLN | B | 72 | 32.840 | 82.853 | −11.989 | 1.00 | 33.30 |
| ATOM | 3054 | CB | GLN | B | 72 | 31.331 | 82.971 | −12.182 | 1.00 | 34.02 |
| ATOM | 3055 | CG | GLN | B | 72 | 30.578 | 81.698 | −11.824 | 1.00 | 36.90 |
| ATOM | 3056 | CD | GLN | B | 72 | 29.132 | 81.713 | −12.278 | 1.00 | 39.86 |
| ATOM | 3057 | OE1 | GLN | B | 72 | 28.226 | 81.366 | −11.518 | 1.00 | 40.64 |
| ATOM | 3058 | NE2 | GLN | B | 72 | 28.907 | 82.110 | −13.526 | 1.00 | 41.43 |
| ATOM | 3059 | C | GLN | B | 72 | 33.414 | 81.805 | −12.935 | 1.00 | 34.05 |
| ATOM | 3060 | O | GLN | B | 72 | 34.009 | 82.152 | −13.957 | 1.00 | 35.56 |
| ATOM | 3061 | N | ALA | B | 73 | 33.268 | 80.541 | −12.600 | 1.00 | 33.40 |
| ATOM | 3062 | CA | ALA | B | 73 | 33.809 | 79.497 | −13.447 | 1.00 | 33.76 |
| ATOM | 3063 | CB | ALA | B | 73 | 35.322 | 79.590 | −13.514 | 1.00 | 33.45 |
| ATOM | 3064 | C | ALA | B | 73 | 33.376 | 78.131 | −12.976 | 1.00 | 34.42 |
| ATOM | 3065 | O | ALA | B | 73 | 32.735 | 77.995 | −11.940 | 1.00 | 35.38 |
| ATOM | 3066 | N | LYS | B | 74 | 33.720 | 77.123 | −13.758 | 1.00 | 35.98 |
| ATOM | 3067 | CA | LYS | B | 74 | 33.357 | 75.754 | −13.445 | 1.00 | 36.46 |
| ATOM | 3068 | CB | LYS | B | 74 | 32.283 | 75.279 | −14.435 | 1.00 | 38.65 |
| ATOM | 3069 | CG | LYS | B | 74 | 31.862 | 73.821 | −14.272 | 1.00 | 41.25 |
| ATOM | 3070 | CD | LYS | B | 74 | 30.968 | 73.360 | −15.422 | 1.00 | 42.84 |
| ATOM | 3071 | CE | LYS | B | 74 | 29.534 | 73.854 | −15.259 | 1.00 | 45.68 |
| ATOM | 3072 | NZ | LYS | B | 74 | 28.787 | 73.877 | −16.561 | 1.00 | 49.00 |
| ATOM | 3073 | C | LYS | B | 74 | 34.579 | 74.854 | −13.505 | 1.00 | 36.82 |
| ATOM | 3074 | O | LYS | B | 74 | 35.262 | 74.798 | −14.531 | 1.00 | 36.80 |
| ATOM | 3075 | N | LEU | B | 75 | 34.841 | 74.141 | −12.406 | 1.00 | 36.09 |
| ATOM | 3076 | CA | LEU | B | 75 | 35.970 | 73.220 | −12.354 | 1.00 | 36.88 |
| ATOM | 3077 | CB | LEU | B | 75 | 36.156 | 72.640 | −10.944 | 1.00 | 34.57 |
| ATOM | 3078 | CG | LEU | B | 75 | 36.267 | 73.649 | −9.794 | 1.00 | 33.76 |
| ATOM | 3079 | CD1 | LEU | B | 75 | 36.456 | 72.925 | −8.468 | 1.00 | 32.85 |
| ATOM | 3080 | CD2 | LEU | B | 75 | 37.403 | 74.635 | −10.039 | 1.00 | 30.51 |
| ATOM | 3081 | C | LEU | B | 75 | 35.725 | 72.102 | −13.360 | 1.00 | 38.27 |
| ATOM | 3082 | O | LEU | B | 75 | 34.668 | 71.473 | −13.355 | 1.00 | 36.48 |
| ATOM | 3083 | N | CYS | B | 76 | 36.680 | 71.891 | −14.253 | 1.00 | 40.83 |
| ATOM | 3084 | CA | CYS | B | 76 | 36.536 | 70.879 | −15.292 | 1.00 | 43.19 |
| ATOM | 3085 | CB | CYS | B | 76 | 37.699 | 70.935 | −16.276 | 1.00 | 42.51 |
| ATOM | 3086 | SG | CYS | B | 76 | 37.745 | 72.478 | −17.226 | 1.00 | 42.48 |
| ATOM | 3087 | C | CYS | B | 76 | 36.301 | 69.480 | −14.730 | 1.00 | 45.58 |
| ATOM | 3088 | O | CYS | B | 76 | 35.391 | 68.777 | −15.177 | 1.00 | 46.73 |
| ATOM | 3089 | N | ASP | B | 77 | 37.101 | 69.088 | −13.735 | 1.00 | 48.25 |
| ATOM | 3090 | CA | ASP | B | 77 | 36.957 | 67.775 | −13.097 | 1.00 | 49.99 |
| ATOM | 3091 | CB | ASP | B | 77 | 38.021 | 67.589 | −12.004 | 1.00 | 52.61 |
| ATOM | 3092 | CG | ASP | B | 77 | 38.090 | 68.757 | −11.026 | 1.00 | 55.04 |
| ATOM | 3093 | OD1 | ASP | B | 77 | 37.638 | 68.587 | −9.868 | 1.00 | 56.33 |
| ATOM | 3094 | OD2 | ASP | B | 77 | 38.597 | 69.840 | −11.418 | 1.00 | 55.51 |
| ATOM | 3095 | C | ASP | B | 77 | 35.550 | 67.633 | −12.513 | 1.00 | 50.49 |
| ATOM | 3096 | O | ASP | B | 77 | 34.689 | 66.968 | −13.092 | 1.00 | 51.15 |
| ATOM | 3097 | N | SER | B | 78 | 35.316 | 68.306 | −11.389 | 1.00 | 49.73 |
| ATOM | 3098 | CA | SER | B | 78 | 34.013 | 68.317 | −10.738 | 1.00 | 49.21 |
| ATOM | 3099 | CB | SER | B | 78 | 34.181 | 68.391 | −9.218 | 1.00 | 49.35 |
| ATOM | 3100 | OG | SER | B | 78 | 35.081 | 69.425 | −8.853 | 1.00 | 48.54 |
| ATOM | 3101 | C | SER | B | 78 | 33.268 | 69.546 | −11.231 | 1.00 | 48.71 |
| ATOM | 3102 | O | SER | B | 78 | 33.792 | 70.651 | −11.133 | 1.00 | 50.88 |
| ATOM | 3103 | N | GLY | B | 79 | 32.077 | 69.354 | −11.794 | 1.00 | 46.72 |
| ATOM | 3104 | CA | GLY | B | 79 | 31.309 | 70.471 | −12.337 | 1.00 | 44.51 |
| ATOM | 3105 | C | GLY | B | 79 | 31.000 | 71.572 | −11.340 | 1.00 | 43.20 |
| ATOM | 3106 | O | GLY | B | 79 | 30.140 | 72.418 | −11.595 | 1.00 | 43.40 |
| ATOM | 3107 | N | GLU | B | 80 | 31.691 | 71.563 | −10.207 | 1.00 | 42.11 |
| ATOM | 3108 | CA | GLU | B | 80 | 31.487 | 72.557 | −9.166 | 1.00 | 41.04 |
| ATOM | 3109 | CB | GLU | B | 80 | 32.315 | 72.217 | −7.922 | 1.00 | 41.90 |
| ATOM | 3110 | CG | GLU | B | 80 | 31.699 | 71.145 | −7.036 | 1.00 | 45.04 |
| ATOM | 3111 | CD | GLU | B | 80 | 32.561 | 70.813 | −5.831 | 1.00 | 47.24 |
| ATOM | 3112 | OE1 | GLU | B | 80 | 33.698 | 70.335 | −6.029 | 1.00 | 48.62 |
| ATOM | 3113 | OE2 | GLU | B | 80 | 32.107 | 71.049 | −4.688 | 1.00 | 48.59 |
| ATOM | 3114 | C | GLU | B | 80 | 31.800 | 73.970 | −9.649 | 1.00 | 39.68 |
| ATOM | 3115 | O | GLU | B | 80 | 32.860 | 74.236 | −10.218 | 1.00 | 38.76 |
| ATOM | 3116 | N | LEU | B | 81 | 30.873 | 74.875 | −9.385 | 1.00 | 38.53 |
| ATOM | 3117 | CA | LEU | B | 81 | 31.030 | 76.278 | −9.742 | 1.00 | 36.65 |
| ATOM | 3118 | CB | LEU | B | 81 | 29.657 | 76.960 | −9.771 | 1.00 | 37.93 |
| ATOM | 3119 | CG | LEU | B | 81 | 28.814 | 76.770 | −11.032 | 1.00 | 38.35 |
| ATOM | 3120 | CD1 | LEU | B | 81 | 27.597 | 75.903 | −10.737 | 1.00 | 38.97 |
| ATOM | 3121 | CD2 | LEU | B | 81 | 28.388 | 78.122 | −11.583 | 1.00 | 39.14 |
| ATOM | 3122 | C | LEU | B | 81 | 31.893 | 76.973 | −8.693 | 1.00 | 35.35 |
| ATOM | 3123 | O | LEU | B | 81 | 31.798 | 76.669 | −7.502 | 1.00 | 35.32 |
| ATOM | 3124 | N | VAL | B | 82 | 32.714 | 77.914 | −9.127 | 1.00 | 32.39 |
| ATOM | 3125 | CA | VAL | B | 82 | 33.556 | 78.646 | −8.206 | 1.00 | 29.03 |
| ATOM | 3126 | CB | VAL | B | 82 | 34.971 | 78.024 | −8.085 | 1.00 | 30.73 |
| ATOM | 3127 | CG1 | VAL | B | 82 | 34.905 | 76.603 | −7.535 | 1.00 | 30.66 |
| ATOM | 3128 | CG2 | VAL | B | 82 | 35.705 | 78.052 | −9.421 | 1.00 | 32.95 |
| ATOM | 3129 | C | VAL | B | 82 | 33.699 | 80.101 | −8.626 | 1.00 | 28.50 |
| ATOM | 3130 | O | VAL | B | 82 | 33.447 | 80.469 | −9.783 | 1.00 | 27.83 |
| ATOM | 3131 | N | ALA | B | 83 | 34.159 | 80.915 | −7.693 | 1.00 | 24.36 |
| ATOM | 3132 | CA | ALA | B | 83 | 34.415 | 82.306 | −7.958 | 1.00 | 21.31 |
| ATOM | 3133 | CB | ALA | B | 83 | 33.745 | 83.201 | −6.929 | 1.00 | 22.68 |
| ATOM | 3134 | C | ALA | B | 83 | 35.915 | 82.501 | −7.956 | 1.00 | 21.78 |
| ATOM | 3135 | O | ALA | B | 83 | 36.647 | 81.684 | −7.407 | 1.00 | 26.14 |
| ATOM | 3136 | N | ILE | B | 84 | 36.377 | 83.558 | −8.578 | 1.00 | 21.94 |
| ATOM | 3137 | CA | ILE | B | 84 | 37.798 | 83.847 | −8.643 | 1.00 | 21.77 |
| ATOM | 3138 | CB | ILE | B | 84 | 38.433 | 83.419 | −9.987 | 1.00 | 22.25 |
| ATOM | 3139 | CG2 | ILE | B | 84 | 39.949 | 83.531 | −9.910 | 1.00 | 18.77 |
| ATOM | 3140 | CG1 | ILE | B | 84 | 38.002 | 81.993 | −10.360 | 1.00 | 18.57 |
| ATOM | 3141 | CD1 | ILE | B | 84 | 39.021 | 81.226 | −11.176 | 1.00 | 21.85 |
| ATOM | 3142 | C | ILE | B | 84 | 37.973 | 85.323 | −8.450 | 1.00 | 24.02 |
| ATOM | 3143 | O | ILE | B | 84 | 37.587 | 86.101 | −9.317 | 1.00 | 25.19 |
| ATOM | 3144 | N | LYS | B | 85 | 38.502 | 85.699 | −7.283 | 1.00 | 25.57 |
| ATOM | 3145 | CA | LYS | B | 85 | 38.682 | 87.102 | −6.923 | 1.00 | 26.87 |
| ATOM | 3146 | CB | LYS | B | 85 | 39.024 | 87.238 | −5.433 | 1.00 | 27.80 |
| ATOM | 3147 | CG | LYS | B | 85 | 38.917 | 88.654 | −4.919 | 1.00 | 27.48 |
| ATOM | 3148 | CD | LYS | B | 85 | 38.186 | 88.713 | −3.585 | 1.00 | 28.88 |
| ATOM | 3149 | CE | LYS | B | 85 | 38.215 | 90.121 | −3.004 | 1.00 | 29.25 |
| ATOM | 3150 | NZ | LYS | B | 85 | 37.788 | 90.150 | −1.571 | 1.00 | 31.82 |
| ATOM | 3151 | C | LYS | B | 85 | 39.732 | 87.817 | −7.777 | 1.00 | 28.36 |
| ATOM | 3152 | O | LYS | B | 85 | 39.404 | 88.764 | −8.475 | 1.00 | 32.62 |
| ATOM | 3153 | N | LYS | B | 86 | 40.982 | 87.406 | −7.725 | 1.00 | 27.30 |
| ATOM | 3154 | CA | LYS | B | 86 | 42.012 | 88.079 | −8.524 | 1.00 | 28.47 |
| ATOM | 3155 | CB | LYS | B | 86 | 41.647 | 88.040 | −10.024 | 1.00 | 29.75 |
| ATOM | 3156 | CG | LYS | B | 86 | 42.626 | 88.785 | −10.918 | 1.00 | 30.30 |
| ATOM | 3157 | CD | LYS | B | 86 | 42.448 | 88.399 | −12.385 | 1.00 | 31.97 |
| ATOM | 3158 | CE | LYS | B | 86 | 43.646 | 88.817 | −13.229 | 1.00 | 32.33 |
| ATOM | 3159 | NZ | LYS | B | 86 | 43.528 | 90.223 | −13.718 | 1.00 | 32.44 |
| ATOM | 3160 | C | LYS | B | 86 | 42.191 | 89.526 | −8.080 | 1.00 | 28.16 |
| ATOM | 3161 | O | LYS | B | 86 | 41.398 | 90.390 | −8.426 | 1.00 | 27.79 |
| ATOM | 3162 | N | VAL | B | 87 | 43.237 | 89.787 | −7.310 | 1.00 | 30.21 |
| ATOM | 3163 | CA | VAL | B | 87 | 43.505 | 91.136 | −6.818 | 1.00 | 31.57 |
| ATOM | 3164 | CB | VAL | B | 87 | 43.348 | 91.245 | −5.275 | 1.00 | 30.87 |
| ATOM | 3165 | CG1 | VAL | B | 87 | 42.229 | 90.345 | −4.773 | 1.00 | 28.72 |
| ATOM | 3166 | CG2 | VAL | B | 87 | 44.652 | 90.928 | −4.564 | 1.00 | 30.54 |
| ATOM | 3167 | C | VAL | B | 87 | 44.888 | 91.624 | −7.228 | 1.00 | 32.63 |
| ATOM | 3168 | O | VAL | B | 87 | 45.834 | 90.847 | −7.301 | 1.00 | 32.63 |
| ATOM | 3169 | N | LEU | B | 88 | 44.994 | 92.923 | −7.479 | 1.00 | 36.02 |
| ATOM | 3170 | CA | LEU | B | 88 | 46.258 | 93.530 | −7.864 | 1.00 | 38.65 |
| ATOM | 3171 | CB | LEU | B | 88 | 46.027 | 94.939 | −8.428 | 1.00 | 38.60 |
| ATOM | 3172 | CG | LEU | B | 88 | 46.980 | 95.389 | −9.542 | 1.00 | 39.38 |
| ATOM | 3173 | CD1 | LEU | B | 88 | 46.418 | 96.600 | −10.274 | 1.00 | 39.21 |
| ATOM | 3174 | CD2 | LEU | B | 88 | 48.361 | 95.697 | −8.981 | 1.00 | 38.72 |
| ATOM | 3175 | C | LEU | B | 88 | 47.170 | 93.594 | −6.654 | 1.00 | 41.80 |
| ATOM | 3176 | O | LEU | B | 88 | 46.864 | 94.277 | −5.672 | 1.00 | 43.27 |
| ATOM | 3177 | N | GLN | B | 89 | 48.277 | 92.867 | −6.715 | 1.00 | 44.15 |
| ATOM | 3178 | CA | GLN | B | 89 | 49.223 | 92.830 | −5.608 | 1.00 | 46.43 |
| ATOM | 3179 | CB | GLN | B | 89 | 49.313 | 91.407 | −5.054 | 1.00 | 46.20 |
| ATOM | 3180 | CG | GLN | B | 89 | 48.297 | 91.098 | −3.973 | 1.00 | 47.54 |
| ATOM | 3181 | CD | GLN | B | 89 | 48.617 | 91.789 | −2.668 | 1.00 | 47.37 |
| ATOM | 3182 | OE1 | GLN | B | 89 | 47.905 | 92.700 | −2.245 | 1.00 | 49.60 |
| ATOM | 3183 | NE2 | GLN | B | 89 | 49.695 | 91.361 | −2.022 | 1.00 | 49.76 |
| ATOM | 3184 | C | GLN | B | 89 | 50.618 | 93.309 | −6.009 | 1.00 | 48.98 |
| ATOM | 3185 | O | GLN | B | 89 | 50.911 | 93.544 | −7.187 | 1.00 | 49.03 |
| ATOM | 3186 | N | ASP | B | 90 | 51.481 | 93.419 | −5.008 | 1.00 | 50.77 |
| ATOM | 3187 | CA | ASP | B | 90 | 52.864 | 93.827 | −5.202 | 1.00 | 52.86 |
| ATOM | 3188 | CB | ASP | B | 90 | 53.067 | 95.278 | −4.755 | 1.00 | 52.56 |
| ATOM | 3189 | CG | ASP | B | 90 | 54.310 | 95.921 | −5.352 | 1.00 | 52.43 |
| ATOM | 3190 | OD1 | ASP | B | 90 | 54.173 | 96.967 | −6.024 | 1.00 | 51.92 |
| ATOM | 3191 | OD2 | ASP | B | 90 | 55.421 | 95.385 | −5.141 | 1.00 | 52.38 |
| ATOM | 3192 | C | ASP | B | 90 | 53.777 | 92.891 | −4.409 | 1.00 | 54.65 |
| ATOM | 3193 | O | ASP | B | 90 | 53.481 | 92.563 | −3.252 | 1.00 | 55.24 |
| ATOM | 3194 | N | LYS | B | 91 | 54.873 | 92.451 | −5.039 | 1.00 | 55.69 |
| ATOM | 3195 | CA | LYS | B | 91 | 55.824 | 91.507 | −4.429 | 1.00 | 56.44 |
| ATOM | 3196 | CB | LYS | B | 91 | 57.142 | 91.480 | −5.177 | 1.00 | 56.74 |
| ATOM | 3197 | CG | LYS | B | 91 | 57.101 | 90.724 | −6.482 | 1.00 | 56.89 |
| ATOM | 3198 | CD | LYS | B | 91 | 56.988 | 91.646 | −7.701 | 1.00 | 56.85 |
| ATOM | 3199 | CE | LYS | B | 91 | 58.103 | 91.365 | −8.705 | 1.00 | 56.60 |
| ATOM | 3200 | NZ | LYS | B | 91 | 59.228 | 92.329 | −8.574 | 1.00 | 54.94 |
| ATOM | 3201 | C | LYS | B | 91 | 56.118 | 91.932 | −3.003 | 1.00 | 57.84 |
| ATOM | 3202 | O | LYS | B | 91 | 56.174 | 91.130 | −2.073 | 1.00 | 57.47 |
| ATOM | 3203 | N | ARG | B | 92 | 56.293 | 93.242 | −2.898 | 1.00 | 59.51 |
| ATOM | 3204 | CA | ARG | B | 92 | 56.440 | 93.812 | −1.612 | 1.00 | 60.32 |
| ATOM | 3205 | CB | ARG | B | 92 | 56.567 | 95.344 | −1.628 | 1.00 | 61.19 |
| ATOM | 3206 | CG | ARG | B | 92 | 55.223 | 96.051 | −1.807 | 1.00 | 62.74 |
| ATOM | 3207 | CD | ARG | B | 92 | 55.091 | 97.310 | −0.936 | 1.00 | 64.16 |
| ATOM | 3208 | NE | ARG | B | 92 | 53.734 | 97.452 | −0.383 | 1.00 | 64.89 |
| ATOM | 3209 | CZ | ARG | B | 92 | 52.690 | 97.943 | −1.040 | 1.00 | 65.15 |
| ATOM | 3210 | NH1 | ARG | B | 92 | 52.815 | 98.353 | −2.303 | 1.00 | 64.76 |
| ATOM | 3211 | NH2 | ARG | B | 92 | 51.510 | 98.013 | −0.431 | 1.00 | 65.43 |
| ATOM | 3212 | C | ARG | B | 92 | 55.257 | 93.328 | −0.871 | 1.00 | 60.27 |
| ATOM | 3213 | O | ARG | B | 92 | 54.114 | 93.626 | −1.167 | 1.00 | 60.72 |
| ATOM | 3214 | N | PHE | B | 93 | 55.628 | 92.551 | 0.093 | 1.00 | 59.54 |
| ATOM | 3215 | CA | PHE | B | 93 | 54.728 | 92.058 | 1.098 | 1.00 | 58.24 |
| ATOM | 3216 | CB | PHE | B | 93 | 53.931 | 93.238 | 1.611 | 1.00 | 59.80 |
| ATOM | 3217 | CG | PHE | B | 93 | 53.712 | 92.969 | 3.023 | 1.00 | 60.86 |
| ATOM | 3218 | CD1 | PHE | B | 93 | 54.604 | 93.373 | 4.005 | 1.00 | 61.43 |
| ATOM | 3219 | CD2 | PHE | B | 93 | 52.576 | 92.292 | 3.388 | 1.00 | 61.79 |
| ATOM | 3220 | CE1 | PHE | B | 93 | 54.375 | 93.046 | 5.329 | 1.00 | 61.52 |
| ATOM | 3221 | CE2 | PHE | B | 93 | 52.334 | 91.959 | 4.705 | 1.00 | 61.96 |
| ATOM | 3222 | CZ | PHE | B | 93 | 53.237 | 92.338 | 5.680 | 1.00 | 62.16 |
| ATOM | 3223 | C | PHE | B | 93 | 53.820 | 90.910 | 0.699 | 1.00 | 56.34 |
| ATOM | 3224 | O | PHE | B | 93 | 53.778 | 90.473 | −0.455 | 1.00 | 57.26 |
| ATOM | 3225 | N | LYS | B | 94 | 53.119 | 90.395 | 1.720 | 1.00 | 52.33 |
| ATOM | 3226 | CA | LYS | B | 94 | 52.161 | 89.300 | 1.568 | 1.00 | 48.31 |
| ATOM | 3227 | CB | LYS | B | 94 | 52.287 | 88.270 | 2.684 | 1.00 | 49.90 |
| ATOM | 3228 | CG | LYS | B | 94 | 53.621 | 87.546 | 2.734 | 1.00 | 52.02 |
| ATOM | 3229 | CD | LYS | B | 94 | 53.888 | 86.960 | 4.100 | 1.00 | 53.08 |
| ATOM | 3230 | CE | LYS | B | 94 | 53.772 | 88.029 | 5.182 | 1.00 | 54.32 |
| ATOM | 3231 | NZ | LYS | B | 94 | 54.367 | 87.569 | 6.472 | 1.00 | 55.37 |
| ATOM | 3232 | C | LYS | B | 94 | 50.758 | 89.872 | 1.626 | 1.00 | 44.39 |
| ATOM | 3233 | O | LYS | B | 94 | 50.553 | 90.937 | 2.204 | 1.00 | 45.14 |
| ATOM | 3234 | N | ASN | B | 95 | 49.794 | 89.177 | 1.034 | 1.00 | 39.77 |
| ATOM | 3235 | CA | ASN | B | 95 | 48.405 | 89.632 | 1.033 | 1.00 | 35.63 |
| ATOM | 3236 | CB | ASN | B | 95 | 47.633 | 88.938 | −0.097 | 1.00 | 32.69 |
| ATOM | 3237 | CG | ASN | B | 95 | 46.296 | 89.579 | −0.405 | 1.00 | 30.84 |
| ATOM | 3238 | OD1 | ASN | B | 95 | 46.226 | 90.620 | −1.056 | 1.00 | 31.82 |
| ATOM | 3239 | ND2 | ASN | B | 95 | 45.223 | 88.948 | 0.074 | 1.00 | 30.54 |
| ATOM | 3240 | C | ASN | B | 95 | 47.767 | 89.300 | 2.384 | 1.00 | 32.38 |
| ATOM | 3241 | O | ASN | B | 95 | 47.910 | 88.186 | 2.870 | 1.00 | 32.37 |
| ATOM | 3242 | N | ARG | B | 96 | 47.078 | 90.267 | 2.985 | 1.00 | 31.13 |
| ATOM | 3243 | CA | ARG | B | 96 | 46.436 | 90.058 | 4.287 | 1.00 | 27.36 |
| ATOM | 3244 | CB | ARG | B | 96 | 45.985 | 91.396 | 4.889 | 1.00 | 27.69 |
| ATOM | 3245 | CG | ARG | B | 96 | 45.479 | 91.288 | 6.331 | 1.00 | 28.43 |
| ATOM | 3246 | CD | ARG | B | 96 | 46.149 | 92.303 | 7.239 | 1.00 | 29.78 |
| ATOM | 3247 | NE | ARG | B | 96 | 45.441 | 93.580 | 7.248 | 1.00 | 30.41 |
| ATOM | 3248 | CZ | ARG | B | 96 | 45.959 | 94.720 | 7.709 | 1.00 | 30.73 |
| ATOM | 3249 | NH1 | ARG | B | 96 | 47.204 | 94.760 | 8.188 | 1.00 | 32.67 |
| ATOM | 3250 | NH2 | ARG | B | 96 | 45.229 | 95.827 | 7.690 | 1.00 | 31.28 |
| ATOM | 3251 | C | ARG | B | 96 | 45.248 | 89.116 | 4.158 | 1.00 | 26.30 |
| ATOM | 3252 | O | ARG | B | 96 | 45.040 | 88.237 | 4.995 | 1.00 | 24.70 |
| ATOM | 3253 | N | GLU | B | 97 | 44.466 | 89.298 | 3.098 | 1.00 | 24.76 |
| ATOM | 3254 | CA | GLU | B | 97 | 43.303 | 88.458 | 2.867 | 1.00 | 22.51 |
| ATOM | 3255 | CB | GLU | B | 97 | 42.435 | 89.003 | 1.733 | 1.00 | 24.93 |
| ATOM | 3256 | CG | GLU | B | 97 | 41.267 | 88.104 | 1.334 | 1.00 | 22.80 |
| ATOM | 3257 | CD | GLU | B | 97 | 40.263 | 88.812 | 0.447 | 1.00 | 21.64 |
| ATOM | 3258 | OE1 | GLU | B | 97 | 39.079 | 88.414 | 0.461 | 1.00 | 25.04 |
| ATOM | 3259 | OE2 | GLU | B | 97 | 40.655 | 89.772 | −0.253 | 1.00 | 21.61 |
| ATOM | 3260 | C | GLU | B | 97 | 43.686 | 87.009 | 2.621 | 1.00 | 21.52 |
| ATOM | 3261 | O | GLU | B | 97 | 42.930 | 86.109 | 2.985 | 1.00 | 24.82 |
| ATOM | 3262 | N | LEU | B | 98 | 44.855 | 86.778 | 2.014 | 1.00 | 19.37 |
| ATOM | 3263 | CA | LEU | B | 98 | 45.308 | 85.413 | 1.740 | 1.00 | 18.57 |
| ATOM | 3264 | CB | LEU | B | 98 | 46.387 | 85.371 | 0.664 | 1.00 | 16.63 |
| ATOM | 3265 | CG | LEU | B | 98 | 46.977 | 83.985 | 0.367 | 1.00 | 16.92 |
| ATOM | 3266 | CD1 | LEU | B | 98 | 45.897 | 83.012 | −0.077 | 1.00 | 13.54 |
| ATOM | 3267 | CD2 | LEU | B | 98 | 48.094 | 84.072 | −0.672 | 1.00 | 18.79 |
| ATOM | 3268 | C | LEU | B | 98 | 45.811 | 84.700 | 3.000 | 1.00 | 18.68 |
| ATOM | 3269 | O | LEU | B | 98 | 45.533 | 83.525 | 3.194 | 1.00 | 15.55 |
| ATOM | 3270 | N | GLN | B | 99 | 46.560 | 85.411 | 3.835 | 1.00 | 19.69 |
| ATOM | 3271 | CA | GLN | B | 99 | 47.084 | 84.812 | 5.058 | 1.00 | 23.79 |
| ATOM | 3272 | CB | GLN | B | 99 | 48.052 | 85.757 | 5.787 | 1.00 | 27.75 |
| ATOM | 3273 | CG | GLN | B | 99 | 49.373 | 85.988 | 5.059 | 1.00 | 33.53 |
| ATOM | 3274 | CD | GLN | B | 99 | 50.471 | 84.998 | 5.447 | 1.00 | 38.62 |
| ATOM | 3275 | OE1 | GLN | B | 99 | 51.624 | 85.384 | 5.657 | 1.00 | 39.28 |
| ATOM | 3276 | NE2 | GLN | B | 99 | 50.124 | 83.711 | 5.532 | 1.00 | 41.46 |
| ATOM | 3277 | C | GLN | B | 99 | 45.934 | 84.387 | 5.965 | 1.00 | 19.19 |
| ATOM | 3278 | O | GLN | B | 99 | 45.941 | 83.297 | 6.508 | 1.00 | 19.09 |
| ATOM | 3279 | N | ILE | B | 100 | 44.934 | 85.244 | 6.074 | 1.00 | 19.23 |
| ATOM | 3280 | CA | ILE | B | 100 | 43.747 | 84.962 | 6.876 | 1.00 | 20.77 |
| ATOM | 3281 | CB | ILE | B | 100 | 42.864 | 86.221 | 7.025 | 1.00 | 18.98 |
| ATOM | 3282 | CG2 | ILE | B | 100 | 41.433 | 85.860 | 7.381 | 1.00 | 18.23 |
| ATOM | 3283 | CG1 | ILE | B | 100 | 43.461 | 87.169 | 8.068 | 1.00 | 17.33 |
| ATOM | 3284 | CD1 | ILE | B | 100 | 42.907 | 88.570 | 8.006 | 1.00 | 12.19 |
| ATOM | 3285 | C | ILE | B | 100 | 42.921 | 83.796 | 6.314 | 1.00 | 23.82 |
| ATOM | 3286 | O | ILE | B | 100 | 42.554 | 82.875 | 7.045 | 1.00 | 24.69 |
| ATOM | 3287 | N | MET | B | 101 | 42.618 | 83.839 | 5.017 | 1.00 | 24.18 |
| ATOM | 3288 | CA | MET | B | 101 | 41.821 | 82.786 | 4.379 | 1.00 | 24.19 |
| ATOM | 3289 | CB | MET | B | 101 | 41.449 | 83.182 | 2.953 | 1.00 | 26.26 |
| ATOM | 3290 | CG | MET | B | 101 | 40.491 | 84.346 | 2.889 | 1.00 | 28.56 |
| ATOM | 3291 | SD | MET | B | 101 | 39.096 | 84.023 | 1.802 | 1.00 | 37.47 |
| ATOM | 3292 | CE | MET | B | 101 | 39.857 | 84.336 | 0.207 | 1.00 | 35.49 |
| ATOM | 3293 | C | MET | B | 101 | 42.497 | 81.418 | 4.392 | 1.00 | 22.54 |
| ATOM | 3294 | O | MET | B | 101 | 41.820 | 80.400 | 4.397 | 1.00 | 23.04 |
| ATOM | 3295 | N | ARG | B | 102 | 43.826 | 81.402 | 4.391 | 1.00 | 23.08 |
| ATOM | 3296 | CA | ARG | B | 102 | 44.595 | 80.157 | 4.393 | 1.00 | 25.61 |
| ATOM | 3297 | CB | ARG | B | 102 | 46.081 | 80.464 | 4.248 | 1.00 | 26.73 |
| ATOM | 3298 | CG | ARG | B | 102 | 46.515 | 80.714 | 2.808 | 1.00 | 30.87 |
| ATOM | 3299 | CD | ARG | B | 102 | 47.209 | 79.504 | 2.214 | 1.00 | 29.82 |
| ATOM | 3300 | NE | ARG | B | 102 | 48.581 | 79.809 | 1.828 | 1.00 | 32.54 |
| ATOM | 3301 | CZ | ARG | B | 102 | 49.489 | 78.884 | 1.525 | 1.00 | 32.00 |
| ATOM | 3302 | NH1 | ARG | B | 102 | 49.195 | 77.590 | 1.632 | 1.00 | 31.74 |
| ATOM | 3303 | NH2 | ARG | B | 102 | 50.699 | 79.255 | 1.116 | 1.00 | 34.43 |
| ATOM | 3304 | C | ARG | B | 102 | 44.382 | 79.304 | 5.653 | 1.00 | 27.37 |
| ATOM | 3305 | O | ARG | B | 102 | 44.565 | 78.085 | 5.610 | 1.00 | 26.87 |
| ATOM | 3306 | N | LYS | B | 103 | 44.025 | 79.921 | 6.783 | 1.00 | 27.22 |
| ATOM | 3307 | CA | LYS | B | 103 | 43.846 | 79.138 | 8.011 | 1.00 | 27.60 |
| ATOM | 3308 | CB | LYS | B | 103 | 44.393 | 79.896 | 9.241 | 1.00 | 27.93 |
| ATOM | 3309 | CG | LYS | B | 103 | 43.938 | 81.334 | 9.368 | 1.00 | 27.88 |
| ATOM | 3310 | CD | LYS | B | 103 | 45.024 | 82.193 | 9.987 | 1.00 | 31.30 |
| ATOM | 3311 | CE | LYS | B | 103 | 44.914 | 82.226 | 11.511 | 1.00 | 31.30 |
| ATOM | 3312 | NZ | LYS | B | 103 | 45.771 | 83.294 | 12.115 | 1.00 | 31.93 |
| ATOM | 3313 | C | LYS | B | 103 | 42.398 | 78.698 | 8.240 | 1.00 | 25.91 |
| ATOM | 3314 | O | LYS | B | 103 | 42.151 | 77.778 | 9.016 | 1.00 | 28.69 |
| ATOM | 3315 | N | LEU | B | 104 | 41.443 | 79.348 | 7.585 | 1.00 | 25.04 |
| ATOM | 3316 | CA | LEU | B | 104 | 40.035 | 79.010 | 7.775 | 1.00 | 24.60 |
| ATOM | 3317 | CB | LEU | B | 104 | 39.145 | 80.195 | 7.467 | 1.00 | 24.10 |
| ATOM | 3318 | CG | LEU | B | 104 | 39.554 | 81.518 | 8.097 | 1.00 | 25.73 |
| ATOM | 3319 | CD1 | LEU | B | 104 | 38.566 | 82.615 | 7.698 | 1.00 | 22.14 |
| ATOM | 3320 | CD2 | LEU | B | 104 | 39.648 | 81.378 | 9.620 | 1.00 | 24.52 |
| ATOM | 3321 | C | LEU | B | 104 | 39.533 | 77.774 | 7.026 | 1.00 | 26.61 |
| ATOM | 3322 | O | LEU | B | 104 | 39.899 | 77.505 | 5.872 | 1.00 | 27.07 |
| ATOM | 3323 | N | ASP | B | 105 | 38.639 | 77.061 | 7.701 | 1.00 | 23.86 |
| ATOM | 3324 | CA | ASP | B | 105 | 37.980 | 75.877 | 7.182 | 1.00 | 24.16 |
| ATOM | 3325 | CB | ASP | B | 105 | 38.895 | 74.644 | 7.229 | 1.00 | 25.34 |
| ATOM | 3326 | CG | ASP | B | 105 | 38.388 | 73.488 | 6.369 | 1.00 | 27.01 |
| ATOM | 3327 | OD1 | ASP | B | 105 | 37.178 | 73.457 | 6.045 | 1.00 | 27.72 |
| ATOM | 3328 | OD2 | ASP | B | 105 | 39.204 | 72.611 | 6.017 | 1.00 | 31.45 |
| ATOM | 3329 | C | ASP | B | 105 | 36.716 | 75.644 | 7.990 | 1.00 | 22.82 |
| ATOM | 3330 | O | ASP | B | 105 | 36.706 | 74.842 | 8.904 | 1.00 | 24.78 |
| ATOM | 3331 | N | HIS | B | 106 | 35.667 | 76.395 | 7.670 | 1.00 | 23.73 |
| ATOM | 3332 | CA | HIS | B | 106 | 34.410 | 76.312 | 8.391 | 1.00 | 24.16 |
| ATOM | 3333 | CB | HIS | B | 106 | 34.305 | 77.498 | 9.376 | 1.00 | 26.05 |
| ATOM | 3334 | CG | HIS | B | 106 | 33.192 | 77.392 | 10.378 | 1.00 | 24.58 |
| ATOM | 3335 | CD2 | HIS | B | 106 | 33.198 | 76.989 | 11.672 | 1.00 | 24.11 |
| ATOM | 3336 | ND1 | HIS | B | 106 | 31.887 | 77.723 | 10.089 | 1.00 | 24.83 |
| ATOM | 3337 | CE1 | HIS | B | 106 | 31.138 | 77.526 | 11.157 | 1.00 | 25.65 |
| ATOM | 3338 | NE2 | HIS | B | 106 | 31.912 | 77.080 | 12.129 | 1.00 | 21.29 |
| ATOM | 3339 | C | HIS | B | 106 | 33.199 | 76.260 | 7.461 | 1.00 | 23.88 |
| ATOM | 3340 | O | HIS | B | 106 | 33.111 | 77.000 | 6.475 | 1.00 | 25.00 |
| ATOM | 3341 | N | CYS | B | 107 | 32.269 | 75.374 | 7.803 | 1.00 | 24.06 |
| ATOM | 3342 | CA | CYS | B | 107 | 31.027 | 75.144 | 7.063 | 1.00 | 25.46 |
| ATOM | 3343 | CB | CYS | B | 107 | 30.192 | 74.052 | 7.744 | 1.00 | 28.48 |
| ATOM | 3344 | SG | CYS | B | 107 | 29.366 | 74.561 | 9.285 | 1.00 | 32.88 |
| ATOM | 3345 | C | CYS | B | 107 | 30.178 | 76.393 | 6.856 | 1.00 | 25.19 |
| ATOM | 3346 | O | CYS | B | 107 | 29.239 | 76.365 | 6.057 | 1.00 | 25.17 |
| ATOM | 3347 | N | ASN | B | 108 | 30.485 | 77.486 | 7.569 | 1.00 | 23.81 |
| ATOM | 3348 | CA | ASN | B | 108 | 29.705 | 78.721 | 7.424 | 1.00 | 21.66 |
| ATOM | 3349 | CB | ASN | B | 108 | 29.032 | 79.128 | 8.746 | 1.00 | 24.36 |
| ATOM | 3350 | CG | ASN | B | 108 | 27.872 | 78.227 | 9.127 | 1.00 | 24.89 |
| ATOM | 3351 | OD1 | ASN | B | 108 | 27.861 | 77.637 | 10.213 | 1.00 | 26.27 |
| ATOM | 3352 | ND2 | ASN | B | 108 | 26.883 | 78.117 | 8.242 | 1.00 | 25.27 |
| ATOM | 3353 | C | ASN | B | 108 | 30.559 | 79.865 | 6.901 | 1.00 | 20.89 |
| ATOM | 3354 | O | ASN | B | 108 | 30.171 | 81.034 | 6.993 | 1.00 | 20.76 |
| ATOM | 3355 | N | ILE | B | 109 | 31.716 | 79.520 | 6.351 | 1.00 | 19.97 |
| ATOM | 3356 | CA | ILE | B | 109 | 32.635 | 80.493 | 5.796 | 1.00 | 19.68 |
| ATOM | 3357 | CB | ILE | B | 109 | 33.913 | 80.592 | 6.646 | 1.00 | 18.06 |
| ATOM | 3358 | CG2 | ILE | B | 109 | 34.908 | 81.558 | 6.016 | 1.00 | 16.00 |
| ATOM | 3359 | CG1 | ILE | B | 109 | 33.592 | 81.001 | 8.100 | 1.00 | 18.80 |
| ATOM | 3360 | CD1 | ILE | B | 109 | 34.831 | 81.254 | 8.924 | 1.00 | 13.72 |
| ATOM | 3361 | C | ILE | B | 109 | 33.032 | 80.077 | 4.364 | 1.00 | 21.70 |
| ATOM | 3362 | O | ILE | B | 109 | 33.433 | 78.930 | 4.130 | 1.00 | 20.77 |
| ATOM | 3363 | N | VAL | B | 110 | 32.940 | 81.012 | 3.419 | 1.00 | 21.55 |
| ATOM | 3364 | CA | VAL | B | 110 | 33.320 | 80.709 | 2.041 | 1.00 | 21.63 |
| ATOM | 3365 | CB | VAL | B | 110 | 33.105 | 81.906 | 1.105 | 1.00 | 22.38 |
| ATOM | 3366 | CG1 | VAL | B | 110 | 34.312 | 82.830 | 1.105 | 1.00 | 22.92 |
| ATOM | 3367 | CG2 | VAL | B | 110 | 32.790 | 81.412 | −0.300 | 1.00 | 27.31 |
| ATOM | 3368 | C | VAL | B | 110 | 34.763 | 80.225 | 2.001 | 1.00 | 20.46 |
| ATOM | 3369 | O | VAL | B | 110 | 35.683 | 80.934 | 2.375 | 1.00 | 22.12 |
| ATOM | 3370 | N | ARG | B | 111 | 34.947 | 78.991 | 1.605 | 1.00 | 20.95 |
| ATOM | 3371 | CA | ARG | B | 111 | 36.275 | 78.392 | 1.583 | 1.00 | 23.52 |
| ATOM | 3372 | CB | ARG | B | 111 | 36.131 | 76.870 | 1.528 | 1.00 | 25.99 |
| ATOM | 3373 | CG | ARG | B | 111 | 37.383 | 76.111 | 1.916 | 1.00 | 31.22 |
| ATOM | 3374 | CD | ARG | B | 111 | 37.135 | 74.610 | 1.908 | 1.00 | 35.06 |
| ATOM | 3375 | NE | ARG | B | 111 | 38.031 | 73.906 | 0.984 | 1.00 | 39.62 |
| ATOM | 3376 | CZ | ARG | B | 111 | 37.618 | 73.149 | −0.040 | 1.00 | 41.78 |
| ATOM | 3377 | NH1 | ARG | B | 111 | 36.316 | 73.020 | −0.324 | 1.00 | 42.77 |
| ATOM | 3378 | NH2 | ARG | B | 111 | 38.518 | 72.522 | −0.790 | 1.00 | 42.63 |
| ATOM | 3379 | C | ARG | B | 111 | 37.144 | 78.871 | 0.410 | 1.00 | 24.24 |
| ATOM | 3380 | O | ARG | B | 111 | 36.639 | 79.231 | −0.652 | 1.00 | 25.26 |
| ATOM | 3381 | N | LEU | B | 112 | 38.460 | 78.809 | 0.623 | 1.00 | 22.36 |
| ATOM | 3382 | CA | LEU | B | 112 | 39.462 | 79.149 | −0.373 | 1.00 | 22.52 |
| ATOM | 3383 | CB | LEU | B | 112 | 40.598 | 79.988 | 0.223 | 1.00 | 21.19 |
| ATOM | 3384 | CG | LEU | B | 112 | 41.887 | 80.060 | −0.609 | 1.00 | 22.47 |
| ATOM | 3385 | CD1 | LEU | B | 112 | 41.740 | 81.070 | −1.738 | 1.00 | 21.76 |
| ATOM | 3386 | CD2 | LEU | B | 112 | 43.085 | 80.407 | 0.265 | 1.00 | 22.74 |
| ATOM | 3387 | C | LEU | B | 112 | 40.020 | 77.841 | −0.906 | 1.00 | 22.82 |
| ATOM | 3388 | O | LEU | B | 112 | 40.875 | 77.221 | −0.274 | 1.00 | 23.51 |
| ATOM | 3389 | N | ARG | B | 113 | 39.493 | 77.410 | −2.051 | 1.00 | 22.73 |
| ATOM | 3390 | CA | ARG | B | 113 | 39.898 | 76.151 | −2.682 | 1.00 | 21.00 |
| ATOM | 3391 | CB | ARG | B | 113 | 39.011 | 75.873 | −3.882 | 1.00 | 23.79 |
| ATOM | 3392 | CG | ARG | B | 113 | 37.537 | 75.936 | −3.562 | 1.00 | 22.88 |
| ATOM | 3393 | CD | ARG | B | 113 | 36.966 | 74.547 | −3.378 | 1.00 | 29.46 |
| ATOM | 3394 | NE | ARG | B | 113 | 35.586 | 74.464 | −3.842 | 1.00 | 31.60 |
| ATOM | 3395 | CZ | ARG | B | 113 | 35.139 | 73.536 | −4.679 | 1.00 | 30.00 |
| ATOM | 3396 | NH1 | ARG | B | 113 | 35.958 | 72.627 | −5.197 | 1.00 | 31.17 |
| ATOM | 3397 | NH2 | ARG | B | 113 | 33.865 | 73.528 | −5.005 | 1.00 | 34.17 |
| ATOM | 3398 | C | ARG | B | 113 | 41.335 | 76.192 | −3.134 | 1.00 | 18.67 |
| ATOM | 3399 | O | ARG | B | 113 | 42.112 | 75.277 | −2.876 | 1.00 | 19.77 |
| ATOM | 3400 | N | TYR | B | 114 | 41.681 | 77.250 | −3.828 | 1.00 | 19.73 |
| ATOM | 3401 | CA | TYR | B | 114 | 43.031 | 77.415 | −4.334 | 1.00 | 17.83 |
| ATOM | 3402 | CB | TYR | B | 114 | 43.184 | 76.791 | −5.736 | 1.00 | 18.92 |
| ATOM | 3403 | CG | TYR | B | 114 | 42.707 | 75.373 | −5.904 | 1.00 | 15.37 |
| ATOM | 3404 | CD1 | TYR | B | 114 | 41.386 | 75.104 | −6.166 | 1.00 | 17.28 |
| ATOM | 3405 | CE1 | TYR | B | 114 | 40.948 | 73.811 | −6.347 | 1.00 | 19.97 |
| ATOM | 3406 | CD2 | TYR | B | 114 | 43.591 | 74.312 | −5.825 | 1.00 | 17.10 |
| ATOM | 3407 | CE2 | TYR | B | 114 | 43.162 | 73.019 | −6.000 | 1.00 | 18.35 |
| ATOM | 3408 | CZ | TYR | B | 114 | 41.841 | 72.777 | −6.263 | 1.00 | 19.67 |
| ATOM | 3409 | OH | TYR | B | 114 | 41.404 | 71.490 | −6.446 | 1.00 | 26.72 |
| ATOM | 3410 | C | TYR | B | 114 | 43.344 | 78.875 | −4.473 | 1.00 | 15.22 |
| ATOM | 3411 | O | TYR | B | 114 | 42.468 | 79.707 | −4.467 | 1.00 | 17.51 |
| ATOM | 3412 | N | PHE | B | 115 | 44.597 | 79.163 | −4.665 | 1.00 | 17.48 |
| ATOM | 3413 | CA | PHE | B | 115 | 45.039 | 80.510 | −4.890 | 1.00 | 19.26 |
| ATOM | 3414 | CB | PHE | B | 115 | 45.399 | 81.242 | −3.594 | 1.00 | 18.79 |
| ATOM | 3415 | CG | PHE | B | 115 | 46.608 | 80.724 | −2.891 | 1.00 | 17.82 |
| ATOM | 3416 | CD1 | PHE | B | 115 | 47.845 | 81.278 | −3.127 | 1.00 | 17.38 |
| ATOM | 3417 | CD2 | PHE | B | 115 | 46.498 | 79.690 | −1.978 | 1.00 | 21.19 |
| ATOM | 3418 | CE1 | PHE | B | 115 | 48.966 | 80.813 | −2.468 | 1.00 | 21.69 |
| ATOM | 3419 | CE2 | PHE | B | 115 | 47.613 | 79.214 | −1.311 | 1.00 | 22.13 |
| ATOM | 3420 | CZ | PHE | B | 115 | 48.852 | 79.777 | −1.556 | 1.00 | 21.81 |
| ATOM | 3421 | C | PHE | B | 115 | 46.206 | 80.448 | −5.833 | 1.00 | 20.99 |
| ATOM | 3422 | O | PHE | B | 115 | 46.956 | 79.495 | −5.806 | 1.00 | 21.58 |
| ATOM | 3423 | N | PHE | B | 116 | 46.365 | 81.441 | −6.668 | 1.00 | 23.42 |
| ATOM | 3424 | CA | PHE | B | 116 | 47.487 | 81.423 | −7.589 | 1.00 | 23.63 |
| ATOM | 3425 | CB | PHE | B | 116 | 47.263 | 80.485 | −8.785 | 1.00 | 24.34 |
| ATOM | 3426 | CG | PHE | B | 116 | 46.116 | 80.843 | −9.690 | 1.00 | 23.63 |
| ATOM | 3427 | CD1 | PHE | B | 116 | 46.322 | 81.640 | −10.808 | 1.00 | 23.38 |
| ATOM | 3428 | CD2 | PHE | B | 116 | 44.847 | 80.366 | −9.435 | 1.00 | 21.16 |
| ATOM | 3429 | CE1 | PHE | B | 116 | 45.270 | 81.952 | −11.653 | 1.00 | 23.56 |
| ATOM | 3430 | CE2 | PHE | B | 116 | 43.787 | 80.673 | −10.269 | 1.00 | 24.99 |
| ATOM | 3431 | CZ | PHE | B | 116 | 43.998 | 81.469 | −11.386 | 1.00 | 22.81 |
| ATOM | 3432 | C | PHE | B | 116 | 47.871 | 82.803 | −8.025 | 1.00 | 24.48 |
| ATOM | 3433 | O | PHE | B | 116 | 47.023 | 83.666 | −8.224 | 1.00 | 25.63 |
| ATOM | 3434 | N | TYR | B | 117 | 49.158 | 83.001 | −8.169 | 1.00 | 27.57 |
| ATOM | 3435 | CA | TYR | B | 117 | 49.668 | 84.268 | −8.592 | 1.00 | 30.79 |
| ATOM | 3436 | CB | TYR | B | 117 | 50.979 | 84.558 | −7.894 | 1.00 | 30.32 |
| ATOM | 3437 | CG | TYR | B | 117 | 50.828 | 84.766 | −6.409 | 1.00 | 31.52 |
| ATOM | 3438 | CD1 | TYR | B | 117 | 50.998 | 83.714 | −5.525 | 1.00 | 32.20 |
| ATOM | 3439 | CE1 | TYR | B | 117 | 50.877 | 83.897 | −4.162 | 1.00 | 33.83 |
| ATOM | 3440 | CD2 | TYR | B | 117 | 50.527 | 86.011 | −5.892 | 1.00 | 31.27 |
| ATOM | 3441 | CE2 | TYR | B | 117 | 50.401 | 86.206 | −4.526 | 1.00 | 34.81 |
| ATOM | 3442 | CZ | TYR | B | 117 | 50.579 | 85.142 | −3.666 | 1.00 | 34.02 |
| ATOM | 3443 | OH | TYR | B | 117 | 50.464 | 85.328 | −2.302 | 1.00 | 35.99 |
| ATOM | 3444 | C | TYR | B | 117 | 49.856 | 84.252 | −10.096 | 1.00 | 33.65 |
| ATOM | 3445 | O | TYR | B | 117 | 50.534 | 83.379 | −10.631 | 1.00 | 34.07 |
| ATOM | 3446 | N | SER | B | 118 | 49.242 | 85.216 | −10.764 | 1.00 | 37.02 |
| ATOM | 3447 | CA | SER | B | 118 | 49.333 | 85.336 | −12.208 | 1.00 | 39.81 |
| ATOM | 3448 | CB | SER | B | 118 | 47.956 | 85.205 | −12.857 | 1.00 | 38.58 |
| ATOM | 3449 | OG | SER | B | 118 | 47.192 | 86.387 | −12.690 | 1.00 | 38.24 |
| ATOM | 3450 | C | SER | B | 118 | 49.957 | 86.667 | −12.563 | 1.00 | 43.00 |
| ATOM | 3451 | O | SER | B | 118 | 49.586 | 87.701 | −12.005 | 1.00 | 43.48 |
| ATOM | 3452 | N | SER | B | 119 | 50.909 | 86.638 | −13.489 | 1.00 | 47.48 |
| ATOM | 3453 | CA | SER | B | 119 | 51.593 | 87.851 | −13.925 | 1.00 | 50.85 |
| ATOM | 3454 | CB | SER | B | 119 | 52.586 | 87.527 | −15.046 | 1.00 | 50.24 |
| ATOM | 3455 | OG | SER | B | 119 | 53.919 | 87.542 | −14.571 | 1.00 | 50.41 |
| ATOM | 3456 | C | SER | B | 119 | 50.582 | 88.886 | −14.412 | 1.00 | 53.19 |
| ATOM | 3457 | O | SER | B | 119 | 49.580 | 88.539 | −15.046 | 1.00 | 54.05 |
| ATOM | 3458 | N | GLY | B | 120 | 50.847 | 90.151 | −14.114 | 1.00 | 55.50 |
| ATOM | 3459 | CA | GLY | B | 120 | 49.945 | 91.200 | −14.543 | 1.00 | 59.54 |
| ATOM | 3460 | C | GLY | B | 120 | 50.379 | 91.787 | −15.869 | 1.00 | 62.44 |
| ATOM | 3461 | O | GLY | B | 120 | 50.860 | 91.067 | −16.752 | 1.00 | 63.06 |
| ATOM | 3462 | N | GLU | B | 121 | 50.239 | 93.095 | −16.006 | 1.00 | 64.20 |
| ATOM | 3463 | CA | GLU | B | 121 | 50.649 | 93.770 | −17.225 | 1.00 | 66.57 |
| ATOM | 3464 | CB | GLU | B | 121 | 49.755 | 94.988 | −17.490 | 1.00 | 67.06 |
| ATOM | 3465 | CG | GLU | B | 121 | 48.261 | 94.741 | −17.278 | 1.00 | 67.73 |
| ATOM | 3466 | CD | GLU | B | 121 | 47.817 | 93.328 | −17.636 | 1.00 | 68.42 |
| ATOM | 3467 | OE1 | GLU | B | 121 | 47.922 | 92.945 | −18.828 | 1.00 | 68.67 |
| ATOM | 3468 | OE2 | GLU | B | 121 | 47.359 | 92.604 | −16.723 | 1.00 | 67.90 |
| ATOM | 3469 | C | GLU | B | 121 | 52.115 | 94.176 | −17.098 | 1.00 | 67.64 |
| ATOM | 3470 | O | GLU | B | 121 | 52.472 | 95.334 | −17.317 | 1.00 | 68.09 |
| ATOM | 3471 | N | LYS | B | 122 | 52.952 | 93.198 | −16.705 | 1.00 | 69.00 |
| ATOM | 3472 | CA | LYS | B | 122 | 54.382 | 93.412 | −16.491 | 1.00 | 69.57 |
| ATOM | 3473 | CB | LYS | B | 122 | 55.073 | 93.868 | −17.792 | 1.00 | 70.71 |
| ATOM | 3474 | CG | LYS | B | 122 | 55.288 | 92.745 | −18.799 | 1.00 | 71.91 |
| ATOM | 3475 | CD | LYS | B | 122 | 53.980 | 92.288 | −19.436 | 1.00 | 72.78 |
| ATOM | 3476 | CE | LYS | B | 122 | 53.547 | 90.925 | −18.911 | 1.00 | 73.05 |
| ATOM | 3477 | NZ | LYS | B | 122 | 52.474 | 90.319 | −19.753 | 1.00 | 72.80 |
| ATOM | 3478 | C | LYS | B | 122 | 54.578 | 94.407 | −15.328 | 1.00 | 69.47 |
| ATOM | 3479 | O | LYS | B | 122 | 54.018 | 94.190 | −14.248 | 1.00 | 70.33 |
| ATOM | 3480 | N | LYS | B | 123 | 55.351 | 95.490 | −15.533 | 1.00 | 68.70 |
| ATOM | 3481 | CA | LYS | B | 123 | 55.584 | 96.491 | −14.479 | 1.00 | 67.63 |
| ATOM | 3482 | CB | LYS | B | 123 | 54.274 | 97.231 | −14.124 | 1.00 | 67.63 |
| ATOM | 3483 | CG | LYS | B | 123 | 53.129 | 97.002 | −15.102 | 1.00 | 67.45 |
| ATOM | 3484 | CD | LYS | B | 123 | 52.314 | 98.268 | −15.329 | 1.00 | 68.76 |
| ATOM | 3485 | CE | LYS | B | 123 | 52.831 | 99.071 | −16.521 | 1.00 | 69.10 |
| ATOM | 3486 | NZ | LYS | B | 123 | 52.538 | 98.409 | −17.828 | 1.00 | 68.94 |
| ATOM | 3487 | C | LYS | B | 123 | 56.173 | 95.834 | −13.223 | 1.00 | 66.56 |
| ATOM | 3488 | O | LYS | B | 123 | 56.777 | 94.759 | −13.300 | 1.00 | 66.55 |
| ATOM | 3489 | N | ASP | B | 124 | 55.979 | 96.469 | −12.065 | 1.00 | 65.01 |
| ATOM | 3490 | CA | ASP | B | 124 | 56.471 | 95.911 | −10.806 | 1.00 | 63.86 |
| ATOM | 3491 | CB | ASP | B | 124 | 57.425 | 96.863 | −10.081 | 1.00 | 64.99 |
| ATOM | 3492 | CG | ASP | B | 124 | 58.593 | 96.115 | −9.460 | 1.00 | 66.12 |
| ATOM | 3493 | OD1 | ASP | B | 124 | 58.352 | 95.082 | −8.791 | 1.00 | 65.87 |
| ATOM | 3494 | OD2 | ASP | B | 124 | 59.749 | 96.554 | −9.653 | 1.00 | 67.14 |
| ATOM | 3495 | C | ASP | B | 124 | 55.301 | 95.508 | −9.911 | 1.00 | 62.01 |
| ATOM | 3496 | O | ASP | B | 124 | 55.232 | 95.869 | −8.729 | 1.00 | 61.22 |
| ATOM | 3497 | N | GLU | B | 125 | 54.374 | 94.764 | −10.511 | 1.00 | 59.15 |
| ATOM | 3498 | CA | GLU | B | 125 | 53.175 | 94.297 | −9.842 | 1.00 | 55.30 |
| ATOM | 3499 | CB | GLU | B | 125 | 52.028 | 95.276 | −10.122 | 1.00 | 56.22 |
| ATOM | 3500 | CG | GLU | B | 125 | 52.053 | 95.871 | −11.528 | 1.00 | 56.42 |
| ATOM | 3501 | CD | GLU | B | 125 | 50.810 | 96.673 | −11.870 | 1.00 | 57.43 |
| ATOM | 3502 | OE1 | GLU | B | 125 | 50.176 | 96.375 | −12.911 | 1.00 | 56.96 |
| ATOM | 3503 | OE2 | GLU | B | 125 | 50.474 | 97.601 | −11.103 | 1.00 | 56.77 |
| ATOM | 3504 | C | GLU | B | 125 | 52.785 | 92.906 | −10.337 | 1.00 | 53.36 |
| ATOM | 3505 | O | GLU | B | 125 | 53.303 | 92.413 | −11.350 | 1.00 | 52.90 |
| ATOM | 3506 | N | VAL | B | 126 | 51.855 | 92.288 | −9.623 | 1.00 | 50.42 |
| ATOM | 3507 | CA | VAL | B | 126 | 51.358 | 90.962 | −9.969 | 1.00 | 46.95 |
| ATOM | 3508 | CB | VAL | B | 126 | 52.242 | 89.843 | −9.371 | 1.00 | 47.91 |
| ATOM | 3509 | CG1 | VAL | B | 126 | 52.128 | 89.797 | −7.851 | 1.00 | 48.65 |
| ATOM | 3510 | CG2 | VAL | B | 126 | 51.889 | 88.500 | −9.989 | 1.00 | 47.79 |
| ATOM | 3511 | C | VAL | B | 126 | 49.911 | 90.802 | −9.514 | 1.00 | 43.29 |
| ATOM | 3512 | O | VAL | B | 126 | 49.384 | 91.659 | −8.814 | 1.00 | 43.62 |
| ATOM | 3513 | N | TYR | B | 127 | 49.271 | 89.716 | −9.921 | 1.00 | 40.00 |
| ATOM | 3514 | CA | TYR | B | 127 | 47.887 | 89.479 | −9.545 | 1.00 | 37.12 |
| ATOM | 3515 | CB | TYR | B | 127 | 46.986 | 89.395 | −10.783 | 1.00 | 39.32 |
| ATOM | 3516 | CG | TYR | B | 127 | 46.672 | 90.728 | −11.434 | 1.00 | 42.49 |
| ATOM | 3517 | CD1 | TYR | B | 127 | 47.438 | 91.197 | −12.501 | 1.00 | 42.64 |
| ATOM | 3518 | CE1 | TYR | B | 127 | 47.160 | 92.407 | −13.109 | 1.00 | 42.93 |
| ATOM | 3519 | CD2 | TYR | B | 127 | 45.612 | 91.513 | −10.994 | 1.00 | 41.97 |
| ATOM | 3520 | CE2 | TYR | B | 127 | 45.327 | 92.728 | −11.597 | 1.00 | 43.42 |
| ATOM | 3521 | CZ | TYR | B | 127 | 46.105 | 93.170 | −12.656 | 1.00 | 43.75 |
| ATOM | 3522 | OH | TYR | B | 127 | 45.822 | 94.377 | −13.264 | 1.00 | 44.38 |
| ATOM | 3523 | C | TYR | B | 127 | 47.727 | 88.203 | −8.722 | 1.00 | 35.28 |
| ATOM | 3524 | O | TYR | B | 127 | 48.171 | 87.126 | −9.133 | 1.00 | 34.31 |
| ATOM | 3525 | N | LEU | B | 128 | 47.045 | 88.327 | −7.584 | 1.00 | 30.55 |
| ATOM | 3526 | CA | LEU | B | 128 | 46.768 | 87.181 | −6.727 | 1.00 | 26.58 |
| ATOM | 3527 | CB | LEU | B | 128 | 46.858 | 87.554 | −5.236 | 1.00 | 24.20 |
| ATOM | 3528 | CG | LEU | B | 128 | 46.128 | 86.602 | −4.280 | 1.00 | 22.15 |
| ATOM | 3529 | CD1 | LEU | B | 128 | 46.759 | 85.218 | −4.328 | 1.00 | 17.31 |
| ATOM | 3530 | CD2 | LEU | B | 128 | 46.107 | 87.156 | −2.854 | 1.00 | 22.87 |
| ATOM | 3531 | C | LEU | B | 128 | 45.376 | 86.677 | −7.040 | 1.00 | 22.48 |
| ATOM | 3532 | O | LEU | B | 128 | 44.424 | 87.447 | −7.019 | 1.00 | 24.60 |
| ATOM | 3533 | N | ASN | B | 129 | 45.252 | 85.400 | −7.333 | 1.00 | 21.14 |
| ATOM | 3534 | CA | ASN | B | 129 | 43.949 | 84.834 | −7.650 | 1.00 | 20.80 |
| ATOM | 3535 | CB | ASN | B | 129 | 43.957 | 84.125 | −9.013 | 1.00 | 21.81 |
| ATOM | 3536 | CG | ASN | B | 129 | 44.312 | 85.073 | −10.152 | 1.00 | 21.32 |
| ATOM | 3537 | OD1 | ASN | B | 129 | 45.468 | 85.447 | −10.318 | 1.00 | 22.73 |
| ATOM | 3538 | ND2 | ASN | B | 129 | 43.312 | 85.479 | −10.919 | 1.00 | 21.75 |
| ATOM | 3539 | C | ASN | B | 129 | 43.472 | 83.893 | −6.556 | 1.00 | 20.70 |
| ATOM | 3540 | O | ASN | B | 129 | 44.151 | 82.936 | −6.207 | 1.00 | 21.20 |
| ATOM | 3541 | N | LEU | B | 130 | 42.302 | 84.183 | −6.026 | 1.00 | 20.89 |
| ATOM | 3542 | CA | LEU | B | 130 | 41.717 | 83.384 | −4.974 | 1.00 | 21.65 |
| ATOM | 3543 | CB | LEU | B | 130 | 41.290 | 84.270 | −3.787 | 1.00 | 22.41 |
| ATOM | 3544 | CG | LEU | B | 130 | 42.398 | 85.111 | −3.150 | 1.00 | 22.43 |
| ATOM | 3545 | CD1 | LEU | B | 130 | 41.814 | 86.206 | −2.267 | 1.00 | 23.86 |
| ATOM | 3546 | CD2 | LEU | B | 130 | 43.361 | 84.236 | −2.370 | 1.00 | 22.19 |
| ATOM | 3547 | C | LEU | B | 130 | 40.533 | 82.634 | −5.511 | 1.00 | 19.68 |
| ATOM | 3548 | O | LEU | B | 130 | 39.582 | 83.240 | −5.980 | 1.00 | 21.26 |
| ATOM | 3549 | N | VAL | B | 131 | 40.600 | 81.314 | −5.442 | 1.00 | 18.58 |
| ATOM | 3550 | CA | VAL | B | 131 | 39.521 | 80.467 | −5.926 | 1.00 | 20.51 |
| ATOM | 3551 | CB | VAL | B | 131 | 40.056 | 79.204 | −6.629 | 1.00 | 16.75 |
| ATOM | 3552 | CG1 | VAL | B | 131 | 38.945 | 78.497 | −7.366 | 1.00 | 14.65 |
| ATOM | 3553 | CG2 | VAL | B | 131 | 41.199 | 79.561 | −7.558 | 1.00 | 20.04 |
| ATOM | 3554 | C | VAL | B | 131 | 38.620 | 80.082 | −4.768 | 1.00 | 20.58 |
| ATOM | 3555 | O | VAL | B | 131 | 38.929 | 79.179 | −3.997 | 1.00 | 21.73 |
| ATOM | 3556 | N | LEU | B | 132 | 37.536 | 80.827 | −4.644 | 1.00 | 24.39 |
| ATOM | 3557 | CA | LEU | B | 132 | 36.567 | 80.675 | −3.572 | 1.00 | 25.11 |
| ATOM | 3558 | CB | LEU | B | 132 | 36.152 | 82.066 | −3.090 | 1.00 | 21.67 |
| ATOM | 3559 | CC | LEU | B | 132 | 37.304 | 83.031 | −2.830 | 1.00 | 24.26 |
| ATOM | 3560 | CD1 | LEU | B | 132 | 36.902 | 84.472 | −3.122 | 1.00 | 21.82 |
| ATOM | 3561 | CD2 | LEU | B | 132 | 37.809 | 82.870 | −1.403 | 1.00 | 22.44 |
| ATOM | 3562 | C | LEU | B | 132 | 35.329 | 79.922 | −3.985 | 1.00 | 27.80 |
| ATOM | 3563 | O | LEU | B | 132 | 34.982 | 79.844 | −5.172 | 1.00 | 29.52 |
| ATOM | 3564 | N | ASP | B | 133 | 34.641 | 79.419 | −2.972 | 1.00 | 27.43 |
| ATOM | 3565 | CA | ASP | B | 133 | 33.388 | 78.709 | −3.126 | 1.00 | 28.52 |
| ATOM | 3566 | CB | ASP | B | 133 | 32.825 | 78.410 | −1.728 | 1.00 | 30.97 |
| ATOM | 3567 | CG | ASP | B | 133 | 33.214 | 77.071 | −1.149 | 1.00 | 33.42 |
| ATOM | 3568 | OD1 | ASP | B | 133 | 33.227 | 76.961 | 0.097 | 1.00 | 33.58 |
| ATOM | 3569 | OD2 | ASP | B | 133 | 33.478 | 76.128 | −1.931 | 1.00 | 35.77 |
| ATOM | 3570 | C | ASP | B | 133 | 32.391 | 79.647 | −3.766 | 1.00 | 28.01 |
| ATOM | 3571 | O | ASP | B | 133 | 32.288 | 80.810 | −3.362 | 1.00 | 27.90 |
| ATOM | 3572 | N | TYR | B | 134 | 31.632 | 79.151 | −4.724 | 1.00 | 27.47 |
| ATOM | 3573 | CA | TYR | B | 134 | 30.627 | 79.985 | −5.354 | 1.00 | 26.54 |
| ATOM | 3574 | CB | TYR | B | 134 | 30.549 | 79.772 | −6.873 | 1.00 | 27.48 |
| ATOM | 3575 | CG | TYR | B | 134 | 29.531 | 80.682 | −7.523 | 1.00 | 25.44 |
| ATOM | 3576 | CD1 | TYR | B | 134 | 29.861 | 81.981 | −7.866 | 1.00 | 25.68 |
| ATOM | 3577 | CE1 | TYR | B | 134 | 28.932 | 82.831 | −8.423 | 1.00 | 28.12 |
| ATOM | 3578 | CD2 | TYR | B | 134 | 28.232 | 80.251 | −7.756 | 1.00 | 27.07 |
| ATOM | 3579 | CE2 | TYR | B | 134 | 27.296 | 81.095 | −8.320 | 1.00 | 28.14 |
| ATOM | 3580 | CZ | TYR | B | 134 | 27.654 | 82.384 | −8.649 | 1.00 | 27.66 |
| ATOM | 3581 | OH | TYR | B | 134 | 26.725 | 83.235 | −9.198 | 1.00 | 32.43 |
| ATOM | 3582 | C | TYR | B | 134 | 29.261 | 79.773 | −4.722 | 1.00 | 26.49 |
| ATOM | 3583 | O | TYR | B | 134 | 28.804 | 78.645 | −4.546 | 1.00 | 29.00 |
| ATOM | 3584 | N | VAL | B | 135 | 28.610 | 80.873 | −4.410 | 1.00 | 26.03 |
| ATOM | 3585 | CA | VAL | B | 135 | 27.281 | 80.857 | −3.832 | 1.00 | 27.01 |
| ATOM | 3586 | CB | VAL | B | 135 | 27.302 | 81.038 | −2.296 | 1.00 | 27.39 |
| ATOM | 3587 | CG1 | VAL | B | 135 | 25.928 | 80.771 | −1.710 | 1.00 | 25.29 |
| ATOM | 3588 | CG2 | VAL | B | 135 | 28.322 | 80.090 | −1.680 | 1.00 | 26.48 |
| ATOM | 3589 | C | VAL | B | 135 | 26.464 | 81.936 | −4.532 | 1.00 | 28.41 |
| ATOM | 3590 | O | VAL | B | 135 | 26.934 | 83.059 | −4.695 | 1.00 | 30.10 |
| ATOM | 3591 | N | PRO | B | 136 | 25.267 | 81.581 | −5.028 | 1.00 | 29.44 |
| ATOM | 3592 | CD | PRO | B | 136 | 24.680 | 80.227 | −4.933 | 1.00 | 29.98 |
| ATOM | 3593 | CA | PRO | B | 136 | 24.411 | 82.496 | −5.806 | 1.00 | 29.90 |
| ATOM | 3594 | CB | PRO | B | 136 | 23.382 | 81.546 | −6.434 | 1.00 | 30.14 |
| ATOM | 3595 | CG | PRO | B | 136 | 23.287 | 80.419 | −5.460 | 1.00 | 30.40 |
| ATOM | 3596 | C | PRO | B | 136 | 23.696 | 83.605 | −5.021 | 1.00 | 29.47 |
| ATOM | 3597 | O | PRO | B | 136 | 23.857 | 84.783 | −5.324 | 1.00 | 29.46 |
| ATOM | 3598 | N | GLU | B | 137 | 22.860 | 83.229 | −4.068 | 1.00 | 28.02 |
| ATOM | 3599 | CA | GLU | B | 137 | 22.082 | 84.209 | −3.315 | 1.00 | 29.48 |
| ATOM | 3600 | CB | GLU | B | 137 | 20.783 | 83.560 | −2.816 | 1.00 | 31.02 |
| ATOM | 3601 | CG | GLU | B | 137 | 19.533 | 83.933 | −3.601 | 1.00 | 34.32 |
| ATOM | 3602 | CD | GLU | B | 137 | 19.526 | 85.381 | −4.047 | 1.00 | 37.83 |
| ATOM | 3603 | OE1 | GLU | B | 137 | 19.693 | 85.627 | −5.260 | 1.00 | 39.93 |
| ATOM | 3604 | OE2 | GLU | B | 137 | 19.358 | 86.269 | −3.185 | 1.00 | 39.76 |
| ATOM | 3605 | C | GLU | B | 137 | 22.846 | 84.828 | −2.138 | 1.00 | 27.83 |
| ATOM | 3606 | O | GLU | B | 137 | 23.893 | 84.340 | −1.733 | 1.00 | 28.10 |
| ATOM | 3607 | N | THR | B | 138 | 22.266 | 85.890 | −1.581 | 1.00 | 27.83 |
| ATOM | 3608 | CA | THR | B | 138 | 22.806 | 86.602 | −0.406 | 1.00 | 25.21 |
| ATOM | 3609 | CB | THR | B | 138 | 23.650 | 87.814 | −0.781 | 1.00 | 22.17 |
| ATOM | 3610 | OG1 | THR | B | 138 | 22.851 | 88.827 | −1.358 | 1.00 | 22.19 |
| ATOM | 3611 | CG2 | THR | B | 138 | 24.827 | 87.524 | −1.675 | 1.00 | 24.71 |
| ATOM | 3612 | C | THR | B | 138 | 21.648 | 87.088 | 0.455 | 1.00 | 23.46 |
| ATOM | 3613 | O | THR | B | 138 | 20.558 | 87.353 | −0.061 | 1.00 | 22.29 |
| ATOM | 3614 | N | VAL | B | 139 | 21.887 | 87.220 | 1.767 | 1.00 | 23.34 |
| ATOM | 3615 | CA | VAL | B | 139 | 20.840 | 87.689 | 2.678 | 1.00 | 18.25 |
| ATOM | 3616 | CB | VAL | B | 139 | 21.342 | 87.819 | 4.138 | 1.00 | 19.10 |
| ATOM | 3617 | CG1 | VAL | B | 139 | 20.241 | 88.386 | 5.030 | 1.00 | 17.18 |
| ATOM | 3618 | CG2 | VAL | B | 139 | 21.787 | 86.469 | 4.651 | 1.00 | 16.04 |
| ATOM | 3619 | C | VAL | B | 139 | 20.263 | 89.013 | 2.201 | 1.00 | 16.00 |
| ATOM | 3620 | O | VAL | B | 139 | 19.056 | 89.222 | 2.268 | 1.00 | 19.23 |
| ATOM | 3621 | N | TYR | B | 140 | 21.131 | 89.892 | 1.696 | 1.00 | 17.31 |
| ATOM | 3622 | CA | TYR | B | 140 | 20.701 | 91.194 | 1.182 | 1.00 | 18.53 |
| ATOM | 3623 | CB | TYR | B | 140 | 21.873 | 91.978 | 0.586 | 1.00 | 17.88 |
| ATOM | 3624 | CG | TYR | B | 140 | 21.438 | 93.261 | −0.094 | 1.00 | 22.14 |
| ATOM | 3625 | CD1 | TYR | B | 140 | 21.414 | 94.456 | 0.599 | 1.00 | 22.08 |
| ATOM | 3626 | CE1 | TYR | B | 140 | 20.985 | 95.627 | 0.010 | 1.00 | 24.00 |
| ATOM | 3627 | CD2 | TYR | B | 140 | 21.013 | 93.270 | −1.424 | 1.00 | 25.34 |
| ATOM | 3628 | CE2 | TYR | B | 140 | 20.585 | 94.439 | −2.031 | 1.00 | 25.37 |
| ATOM | 3629 | CZ | TYR | B | 140 | 20.572 | 95.618 | −1.306 | 1.00 | 28.17 |
| ATOM | 3630 | OH | TYR | B | 140 | 20.144 | 96.794 | −1.898 | 1.00 | 29.68 |
| ATOM | 3631 | C | TYR | B | 140 | 19.593 | 91.054 | 0.127 | 1.00 | 22.25 |
| ATOM | 3632 | O | TYR | B | 140 | 18.589 | 91.764 | 0.177 | 1.00 | 23.33 |
| ATOM | 3633 | N | ARG | B | 141 | 19.781 | 90.155 | −0.844 | 1.00 | 25.14 |
| ATOM | 3634 | CA | ARG | B | 141 | 18.782 | 89.976 | −1.897 | 1.00 | 25.99 |
| ATOM | 3635 | CB | ARG | B | 141 | 19.354 | 89.162 | −3.062 | 1.00 | 29.20 |
| ATOM | 3636 | CG | ARG | B | 141 | 20.159 | 90.003 | −4.045 | 1.00 | 34.68 |
| ATOM | 3637 | CD | ARG | B | 141 | 21.396 | 89.263 | −4.550 | 1.00 | 39.35 |
| ATOM | 3638 | NE | ARG | B | 141 | 21.049 | 88.183 | −5.473 | 1.00 | 45.84 |
| ATOM | 3639 | CZ | ARG | B | 141 | 20.518 | 88.374 | −6.691 | 1.00 | 48.75 |
| ATOM | 3640 | NH1 | ARG | B | 141 | 20.306 | 89.608 | −7.159 | 1.00 | 50.02 |
| ATOM | 3641 | NH2 | ARG | B | 141 | 20.204 | 87.324 | −7.448 | 1.00 | 49.98 |
| ATOM | 3642 | C | ARG | B | 141 | 17.532 | 89.321 | −1.341 | 1.00 | 25.44 |
| ATOM | 3643 | O | ARG | B | 141 | 16.410 | 89.751 | −1.626 | 1.00 | 25.68 |
| ATOM | 3644 | N | VAL | B | 142 | 17.716 | 88.287 | −0.532 | 1.00 | 25.77 |
| ATOM | 3645 | CA | VAL | B | 142 | 16.573 | 87.613 | 0.057 | 1.00 | 25.48 |
| ATOM | 3646 | CB | VAL | B | 142 | 16.994 | 86.424 | 0.945 | 1.00 | 26.10 |
| ATOM | 3647 | CG1 | VAL | B | 142 | 15.814 | 85.905 | 1.747 | 1.00 | 25.96 |
| ATOM | 3648 | CG2 | VAL | B | 142 | 17.586 | 85.313 | 0.088 | 1.00 | 27.08 |
| ATOM | 3649 | C | VAL | B | 142 | 15.734 | 88.610 | 0.850 | 1.00 | 25.06 |
| ATOM | 3650 | O | VAL | B | 142 | 14.512 | 88.600 | 0.763 | 1.00 | 27.10 |
| ATOM | 3651 | N | ALA | B | 143 | 16.409 | 89.489 | 1.598 | 1.00 | 25.37 |
| ATOM | 3652 | CA | ALA | B | 143 | 15.738 | 90.521 | 2.397 | 1.00 | 25.38 |
| ATOM | 3653 | CB | ALA | B | 143 | 16.744 | 91.238 | 3.283 | 1.00 | 23.30 |
| ATOM | 3654 | C | ALA | B | 143 | 15.005 | 91.522 | 1.507 | 1.00 | 25.25 |
| ATOM | 3655 | O | ALA | B | 143 | 13.821 | 91.794 | 1.712 | 1.00 | 25.01 |
| ATOM | 3656 | N | ARG | B | 144 | 15.714 | 92.060 | 0.508 | 1.00 | 27.14 |
| ATOM | 3657 | CA | ARG | B | 144 | 15.125 | 93.023 | −0.427 | 1.00 | 27.56 |
| ATOM | 3658 | CB | ARG | B | 144 | 16.141 | 93.423 | −1.499 | 1.00 | 28.99 |
| ATOM | 3659 | CG | ARG | B | 144 | 15.608 | 94.477 | −2.466 | 1.00 | 31.54 |
| ATOM | 3660 | CD | ARG | B | 144 | 16.704 | 95.035 | −3.353 | 1.00 | 32.11 |
| ATOM | 3661 | NE | ARG | B | 144 | 17.293 | 94.000 | −4.198 | 1.00 | 35.33 |
| ATOM | 3662 | CZ | ARG | B | 144 | 18.226 | 94.223 | −5.123 | 1.00 | 35.49 |
| ATOM | 3663 | NH1 | ARG | B | 144 | 18.678 | 95.457 | −5.357 | 1.00 | 35.51 |
| ATOM | 3664 | NH2 | ARG | B | 144 | 18.709 | 93.199 | −5.818 | 1.00 | 36.90 |
| ATOM | 3665 | C | ARG | B | 144 | 13.872 | 92.448 | −1.089 | 1.00 | 27.74 |
| ATOM | 3666 | O | ARG | B | 144 | 12.865 | 93.147 | −1.255 | 1.00 | 30.06 |
| ATOM | 3667 | N | HIS | B | 145 | 13.930 | 91.171 | −1.440 | 1.00 | 27.38 |
| ATOM | 3668 | CA | HIS | B | 145 | 12.800 | 90.494 | −2.057 | 1.00 | 29.82 |
| ATOM | 3669 | CB | HIS | B | 145 | 13.153 | 89.033 | −2.358 | 1.00 | 33.31 |
| ATOM | 3670 | CG | HIS | B | 145 | 12.098 | 88.302 | −3.123 | 1.00 | 38.70 |
| ATOM | 3671 | CD2 | HIS | B | 145 | 11.584 | 87.058 | −2.960 | 1.00 | 41.14 |
| ATOM | 3672 | ND1 | HIS | B | 145 | 11.429 | 88.852 | −4.202 | 1.00 | 41.54 |
| ATOM | 3673 | CE1 | HIS | B | 145 | 10.549 | 87.978 | −4.665 | 1.00 | 42.00 |
| ATOM | 3674 | NE2 | HIS | B | 145 | 10.623 | 86.880 | −3.929 | 1.00 | 41.98 |
| ATOM | 3675 | C | HIS | B | 145 | 11.558 | 90.575 | −1.167 | 1.00 | 29.00 |
| ATOM | 3676 | O | HIS | B | 145 | 10.507 | 91.053 | −1.598 | 1.00 | 30.08 |
| ATOM | 3677 | N | TYR | B | 146 | 11.683 | 90.120 | 0.081 | 1.00 | 28.13 |
| ATOM | 3678 | CA | TYR | B | 146 | 10.565 | 90.167 | 1.016 | 1.00 | 25.81 |
| ATOM | 3679 | CB | TYR | B | 146 | 10.919 | 89.471 | 2.332 | 1.00 | 23.53 |
| ATOM | 3680 | CG | TYR | B | 146 | 10.782 | 87.978 | 2.242 | 1.00 | 19.02 |
| ATOM | 3681 | CD1 | TYR | B | 146 | 11.846 | 87.193 | 1.826 | 1.00 | 18.11 |
| ATOM | 3682 | CE1 | TYR | B | 146 | 11.719 | 85.829 | 1.702 | 1.00 | 17.01 |
| ATOM | 3683 | CD2 | TYR | B | 146 | 9.581 | 87.357 | 2.536 | 1.00 | 19.30 |
| ATOM | 3684 | CE2 | TYR | B | 146 | 9.446 | 85.991 | 2.421 | 1.00 | 18.95 |
| ATOM | 3685 | CZ | TYR | B | 146 | 10.520 | 85.233 | 2.001 | 1.00 | 18.86 |
| ATOM | 3686 | OH | TYR | B | 146 | 10.388 | 83.873 | 1.879 | 1.00 | 23.47 |
| ATOM | 3687 | C | TYR | B | 146 | 10.133 | 91.603 | 1.271 | 1.00 | 26.52 |
| ATOM | 3688 | O | TYR | B | 146 | 8.943 | 91.910 | 1.271 | 1.00 | 27.02 |
| ATOM | 3689 | N | SER | B | 147 | 11.114 | 92.476 | 1.480 | 1.00 | 29.00 |
| ATOM | 3690 | CA | SER | B | 147 | 10.864 | 93.890 | 1.737 | 1.00 | 31.27 |
| ATOM | 3691 | CB | SER | B | 147 | 12.189 | 94.631 | 1.913 | 1.00 | 32.96 |
| ATOM | 3692 | OG | SER | B | 147 | 12.065 | 95.669 | 2.870 | 1.00 | 37.24 |
| ATOM | 3693 | C | SER | B | 147 | 10.040 | 94.540 | 0.622 | 1.00 | 33.59 |
| ATOM | 3694 | O | SER | B | 147 | 9.102 | 95.290 | 0.894 | 1.00 | 34.13 |
| ATOM | 3695 | N | ARG | B | 148 | 10.392 | 94.254 | −0.635 | 1.00 | 34.97 |
| ATOM | 3696 | CA | ARG | B | 148 | 9.671 | 94.825 | −1.774 | 1.00 | 35.55 |
| ATOM | 3697 | CB | ARG | B | 148 | 10.387 | 94.494 | −3.085 | 1.00 | 36.04 |
| ATOM | 3698 | CG | ARG | B | 148 | 11.721 | 95.209 | −3.245 | 1.00 | 36.48 |
| ATOM | 3699 | CD | ARG | B | 148 | 12.555 | 94.593 | −4.360 | 1.00 | 37.75 |
| ATOM | 3700 | NE | ARG | B | 148 | 13.591 | 95.508 | −4.847 | 1.00 | 40.00 |
| ATOM | 3701 | CZ | ARG | B | 148 | 14.421 | 95.236 | −5.860 | 1.00 | 40.69 |
| ATOM | 3702 | NH1 | ARG | B | 148 | 14.359 | 94.070 | −6.494 | 1.00 | 40.58 |
| ATOM | 3703 | NH2 | ARG | B | 148 | 15.321 | 96.137 | −6.237 | 1.00 | 41.76 |
| ATOM | 3704 | C | ARG | B | 148 | 8.228 | 94.325 | −1.816 | 1.00 | 35.37 |
| ATOM | 3705 | O | ARG | B | 148 | 7.309 | 95.082 | −2.131 | 1.00 | 36.98 |
| ATOM | 3706 | N | ALA | B | 149 | 8.032 | 93.053 | −1.484 | 1.00 | 34.57 |
| ATOM | 3707 | CA | ALA | B | 149 | 6.697 | 92.460 | −1.476 | 1.00 | 33.48 |
| ATOM | 3708 | CB | ALA | B | 149 | 6.780 | 90.956 | −1.675 | 1.00 | 33.34 |
| ATOM | 3709 | C | ALA | B | 149 | 5.918 | 92.790 | −0.203 | 1.00 | 33.34 |
| ATOM | 3710 | O | ALA | B | 149 | 4.840 | 92.253 | 0.004 | 1.00 | 32.82 |
| ATOM | 3711 | N | LYS | B | 150 | 6.469 | 93.676 | 0.644 | 1.00 | 35.63 |
| ATOM | 3712 | CA | LYS | B | 150 | 5.824 | 94.076 | 1.899 | 1.00 | 35.93 |
| ATOM | 3713 | CB | LYS | B | 150 | 4.575 | 94.916 | 1.624 | 1.00 | 36.80 |
| ATOM | 3714 | CG | LYS | B | 150 | 4.887 | 96.330 | 1.157 | 1.00 | 37.59 |
| ATOM | 3715 | CD | LYS | B | 150 | 5.543 | 97.162 | 2.254 | 1.00 | 38.68 |
| ATOM | 3716 | CE | LYS | B | 150 | 6.378 | 98.302 | 1.674 | 1.00 | 39.17 |
| ATOM | 3717 | NZ | LYS | B | 150 | 5.826 | 99.646 | 2.024 | 1.00 | 40.69 |
| ATOM | 3718 | C | LYS | B | 150 | 5.486 | 92.851 | 2.745 | 1.00 | 35.84 |
| ATOM | 3719 | O | LYS | B | 150 | 4.357 | 92.658 | 3.192 | 1.00 | 35.50 |
| ATOM | 3720 | N | GLN | B | 151 | 6.495 | 92.026 | 2.938 | 1.00 | 36.24 |
| ATOM | 3721 | CA | GLN | B | 151 | 6.387 | 90.802 | 3.701 | 1.00 | 35.89 |
| ATOM | 3722 | CB | GLN | B | 151 | 6.400 | 89.605 | 2.749 | 1.00 | 37.28 |
| ATOM | 3723 | CG | GLN | B | 151 | 5.187 | 88.703 | 2.839 | 1.00 | 38.89 |
| ATOM | 3724 | CD | GLN | B | 151 | 4.668 | 88.316 | 1.467 | 1.00 | 41.33 |
| ATOM | 3725 | OE1 | GLN | B | 151 | 5.446 | 88.066 | 0.541 | 1.00 | 41.35 |
| ATOM | 3726 | NE2 | GLN | B | 151 | 3.347 | 88.268 | 1.330 | 1.00 | 42.23 |
| ATOM | 3727 | C | GLN | B | 151 | 7.583 | 90.687 | 4.612 | 1.00 | 35.52 |
| ATOM | 3728 | O | GLN | B | 151 | 8.669 | 91.166 | 4.285 | 1.00 | 35.18 |
| ATOM | 3729 | N | THR | B | 152 | 7.394 | 90.033 | 5.741 | 1.00 | 35.74 |
| ATOM | 3730 | CA | THR | B | 152 | 8.482 | 89.843 | 6.677 | 1.00 | 35.93 |
| ATOM | 3731 | CB | THR | B | 152 | 7.980 | 89.947 | 8.131 | 1.00 | 36.72 |
| ATOM | 3732 | OG1 | THR | B | 152 | 8.812 | 89.221 | 9.025 | 1.00 | 40.21 |
| ATOM | 3733 | CG2 | THR | B | 152 | 6.551 | 89.487 | 8.337 | 1.00 | 36.78 |
| ATOM | 3734 | C | THR | B | 152 | 9.145 | 88.500 | 6.416 | 1.00 | 32.95 |
| ATOM | 3735 | O | THR | B | 152 | 8.466 | 87.501 | 6.200 | 1.00 | 33.95 |
| ATOM | 3736 | N | LEU | B | 153 | 10.464 | 88.474 | 6.438 | 1.00 | 31.92 |
| ATOM | 3737 | CA | LEU | B | 153 | 11.184 | 87.232 | 6.217 | 1.00 | 30.87 |
| ATOM | 3738 | CB | LEU | B | 153 | 12.686 | 87.508 | 6.165 | 1.00 | 31.70 |
| ATOM | 3739 | CG | LEU | B | 153 | 13.605 | 86.301 | 5.982 | 1.00 | 32.12 |
| ATOM | 3740 | CD1 | LEU | B | 153 | 13.379 | 85.648 | 4.627 | 1.00 | 32.63 |
| ATOM | 3741 | CD2 | LEU | B | 153 | 15.055 | 86.721 | 6.139 | 1.00 | 33.53 |
| ATOM | 3742 | C | LEU | B | 153 | 10.860 | 86.277 | 7.356 | 1.00 | 30.19 |
| ATOM | 3743 | O | LEU | B | 153 | 11.119 | 86.594 | 8.510 | 1.00 | 31.69 |
| ATOM | 3744 | N | PRO | B | 154 | 10.266 | 85.102 | 7.055 | 1.00 | 29.41 |
| ATOM | 3745 | CD | PRO | B | 154 | 9.898 | 84.640 | 5.696 | 1.00 | 27.70 |
| ATOM | 3746 | CA | PRO | B | 154 | 9.891 | 84.124 | 8.080 | 1.00 | 27.35 |
| ATOM | 3747 | CB | PRO | B | 154 | 9.714 | 82.836 | 7.280 | 1.00 | 28.38 |
| ATOM | 3748 | CG | PRO | B | 154 | 9.215 | 83.315 | 5.949 | 1.00 | 28.03 |
| ATOM | 3749 | C | PRO | B | 154 | 10.962 | 83.978 | 9.159 | 1.00 | 25.58 |
| ATOM | 3750 | O | PRO | B | 154 | 12.153 | 83.809 | 8.863 | 1.00 | 25.12 |
| ATOM | 3751 | N | VAL | B | 155 | 10.527 | 84.082 | 10.418 | 1.00 | 24.34 |
| ATOM | 3752 | CA | VAL | B | 155 | 11.435 | 83.997 | 11.567 | 1.00 | 22.14 |
| ATOM | 3753 | CB | VAL | B | 155 | 10.689 | 84.144 | 12.923 | 1.00 | 23.51 |
| ATOM | 3754 | CG1 | VAL | B | 155 | 11.560 | 83.672 | 14.082 | 1.00 | 22.33 |
| ATOM | 3755 | CG2 | VAL | B | 155 | 10.293 | 85.598 | 13.127 | 1.00 | 20.79 |
| ATOM | 3756 | C | VAL | B | 155 | 12.346 | 82.768 | 11.539 | 1.00 | 19.24 |
| ATOM | 3757 | O | VAL | B | 155 | 13.489 | 82.840 | 12.005 | 1.00 | 21.30 |
| ATOM | 3758 | N | ILE | B | 156 | 11.872 | 81.644 | 10.981 | 1.00 | 19.80 |
| ATOM | 3759 | CA | ILE | B | 156 | 12.718 | 80.441 | 10.908 | 1.00 | 20.19 |
| ATOM | 3760 | CB | ILE | B | 156 | 11.980 | 79.211 | 10.311 | 1.00 | 23.70 |
| ATOM | 3761 | CG2 | ILE | B | 156 | 11.512 | 79.480 | 8.882 | 1.00 | 20.74 |
| ATOM | 3762 | CG1 | ILE | B | 156 | 12.872 | 77.963 | 10.344 | 1.00 | 23.22 |
| ATOM | 3763 | CD1 | ILE | B | 156 | 13.199 | 77.470 | 11.740 | 1.00 | 27.05 |
| ATOM | 3764 | C | ILE | B | 156 | 14.012 | 80.713 | 10.125 | 1.00 | 20.85 |
| ATOM | 3765 | O | ILE | B | 156 | 15.064 | 80.187 | 10.460 | 1.00 | 23.05 |
| ATOM | 3766 | N | TYR | B | 157 | 13.931 | 81.546 | 9.088 | 1.00 | 21.98 |
| ATOM | 3767 | CA | TYR | B | 157 | 15.103 | 81.881 | 8.267 | 1.00 | 21.22 |
| ATOM | 3768 | CB | TYR | B | 157 | 14.660 | 82.525 | 6.944 | 1.00 | 23.30 |
| ATOM | 3769 | CG | TYR | B | 157 | 13.948 | 81.576 | 6.027 | 1.00 | 23.20 |
| ATOM | 3770 | CD1 | TYR | B | 157 | 12.631 | 81.796 | 5.662 | 1.00 | 26.34 |
| ATOM | 3771 | CE1 | TYR | B | 157 | 11.970 | 80.918 | 4.830 | 1.00 | 26.40 |
| ATOM | 3772 | CD2 | TYR | B | 157 | 14.592 | 80.450 | 5.531 | 1.00 | 24.66 |
| ATOM | 3773 | CE2 | TYR | B | 157 | 13.941 | 79.568 | 4.694 | 1.00 | 25.59 |
| ATOM | 3774 | CZ | TYR | B | 157 | 12.634 | 79.806 | 4.350 | 1.00 | 27.07 |
| ATOM | 3775 | OH | TYR | B | 157 | 11.976 | 78.922 | 3.532 | 1.00 | 32.84 |
| ATOM | 3776 | C | TYR | B | 157 | 16.057 | 82.819 | 8.990 | 1.00 | 20.56 |
| ATOM | 3777 | O | TYR | B | 157 | 17.281 | 82.735 | 8.817 | 1.00 | 19.78 |
| ATOM | 3778 | N | VAL | B | 158 | 15.496 | 83.717 | 9.808 | 1.00 | 20.27 |
| ATOM | 3779 | CA | VAL | B | 158 | 16.314 | 84.649 | 10.563 | 1.00 | 20.35 |
| ATOM | 3780 | CB | VAL | B | 158 | 15.452 | 85.694 | 11.311 | 1.00 | 21.37 |
| ATOM | 3781 | CG1 | VAL | B | 158 | 16.338 | 86.766 | 11.943 | 1.00 | 19.60 |
| ATOM | 3782 | CG2 | VAL | B | 158 | 14.457 | 86.336 | 10.351 | 1.00 | 18.28 |
| ATOM | 3783 | C | VAL | B | 158 | 17.180 | 83.862 | 11.537 | 1.00 | 18.97 |
| ATOM | 3784 | O | VAL | B | 158 | 18.387 | 84.016 | 11.565 | 1.00 | 23.65 |
| ATOM | 3785 | N | LYS | B | 159 | 16.551 | 82.979 | 12.299 | 1.00 | 20.72 |
| ATOM | 3786 | CA | LYS | B | 159 | 17.272 | 82.126 | 13.254 | 1.00 | 19.58 |
| ATOM | 3787 | CB | LYS | B | 159 | 16.303 | 81.133 | 13.901 | 1.00 | 17.16 |
| ATOM | 3788 | CG | LYS | B | 159 | 15.249 | 81.785 | 14.788 | 1.00 | 20.51 |
| ATOM | 3789 | CD | LYS | B | 159 | 14.393 | 80.735 | 15.487 | 1.00 | 19.90 |
| ATOM | 3790 | CE | LYS | B | 159 | 13.268 | 81.368 | 16.279 | 1.00 | 21.69 |
| ATOM | 3791 | NZ | LYS | B | 159 | 12.223 | 80.372 | 16.638 | 1.00 | 22.17 |
| ATOM | 3792 | C | LYS | B | 159 | 18.405 | 81.368 | 12.570 | 1.00 | 18.48 |
| ATOM | 3793 | O | LYS | B | 159 | 19.553 | 81.433 | 13.006 | 1.00 | 17.43 |
| ATOM | 3794 | N | LEU | B | 160 | 18.065 | 80.643 | 11.482 | 1.00 | 19.66 |
| ATOM | 3795 | CA | LEU | B | 160 | 19.048 | 79.860 | 10.719 | 1.00 | 18.83 |
| ATOM | 3796 | CB | LEU | B | 160 | 18.379 | 79.163 | 9.522 | 1.00 | 19.66 |
| ATOM | 3797 | CG | LEU | B | 160 | 17.774 | 77.795 | 9.788 | 1.00 | 19.93 |
| ATOM | 3798 | CD1 | LEU | B | 160 | 16.700 | 77.486 | 8.765 | 1.00 | 21.83 |
| ATOM | 3799 | CD2 | LEU | B | 160 | 18.854 | 76.724 | 9.801 | 1.00 | 21.52 |
| ATOM | 3800 | C | LEU | B | 160 | 20.186 | 80.721 | 10.216 | 1.00 | 16.03 |
| ATOM | 3801 | O | LEU | B | 160 | 21.345 | 80.349 | 10.341 | 1.00 | 16.78 |
| ATOM | 3802 | N | TYR | B | 161 | 19.857 | 81.869 | 9.627 | 1.00 | 17.43 |
| ATOM | 3803 | CA | TYR | B | 161 | 20.888 | 82.762 | 9.089 | 1.00 | 19.10 |
| ATOM | 3804 | CB | TYR | B | 161 | 20.250 | 83.920 | 8.284 | 1.00 | 19.88 |
| ATOM | 3805 | CG | TYR | B | 161 | 19.476 | 83.557 | 7.024 | 1.00 | 24.99 |
| ATOM | 3806 | CD1 | TYR | B | 161 | 19.463 | 82.271 | 6.496 | 1.00 | 25.12 |
| ATOM | 3807 | CE1 | TYR | B | 161 | 18.725 | 81.975 | 5.358 | 1.00 | 29.18 |
| ATOM | 3808 | CD2 | TYR | B | 161 | 18.735 | 84.530 | 6.373 | 1.00 | 26.00 |
| ATOM | 3809 | CE2 | TYR | B | 161 | 18.004 | 84.249 | 5.241 | 1.00 | 29.30 |
| ATOM | 3810 | CZ | TYR | B | 161 | 17.998 | 82.976 | 4.737 | 1.00 | 30.29 |
| ATOM | 3811 | OH | TYR | B | 161 | 17.254 | 82.700 | 3.612 | 1.00 | 33.10 |
| ATOM | 3812 | C | TYR | B | 161 | 21.740 | 83.366 | 10.221 | 1.00 | 18.94 |
| ATOM | 3813 | O | TYR | B | 161 | 22.974 | 83.354 | 10.182 | 1.00 | 17.38 |
| ATOM | 3814 | N | MET | B | 162 | 21.061 | 83.926 | 11.221 | 1.00 | 18.27 |
| ATOM | 3815 | CA | MET | B | 162 | 21.767 | 84.560 | 12.347 | 1.00 | 18.73 |
| ATOM | 3816 | CB | MET | B | 162 | 20.770 | 85.237 | 13.288 | 1.00 | 16.64 |
| ATOM | 3817 | CG | MET | B | 162 | 20.235 | 86.526 | 12.724 | 1.00 | 16.75 |
| ATOM | 3818 | SD | MET | B | 162 | 21.522 | 87.547 | 11.984 | 1.00 | 21.72 |
| ATOM | 3819 | CE | MET | B | 162 | 22.654 | 87.807 | 13.364 | 1.00 | 22.96 |
| ATOM | 3820 | C | MET | B | 162 | 22.656 | 83.584 | 13.078 | 1.00 | 13.97 |
| ATOM | 3821 | O | MET | B | 162 | 23.784 | 83.906 | 13.407 | 1.00 | 18.33 |
| ATOM | 3822 | N | TYR | B | 163 | 22.146 | 82.382 | 13.301 | 1.00 | 16.83 |
| ATOM | 3823 | CA | TYR | B | 163 | 22.891 | 81.317 | 13.994 | 1.00 | 17.57 |
| ATOM | 3824 | CB | TYR | B | 163 | 21.998 | 80.075 | 14.143 | 1.00 | 16.83 |
| ATOM | 3825 | CG | TYR | B | 163 | 22.689 | 78.913 | 14.842 | 1.00 | 18.42 |
| ATOM | 3826 | CD1 | TYR | B | 163 | 22.814 | 78.874 | 16.240 | 1.00 | 16.94 |
| ATOM | 3827 | CE1 | TYR | B | 163 | 23.444 | 77.815 | 16.873 | 1.00 | 17.73 |
| ATOM | 3828 | CD2 | TYR | B | 163 | 23.214 | 77.862 | 14.106 | 1.00 | 16.91 |
| ATOM | 3829 | CE2 | TYR | B | 163 | 23.842 | 76.804 | 14.718 | 1.00 | 19.62 |
| ATOM | 3830 | CZ | TYR | B | 163 | 23.957 | 76.780 | 16.109 | 1.00 | 22.45 |
| ATOM | 3831 | OH | TYR | B | 163 | 24.585 | 75.723 | 16.715 | 1.00 | 19.94 |
| ATOM | 3832 | C | TYR | B | 163 | 24.183 | 80.938 | 13.281 | 1.00 | 18.55 |
| ATOM | 3833 | O | TYR | B | 163 | 25.267 | 80.837 | 13.907 | 1.00 | 18.64 |
| ATOM | 3834 | N | GLN | B | 164 | 24.077 | 80.716 | 11.958 | 1.00 | 17.45 |
| ATOM | 3835 | CA | GLN | B | 164 | 25.239 | 80.342 | 11.166 | 1.00 | 13.31 |
| ATOM | 3836 | CB | GLN | B | 164 | 24.817 | 79.910 | 9.762 | 1.00 | 15.13 |
| ATOM | 3837 | CG | GLN | B | 164 | 24.007 | 78.628 | 9.747 | 1.00 | 16.53 |
| ATOM | 3838 | CD | GLN | B | 164 | 23.455 | 78.316 | 8.367 | 1.00 | 16.29 |
| ATOM | 3839 | OE1 | GLN | B | 164 | 24.142 | 77.720 | 7.550 | 1.00 | 16.87 |
| ATOM | 3840 | NE2 | GLN | B | 164 | 22.224 | 78.727 | 8.104 | 1.00 | 13.57 |
| ATOM | 3841 | C | GLN | B | 164 | 26.239 | 81.472 | 11.131 | 1.00 | 9.92 |
| ATOM | 3842 | O | GLN | B | 164 | 27.430 | 81.241 | 11.174 | 1.00 | 12.36 |
| ATOM | 3843 | N | LEU | B | 165 | 25.733 | 82.703 | 11.107 | 1.00 | 11.26 |
| ATOM | 3844 | CA | LEU | B | 165 | 26.581 | 83.890 | 11.129 | 1.00 | 13.08 |
| ATOM | 3845 | CB | LEU | B | 165 | 25.702 | 85.138 | 10.991 | 1.00 | 12.81 |
| ATOM | 3846 | CG | LEU | B | 165 | 26.346 | 86.410 | 10.423 | 1.00 | 15.34 |
| ATOM | 3847 | CD1 | LEU | B | 165 | 25.759 | 87.659 | 11.062 | 1.00 | 12.66 |
| ATOM | 3848 | CD2 | LEU | B | 165 | 27.854 | 86.395 | 10.525 | 1.00 | 18.57 |
| ATOM | 3849 | C | LEU | B | 165 | 27.366 | 83.950 | 12.456 | 1.00 | 12.95 |
| ATOM | 3850 | O | LEU | B | 165 | 28.573 | 84.144 | 12.467 | 1.00 | 16.32 |
| ATOM | 3851 | N | PHE | B | 166 | 26.676 | 83.768 | 13.585 | 1.00 | 14.47 |
| ATOM | 3852 | CA | PHE | B | 166 | 27.375 | 83.790 | 14.876 | 1.00 | 12.97 |
| ATOM | 3853 | CB | PHE | B | 166 | 26.386 | 83.728 | 16.043 | 1.00 | 11.30 |
| ATOM | 3854 | CG | PHE | B | 166 | 25.697 | 85.037 | 16.242 | 1.00 | 6.37 |
| ATOM | 3855 | CD1 | PHE | B | 166 | 24.333 | 85.121 | 16.220 | 1.00 | 6.85 |
| ATOM | 3856 | CD2 | PHE | B | 166 | 26.429 | 86.183 | 16.440 | 1.00 | 7.66 |
| ATOM | 3857 | CE1 | PHE | B | 166 | 23.695 | 86.326 | 16.391 | 1.00 | 5.07 |
| ATOM | 3858 | CE2 | PHE | B | 166 | 25.800 | 87.399 | 16.616 | 1.00 | 9.58 |
| ATOM | 3859 | CZ | PHE | B | 166 | 24.428 | 87.466 | 16.589 | 1.00 | 7.06 |
| ATOM | 3860 | C | PHE | B | 166 | 28.418 | 82.689 | 14.920 | 1.00 | 10.95 |
| ATOM | 3861 | O | PHE | B | 166 | 29.570 | 82.930 | 15.309 | 1.00 | 16.03 |
| ATOM | 3862 | N | ARG | B | 167 | 28.039 | 81.497 | 14.462 | 1.00 | 12.53 |
| ATOM | 3863 | CA | ARG | B | 167 | 28.963 | 80.364 | 14.400 | 1.00 | 13.60 |
| ATOM | 3864 | CB | ARG | B | 167 | 28.275 | 79.162 | 13.750 | 1.00 | 18.63 |
| ATOM | 3865 | CG | ARG | B | 167 | 27.668 | 78.171 | 14.734 | 1.00 | 19.55 |
| ATOM | 3866 | CD | ARG | B | 167 | 27.437 | 76.814 | 14.088 | 1.00 | 19.56 |
| ATOM | 3867 | NE | ARG | B | 167 | 28.676 | 76.045 | 13.967 | 1.00 | 20.35 |
| ATOM | 3868 | CZ | ARG | B | 167 | 28.785 | 74.891 | 13.299 | 1.00 | 16.47 |
| ATOM | 3869 | NH1 | ARG | B | 167 | 27.737 | 74.355 | 12.692 | 1.00 | 20.44 |
| ATOM | 3870 | NH2 | ARG | B | 167 | 29.944 | 74.274 | 13.240 | 1.00 | 18.83 |
| ATOM | 3871 | C | ARG | B | 167 | 30.232 | 80.715 | 13.613 | 1.00 | 17.56 |
| ATOM | 3872 | O | ARG | B | 167 | 31.372 | 80.451 | 14.062 | 1.00 | 19.20 |
| ATOM | 3873 | N | SER | B | 168 | 30.065 | 81.324 | 12.435 | 1.00 | 17.30 |
| ATOM | 3874 | CA | SER | B | 168 | 31.246 | 81.695 | 11.657 | 1.00 | 17.33 |
| ATOM | 3875 | CB | SER | B | 168 | 30.870 | 82.151 | 10.230 | 1.00 | 15.58 |
| ATOM | 3876 | OG | SER | B | 168 | 30.353 | 83.476 | 10.201 | 1.00 | 13.88 |
| ATOM | 3877 | C | SER | B | 168 | 32.033 | 82.777 | 12.402 | 1.00 | 15.51 |
| ATOM | 3878 | O | SER | B | 168 | 33.263 | 82.845 | 12.329 | 1.00 | 16.25 |
| ATOM | 3879 | N | LEU | B | 169 | 31.316 | 83.615 | 13.137 | 1.00 | 15.49 |
| ATOM | 3880 | CA | LEU | B | 169 | 31.973 | 84.675 | 13.915 | 1.00 | 17.05 |
| ATOM | 3881 | CB | LEU | B | 169 | 30.954 | 85.677 | 14.436 | 1.00 | 17.09 |
| ATOM | 3882 | CG | LEU | B | 169 | 30.401 | 86.619 | 13.363 | 1.00 | 18.69 |
| ATOM | 3883 | CD1 | LEU | B | 169 | 29.354 | 87.569 | 13.922 | 1.00 | 13.84 |
| ATOM | 3884 | CD2 | LEU | B | 169 | 31.533 | 87.377 | 12.666 | 1.00 | 14.52 |
| ATOM | 3885 | C | LEU | B | 169 | 32.822 | 84.082 | 15.031 | 1.00 | 15.99 |
| ATOM | 3886 | O | LEU | B | 169 | 33.980 | 84.464 | 15.214 | 1.00 | 16.48 |
| ATOM | 3887 | N | ALA | B | 170 | 32.250 | 83.107 | 15.745 | 1.00 | 18.87 |
| ATOM | 3888 | CA | ALA | B | 170 | 32.943 | 82.407 | 16.845 | 1.00 | 21.00 |
| ATOM | 3889 | CB | ALA | B | 170 | 32.012 | 81.352 | 17.449 | 1.00 | 19.79 |
| ATOM | 3890 | C | ALA | B | 170 | 34.220 | 81.741 | 16.352 | 1.00 | 21.82 |
| ATOM | 3891 | O | ALA | B | 170 | 35.266 | 81.760 | 17.026 | 1.00 | 22.36 |
| ATOM | 3892 | N | TYR | B | 171 | 34.127 | 81.144 | 15.165 | 1.00 | 23.15 |
| ATOM | 3893 | CA | TYR | B | 171 | 35.256 | 80.458 | 14.558 | 1.00 | 19.25 |
| ATOM | 3894 | CB | TYR | B | 171 | 34.826 | 79.705 | 13.297 | 1.00 | 20.50 |
| ATOM | 3895 | CG | TYR | B | 171 | 35.971 | 78.969 | 12.643 | 1.00 | 18.22 |
| ATOM | 3896 | CD1 | TYR | B | 171 | 36.368 | 77.727 | 13.110 | 1.00 | 15.97 |
| ATOM | 3897 | CE1 | TYR | B | 171 | 37.410 | 77.051 | 12.521 | 1.00 | 18.29 |
| ATOM | 3898 | CD2 | TYR | B | 171 | 36.656 | 79.523 | 11.560 | 1.00 | 16.49 |
| ATOM | 3899 | CE2 | TYR | B | 171 | 37.705 | 78.849 | 10.964 | 1.00 | 15.82 |
| ATOM | 3900 | CZ | TYR | B | 171 | 38.073 | 77.619 | 11.446 | 1.00 | 18.15 |
| ATOM | 3901 | OH | TYR | B | 171 | 39.103 | 76.939 | 10.859 | 1.00 | 21.31 |
| ATOM | 3902 | C | TYR | B | 171 | 36.394 | 81.393 | 14.236 | 1.00 | 17.82 |
| ATOM | 3903 | O | TYR | B | 171 | 37.544 | 81.086 | 14.528 | 1.00 | 22.52 |
| ATOM | 3904 | N | ILE | B | 172 | 36.093 | 82.520 | 13.616 | 1.00 | 18.32 |
| ATOM | 3905 | CA | ILE | B | 172 | 37.147 | 83.471 | 13.249 | 1.00 | 18.09 |
| ATOM | 3906 | CB | ILE | B | 172 | 36.701 | 84.470 | 12.158 | 1.00 | 16.91 |
| ATOM | 3907 | CG2 | ILE | B | 172 | 36.617 | 83.787 | 10.793 | 1.00 | 18.79 |
| ATOM | 3908 | CG1 | ILE | B | 172 | 35.361 | 85.101 | 12.516 | 1.00 | 16.59 |
| ATOM | 3909 | CD1 | ILE | B | 172 | 35.267 | 86.556 | 12.121 | 1.00 | 17.41 |
| ATOM | 3910 | C | ILE | B | 172 | 37.676 | 84.232 | 14.471 | 1.00 | 17.72 |
| ATOM | 3911 | O | ILE | B | 172 | 38.894 | 84.411 | 14.624 | 1.00 | 16.48 |
| ATOM | 3912 | N | HIS | B | 173 | 36.759 | 84.664 | 15.346 | 1.00 | 18.14 |
| ATOM | 3913 | CA | HIS | B | 173 | 37.161 | 85.380 | 16.566 | 1.00 | 18.28 |
| ATOM | 3914 | CB | HIS | B | 173 | 35.939 | 85.810 | 17.381 | 1.00 | 14.98 |
| ATOM | 3915 | CG | HIS | B | 173 | 35.179 | 86.918 | 16.731 | 1.00 | 12.61 |
| ATOM | 3916 | CD2 | HIS | B | 173 | 35.510 | 87.730 | 15.691 | 1.00 | 15.00 |
| ATOM | 3917 | ND1 | HIS | B | 173 | 33.924 | 87.306 | 17.118 | 1.00 | 15.31 |
| ATOM | 3918 | CE1 | HIS | B | 173 | 33.508 | 88.305 | 16.349 | 1.00 | 13.51 |
| ATOM | 3919 | NE2 | HIS | B | 173 | 34.457 | 88.579 | 15.479 | 1.00 | 11.84 |
| ATOM | 3920 | C | HIS | B | 173 | 38.123 | 84.507 | 17.380 | 1.00 | 18.31 |
| ATOM | 3921 | O | HIS | B | 173 | 39.122 | 84.995 | 17.886 | 1.00 | 23.85 |
| ATOM | 3922 | N | SER | B | 174 | 37.845 | 83.200 | 17.436 | 1.00 | 21.25 |
| ATOM | 3923 | CA | SER | B | 174 | 38.706 | 82.222 | 18.136 | 1.00 | 21.23 |
| ATOM | 3924 | CB | SER | B | 174 | 38.120 | 80.800 | 18.017 | 1.00 | 23.92 |
| ATOM | 3925 | OG | SER | B | 174 | 38.766 | 80.026 | 17.009 | 1.00 | 29.48 |
| ATOM | 3926 | C | SER | B | 174 | 40.176 | 82.273 | 17.675 | 1.00 | 22.44 |
| ATOM | 3927 | O | SER | B | 174 | 41.083 | 81.828 | 18.388 | 1.00 | 20.91 |
| ATOM | 3928 | N | PHE | B | 175 | 40.410 | 82.829 | 16.482 | 1.00 | 22.81 |
| ATOM | 3929 | CA | PHE | B | 175 | 41.758 | 82.953 | 15.930 | 1.00 | 20.67 |
| ATOM | 3930 | CB | PHE | B | 175 | 41.740 | 82.656 | 14.418 | 1.00 | 23.12 |
| ATOM | 3931 | CG | PHE | B | 175 | 41.717 | 81.196 | 14.049 | 1.00 | 21.93 |
| ATOM | 3932 | CD1 | PHE | B | 175 | 42.868 | 80.436 | 14.112 | 1.00 | 21.49 |
| ATOM | 3933 | CD2 | PHE | B | 175 | 40.541 | 80.593 | 13.633 | 1.00 | 22.75 |
| ATOM | 3934 | CE1 | PHE | B | 175 | 42.847 | 79.097 | 13.770 | 1.00 | 24.14 |
| ATOM | 3935 | CE2 | PHE | B | 175 | 40.512 | 79.257 | 13.293 | 1.00 | 21.54 |
| ATOM | 3936 | CZ | PHE | B | 175 | 41.666 | 78.507 | 13.362 | 1.00 | 20.39 |
| ATOM | 3937 | C | PHE | B | 175 | 42.299 | 84.367 | 16.144 | 1.00 | 20.00 |
| ATOM | 3938 | O | PHE | B | 175 | 43.457 | 84.645 | 15.849 | 1.00 | 21.75 |
| ATOM | 3939 | N | GLY | B | 176 | 41.442 | 85.265 | 16.624 | 1.00 | 18.95 |
| ATOM | 3940 | CA | GLY | B | 176 | 41.837 | 86.654 | 16.833 | 1.00 | 20.05 |
| ATOM | 3941 | C | GLY | B | 176 | 41.498 | 87.512 | 15.617 | 1.00 | 19.66 |
| ATOM | 3942 | O | GLY | B | 176 | 41.796 | 88.705 | 15.568 | 1.00 | 17.74 |
| ATOM | 3943 | N | ILE | B | 177 | 40.885 | 86.877 | 14.626 | 1.00 | 19.43 |
| ATOM | 3944 | CA | ILE | B | 177 | 40.521 | 87.540 | 13.388 | 1.00 | 17.66 |
| ATOM | 3945 | CB | ILE | B | 177 | 40.471 | 86.528 | 12.218 | 1.00 | 18.89 |
| ATOM | 3946 | CG2 | ILE | B | 177 | 39.866 | 87.169 | 10.970 | 1.00 | 22.27 |
| ATOM | 3947 | CG1 | ILE | B | 177 | 41.864 | 85.976 | 11.924 | 1.00 | 18.79 |
| ATOM | 3948 | CD1 | ILE | B | 177 | 41.850 | 84.580 | 11.333 | 1.00 | 18.07 |
| ATOM | 3949 | C | ILE | B | 177 | 39.186 | 88.252 | 13.460 | 1.00 | 13.18 |
| ATOM | 3950 | O | ILE | B | 177 | 38.154 | 87.652 | 13.694 | 1.00 | 14.34 |
| ATOM | 3951 | N | CYS | B | 178 | 39.212 | 89.529 | 13.163 | 1.00 | 14.13 |
| ATOM | 3952 | CA | CYS | B | 178 | 38.003 | 90.313 | 13.097 | 1.00 | 15.75 |
| ATOM | 3953 | CB | CYS | B | 178 | 38.207 | 91.633 | 13.822 | 1.00 | 12.82 |
| ATOM | 3954 | SG | CYS | B | 178 | 36.704 | 92.568 | 14.016 | 1.00 | 20.44 |
| ATOM | 3955 | C | CYS | B | 178 | 37.693 | 90.561 | 11.592 | 1.00 | 16.81 |
| ATOM | 3956 | O | CYS | B | 178 | 38.616 | 90.797 | 10.812 | 1.00 | 18.44 |
| ATOM | 3957 | N | HIS | B | 179 | 36.420 | 90.470 | 11.211 | 1.00 | 17.46 |
| ATOM | 3958 | CA | HIS | B | 179 | 35.973 | 90.648 | 9.823 | 1.00 | 18.34 |
| ATOM | 3959 | CB | HIS | B | 179 | 34.557 | 90.106 | 9.660 | 1.00 | 16.84 |
| ATOM | 3960 | CG | HIS | B | 179 | 34.198 | 89.789 | 8.231 | 1.00 | 18.96 |
| ATOM | 3961 | CD2 | HIS | B | 179 | 34.490 | 88.717 | 7.451 | 1.00 | 18.32 |
| ATOM | 3962 | ND1 | HIS | B | 179 | 33.444 | 90.624 | 7.445 | 1.00 | 19.17 |
| ATOM | 3963 | CE1 | HIS | B | 179 | 33.280 | 90.087 | 6.253 | 1.00 | 16.74 |
| ATOM | 3964 | NE2 | HIS | B | 179 | 33.904 | 88.933 | 6.230 | 1.00 | 15.67 |
| ATOM | 3965 | C | HIS | B | 179 | 36.030 | 92.120 | 9.419 | 1.00 | 20.37 |
| ATOM | 3966 | O | HIS | B | 179 | 36.783 | 92.495 | 8.506 | 1.00 | 19.08 |
| ATOM | 3967 | N | ARG | B | 180 | 35.258 | 92.938 | 10.153 | 1.00 | 19.56 |
| ATOM | 3968 | CA | ARG | B | 180 | 35.198 | 94.388 | 9.981 | 1.00 | 19.52 |
| ATOM | 3969 | CB | ARG | B | 180 | 36.596 | 94.975 | 9.873 | 1.00 | 21.91 |
| ATOM | 3970 | CG | ARG | B | 180 | 37.585 | 94.396 | 10.859 | 1.00 | 18.09 |
| ATOM | 3971 | CD | ARG | B | 180 | 38.672 | 95.403 | 11.177 | 1.00 | 21.06 |
| ATOM | 3972 | NE | ARG | B | 180 | 39.531 | 95.661 | 10.019 | 1.00 | 22.36 |
| ATOM | 3973 | CZ | ARG | B | 180 | 40.540 | 96.527 | 10.013 | 1.00 | 19.95 |
| ATOM | 3974 | NH1 | ARG | B | 180 | 40.716 | 97.382 | 11.028 | 1.00 | 22.70 |
| ATOM | 3975 | NH2 | ARG | B | 180 | 41.369 | 96.553 | 8.981 | 1.00 | 18.96 |
| ATOM | 3976 | C | ARG | B | 180 | 34.323 | 94.837 | 8.799 | 1.00 | 20.34 |
| ATOM | 3977 | O | ARG | B | 180 | 34.320 | 96.016 | 8.432 | 1.00 | 20.98 |
| ATOM | 3978 | N | ASP | B | 181 | 33.546 | 93.923 | 8.246 | 1.00 | 21.43 |
| ATOM | 3979 | CA | ASP | B | 181 | 32.651 | 94.257 | 7.150 | 1.00 | 23.13 |
| ATOM | 3980 | CB | ASP | B | 181 | 33.397 | 94.205 | 5.814 | 1.00 | 22.99 |
| ATOM | 3981 | CG | ASP | B | 181 | 32.706 | 94.987 | 4.716 | 1.00 | 24.00 |
| ATOM | 3982 | OD1 | ASP | B | 181 | 31.738 | 95.710 | 5.017 | 1.00 | 22.68 |
| ATOM | 3983 | OD2 | ASP | B | 181 | 33.143 | 94.873 | 3.546 | 1.00 | 26.09 |
| ATOM | 3984 | C | ASP | B | 181 | 31.431 | 93.346 | 7.126 | 1.00 | 24.10 |
| ATOM | 3985 | O | ASP | B | 181 | 31.030 | 92.837 | 6.071 | 1.00 | 27.13 |
| ATOM | 3986 | N | ILE | B | 182 | 30.831 | 93.151 | 8.291 | 1.00 | 22.83 |
| ATOM | 3987 | CA | ILE | B | 182 | 29.655 | 92.317 | 8.422 | 1.00 | 20.44 |
| ATOM | 3988 | CB | ILE | B | 182 | 29.466 | 91.833 | 9.872 | 1.00 | 21.37 |
| ATOM | 3989 | CG2 | ILE | B | 182 | 28.127 | 91.131 | 10.053 | 1.00 | 21.22 |
| ATOM | 3990 | CG1 | ILE | B | 182 | 30.621 | 90.919 | 10.273 | 1.00 | 19.44 |
| ATOM | 3991 | CD1 | ILE | B | 182 | 30.463 | 89.488 | 9.800 | 1.00 | 19.22 |
| ATOM | 3992 | C | ILE | B | 182 | 28.421 | 93.068 | 7.972 | 1.00 | 20.80 |
| ATOM | 3993 | O | ILE | B | 182 | 28.086 | 94.108 | 8.523 | 1.00 | 20.95 |
| ATOM | 3994 | N | LYS | B | 183 | 27.767 | 92.524 | 6.941 | 1.00 | 19.52 |
| ATOM | 3995 | CA | LYS | B | 183 | 26.573 | 93.121 | 6.356 | 1.00 | 16.61 |
| ATOM | 3996 | CB | LYS | B | 183 | 26.971 | 94.319 | 5.515 | 1.00 | 15.49 |
| ATOM | 3997 | CG | LYS | B | 183 | 28.126 | 94.032 | 4.573 | 1.00 | 15.33 |
| ATOM | 3998 | CD | LYS | B | 183 | 28.436 | 95.248 | 3.726 | 1.00 | 17.88 |
| ATOM | 3999 | CE | LYS | B | 183 | 29.187 | 94.884 | 2.460 | 1.00 | 17.85 |
| ATOM | 4000 | NZ | LYS | B | 183 | 29.949 | 96.042 | 1.927 | 1.00 | 19.34 |
| ATOM | 4001 | C | LYS | B | 183 | 25.826 | 92.079 | 5.515 | 1.00 | 14.55 |
| ATOM | 4002 | O | LYS | B | 183 | 26.390 | 91.051 | 5.147 | 1.00 | 13.32 |
| ATOM | 4003 | N | PRO | B | 184 | 24.546 | 92.306 | 5.234 | 1.00 | 13.82 |
| ATOM | 4004 | CD | PRO | B | 184 | 23.783 | 93.487 | 5.647 | 1.00 | 13.63 |
| ATOM | 4005 | CA | PRO | B | 184 | 23.719 | 91.351 | 4.478 | 1.00 | 16.50 |
| ATOM | 4006 | CB | PRO | B | 184 | 22.386 | 92.079 | 4.305 | 1.00 | 14.92 |
| ATOM | 4007 | CG | PRO | B | 184 | 22.357 | 93.062 | 5.429 | 1.00 | 15.66 |
| ATOM | 4008 | C | PRO | B | 184 | 24.310 | 90.922 | 3.112 | 1.00 | 17.86 |
| ATOM | 4009 | O | PRO | B | 184 | 24.079 | 89.804 | 2.674 | 1.00 | 21.42 |
| ATOM | 4010 | N | GLN | B | 185 | 25.066 | 91.791 | 2.449 | 1.00 | 18.44 |
| ATOM | 4011 | CA | GLN | B | 185 | 25.639 | 91.416 | 1.158 | 1.00 | 21.79 |
| ATOM | 4012 | CB | GLN | B | 185 | 26.030 | 92.638 | 0.309 | 1.00 | 24.61 |
| ATOM | 4013 | CG | GLN | B | 185 | 26.490 | 93.856 | 1.102 | 1.00 | 32.20 |
| ATOM | 4014 | CD | GLN | B | 185 | 25.342 | 94.744 | 1.579 | 1.00 | 32.15 |
| ATOM | 4015 | OE1 | GLN | B | 185 | 24.883 | 95.637 | 0.862 | 1.00 | 33.73 |
| ATOM | 4016 | NE2 | GLN | B | 185 | 24.881 | 94.505 | 2.795 | 1.00 | 28.11 |
| ATOM | 4017 | C | GLN | B | 185 | 26.802 | 90.437 | 1.306 | 1.00 | 21.53 |
| ATOM | 4018 | O | GLN | B | 185 | 27.117 | 89.683 | 0.379 | 1.00 | 16.27 |
| ATOM | 4019 | N | ASN | B | 186 | 27.452 | 90.450 | 2.483 | 1.00 | 19.98 |
| ATOM | 4020 | CA | ASN | B | 186 | 28.580 | 89.570 | 2.713 | 1.00 | 16.29 |
| ATOM | 4021 | CB | ASN | B | 186 | 29.722 | 90.269 | 3.460 | 1.00 | 19.11 |
| ATOM | 4022 | CG | ASN | B | 186 | 30.457 | 91.254 | 2.549 | 1.00 | 22.13 |
| ATOM | 4023 | OD1 | ASN | B | 186 | 30.227 | 91.277 | 1.334 | 1.00 | 21.39 |
| ATOM | 4024 | ND2 | ASN | B | 186 | 31.353 | 92.064 | 3.117 | 1.00 | 19.64 |
| ATOM | 4025 | C | ASN | B | 186 | 28.178 | 88.230 | 3.283 | 1.00 | 11.95 |
| ATOM | 4026 | O | ASN | B | 186 | 29.030 | 87.417 | 3.624 | 1.00 | 15.32 |
| ATOM | 4027 | N | LEU | B | 187 | 26.875 | 87.998 | 3.326 | 1.00 | 13.26 |
| ATOM | 4028 | CA | LEU | B | 187 | 26.277 | 86.738 | 3.794 | 1.00 | 17.80 |
| ATOM | 4029 | CB | LEU | B | 187 | 25.173 | 87.001 | 4.838 | 1.00 | 16.02 |
| ATOM | 4030 | CG | LEU | B | 187 | 25.565 | 87.835 | 6.061 | 1.00 | 18.73 |
| ATOM | 4031 | CD1 | LEU | B | 187 | 24.335 | 88.161 | 6.902 | 1.00 | 14.44 |
| ATOM | 4032 | CD2 | LEU | B | 187 | 26.621 | 87.103 | 6.875 | 1.00 | 15.90 |
| ATOM | 4033 | C | LEU | B | 187 | 25.650 | 85.977 | 2.609 | 1.00 | 20.22 |
| ATOM | 4034 | O | LEU | B | 187 | 24.612 | 86.383 | 2.070 | 1.00 | 22.29 |
| ATOM | 4035 | N | LEU | B | 188 | 26.281 | 84.882 | 2.220 | 1.00 | 23.27 |
| ATOM | 4036 | CA | LEU | B | 188 | 25.803 | 84.063 | 1.112 | 1.00 | 25.98 |
| ATOM | 4037 | CB | LEU | B | 188 | 26.983 | 83.456 | 0.343 | 1.00 | 24.19 |
| ATOM | 4038 | CG | LEU | B | 188 | 28.091 | 84.426 | −0.041 | 1.00 | 22.57 |
| ATOM | 4039 | CD1 | LEU | B | 188 | 29.300 | 83.683 | −0.584 | 1.00 | 23.24 |
| ATOM | 4040 | CD2 | LEU | B | 188 | 27.582 | 85.467 | −1.024 | 1.00 | 23.33 |
| ATOM | 4041 | C | LEU | B | 188 | 24.909 | 82.949 | 1.596 | 1.00 | 28.54 |
| ATOM | 4042 | O | LEU | B | 188 | 25.114 | 82.411 | 2.687 | 1.00 | 31.17 |
| ATOM | 4043 | N | LEU | B | 189 | 23.934 | 82.590 | 0.768 | 1.00 | 29.77 |
| ATOM | 4044 | CA | LEU | B | 189 | 23.025 | 81.517 | 1.098 | 1.00 | 30.54 |
| ATOM | 4045 | CB | LEU | B | 189 | 21.679 | 82.046 | 1.587 | 1.00 | 30.11 |
| ATOM | 4046 | CG | LEU | B | 189 | 21.668 | 83.354 | 2.349 | 1.00 | 27.86 |
| ATOM | 4047 | CD1 | LEU | B | 189 | 20.296 | 84.000 | 2.226 | 1.00 | 28.53 |
| ATOM | 4048 | CD2 | LEU | B | 189 | 22.030 | 83.105 | 3.810 | 1.00 | 29.39 |
| ATOM | 4049 | C | LEU | B | 189 | 22.749 | 80.615 | −0.095 | 1.00 | 33.95 |
| ATOM | 4050 | O | LEU | B | 189 | 22.855 | 81.023 | −1.261 | 1.00 | 32.29 |
| ATOM | 4051 | N | ASP | B | 190 | 22.306 | 79.412 | 0.237 | 1.00 | 36.30 |
| ATOM | 4052 | CA | ASP | B | 190 | 21.893 | 78.423 | −0.726 | 1.00 | 38.55 |
| ATOM | 4053 | CB | ASP | B | 190 | 22.470 | 77.046 | −0.364 | 1.00 | 39.02 |
| ATOM | 4054 | CG | ASP | B | 190 | 22.338 | 76.020 | −1.487 | 1.00 | 40.50 |
| ATOM | 4055 | OD1 | ASP | B | 190 | 23.107 | 75.033 | −1.486 | 1.00 | 39.94 |
| ATOM | 4056 | OD2 | ASP | B | 190 | 21.459 | 76.197 | −2.360 | 1.00 | 41.85 |
| ATOM | 4057 | C | ASP | B | 190 | 20.362 | 78.387 | −0.691 | 1.00 | 40.13 |
| ATOM | 4058 | O | ASP | B | 190 | 19.772 | 78.016 | 0.334 | 1.00 | 38.56 |
| ATOM | 4059 | N | PRO | B | 191 | 19.697 | 78.791 | −1.804 | 1.00 | 41.84 |
| ATOM | 4060 | CD | PRO | B | 191 | 20.326 | 79.259 | −3.057 | 1.00 | 43.11 |
| ATOM | 4061 | CA | PRO | B | 191 | 18.229 | 78.814 | −1.894 | 1.00 | 43.03 |
| ATOM | 4062 | CB | PRO | B | 191 | 17.996 | 78.792 | −3.405 | 1.00 | 43.46 |
| ATOM | 4063 | CG | PRO | B | 191 | 19.154 | 79.557 | −3.957 | 1.00 | 43.83 |
| ATOM | 4064 | C | PRO | B | 191 | 17.635 | 77.579 | −1.236 | 1.00 | 42.98 |
| ATOM | 4065 | O | PRO | B | 191 | 16.719 | 77.665 | −0.426 | 1.00 | 44.87 |
| ATOM | 4066 | N | ASP | B | 192 | 18.217 | 76.442 | −1.558 | 1.00 | 43.24 |
| ATOM | 4067 | CA | ASP | B | 192 | 17.829 | 75.169 | −0.982 | 1.00 | 42.75 |
| ATOM | 4068 | CB | ASP | B | 192 | 17.737 | 74.090 | −2.079 | 1.00 | 43.62 |
| ATOM | 4069 | CG | ASP | B | 192 | 17.232 | 74.624 | −3.418 | 1.00 | 44.29 |
| ATOM | 4070 | OD1 | ASP | B | 192 | 16.060 | 75.053 | −3.487 | 1.00 | 43.96 |
| ATOM | 4071 | OD2 | ASP | B | 192 | 18.011 | 74.613 | −4.397 | 1.00 | 46.05 |
| ATOM | 4072 | C | ASP | B | 192 | 18.909 | 74.808 | 0.035 | 1.00 | 42.02 |
| ATOM | 4073 | O | ASP | B | 192 | 20.044 | 75.256 | −0.100 | 1.00 | 42.79 |
| ATOM | 4074 | N | THR | B | 193 | 18.570 | 74.035 | 1.056 | 1.00 | 39.71 |
| ATOM | 4075 | CA | THR | B | 193 | 19.551 | 73.668 | 2.091 | 1.00 | 38.55 |
| ATOM | 4076 | CB | THR | B | 193 | 20.934 | 73.269 | 1.519 | 1.00 | 38.09 |
| ATOM | 4077 | OG1 | THR | B | 193 | 21.839 | 74.363 | 1.479 | 1.00 | 37.67 |
| ATOM | 4078 | CG2 | THR | B | 193 | 20.899 | 72.623 | 0.148 | 1.00 | 39.30 |
| ATOM | 4079 | C | THR | B | 193 | 19.679 | 74.755 | 3.174 | 1.00 | 36.88 |
| ATOM | 4080 | O | THR | B | 193 | 20.018 | 74.452 | 4.312 | 1.00 | 37.44 |
| ATOM | 4081 | N | ALA | B | 194 | 19.381 | 76.008 | 2.811 | 1.00 | 34.97 |
| ATOM | 4082 | CA | ALA | B | 194 | 19.426 | 77.149 | 3.739 | 1.00 | 33.98 |
| ATOM | 4083 | CB | ALA | B | 194 | 18.427 | 76.970 | 4.874 | 1.00 | 34.13 |
| ATOM | 4084 | C | ALA | B | 194 | 20.814 | 77.419 | 4.307 | 1.00 | 33.03 |
| ATOM | 4085 | O | ALA | B | 194 | 20.951 | 78.183 | 5.266 | 1.00 | 33.38 |
| ATOM | 4086 | N | VAL | B | 195 | 21.839 | 76.811 | 3.718 | 1.00 | 29.95 |
| ATOM | 4087 | CA | VAL | B | 195 | 23.201 | 77.016 | 4.182 | 1.00 | 25.72 |
| ATOM | 4088 | CB | VAL | B | 195 | 24.183 | 76.067 | 3.461 | 1.00 | 25.44 |
| ATOM | 4089 | CG1 | VAL | B | 195 | 25.621 | 76.564 | 3.537 | 1.00 | 24.29 |
| ATOM | 4090 | CG2 | VAL | B | 195 | 24.068 | 74.657 | 4.022 | 1.00 | 21.03 |
| ATOM | 4091 | C | VAL | B | 195 | 23.620 | 78.476 | 3.999 | 1.00 | 25.93 |
| ATOM | 4092 | O | VAL | B | 195 | 23.219 | 79.125 | 3.023 | 1.00 | 26.83 |
| ATOM | 4093 | N | LEU | B | 196 | 24.420 | 78.985 | 4.950 | 1.00 | 22.33 |
| ATOM | 4094 | CA | LEU | B | 196 | 24.908 | 80.362 | 4.917 | 1.00 | 18.44 |
| ATOM | 4095 | CB | LEU | B | 196 | 24.288 | 81.214 | 6.042 | 1.00 | 21.00 |
| ATOM | 4096 | CG | LEU | B | 196 | 24.668 | 82.713 | 6.079 | 1.00 | 19.92 |
| ATOM | 4097 | CD1 | LEU | B | 196 | 23.587 | 83.533 | 6.760 | 1.00 | 19.76 |
| ATOM | 4098 | CD2 | LEU | B | 196 | 26.010 | 82.931 | 6.765 | 1.00 | 21.62 |
| ATOM | 4099 | C | LEU | B | 196 | 26.412 | 80.376 | 5.025 | 1.00 | 18.31 |
| ATOM | 4100 | O | LEU | B | 196 | 27.000 | 79.602 | 5.776 | 1.00 | 18.56 |
| ATOM | 4101 | N | LYS | B | 197 | 27.048 | 81.258 | 4.278 | 1.00 | 17.76 |
| ATOM | 4102 | CA | LYS | B | 197 | 28.486 | 81.361 | 4.322 | 1.00 | 16.84 |
| ATOM | 4103 | CB | LYS | B | 197 | 29.134 | 80.556 | 3.207 | 1.00 | 20.50 |
| ATOM | 4104 | CG | LYS | B | 197 | 28.966 | 79.050 | 3.363 | 1.00 | 19.83 |
| ATOM | 4105 | CD | LYS | B | 197 | 29.577 | 78.316 | 2.188 | 1.00 | 21.22 |
| ATOM | 4106 | CE | LYS | B | 197 | 29.875 | 76.872 | 2.525 | 1.00 | 23.66 |
| ATOM | 4107 | NZ | LYS | B | 197 | 29.618 | 75.983 | 1.362 | 1.00 | 25.07 |
| ATOM | 4108 | C | LYS | B | 197 | 28.918 | 82.804 | 4.260 | 1.00 | 18.70 |
| ATOM | 4109 | O | LYS | B | 197 | 28.379 | 83.601 | 3.473 | 1.00 | 20.29 |
| ATOM | 4110 | N | LEU | B | 198 | 29.884 | 83.124 | 5.126 | 1.00 | 19.30 |
| ATOM | 4111 | CA | LEU | B | 198 | 30.468 | 84.440 | 5.282 | 1.00 | 18.67 |
| ATOM | 4112 | CB | LEU | B | 198 | 31.199 | 84.487 | 6.643 | 1.00 | 19.83 |
| ATOM | 4113 | CG | LEU | B | 198 | 31.049 | 85.752 | 7.499 | 1.00 | 19.27 |
| ATOM | 4114 | CD1 | LEU | B | 198 | 32.200 | 85.856 | 8.496 | 1.00 | 15.42 |
| ATOM | 4115 | CD2 | LEU | B | 198 | 30.983 | 86.993 | 6.634 | 1.00 | 12.11 |
| ATOM | 4116 | C | LEU | B | 198 | 31.497 | 84.641 | 4.186 | 1.00 | 18.53 |
| ATOM | 4117 | O | LEU | B | 198 | 32.247 | 83.721 | 3.866 | 1.00 | 16.63 |
| ATOM | 4118 | N | CYS | B | 199 | 31.545 | 85.837 | 3.611 | 1.00 | 18.83 |
| ATOM | 4119 | CA | CYS | B | 199 | 32.509 | 86.077 | 2.551 | 1.00 | 19.58 |
| ATOM | 4120 | CB | CYS | B | 199 | 31.850 | 85.881 | 1.176 | 1.00 | 14.83 |
| ATOM | 4121 | SG | CYS | B | 199 | 30.667 | 87.171 | 0.756 | 1.00 | 21.79 |
| ATOM | 4122 | C | CYS | B | 199 | 33.114 | 87.461 | 2.646 | 1.00 | 19.09 |
| ATOM | 4123 | O | CYS | B | 199 | 32.754 | 88.250 | 3.519 | 1.00 | 18.83 |
| ATOM | 4124 | N | ASP | B | 200 | 34.040 | 87.729 | 1.713 | 1.00 | 18.38 |
| ATOM | 4125 | CA | ASP | B | 200 | 34.745 | 88.981 | 1.604 | 1.00 | 15.12 |
| ATOM | 4126 | CB | ASP | B | 200 | 33.819 | 90.129 | 1.230 | 1.00 | 10.15 |
| ATOM | 4127 | CG | ASP | B | 200 | 34.584 | 91.400 | 0.934 | 1.00 | 9.23 |
| ATOM | 4128 | OD1 | ASP | B | 200 | 33.950 | 92.454 | 0.818 | 1.00 | 18.68 |
| ATOM | 4129 | OD2 | ASP | B | 200 | 35.824 | 91.345 | 0.819 | 1.00 | 17.92 |
| ATOM | 4130 | C | ASP | B | 200 | 35.548 | 89.290 | 2.856 | 1.00 | 18.42 |
| ATOM | 4131 | O | ASP | B | 200 | 35.082 | 89.992 | 3.757 | 1.00 | 18.47 |
| ATOM | 4132 | N | PHE | B | 201 | 36.758 | 88.756 | 2.879 | 1.00 | 19.43 |
| ATOM | 4133 | CA | PHE | B | 201 | 37.690 | 88.932 | 3.970 | 1.00 | 20.67 |
| ATOM | 4134 | CB | PHE | B | 201 | 38.359 | 87.588 | 4.287 | 1.00 | 19.84 |
| ATOM | 4135 | CG | PHE | B | 201 | 37.461 | 86.667 | 5.077 | 1.00 | 20.16 |
| ATOM | 4136 | CD1 | PHE | B | 201 | 36.402 | 86.009 | 4.467 | 1.00 | 20.40 |
| ATOM | 4137 | CD2 | PHE | B | 201 | 37.665 | 86.475 | 6.446 | 1.00 | 20.19 |
| ATOM | 4138 | CE1 | PHE | B | 201 | 35.560 | 85.184 | 5.201 | 1.00 | 21.68 |
| ATOM | 4139 | CE2 | PHE | B | 201 | 36.834 | 85.649 | 7.185 | 1.00 | 18.96 |
| ATOM | 4140 | CZ | PHE | B | 201 | 35.780 | 85.005 | 6.568 | 1.00 | 22.32 |
| ATOM | 4141 | C | PHE | B | 201 | 38.720 | 90.014 | 3.634 | 1.00 | 21.02 |
| ATOM | 4142 | O | PHE | B | 201 | 39.811 | 90.034 | 4.177 | 1.00 | 22.62 |
| ATOM | 4143 | N | GLY | B | 202 | 38.337 | 90.924 | 2.730 | 1.00 | 23.59 |
| ATOM | 4144 | CA | GLY | B | 202 | 39.206 | 92.015 | 2.313 | 1.00 | 24.29 |
| ATOM | 4145 | C | GLY | B | 202 | 39.410 | 93.093 | 3.372 | 1.00 | 23.17 |
| ATOM | 4146 | O | GLY | B | 202 | 40.401 | 93.819 | 3.342 | 1.00 | 23.40 |
| ATOM | 4147 | N | SER | B | 203 | 38.491 | 93.197 | 4.315 | 1.00 | 25.53 |
| ATOM | 4148 | CA | SER | B | 203 | 38.617 | 94.189 | 5.390 | 1.00 | 24.92 |
| ATOM | 4149 | CB | SER | B | 203 | 37.283 | 94.897 | 5.589 | 1.00 | 25.38 |
| ATOM | 4150 | OG | SER | B | 203 | 37.015 | 95.804 | 4.543 | 1.00 | 27.73 |
| ATOM | 4151 | C | SER | B | 203 | 39.006 | 93.516 | 6.708 | 1.00 | 25.45 |
| ATOM | 4152 | O | SER | B | 203 | 39.035 | 94.168 | 7.750 | 1.00 | 28.67 |
| ATOM | 4153 | N | ALA | B | 204 | 39.242 | 92.202 | 6.663 | 1.00 | 24.63 |
| ATOM | 4154 | CA | ALA | B | 204 | 39.564 | 91.396 | 7.847 | 1.00 | 24.22 |
| ATOM | 4155 | CB | ALA | B | 204 | 39.383 | 89.912 | 7.551 | 1.00 | 21.47 |
| ATOM | 4156 | C | ALA | B | 204 | 40.957 | 91.672 | 8.416 | 1.00 | 24.85 |
| ATOM | 4157 | O | ALA | B | 204 | 41.871 | 92.076 | 7.695 | 1.00 | 20.84 |
| ATOM | 4158 | N | LYS | B | 205 | 41.116 | 91.461 | 9.737 | 1.00 | 24.29 |
| ATOM | 4159 | CA | LYS | B | 205 | 42.407 | 91.716 | 10.369 | 1.00 | 24.23 |
| ATOM | 4160 | CB | LYS | B | 205 | 42.583 | 93.216 | 10.584 | 1.00 | 22.03 |
| ATOM | 4161 | CG | LYS | B | 205 | 44.029 | 93.655 | 10.548 | 1.00 | 24.61 |
| ATOM | 4162 | CD | LYS | B | 205 | 44.229 | 94.953 | 11.308 | 1.00 | 23.18 |
| ATOM | 4163 | CE | LYS | B | 205 | 45.596 | 95.010 | 11.957 | 1.00 | 24.28 |
| ATOM | 4164 | NZ | LYS | B | 205 | 46.307 | 96.273 | 11.632 | 1.00 | 29.00 |
| ATOM | 4165 | C | LYS | B | 205 | 42.601 | 90.994 | 11.704 | 1.00 | 25.96 |
| ATOM | 4166 | O | LYS | B | 205 | 41.662 | 90.788 | 12.477 | 1.00 | 25.46 |
| ATOM | 4167 | N | GLN | B | 206 | 43.860 | 90.671 | 11.972 | 1.00 | 28.37 |
| ATOM | 4168 | CA | GLN | B | 206 | 44.269 | 90.032 | 13.208 | 1.00 | 29.42 |
| ATOM | 4169 | CB | GLN | B | 206 | 45.544 | 89.230 | 12.967 | 1.00 | 32.05 |
| ATOM | 4170 | CG | GLN | B | 206 | 45.375 | 88.117 | 11.937 | 1.00 | 38.78 |
| ATOM | 4171 | CD | GLN | B | 206 | 45.021 | 86.772 | 12.554 | 1.00 | 41.77 |
| ATOM | 4172 | OE1 | GLN | B | 206 | 44.861 | 85.776 | 11.840 | 1.00 | 43.68 |
| ATOM | 4173 | NE2 | GLN | B | 206 | 44.904 | 86.731 | 13.885 | 1.00 | 42.59 |
| ATOM | 4174 | C | GLN | B | 206 | 44.514 | 91.112 | 14.259 | 1.00 | 27.78 |
| ATOM | 4175 | O | GLN | B | 206 | 45.570 | 91.719 | 14.299 | 1.00 | 27.97 |
| ATOM | 4176 | N | LEU | B | 207 | 43.508 | 91.368 | 15.077 | 1.00 | 28.77 |
| ATOM | 4177 | CA | LEU | B | 207 | 43.591 | 92.401 | 16.101 | 1.00 | 28.47 |
| ATOM | 4178 | CB | LEU | B | 207 | 42.215 | 92.671 | 16.709 | 1.00 | 27.69 |
| ATOM | 4179 | CG | LEU | B | 207 | 41.111 | 93.057 | 15.727 | 1.00 | 29.12 |
| ATOM | 4180 | CD1 | LEU | B | 207 | 39.953 | 93.708 | 16.463 | 1.00 | 27.92 |
| ATOM | 4181 | CD2 | LEU | B | 207 | 41.649 | 93.985 | 14.645 | 1.00 | 28.33 |
| ATOM | 4182 | C | LEU | B | 207 | 44.591 | 92.060 | 17.192 | 1.00 | 28.80 |
| ATOM | 4183 | O | LEU | B | 207 | 44.622 | 90.934 | 17.698 | 1.00 | 29.81 |
| ATOM | 4184 | N | VAL | B | 208 | 45.395 | 93.054 | 17.558 | 1.00 | 30.07 |
| ATOM | 4185 | CA | VAL | B | 208 | 46.393 | 92.904 | 18.614 | 1.00 | 30.14 |
| ATOM | 4186 | CB | VAL | B | 208 | 47.810 | 92.530 | 18.110 | 1.00 | 30.08 |
| ATOM | 4187 | CG1 | VAL | B | 208 | 47.763 | 91.621 | 16.885 | 1.00 | 31.56 |
| ATOM | 4188 | CG2 | VAL | B | 208 | 48.659 | 93.757 | 17.849 | 1.00 | 32.50 |
| ATOM | 4189 | C | VAL | B | 208 | 46.417 | 94.153 | 19.497 | 1.00 | 29.58 |
| ATOM | 4190 | O | VAL | B | 208 | 46.334 | 95.275 | 18.992 | 1.00 | 27.39 |
| ATOM | 4191 | N | ARG | B | 209 | 46.487 | 93.924 | 20.823 | 1.00 | 30.91 |
| ATOM | 4192 | CA | ARG | B | 209 | 46.479 | 94.991 | 21.849 | 1.00 | 31.31 |
| ATOM | 4193 | CB | ARG | B | 209 | 46.964 | 94.423 | 23.206 | 1.00 | 31.12 |
| ATOM | 4194 | CG | ARG | B | 209 | 47.860 | 95.375 | 24.011 | 1.00 | 29.38 |
| ATOM | 4195 | CD | ARG | B | 209 | 47.882 | 95.037 | 25.502 | 1.00 | 26.82 |
| ATOM | 4196 | NE | ARG | B | 209 | 47.812 | 96.245 | 26.319 | 1.00 | 21.48 |
| ATOM | 4197 | CZ | ARG | B | 209 | 46.672 | 96.767 | 26.796 | 1.00 | 23.43 |
| ATOM | 4198 | NH1 | ARG | B | 209 | 45.510 | 96.124 | 26.651 | 1.00 | 20.48 |
| ATOM | 4199 | NH2 | ARG | B | 209 | 46.698 | 97.934 | 27.434 | 1.00 | 25.12 |
| ATOM | 4200 | C | ARG | B | 209 | 47.319 | 96.213 | 21.485 | 1.00 | 31.54 |
| ATOM | 4201 | O | ARG | B | 209 | 46.815 | 97.335 | 21.453 | 1.00 | 34.08 |
| ATOM | 4202 | N | GLY | B | 210 | 48.606 | 96.004 | 21.280 | 1.00 | 32.47 |
| ATOM | 4203 | CA | GLY | B | 210 | 49.494 | 97.118 | 20.995 | 1.00 | 34.33 |
| ATOM | 4204 | C | GLY | B | 210 | 49.109 | 98.001 | 19.812 | 1.00 | 35.22 |
| ATOM | 4205 | O | GLY | B | 210 | 48.892 | 99.209 | 19.974 | 1.00 | 35.81 |
| ATOM | 4206 | N | GLU | B | 211 | 49.097 | 97.410 | 18.615 | 1.00 | 35.57 |
| ATOM | 4207 | CA | GLU | B | 211 | 48.825 | 98.140 | 17.371 | 1.00 | 32.82 |
| ATOM | 4208 | CB | GLU | B | 211 | 49.240 | 97.316 | 16.150 | 1.00 | 35.30 |
| ATOM | 4209 | CG | GLU | B | 211 | 48.588 | 95.946 | 16.085 | 1.00 | 37.01 |
| ATOM | 4210 | CD | GLU | B | 211 | 49.189 | 95.054 | 15.011 | 1.00 | 38.55 |
| ATOM | 4211 | OE1 | GLU | B | 211 | 50.326 | 94.560 | 15.212 | 1.00 | 38.76 |
| ATOM | 4212 | OE2 | GLU | B | 211 | 48.517 | 94.842 | 13.977 | 1.00 | 36.23 |
| ATOM | 4213 | C | GLU | B | 211 | 47.402 | 98.656 | 17.212 | 1.00 | 29.12 |
| ATOM | 4214 | O | GLU | B | 211 | 46.431 | 97.909 | 17.298 | 1.00 | 30.00 |
| ATOM | 4215 | N | PRO | B | 212 | 47.273 | 99.957 | 16.933 | 1.00 | 26.97 |
| ATOM | 4216 | CD | PRO | B | 212 | 48.391 | 100.908 | 16.785 | 1.00 | 26.49 |
| ATOM | 4217 | CA | PRO | B | 212 | 45.980 | 100.590 | 16.707 | 1.00 | 26.42 |
| ATOM | 4218 | CB | PRO | B | 212 | 46.305 | 102.081 | 16.805 | 1.00 | 26.02 |
| ATOM | 4219 | CG | PRO | B | 212 | 47.723 | 102.187 | 16.374 | 1.00 | 25.87 |
| ATOM | 4220 | C | PRO | B | 212 | 45.430 | 100.254 | 15.306 | 1.00 | 25.79 |
| ATOM | 4221 | O | PRO | B | 212 | 46.169 | 99.812 | 14.422 | 1.00 | 25.35 |
| ATOM | 4222 | N | ASN | B | 213 | 44.131 | 100.459 | 15.130 | 1.00 | 24.87 |
| ATOM | 4223 | CA | ASN | B | 213 | 43.445 | 100.187 | 13.875 | 1.00 | 25.11 |
| ATOM | 4224 | CB | ASN | B | 213 | 42.583 | 98.934 | 13.998 | 1.00 | 25.01 |
| ATOM | 4225 | CG | ASN | B | 213 | 43.400 | 97.703 | 14.319 | 1.00 | 27.74 |
| ATOM | 4226 | OD1 | ASN | B | 213 | 44.306 | 97.333 | 13.570 | 1.00 | 29.67 |
| ATOM | 4227 | ND2 | ASN | B | 213 | 43.092 | 97.063 | 15.443 | 1.00 | 27.23 |
| ATOM | 4228 | C | ASN | B | 213 | 42.579 | 101.370 | 13.491 | 1.00 | 24.44 |
| ATOM | 4229 | O | ASN | B | 213 | 42.169 | 102.145 | 14.355 | 1.00 | 22.83 |
| ATOM | 4230 | N | VAL | B | 214 | 42.304 | 101.515 | 12.188 | 1.00 | 24.44 |
| ATOM | 4231 | CA | VAL | B | 214 | 41.482 | 102.630 | 11.711 | 1.00 | 21.41 |
| ATOM | 4232 | CB | VAL | B | 214 | 41.536 | 102.812 | 10.165 | 1.00 | 24.20 |
| ATOM | 4233 | CG1 | VAL | B | 214 | 42.975 | 102.952 | 9.698 | 1.00 | 21.35 |
| ATOM | 4234 | CG2 | VAL | B | 214 | 40.841 | 101.659 | 9.444 | 1.00 | 20.54 |
| ATOM | 4235 | C | VAL | B | 214 | 40.041 | 102.501 | 12.192 | 1.00 | 19.64 |
| ATOM | 4236 | O | VAL | B | 214 | 39.511 | 101.399 | 12.323 | 1.00 | 20.17 |
| ATOM | 4237 | N | SER | B | 215 | 39.421 | 103.636 | 12.474 | 1.00 | 20.26 |
| ATOM | 4238 | CA | SER | B | 215 | 38.046 | 103.670 | 12.967 | 1.00 | 20.21 |
| ATOM | 4239 | CB | SER | B | 215 | 37.862 | 104.860 | 13.907 | 1.00 | 21.10 |
| ATOM | 4240 | OG | SER | B | 215 | 38.487 | 106.017 | 13.378 | 1.00 | 23.88 |
| ATOM | 4241 | C | SER | B | 215 | 36.995 | 103.698 | 11.841 | 1.00 | 20.47 |
| ATOM | 4242 | O | SER | B | 215 | 35.899 | 103.162 | 11.999 | 1.00 | 19.45 |
| ATOM | 4243 | N | PTR | B | 216 | 37.323 | 104.316 | 10.707 | 1.00 | 22.87 |
| ATOM | 4244 | CA | PTR | B | 216 | 36.378 | 104.370 | 9.582 | 1.00 | 23.82 |
| ATOM | 4245 | CB | PTR | B | 216 | 36.683 | 105.571 | 8.654 | 1.00 | 24.62 |
| ATOM | 4246 | CG | PTR | B | 216 | 35.507 | 106.017 | 7.799 | 1.00 | 24.52 |
| ATOM | 4247 | CD1 | PTR | B | 216 | 34.798 | 107.173 | 8.107 | 1.00 | 25.84 |
| ATOM | 4248 | CE1 | PTR | B | 216 | 33.722 | 107.591 | 7.327 | 1.00 | 27.22 |
| ATOM | 4249 | CD2 | PTR | B | 216 | 35.104 | 105.283 | 6.675 | 1.00 | 25.91 |
| ATOM | 4250 | CE2 | PTR | B | 216 | 34.025 | 105.691 | 5.890 | 1.00 | 27.15 |
| ATOM | 4251 | CZ | PTR | B | 216 | 33.334 | 106.847 | 6.219 | 1.00 | 26.61 |
| ATOM | 4252 | OH | PTR | B | 216 | 32.261 | 107.279 | 5.450 | 1.00 | 26.46 |
| ATOM | 4253 | C | PTR | B | 216 | 36.445 | 103.053 | 8.811 | 1.00 | 23.08 |
| ATOM | 4254 | O | PTR | B | 216 | 37.208 | 102.916 | 7.869 | 1.00 | 24.94 |
| ATOM | 4255 | P | PTR | B | 216 | 31.041 | 106.344 | 5.227 | 1.00 | 27.03 |
| ATOM | 4256 | O1P | PTR | B | 216 | 30.362 | 106.877 | 3.942 | 1.00 | 30.42 |
| ATOM | 4257 | O2P | PTR | B | 216 | 30.099 | 106.633 | 6.418 | 1.00 | 28.23 |
| ATOM | 4258 | O3P | PTR | B | 216 | 31.426 | 104.917 | 5.114 | 1.00 | 24.51 |
| ATOM | 4259 | N | ILE | B | 217 | 35.749 | 102.053 | 9.346 | 1.00 | 24.80 |
| ATOM | 4260 | CA | ILE | B | 217 | 35.641 | 100.783 | 8.656 | 1.00 | 23.98 |
| ATOM | 4261 | CB | ILE | B | 217 | 36.542 | 99.693 | 9.287 | 1.00 | 26.25 |
| ATOM | 4262 | CG2 | ILE | B | 217 | 37.778 | 99.450 | 8.438 | 1.00 | 25.26 |
| ATOM | 4263 | CG1 | ILE | B | 217 | 36.953 | 100.051 | 10.708 | 1.00 | 26.90 |
| ATOM | 4264 | CD1 | ILE | B | 217 | 37.119 | 98.845 | 11.613 | 1.00 | 27.85 |
| ATOM | 4265 | C | ILE | B | 217 | 34.195 | 100.382 | 8.691 | 1.00 | 24.40 |
| ATOM | 4266 | O | ILE | B | 217 | 33.361 | 101.080 | 9.276 | 1.00 | 26.29 |
| ATOM | 4267 | N | CYS | B | 218 | 33.911 | 99.263 | 8.073 | 1.00 | 24.77 |
| ATOM | 4268 | CA | CYS | B | 218 | 32.541 | 98.748 | 8.030 | 1.00 | 23.40 |
| ATOM | 4269 | CB | CYS | B | 218 | 31.965 | 98.740 | 9.450 | 1.00 | 23.30 |
| ATOM | 4270 | SG | CYS | B | 218 | 32.745 | 97.535 | 10.578 | 1.00 | 26.72 |
| ATOM | 4271 | C | CYS | B | 218 | 31.653 | 99.526 | 7.070 | 1.00 | 23.74 |
| ATOM | 4272 | O | CYS | B | 218 | 32.051 | 100.535 | 6.510 | 1.00 | 27.68 |
| ATOM | 4273 | N | SER | B | 219 | 30.463 | 99.027 | 6.853 | 1.00 | 22.47 |
| ATOM | 4274 | CA | SER | B | 219 | 29.481 | 99.745 | 6.088 | 1.00 | 22.56 |
| ATOM | 4275 | CB | SER | B | 219 | 28.517 | 98.836 | 5.344 | 1.00 | 16.39 |
| ATOM | 4276 | OG | SER | B | 219 | 27.433 | 99.582 | 4.804 | 1.00 | 24.70 |
| ATOM | 4277 | C | SER | B | 219 | 28.732 | 100.597 | 7.092 | 1.00 | 23.73 |
| ATOM | 4278 | O | SER | B | 219 | 28.194 | 100.091 | 8.082 | 1.00 | 26.17 |
| ATOM | 4279 | N | ARG | B | 220 | 28.678 | 101.890 | 6.835 | 1.00 | 22.97 |
| ATOM | 4280 | CA | ARG | B | 220 | 28.046 | 102.841 | 7.732 | 1.00 | 23.82 |
| ATOM | 4281 | CB | ARG | B | 220 | 27.485 | 104.019 | 6.956 | 1.00 | 22.63 |
| ATOM | 4282 | CG | ARG | B | 220 | 26.816 | 105.032 | 7.862 | 1.00 | 23.19 |
| ATOM | 4283 | CD | ARG | B | 220 | 26.068 | 106.069 | 7.070 | 1.00 | 21.58 |
| ATOM | 4284 | NE | ARG | B | 220 | 26.939 | 106.750 | 6.124 | 1.00 | 22.96 |
| ATOM | 4285 | CZ | ARG | B | 220 | 26.515 | 107.289 | 4.984 | 1.00 | 23.07 |
| ATOM | 4286 | NH1 | ARG | B | 220 | 25.240 | 107.221 | 4.634 | 1.00 | 20.87 |
| ATOM | 4287 | NH2 | ARG | B | 220 | 27.377 | 107.897 | 4.195 | 1.00 | 24.87 |
| ATOM | 4288 | C | ARG | B | 220 | 26.952 | 102.240 | 8.617 | 1.00 | 24.57 |
| ATOM | 4289 | O | ARG | B | 220 | 27.083 | 102.186 | 9.840 | 1.00 | 29.08 |
| ATOM | 4290 | N | TYR | B | 221 | 25.869 | 101.778 | 7.997 | 1.00 | 22.44 |
| ATOM | 4291 | CA | TYR | B | 221 | 24.708 | 101.225 | 8.682 | 1.00 | 21.21 |
| ATOM | 4292 | CB | TYR | B | 221 | 23.732 | 100.619 | 7.671 | 1.00 | 22.74 |
| ATOM | 4293 | CG | TYR | B | 221 | 23.028 | 101.592 | 6.747 | 1.00 | 25.19 |
| ATOM | 4294 | CD1 | TYR | B | 221 | 22.137 | 101.122 | 5.786 | 1.00 | 26.74 |
| ATOM | 4295 | CE1 | TYR | B | 221 | 21.484 | 101.981 | 4.921 | 1.00 | 29.14 |
| ATOM | 4296 | CD2 | TYR | B | 221 | 23.248 | 102.959 | 6.818 | 1.00 | 26.22 |
| ATOM | 4297 | CE2 | TYR | B | 221 | 22.599 | 103.830 | 5.960 | 1.00 | 28.68 |
| ATOM | 4298 | CZ | TYR | B | 221 | 21.717 | 103.334 | 5.011 | 1.00 | 29.87 |
| ATOM | 4299 | OH | TYR | B | 221 | 21.076 | 104.193 | 4.154 | 1.00 | 28.44 |
| ATOM | 4300 | C | TYR | B | 221 | 25.025 | 100.169 | 9.761 | 1.00 | 20.80 |
| ATOM | 4301 | O | TYR | B | 221 | 24.239 | 100.001 | 10.695 | 1.00 | 21.60 |
| ATOM | 4302 | N | TYR | B | 222 | 26.116 | 99.433 | 9.585 | 1.00 | 16.82 |
| ATOM | 4303 | CA | TYR | B | 222 | 26.512 | 98.341 | 10.477 | 1.00 | 17.54 |
| ATOM | 4304 | CB | TYR | B | 222 | 26.734 | 97.073 | 9.611 | 1.00 | 17.40 |
| ATOM | 4305 | CG | TYR | B | 222 | 25.634 | 96.916 | 8.575 | 1.00 | 19.92 |
| ATOM | 4306 | CD1 | TYR | B | 222 | 24.517 | 96.131 | 8.836 | 1.00 | 19.27 |
| ATOM | 4307 | CE1 | TYR | B | 222 | 23.469 | 96.062 | 7.943 | 1.00 | 21.70 |
| ATOM | 4308 | CD2 | TYR | B | 222 | 25.672 | 97.628 | 7.374 | 1.00 | 21.19 |
| ATOM | 4309 | CE2 | TYR | B | 222 | 24.628 | 97.549 | 6.463 | 1.00 | 20.78 |
| ATOM | 4310 | CZ | TYR | B | 222 | 23.528 | 96.770 | 6.759 | 1.00 | 21.97 |
| ATOM | 4311 | OH | TYR | B | 222 | 22.466 | 96.725 | 5.892 | 1.00 | 21.65 |
| ATOM | 4312 | C | TYR | B | 222 | 27.764 | 98.666 | 11.292 | 1.00 | 15.94 |
| ATOM | 4313 | O | TYR | B | 222 | 28.409 | 97.769 | 11.841 | 1.00 | 18.58 |
| ATOM | 4314 | N | ARG | B | 223 | 28.105 | 99.936 | 11.356 | 1.00 | 15.02 |
| ATOM | 4315 | CA | ARG | B | 223 | 29.285 | 100.393 | 12.079 | 1.00 | 17.28 |
| ATOM | 4316 | CB | ARG | B | 223 | 29.764 | 101.686 | 11.436 | 1.00 | 14.67 |
| ATOM | 4317 | CG | ARG | B | 223 | 31.143 | 102.114 | 11.865 | 1.00 | 14.12 |
| ATOM | 4318 | CD | ARG | B | 223 | 31.535 | 103.403 | 11.175 | 1.00 | 18.42 |
| ATOM | 4319 | NE | ARG | B | 223 | 32.044 | 103.122 | 9.835 | 1.00 | 20.42 |
| ATOM | 4320 | CZ | ARG | B | 223 | 31.654 | 103.746 | 8.720 | 1.00 | 20.83 |
| ATOM | 4321 | NH1 | ARG | B | 223 | 30.909 | 104.835 | 8.767 | 1.00 | 17.04 |
| ATOM | 4322 | NH2 | ARG | B | 223 | 32.062 | 103.282 | 7.546 | 1.00 | 21.59 |
| ATOM | 4323 | C | ARG | B | 223 | 28.990 | 100.634 | 13.585 | 1.00 | 16.29 |
| ATOM | 4324 | O | ARG | B | 223 | 28.024 | 101.318 | 13.934 | 1.00 | 16.79 |
| ATOM | 4325 | N | ALA | B | 224 | 29.837 | 100.063 | 14.438 | 1.00 | 17.91 |
| ATOM | 4326 | CA | ALA | B | 224 | 29.722 | 100.177 | 15.913 | 1.00 | 19.04 |
| ATOM | 4327 | CB | ALA | B | 224 | 30.712 | 99.232 | 16.570 | 1.00 | 16.46 |
| ATOM | 4328 | C | ALA | B | 224 | 29.966 | 101.607 | 16.397 | 1.00 | 18.94 |
| ATOM | 4329 | O | ALA | B | 224 | 30.870 | 102.279 | 15.916 | 1.00 | 21.70 |
| ATOM | 4330 | N | PRO | B | 225 | 29.183 | 102.079 | 17.383 | 1.00 | 21.63 |
| ATOM | 4331 | CD | PRO | B | 225 | 28.120 | 101.321 | 18.062 | 1.00 | 23.65 |
| ATOM | 4332 | CA | PRO | B | 225 | 29.315 | 103.440 | 17.954 | 1.00 | 22.23 |
| ATOM | 4333 | CB | PRO | B | 225 | 28.444 | 103.404 | 19.221 | 1.00 | 23.95 |
| ATOM | 4334 | CG | PRO | B | 225 | 28.089 | 101.965 | 19.424 | 1.00 | 22.74 |
| ATOM | 4335 | C | PRO | B | 225 | 30.743 | 103.811 | 18.315 | 1.00 | 20.39 |
| ATOM | 4336 | O | PRO | B | 225 | 31.152 | 104.962 | 18.150 | 1.00 | 22.02 |
| ATOM | 4337 | N | GLU | B | 226 | 31.501 | 102.832 | 18.797 | 1.00 | 19.06 |
| ATOM | 4338 | CA | GLU | B | 226 | 32.898 | 103.041 | 19.179 | 1.00 | 19.14 |
| ATOM | 4339 | CB | GLU | B | 226 | 33.450 | 101.789 | 19.851 | 1.00 | 18.75 |
| ATOM | 4340 | CG | GLU | B | 226 | 32.562 | 100.561 | 19.645 | 1.00 | 20.23 |
| ATOM | 4341 | CD | GLU | B | 226 | 33.352 | 99.289 | 19.603 | 1.00 | 17.38 |
| ATOM | 4342 | OE1 | GLU | B | 226 | 32.736 | 98.213 | 19.618 | 1.00 | 19.90 |
| ATOM | 4343 | OE2 | GLU | B | 226 | 34.598 | 99.368 | 19.552 | 1.00 | 24.74 |
| ATOM | 4344 | C | GLU | B | 226 | 33.746 | 103.423 | 17.963 | 1.00 | 20.82 |
| ATOM | 4345 | O | GLU | B | 226 | 34.666 | 104.248 | 18.066 | 1.00 | 20.13 |
| ATOM | 4346 | N | LEU | B | 227 | 33.408 | 102.851 | 16.800 | 1.00 | 19.41 |
| ATOM | 4347 | CA | LEU | B | 227 | 34.110 | 103.184 | 15.556 | 1.00 | 19.05 |
| ATOM | 4348 | CB | LEU | B | 227 | 33.629 | 102.290 | 14.407 | 1.00 | 16.10 |
| ATOM | 4349 | CG | LEU | B | 227 | 34.416 | 101.013 | 14.147 | 1.00 | 15.05 |
| ATOM | 4350 | CD1 | LEU | B | 227 | 35.803 | 101.042 | 14.756 | 1.00 | 12.73 |
| ATOM | 4351 | CD2 | LEU | B | 227 | 33.641 | 99.800 | 14.580 | 1.00 | 14.58 |
| ATOM | 4352 | C | LEU | B | 227 | 33.779 | 104.624 | 15.220 | 1.00 | 14.97 |
| ATOM | 4353 | O | LEU | B | 227 | 34.655 | 105.405 | 14.906 | 1.00 | 20.12 |
| ATOM | 4354 | N | ILE | B | 228 | 32.496 | 104.961 | 15.340 | 1.00 | 17.59 |
| ATOM | 4355 | CA | ILE | B | 228 | 31.993 | 106.311 | 15.103 | 1.00 | 18.11 |
| ATOM | 4356 | CB | ILE | B | 228 | 30.471 | 106.365 | 15.223 | 1.00 | 18.51 |
| ATOM | 4357 | CG2 | ILE | B | 228 | 29.947 | 107.738 | 14.812 | 1.00 | 21.29 |
| ATOM | 4358 | CG1 | ILE | B | 228 | 29.862 | 105.261 | 14.349 | 1.00 | 18.14 |
| ATOM | 4359 | CD1 | ILE | B | 228 | 28.353 | 105.347 | 14.185 | 1.00 | 16.84 |
| ATOM | 4360 | C | ILE | B | 228 | 32.678 | 107.310 | 16.049 | 1.00 | 23.42 |
| ATOM | 4361 | O | ILE | B | 228 | 32.848 | 108.489 | 15.716 | 1.00 | 24.97 |
| ATOM | 4362 | N | PHE | B | 229 | 33.167 | 106.795 | 17.182 | 1.00 | 23.68 |
| ATOM | 4363 | CA | PHE | B | 229 | 33.958 | 107.584 | 18.111 | 1.00 | 25.34 |
| ATOM | 4364 | CB | PHE | B | 229 | 33.707 | 107.151 | 19.578 | 1.00 | 22.49 |
| ATOM | 4365 | CG | PHE | B | 229 | 32.480 | 107.775 | 20.225 | 1.00 | 19.47 |
| ATOM | 4366 | CD1 | PHE | B | 229 | 31.276 | 107.093 | 20.272 | 1.00 | 17.35 |
| ATOM | 4367 | CD2 | PHE | B | 229 | 32.534 | 109.048 | 20.782 | 1.00 | 20.35 |
| ATOM | 4368 | CE1 | PHE | B | 229 | 30.157 | 107.659 | 20.852 | 1.00 | 17.59 |
| ATOM | 4369 | CE2 | PHE | B | 229 | 31.415 | 109.619 | 21.365 | 1.00 | 17.04 |
| ATOM | 4370 | CZ | PHE | B | 229 | 30.225 | 108.923 | 21.398 | 1.00 | 18.69 |
| ATOM | 4371 | C | PHE | B | 229 | 35.402 | 107.294 | 17.669 | 1.00 | 26.92 |
| ATOM | 4372 | O | PHE | B | 229 | 35.636 | 106.318 | 16.975 | 1.00 | 29.38 |
| ATOM | 4373 | N | GLY | B | 230 | 36.364 | 108.125 | 17.992 | 1.00 | 28.35 |
| ATOM | 4374 | CA | GLY | B | 230 | 37.725 | 107.851 | 17.493 | 1.00 | 26.41 |
| ATOM | 4375 | C | GLY | B | 230 | 38.364 | 106.514 | 17.891 | 1.00 | 25.16 |
| ATOM | 4376 | O | GLY | B | 230 | 39.580 | 106.387 | 17.800 | 1.00 | 24.78 |
| ATOM | 4377 | N | ALA | B | 231 | 37.577 | 105.530 | 18.348 | 1.00 | 25.97 |
| ATOM | 4378 | CA | ALA | B | 231 | 38.134 | 104.234 | 18.788 | 1.00 | 26.04 |
| ATOM | 4379 | CB | ALA | B | 231 | 37.065 | 103.275 | 19.280 | 1.00 | 26.83 |
| ATOM | 4380 | C | ALA | B | 231 | 39.050 | 103.575 | 17.767 | 1.00 | 26.99 |
| ATOM | 4381 | O | ALA | B | 231 | 38.628 | 103.158 | 16.679 | 1.00 | 27.04 |
| ATOM | 4382 | N | THR | B | 232 | 40.303 | 103.485 | 18.150 | 1.00 | 26.36 |
| ATOM | 4383 | CA | THR | B | 232 | 41.349 | 102.893 | 17.346 | 1.00 | 25.76 |
| ATOM | 4384 | CB | THR | B | 232 | 42.460 | 103.926 | 17.200 | 1.00 | 26.18 |
| ATOM | 4385 | OG1 | THR | B | 232 | 43.518 | 103.434 | 16.410 | 1.00 | 32.28 |
| ATOM | 4386 | CG2 | THR | B | 232 | 43.039 | 104.388 | 18.520 | 1.00 | 27.67 |
| ATOM | 4387 | C | THR | B | 232 | 41.889 | 101.622 | 18.015 | 1.00 | 26.18 |
| ATOM | 4388 | O | THR | B | 232 | 42.769 | 100.948 | 17.476 | 1.00 | 25.89 |
| ATOM | 4389 | N | ASP | B | 233 | 41.361 | 101.306 | 19.206 | 1.00 | 25.99 |
| ATOM | 4390 | CA | ASP | B | 233 | 41.795 | 100.134 | 19.967 | 1.00 | 24.56 |
| ATOM | 4391 | CB | ASP | B | 233 | 42.508 | 100.609 | 21.257 | 1.00 | 28.62 |
| ATOM | 4392 | CG | ASP | B | 233 | 42.360 | 99.698 | 22.462 | 1.00 | 30.06 |
| ATOM | 4393 | OD1 | ASP | B | 233 | 41.434 | 99.937 | 23.262 | 1.00 | 34.24 |
| ATOM | 4394 | OD2 | ASP | B | 233 | 43.170 | 98.751 | 22.607 | 1.00 | 31.50 |
| ATOM | 4395 | C | ASP | B | 233 | 40.642 | 99.138 | 20.217 | 1.00 | 23.91 |
| ATOM | 4396 | O | ASP | B | 233 | 40.721 | 98.264 | 21.080 | 1.00 | 26.44 |
| ATOM | 4397 | N | TYR | B | 234 | 39.582 | 99.258 | 19.426 | 1.00 | 22.91 |
| ATOM | 4398 | CA | TYR | B | 234 | 38.416 | 98.374 | 19.516 | 1.00 | 20.38 |
| ATOM | 4399 | CB | TYR | B | 234 | 37.380 | 98.748 | 18.452 | 1.00 | 23.38 |
| ATOM | 4400 | CG | TYR | B | 234 | 37.960 | 98.779 | 17.059 | 1.00 | 22.55 |
| ATOM | 4401 | CD1 | TYR | B | 234 | 38.028 | 97.621 | 16.292 | 1.00 | 24.15 |
| ATOM | 4402 | CE1 | TYR | B | 234 | 38.586 | 97.634 | 15.026 | 1.00 | 24.83 |
| ATOM | 4403 | CD2 | TYR | B | 234 | 38.463 | 99.958 | 16.524 | 1.00 | 24.39 |
| ATOM | 4404 | CE2 | TYR | B | 234 | 39.020 | 99.987 | 15.256 | 1.00 | 25.51 |
| ATOM | 4405 | CZ | TYR | B | 234 | 39.079 | 98.816 | 14.513 | 1.00 | 26.35 |
| ATOM | 4406 | OH | TYR | B | 234 | 39.637 | 98.829 | 13.264 | 1.00 | 28.21 |
| ATOM | 4407 | C | TYR | B | 234 | 38.799 | 96.894 | 19.394 | 1.00 | 16.79 |
| ATOM | 4408 | O | TYR | B | 234 | 39.955 | 96.553 | 19.144 | 1.00 | 19.38 |
| ATOM | 4409 | N | THR | B | 235 | 37.811 | 96.028 | 19.600 | 1.00 | 13.77 |
| ATOM | 4410 | CA | THR | B | 235 | 37.994 | 94.578 | 19.559 | 1.00 | 15.58 |
| ATOM | 4411 | CB | THR | B | 235 | 37.720 | 93.997 | 20.961 | 1.00 | 11.90 |
| ATOM | 4412 | OG1 | THR | B | 235 | 36.323 | 93.922 | 21.190 | 1.00 | 11.51 |
| ATOM | 4413 | CG2 | THR | B | 235 | 38.331 | 94.801 | 22.094 | 1.00 | 14.31 |
| ATOM | 4414 | C | THR | B | 235 | 37.020 | 93.927 | 18.573 | 1.00 | 14.16 |
| ATOM | 4415 | O | THR | B | 235 | 36.270 | 94.612 | 17.896 | 1.00 | 16.98 |
| ATOM | 4416 | N | SER | B | 236 | 37.034 | 92.597 | 18.544 | 1.00 | 17.59 |
| ATOM | 4417 | CA | SER | B | 236 | 36.164 | 91.783 | 17.682 | 1.00 | 18.69 |
| ATOM | 4418 | CB | SER | B | 236 | 36.558 | 90.315 | 17.749 | 1.00 | 18.45 |
| ATOM | 4419 | OG | SER | B | 236 | 37.774 | 90.092 | 17.041 | 1.00 | 24.22 |
| ATOM | 4420 | C | SER | B | 236 | 34.681 | 91.961 | 17.979 | 1.00 | 20.02 |
| ATOM | 4421 | O | SER | B | 236 | 33.818 | 91.472 | 17.225 | 1.00 | 20.26 |
| ATOM | 4422 | N | SER | B | 237 | 34.377 | 92.686 | 19.053 | 1.00 | 18.28 |
| ATOM | 4423 | CA | SER | B | 237 | 32.998 | 92.964 | 19.428 | 1.00 | 14.71 |
| ATOM | 4424 | CB | SER | B | 237 | 32.950 | 93.669 | 20.791 | 1.00 | 15.53 |
| ATOM | 4425 | OG | SER | B | 237 | 33.890 | 94.728 | 20.825 | 1.00 | 16.04 |
| ATOM | 4426 | C | SER | B | 237 | 32.313 | 93.853 | 18.388 | 1.00 | 12.17 |
| ATOM | 4427 | O | SER | B | 237 | 31.092 | 93.914 | 18.335 | 1.00 | 8.50 |
| ATOM | 4428 | N | ILE | B | 238 | 33.086 | 94.551 | 17.552 | 1.00 | 14.47 |
| ATOM | 4429 | CA | ILE | B | 238 | 32.436 | 95.390 | 16.518 | 1.00 | 16.29 |
| ATOM | 4430 | CB | ILE | B | 238 | 33.426 | 96.260 | 15.714 | 1.00 | 16.74 |
| ATOM | 4431 | CG2 | ILE | B | 238 | 34.121 | 97.275 | 16.622 | 1.00 | 16.15 |
| ATOM | 4432 | CG1 | ILE | B | 238 | 34.444 | 95.401 | 14.951 | 1.00 | 13.48 |
| ATOM | 4433 | CD1 | ILE | B | 238 | 35.264 | 96.191 | 13.953 | 1.00 | 13.12 |
| ATOM | 4434 | C | ILE | B | 238 | 31.608 | 94.500 | 15.573 | 1.00 | 14.21 |
| ATOM | 4435 | O | ILE | B | 238 | 30.516 | 94.872 | 15.157 | 1.00 | 18.18 |
| ATOM | 4436 | N | ASP | B | 239 | 32.119 | 93.296 | 15.293 | 1.00 | 15.25 |
| ATOM | 4437 | CA | ASP | B | 239 | 31.423 | 92.306 | 14.442 | 1.00 | 15.37 |
| ATOM | 4438 | CB | ASP | B | 239 | 32.281 | 91.036 | 14.263 | 1.00 | 13.37 |
| ATOM | 4439 | CG | ASP | B | 239 | 33.405 | 91.194 | 13.273 | 1.00 | 13.63 |
| ATOM | 4440 | OD1 | ASP | B | 239 | 33.409 | 92.200 | 12.542 | 1.00 | 14.70 |
| ATOM | 4441 | OD2 | ASP | B | 239 | 34.298 | 90.306 | 13.236 | 1.00 | 16.99 |
| ATOM | 4442 | C | ASP | B | 239 | 30.089 | 91.908 | 15.064 | 1.00 | 16.77 |
| ATOM | 4443 | O | ASP | B | 239 | 29.076 | 91.742 | 14.355 | 1.00 | 16.53 |
| ATOM | 4444 | N | VAL | B | 240 | 30.072 | 91.754 | 16.416 | 1.00 | 15.89 |
| ATOM | 4445 | CA | VAL | B | 240 | 28.829 | 91.378 | 17.109 | 1.00 | 12.01 |
| ATOM | 4446 | CB | VAL | B | 240 | 29.046 | 91.059 | 18.624 | 1.00 | 14.35 |
| ATOM | 4447 | CG1 | VAL | B | 240 | 27.737 | 91.199 | 19.387 | 1.00 | 9.00 |
| ATOM | 4448 | CG2 | VAL | B | 240 | 29.604 | 89.650 | 18.790 | 1.00 | 13.00 |
| ATOM | 4449 | C | VAL | B | 240 | 27.787 | 92.464 | 16.952 | 1.00 | 8.73 |
| ATOM | 4450 | O | VAL | B | 240 | 26.603 | 92.192 | 16.799 | 1.00 | 14.10 |
| ATOM | 4451 | N | TRP | B | 241 | 28.227 | 93.698 | 16.967 | 1.00 | 10.49 |
| ATOM | 4452 | CA | TRP | B | 241 | 27.303 | 94.807 | 16.790 | 1.00 | 13.99 |
| ATOM | 4453 | CB | TRP | B | 241 | 28.004 | 96.139 | 17.103 | 1.00 | 11.75 |
| ATOM | 4454 | CG | TRP | B | 241 | 27.184 | 97.333 | 16.716 | 1.00 | 12.61 |
| ATOM | 4455 | CD2 | TRP | B | 241 | 26.341 | 98.132 | 17.564 | 1.00 | 18.21 |
| ATOM | 4456 | CE2 | TRP | B | 241 | 25.748 | 99.124 | 16.747 | 1.00 | 18.68 |
| ATOM | 4457 | CE3 | TRP | B | 241 | 26.027 | 98.109 | 18.931 | 1.00 | 18.35 |
| ATOM | 4458 | CD1 | TRP | B | 241 | 27.074 | 97.867 | 15.465 | 1.00 | 15.24 |
| ATOM | 4459 | NE1 | TRP | B | 241 | 26.205 | 98.929 | 15.473 | 1.00 | 18.13 |
| ATOM | 4460 | CZ2 | TRP | B | 241 | 24.861 | 100.078 | 17.245 | 1.00 | 21.75 |
| ATOM | 4461 | CZ3 | TRP | B | 241 | 25.150 | 99.051 | 19.426 | 1.00 | 21.43 |
| ATOM | 4462 | CH2 | TRP | B | 241 | 24.572 | 100.024 | 18.588 | 1.00 | 24.55 |
| ATOM | 4463 | C | TRP | B | 241 | 26.728 | 94.791 | 15.340 | 1.00 | 12.69 |
| ATOM | 4464 | O | TRP | B | 241 | 25.543 | 95.009 | 15.131 | 1.00 | 12.66 |
| ATOM | 4465 | N | SER | B | 242 | 27.588 | 94.505 | 14.349 | 1.00 | 14.14 |
| ATOM | 4466 | CA | SER | B | 242 | 27.148 | 94.435 | 12.938 | 1.00 | 14.63 |
| ATOM | 4467 | CB | SER | B | 242 | 28.327 | 94.072 | 12.029 | 1.00 | 10.79 |
| ATOM | 4468 | OG | SER | B | 242 | 29.311 | 95.084 | 12.076 | 1.00 | 12.39 |
| ATOM | 4469 | C | SER | B | 242 | 26.071 | 93.386 | 12.810 | 1.00 | 13.69 |
| ATOM | 4470 | O | SER | B | 242 | 24.987 | 93.650 | 12.297 | 1.00 | 15.83 |
| ATOM | 4471 | N | ALA | B | 243 | 26.370 | 92.194 | 13.328 | 1.00 | 15.65 |
| ATOM | 4472 | CA | ALA | B | 243 | 25.426 | 91.088 | 13.316 | 1.00 | 16.55 |
| ATOM | 4473 | CB | ALA | B | 243 | 26.034 | 89.868 | 13.981 | 1.00 | 17.84 |
| ATOM | 4474 | C | ALA | B | 243 | 24.106 | 91.483 | 13.974 | 1.00 | 17.41 |
| ATOM | 4475 | O | ALA | B | 243 | 23.037 | 90.993 | 13.589 | 1.00 | 17.38 |
| ATOM | 4476 | N | GLY | B | 244 | 24.175 | 92.403 | 14.951 | 1.00 | 19.03 |
| ATOM | 4477 | CA | GLY | B | 244 | 22.957 | 92.867 | 15.607 | 1.00 | 17.62 |
| ATOM | 4478 | C | GLY | B | 244 | 22.158 | 93.748 | 14.677 | 1.00 | 17.92 |
| ATOM | 4479 | O | GLY | B | 244 | 20.942 | 93.632 | 14.577 | 1.00 | 17.59 |
| ATOM | 4480 | N | CYS | B | 245 | 22.854 | 94.609 | 13.952 | 1.00 | 19.90 |
| ATOM | 4481 | CA | CYS | B | 245 | 22.191 | 95.467 | 12.974 | 1.00 | 19.65 |
| ATOM | 4482 | CB | CYS | B | 245 | 23.201 | 96.399 | 12.318 | 1.00 | 19.83 |
| ATOM | 4483 | SG | CYS | B | 245 | 23.913 | 97.637 | 13.424 | 1.00 | 22.50 |
| ATOM | 4484 | C | CYS | B | 245 | 21.537 | 94.590 | 11.906 | 1.00 | 18.91 |
| ATOM | 4485 | O | CYS | B | 245 | 20.475 | 94.911 | 11.394 | 1.00 | 22.00 |
| ATOM | 4486 | N | VAL | B | 246 | 22.178 | 93.463 | 11.596 | 1.00 | 20.35 |
| ATOM | 4487 | CA | VAL | B | 246 | 21.664 | 92.510 | 10.606 | 1.00 | 20.75 |
| ATOM | 4488 | CB | VAL | B | 246 | 22.680 | 91.385 | 10.324 | 1.00 | 18.97 |
| ATOM | 4489 | CG1 | VAL | B | 246 | 22.076 | 90.328 | 9.398 | 1.00 | 19.70 |
| ATOM | 4490 | CG2 | VAL | B | 246 | 23.965 | 91.952 | 9.752 | 1.00 | 19.92 |
| ATOM | 4491 | C | VAL | B | 246 | 20.374 | 91.877 | 11.094 | 1.00 | 22.64 |
| ATOM | 4492 | O | VAL | B | 246 | 19.347 | 91.905 | 10.400 | 1.00 | 26.23 |
| ATOM | 4493 | N | LEU | B | 247 | 20.425 | 91.310 | 12.308 | 1.00 | 22.64 |
| ATOM | 4494 | CA | LEU | B | 247 | 19.262 | 90.672 | 12.914 | 1.00 | 19.95 |
| ATOM | 4495 | CB | LEU | B | 247 | 19.630 | 90.135 | 14.316 | 1.00 | 22.43 |
| ATOM | 4496 | CG | LEU | B | 247 | 18.486 | 89.838 | 15.293 | 1.00 | 20.19 |
| ATOM | 4497 | CD1 | LEU | B | 247 | 17.871 | 91.125 | 15.811 | 1.00 | 22.92 |
| ATOM | 4498 | CD2 | LEU | B | 247 | 17.428 | 88.951 | 14.659 | 1.00 | 21.05 |
| ATOM | 4499 | C | LEU | B | 247 | 18.082 | 91.632 | 12.975 | 1.00 | 20.28 |
| ATOM | 4500 | O | LEU | B | 247 | 16.959 | 91.267 | 12.620 | 1.00 | 21.69 |
| ATOM | 4501 | N | ALA | B | 248 | 18.337 | 92.864 | 13.432 | 1.00 | 20.15 |
| ATOM | 4502 | CA | ALA | B | 248 | 17.281 | 93.873 | 13.543 | 1.00 | 20.23 |
| ATOM | 4503 | CB | ALA | B | 248 | 17.795 | 95.114 | 14.261 | 1.00 | 17.38 |
| ATOM | 4504 | C | ALA | B | 248 | 16.718 | 94.230 | 12.162 | 1.00 | 19.28 |
| ATOM | 4505 | O | ALA | B | 248 | 15.507 | 94.393 | 11.995 | 1.00 | 17.25 |
| ATOM | 4506 | N | GLU | B | 249 | 17.606 | 94.322 | 11.171 | 1.00 | 21.23 |
| ATOM | 4507 | CA | GLU | B | 249 | 17.186 | 94.620 | 9.798 | 1.00 | 20.11 |
| ATOM | 4508 | CB | GLU | B | 249 | 18.404 | 94.766 | 8.867 | 1.00 | 22.01 |
| ATOM | 4509 | CG | GLU | B | 249 | 18.081 | 95.498 | 7.573 | 1.00 | 24.30 |
| ATOM | 4510 | CD | GLU | B | 249 | 19.266 | 95.673 | 6.650 | 1.00 | 24.80 |
| ATOM | 4511 | OE1 | GLU | B | 249 | 20.400 | 95.818 | 7.144 | 1.00 | 25.98 |
| ATOM | 4512 | OE2 | GLU | B | 249 | 19.054 | 95.684 | 5.420 | 1.00 | 27.86 |
| ATOM | 4513 | C | GLU | B | 249 | 16.229 | 93.521 | 9.301 | 1.00 | 17.75 |
| ATOM | 4514 | O | GLU | B | 249 | 15.170 | 93.808 | 8.766 | 1.00 | 18.76 |
| ATOM | 4515 | N | LEU | B | 250 | 16.594 | 92.262 | 9.536 | 1.00 | 19.74 |
| ATOM | 4516 | CA | LEU | B | 250 | 15.762 | 91.119 | 9.146 | 1.00 | 21.76 |
| ATOM | 4517 | CB | LEU | B | 250 | 16.456 | 89.814 | 9.498 | 1.00 | 21.71 |
| ATOM | 4518 | CG | LEU | B | 250 | 17.846 | 89.609 | 8.911 | 1.00 | 24.86 |
| ATOM | 4519 | CD1 | LEU | B | 250 | 18.390 | 88.246 | 9.311 | 1.00 | 22.96 |
| ATOM | 4520 | CD2 | LEU | B | 250 | 17.816 | 89.758 | 7.392 | 1.00 | 23.16 |
| ATOM | 4521 | C | LEU | B | 250 | 14.372 | 91.136 | 9.783 | 1.00 | 23.01 |
| ATOM | 4522 | O | LEU | B | 250 | 13.395 | 90.722 | 9.156 | 1.00 | 22.18 |
| ATOM | 4523 | N | LEU | B | 251 | 14.287 | 91.592 | 11.039 | 1.00 | 23.52 |
| ATOM | 4524 | CA | LEU | B | 251 | 13.016 | 91.639 | 11.765 | 1.00 | 23.75 |
| ATOM | 4525 | CB | LEU | B | 251 | 13.267 | 91.681 | 13.284 | 1.00 | 22.85 |
| ATOM | 4526 | CG | LEU | B | 251 | 14.091 | 90.544 | 13.881 | 1.00 | 23.41 |
| ATOM | 4527 | CD1 | LEU | B | 251 | 14.665 | 90.959 | 15.226 | 1.00 | 25.36 |
| ATOM | 4528 | CD2 | LEU | B | 251 | 13.244 | 89.291 | 14.029 | 1.00 | 23.22 |
| ATOM | 4529 | C | LEU | B | 251 | 12.201 | 92.866 | 11.384 | 1.00 | 25.00 |
| ATOM | 4530 | O | LEU | B | 251 | 10.976 | 92.808 | 11.306 | 1.00 | 25.53 |
| ATOM | 4531 | N | LEU | B | 252 | 12.895 | 93.982 | 11.191 | 1.00 | 27.31 |
| ATOM | 4532 | CA | LEU | B | 252 | 12.275 | 95.270 | 10.862 | 1.00 | 28.85 |
| ATOM | 4533 | CB | LEU | B | 252 | 13.247 | 96.394 | 11.208 | 1.00 | 29.10 |
| ATOM | 4534 | CG | LEU | B | 252 | 12.820 | 97.347 | 12.316 | 1.00 | 30.29 |
| ATOM | 4535 | CD1 | LEU | B | 252 | 13.857 | 98.451 | 12.478 | 1.00 | 30.64 |
| ATOM | 4536 | CD2 | LEU | B | 252 | 11.453 | 97.937 | 12.008 | 1.00 | 29.71 |
| ATOM | 4537 | C | LEU | B | 252 | 11.860 | 95.417 | 9.396 | 1.00 | 29.11 |
| ATOM | 4538 | O | LEU | B | 252 | 11.009 | 96.245 | 9.077 | 1.00 | 29.16 |
| ATOM | 4539 | N | GLY | B | 253 | 12.483 | 94.655 | 8.506 | 1.00 | 30.27 |
| ATOM | 4540 | CA | GLY | B | 253 | 12.154 | 94.786 | 7.092 | 1.00 | 30.40 |
| ATOM | 4541 | C | GLY | B | 253 | 12.909 | 95.940 | 6.447 | 1.00 | 31.85 |
| ATOM | 4542 | O | GLY | B | 253 | 12.677 | 96.272 | 5.291 | 1.00 | 31.62 |
| ATOM | 4543 | N | GLN | B | 254 | 13.825 | 96.545 | 7.212 | 1.00 | 32.10 |
| ATOM | 4544 | CA | GLN | B | 254 | 14.653 | 97.665 | 6.754 | 1.00 | 30.81 |
| ATOM | 4545 | CB | GLN | B | 254 | 13.814 | 98.933 | 6.559 | 1.00 | 31.22 |
| ATOM | 4546 | CG | GLN | B | 254 | 13.195 | 99.477 | 7.836 | 1.00 | 31.70 |
| ATOM | 4547 | CD | GLN | B | 254 | 12.307 | 100.677 | 7.595 | 1.00 | 30.46 |
| ATOM | 4548 | OE1 | GLN | B | 254 | 12.766 | 101.814 | 7.605 | 1.00 | 33.00 |
| ATOM | 4549 | NE2 | GLN | B | 254 | 11.023 | 100.432 | 7.382 | 1.00 | 32.32 |
| ATOM | 4550 | C | GLN | B | 254 | 15.777 | 97.915 | 7.759 | 1.00 | 30.56 |
| ATOM | 4551 | O | GLN | B | 254 | 15.697 | 97.455 | 8.905 | 1.00 | 30.99 |
| ATOM | 4552 | N | PRO | B | 255 | 16.849 | 98.631 | 7.351 | 1.00 | 28.81 |
| ATOM | 4553 | CD | PRO | B | 255 | 17.047 | 99.200 | 6.003 | 1.00 | 29.65 |
| ATOM | 4554 | CA | PRO | B | 255 | 17.990 | 98.918 | 8.228 | 1.00 | 26.75 |
| ATOM | 4555 | CB | PRO | B | 255 | 18.932 | 99.751 | 7.346 | 1.00 | 28.90 |
| ATOM | 4556 | CG | PRO | B | 255 | 18.535 | 99.399 | 5.948 | 1.00 | 28.37 |
| ATOM | 4557 | C | PRO | B | 255 | 17.565 | 99.701 | 9.461 | 1.00 | 25.44 |
| ATOM | 4558 | O | PRO | B | 255 | 16.855 | 100.696 | 9.371 | 1.00 | 23.84 |
| ATOM | 4559 | N | ILE | B | 256 | 17.998 | 99.221 | 10.617 | 1.00 | 25.79 |
| ATOM | 4560 | CA | ILE | B | 256 | 17.662 | 99.842 | 11.893 | 1.00 | 22.03 |
| ATOM | 4561 | CB | ILE | B | 256 | 18.048 | 98.931 | 13.089 | 1.00 | 20.49 |
| ATOM | 4562 | CG2 | ILE | B | 256 | 19.519 | 98.531 | 13.046 | 1.00 | 16.83 |
| ATOM | 4563 | CG1 | ILE | B | 256 | 17.676 | 99.562 | 14.444 | 1.00 | 22.30 |
| ATOM | 4564 | CD1 | ILE | B | 256 | 16.380 | 100.346 | 14.446 | 1.00 | 22.57 |
| ATOM | 4565 | C | ILE | B | 256 | 18.218 | 101.261 | 12.029 | 1.00 | 20.33 |
| ATOM | 4566 | O | ILE | B | 256 | 17.506 | 102.163 | 12.473 | 1.00 | 23.40 |
| ATOM | 4567 | N | PHE | B | 257 | 19.472 | 101.468 | 11.641 | 1.00 | 20.97 |
| ATOM | 4568 | CA | PHE | B | 257 | 20.091 | 102.794 | 11.734 | 1.00 | 21.60 |
| ATOM | 4569 | CB | PHE | B | 257 | 21.223 | 102.800 | 12.777 | 1.00 | 21.88 |
| ATOM | 4570 | CG | PHE | B | 257 | 20.968 | 101.972 | 14.019 | 1.00 | 20.01 |
| ATOM | 4571 | CD1 | PHE | B | 257 | 19.896 | 102.256 | 14.857 | 1.00 | 20.95 |
| ATOM | 4572 | CD2 | PHE | B | 257 | 21.811 | 100.924 | 14.350 | 1.00 | 19.46 |
| ATOM | 4573 | CE1 | PHE | B | 257 | 19.677 | 101.507 | 16.001 | 1.00 | 21.15 |
| ATOM | 4574 | CE2 | PHE | B | 257 | 21.595 | 100.170 | 15.494 | 1.00 | 20.25 |
| ATOM | 4575 | CZ | PHE | B | 257 | 20.527 | 100.461 | 16.319 | 1.00 | 17.61 |
| ATOM | 4576 | C | PHE | B | 257 | 20.625 | 103.303 | 10.378 | 1.00 | 23.72 |
| ATOM | 4577 | O | PHE | B | 257 | 21.799 | 103.116 | 10.050 | 1.00 | 24.92 |
| ATOM | 4578 | N | PRO | B | 258 | 19.774 | 103.959 | 9.571 | 1.00 | 24.77 |
| ATOM | 4579 | CD | PRO | B | 258 | 18.355 | 104.232 | 9.859 | 1.00 | 24.97 |
| ATOM | 4580 | CA | PRO | B | 258 | 20.173 | 104.492 | 8.258 | 1.00 | 26.83 |
| ATOM | 4581 | CB | PRO | B | 258 | 18.838 | 104.562 | 7.526 | 1.00 | 25.37 |
| ATOM | 4582 | CG | PRO | B | 258 | 17.861 | 104.907 | 8.601 | 1.00 | 26.59 |
| ATOM | 4583 | C | PRO | B | 258 | 20.801 | 105.892 | 8.333 | 1.00 | 27.49 |
| ATOM | 4584 | O | PRO | B | 258 | 20.148 | 106.890 | 8.027 | 1.00 | 29.44 |
| ATOM | 4585 | N | GLY | B | 259 | 22.072 | 105.958 | 8.723 | 1.00 | 28.58 |
| ATOM | 4586 | CA | GLY | B | 259 | 22.763 | 107.238 | 8.809 | 1.00 | 30.88 |
| ATOM | 4587 | C | GLY | B | 259 | 23.066 | 107.815 | 7.430 | 1.00 | 32.75 |
| ATOM | 4588 | O | GLY | B | 259 | 23.074 | 107.080 | 6.441 | 1.00 | 32.73 |
| ATOM | 4589 | N | ASP | B | 260 | 23.304 | 109.127 | 7.353 | 1.00 | 33.18 |
| ATOM | 4590 | CA | ASP | B | 260 | 23.580 | 109.762 | 6.066 | 1.00 | 34.42 |
| ATOM | 4591 | CB | ASP | B | 260 | 22.530 | 110.847 | 5.730 | 1.00 | 35.56 |
| ATOM | 4592 | CG | ASP | B | 260 | 22.547 | 112.072 | 6.649 | 1.00 | 37.21 |
| ATOM | 4593 | OD1 | ASP | B | 260 | 21.477 | 112.692 | 6.836 | 1.00 | 38.35 |
| ATOM | 4594 | OD2 | ASP | B | 260 | 23.622 | 112.424 | 7.172 | 1.00 | 41.05 |
| ATOM | 4595 | C | ASP | B | 260 | 25.004 | 110.298 | 5.897 | 1.00 | 34.80 |
| ATOM | 4596 | O | ASP | B | 260 | 25.282 | 110.976 | 4.901 | 1.00 | 35.69 |
| ATOM | 4597 | N | SER | B | 261 | 25.919 | 110.032 | 6.843 | 1.00 | 34.20 |
| ATOM | 4598 | CA | SER | B | 261 | 27.262 | 110.591 | 6.653 | 1.00 | 33.70 |
| ATOM | 4599 | CB | SER | B | 261 | 27.282 | 112.065 | 7.073 | 1.00 | 34.65 |
| ATOM | 4600 | OG | SER | B | 261 | 26.660 | 112.247 | 8.333 | 1.00 | 34.15 |
| ATOM | 4601 | C | SER | B | 261 | 28.405 | 109.854 | 7.338 | 1.00 | 33.14 |
| ATOM | 4602 | O | SER | B | 261 | 29.513 | 109.776 | 6.792 | 1.00 | 35.18 |
| ATOM | 4603 | N | GLY | B | 262 | 28.169 | 109.389 | 8.546 | 1.00 | 31.51 |
| ATOM | 4604 | CA | GLY | B | 262 | 29.215 | 108.741 | 9.316 | 1.00 | 28.23 |
| ATOM | 4605 | C | GLY | B | 262 | 29.127 | 109.228 | 10.749 | 1.00 | 29.20 |
| ATOM | 4606 | O | GLY | B | 262 | 29.340 | 108.475 | 11.699 | 1.00 | 27.66 |
| ATOM | 4607 | N | VAL | B | 263 | 28.734 | 110.491 | 10.879 | 1.00 | 28.32 |
| ATOM | 4608 | CA | VAL | B | 263 | 28.510 | 111.127 | 12.161 | 1.00 | 27.43 |
| ATOM | 4609 | CB | VAL | B | 263 | 28.983 | 112.591 | 12.157 | 1.00 | 27.60 |
| ATOM | 4610 | CG1 | VAL | B | 263 | 28.678 | 113.246 | 13.498 | 1.00 | 28.91 |
| ATOM | 4611 | CG2 | VAL | B | 263 | 30.474 | 112.661 | 11.844 | 1.00 | 26.99 |
| ATOM | 4612 | C | VAL | B | 263 | 27.012 | 111.072 | 12.434 | 1.00 | 27.34 |
| ATOM | 4613 | O | VAL | B | 263 | 26.580 | 110.866 | 13.567 | 1.00 | 27.77 |
| ATOM | 4614 | N | ASP | B | 264 | 26.226 | 111.198 | 11.351 | 1.00 | 24.33 |
| ATOM | 4615 | CA | ASP | B | 264 | 24.772 | 111.100 | 11.420 | 1.00 | 22.03 |
| ATOM | 4616 | CB | ASP | B | 264 | 24.145 | 111.447 | 10.064 | 1.00 | 22.91 |
| ATOM | 4617 | CG | ASP | B | 264 | 22.630 | 111.375 | 10.086 | 1.00 | 23.28 |
| ATOM | 4618 | OD1 | ASP | B | 264 | 22.010 | 112.133 | 10.859 | 1.00 | 27.20 |
| ATOM | 4619 | OD2 | ASP | B | 264 | 22.062 | 110.557 | 9.339 | 1.00 | 28.80 |
| ATOM | 4620 | C | ASP | B | 264 | 24.384 | 109.673 | 11.814 | 1.00 | 20.79 |
| ATOM | 4621 | O | ASP | B | 264 | 23.292 | 109.420 | 12.307 | 1.00 | 22.24 |
| ATOM | 4622 | N | GLN | B | 265 | 25.305 | 108.749 | 11.607 | 1.00 | 19.00 |
| ATOM | 4623 | CA | GLN | B | 265 | 25.086 | 107.361 | 11.963 | 1.00 | 20.26 |
| ATOM | 4624 | CB | GLN | B | 265 | 26.275 | 106.513 | 11.496 | 1.00 | 16.83 |
| ATOM | 4625 | CG | GLN | B | 265 | 26.099 | 105.018 | 11.692 | 1.00 | 22.64 |
| ATOM | 4626 | CD | GLN | B | 265 | 24.868 | 104.484 | 10.994 | 1.00 | 24.93 |
| ATOM | 4627 | OE1 | GLN | B | 265 | 24.425 | 105.033 | 9.974 | 1.00 | 27.28 |
| ATOM | 4628 | NE2 | GLN | B | 265 | 24.308 | 103.408 | 11.538 | 1.00 | 26.87 |
| ATOM | 4629 | C | GLN | B | 265 | 24.892 | 107.241 | 13.493 | 1.00 | 20.32 |
| ATOM | 4630 | O | GLN | B | 265 | 24.091 | 106.433 | 13.951 | 1.00 | 19.37 |
| ATOM | 4631 | N | LEU | B | 266 | 25.612 | 108.079 | 14.275 | 1.00 | 22.23 |
| ATOM | 4632 | CA | LEU | B | 266 | 25.469 | 108.062 | 15.742 | 1.00 | 24.94 |
| ATOM | 4633 | CB | LEU | B | 266 | 26.575 | 108.844 | 16.472 | 1.00 | 22.54 |
| ATOM | 4634 | CG | LEU | B | 266 | 26.592 | 108.702 | 18.020 | 1.00 | 22.64 |
| ATOM | 4635 | CD1 | LEU | B | 266 | 26.751 | 107.248 | 18.440 | 1.00 | 18.03 |
| ATOM | 4636 | CD2 | LEU | B | 266 | 27.695 | 109.542 | 18.637 | 1.00 | 19.32 |
| ATOM | 4637 | C | LEU | B | 266 | 24.099 | 108.589 | 16.134 | 1.00 | 25.37 |
| ATOM | 4638 | O | LEU | B | 266 | 23.478 | 108.074 | 17.064 | 1.00 | 28.29 |
| ATOM | 4639 | N | VAL | B | 267 | 23.625 | 109.599 | 15.391 | 1.00 | 24.88 |
| ATOM | 4640 | CA | VAL | B | 267 | 22.312 | 110.198 | 15.622 | 1.00 | 24.12 |
| ATOM | 4641 | CB | VAL | B | 267 | 22.065 | 111.430 | 14.716 | 1.00 | 24.95 |
| ATOM | 4642 | CG1 | VAL | B | 267 | 20.651 | 111.967 | 14.901 | 1.00 | 23.98 |
| ATOM | 4643 | CG2 | VAL | B | 267 | 23.083 | 112.522 | 15.004 | 1.00 | 25.79 |
| ATOM | 4644 | C | VAL | B | 267 | 21.183 | 109.182 | 15.424 | 1.00 | 24.25 |
| ATOM | 4645 | O | VAL | B | 267 | 20.201 | 109.176 | 16.177 | 1.00 | 23.82 |
| ATOM | 4646 | N | GLU | B | 268 | 21.319 | 108.324 | 14.408 | 1.00 | 25.05 |
| ATOM | 4647 | CA | GLU | B | 268 | 20.299 | 107.307 | 14.122 | 1.00 | 23.51 |
| ATOM | 4648 | CB | GLU | B | 268 | 20.515 | 106.693 | 12.732 | 1.00 | 24.47 |
| ATOM | 4649 | CG | GLU | B | 268 | 20.293 | 107.676 | 11.584 | 1.00 | 21.75 |
| ATOM | 4650 | CD | GLU | B | 268 | 18.934 | 108.349 | 11.638 | 1.00 | 18.57 |
| ATOM | 4651 | OE1 | GLU | B | 268 | 18.881 | 109.594 | 11.611 | 1.00 | 22.02 |
| ATOM | 4652 | OE2 | GLU | B | 268 | 17.923 | 107.632 | 11.706 | 1.00 | 23.39 |
| ATOM | 4653 | C | GLU | B | 268 | 20.292 | 106.224 | 15.207 | 1.00 | 23.03 |
| ATOM | 4654 | O | GLU | B | 268 | 19.237 | 105.731 | 15.613 | 1.00 | 22.81 |
| ATOM | 4655 | N | ILE | B | 269 | 21.472 | 105.884 | 15.689 | 1.00 | 21.74 |
| ATOM | 4656 | CA | ILE | B | 269 | 21.612 | 104.898 | 16.744 | 1.00 | 24.46 |
| ATOM | 4657 | CB | ILE | B | 269 | 23.102 | 104.574 | 16.970 | 1.00 | 23.40 |
| ATOM | 4658 | CG2 | ILE | B | 269 | 23.314 | 103.814 | 18.275 | 1.00 | 22.87 |
| ATOM | 4659 | CG1 | ILE | B | 269 | 23.656 | 103.780 | 15.776 | 1.00 | 20.00 |
| ATOM | 4660 | CD1 | ILE | B | 269 | 25.164 | 103.648 | 15.773 | 1.00 | 14.35 |
| ATOM | 4661 | C | ILE | B | 269 | 20.998 | 105.450 | 18.041 | 1.00 | 26.26 |
| ATOM | 4662 | O | ILE | B | 269 | 20.166 | 104.797 | 18.672 | 1.00 | 26.78 |
| ATOM | 4663 | N | ILE | B | 270 | 21.406 | 106.677 | 18.404 | 1.00 | 27.54 |
| ATOM | 4664 | CA | ILE | B | 270 | 20.911 | 107.364 | 19.600 | 1.00 | 29.63 |
| ATOM | 4665 | CB | ILE | B | 270 | 21.524 | 108.780 | 19.734 | 1.00 | 27.82 |
| ATOM | 4666 | CG2 | ILE | B | 270 | 20.561 | 109.740 | 20.414 | 1.00 | 27.92 |
| ATOM | 4667 | CG1 | ILE | B | 270 | 22.859 | 108.714 | 20.483 | 1.00 | 24.59 |
| ATOM | 4668 | CD1 | ILE | B | 270 | 23.784 | 109.873 | 20.192 | 1.00 | 20.57 |
| ATOM | 4669 | C | ILE | B | 270 | 19.380 | 107.439 | 19.625 | 1.00 | 31.35 |
| ATOM | 4670 | O | ILE | B | 270 | 18.766 | 107.336 | 20.688 | 1.00 | 31.68 |
| ATOM | 4671 | N | LYS | B | 271 | 18.762 | 107.605 | 18.456 | 1.00 | 31.75 |
| ATOM | 4672 | CA | LYS | B | 271 | 17.298 | 107.675 | 18.368 | 1.00 | 31.08 |
| ATOM | 4673 | CB | LYS | B | 271 | 16.859 | 107.825 | 16.907 | 1.00 | 32.98 |
| ATOM | 4674 | CG | LYS | B | 271 | 16.767 | 109.266 | 16.432 | 1.00 | 35.15 |
| ATOM | 4675 | CD | LYS | B | 271 | 15.829 | 109.383 | 15.237 | 1.00 | 39.78 |
| ATOM | 4676 | CE | LYS | B | 271 | 16.337 | 110.374 | 14.199 | 1.00 | 40.13 |
| ATOM | 4677 | NZ | LYS | B | 271 | 15.626 | 110.220 | 12.895 | 1.00 | 43.28 |
| ATOM | 4678 | C | LYS | B | 271 | 16.635 | 106.430 | 18.973 | 1.00 | 31.09 |
| ATOM | 4679 | O | LYS | B | 271 | 15.500 | 106.495 | 19.450 | 1.00 | 31.32 |
| ATOM | 4680 | N | VAL | B | 272 | 17.341 | 105.297 | 18.936 | 1.00 | 30.13 |
| ATOM | 4681 | CA | VAL | B | 272 | 16.820 | 104.033 | 19.463 | 1.00 | 29.49 |
| ATOM | 4682 | CB | VAL | B | 272 | 17.077 | 102.865 | 18.479 | 1.00 | 29.16 |
| ATOM | 4683 | CG1 | VAL | B | 272 | 16.785 | 101.520 | 19.139 | 1.00 | 26.10 |
| ATOM | 4684 | CG2 | VAL | B | 272 | 16.254 | 103.037 | 17.208 | 1.00 | 28.45 |
| ATOM | 4685 | C | VAL | B | 272 | 17.439 | 103.673 | 20.811 | 1.00 | 29.05 |
| ATOM | 4686 | O | VAL | B | 272 | 16.729 | 103.343 | 21.753 | 1.00 | 30.91 |
| ATOM | 4687 | N | LEU | B | 273 | 18.763 | 103.706 | 20.885 | 1.00 | 29.13 |
| ATOM | 4688 | CA | LEU | B | 273 | 19.475 | 103.352 | 22.106 | 1.00 | 28.98 |
| ATOM | 4689 | CB | LEU | B | 273 | 20.936 | 103.024 | 21.786 | 1.00 | 27.13 |
| ATOM | 4690 | CG | LEU | B | 273 | 21.202 | 101.678 | 21.103 | 1.00 | 25.76 |
| ATOM | 4691 | CD1 | LEU | B | 273 | 22.652 | 101.264 | 21.268 | 1.00 | 24.23 |
| ATOM | 4692 | CD2 | LEU | B | 273 | 20.273 | 100.605 | 21.641 | 1.00 | 27.25 |
| ATOM | 4693 | C | LEU | B | 273 | 19.413 | 104.463 | 23.156 | 1.00 | 31.27 |
| ATOM | 4694 | O | LEU | B | 273 | 19.689 | 104.233 | 24.344 | 1.00 | 33.10 |
| ATOM | 4695 | N | GLY | B | 274 | 19.081 | 105.664 | 22.709 | 1.00 | 30.29 |
| ATOM | 4696 | CA | GLY | B | 274 | 19.030 | 106.793 | 23.599 | 1.00 | 28.69 |
| ATOM | 4697 | C | GLY | B | 274 | 20.424 | 107.311 | 23.816 | 1.00 | 28.22 |
| ATOM | 4698 | O | GLY | B | 274 | 21.393 | 106.622 | 23.494 | 1.00 | 27.85 |
| ATOM | 4699 | N | THR | B | 275 | 20.548 | 108.513 | 24.353 | 1.00 | 28.54 |
| ATOM | 4700 | CA | THR | B | 275 | 21.874 | 109.068 | 24.585 | 1.00 | 29.17 |
| ATOM | 4701 | CB | THR | B | 275 | 21.782 | 110.445 | 25.229 | 1.00 | 28.46 |
| ATOM | 4702 | OG1 | THR | B | 275 | 20.977 | 111.305 | 24.448 | 1.00 | 31.45 |
| ATOM | 4703 | CG2 | THR | B | 275 | 23.127 | 111.112 | 25.418 | 1.00 | 27.39 |
| ATOM | 4704 | C | THR | B | 275 | 22.723 | 108.132 | 25.439 | 1.00 | 29.25 |
| ATOM | 4705 | O | THR | B | 275 | 22.255 | 107.618 | 26.455 | 1.00 | 30.69 |
| ATOM | 4706 | N | PRO | B | 276 | 23.989 | 107.893 | 25.036 | 1.00 | 29.38 |
| ATOM | 4707 | CD | PRO | B | 276 | 24.626 | 108.455 | 23.832 | 1.00 | 27.67 |
| ATOM | 4708 | CA | PRO | B | 276 | 24.897 | 107.021 | 25.778 | 1.00 | 28.22 |
| ATOM | 4709 | CB | PRO | B | 276 | 26.006 | 106.733 | 24.770 | 1.00 | 27.80 |
| ATOM | 4710 | CG | PRO | B | 276 | 26.039 | 107.941 | 23.901 | 1.00 | 24.60 |
| ATOM | 4711 | C | PRO | B | 276 | 25.462 | 107.752 | 26.996 | 1.00 | 30.33 |
| ATOM | 4712 | O | PRO | B | 276 | 25.782 | 108.942 | 26.918 | 1.00 | 28.30 |
| ATOM | 4713 | N | THR | B | 277 | 25.564 | 107.036 | 28.122 | 1.00 | 30.45 |
| ATOM | 4714 | CA | THR | B | 277 | 26.072 | 107.619 | 29.354 | 1.00 | 30.41 |
| ATOM | 4715 | CB | THR | B | 277 | 25.700 | 106.767 | 30.576 | 1.00 | 29.19 |
| ATOM | 4716 | OG1 | THR | B | 277 | 26.399 | 105.542 | 30.593 | 1.00 | 29.41 |
| ATOM | 4717 | CG2 | THR | B | 277 | 24.222 | 106.467 | 30.690 | 1.00 | 28.20 |
| ATOM | 4718 | C | THR | B | 277 | 27.571 | 107.845 | 29.282 | 1.00 | 31.58 |
| ATOM | 4719 | O | THR | B | 277 | 28.264 | 107.203 | 28.490 | 1.00 | 30.78 |
| ATOM | 4720 | N | ARG | B | 278 | 28.063 | 108.773 | 30.111 | 1.00 | 32.07 |
| ATOM | 4721 | CA | ARG | B | 278 | 29.485 | 109.104 | 30.148 | 1.00 | 33.38 |
| ATOM | 4722 | CB | ARG | B | 278 | 29.761 | 110.140 | 31.245 | 1.00 | 36.19 |
| ATOM | 4723 | CG | ARG | B | 278 | 29.811 | 109.553 | 32.648 | 1.00 | 40.29 |
| ATOM | 4724 | CD | ARG | B | 278 | 28.417 | 109.370 | 33.243 | 1.00 | 44.36 |
| ATOM | 4725 | NE | ARG | B | 278 | 28.111 | 107.962 | 33.513 | 1.00 | 44.31 |
| ATOM | 4726 | CZ | ARG | B | 278 | 26.871 | 107.469 | 33.607 | 1.00 | 46.69 |
| ATOM | 4727 | NH1 | ARG | B | 278 | 25.808 | 108.271 | 33.485 | 1.00 | 47.71 |
| ATOM | 4728 | NH2 | ARG | B | 278 | 26.690 | 106.167 | 33.831 | 1.00 | 46.93 |
| ATOM | 4729 | C | ARG | B | 278 | 30.340 | 107.855 | 30.356 | 1.00 | 32.74 |
| ATOM | 4730 | O | ARG | B | 278 | 31.452 | 107.760 | 29.840 | 1.00 | 34.25 |
| ATOM | 4731 | N | GLU | B | 279 | 29.811 | 106.890 | 31.101 | 1.00 | 32.02 |
| ATOM | 4732 | CA | GLU | B | 279 | 30.529 | 105.650 | 31.348 | 1.00 | 31.30 |
| ATOM | 4733 | CB | GLU | B | 279 | 29.874 | 104.848 | 32.482 | 1.00 | 31.99 |
| ATOM | 4734 | CG | GLU | B | 279 | 30.599 | 103.550 | 32.841 | 1.00 | 35.58 |
| ATOM | 4735 | CD | GLU | B | 279 | 32.113 | 103.698 | 32.960 | 1.00 | 36.93 |
| ATOM | 4736 | OE1 | GLU | B | 279 | 32.825 | 102.674 | 32.834 | 1.00 | 38.36 |
| ATOM | 4737 | OE2 | GLU | B | 279 | 32.589 | 104.831 | 33.183 | 1.00 | 39.38 |
| ATOM | 4738 | C | GLU | B | 279 | 30.601 | 104.817 | 30.068 | 1.00 | 28.54 |
| ATOM | 4739 | O | GLU | B | 279 | 31.653 | 104.304 | 29.722 | 1.00 | 29.69 |
| ATOM | 4740 | N | GLN | B | 280 | 29.471 | 104.709 | 29.371 | 1.00 | 27.39 |
| ATOM | 4741 | CA | GLN | B | 280 | 29.390 | 103.959 | 28.115 | 1.00 | 26.77 |
| ATOM | 4742 | CB | GLN | B | 280 | 27.947 | 103.942 | 27.595 | 1.00 | 24.23 |
| ATOM | 4743 | CG | GLN | B | 280 | 27.039 | 102.979 | 28.355 | 1.00 | 21.13 |
| ATOM | 4744 | CD | GLN | B | 280 | 25.564 | 103.172 | 28.073 | 1.00 | 18.64 |
| ATOM | 4745 | OE1 | GLN | B | 280 | 25.120 | 104.256 | 27.712 | 1.00 | 21.44 |
| ATOM | 4746 | NE2 | GLN | B | 280 | 24.789 | 102.109 | 28.242 | 1.00 | 22.07 |
| ATOM | 4747 | C | GLN | B | 280 | 30.356 | 104.535 | 27.077 | 1.00 | 27.30 |
| ATOM | 4748 | O | GLN | B | 280 | 31.039 | 103.787 | 26.372 | 1.00 | 28.93 |
| ATOM | 4749 | N | ILE | B | 281 | 30.441 | 105.868 | 27.023 | 1.00 | 28.72 |
| ATOM | 4750 | CA | ILE | B | 281 | 31.350 | 106.555 | 26.106 | 1.00 | 28.63 |
| ATOM | 4751 | CB | ILE | B | 281 | 31.120 | 108.079 | 26.100 | 1.00 | 29.61 |
| ATOM | 4752 | CG2 | ILE | B | 281 | 32.235 | 108.798 | 25.344 | 1.00 | 30.25 |
| ATOM | 4753 | CG1 | ILE | B | 281 | 29.758 | 108.407 | 25.489 | 1.00 | 28.88 |
| ATOM | 4754 | CD1 | ILE | B | 281 | 29.439 | 109.885 | 25.460 | 1.00 | 28.04 |
| ATOM | 4755 | C | ILE | B | 281 | 32.809 | 106.261 | 26.445 | 1.00 | 30.30 |
| ATOM | 4756 | O | ILE | B | 281 | 33.652 | 106.142 | 25.549 | 1.00 | 30.65 |
| ATOM | 4757 | N | ARG | B | 282 | 33.109 | 106.138 | 27.739 | 1.00 | 30.28 |
| ATOM | 4758 | CA | ARG | B | 282 | 34.470 | 105.848 | 28.181 | 1.00 | 29.89 |
| ATOM | 4759 | CB | ARG | B | 282 | 34.605 | 106.070 | 29.700 | 1.00 | 33.28 |
| ATOM | 4760 | CG | ARG | B | 282 | 35.923 | 106.718 | 30.111 | 1.00 | 35.52 |
| ATOM | 4761 | CD | ARG | B | 282 | 35.700 | 107.947 | 30.990 | 1.00 | 40.01 |
| ATOM | 4762 | NE | ARG | B | 282 | 36.769 | 108.944 | 30.842 | 1.00 | 41.68 |
| ATOM | 4763 | CZ | ARG | B | 282 | 38.015 | 108.791 | 31.316 | 1.00 | 44.99 |
| ATOM | 4764 | NH1 | ARG | B | 282 | 38.374 | 107.669 | 31.946 | 1.00 | 45.52 |
| ATOM | 4765 | NH2 | ARG | B | 282 | 38.911 | 109.763 | 31.150 | 1.00 | 46.24 |
| ATOM | 4766 | C | ARG | B | 282 | 34.855 | 104.413 | 27.816 | 1.00 | 28.46 |
| ATOM | 4767 | O | ARG | B | 282 | 36.016 | 104.111 | 27.549 | 1.00 | 28.25 |
| ATOM | 4768 | N | GLU | B | 283 | 33.869 | 103.536 | 27.794 | 1.00 | 28.29 |
| ATOM | 4769 | CA | GLU | B | 283 | 34.099 | 102.143 | 27.448 | 1.00 | 30.36 |
| ATOM | 4770 | CB | GLU | B | 283 | 32.931 | 101.276 | 27.929 | 1.00 | 30.84 |
| ATOM | 4771 | CG | GLU | B | 283 | 32.371 | 101.707 | 29.277 | 1.00 | 34.73 |
| ATOM | 4772 | CD | GLU | B | 283 | 31.757 | 100.572 | 30.063 | 1.00 | 35.22 |
| ATOM | 4773 | OE1 | GLU | B | 283 | 30.531 | 100.604 | 30.287 | 1.00 | 35.59 |
| ATOM | 4774 | OE2 | GLU | B | 283 | 32.505 | 99.654 | 30.460 | 1.00 | 38.74 |
| ATOM | 4775 | C | GLU | B | 283 | 34.314 | 101.983 | 25.938 | 1.00 | 30.59 |
| ATOM | 4776 | O | GLU | B | 283 | 35.030 | 101.083 | 25.494 | 1.00 | 30.56 |
| ATOM | 4777 | N | MET | B | 284 | 33.696 | 102.869 | 25.153 | 1.00 | 31.90 |
| ATOM | 4778 | CA | MET | B | 284 | 33.824 | 102.828 | 23.694 | 1.00 | 33.07 |
| ATOM | 4779 | CB | MET | B | 284 | 32.766 | 103.709 | 23.010 | 1.00 | 31.27 |
| ATOM | 4780 | CG | MET | B | 284 | 31.336 | 103.320 | 23.397 | 1.00 | 28.45 |
| ATOM | 4781 | SD | MET | B | 284 | 30.074 | 103.769 | 22.183 | 1.00 | 28.71 |
| ATOM | 4782 | CE | MET | B | 284 | 29.055 | 104.901 | 23.120 | 1.00 | 28.37 |
| ATOM | 4783 | C | MET | B | 284 | 35.274 | 103.066 | 23.209 | 1.00 | 35.36 |
| ATOM | 4784 | O | MET | B | 284 | 35.627 | 102.713 | 22.095 | 1.00 | 36.19 |
| ATOM | 4785 | N | ASN | B | 285 | 36.126 | 103.628 | 24.096 | 1.00 | 40.09 |
| ATOM | 4786 | CA | ASN | B | 285 | 37.565 | 103.878 | 23.885 | 1.00 | 41.52 |
| ATOM | 4787 | CB | ASN | B | 285 | 38.228 | 102.601 | 23.372 | 1.00 | 41.35 |
| ATOM | 4788 | CG | ASN | B | 285 | 39.010 | 101.810 | 24.425 | 1.00 | 41.10 |
| ATOM | 4789 | OD1 | ASN | B | 285 | 38.641 | 100.687 | 24.743 | 1.00 | 39.10 |
| ATOM | 4790 | ND2 | ASN | B | 285 | 40.070 | 102.412 | 24.949 | 1.00 | 39.99 |
| ATOM | 4791 | C | ASN | B | 285 | 37.889 | 105.060 | 22.969 | 1.00 | 43.22 |
| ATOM | 4792 | O | ASN | B | 285 | 38.927 | 105.089 | 22.316 | 1.00 | 44.35 |
| ATOM | 4793 | N | PRO | B | 286 | 36.949 | 106.038 | 22.916 | 1.00 | 42.77 |
| ATOM | 4794 | CD | PRO | B | 286 | 35.613 | 105.535 | 22.609 | 1.00 | 42.20 |
| ATOM | 4795 | CA | PRO | B | 286 | 37.137 | 107.295 | 22.114 | 1.00 | 43.36 |
| ATOM | 4796 | CB | PRO | B | 286 | 35.773 | 107.946 | 22.211 | 1.00 | 42.17 |
| ATOM | 4797 | CG | PRO | B | 286 | 34.880 | 106.742 | 22.145 | 1.00 | 41.91 |
| ATOM | 4798 | C | PRO | B | 286 | 38.281 | 108.205 | 22.503 | 1.00 | 45.30 |
| ATOM | 4799 | O | PRO | B | 286 | 39.043 | 108.744 | 21.711 | 1.00 | 46.02 |
| ATOM | 4800 | N | ASN | B | 287 | 38.364 | 108.297 | 23.816 | 1.00 | 46.61 |
| ATOM | 4801 | CA | ASN | B | 287 | 39.252 | 109.173 | 24.502 | 1.00 | 46.03 |
| ATOM | 4802 | CB | ASN | B | 287 | 40.695 | 109.000 | 24.024 | 1.00 | 46.35 |
| ATOM | 4803 | CG | ASN | B | 287 | 41.483 | 108.033 | 24.931 | 1.00 | 47.41 |
| ATOM | 4804 | OD1 | ASN | B | 287 | 42.702 | 108.016 | 24.913 | 1.00 | 47.27 |
| ATOM | 4805 | ND2 | ASN | B | 287 | 40.769 | 107.244 | 25.730 | 1.00 | 48.69 |
| ATOM | 4806 | C | ASN | B | 287 | 38.572 | 110.544 | 24.324 | 1.00 | 45.96 |
| ATOM | 4807 | O | ASN | B | 287 | 37.338 | 110.570 | 24.291 | 1.00 | 46.40 |
| ATOM | 4808 | N | TYR | B | 288 | 39.234 | 111.651 | 24.213 | 1.00 | 45.99 |
| ATOM | 4809 | CA | TYR | B | 288 | 38.481 | 112.942 | 24.243 | 1.00 | 47.00 |
| ATOM | 4810 | CB | TYR | B | 288 | 39.422 | 114.014 | 23.729 | 1.00 | 46.88 |
| ATOM | 4811 | CG | TYR | B | 288 | 39.402 | 114.302 | 22.265 | 1.00 | 47.95 |
| ATOM | 4812 | CD1 | TYR | B | 288 | 39.909 | 113.377 | 21.352 | 1.00 | 48.31 |
| ATOM | 4813 | CE1 | TYR | B | 288 | 39.914 | 113.642 | 19.993 | 1.00 | 47.74 |
| ATOM | 4814 | CD2 | TYR | B | 288 | 38.901 | 115.503 | 21.768 | 1.00 | 47.29 |
| ATOM | 4815 | CE2 | TYR | B | 288 | 38.902 | 115.775 | 20.411 | 1.00 | 47.45 |
| ATOM | 4816 | CZ | TYR | B | 288 | 39.410 | 114.841 | 19.528 | 1.00 | 48.12 |
| ATOM | 4817 | OH | TYR | B | 288 | 39.414 | 115.108 | 18.176 | 1.00 | 48.51 |
| ATOM | 4818 | C | TYR | B | 288 | 37.066 | 113.054 | 23.631 | 1.00 | 48.12 |
| ATOM | 4819 | O | TYR | B | 288 | 36.086 | 112.693 | 24.291 | 1.00 | 48.23 |
| ATOM | 4820 | N | THR | B | 289 | 36.934 | 113.523 | 22.398 | 1.00 | 49.01 |
| ATOM | 4821 | CA | THR | B | 289 | 35.644 | 113.609 | 21.677 | 1.00 | 50.53 |
| ATOM | 4822 | CB | THR | B | 289 | 35.006 | 112.211 | 21.583 | 1.00 | 49.77 |
| ATOM | 4823 | OG1 | THR | B | 289 | 35.396 | 111.568 | 20.363 | 1.00 | 47.97 |
| ATOM | 4824 | CG2 | THR | B | 289 | 33.491 | 112.312 | 21.629 | 1.00 | 48.53 |
| ATOM | 4825 | C | THR | B | 289 | 34.609 | 114.574 | 22.268 | 1.00 | 52.67 |
| ATOM | 4826 | O | THR | B | 289 | 33.934 | 114.254 | 23.256 | 1.00 | 53.68 |
| ATOM | 4827 | N | GLU | B | 290 | 34.502 | 115.768 | 21.629 | 1.00 | 54.04 |
| ATOM | 4828 | CA | GLU | B | 290 | 33.338 | 116.686 | 21.857 | 1.00 | 55.39 |
| ATOM | 4829 | CB | GLU | B | 290 | 33.580 | 117.961 | 21.042 | 1.00 | 56.12 |
| ATOM | 4830 | CG | GLU | B | 290 | 33.431 | 119.249 | 21.842 | 1.00 | 57.86 |
| ATOM | 4831 | CD | GLU | B | 290 | 34.524 | 119.422 | 22.886 | 1.00 | 59.39 |
| ATOM | 4832 | OE1 | GLU | B | 290 | 35.513 | 120.138 | 22.604 | 1.00 | 59.57 |
| ATOM | 4833 | OE2 | GLU | B | 290 | 34.391 | 118.840 | 23.987 | 1.00 | 60.09 |
| ATOM | 4834 | C | GLU | B | 290 | 31.900 | 116.202 | 21.684 | 1.00 | 56.39 |
| ATOM | 4835 | O | GLU | B | 290 | 31.408 | 116.094 | 20.558 | 1.00 | 55.69 |
| ATOM | 4836 | N | PHE | B | 291 | 31.251 | 115.905 | 22.809 | 1.00 | 57.38 |
| ATOM | 4837 | CA | PHE | B | 291 | 29.884 | 115.385 | 22.788 | 1.00 | 58.23 |
| ATOM | 4838 | CB | PHE | B | 291 | 29.463 | 114.915 | 24.213 | 1.00 | 58.40 |
| ATOM | 4839 | CG | PHE | B | 291 | 30.191 | 115.516 | 25.370 | 1.00 | 59.05 |
| ATOM | 4840 | CD1 | PHE | B | 291 | 29.563 | 116.464 | 26.167 | 1.00 | 59.39 |
| ATOM | 4841 | CD2 | PHE | B | 291 | 31.500 | 115.157 | 25.668 | 1.00 | 58.98 |
| ATOM | 4842 | CE1 | PHE | B | 291 | 30.226 | 117.041 | 27.239 | 1.00 | 59.94 |
| ATOM | 4843 | CE2 | PHE | B | 291 | 32.168 | 115.732 | 26.736 | 1.00 | 59.79 |
| ATOM | 4844 | CZ | PHE | B | 291 | 31.530 | 116.674 | 27.523 | 1.00 | 59.54 |
| ATOM | 4845 | C | PHE | B | 291 | 28.841 | 116.334 | 22.206 | 1.00 | 59.10 |
| ATOM | 4846 | O | PHE | B | 291 | 28.725 | 116.435 | 20.979 | 1.00 | 59.54 |
| ATOM | 4847 | N | LYS | B | 292 | 28.070 | 117.028 | 23.074 | 1.00 | 59.05 |
| ATOM | 4848 | CA | LYS | B | 292 | 26.995 | 117.952 | 22.657 | 1.00 | 59.28 |
| ATOM | 4849 | CB | LYS | B | 292 | 27.321 | 118.665 | 21.317 | 1.00 | 60.62 |
| ATOM | 4850 | CG | LYS | B | 292 | 26.818 | 117.941 | 20.064 | 1.00 | 60.64 |
| ATOM | 4851 | CD | LYS | B | 292 | 25.623 | 118.652 | 19.431 | 1.00 | 61.74 |
| ATOM | 4852 | CE | LYS | B | 292 | 24.974 | 117.805 | 18.342 | 1.00 | 61.38 |
| ATOM | 4853 | NZ | LYS | B | 292 | 24.644 | 116.430 | 18.826 | 1.00 | 63.08 |
| ATOM | 4854 | C | LYS | B | 292 | 25.657 | 117.203 | 22.558 | 1.00 | 58.54 |
| ATOM | 4855 | O | LYS | B | 292 | 24.670 | 117.727 | 22.031 | 1.00 | 59.16 |
| ATOM | 4856 | N | PHE | B | 293 | 25.645 | 115.965 | 23.058 | 1.00 | 57.33 |
| ATOM | 4857 | CA | PHE | B | 293 | 24.461 | 115.098 | 23.022 | 1.00 | 55.98 |
| ATOM | 4858 | CB | PHE | B | 293 | 24.787 | 113.698 | 23.560 | 1.00 | 54.70 |
| ATOM | 4859 | CG | PHE | B | 293 | 26.048 | 113.105 | 23.001 | 1.00 | 53.63 |
| ATOM | 4860 | CD1 | PHE | B | 293 | 26.233 | 112.994 | 21.631 | 1.00 | 53.34 |
| ATOM | 4861 | CD2 | PHE | B | 293 | 27.054 | 112.665 | 23.844 | 1.00 | 53.03 |
| ATOM | 4862 | CE1 | PHE | B | 293 | 27.397 | 112.455 | 21.114 | 1.00 | 52.99 |
| ATOM | 4863 | CE2 | PHE | B | 293 | 28.219 | 112.123 | 23.335 | 1.00 | 53.14 |
| ATOM | 4864 | CZ | PHE | B | 293 | 28.393 | 112.019 | 21.967 | 1.00 | 53.19 |
| ATOM | 4865 | C | PHE | B | 293 | 23.246 | 115.676 | 23.749 | 1.00 | 54.80 |
| ATOM | 4866 | O | PHE | B | 293 | 23.260 | 115.859 | 24.970 | 1.00 | 54.06 |
| ATOM | 4867 | N | PRO | B | 294 | 22.165 | 115.944 | 22.994 | 1.00 | 54.22 |
| ATOM | 4868 | CD | PRO | B | 294 | 22.067 | 115.735 | 21.547 | 1.00 | 54.04 |
| ATOM | 4869 | CA | PRO | B | 294 | 20.913 | 116.473 | 23.541 | 1.00 | 53.37 |
| ATOM | 4870 | CB | PRO | B | 294 | 20.163 | 116.990 | 22.296 | 1.00 | 54.04 |
| ATOM | 4871 | CG | PRO | B | 294 | 21.098 | 116.803 | 21.140 | 1.00 | 53.92 |
| ATOM | 4872 | C | PRO | B | 294 | 20.100 | 115.372 | 24.220 | 1.00 | 52.60 |
| ATOM | 4873 | O | PRO | B | 294 | 19.065 | 114.955 | 23.703 | 1.00 | 53.54 |
| ATOM | 4874 | N | GLN | B | 295 | 20.595 | 114.909 | 25.376 | 1.00 | 51.64 |
| ATOM | 4875 | CA | GLN | B | 295 | 19.978 | 113.844 | 26.186 | 1.00 | 50.54 |
| ATOM | 4876 | CB | GLN | B | 295 | 19.970 | 114.220 | 27.672 | 1.00 | 49.98 |
| ATOM | 4877 | CG | GLN | B | 295 | 21.056 | 113.536 | 28.497 | 1.00 | 48.79 |
| ATOM | 4878 | CD | GLN | B | 295 | 20.835 | 112.037 | 28.656 | 1.00 | 48.49 |
| ATOM | 4879 | OE1 | GLN | B | 295 | 19.740 | 111.523 | 28.414 | 1.00 | 49.23 |
| ATOM | 4880 | NE2 | GLN | B | 295 | 21.880 | 111.325 | 29.067 | 1.00 | 47.84 |
| ATOM | 4881 | C | GLN | B | 295 | 18.576 | 113.436 | 25.724 | 1.00 | 49.95 |
| ATOM | 4882 | O | GLN | B | 295 | 17.598 | 114.139 | 25.982 | 1.00 | 50.65 |
| ATOM | 4883 | N | ILE | B | 296 | 18.495 | 112.285 | 25.044 | 1.00 | 49.14 |
| ATOM | 4884 | CA | ILE | B | 296 | 17.226 | 111.755 | 24.541 | 1.00 | 47.84 |
| ATOM | 4885 | CB | ILE | B | 296 | 17.139 | 111.751 | 22.989 | 1.00 | 47.05 |
| ATOM | 4886 | CG2 | ILE | B | 296 | 18.317 | 112.482 | 22.362 | 1.00 | 46.60 |
| ATOM | 4887 | CG1 | ILE | B | 296 | 17.011 | 110.332 | 22.422 | 1.00 | 47.81 |
| ATOM | 4888 | CD1 | ILE | B | 296 | 15.633 | 110.022 | 21.879 | 1.00 | 47.64 |
| ATOM | 4889 | C | ILE | B | 296 | 16.928 | 110.370 | 25.129 | 1.00 | 47.17 |
| ATOM | 4890 | O | ILE | B | 296 | 17.835 | 109.662 | 25.572 | 1.00 | 47.85 |
| ATOM | 4891 | N | LYS | B | 297 | 15.650 | 110.008 | 25.163 | 1.00 | 46.24 |
| ATOM | 4892 | CA | LYS | B | 297 | 15.228 | 108.737 | 25.734 | 1.00 | 46.31 |
| ATOM | 4893 | CB | LYS | B | 297 | 13.770 | 108.819 | 26.195 | 1.00 | 47.40 |
| ATOM | 4894 | CG | LYS | B | 297 | 13.624 | 109.112 | 27.682 | 1.00 | 48.65 |
| ATOM | 4895 | CD | LYS | B | 297 | 13.321 | 110.582 | 27.938 | 1.00 | 49.80 |
| ATOM | 4896 | CE | LYS | B | 297 | 13.284 | 110.905 | 29.431 | 1.00 | 49.46 |
| ATOM | 4897 | NZ | LYS | B | 297 | 14.326 | 111.907 | 29.813 | 1.00 | 49.34 |
| ATOM | 4898 | C | LYS | B | 297 | 15.394 | 107.577 | 24.766 | 1.00 | 46.58 |
| ATOM | 4899 | O | LYS | B | 297 | 15.210 | 107.717 | 23.554 | 1.00 | 46.65 |
| ATOM | 4900 | N | ALA | B | 298 | 15.725 | 106.420 | 25.322 | 1.00 | 45.88 |
| ATOM | 4901 | CA | ALA | B | 298 | 15.898 | 105.221 | 24.529 | 1.00 | 45.60 |
| ATOM | 4902 | CB | ALA | B | 298 | 16.747 | 104.203 | 25.271 | 1.00 | 45.85 |
| ATOM | 4903 | C | ALA | B | 298 | 14.549 | 104.623 | 24.178 | 1.00 | 45.64 |
| ATOM | 4904 | O | ALA | B | 298 | 13.745 | 104.318 | 25.061 | 1.00 | 45.66 |
| ATOM | 4905 | N | HIS | B | 299 | 14.313 | 104.446 | 22.879 | 1.00 | 45.97 |
| ATOM | 4906 | CA | HIS | B | 299 | 13.073 | 103.860 | 22.397 | 1.00 | 44.58 |
| ATOM | 4907 | CB | HIS | B | 299 | 12.970 | 104.049 | 20.883 | 1.00 | 44.27 |
| ATOM | 4908 | CG | HIS | B | 299 | 11.687 | 103.567 | 20.292 | 1.00 | 44.81 |
| ATOM | 4909 | CD2 | HIS | B | 299 | 11.433 | 102.545 | 19.437 | 1.00 | 45.61 |
| ATOM | 4910 | ND1 | HIS | B | 299 | 10.469 | 104.151 | 20.559 | 1.00 | 44.53 |
| ATOM | 4911 | CE1 | HIS | B | 299 | 9.518 | 103.511 | 19.898 | 1.00 | 44.42 |
| ATOM | 4912 | NE2 | HIS | B | 299 | 10.077 | 102.533 | 19.208 | 1.00 | 44.70 |
| ATOM | 4913 | C | HIS | B | 299 | 13.066 | 102.374 | 22.760 | 1.00 | 44.19 |
| ATOM | 4914 | O | HIS | B | 299 | 13.877 | 101.599 | 22.245 | 1.00 | 44.88 |
| ATOM | 4915 | N | PRO | B | 300 | 12.166 | 101.963 | 23.677 | 1.00 | 43.75 |
| ATOM | 4916 | CD | PRO | B | 300 | 11.186 | 102.819 | 24.353 | 1.00 | 42.82 |
| ATOM | 4917 | CA | PRO | B | 300 | 12.068 | 100.575 | 24.136 | 1.00 | 42.83 |
| ATOM | 4918 | CB | PRO | B | 300 | 10.711 | 100.515 | 24.856 | 1.00 | 42.70 |
| ATOM | 4919 | CG | PRO | B | 300 | 10.079 | 101.858 | 24.663 | 1.00 | 42.34 |
| ATOM | 4920 | C | PRO | B | 300 | 12.133 | 99.563 | 22.989 | 1.00 | 42.56 |
| ATOM | 4921 | O | PRO | B | 300 | 11.382 | 99.659 | 22.011 | 1.00 | 42.03 |
| ATOM | 4922 | N | TRP | B | 301 | 13.050 | 98.602 | 23.118 | 1.00 | 41.90 |
| ATOM | 4923 | CA | TRP | B | 301 | 13.253 | 97.562 | 22.110 | 1.00 | 42.83 |
| ATOM | 4924 | CB | TRP | B | 301 | 14.209 | 96.480 | 22.629 | 1.00 | 41.84 |
| ATOM | 4925 | CG | TRP | B | 301 | 15.664 | 96.775 | 22.382 | 1.00 | 39.53 |
| ATOM | 4926 | CD2 | TRP | B | 301 | 16.288 | 97.049 | 21.119 | 1.00 | 39.21 |
| ATOM | 4927 | CE2 | TRP | B | 301 | 17.656 | 97.263 | 21.366 | 1.00 | 39.10 |
| ATOM | 4928 | CE3 | TRP | B | 301 | 15.820 | 97.139 | 19.806 | 1.00 | 38.84 |
| ATOM | 4929 | CD1 | TRP | B | 301 | 16.660 | 96.828 | 23.314 | 1.00 | 39.66 |
| ATOM | 4930 | NE1 | TRP | B | 301 | 17.858 | 97.124 | 22.716 | 1.00 | 37.86 |
| ATOM | 4931 | CZ2 | TRP | B | 301 | 18.561 | 97.554 | 20.347 | 1.00 | 39.02 |
| ATOM | 4932 | CZ3 | TRP | B | 301 | 16.718 | 97.428 | 18.798 | 1.00 | 38.68 |
| ATOM | 4933 | CH2 | TRP | B | 301 | 18.072 | 97.635 | 19.073 | 1.00 | 38.45 |
| ATOM | 4934 | C | TRP | B | 301 | 11.929 | 96.945 | 21.634 | 1.00 | 43.72 |
| ATOM | 4935 | O | TRP | B | 301 | 11.745 | 96.702 | 20.439 | 1.00 | 43.72 |
| ATOM | 4936 | N | THR | B | 302 | 11.011 | 96.704 | 22.572 | 1.00 | 44.52 |
| ATOM | 4937 | CA | THR | B | 302 | 9.703 | 96.124 | 22.260 | 1.00 | 45.04 |
| ATOM | 4938 | CB | THR | B | 302 | 8.907 | 95.844 | 23.545 | 1.00 | 45.51 |
| ATOM | 4939 | OG1 | THR | B | 302 | 7.551 | 95.537 | 23.254 | 1.00 | 45.99 |
| ATOM | 4940 | CG2 | THR | B | 302 | 8.926 | 96.984 | 24.545 | 1.00 | 44.89 |
| ATOM | 4941 | C | THR | B | 302 | 8.883 | 97.008 | 21.308 | 1.00 | 45.16 |
| ATOM | 4942 | O | THR | B | 302 | 8.097 | 96.498 | 20.502 | 1.00 | 45.19 |
| ATOM | 4943 | N | LYS | B | 303 | 9.061 | 98.327 | 21.401 | 1.00 | 44.79 |
| ATOM | 4944 | CA | LYS | B | 303 | 8.327 | 99.257 | 20.541 | 1.00 | 44.92 |
| ATOM | 4945 | CB | LYS | B | 303 | 8.124 | 100.605 | 21.251 | 1.00 | 46.86 |
| ATOM | 4946 | CG | LYS | B | 303 | 6.664 | 101.044 | 21.336 | 1.00 | 49.87 |
| ATOM | 4947 | CD | LYS | B | 303 | 5.934 | 100.861 | 20.003 | 1.00 | 51.82 |
| ATOM | 4948 | CE | LYS | B | 303 | 5.317 | 102.165 | 19.496 | 1.00 | 52.70 |
| ATOM | 4949 | NZ | LYS | B | 303 | 4.646 | 101.983 | 18.168 | 1.00 | 52.78 |
| ATOM | 4950 | C | LYS | B | 303 | 9.049 | 99.465 | 19.198 | 1.00 | 43.33 |
| ATOM | 4951 | O | LYS | B | 303 | 8.497 | 100.069 | 18.276 | 1.00 | 42.39 |
| ATOM | 4952 | N | VAL | B | 304 | 10.276 | 98.956 | 19.094 | 1.00 | 41.74 |
| ATOM | 4953 | CA | VAL | B | 304 | 11.060 | 99.081 | 17.868 | 1.00 | 41.90 |
| ATOM | 4954 | CB | VAL | B | 304 | 12.546 | 98.714 | 18.095 | 1.00 | 41.47 |
| ATOM | 4955 | CG1 | VAL | B | 304 | 13.323 | 98.774 | 16.785 | 1.00 | 42.51 |
| ATOM | 4956 | CG2 | VAL | B | 304 | 13.180 | 99.628 | 19.136 | 1.00 | 40.62 |
| ATOM | 4957 | C | VAL | B | 304 | 10.490 | 98.189 | 16.766 | 1.00 | 41.97 |
| ATOM | 4958 | O | VAL | B | 304 | 10.239 | 98.644 | 15.651 | 1.00 | 42.22 |
| ATOM | 4959 | N | PHE | B | 305 | 10.300 | 96.917 | 17.090 | 1.00 | 41.01 |
| ATOM | 4960 | CA | PHE | B | 305 | 9.787 | 95.947 | 16.141 | 1.00 | 40.52 |
| ATOM | 4961 | CB | PHE | B | 305 | 10.460 | 94.596 | 16.352 | 1.00 | 37.42 |
| ATOM | 4962 | CG | PHE | B | 305 | 11.955 | 94.683 | 16.453 | 1.00 | 34.99 |
| ATOM | 4963 | CD1 | PHE | B | 305 | 12.572 | 94.848 | 17.684 | 1.00 | 32.56 |
| ATOM | 4964 | CD2 | PHE | B | 305 | 12.746 | 94.603 | 15.317 | 1.00 | 32.98 |
| ATOM | 4965 | CE1 | PHE | B | 305 | 13.945 | 94.931 | 17.783 | 1.00 | 31.01 |
| ATOM | 4966 | CE2 | PHE | B | 305 | 14.121 | 94.685 | 15.410 | 1.00 | 31.65 |
| ATOM | 4967 | CZ | PHE | B | 305 | 14.722 | 94.848 | 16.646 | 1.00 | 32.89 |
| ATOM | 4968 | C | PHE | B | 305 | 8.269 | 95.812 | 16.186 | 1.00 | 42.03 |
| ATOM | 4969 | O | PHE | B | 305 | 7.626 | 96.153 | 17.177 | 1.00 | 43.48 |
| ATOM | 4970 | N | ARG | B | 306 | 7.712 | 95.310 | 15.087 | 1.00 | 43.32 |
| ATOM | 4971 | CA | ARG | B | 306 | 6.273 | 95.115 | 14.952 | 1.00 | 44.86 |
| ATOM | 4972 | CB | ARG | B | 306 | 5.951 | 94.552 | 13.566 | 1.00 | 46.33 |
| ATOM | 4973 | CG | ARG | B | 306 | 5.778 | 95.620 | 12.493 | 1.00 | 50.23 |
| ATOM | 4974 | CD | ARG | B | 306 | 5.129 | 95.056 | 11.224 | 1.00 | 52.92 |
| ATOM | 4975 | NE | ARG | B | 306 | 5.371 | 93.616 | 11.070 | 1.00 | 55.18 |
| ATOM | 4976 | CZ | ARG | B | 306 | 6.529 | 93.092 | 10.663 | 1.00 | 55.49 |
| ATOM | 4977 | NH1 | ARG | B | 306 | 7.512 | 93.883 | 10.231 | 1.00 | 54.95 |
| ATOM | 4978 | NH2 | ARG | B | 306 | 6.696 | 91.771 | 10.671 | 1.00 | 55.57 |
| ATOM | 4979 | C | ARG | B | 306 | 5.730 | 94.165 | 16.016 | 1.00 | 44.89 |
| ATOM | 4980 | O | ARG | B | 306 | 6.452 | 93.317 | 16.528 | 1.00 | 44.17 |
| ATOM | 4981 | N | PRO | B | 307 | 4.430 | 94.285 | 16.344 | 1.00 | 45.67 |
| ATOM | 4982 | CD | PRO | B | 307 | 3.491 | 95.258 | 15.759 | 1.00 | 45.99 |
| ATOM | 4983 | CA | PRO | B | 307 | 3.785 | 93.418 | 17.331 | 1.00 | 46.08 |
| ATOM | 4984 | CB | PRO | B | 307 | 2.315 | 93.850 | 17.290 | 1.00 | 46.12 |
| ATOM | 4985 | CG | PRO | B | 307 | 2.324 | 95.214 | 16.699 | 1.00 | 45.88 |
| ATOM | 4986 | C | PRO | B | 307 | 3.895 | 91.958 | 16.911 | 1.00 | 47.24 |
| ATOM | 4987 | O | PRO | B | 307 | 3.956 | 91.655 | 15.718 | 1.00 | 48.31 |
| ATOM | 4988 | N | ARG | B | 308 | 3.923 | 91.065 | 17.899 | 1.00 | 47.16 |
| ATOM | 4989 | CA | ARG | B | 308 | 4.024 | 89.621 | 17.666 | 1.00 | 46.75 |
| ATOM | 4990 | CB | ARG | B | 308 | 3.070 | 89.157 | 16.549 | 1.00 | 50.04 |
| ATOM | 4991 | CG | ARG | B | 308 | 1.887 | 88.340 | 17.062 | 1.00 | 53.29 |
| ATOM | 4992 | CD | ARG | B | 308 | 2.099 | 86.836 | 16.875 | 1.00 | 57.25 |
| ATOM | 4993 | NE | ARG | B | 308 | 3.413 | 86.383 | 17.356 | 1.00 | 59.65 |
| ATOM | 4994 | CZ | ARG | B | 308 | 4.243 | 85.576 | 16.668 | 1.00 | 60.80 |
| ATOM | 4995 | NH1 | ARG | B | 308 | 3.924 | 85.128 | 15.446 | 1.00 | 61.02 |
| ATOM | 4996 | NH2 | ARG | B | 308 | 5.405 | 85.218 | 17.208 | 1.00 | 60.97 |
| ATOM | 4997 | C | ARG | B | 308 | 5.463 | 89.183 | 17.371 | 1.00 | 45.19 |
| ATOM | 4998 | O | ARG | B | 308 | 5.743 | 87.985 | 17.319 | 1.00 | 45.53 |
| ATOM | 4999 | N | THR | B | 309 | 6.385 | 90.141 | 17.222 | 1.00 | 42.87 |
| ATOM | 5000 | CA | THR | B | 309 | 7.788 | 89.791 | 16.994 | 1.00 | 40.94 |
| ATOM | 5001 | CB | THR | B | 309 | 8.664 | 91.050 | 16.914 | 1.00 | 39.67 |
| ATOM | 5002 | OG1 | THR | B | 309 | 8.368 | 91.817 | 15.767 | 1.00 | 38.33 |
| ATOM | 5003 | CG2 | THR | B | 309 | 10.150 | 90.765 | 16.911 | 1.00 | 37.91 |
| ATOM | 5004 | C | THR | B | 309 | 8.260 | 88.920 | 18.164 | 1.00 | 41.33 |
| ATOM | 5005 | O | THR | B | 309 | 8.044 | 89.279 | 19.329 | 1.00 | 42.36 |
| ATOM | 5006 | N | PRO | B | 310 | 8.890 | 87.760 | 17.888 | 1.00 | 39.38 |
| ATOM | 5007 | CD | PRO | B | 310 | 9.182 | 87.232 | 16.551 | 1.00 | 39.12 |
| ATOM | 5008 | CA | PRO | B | 310 | 9.362 | 86.853 | 18.938 | 1.00 | 37.84 |
| ATOM | 5009 | CB | PRO | B | 310 | 10.309 | 85.889 | 18.203 | 1.00 | 37.57 |
| ATOM | 5010 | CG | PRO | B | 310 | 10.409 | 86.397 | 16.797 | 1.00 | 38.47 |
| ATOM | 5011 | C | PRO | B | 310 | 10.100 | 87.595 | 20.057 | 1.00 | 35.75 |
| ATOM | 5012 | O | PRO | B | 310 | 11.048 | 88.331 | 19.803 | 1.00 | 35.84 |
| ATOM | 5013 | N | PRO | B | 311 | 9.676 | 87.405 | 21.322 | 1.00 | 35.09 |
| ATOM | 5014 | CD | PRO | B | 311 | 8.560 | 86.544 | 21.737 | 1.00 | 34.88 |
| ATOM | 5015 | CA | PRO | B | 311 | 10.315 | 88.059 | 22.473 | 1.00 | 33.20 |
| ATOM | 5016 | CB | PRO | B | 311 | 9.520 | 87.535 | 23.676 | 1.00 | 33.84 |
| ATOM | 5017 | CG | PRO | B | 311 | 8.225 | 87.088 | 23.095 | 1.00 | 34.61 |
| ATOM | 5018 | C | PRO | B | 311 | 11.786 | 87.673 | 22.581 | 1.00 | 30.46 |
| ATOM | 5019 | O | PRO | B | 311 | 12.619 | 88.486 | 22.972 | 1.00 | 31.06 |
| ATOM | 5020 | N | GLU | B | 312 | 12.099 | 86.436 | 22.198 | 1.00 | 29.66 |
| ATOM | 5021 | CA | GLU | B | 312 | 13.475 | 85.934 | 22.217 | 1.00 | 29.89 |
| ATOM | 5022 | CB | GLU | B | 312 | 13.505 | 84.444 | 21.857 | 1.00 | 33.20 |
| ATOM | 5023 | CG | GLU | B | 312 | 12.895 | 83.526 | 22.907 | 1.00 | 37.51 |
| ATOM | 5024 | CD | GLU | B | 312 | 11.375 | 83.517 | 22.899 | 1.00 | 40.54 |
| ATOM | 5025 | OE1 | GLU | B | 312 | 10.787 | 82.991 | 23.871 | 1.00 | 42.54 |
| ATOM | 5026 | OE2 | GLU | B | 312 | 10.767 | 84.030 | 21.926 | 1.00 | 42.20 |
| ATOM | 5027 | C | GLU | B | 312 | 14.360 | 86.729 | 21.249 | 1.00 | 27.38 |
| ATOM | 5028 | O | GLU | B | 312 | 15.516 | 87.016 | 21.549 | 1.00 | 27.56 |
| ATOM | 5029 | N | ALA | B | 313 | 13.795 | 87.094 | 20.093 | 1.00 | 26.37 |
| ATOM | 5030 | CA | ALA | B | 313 | 14.522 | 87.871 | 19.081 | 1.00 | 26.28 |
| ATOM | 5031 | CB | ALA | B | 313 | 13.734 | 87.919 | 17.773 | 1.00 | 25.20 |
| ATOM | 5032 | C | ALA | B | 313 | 14.813 | 89.281 | 19.593 | 1.00 | 24.09 |
| ATOM | 5033 | O | ALA | B | 313 | 15.907 | 89.804 | 19.407 | 1.00 | 26.54 |
| ATOM | 5034 | N | ILE | B | 314 | 13.834 | 89.868 | 20.264 | 1.00 | 23.59 |
| ATOM | 5035 | CA | ILE | B | 314 | 13.969 | 91.196 | 20.846 | 1.00 | 23.45 |
| ATOM | 5036 | CB | ILE | B | 314 | 12.623 | 91.678 | 21.407 | 1.00 | 23.44 |
| ATOM | 5037 | CG2 | ILE | B | 314 | 12.786 | 92.990 | 22.165 | 1.00 | 26.62 |
| ATOM | 5038 | CG1 | ILE | B | 314 | 11.615 | 91.830 | 20.262 | 1.00 | 24.35 |
| ATOM | 5039 | CD1 | ILE | B | 314 | 10.334 | 92.547 | 20.635 | 1.00 | 21.48 |
| ATOM | 5040 | C | ILE | B | 314 | 15.029 | 91.197 | 21.946 | 1.00 | 24.53 |
| ATOM | 5041 | O | ILE | B | 314 | 15.885 | 92.090 | 22.001 | 1.00 | 26.34 |
| ATOM | 5042 | N | ALA | B | 315 | 14.987 | 90.172 | 22.812 | 1.00 | 25.59 |
| ATOM | 5043 | CA | ALA | B | 315 | 15.958 | 90.036 | 23.902 | 1.00 | 23.39 |
| ATOM | 5044 | CB | ALA | B | 315 | 15.575 | 88.884 | 24.837 | 1.00 | 21.46 |
| ATOM | 5045 | C | ALA | B | 315 | 17.370 | 89.850 | 23.358 | 1.00 | 22.54 |
| ATOM | 5046 | O | ALA | B | 315 | 18.298 | 90.543 | 23.789 | 1.00 | 23.20 |
| ATOM | 5047 | N | LEU | B | 316 | 17.537 | 88.928 | 22.392 | 1.00 | 19.48 |
| ATOM | 5048 | CA | LEU | B | 316 | 18.860 | 88.708 | 21.788 | 1.00 | 19.69 |
| ATOM | 5049 | CB | LEU | B | 316 | 18.826 | 87.569 | 20.743 | 1.00 | 20.12 |
| ATOM | 5050 | CG | LEU | B | 316 | 20.087 | 87.365 | 19.872 | 1.00 | 18.08 |
| ATOM | 5051 | CD1 | LEU | B | 316 | 21.336 | 87.216 | 20.716 | 1.00 | 13.38 |
| ATOM | 5052 | CD2 | LEU | B | 316 | 19.926 | 86.150 | 18.961 | 1.00 | 18.18 |
| ATOM | 5053 | C | LEU | B | 316 | 19.381 | 90.001 | 21.155 | 1.00 | 17.97 |
| ATOM | 5054 | O | LEU | B | 316 | 20.558 | 90.325 | 21.261 | 1.00 | 20.62 |
| ATOM | 5055 | N | CYS | B | 317 | 18.492 | 90.735 | 20.505 | 1.00 | 19.40 |
| ATOM | 5056 | CA | CYS | B | 317 | 18.851 | 91.990 | 19.856 | 1.00 | 22.68 |
| ATOM | 5057 | CB | CYS | B | 317 | 17.699 | 92.511 | 19.011 | 1.00 | 24.28 |
| ATOM | 5058 | SG | CYS | B | 317 | 18.179 | 93.846 | 17.899 | 1.00 | 36.41 |
| ATOM | 5059 | C | CYS | B | 317 | 19.335 | 93.050 | 20.850 | 1.00 | 23.21 |
| ATOM | 5060 | O | CYS | B | 317 | 20.324 | 93.751 | 20.578 | 1.00 | 21.64 |
| ATOM | 5061 | N | SER | B | 318 | 18.660 | 93.157 | 22.024 | 1.00 | 23.27 |
| ATOM | 5062 | CA | SER | B | 318 | 19.084 | 94.128 | 23.054 | 1.00 | 21.97 |
| ATOM | 5063 | CB | SER | B | 318 | 18.075 | 94.219 | 24.224 | 1.00 | 23.01 |
| ATOM | 5064 | OG | SER | B | 318 | 18.004 | 93.001 | 24.970 | 1.00 | 20.21 |
| ATOM | 5065 | C | SER | B | 318 | 20.468 | 93.765 | 23.580 | 1.00 | 21.85 |
| ATOM | 5066 | O | SER | B | 318 | 21.228 | 94.637 | 23.994 | 1.00 | 22.20 |
| ATOM | 5067 | N | ARG | B | 319 | 20.787 | 92.463 | 23.558 | 1.00 | 21.62 |
| ATOM | 5068 | CA | ARG | B | 319 | 22.079 | 91.969 | 24.038 | 1.00 | 22.39 |
| ATOM | 5069 | CB | ARG | B | 319 | 21.937 | 90.550 | 24.589 | 1.00 | 22.99 |
| ATOM | 5070 | CG | ARG | B | 319 | 20.671 | 90.338 | 25.426 | 1.00 | 27.25 |
| ATOM | 5071 | CD | ARG | B | 319 | 20.836 | 90.757 | 26.888 | 1.00 | 25.70 |
| ATOM | 5072 | NE | ARG | B | 319 | 22.048 | 91.537 | 27.127 | 1.00 | 27.16 |
| ATOM | 5073 | CZ | ARG | B | 319 | 23.185 | 91.038 | 27.632 | 1.00 | 27.95 |
| ATOM | 5074 | NH1 | ARG | B | 319 | 23.258 | 89.769 | 28.023 | 1.00 | 26.91 |
| ATOM | 5075 | NH2 | ARG | B | 319 | 24.256 | 91.824 | 27.752 | 1.00 | 30.55 |
| ATOM | 5076 | C | ARG | B | 319 | 23.167 | 91.990 | 22.969 | 1.00 | 22.03 |
| ATOM | 5077 | O | ARG | B | 319 | 24.264 | 91.471 | 23.189 | 1.00 | 22.19 |
| ATOM | 5078 | N | LEU | B | 320 | 22.883 | 92.597 | 21.818 | 1.00 | 22.15 |
| ATOM | 5079 | CA | LEU | B | 320 | 23.883 | 92.671 | 20.753 | 1.00 | 18.94 |
| ATOM | 5080 | CB | LEU | B | 320 | 23.382 | 91.989 | 19.477 | 1.00 | 17.86 |
| ATOM | 5081 | CG | LEU | B | 320 | 23.205 | 90.487 | 19.563 | 1.00 | 16.03 |
| ATOM | 5082 | CD1 | LEU | B | 320 | 22.562 | 89.959 | 18.288 | 1.00 | 17.54 |
| ATOM | 5083 | CD2 | LEU | B | 320 | 24.538 | 89.802 | 19.838 | 1.00 | 16.79 |
| ATOM | 5084 | C | LEU | B | 320 | 24.218 | 94.096 | 20.462 | 1.00 | 16.70 |
| ATOM | 5085 | O | LEU | B | 320 | 25.366 | 94.449 | 20.351 | 1.00 | 20.20 |
| ATOM | 5086 | N | LEU | B | 321 | 23.202 | 94.913 | 20.342 | 1.00 | 17.99 |
| ATOM | 5087 | CA | LEU | B | 321 | 23.393 | 96.315 | 20.062 | 1.00 | 18.47 |
| ATOM | 5088 | CB | LEU | B | 321 | 22.238 | 96.825 | 19.195 | 1.00 | 17.40 |
| ATOM | 5089 | CG | LEU | B | 321 | 21.995 | 96.035 | 17.906 | 1.00 | 17.58 |
| ATOM | 5090 | CD1 | LEU | B | 321 | 20.591 | 96.270 | 17.404 | 1.00 | 16.19 |
| ATOM | 5091 | CD2 | LEU | B | 321 | 23.027 | 96.403 | 16.851 | 1.00 | 14.68 |
| ATOM | 5092 | C | LEU | B | 321 | 23.521 | 97.113 | 21.360 | 1.00 | 19.25 |
| ATOM | 5093 | O | LEU | B | 321 | 22.659 | 97.903 | 21.707 | 1.00 | 16.25 |
| ATOM | 5094 | N | GLU | B | 322 | 24.612 | 96.866 | 22.072 | 1.00 | 22.51 |
| ATOM | 5095 | CA | GLU | B | 322 | 24.893 | 97.511 | 23.354 | 1.00 | 23.76 |
| ATOM | 5096 | CB | GLU | B | 322 | 25.302 | 96.425 | 24.370 | 1.00 | 24.61 |
| ATOM | 5097 | CG | GLU | B | 322 | 25.028 | 96.766 | 25.829 | 1.00 | 30.02 |
| ATOM | 5098 | CD | GLU | B | 322 | 23.710 | 96.206 | 26.325 | 1.00 | 27.86 |
| ATOM | 5099 | OE1 | GLU | B | 322 | 23.597 | 94.974 | 26.433 | 1.00 | 30.28 |
| ATOM | 5100 | OE2 | GLU | B | 322 | 22.792 | 97.003 | 26.597 | 1.00 | 29.21 |
| ATOM | 5101 | C | GLU | B | 322 | 26.034 | 98.493 | 23.203 | 1.00 | 21.13 |
| ATOM | 5102 | O | GLU | B | 322 | 27.068 | 98.149 | 22.657 | 1.00 | 25.15 |
| ATOM | 5103 | N | TYR | B | 323 | 25.859 | 99.705 | 23.710 | 1.00 | 20.92 |
| ATOM | 5104 | CA | TYR | B | 323 | 26.915 | 100.715 | 23.636 | 1.00 | 21.89 |
| ATOM | 5105 | CB | TYR | B | 323 | 26.536 | 101.953 | 24.426 | 1.00 | 21.76 |
| ATOM | 5106 | CG | TYR | B | 323 | 25.653 | 102.895 | 23.676 | 1.00 | 23.58 |
| ATOM | 5107 | CD1 | TYR | B | 323 | 26.065 | 103.451 | 22.471 | 1.00 | 22.37 |
| ATOM | 5108 | CE1 | TYR | B | 323 | 25.255 | 104.328 | 21.786 | 1.00 | 22.17 |
| ATOM | 5109 | CD2 | TYR | B | 323 | 24.411 | 103.236 | 24.174 | 1.00 | 23.51 |
| ATOM | 5110 | CE2 | TYR | B | 323 | 23.595 | 104.109 | 23.495 | 1.00 | 25.70 |
| ATOM | 5111 | CZ | TYR | B | 323 | 24.022 | 104.653 | 22.302 | 1.00 | 24.73 |
| ATOM | 5112 | OH | TYR | B | 323 | 23.205 | 105.525 | 21.625 | 1.00 | 24.89 |
| ATOM | 5113 | C | TYR | B | 323 | 28.241 | 100.186 | 24.149 | 1.00 | 22.26 |
| ATOM | 5114 | O | TYR | B | 323 | 29.276 | 100.338 | 23.495 | 1.00 | 24.24 |
| ATOM | 5115 | N | THR | B | 324 | 28.220 | 99.572 | 25.333 | 1.00 | 21.86 |
| ATOM | 5116 | CA | THR | B | 324 | 29.443 | 99.038 | 25.915 | 1.00 | 19.75 |
| ATOM | 5117 | CB | THR | B | 324 | 29.270 | 98.807 | 27.419 | 1.00 | 19.32 |
| ATOM | 5118 | OG1 | THR | B | 324 | 28.860 | 100.009 | 28.035 | 1.00 | 18.31 |
| ATOM | 5119 | CG2 | THR | B | 324 | 30.531 | 98.334 | 28.113 | 1.00 | 15.93 |
| ATOM | 5120 | C | THR | B | 324 | 29.839 | 97.776 | 25.192 | 1.00 | 19.46 |
| ATOM | 5121 | O | THR | B | 324 | 29.105 | 96.794 | 25.215 | 1.00 | 22.97 |
| ATOM | 5122 | N | PRO | B | 325 | 30.974 | 97.805 | 24.472 | 1.00 | 20.22 |
| ATOM | 5123 | CD | PRO | B | 325 | 31.870 | 98.973 | 24.316 | 1.00 | 18.06 |
| ATOM | 5124 | CA | PRO | B | 325 | 31.428 | 96.663 | 23.665 | 1.00 | 18.58 |
| ATOM | 5125 | CB | PRO | B | 325 | 32.754 | 97.137 | 23.061 | 1.00 | 15.36 |
| ATOM | 5126 | CG | PRO | B | 325 | 32.652 | 98.627 | 23.073 | 1.00 | 15.96 |
| ATOM | 5127 | C | PRO | B | 325 | 31.586 | 95.364 | 24.452 | 1.00 | 20.01 |
| ATOM | 5128 | O | PRO | B | 325 | 31.262 | 94.280 | 23.936 | 1.00 | 17.38 |
| ATOM | 5129 | N | THR | B | 326 | 32.077 | 95.462 | 25.700 | 1.00 | 19.19 |
| ATOM | 5130 | CA | THR | B | 326 | 32.259 | 94.262 | 26.534 | 1.00 | 18.37 |
| ATOM | 5131 | CB | THR | B | 326 | 33.212 | 94.528 | 27.711 | 1.00 | 18.49 |
| ATOM | 5132 | OG1 | THR | B | 326 | 32.854 | 95.724 | 28.385 | 1.00 | 19.48 |
| ATOM | 5133 | CG2 | THR | B | 326 | 34.672 | 94.623 | 27.313 | 1.00 | 14.73 |
| ATOM | 5134 | C | THR | B | 326 | 30.925 | 93.700 | 27.035 | 1.00 | 16.62 |
| ATOM | 5135 | O | THR | B | 326 | 30.866 | 92.560 | 27.489 | 1.00 | 20.08 |
| ATOM | 5136 | N | ALA | B | 327 | 29.860 | 94.502 | 26.935 | 1.00 | 17.88 |
| ATOM | 5137 | CA | ALA | B | 327 | 28.508 | 94.108 | 27.366 | 1.00 | 17.42 |
| ATOM | 5138 | CB | ALA | B | 327 | 27.679 | 95.344 | 27.649 | 1.00 | 18.02 |
| ATOM | 5139 | C | ALA | B | 327 | 27.774 | 93.225 | 26.342 | 1.00 | 18.63 |
| ATOM | 5140 | O | ALA | B | 327 | 26.790 | 92.543 | 26.683 | 1.00 | 17.38 |
| ATOM | 5141 | N | ARG | B | 328 | 28.233 | 93.249 | 25.082 | 1.00 | 19.14 |
| ATOM | 5142 | CA | ARG | B | 328 | 27.602 | 92.458 | 24.016 | 1.00 | 15.59 |
| ATOM | 5143 | CB | ARG | B | 328 | 28.110 | 92.919 | 22.633 | 1.00 | 17.66 |
| ATOM | 5144 | CG | ARG | B | 328 | 27.776 | 94.355 | 22.308 | 1.00 | 15.25 |
| ATOM | 5145 | CD | ARG | B | 328 | 28.704 | 94.916 | 21.237 | 1.00 | 18.95 |
| ATOM | 5146 | NE | ARG | B | 328 | 28.488 | 96.349 | 21.048 | 1.00 | 19.03 |
| ATOM | 5147 | CZ | ARG | B | 328 | 29.428 | 97.187 | 20.660 | 1.00 | 14.77 |
| ATOM | 5148 | NH1 | ARG | B | 328 | 30.594 | 96.726 | 20.266 | 1.00 | 14.20 |
| ATOM | 5149 | NH2 | ARG | B | 328 | 29.191 | 98.491 | 20.648 | 1.00 | 14.60 |
| ATOM | 5150 | C | ARG | B | 328 | 27.910 | 90.982 | 24.169 | 1.00 | 11.42 |
| ATOM | 5151 | O | ARG | B | 328 | 29.028 | 90.611 | 24.489 | 1.00 | 11.88 |
| ATOM | 5152 | N | LEU | B | 329 | 26.935 | 90.135 | 23.876 | 1.00 | 15.38 |
| ATOM | 5153 | CA | LEU | B | 329 | 27.178 | 88.699 | 23.928 | 1.00 | 17.98 |
| ATOM | 5154 | CB | LEU | B | 329 | 25.959 | 87.908 | 23.414 | 1.00 | 17.20 |
| ATOM | 5155 | CG | LEU | B | 329 | 24.653 | 88.035 | 24.178 | 1.00 | 20.30 |
| ATOM | 5156 | CD1 | LEU | B | 329 | 23.594 | 87.124 | 23.574 | 1.00 | 21.47 |
| ATOM | 5157 | CD2 | LEU | B | 329 | 24.845 | 87.731 | 25.661 | 1.00 | 22.07 |
| ATOM | 5158 | C | LEU | B | 329 | 28.340 | 88.355 | 23.016 | 1.00 | 19.84 |
| ATOM | 5159 | O | LEU | B | 329 | 28.686 | 89.127 | 22.097 | 1.00 | 24.07 |
| ATOM | 5160 | N | THR | B | 330 | 28.906 | 87.177 | 23.211 | 1.00 | 20.62 |
| ATOM | 5161 | CA | THR | B | 330 | 29.963 | 86.720 | 22.333 | 1.00 | 20.50 |
| ATOM | 5162 | CB | THR | B | 330 | 31.009 | 85.887 | 23.065 | 1.00 | 20.77 |
| ATOM | 5163 | OG1 | THR | B | 330 | 30.442 | 84.714 | 23.609 | 1.00 | 24.50 |
| ATOM | 5164 | CG2 | THR | B | 330 | 31.752 | 86.626 | 24.153 | 1.00 | 24.53 |
| ATOM | 5165 | C | THR | B | 330 | 29.279 | 85.866 | 21.262 | 1.00 | 18.71 |
| ATOM | 5166 | O | THR | B | 330 | 28.158 | 85.406 | 21.472 | 1.00 | 19.35 |
| ATOM | 5167 | N | PRO | B | 331 | 29.898 | 85.649 | 20.099 | 1.00 | 19.52 |
| ATOM | 5168 | CD | PRO | B | 331 | 31.220 | 86.162 | 19.696 | 1.00 | 18.68 |
| ATOM | 5169 | CA | PRO | B | 331 | 29.269 | 84.844 | 19.046 | 1.00 | 18.22 |
| ATOM | 5170 | CB | PRO | B | 331 | 30.390 | 84.661 | 18.018 | 1.00 | 16.44 |
| ATOM | 5171 | CG | PRO | B | 331 | 31.249 | 85.864 | 18.210 | 1.00 | 20.49 |
| ATOM | 5172 | C | PRO | B | 331 | 28.800 | 83.506 | 19.608 | 1.00 | 15.48 |
| ATOM | 5173 | O | PRO | B | 331 | 27.659 | 83.139 | 19.447 | 1.00 | 19.60 |
| ATOM | 5174 | N | LEU | B | 332 | 29.680 | 82.800 | 20.290 | 1.00 | 18.96 |
| ATOM | 5175 | CA | LEU | B | 332 | 29.320 | 81.507 | 20.885 | 1.00 | 21.83 |
| ATOM | 5176 | CB | LEU | B | 332 | 30.532 | 80.813 | 21.489 | 1.00 | 22.46 |
| ATOM | 5177 | CG | LEU | B | 332 | 30.508 | 79.294 | 21.410 | 1.00 | 24.80 |
| ATOM | 5178 | CD1 | LEU | B | 332 | 30.219 | 78.838 | 19.982 | 1.00 | 28.10 |
| ATOM | 5179 | CD2 | LEU | B | 332 | 31.826 | 78.721 | 21.909 | 1.00 | 27.01 |
| ATOM | 5180 | C | LEU | B | 332 | 28.190 | 81.635 | 21.909 | 1.00 | 20.95 |
| ATOM | 5181 | O | LEU | B | 332 | 27.370 | 80.737 | 22.032 | 1.00 | 23.46 |
| ATOM | 5182 | N | GLU | B | 333 | 28.122 | 82.779 | 22.599 | 1.00 | 21.24 |
| ATOM | 5183 | CA | GLU | B | 333 | 27.046 | 83.035 | 23.563 | 1.00 | 20.71 |
| ATOM | 5184 | CB | GLU | B | 333 | 27.355 | 84.274 | 24.426 | 1.00 | 21.87 |
| ATOM | 5185 | CG | GLU | B | 333 | 28.187 | 83.989 | 25.660 | 1.00 | 20.42 |
| ATOM | 5186 | CD | GLU | B | 333 | 28.867 | 85.226 | 26.207 | 1.00 | 20.58 |
| ATOM | 5187 | OE1 | GLU | B | 333 | 29.944 | 85.079 | 26.817 | 1.00 | 24.35 |
| ATOM | 5188 | OE2 | GLU | B | 333 | 28.341 | 86.337 | 26.011 | 1.00 | 21.62 |
| ATOM | 5189 | C | GLU | B | 333 | 25.759 | 83.284 | 22.813 | 1.00 | 19.41 |
| ATOM | 5190 | O | GLU | B | 333 | 24.691 | 82.822 | 23.208 | 1.00 | 19.72 |
| ATOM | 5191 | N | ALA | B | 334 | 25.860 | 84.024 | 21.707 | 1.00 | 20.30 |
| ATOM | 5192 | CA | ALA | B | 334 | 24.676 | 84.307 | 20.904 | 1.00 | 17.94 |
| ATOM | 5193 | CB | ALA | B | 334 | 25.014 | 85.247 | 19.740 | 1.00 | 22.73 |
| ATOM | 5194 | C | ALA | B | 334 | 24.077 | 83.000 | 20.400 | 1.00 | 15.00 |
| ATOM | 5195 | O | ALA | B | 334 | 22.864 | 82.823 | 20.414 | 1.00 | 15.28 |
| ATOM | 5196 | N | CYS | B | 335 | 24.943 | 82.087 | 19.956 | 1.00 | 16.88 |
| ATOM | 5197 | CA | CYS | B | 335 | 24.503 | 80.790 | 19.439 | 1.00 | 19.80 |
| ATOM | 5198 | CB | CYS | B | 335 | 25.712 | 79.943 | 18.958 | 1.00 | 22.65 |
| ATOM | 5199 | SG | CYS | B | 335 | 26.495 | 80.419 | 17.358 | 1.00 | 25.33 |
| ATOM | 5200 | C | CYS | B | 335 | 23.692 | 80.022 | 20.502 | 1.00 | 22.56 |
| ATOM | 5201 | O | CYS | B | 335 | 22.717 | 79.338 | 20.177 | 1.00 | 23.57 |
| ATOM | 5202 | N | ALA | B | 336 | 24.109 | 80.134 | 21.777 | 1.00 | 23.39 |
| ATOM | 5203 | CA | ALA | B | 336 | 23.438 | 79.448 | 22.891 | 1.00 | 22.44 |
| ATOM | 5204 | CB | ALA | B | 336 | 24.364 | 79.323 | 24.090 | 1.00 | 24.40 |
| ATOM | 5205 | C | ALA | B | 336 | 22.138 | 80.122 | 23.300 | 1.00 | 22.49 |
| ATOM | 5206 | O | ALA | B | 336 | 21.348 | 79.537 | 24.045 | 1.00 | 23.53 |
| ATOM | 5207 | N | HIS | B | 337 | 21.908 | 81.351 | 22.809 | 1.00 | 22.20 |
| ATOM | 5208 | CA | HIS | B | 337 | 20.690 | 82.090 | 23.129 | 1.00 | 19.18 |
| ATOM | 5209 | CB | HIS | B | 337 | 20.677 | 83.462 | 22.476 | 1.00 | 17.44 |
| ATOM | 5210 | CG | HIS | B | 337 | 19.683 | 84.412 | 23.083 | 1.00 | 12.51 |
| ATOM | 5211 | CD2 | HIS | B | 337 | 19.854 | 85.444 | 23.951 | 1.00 | 10.60 |
| ATOM | 5212 | ND1 | HIS | B | 337 | 18.343 | 84.380 | 22.799 | 1.00 | 12.23 |
| ATOM | 5213 | CE1 | HIS | B | 337 | 17.724 | 85.349 | 23.456 | 1.00 | 11.82 |
| ATOM | 5214 | NE2 | HIS | B | 337 | 18.626 | 86.009 | 24.162 | 1.00 | 10.75 |
| ATOM | 5215 | C | HIS | B | 337 | 19.431 | 81.301 | 22.787 | 1.00 | 23.47 |
| ATOM | 5216 | O | HIS | B | 337 | 19.409 | 80.490 | 21.828 | 1.00 | 24.86 |
| ATOM | 5217 | N | SER | B | 338 | 18.391 | 81.516 | 23.595 | 1.00 | 21.84 |
| ATOM | 5218 | CA | SER | B | 338 | 17.124 | 80.824 | 23.446 | 1.00 | 22.52 |
| ATOM | 5219 | CB | SER | B | 338 | 16.243 | 81.045 | 24.674 | 1.00 | 21.81 |
| ATOM | 5220 | OG | SER | B | 338 | 15.732 | 82.352 | 24.688 | 1.00 | 26.16 |
| ATOM | 5221 | C | SER | B | 338 | 16.375 | 81.171 | 22.154 | 1.00 | 21.98 |
| ATOM | 5222 | O | SER | B | 338 | 15.437 | 80.480 | 21.778 | 1.00 | 20.29 |
| ATOM | 5223 | N | PHE | B | 339 | 16.800 | 82.215 | 21.455 | 1.00 | 24.14 |
| ATOM | 5224 | CA | PHE | B | 339 | 16.144 | 82.552 | 20.186 | 1.00 | 23.45 |
| ATOM | 5225 | CB | PHE | B | 339 | 16.711 | 83.863 | 19.615 | 1.00 | 23.33 |
| ATOM | 5226 | CG | PHE | B | 339 | 16.172 | 84.233 | 18.250 | 1.00 | 24.35 |
| ATOM | 5227 | CD1 | PHE | B | 339 | 17.034 | 84.532 | 17.202 | 1.00 | 24.89 |
| ATOM | 5228 | CD2 | PHE | B | 339 | 14.812 | 84.276 | 18.013 | 1.00 | 24.61 |
| ATOM | 5229 | CE1 | PHE | B | 339 | 16.548 | 84.868 | 15.946 | 1.00 | 22.40 |
| ATOM | 5230 | CE2 | PHE | B | 339 | 14.317 | 84.612 | 16.761 | 1.00 | 24.59 |
| ATOM | 5231 | CZ | PHE | B | 339 | 15.192 | 84.910 | 15.727 | 1.00 | 21.68 |
| ATOM | 5232 | C | PHE | B | 339 | 16.377 | 81.398 | 19.193 | 1.00 | 21.36 |
| ATOM | 5233 | O | PHE | B | 339 | 15.579 | 81.152 | 18.297 | 1.00 | 22.30 |
| ATOM | 5234 | N | PHE | B | 340 | 17.499 | 80.716 | 19.386 | 1.00 | 21.16 |
| ATOM | 5235 | CA | PHE | B | 340 | 17.941 | 79.611 | 18.561 | 1.00 | 21.89 |
| ATOM | 5236 | CB | PHE | B | 340 | 19.452 | 79.723 | 18.390 | 1.00 | 18.67 |
| ATOM | 5237 | CG | PHE | B | 340 | 19.864 | 81.007 | 17.720 | 1.00 | 17.61 |
| ATOM | 5238 | CD1 | PHE | B | 340 | 19.361 | 81.342 | 16.461 | 1.00 | 17.37 |
| ATOM | 5239 | CD2 | PHE | B | 340 | 20.747 | 81.876 | 18.331 | 1.00 | 16.89 |
| ATOM | 5240 | CE1 | PHE | B | 340 | 19.740 | 82.517 | 15.834 | 1.00 | 16.28 |
| ATOM | 5241 | CE2 | PHE | B | 340 | 21.131 | 83.055 | 17.710 | 1.00 | 16.48 |
| ATOM | 5242 | CZ | PHE | B | 340 | 20.626 | 83.376 | 16.456 | 1.00 | 16.07 |
| ATOM | 5243 | C | PHE | B | 340 | 17.552 | 78.222 | 19.084 | 1.00 | 24.76 |
| ATOM | 5244 | O | PHE | B | 340 | 18.009 | 77.206 | 18.532 | 1.00 | 25.73 |
| ATOM | 5245 | N | ASP | B | 341 | 16.699 | 78.163 | 20.121 | 1.00 | 25.74 |
| ATOM | 5246 | CA | ASP | B | 341 | 16.256 | 76.877 | 20.674 | 1.00 | 26.49 |
| ATOM | 5247 | CB | ASP | B | 341 | 15.383 | 77.066 | 21.923 | 1.00 | 26.51 |
| ATOM | 5248 | CG | ASP | B | 341 | 16.165 | 77.514 | 23.166 | 1.00 | 27.55 |
| ATOM | 5249 | OD1 | ASP | B | 341 | 17.397 | 77.289 | 23.232 | 1.00 | 24.76 |
| ATOM | 5250 | OD2 | ASP | B | 341 | 15.535 | 78.090 | 24.074 | 1.00 | 29.17 |
| ATOM | 5251 | C | ASP | B | 341 | 15.511 | 76.037 | 19.632 | 1.00 | 26.31 |
| ATOM | 5252 | O | ASP | B | 341 | 15.652 | 74.819 | 19.599 | 1.00 | 27.59 |
| ATOM | 5253 | N | GLU | B | 342 | 14.726 | 76.693 | 18.773 | 1.00 | 28.02 |
| ATOM | 5254 | CA | GLU | B | 342 | 13.984 | 75.989 | 17.723 | 1.00 | 27.38 |
| ATOM | 5255 | CB | GLU | B | 342 | 13.200 | 76.983 | 16.851 | 1.00 | 28.41 |
| ATOM | 5256 | CG | GLU | B | 342 | 11.835 | 76.474 | 16.404 | 1.00 | 30.01 |
| ATOM | 5257 | CD | GLU | B | 342 | 11.296 | 77.144 | 15.141 | 1.00 | 30.63 |
| ATOM | 5258 | OE1 | GLU | B | 342 | 10.401 | 76.558 | 14.499 | 1.00 | 33.14 |
| ATOM | 5259 | OE2 | GLU | B | 342 | 11.755 | 78.247 | 14.799 | 1.00 | 32.10 |
| ATOM | 5260 | C | GLU | B | 342 | 14.936 | 75.155 | 16.852 | 1.00 | 26.89 |
| ATOM | 5261 | O | GLU | B | 342 | 14.623 | 74.028 | 16.487 | 1.00 | 27.34 |
| ATOM | 5262 | N | LEU | B | 343 | 16.104 | 75.722 | 16.543 | 1.00 | 26.14 |
| ATOM | 5263 | CA | LEU | B | 343 | 17.121 | 75.056 | 15.734 | 1.00 | 26.03 |
| ATOM | 5264 | CB | LEU | B | 343 | 18.309 | 75.995 | 15.498 | 1.00 | 24.38 |
| ATOM | 5265 | CG | LEU | B | 343 | 18.008 | 77.353 | 14.863 | 1.00 | 23.80 |
| ATOM | 5266 | CD1 | LEU | B | 343 | 19.306 | 78.029 | 14.443 | 1.00 | 23.33 |
| ATOM | 5267 | CD2 | LEU | B | 343 | 17.074 | 77.199 | 13.670 | 1.00 | 23.09 |
| ATOM | 5268 | C | LEU | B | 343 | 17.622 | 73.760 | 16.382 | 1.00 | 27.12 |
| ATOM | 5269 | O | LEU | B | 343 | 17.940 | 72.788 | 15.681 | 1.00 | 26.66 |
| ATOM | 5270 | N | ARG | B | 344 | 17.710 | 73.753 | 17.717 | 1.00 | 27.38 |
| ATOM | 5271 | CA | ARG | B | 344 | 18.189 | 72.579 | 18.461 | 1.00 | 28.04 |
| ATOM | 5272 | CB | ARG | B | 344 | 18.639 | 72.987 | 19.873 | 1.00 | 26.77 |
| ATOM | 5273 | CG | ARG | B | 344 | 20.065 | 73.500 | 19.918 | 1.00 | 23.60 |
| ATOM | 5274 | CD | ARG | B | 344 | 20.372 | 74.201 | 21.229 | 1.00 | 20.43 |
| ATOM | 5275 | NE | ARG | B | 344 | 19.656 | 75.468 | 21.370 | 1.00 | 15.24 |
| ATOM | 5276 | CZ | ARG | B | 344 | 20.233 | 76.664 | 21.314 | 1.00 | 14.32 |
| ATOM | 5277 | NH1 | ARG | B | 344 | 21.514 | 76.784 | 21.011 | 1.00 | 16.66 |
| ATOM | 5278 | NH2 | ARG | B | 344 | 19.520 | 77.750 | 21.535 | 1.00 | 13.81 |
| ATOM | 5279 | C | ARG | B | 344 | 17.121 | 71.491 | 18.533 | 1.00 | 29.05 |
| ATOM | 5280 | O | ARG | B | 344 | 17.413 | 70.348 | 18.877 | 1.00 | 29.56 |
| ATOM | 5281 | N | ASP | B | 345 | 15.891 | 71.847 | 18.177 | 1.00 | 32.04 |
| ATOM | 5282 | CA | ASP | B | 345 | 14.780 | 70.905 | 18.159 | 1.00 | 34.68 |
| ATOM | 5283 | CB | ASP | B | 345 | 13.523 | 71.632 | 17.696 | 1.00 | 36.26 |
| ATOM | 5284 | CG | ASP | B | 345 | 12.250 | 70.882 | 17.999 | 1.00 | 36.87 |
| ATOM | 5285 | OD1 | ASP | B | 345 | 11.252 | 71.545 | 18.346 | 1.00 | 40.08 |
| ATOM | 5286 | OD2 | ASP | B | 345 | 12.244 | 69.641 | 17.877 | 1.00 | 38.07 |
| ATOM | 5287 | C | ASP | B | 345 | 15.116 | 69.773 | 17.189 | 1.00 | 36.77 |
| ATOM | 5288 | O | ASP | B | 345 | 15.513 | 70.030 | 16.055 | 1.00 | 37.90 |
| ATOM | 5289 | N | PRO | B | 346 | 15.001 | 68.509 | 17.630 | 1.00 | 38.35 |
| ATOM | 5290 | CD | PRO | B | 346 | 14.562 | 68.112 | 18.980 | 1.00 | 38.89 |
| ATOM | 5291 | CA | PRO | B | 346 | 15.342 | 67.346 | 16.805 | 1.00 | 38.95 |
| ATOM | 5292 | CB | PRO | B | 346 | 15.287 | 66.185 | 17.796 | 1.00 | 38.46 |
| ATOM | 5293 | CG | PRO | B | 346 | 14.329 | 66.639 | 18.836 | 1.00 | 38.21 |
| ATOM | 5294 | C | PRO | B | 346 | 14.379 | 67.098 | 15.652 | 1.00 | 40.21 |
| ATOM | 5295 | O | PRO | B | 346 | 14.730 | 66.413 | 14.696 | 1.00 | 40.40 |
| ATOM | 5296 | N | ASN | B | 347 | 13.171 | 67.642 | 15.751 | 1.00 | 41.66 |
| ATOM | 5297 | CA | ASN | B | 347 | 12.163 | 67.455 | 14.712 | 1.00 | 43.83 |
| ATOM | 5298 | CB | ASN | B | 347 | 10.814 | 67.114 | 15.348 | 1.00 | 45.27 |
| ATOM | 5299 | CG | ASN | B | 347 | 10.860 | 65.853 | 16.188 | 1.00 | 46.39 |
| ATOM | 5300 | OD1 | ASN | B | 347 | 10.326 | 65.815 | 17.298 | 1.00 | 47.81 |
| ATOM | 5301 | ND2 | ASN | B | 347 | 11.498 | 64.811 | 15.662 | 1.00 | 46.93 |
| ATOM | 5302 | C | ASN | B | 347 | 12.017 | 68.670 | 13.782 | 1.00 | 44.37 |
| ATOM | 5303 | O | ASN | B | 347 | 11.171 | 68.666 | 12.887 | 1.00 | 45.46 |
| ATOM | 5304 | N | VAL | B | 348 | 12.830 | 69.706 | 14.000 | 1.00 | 44.26 |
| ATOM | 5305 | CA | VAL | B | 348 | 12.770 | 70.918 | 13.187 | 1.00 | 44.40 |
| ATOM | 5306 | CB | VAL | B | 348 | 13.643 | 72.059 | 13.765 | 1.00 | 43.32 |
| ATOM | 5307 | CG1 | VAL | B | 348 | 15.106 | 71.863 | 13.399 | 1.00 | 41.21 |
| ATOM | 5308 | CG2 | VAL | B | 348 | 13.146 | 73.411 | 13.274 | 1.00 | 42.08 |
| ATOM | 5309 | C | VAL | B | 348 | 13.151 | 70.667 | 11.725 | 1.00 | 44.95 |
| ATOM | 5310 | O | VAL | B | 348 | 14.174 | 70.043 | 11.428 | 1.00 | 44.90 |
| ATOM | 5311 | N | LYS | B | 349 | 12.321 | 71.178 | 10.822 | 1.00 | 45.79 |
| ATOM | 5312 | CA | LYS | B | 349 | 12.554 | 71.042 | 9.388 | 1.00 | 46.82 |
| ATOM | 5313 | CB | LYS | B | 349 | 11.557 | 70.057 | 8.758 | 1.00 | 48.92 |
| ATOM | 5314 | CG | LYS | B | 349 | 11.517 | 68.692 | 9.431 | 1.00 | 51.59 |
| ATOM | 5315 | CD | LYS | B | 349 | 12.587 | 67.757 | 8.885 | 1.00 | 54.24 |
| ATOM | 5316 | CE | LYS | B | 349 | 12.601 | 66.433 | 9.638 | 1.00 | 56.09 |
| ATOM | 5317 | NZ | LYS | B | 349 | 13.905 | 65.718 | 9.507 | 1.00 | 58.50 |
| ATOM | 5318 | C | LYS | B | 349 | 12.418 | 72.393 | 8.700 | 1.00 | 46.09 |
| ATOM | 5319 | O | LYS | B | 349 | 11.781 | 73.314 | 9.227 | 1.00 | 45.54 |
| ATOM | 5320 | N | LEU | B | 350 | 12.996 | 72.494 | 7.507 | 1.00 | 45.35 |
| ATOM | 5321 | CA | LEU | B | 350 | 12.928 | 73.709 | 6.707 | 1.00 | 43.22 |
| ATOM | 5322 | CB | LEU | B | 350 | 13.900 | 73.582 | 5.535 | 1.00 | 43.49 |
| ATOM | 5323 | CG | LEU | B | 350 | 15.222 | 74.336 | 5.638 | 1.00 | 44.81 |
| ATOM | 5324 | CD1 | LEU | B | 350 | 15.783 | 74.263 | 7.050 | 1.00 | 45.17 |
| ATOM | 5325 | CD2 | LEU | B | 350 | 16.220 | 73.783 | 4.625 | 1.00 | 43.94 |
| ATOM | 5326 | C | LEU | B | 350 | 11.509 | 73.871 | 6.156 | 1.00 | 41.98 |
| ATOM | 5327 | O | LEU | B | 350 | 10.753 | 72.903 | 6.117 | 1.00 | 42.03 |
| ATOM | 5328 | N | PRO | B | 351 | 11.125 | 75.090 | 5.709 | 1.00 | 41.39 |
| ATOM | 5329 | CD | PRO | B | 351 | 11.947 | 76.307 | 5.710 | 1.00 | 41.42 |
| ATOM | 5330 | CA | PRO | B | 351 | 9.792 | 75.345 | 5.145 | 1.00 | 41.01 |
| ATOM | 5331 | CB | PRO | B | 351 | 9.748 | 76.866 | 4.959 | 1.00 | 40.02 |
| ATOM | 5332 | CG | PRO | B | 351 | 10.909 | 77.390 | 5.734 | 1.00 | 41.12 |
| ATOM | 5333 | C | PRO | B | 351 | 9.584 | 74.634 | 3.802 | 1.00 | 40.65 |
| ATOM | 5334 | O | PRO | B | 351 | 8.473 | 74.595 | 3.286 | 1.00 | 41.97 |
| ATOM | 5335 | N | ASN | B | 352 | 10.646 | 74.050 | 3.252 | 1.00 | 41.95 |
| ATOM | 5336 | CA | ASN | B | 352 | 10.540 | 73.316 | 1.988 | 1.00 | 43.67 |
| ATOM | 5337 | CB | ASN | B | 352 | 11.620 | 73.726 | 0.976 | 1.00 | 43.38 |
| ATOM | 5338 | CG | ASN | B | 352 | 13.044 | 73.417 | 1.414 | 1.00 | 44.84 |
| ATOM | 5339 | OD1 | ASN | B | 352 | 14.001 | 73.851 | 0.780 | 1.00 | 45.42 |
| ATOM | 5340 | ND2 | ASN | B | 352 | 13.199 | 72.672 | 2.498 | 1.00 | 44.86 |
| ATOM | 5341 | C | ASN | B | 352 | 10.529 | 71.798 | 2.213 | 1.00 | 44.56 |
| ATOM | 5342 | O | ASN | B | 352 | 10.550 | 71.024 | 1.255 | 1.00 | 44.91 |
| ATOM | 5343 | N | GLY | B | 353 | 10.475 | 71.382 | 3.492 | 1.00 | 45.59 |
| ATOM | 5344 | CA | GLY | B | 353 | 10.430 | 69.962 | 3.839 | 1.00 | 45.61 |
| ATOM | 5345 | C | GLY | B | 353 | 11.785 | 69.334 | 4.108 | 1.00 | 45.64 |
| ATOM | 5346 | O | GLY | B | 353 | 11.890 | 68.383 | 4.888 | 1.00 | 45.91 |
| ATOM | 5347 | N | ARG | B | 354 | 12.818 | 69.845 | 3.450 | 1.00 | 45.87 |
| ATOM | 5348 | CA | ARG | B | 354 | 14.168 | 69.320 | 3.607 | 1.00 | 46.82 |
| ATOM | 5349 | CB | ARG | B | 354 | 15.118 | 70.022 | 2.642 | 1.00 | 48.66 |
| ATOM | 5350 | CG | ARG | B | 354 | 15.879 | 69.068 | 1.739 | 1.00 | 51.69 |
| ATOM | 5351 | CD | ARG | B | 354 | 16.789 | 69.826 | 0.782 | 1.00 | 54.15 |
| ATOM | 5352 | NE | ARG | B | 354 | 16.023 | 70.611 | −0.188 | 1.00 | 55.74 |
| ATOM | 5353 | CZ | ARG | B | 354 | 16.471 | 70.956 | −1.402 | 1.00 | 56.23 |
| ATOM | 5354 | NH1 | ARG | B | 354 | 17.690 | 70.599 | −1.806 | 1.00 | 55.16 |
| ATOM | 5355 | NH2 | ARG | B | 354 | 15.693 | 71.668 | −2.210 | 1.00 | 56.28 |
| ATOM | 5356 | C | ARG | B | 354 | 14.677 | 69.490 | 5.036 | 1.00 | 45.85 |
| ATOM | 5357 | O | ARG | B | 354 | 14.109 | 70.244 | 5.824 | 1.00 | 46.19 |
| ATOM | 5358 | N | ASP | B | 355 | 15.763 | 68.790 | 5.354 | 1.00 | 44.37 |
| ATOM | 5359 | CA | ASP | B | 355 | 16.369 | 68.872 | 6.680 | 1.00 | 43.25 |
| ATOM | 5360 | CB | ASP | B | 355 | 17.386 | 67.737 | 6.896 | 1.00 | 43.57 |
| ATOM | 5361 | CG | ASP | B | 355 | 16.791 | 66.339 | 6.930 | 1.00 | 44.36 |
| ATOM | 5362 | OD1 | ASP | B | 355 | 17.545 | 65.382 | 6.667 | 1.00 | 43.84 |
| ATOM | 5363 | OD2 | ASP | B | 355 | 15.584 | 66.196 | 7.229 | 1.00 | 46.22 |
| ATOM | 5364 | C | ASP | B | 355 | 17.118 | 70.188 | 6.820 | 1.00 | 41.99 |
| ATOM | 5365 | O | ASP | B | 355 | 17.510 | 70.808 | 5.821 | 1.00 | 41.58 |
| ATOM | 5366 | N | THR | B | 356 | 17.354 | 70.595 | 8.057 | 1.00 | 39.86 |
| ATOM | 5367 | CA | THR | B | 356 | 18.100 | 71.811 | 8.291 | 1.00 | 36.95 |
| ATOM | 5368 | CB | THR | B | 356 | 18.016 | 72.233 | 9.762 | 1.00 | 36.53 |
| ATOM | 5369 | OG1 | THR | B | 356 | 18.489 | 71.203 | 10.615 | 1.00 | 35.15 |
| ATOM | 5370 | CG2 | THR | B | 356 | 16.626 | 72.620 | 10.205 | 1.00 | 34.96 |
| ATOM | 5371 | C | THR | B | 356 | 19.543 | 71.548 | 7.930 | 1.00 | 35.17 |
| ATOM | 5372 | O | THR | B | 356 | 20.012 | 70.418 | 8.058 | 1.00 | 34.12 |
| ATOM | 5373 | N | PRO | B | 357 | 20.272 | 72.576 | 7.484 | 1.00 | 34.19 |
| ATOM | 5374 | CD | PRO | B | 357 | 19.788 | 73.953 | 7.304 | 1.00 | 33.98 |
| ATOM | 5375 | CA | PRO | B | 357 | 21.680 | 72.425 | 7.131 | 1.00 | 34.20 |
| ATOM | 5376 | CB | PRO | B | 357 | 22.115 | 73.845 | 6.729 | 1.00 | 34.84 |
| ATOM | 5377 | CG | PRO | B | 357 | 21.057 | 74.755 | 7.256 | 1.00 | 34.63 |
| ATOM | 5378 | C | PRO | B | 357 | 22.496 | 71.937 | 8.331 | 1.00 | 35.28 |
| ATOM | 5379 | O | PRO | B | 357 | 21.939 | 71.558 | 9.371 | 1.00 | 35.46 |
| ATOM | 5380 | N | ALA | B | 358 | 23.812 | 71.957 | 8.189 | 1.00 | 34.83 |
| ATOM | 5381 | CA | ALA | B | 358 | 24.707 | 71.525 | 9.251 | 1.00 | 35.70 |
| ATOM | 5382 | CB | ALA | B | 358 | 26.117 | 71.327 | 8.712 | 1.00 | 35.68 |
| ATOM | 5383 | C | ALA | B | 358 | 24.728 | 72.501 | 10.437 | 1.00 | 36.22 |
| ATOM | 5384 | O | ALA | B | 358 | 24.979 | 73.702 | 10.285 | 1.00 | 37.18 |
| ATOM | 5385 | N | LEU | B | 359 | 24.505 | 71.945 | 11.612 | 1.00 | 34.98 |
| ATOM | 5386 | CA | LEU | B | 359 | 24.526 | 72.662 | 12.886 | 1.00 | 35.55 |
| ATOM | 5387 | CB | LEU | B | 359 | 23.135 | 73.171 | 13.288 | 1.00 | 34.14 |
| ATOM | 5388 | CG | LEU | B | 359 | 22.245 | 73.728 | 12.175 | 1.00 | 35.54 |
| ATOM | 5389 | CD1 | LEU | B | 359 | 20.775 | 73.629 | 12.559 | 1.00 | 35.56 |
| ATOM | 5390 | CD2 | LEU | B | 359 | 22.617 | 75.169 | 11.852 | 1.00 | 35.80 |
| ATOM | 5391 | C | LEU | B | 359 | 25.024 | 71.670 | 13.922 | 1.00 | 36.61 |
| ATOM | 5392 | O | LEU | B | 359 | 24.878 | 70.459 | 13.719 | 1.00 | 39.28 |
| ATOM | 5393 | N | PHE | B | 360 | 25.618 | 72.151 | 15.014 | 1.00 | 35.74 |
| ATOM | 5394 | CA | PHE | B | 360 | 26.140 | 71.244 | 16.070 | 1.00 | 34.10 |
| ATOM | 5395 | CB | PHE | B | 360 | 25.101 | 70.202 | 16.508 | 1.00 | 29.33 |
| ATOM | 5396 | CG | PHE | B | 360 | 23.680 | 70.666 | 16.405 | 1.00 | 26.36 |
| ATOM | 5397 | CD1 | PHE | B | 360 | 22.752 | 69.925 | 15.695 | 1.00 | 23.97 |
| ATOM | 5398 | CD2 | PHE | B | 360 | 23.275 | 71.847 | 17.005 | 1.00 | 24.93 |
| ATOM | 5399 | CE1 | PHE | B | 360 | 21.446 | 70.351 | 15.579 | 1.00 | 21.97 |
| ATOM | 5400 | CE2 | PHE | B | 360 | 21.969 | 72.277 | 16.896 | 1.00 | 22.81 |
| ATOM | 5401 | CZ | PHE | B | 360 | 21.051 | 71.525 | 16.180 | 1.00 | 22.58 |
| ATOM | 5402 | C | PHE | B | 360 | 27.437 | 70.555 | 15.638 | 1.00 | 33.93 |
| ATOM | 5403 | O | PHE | B | 360 | 28.007 | 69.749 | 16.371 | 1.00 | 33.19 |
| ATOM | 5404 | N | ASN | B | 361 | 27.899 | 70.912 | 14.447 | 1.00 | 34.31 |
| ATOM | 5405 | CA | ASN | B | 361 | 29.133 | 70.399 | 13.858 | 1.00 | 34.36 |
| ATOM | 5406 | CB | ASN | B | 361 | 29.057 | 70.666 | 12.340 | 1.00 | 36.65 |
| ATOM | 5407 | CG | ASN | B | 361 | 29.837 | 69.677 | 11.501 | 1.00 | 40.02 |
| ATOM | 5408 | OD1 | ASN | B | 361 | 29.922 | 68.485 | 11.828 | 1.00 | 40.13 |
| ATOM | 5409 | ND2 | ASN | B | 361 | 30.414 | 70.171 | 10.402 | 1.00 | 41.04 |
| ATOM | 5410 | C | ASN | B | 361 | 30.330 | 71.154 | 14.458 | 1.00 | 33.34 |
| ATOM | 5411 | O | ASN | B | 361 | 31.319 | 71.430 | 13.774 | 1.00 | 31.51 |
| ATOM | 5412 | N | PHE | B | 362 | 30.196 | 71.521 | 15.743 | 1.00 | 31.80 |
| ATOM | 5413 | CA | PHE | B | 362 | 31.196 | 72.289 | 16.496 | 1.00 | 28.71 |
| ATOM | 5414 | CB | PHE | B | 362 | 30.653 | 72.660 | 17.886 | 1.00 | 26.16 |
| ATOM | 5415 | CG | PHE | B | 362 | 29.640 | 73.763 | 17.848 | 1.00 | 22.34 |
| ATOM | 5416 | CD1 | PHE | B | 362 | 28.290 | 73.482 | 17.918 | 1.00 | 22.56 |
| ATOM | 5417 | CD2 | PHE | B | 362 | 30.042 | 75.081 | 17.724 | 1.00 | 22.48 |
| ATOM | 5418 | CE1 | PHE | B | 362 | 27.356 | 74.496 | 17.867 | 1.00 | 22.49 |
| ATOM | 5419 | CE2 | PHE | B | 362 | 29.118 | 76.099 | 17.673 | 1.00 | 19.68 |
| ATOM | 5420 | CZ | PHE | B | 362 | 27.775 | 75.807 | 17.744 | 1.00 | 21.78 |
| ATOM | 5421 | C | PHE | B | 362 | 32.537 | 71.591 | 16.625 | 1.00 | 27.92 |
| ATOM | 5422 | O | PHE | B | 362 | 32.612 | 70.376 | 16.705 | 1.00 | 29.45 |
| ATOM | 5423 | N | THR | B | 363 | 33.601 | 72.388 | 16.658 | 1.00 | 29.26 |
| ATOM | 5424 | CA | THR | B | 363 | 34.954 | 71.859 | 16.783 | 1.00 | 30.32 |
| ATOM | 5425 | CB | THR | B | 363 | 35.778 | 72.296 | 15.582 | 1.00 | 29.40 |
| ATOM | 5426 | OG1 | THR | B | 363 | 37.077 | 71.754 | 15.666 | 1.00 | 32.75 |
| ATOM | 5427 | CG2 | THR | B | 363 | 35.928 | 73.795 | 15.485 | 1.00 | 29.08 |
| ATOM | 5428 | C | THR | B | 363 | 35.681 | 72.362 | 18.034 | 1.00 | 30.21 |
| ATOM | 5429 | O | THR | B | 363 | 35.297 | 73.357 | 18.643 | 1.00 | 28.68 |
| ATOM | 5430 | N | THR | B | 364 | 36.794 | 71.687 | 18.336 | 1.00 | 32.03 |
| ATOM | 5431 | CA | THR | B | 364 | 37.682 | 72.050 | 19.433 | 1.00 | 33.02 |
| ATOM | 5432 | CB | THR | B | 364 | 38.534 | 70.857 | 19.866 | 1.00 | 30.93 |
| ATOM | 5433 | OG1 | THR | B | 364 | 37.735 | 69.764 | 20.246 | 1.00 | 30.53 |
| ATOM | 5434 | CG2 | THR | B | 364 | 39.494 | 71.166 | 20.993 | 1.00 | 33.47 |
| ATOM | 5435 | C | THR | B | 364 | 38.596 | 73.166 | 18.942 | 1.00 | 34.09 |
| ATOM | 5436 | O | THR | B | 364 | 39.757 | 72.945 | 18.578 | 1.00 | 35.92 |
| ATOM | 5437 | N | GLN | B | 365 | 38.032 | 74.353 | 18.916 | 1.00 | 33.64 |
| ATOM | 5438 | CA | GLN | B | 365 | 38.682 | 75.578 | 18.468 | 1.00 | 31.88 |
| ATOM | 5439 | CB | GLN | B | 365 | 39.393 | 75.369 | 17.119 | 1.00 | 32.40 |
| ATOM | 5440 | CG | GLN | B | 365 | 39.723 | 76.653 | 16.360 | 1.00 | 30.97 |
| ATOM | 5441 | CD | GLN | B | 365 | 41.069 | 77.252 | 16.737 | 1.00 | 30.44 |
| ATOM | 5442 | OE1 | GLN | B | 365 | 41.166 | 78.439 | 17.028 | 1.00 | 31.75 |
| ATOM | 5443 | NE2 | GLN | B | 365 | 42.112 | 76.438 | 16.726 | 1.00 | 30.94 |
| ATOM | 5444 | C | GLN | B | 365 | 37.591 | 76.627 | 18.360 | 1.00 | 29.09 |
| ATOM | 5445 | O | GLN | B | 365 | 37.726 | 77.748 | 18.841 | 1.00 | 30.36 |
| ATOM | 5446 | N | GLU | B | 366 | 36.486 | 76.205 | 17.772 | 1.00 | 26.33 |
| ATOM | 5447 | CA | GLU | B | 366 | 35.306 | 77.026 | 17.614 | 1.00 | 26.16 |
| ATOM | 5448 | CB | GLU | B | 366 | 34.363 | 76.313 | 16.636 | 1.00 | 24.51 |
| ATOM | 5449 | CG | GLU | B | 366 | 33.272 | 77.179 | 16.026 | 1.00 | 22.75 |
| ATOM | 5450 | CD | GLU | B | 366 | 32.249 | 76.358 | 15.262 | 1.00 | 20.28 |
| ATOM | 5451 | OE1 | GLU | B | 366 | 31.130 | 76.851 | 15.057 | 1.00 | 21.44 |
| ATOM | 5452 | OE2 | GLU | B | 366 | 32.564 | 75.215 | 14.880 | 1.00 | 21.96 |
| ATOM | 5453 | C | GLU | B | 366 | 34.602 | 77.195 | 18.969 | 1.00 | 26.22 |
| ATOM | 5454 | O | GLU | B | 366 | 33.842 | 78.137 | 19.172 | 1.00 | 26.02 |
| ATOM | 5455 | N | LEU | B | 367 | 34.857 | 76.257 | 19.884 | 1.00 | 27.86 |
| ATOM | 5456 | CA | LEU | B | 367 | 34.251 | 76.269 | 21.217 | 1.00 | 28.45 |
| ATOM | 5457 | CB | LEU | B | 367 | 33.839 | 74.840 | 21.631 | 1.00 | 26.52 |
| ATOM | 5458 | CG | LEU | B | 367 | 32.727 | 74.155 | 20.818 | 1.00 | 24.71 |
| ATOM | 5459 | CD1 | LEU | B | 367 | 32.726 | 72.654 | 21.075 | 1.00 | 23.77 |
| ATOM | 5460 | CD2 | LEU | B | 367 | 31.362 | 74.745 | 21.135 | 1.00 | 24.85 |
| ATOM | 5461 | C | LEU | B | 367 | 35.209 | 76.829 | 22.272 | 1.00 | 29.25 |
| ATOM | 5462 | O | LEU | B | 367 | 34.798 | 77.109 | 23.393 | 1.00 | 28.89 |
| ATOM | 5463 | N | SER | B | 368 | 36.484 | 76.941 | 21.906 | 1.00 | 29.41 |
| ATOM | 5464 | CA | SER | B | 368 | 37.565 | 77.401 | 22.778 | 1.00 | 31.33 |
| ATOM | 5465 | CB | SER | B | 368 | 38.843 | 77.631 | 21.971 | 1.00 | 32.29 |
| ATOM | 5466 | OG | SER | B | 368 | 38.695 | 78.719 | 21.073 | 1.00 | 35.87 |
| ATOM | 5467 | C | SER | B | 368 | 37.260 | 78.613 | 23.675 | 1.00 | 32.52 |
| ATOM | 5468 | O | SER | B | 368 | 37.822 | 78.722 | 24.774 | 1.00 | 34.50 |
| ATOM | 5469 | N | SER | B | 369 | 36.418 | 79.531 | 23.219 | 1.00 | 31.73 |
| ATOM | 5470 | CA | SER | B | 369 | 36.107 | 80.729 | 24.006 | 1.00 | 30.54 |
| ATOM | 5471 | CB | SER | B | 369 | 35.446 | 81.806 | 23.140 | 1.00 | 27.93 |
| ATOM | 5472 | OG | SER | B | 369 | 34.055 | 81.585 | 23.014 | 1.00 | 26.02 |
| ATOM | 5473 | C | SER | B | 369 | 35.266 | 80.427 | 25.252 | 1.00 | 31.02 |
| ATOM | 5474 | O | SER | B | 369 | 35.196 | 81.246 | 26.169 | 1.00 | 32.02 |
| ATOM | 5475 | N | ASN | B | 370 | 34.632 | 79.256 | 25.268 | 1.00 | 30.60 |
| ATOM | 5476 | CA | ASN | B | 370 | 33.788 | 78.823 | 26.377 | 1.00 | 29.03 |
| ATOM | 5477 | CB | ASN | B | 370 | 32.537 | 79.707 | 26.453 | 1.00 | 30.95 |
| ATOM | 5478 | CG | ASN | B | 370 | 31.882 | 79.694 | 27.826 | 1.00 | 30.19 |
| ATOM | 5479 | OD1 | ASN | B | 370 | 31.168 | 80.623 | 28.197 | 1.00 | 30.56 |
| ATOM | 5480 | ND2 | ASN | B | 370 | 32.114 | 78.638 | 28.578 | 1.00 | 30.43 |
| ATOM | 5481 | C | ASN | B | 370 | 33.385 | 77.352 | 26.220 | 1.00 | 26.71 |
| ATOM | 5482 | O | ASN | B | 370 | 32.225 | 77.050 | 25.969 | 1.00 | 29.86 |
| ATOM | 5483 | N | PRO | B | 371 | 34.347 | 76.419 | 26.352 | 1.00 | 25.41 |
| ATOM | 5484 | CD | PRO | B | 371 | 35.765 | 76.702 | 26.633 | 1.00 | 25.72 |
| ATOM | 5485 | CA | PRO | B | 371 | 34.116 | 74.969 | 26.203 | 1.00 | 26.79 |
| ATOM | 5486 | CB | PRO | B | 371 | 35.322 | 74.361 | 26.903 | 1.00 | 26.64 |
| ATOM | 5487 | CG | PRO | B | 371 | 36.410 | 75.338 | 26.630 | 1.00 | 26.89 |
| ATOM | 5488 | C | PRO | B | 371 | 32.790 | 74.423 | 26.776 | 1.00 | 28.36 |
| ATOM | 5489 | O | PRO | B | 371 | 32.100 | 73.658 | 26.100 | 1.00 | 29.50 |
| ATOM | 5490 | N | PRO | B | 372 | 32.419 | 74.758 | 28.031 | 1.00 | 29.03 |
| ATOM | 5491 | CD | PRO | B | 372 | 33.178 | 75.609 | 28.965 | 1.00 | 29.71 |
| ATOM | 5492 | CA | PRO | B | 372 | 31.180 | 74.249 | 28.638 | 1.00 | 29.24 |
| ATOM | 5493 | CB | PRO | B | 372 | 31.158 | 74.870 | 30.042 | 1.00 | 29.18 |
| ATOM | 5494 | CG | PRO | B | 372 | 32.154 | 75.980 | 30.002 | 1.00 | 31.38 |
| ATOM | 5495 | C | PRO | B | 372 | 29.912 | 74.625 | 27.864 | 1.00 | 28.57 |
| ATOM | 5496 | O | PRO | B | 372 | 28.876 | 73.977 | 28.015 | 1.00 | 29.72 |
| ATOM | 5497 | N | LEU | B | 373 | 29.978 | 75.670 | 27.042 | 1.00 | 28.85 |
| ATOM | 5498 | CA | LEU | B | 373 | 28.798 | 76.087 | 26.276 | 1.00 | 28.73 |
| ATOM | 5499 | CB | LEU | B | 373 | 29.055 | 77.373 | 25.481 | 1.00 | 28.83 |
| ATOM | 5500 | CG | LEU | B | 373 | 28.932 | 78.681 | 26.272 | 1.00 | 29.03 |
| ATOM | 5501 | CD1 | LEU | B | 373 | 29.170 | 79.891 | 25.376 | 1.00 | 27.64 |
| ATOM | 5502 | CD2 | LEU | B | 373 | 27.577 | 78.781 | 26.959 | 1.00 | 29.15 |
| ATOM | 5503 | C | LEU | B | 373 | 28.266 | 74.957 | 25.387 | 1.00 | 27.06 |
| ATOM | 5504 | O | LEU | B | 373 | 27.118 | 74.986 | 24.956 | 1.00 | 27.47 |
| ATOM | 5505 | N | ALA | B | 374 | 29.099 | 73.946 | 25.161 | 1.00 | 27.71 |
| ATOM | 5506 | CA | ALA | B | 374 | 28.731 | 72.774 | 24.367 | 1.00 | 29.52 |
| ATOM | 5507 | CB | ALA | B | 374 | 29.912 | 71.828 | 24.238 | 1.00 | 28.86 |
| ATOM | 5508 | C | ALA | B | 374 | 27.520 | 72.029 | 24.936 | 1.00 | 30.78 |
| ATOM | 5509 | O | ALA | B | 374 | 26.806 | 71.353 | 24.190 | 1.00 | 30.95 |
| ATOM | 5510 | N | THR | B | 375 | 27.279 | 72.143 | 26.252 | 1.00 | 31.43 |
| ATOM | 5511 | CA | THR | B | 375 | 26.133 | 71.454 | 26.868 | 1.00 | 31.02 |
| ATOM | 5512 | CB | THR | B | 375 | 26.130 | 71.567 | 28.406 | 1.00 | 31.88 |
| ATOM | 5513 | OG1 | THR | B | 375 | 26.135 | 72.915 | 28.829 | 1.00 | 33.01 |
| ATOM | 5514 | CG2 | THR | B | 375 | 27.283 | 70.861 | 29.079 | 1.00 | 30.87 |
| ATOM | 5515 | C | THR | B | 375 | 24.809 | 71.933 | 26.288 | 1.00 | 29.69 |
| ATOM | 5516 | O | THR | B | 375 | 23.835 | 71.185 | 26.234 | 1.00 | 29.64 |
| ATOM | 5517 | N | ILE | B | 376 | 24.780 | 73.177 | 25.846 | 1.00 | 30.31 |
| ATOM | 5518 | CA | ILE | B | 376 | 23.577 | 73.754 | 25.254 | 1.00 | 30.34 |
| ATOM | 5519 | CB | ILE | B | 376 | 23.427 | 75.236 | 25.645 | 1.00 | 31.90 |
| ATOM | 5520 | CG2 | ILE | B | 376 | 22.130 | 75.811 | 25.093 | 1.00 | 33.49 |
| ATOM | 5521 | CG1 | ILE | B | 376 | 23.494 | 75.418 | 27.166 | 1.00 | 33.13 |
| ATOM | 5522 | CD1 | ILE | B | 376 | 23.394 | 76.865 | 27.603 | 1.00 | 30.17 |
| ATOM | 5523 | C | ILE | B | 376 | 23.648 | 73.672 | 23.720 | 1.00 | 29.48 |
| ATOM | 5524 | O | ILE | B | 376 | 22.706 | 73.231 | 23.063 | 1.00 | 28.38 |
| ATOM | 5525 | N | LEU | B | 377 | 24.780 | 74.123 | 23.182 | 1.00 | 29.90 |
| ATOM | 5526 | CA | LEU | B | 377 | 25.057 | 74.163 | 21.746 | 1.00 | 29.31 |
| ATOM | 5527 | CB | LEU | B | 377 | 26.473 | 74.667 | 21.509 | 1.00 | 27.56 |
| ATOM | 5528 | CG | LEU | B | 377 | 26.729 | 76.105 | 21.953 | 1.00 | 25.82 |
| ATOM | 5529 | CD1 | LEU | B | 377 | 28.185 | 76.483 | 21.739 | 1.00 | 24.88 |
| ATOM | 5530 | CD2 | LEU | B | 377 | 25.799 | 77.061 | 21.225 | 1.00 | 25.71 |
| ATOM | 5531 | C | LEU | B | 377 | 24.838 | 72.828 | 21.054 | 1.00 | 29.70 |
| ATOM | 5532 | O | LEU | B | 377 | 24.192 | 72.776 | 20.019 | 1.00 | 30.42 |
| ATOM | 5533 | N | ILE | B | 378 | 25.363 | 71.754 | 21.643 | 1.00 | 30.75 |
| ATOM | 5534 | CA | ILE | B | 378 | 25.211 | 70.413 | 21.097 | 1.00 | 30.50 |
| ATOM | 5535 | CB | ILE | B | 378 | 26.559 | 69.669 | 21.074 | 1.00 | 30.22 |
| ATOM | 5536 | CG2 | ILE | B | 378 | 26.400 | 68.295 | 20.428 | 1.00 | 30.35 |
| ATOM | 5537 | CG1 | ILE | B | 378 | 27.607 | 70.511 | 20.323 | 1.00 | 28.02 |
| ATOM | 5538 | CD1 | ILE | B | 378 | 29.016 | 69.974 | 20.416 | 1.00 | 27.08 |
| ATOM | 5539 | C | ILE | B | 378 | 24.173 | 69.605 | 21.882 | 1.00 | 32.15 |
| ATOM | 5540 | O | ILE | B | 378 | 24.491 | 68.998 | 22.905 | 1.00 | 33.58 |
| ATOM | 5541 | N | PRO | B | 379 | 22.908 | 69.598 | 21.418 | 1.00 | 32.79 |
| ATOM | 5542 | CD | PRO | B | 379 | 22.434 | 70.307 | 20.215 | 1.00 | 34.93 |
| ATOM | 5543 | CA | PRO | B | 379 | 21.819 | 68.875 | 22.084 | 1.00 | 33.56 |
| ATOM | 5544 | CB | PRO | B | 379 | 20.581 | 69.214 | 21.244 | 1.00 | 33.64 |
| ATOM | 5545 | CG | PRO | B | 379 | 21.127 | 69.633 | 19.922 | 1.00 | 34.99 |
| ATOM | 5546 | C | PRO | B | 379 | 22.053 | 67.369 | 22.126 | 1.00 | 33.49 |
| ATOM | 5547 | O | PRO | B | 379 | 22.885 | 66.844 | 21.385 | 1.00 | 34.07 |
| ATOM | 5548 | N | PRO | B | 380 | 21.328 | 66.659 | 23.020 | 1.00 | 33.74 |
| ATOM | 5549 | CD | PRO | B | 380 | 20.333 | 67.233 | 23.959 | 1.00 | 34.16 |
| ATOM | 5550 | CA | PRO | B | 380 | 21.458 | 65.197 | 23.196 | 1.00 | 33.82 |
| ATOM | 5551 | CB | PRO | B | 380 | 20.300 | 64.850 | 24.139 | 1.00 | 33.23 |
| ATOM | 5552 | CG | PRO | B | 380 | 20.072 | 66.100 | 24.917 | 1.00 | 34.15 |
| ATOM | 5553 | C | PRO | B | 380 | 21.338 | 64.405 | 21.890 | 1.00 | 33.59 |
| ATOM | 5554 | O | PRO | B | 380 | 22.153 | 63.528 | 21.617 | 1.00 | 33.92 |
| ATOM | 5555 | N | HIS | B | 381 | 20.320 | 64.713 | 21.096 | 1.00 | 35.19 |
| ATOM | 5556 | CA | HIS | B | 381 | 20.081 | 64.027 | 19.825 | 1.00 | 37.19 |
| ATOM | 5557 | CB | HIS | B | 381 | 18.759 | 64.499 | 19.210 | 1.00 | 38.15 |
| ATOM | 5558 | CG | HIS | B | 381 | 18.812 | 65.874 | 18.604 | 1.00 | 38.93 |
| ATOM | 5559 | CD2 | HIS | B | 381 | 18.392 | 67.070 | 19.077 | 1.00 | 39.05 |
| ATOM | 5560 | ND1 | HIS | B | 381 | 19.341 | 66.129 | 17.353 | 1.00 | 39.55 |
| ATOM | 5561 | CE1 | HIS | B | 381 | 19.246 | 67.420 | 17.084 | 1.00 | 38.69 |
| ATOM | 5562 | NE2 | HIS | B | 381 | 18.673 | 68.013 | 18.115 | 1.00 | 38.31 |
| ATOM | 5563 | C | HIS | B | 381 | 21.221 | 64.215 | 18.817 | 1.00 | 39.12 |
| ATOM | 5564 | O | HIS | B | 381 | 21.357 | 63.427 | 17.876 | 1.00 | 39.80 |
| ATOM | 5565 | N | ALA | B | 382 | 22.013 | 65.273 | 19.002 | 1.00 | 40.26 |
| ATOM | 5566 | CA | ALA | B | 382 | 23.119 | 65.594 | 18.101 | 1.00 | 41.09 |
| ATOM | 5567 | CB | ALA | B | 382 | 23.175 | 67.097 | 17.866 | 1.00 | 40.37 |
| ATOM | 5568 | C | ALA | B | 382 | 24.473 | 65.093 | 18.596 | 1.00 | 42.29 |
| ATOM | 5569 | O | ALA | B | 382 | 25.485 | 65.306 | 17.935 | 1.00 | 41.80 |
| ATOM | 5570 | N | ARG | B | 383 | 24.504 | 64.439 | 19.752 | 1.00 | 43.81 |
| ATOM | 5571 | CA | ARG | B | 383 | 25.757 | 63.940 | 20.299 | 1.00 | 46.02 |
| ATOM | 5572 | CB | ARG | B | 383 | 25.711 | 63.948 | 21.831 | 1.00 | 44.99 |
| ATOM | 5573 | CG | ARG | B | 383 | 25.911 | 65.324 | 22.441 | 1.00 | 42.60 |
| ATOM | 5574 | CD | ARG | B | 383 | 25.629 | 65.324 | 23.937 | 1.00 | 41.32 |
| ATOM | 5575 | NE | ARG | B | 383 | 25.029 | 66.586 | 24.376 | 1.00 | 40.35 |
| ATOM | 5576 | CZ | ARG | B | 383 | 24.259 | 66.720 | 25.458 | 1.00 | 38.67 |
| ATOM | 5577 | NH1 | ARG | B | 383 | 24.014 | 65.677 | 26.250 | 1.00 | 37.27 |
| ATOM | 5578 | NH2 | ARG | B | 383 | 23.741 | 67.908 | 25.751 | 1.00 | 36.51 |
| ATOM | 5579 | C | ARG | B | 383 | 26.062 | 62.533 | 19.788 | 1.00 | 48.67 |
| ATOM | 5580 | O | ARG | B | 383 | 25.150 | 61.745 | 19.516 | 1.00 | 49.21 |
| ATOM | 5581 | N | ILE | B | 384 | 27.355 | 62.223 | 19.671 | 1.00 | 51.03 |
| ATOM | 5582 | CA | ILE | B | 384 | 27.798 | 60.910 | 19.205 | 1.00 | 52.96 |
| ATOM | 5583 | CB | ILE | B | 384 | 28.920 | 61.013 | 18.148 | 1.00 | 52.76 |
| ATOM | 5584 | CG2 | ILE | B | 384 | 29.157 | 59.658 | 17.496 | 1.00 | 52.75 |
| ATOM | 5585 | CG1 | ILE | B | 384 | 28.563 | 62.059 | 17.086 | 1.00 | 51.93 |
| ATOM | 5586 | CD1 | ILE | B | 384 | 29.492 | 63.253 | 17.068 | 1.00 | 51.77 |
| ATOM | 5587 | C | ILE | B | 384 | 28.272 | 60.041 | 20.372 | 1.00 | 54.86 |
| ATOM | 5588 | OT1 | ILE | B | 384 | 28.001 | 58.815 | 20.344 | 1.00 | 55.31 |
| ATOM | 5589 | OXT | ILE | B | 384 | 28.909 | 60.593 | 21.309 | 1.00 | 55.89 |
| ATOM | 5590 | | ILE | B | 384 | | | | | |
| ATOM | 5591 | OH2 | TIP | | 1 | 20.184 | 97.209 | 9.978 | 1.00 | 14.30 |
| ATOM | 5592 | OH2 | TIP | | 2 | 86.592 | 49.067 | 10.183 | 1.00 | 15.43 |
| ATOM | 5593 | OH2 | TIP | | 3 | 17.055 | 66.016 | 22.246 | 1.00 | 16.08 |
| ATOM | 5594 | OH2 | TIP | | 4 | 43.676 | 99.232 | 10.734 | 1.00 | 18.27 |
| ATOM | 5595 | OH2 | TIP | | 5 | 74.057 | 43.510 | 23.648 | 1.00 | 8.65 |
| ATOM | 5596 | OH2 | TIP | | 6 | 79.150 | 41.199 | 7.662 | 1.00 | 16.64 |
| ATOM | 5597 | OH2 | TIP | | 7 | 73.604 | 48.334 | 14.257 | 1.00 | 17.10 |
| ATOM | 5598 | OH2 | TIP | | 8 | 71.710 | 51.582 | 3.800 | 1.00 | 26.80 |
| ATOM | 5599 | OH2 | TIP | | 9 | 74.647 | 44.648 | 12.301 | 1.00 | 25.74 |
| ATOM | 5600 | OH2 | TIP | | 10 | 68.184 | 50.139 | 5.114 | 1.00 | 22.46 |
| ATOM | 5601 | OH2 | TIP | | 11 | 30.995 | 97.272 | 13.532 | 1.00 | 24.09 |
| ATOM | 5602 | OH2 | TIP | | 12 | 31.473 | 94.065 | 11.111 | 1.00 | 35.85 |
| ATOM | 5603 | OH2 | TIP | | 13 | 88.665 | 47.357 | 16.164 | 1.00 | 21.52 |
| ATOM | 5604 | OH2 | TIP | | 14 | 62.931 | 47.153 | 11.083 | 1.00 | 15.13 |
| ATOM | 5605 | OH2 | TIP | | 15 | 32.589 | 83.518 | 20.788 | 1.00 | 21.19 |
| ATOM | 5606 | OH2 | TIP | | 16 | 76.928 | 36.781 | 1.256 | 1.00 | 35.32 |
| ATOM | 5607 | OH2 | TIP | | 17 | 77.542 | 40.357 | 22.692 | 1.00 | 20.20 |
| ATOM | 5608 | OH2 | TIP | | 18 | 39.998 | 74.585 | 11.864 | 1.00 | 33.26 |
| ATOM | 5609 | OH2 | TIP | | 19 | 75.908 | 25.776 | 8.313 | 1.00 | 31.07 |
| ATOM | 5610 | OH2 | TIP | | 20 | 36.308 | 91.732 | 5.629 | 1.00 | 20.75 |
| ATOM | 5611 | OH2 | TIP | | 21 | 34.229 | 87.485 | 20.266 | 1.00 | 30.78 |
| ATOM | 5612 | OH2 | TIP | | 22 | 69.815 | 37.826 | 0.045 | 1.00 | 31.95 |
| ATOM | 5613 | OH2 | TIP | | 23 | 78.149 | 47.463 | 24.796 | 1.00 | 15.46 |
| ATOM | 5614 | OH2 | TIP | | 24 | 67.941 | 51.729 | 13.432 | 1.00 | 17.40 |
| ATOM | 5615 | OH2 | TIP | | 25 | 69.556 | 26.809 | 16.650 | 1.00 | 25.99 |
| ATOM | 5616 | OH2 | TIP | | 26 | 82.058 | 56.569 | 8.529 | 1.00 | 32.89 |
| ATOM | 5617 | OH2 | TIP | | 27 | 34.979 | 82.693 | 19.866 | 1.00 | 23.31 |
| ATOM | 5618 | OH2 | TIP | | 28 | 23.280 | 100.047 | 25.484 | 1.00 | 24.88 |
| ATOM | 5619 | OH2 | TIP | | 29 | 35.288 | 97.692 | 5.905 | 1.00 | 22.00 |
| ATOM | 5620 | OH2 | TIP | | 30 | 76.241 | 56.395 | 10.831 | 1.00 | 16.49 |
| ATOM | 5621 | OH2 | TIP | | 31 | 31.026 | 90.281 | 21.603 | 1.00 | 13.40 |
| ATOM | 5622 | OH2 | TIP | | 32 | 16.272 | 85.213 | 27.168 | 1.00 | 31.73 |
| ATOM | 5623 | OH2 | TIP | | 33 | 34.623 | 80.209 | 20.638 | 1.00 | 22.53 |
| ATOM | 5624 | OH2 | TIP | | 34 | 23.509 | 83.346 | 25.930 | 1.00 | 26.25 |
| ATOM | 5625 | OH2 | TIP | | 35 | 68.471 | 35.753 | −6.941 | 1.00 | 31.74 |
| ATOM | 5626 | OH2 | TIP | | 36 | 42.708 | 91.595 | −1.200 | 1.00 | 30.13 |
| ATOM | 5627 | OH2 | TIP | | 37 | 73.882 | 57.001 | 9.723 | 1.00 | 30.46 |
| ATOM | 5628 | OH2 | TIP | | 38 | 19.201 | 109.504 | 8.536 | 1.00 | 27.81 |
| ATOM | 5629 | OH2 | TIP | | 39 | 66.027 | 53.748 | 25.075 | 1.00 | 23.62 |
| ATOM | 5630 | OH2 | TIP | | 40 | 32.828 | 76.455 | 3.143 | 1.00 | 18.78 |
| ATOM | 5631 | OH2 | TIP | | 41 | 31.297 | 75.533 | −5.044 | 1.00 | 34.74 |
| ATOM | 5632 | OH2 | TIP | | 42 | 46.752 | 86.115 | 9.500 | 1.00 | 41.15 |
| ATOM | 5633 | OH2 | TIP | | 43 | 45.965 | 90.475 | 9.941 | 1.00 | 19.78 |
| ATOM | 5634 | OH2 | TIP | | 44 | 84.824 | 40.900 | 6.762 | 1.00 | 26.00 |
| ATOM | 5635 | OH2 | TIP | | 45 | 88.709 | 40.949 | 11.546 | 1.00 | 24.12 |
| ATOM | 5636 | OH2 | TIP | | 46 | 33.051 | 73.353 | 10.148 | 1.00 | 34.00 |
| ATOM | 5637 | OH2 | TIP | | 47 | 11.488 | 88.500 | 10.474 | 1.00 | 25.91 |
| ATOM | 5638 | OH2 | TIP | | 48 | 35.424 | 94.037 | 2.827 | 1.00 | 22.82 |
| ATOM | 5639 | OH2 | TIP | | 49 | 73.417 | 40.151 | 1.138 | 1.00 | 33.97 |
| ATOM | 5640 | OH2 | TIP | | 50 | 64.215 | 49.505 | 11.135 | 1.00 | 29.96 |
| ATOM | 5641 | OH2 | TIP | | 51 | 35.946 | 77.916 | 5.004 | 1.00 | 22.91 |
| ATOM | 5642 | OH2 | TIP | | 52 | 19.095 | 111.400 | 18.136 | 1.00 | 32.18 |
| ATOM | 5643 | OH2 | TIP | | 53 | 87.512 | 32.734 | 22.035 | 1.00 | 30.17 |
| ATOM | 5644 | OH2 | TIP | | 54 | 77.551 | 47.032 | 6.411 | 1.00 | 26.58 |
| ATOM | 5645 | OH2 | TIP | | 55 | 86.637 | 41.109 | 3.619 | 1.00 | 26.63 |
| ATOM | 5646 | OH2 | TIP | | 56 | 58.706 | 37.981 | −1.600 | 1.00 | 33.07 |
| ATOM | 5647 | OH2 | TIP | | 57 | 44.991 | 91.904 | 1.544 | 1.00 | 28.58 |
| ATOM | 5648 | OH2 | TIP | | 58 | 82.871 | 58.902 | 12.437 | 1.00 | 29.62 |
| ATOM | 5649 | OH2 | TIP | | 59 | 62.486 | 37.207 | 30.060 | 1.00 | 27.58 |
| ATOM | 5650 | OH2 | TIP | | 60 | 32.219 | 106.679 | 11.372 | 1.00 | 35.07 |
| ATOM | 5651 | OH2 | TIP | | 61 | 68.981 | 58.634 | 30.748 | 1.00 | 27.70 |
| ATOM | 5652 | OH2 | TIP | | 62 | 80.283 | 44.331 | −8.715 | 1.00 | 20.83 |
| ATOM | 5653 | OH2 | TIP | | 63 | 25.093 | 71.081 | 5.643 | 1.00 | 31.03 |
| ATOM | 5654 | OH2 | TIP | | 64 | 38.840 | 87.644 | 18.843 | 1.00 | 32.58 |
| ATOM | 5655 | OH2 | TIP | | 65 | 35.643 | 97.747 | −3.291 | 1.00 | 33.06 |
| ATOM | 5656 | OH2 | TIP | | 66 | 28.484 | 90.260 | 29.530 | 1.00 | 38.62 |
| ATOM | 5657 | OH2 | TIP | | 67 | 32.057 | 83.314 | 26.983 | 1.00 | 26.10 |
| ATOM | 5658 | OH2 | TIP | | 68 | 40.728 | 81.620 | 21.277 | 1.00 | 28.96 |
| ATOM | 5659 | OH2 | TIP | | 69 | 83.110 | 48.185 | −6.280 | 1.00 | 36.25 |
| ATOM | 5660 | OH2 | TIP | | 70 | 13.308 | 79.186 | 19.324 | 1.00 | 27.35 |
| ATOM | 5661 | OH2 | TIP | | 71 | 75.121 | 65.908 | 23.710 | 1.00 | 33.33 |
| ATOM | 5662 | OH2 | TIP | | 72 | 72.885 | 49.529 | 31.358 | 1.00 | 43.43 |
| ATOM | 5663 | OH2 | TIP | | 73 | 90.409 | 43.863 | 22.223 | 1.00 | 35.52 |
| ATOM | 5664 | OH2 | TIP | | 74 | 68.960 | 27.956 | 6.246 | 1.00 | 29.54 |
| ATOM | 5665 | OH2 | TIP | | 75 | 68.759 | 24.978 | 13.691 | 1.00 | 36.46 |
| ATOM | 5666 | OH2 | TIP | | 76 | 80.865 | 28.042 | 28.894 | 1.00 | 22.81 |
| ATOM | 5667 | OH2 | TIP | | 77 | 83.513 | 20.534 | 2.136 | 1.00 | 44.57 |
| ATOM | 5668 | OH2 | TIP | | 78 | 33.991 | 88.631 | −1.759 | 1.00 | 47.18 |
| ATOM | 5669 | OH2 | TIP | | 79 | 31.009 | 90.742 | −1.589 | 1.00 | 35.71 |
| ATOM | 5670 | OH2 | TIP | | 80 | 34.056 | 73.695 | 12.888 | 1.00 | 40.61 |
| ATOM | 5671 | OH2 | TIP | | 81 | 71.030 | 59.790 | 1.571 | 1.00 | 34.14 |
| ATOM | 5672 | OH2 | TIP | | 82 | 45.548 | 95.239 | 15.831 | 1.00 | 31.62 |
| ATOM | 5673 | OH2 | TIP | | 83 | 25.823 | 76.105 | 11.243 | 1.00 | 25.69 |
| ATOM | 5674 | OH2 | TIP | | 84 | 75.453 | 38.146 | 30.507 | 1.00 | 25.25 |
| ATOM | 5675 | OH2 | TIP | | 85 | 56.540 | 32.057 | 24.557 | 1.00 | 40.19 |
| ATOM | 5676 | OH2 | TIP | | 86 | 68.921 | 53.407 | 27.143 | 1.00 | 32.24 |
| ATOM | 5677 | OH2 | TIP | | 87 | 48.823 | 99.292 | 13.844 | 1.00 | 48.14 |
| ATOM | 5678 | OH2 | TIP | | 88 | 66.603 | 27.914 | 24.871 | 1.00 | 24.07 |
| ATOM | 5679 | OH2 | TIP | | 89 | 72.553 | 25.024 | 4.480 | 1.00 | 37.79 |
| ATOM | 5680 | OH2 | TIP | | 90 | 74.805 | 44.624 | 26.080 | 1.00 | 24.08 |
| ATOM | 5681 | OH2 | TIP | | 91 | 82.314 | 60.516 | 10.339 | 1.00 | 28.44 |
| ATOM | 5682 | OH2 | TIP | | 92 | 41.009 | 72.787 | −2.680 | 1.00 | 49.99 |
| ATOM | 5683 | OH2 | TIP | | 93 | 75.218 | 36.521 | 28.025 | 1.00 | 21.73 |
| ATOM | 5684 | OH2 | TIP | | 94 | 58.786 | 33.514 | −1.584 | 1.00 | 46.12 |
| ATOM | 5685 | OH2 | TIP | | 95 | 59.443 | 56.186 | 13.092 | 1.00 | 24.57 |
| ATOM | 5686 | OH2 | TIP | | 96 | 44.571 | 79.740 | 17.158 | 1.00 | 33.72 |
| ATOM | 5687 | OH2 | TIP | | 97 | 36.543 | 97.946 | 22.228 | 1.00 | 20.11 |
| ATOM | 5688 | OH2 | TIP | | 98 | 42.102 | 91.284 | 4.898 | 1.00 | 22.27 |
| ATOM | 5689 | OH2 | TIP | | 99 | 83.993 | 26.068 | 16.812 | 1.00 | 39.48 |
| ATOM | 5690 | OH2 | TIP | | 100 | 71.425 | 22.395 | 31.294 | 1.00 | 42.78 |
| ATOM | 5691 | OH2 | TIP | | 101 | 41.521 | 86.237 | 20.453 | 1.00 | 36.59 |
| ATOM | 5692 | OH2 | TIP | | 102 | 77.290 | 21.465 | 18.797 | 1.00 | 39.54 |
| ATOM | 5693 | OH2 | TIP | | 103 | 34.574 | 71.613 | 8.514 | 1.00 | 40.98 |
| ATOM | 5694 | OH2 | TIP | | 104 | 80.216 | 58.145 | 30.951 | 1.00 | 31.65 |
| ATOM | 5695 | OH2 | TIP | | 105 | 51.953 | 83.113 | −1.215 | 1.00 | 34.53 |
| ATOM | 5696 | OH2 | TIP | | 106 | 78.065 | 52.209 | 26.693 | 1.00 | 31.02 |
| ATOM | 5697 | OH2 | TIP | | 107 | 68.221 | 20.544 | 13.056 | 1.00 | 29.84 |
| ATOM | 5698 | OH2 | TIP | | 108 | 9.340 | 73.178 | −1.320 | 1.00 | 27.34 |
| ATOM | 5699 | OH2 | TIP | | 109 | 82.865 | 28.096 | 7.563 | 1.00 | 24.08 |
| ATOM | 5700 | OH2 | TIP | | 110 | 51.681 | 22.615 | 23.853 | 1.00 | 42.57 |
| ATOM | 5701 | OH2 | TIP | | 111 | 80.577 | 21.841 | 24.237 | 1.00 | 38.89 |
| ATOM | 5702 | OH2 | TIP | | 112 | 29.043 | 102.944 | 3.861 | 1.00 | 36.35 |
| ATOM | 5703 | OH2 | TIP | | 113 | 34.227 | 97.988 | 29.010 | 1.00 | 29.76 |
| ATOM | 5704 | OH2 | TIP | | 114 | 40.735 | 90.064 | 18.442 | 1.00 | 46.42 |
| ATOM | 5705 | OH2 | TIP | | 115 | 25.295 | 100.974 | 13.456 | 1.00 | 25.31 |
| ATOM | 5706 | OH2 | TIP | | 116 | 75.441 | 52.065 | 2.449 | 1.00 | 19.42 |
| ATOM | 5707 | OH2 | TIP | | 117 | 74.070 | 56.553 | −0.631 | 1.00 | 47.18 |
| ATOM | 5708 | OH2 | TIP | | 118 | 65.566 | 64.590 | 9.062 | 1.00 | 44.16 |
| ATOM | 5709 | OH2 | TIP | | 119 | 60.666 | 43.201 | 3.854 | 1.00 | 34.57 |
| ATOM | 5710 | OH2 | TIP | | 120 | 76.120 | 45.123 | 29.617 | 1.00 | 36.08 |
| ATOM | 5711 | OH2 | TIP | | 121 | 54.142 | 51.739 | 14.688 | 1.00 | 31.52 |
| ATOM | 5712 | OH2 | TIP | | 122 | 39.719 | 105.971 | 10.102 | 1.00 | 27.01 |
| ATOM | 5713 | OH2 | TIP | | 123 | 85.171 | 44.498 | 7.479 | 1.00 | 31.29 |
| ATOM | 5714 | OH2 | TIP | | 124 | 86.210 | 29.970 | −11.184 | 1.00 | 29.65 |
| ATOM | 5715 | OH2 | TIP | | 125 | 72.634 | 31.654 | −0.249 | 1.00 | 25.24 |
| ATOM | 5716 | OH2 | TIP | | 126 | 16.147 | 70.198 | 22.206 | 1.00 | 28.88 |
| ATOM | 5717 | OH2 | TIP | | 127 | 37.231 | 69.031 | 17.117 | 1.00 | 39.61 |
| ATOM | 5718 | OH2 | TIP | | 128 | 35.202 | 98.368 | 26.457 | 1.00 | 23.25 |
| ATOM | 5719 | OH2 | TIP | | 129 | 101.610 | 39.762 | −20.072 | 1.00 | 30.45 |
| ATOM | 5720 | OH2 | TIP | | 130 | 62.699 | 33.014 | −2.726 | 1.00 | 42.68 |
| ATOM | 5721 | OH2 | TIP | | 131 | 66.836 | 50.243 | 30.662 | 1.00 | 39.68 |
| ATOM | 5722 | OH2 | TIP | | 132 | 5.358 | 93.633 | 5.849 | 1.00 | 48.29 |
| ATOM | 5723 | OH2 | TIP | | 133 | 87.786 | 24.130 | −16.104 | 1.00 | 39.99 |
| ATOM | 5724 | OH2 | TIP | | 134 | 42.301 | 75.221 | 8.757 | 1.00 | 42.10 |
| ATOM | 5725 | OH2 | TIP | | 135 | 78.036 | 44.723 | −10.662 | 1.00 | 37.65 |
| ATOM | 5726 | OH2 | TIP | | 136 | 38.073 | 90.012 | 21.232 | 1.00 | 23.10 |
| ATOM | 5727 | OH2 | TIP | | 137 | 24.581 | 92.415 | −2.442 | 1.00 | 37.05 |
| ATOM | 5728 | OH2 | TIP | | 138 | 12.396 | 106.041 | 18.053 | 1.00 | 43.25 |
| ATOM | 5729 | OH2 | TIP | | 139 | 77.977 | 34.593 | 3.036 | 1.00 | 25.44 |
| ATOM | 5730 | OH2 | TIP | | 140 | 40.297 | 92.891 | −1.662 | 1.00 | 40.79 |
| ATOM | 5731 | OH2 | TIP | | 141 | 35.031 | 85.843 | −0.290 | 1.00 | 30.75 |
| ATOM | 5732 | OH2 | TIP | | 142 | 78.177 | 49.267 | 4.406 | 1.00 | 44.08 |
| ATOM | 5733 | OH2 | TIP | | 143 | 44.684 | 85.363 | −13.653 | 1.00 | 42.96 |
| ATOM | 5734 | OH2 | TIP | | 144 | 16.523 | 68.716 | 10.379 | 1.00 | 44.38 |
| ATOM | 5735 | OH2 | TIP | | 145 | 37.171 | 84.228 | 20.730 | 1.00 | 37.73 |
| ATOM | 5736 | OH2 | TIP | | 146 | 84.109 | 34.734 | 25.222 | 1.00 | 28.75 |
| ATOM | 5737 | OH2 | TIP | | 147 | 13.573 | 65.829 | 5.292 | 1.00 | 41.32 |
| ATOM | 5738 | OH2 | TIP | | 148 | 87.775 | 51.477 | 19.308 | 1.00 | 33.04 |
| ATOM | 5739 | OH2 | TIP | | 149 | 61.055 | 46.851 | 29.860 | 1.00 | 38.86 |
| ATOM | 5740 | OH2 | TIP | | 150 | 30.709 | 93.775 | 30.547 | 1.00 | 35.96 |
| ATOM | 5741 | OH2 | TIP | | 151 | 16.687 | 95.444 | 4.127 | 1.00 | 34.28 |
| ATOM | 5742 | OH2 | TIP | | 152 | 20.727 | 95.654 | 3.238 | 1.00 | 38.20 |
| ATOM | 5743 | OH2 | TIP | | 153 | 77.522 | 26.578 | −16.506 | 1.00 | 34.79 |
| ATOM | 5744 | OH2 | TIP | | 154 | 17.542 | 77.986 | 26.146 | 1.00 | 40.07 |
| ATOM | 5745 | OH2 | TIP | | 155 | 63.161 | 38.672 | −5.820 | 1.00 | 54.97 |
| ATOM | 5746 | OH2 | TIP | | 156 | 74.192 | 34.524 | 35.857 | 1.00 | 57.51 |
| ATOM | 5747 | OH2 | TIP | | 157 | 21.552 | 99.456 | 10.491 | 1.00 | 27.88 |
| ATOM | 5748 | OH2 | TIP | | 158 | 83.085 | 24.564 | 22.892 | 1.00 | 31.49 |
| ATOM | 5749 | OH2 | TIP | | 159 | 47.798 | 39.263 | −2.076 | 1.00 | 54.69 |
| ATOM | 5750 | OH2 | TIP | | 160 | 88.099 | 51.183 | 23.984 | 1.00 | 35.12 |
| ATOM | 5751 | OH2 | TIP | | 161 | 24.423 | 92.769 | 30.535 | 1.00 | 38.45 |
| ATOM | 5752 | OH2 | TIP | | 162 | 63.166 | 20.190 | 11.461 | 1.00 | 46.78 |
| ATOM | 5753 | OH2 | TIP | | 163 | 59.098 | 44.329 | 5.827 | 1.00 | 33.01 |
| ATOM | 5754 | OH2 | TIP | | 164 | 13.507 | 107.912 | 20.781 | 1.00 | 35.72 |
| ATOM | 5755 | OH2 | TIP | | 165 | 83.412 | 61.086 | 22.636 | 1.00 | 35.01 |
| ATOM | 5756 | OH2 | TIP | | 166 | 49.112 | 73.913 | 6.534 | 1.00 | 66.27 |
| ATOM | 5757 | OH2 | TIP | | 167 | 91.394 | 32.212 | 10.337 | 1.00 | 50.39 |
| ATOM | 5758 | OH2 | TIP | | 168 | 6.449 | 94.287 | 26.888 | 1.00 | 47.63 |
| ATOM | 5759 | OH2 | TIP | | 169 | 76.464 | 68.219 | 23.721 | 1.00 | 53.79 |
| ATOM | 5760 | OH2 | TIP | | 170 | 22.808 | 74.862 | 19.059 | 1.00 | 33.21 |
| ATOM | 5761 | OH2 | TIP | | 171 | 48.317 | 80.149 | 7.342 | 1.00 | 27.26 |
| ATOM | 5762 | OH2 | TIP | | 172 | 41.162 | 85.237 | −16.033 | 1.00 | 39.98 |
| ATOM | 5763 | OH2 | TIP | | 173 | 51.407 | 31.394 | 15.649 | 1.00 | 42.59 |
| ATOM | 5764 | OH2 | TIP | | 174 | 22.955 | 60.844 | 22.421 | 1.00 | 38.82 |
| ATOM | 5765 | OH2 | TIP | | 175 | 28.394 | 98.779 | 1.827 | 1.00 | 41.53 |
| ATOM | 5766 | OH2 | TIP | | 176 | 69.684 | 65.594 | 24.524 | 1.00 | 44.96 |
| ATOM | 5767 | OH2 | TIP | | 177 | 43.200 | 93.280 | 3.459 | 1.00 | 34.13 |
| ATOM | 5768 | OH2 | TIP | | 178 | 17.756 | 90.119 | 27.355 | 1.00 | 45.97 |
| ATOM | 5769 | OH2 | TIP | | 179 | 105.475 | 35.722 | 3.208 | 1.00 | 46.41 |
| ATOM | 5770 | OH2 | TIP | | 180 | 18.511 | 69.070 | 13.131 | 1.00 | 44.83 |
| ATOM | 5771 | OH2 | TIP | | 181 | 34.777 | 83.786 | 27.054 | 1.00 | 49.09 |
| ATOM | 5772 | OH2 | TIP | | 182 | 76.901 | 25.642 | 31.034 | 1.00 | 47.31 |
| ATOM | 5773 | OH2 | TIP | | 183 | 48.876 | 35.979 | 8.482 | 1.00 | 32.37 |
| ATOM | 5774 | OH2 | TIP | | 184 | 25.637 | 99.645 | 26.739 | 1.00 | 45.36 |
| ATOM | 5775 | OH2 | TIP | | 185 | 93.103 | 36.239 | 0.414 | 1.00 | 33.77 |
| ATOM | 5776 | OH2 | TIP | | 186 | 71.600 | 55.767 | 30.268 | 1.00 | 32.79 |
| ATOM | 5777 | OH2 | TIP | | 187 | 15.541 | 76.607 | 2.456 | 1.00 | 41.98 |
| ATOM | 5778 | OH2 | TIP | | 188 | 41.719 | 74.503 | 14.650 | 1.00 | 51.88 |
| ATOM | 5779 | OH2 | TIP | | 189 | 75.164 | 69.545 | 21.068 | 1.00 | 52.07 |
| ATOM | 5780 | OH2 | TIP | | 190 | 74.872 | 39.797 | −2.637 | 1.00 | 41.24 |
| ATOM | 5781 | OH2 | TIP | | 191 | 22.684 | 97.520 | 3.087 | 1.00 | 37.30 |
| ATOM | 5782 | OH2 | TIP | | 192 | 25.467 | 107.654 | 1.749 | 1.00 | 41.11 |
| ATOM | 5783 | OH2 | TIP | | 193 | 69.282 | 51.934 | 2.697 | 1.00 | 41.69 |
| ATOM | 5784 | OH2 | TIP | | 194 | 38.994 | 107.268 | 27.347 | 1.00 | 37.35 |
| ATOM | 5785 | OH2 | TIP | | 195 | 63.092 | 51.837 | 28.741 | 1.00 | 38.92 |
| ATOM | 5786 | OH2 | TIP | | 196 | 11.487 | 77.282 | 0.147 | 1.00 | 36.21 |
| ATOM | 5787 | OH2 | TIP | | 197 | 36.150 | 94.786 | −15.531 | 1.00 | 39.60 |
| ATOM | 5788 | OH2 | TIP | | 198 | 82.360 | 44.024 | 21.173 | 1.00 | 36.55 |
| ATOM | 5789 | OH2 | TIP | | 199 | 35.092 | 94.097 | 23.903 | 1.00 | 32.06 |
| ATOM | 5790 | OH2 | TIP | | 200 | 63.021 | 70.590 | 15.903 | 1.00 | 56.60 |
| ATOM | 5791 | OH2 | TIP | | 201 | 49.283 | 43.418 | 9.849 | 1.00 | 47.58 |
| ATOM | 5792 | OH2 | TIP | | 202 | 36.906 | 100.577 | 21.840 | 1.00 | 40.78 |
| ATOM | 5793 | OH2 | TIP | | 203 | 76.551 | 63.953 | 25.972 | 1.00 | 35.46 |
| ATOM | 5794 | OH2 | TIP | | 204 | 87.842 | 55.120 | 11.824 | 1.00 | 29.94 |
| ATOM | 5795 | OH2 | TIP | | 205 | 36.937 | 66.014 | −8.244 | 1.00 | 41.61 |
| ATOM | 5796 | OH2 | TIP | | 206 | 82.257 | 40.382 | 21.175 | 1.00 | 36.08 |
| ATOM | 5797 | OH2 | TIP | | 207 | 75.418 | 44.375 | −1.993 | 1.00 | 40.61 |
| ATOM | 5798 | OH2 | TIP | | 208 | 8.151 | 69.166 | 16.710 | 1.00 | 44.90 |
| ATOM | 5799 | OH2 | TIP | | 209 | 83.811 | 57.022 | 19.559 | 1.00 | 23.75 |
| ATOM | 5800 | OH2 | TIP | | 210 | 89.732 | 38.502 | 10.624 | 1.00 | 45.38 |
| ATOM | 5801 | OH2 | TIP | | 211 | 57.995 | 53.900 | 13.742 | 1.00 | 45.45 |
| ATOM | 5802 | OH2 | TIP | | 212 | 41.221 | 100.360 | −7.718 | 1.00 | 49.06 |
| ATOM | 5803 | OH2 | TIP | | 213 | 72.092 | 37.629 | 32.475 | 1.00 | 61.51 |
| ATOM | 5804 | OH2 | TIP | | 214 | 37.776 | 74.115 | 23.513 | 1.00 | 53.60 |
| ATOM | 5805 | OH2 | TIP | | 215 | 95.633 | 49.033 | 19.239 | 1.00 | 39.53 |
| ATOM | 5806 | OH2 | TIP | | 216 | 15.800 | 72.932 | 22.618 | 1.00 | 47.69 |
| ATOM | 5807 | OH2 | TIP | | 217 | 63.637 | 26.806 | 29.999 | 1.00 | 42.46 |
| ATOM | 5808 | OH2 | TIP | | 218 | 48.894 | 66.556 | −18.212 | 1.00 | 40.87 |
| ATOM | 5809 | OH2 | TIP | | 219 | 76.997 | 21.646 | 4.056 | 1.00 | 37.21 |
| ATOM | 5810 | OH2 | TIP | | 220 | 86.518 | 26.866 | 20.224 | 1.00 | 45.02 |
| ATOM | 5811 | OH2 | TIP | | 221 | 44.243 | 76.229 | 10.420 | 1.00 | 38.37 |
| ATOM | 5812 | OH2 | TIP | | 222 | 25.182 | 88.366 | −6.548 | 1.00 | 37.81 |
| ATOM | 5813 | OH2 | TIP | | 223 | 99.608 | 46.589 | −2.471 | 1.00 | 52.75 |
| ATOM | 5814 | OH2 | TIP | | 224 | 64.098 | 59.015 | 25.988 | 1.00 | 34.05 |
| ATOM | 5815 | OH2 | TIP | | 225 | 49.905 | 102.416 | 19.684 | 1.00 | 44.14 |
| ATOM | 5816 | OH2 | TIP | | 226 | 91.136 | 36.314 | 11.446 | 1.00 | 47.05 |
| ATOM | 5817 | OH2 | TIP | | 227 | 81.387 | 55.146 | 27.778 | 1.00 | 47.39 |
| ATOM | 5818 | OH2 | TIP | | 228 | 60.811 | 69.016 | 15.894 | 1.00 | 48.74 |
| ATOM | 5819 | OH2 | TIP | | 229 | 60.946 | 61.261 | 26.309 | 1.00 | 54.59 |
| ATOM | 5820 | OH2 | TIP | | 230 | 33.525 | 74.075 | 4.130 | 1.00 | 51.65 |
| ATOM | 5821 | OH2 | TIP | | 231 | 85.430 | 32.491 | −10.569 | 1.00 | 31.56 |
| ATOM | 5822 | OH2 | TIP | | 232 | 12.280 | 91.312 | −6.338 | 1.00 | 37.69 |
| ATOM | 5823 | OH2 | TIP | | 233 | 29.661 | 83.836 | −4.637 | 1.00 | 33.62 |
| ATOM | 5824 | OH2 | TIP | | 234 | 68.894 | 24.672 | 2.939 | 1.00 | 31.68 |
| ATOM | 5825 | OH2 | TIP | | 235 | 84.378 | 21.827 | 27.009 | 1.00 | 38.47 |
| ATOM | 5826 | OH2 | TIP | | 236 | 61.770 | 41.536 | 29.780 | 1.00 | 28.87 |
| ATOM | 5827 | OH2 | TIP | | 237 | 22.526 | 69.316 | 12.108 | 1.00 | 56.68 |
| ATOM | 5828 | OH2 | TIP | | 238 | 72.573 | 40.681 | −4.565 | 1.00 | 41.36 |
| ATOM | 5829 | OH2 | TIP | | 239 | 64.589 | 45.566 | 4.403 | 1.00 | 21.60 |
| ATOM | 5830 | OH2 | TIP | | 240 | 39.921 | 76.650 | −25.549 | 1.00 | 42.96 |
| ATOM | 5831 | OH2 | TIP | | 241 | 37.402 | 83.028 | 26.817 | 1.00 | 45.34 |
| ATOM | 5832 | OH2 | TIP | | 242 | 33.290 | 71.184 | 24.438 | 1.00 | 41.99 |
| ATOM | 5833 | OH2 | TIP | | 243 | 20.114 | 105.283 | 26.910 | 1.00 | 42.62 |
| ATOM | 5834 | OH2 | TIP | | 244 | 72.442 | 42.860 | 29.082 | 1.00 | 42.21 |
| ATOM | 5835 | OH2 | TIP | | 245 | 13.992 | 82.149 | 1.241 | 1.00 | 38.85 |
| ATOM | 5836 | OH2 | TIP | | 246 | 41.909 | 97.922 | 17.548 | 1.00 | 41.20 |
| ATOM | 5837 | OH2 | TIP | | 247 | 72.472 | 45.709 | 8.588 | 1.00 | 21.46 |
| ATOM | 5838 | OH2 | TIP | | 248 | 70.421 | 54.773 | −0.661 | 1.00 | 43.75 |
| ATOM | 5839 | OH2 | TIP | | 249 | 67.143 | 38.463 | 0.215 | 1.00 | 36.80 |
| ATOM | 5840 | OH2 | TIP | | 250 | 87.976 | 55.453 | 18.319 | 1.00 | 28.56 |
| ATOM | 5841 | OH2 | TIP | | 251 | 69.999 | 46.346 | 31.247 | 1.00 | 39.70 |
| ATOM | 5842 | OH2 | TIP | | 252 | 54.539 | 50.747 | 4.830 | 1.00 | 37.48 |
| ATOM | 5843 | OH2 | TIP | | 253 | 21.028 | 80.517 | 26.853 | 1.00 | 44.29 |
| ATOM | 5844 | OH2 | TIP | | 254 | 18.684 | 98.982 | 25.594 | 1.00 | 37.90 |
| ATOM | 5845 | OH2 | TIP | | 255 | 70.229 | 34.803 | −1.368 | 1.00 | 60.61 |
| ATOM | 5846 | OH2 | TIP | | 256 | 87.106 | 48.869 | 19.010 | 1.00 | 32.54 |
| ATOM | 5847 | OH2 | TIP | | 257 | 47.420 | 88.249 | 7.951 | 1.00 | 35.67 |
| ATOM | 5848 | OH2 | TIP | | 258 | 21.063 | 115.438 | 17.316 | 1.00 | 35.89 |
| ATOM | 5849 | OH2 | TIP | | 259 | 24.427 | 110.948 | 2.222 | 1.00 | 34.03 |
| ATOM | 5850 | OH2 | TIP | | 260 | 52.830 | 46.305 | 6.084 | 1.00 | 39.87 |
| ATOM | 5851 | OH2 | TIP | | 261 | 42.265 | 94.805 | 19.620 | 1.00 | 24.07 |
| ATOM | 5852 | OH2 | TIP | | 262 | 56.057 | 57.733 | 27.560 | 1.00 | 42.47 |
| ATOM | 5853 | OH2 | TIP | | 263 | 19.351 | 82.738 | 26.360 | 1.00 | 35.37 |
| ATOM | 5854 | OH2 | TIP | | 264 | 23.144 | 73.194 | 30.688 | 1.00 | 47.32 |
| ATOM | 5855 | OH2 | TIP | | 265 | 63.013 | 62.906 | 25.680 | 1.00 | 38.65 |
| ATOM | 5856 | OH2 | TIP | | 266 | 41.859 | 70.029 | −12.808 | 1.00 | 43.16 |
| ATOM | 5857 | OH2 | TIP | | 267 | 41.047 | 94.499 | −12.252 | 1.00 | 30.89 |
| ATOM | 5858 | OH2 | TIP | | 268 | 49.322 | 44.383 | 24.746 | 1.00 | 57.50 |
| ATOM | 5859 | OH2 | TIP | | 269 | 14.345 | 83.254 | 27.072 | 1.00 | 47.73 |
| ATOM | 5860 | OH2 | TIP | | 270 | 84.431 | 23.892 | −19.636 | 1.00 | 50.82 |
| ATOM | 5861 | OH2 | TIP | | 271 | 7.771 | 98.423 | 13.702 | 1.00 | 46.11 |
| ATOM | 5862 | OH2 | TIP | | 272 | 13.603 | 76.350 | −2.466 | 1.00 | 47.00 |
| ATOM | 5863 | OH2 | TIP | | 273 | 43.175 | 41.193 | 4.583 | 1.00 | 40.21 |
| ATOM | 5864 | OH2 | TIP | | 274 | 58.291 | 51.532 | 7.003 | 1.00 | 54.17 |
| ATOM | 5865 | OH2 | TIP | | 275 | 51.325 | 90.886 | 14.219 | 1.00 | 42.67 |
| ATOM | 5866 | OH2 | TIP | | 276 | 86.090 | 56.149 | 23.228 | 1.00 | 40.06 |
| ATOM | 5867 | OH2 | TIP | | 277 | 50.966 | 45.969 | 21.203 | 1.00 | 32.47 |
| ATOM | 5868 | OH2 | TIP | | 278 | 89.100 | 42.135 | 3.527 | 1.00 | 47.40 |
| ATOM | 5869 | OH2 | TIP | | 279 | 17.273 | 95.041 | 1.168 | 1.00 | 43.83 |
| ATOM | 5870 | OH2 | TIP | | 280 | 58.673 | 98.407 | −7.046 | 1.00 | 45.08 |
| ATOM | 5871 | OH2 | TIP | | 281 | 78.163 | 17.076 | 4.589 | 1.00 | 46.28 |
| ATOM | 5872 | OH2 | TIP | | 282 | 101.345 | 35.952 | 7.650 | 1.00 | 41.44 |
| ATOM | 5873 | OH2 | TIP | | 283 | 58.562 | 28.216 | 31.037 | 1.00 | 37.19 |
| ATOM | 5874 | OH2 | TIP | | 284 | 3.431 | 93.236 | 20.866 | 1.00 | 46.44 |
| ATOM | 5875 | OH2 | TIP | | 285 | 46.206 | 100.458 | 11.278 | 1.00 | 42.03 |
| ATOM | 5876 | OH2 | TIP | | 286 | 7.144 | 99.468 | 5.033 | 1.00 | 60.41 |
| ATOM | 5877 | OH2 | TIP | | 287 | 56.442 | 16.403 | 16.295 | 1.00 | 48.75 |
| ATOM | 5878 | OH2 | TIP | | 288 | 72.651 | 59.077 | 36.505 | 1.00 | 45.43 |
| ATOM | 5879 | OH2 | TIP | | 289 | 15.525 | 86.890 | −5.252 | 1.00 | 45.27 |
| ATOM | 5880 | OH2 | TIP | | 290 | 52.750 | 75.991 | 4.087 | 1.00 | 52.62 |
| ATOM | 5881 | OH2 | TIP | | 291 | 48.875 | 99.238 | −12.260 | 1.00 | 44.59 |
| ATOM | 5882 | OH2 | TIP | | 292 | 46.786 | 90.537 | 21.400 | 1.00 | 39.68 |
| ATOM | 5883 | OH2 | TIP | | 293 | 85.515 | 48.612 | 7.509 | 1.00 | 34.49 |
| ATOM | 5884 | OH2 | TIP | | 294 | 7.945 | 73.643 | 11.102 | 1.00 | 42.36 |
| ATOM | 5885 | OH2 | TIP | | 295 | 18.140 | 70.001 | 15.786 | 1.00 | 43.51 |
| ATOM | 5886 | OH2 | TIP | | 296 | 54.034 | 38.692 | −2.318 | 1.00 | 46.78 |
| ATOM | 5887 | OH2 | TIP | | 297 | 58.790 | 38.984 | 30.000 | 1.00 | 42.56 |
| ATOM | 5888 | OH2 | TIP | | 298 | 53.117 | 25.322 | 21.425 | 1.00 | 48.65 |
| ATOM | 5889 | OH2 | TIP | | 299 | 79.667 | 23.687 | 28.724 | 1.00 | 31.36 |
| ATOM | 5890 | OH2 | TIP | | 300 | 65.052 | 55.747 | 26.794 | 1.00 | 43.24 |
| ATOM | 5891 | OH2 | TIP | | 301 | 82.283 | 36.493 | 26.079 | 1.00 | 36.32 |
| ATOM | 5892 | OH2 | TIP | | 302 | 27.538 | 89.554 | −2.616 | 1.00 | 41.41 |
| ATOM | 5893 | OH2 | TIP | | 303 | 55.070 | 40.595 | 29.011 | 1.00 | 38.03 |
| ATOM | 5894 | OH2 | TIP | | 304 | 101.938 | 39.946 | −7.588 | 1.00 | 47.50 |
| ATOM | 5895 | OH2 | TIP | | 305 | 94.519 | 53.023 | 16.596 | 1.00 | 46.73 |
| ATOM | 5896 | OH2 | TIP | | 306 | 4.609 | 98.144 | 17.159 | 1.00 | 47.55 |
| ATOM | 5897 | OH2 | TIP | | 307 | 72.568 | 17.727 | 20.484 | 1.00 | 43.07 |
| ATOM | 5898 | OH2 | TIP | | 308 | 44.734 | 92.751 | −18.906 | 1.00 | 41.46 |
| ATOM | 5899 | OH2 | TIP | | 309 | 39.432 | 70.028 | −8.496 | 1.00 | 43.09 |
| ATOM | 5900 | OH2 | TIP | | 310 | 78.014 | 22.700 | 12.525 | 1.00 | 38.49 |
| ATOM | 5901 | OH2 | TIP | | 311 | 15.607 | 102.239 | 7.211 | 1.00 | 40.40 |
| ATOM | 5902 | OH2 | TIP | | 312 | 62.505 | 32.788 | 30.140 | 1.00 | 27.47 |
| ATOM | 5903 | OH2 | TIP | | 313 | 16.926 | 100.528 | 24.125 | 1.00 | 42.11 |
| ATOM | 5904 | OH2 | TIP | | 314 | 88.342 | 39.723 | 21.124 | 1.00 | 27.74 |
| ATOM | 5905 | OH2 | TIP | | 315 | 66.669 | 57.207 | 0.563 | 1.00 | 37.58 |
| ATOM | 5906 | OH2 | TIP | | 316 | 59.985 | 92.952 | −1.731 | 1.00 | 41.14 |
| ATOM | 5907 | OH2 | TIP | | 317 | 75.200 | 40.815 | 31.050 | 1.00 | 46.78 |
| ATOM | 5908 | OH2 | TIP | | 318 | 89.890 | 31.775 | 17.421 | 1.00 | 43.01 |
| ATOM | 5909 | OH2 | TIP | | 319 | 36.404 | 68.064 | −5.905 | 1.00 | 56.47 |
| ATOM | 5910 | OH2 | TIP | | 320 | 84.306 | 38.435 | 22.961 | 1.00 | 30.27 |
| ATOM | 5911 | OH2 | TIP | | 321 | 37.796 | 96.408 | −12.774 | 1.00 | 61.55 |
| ATOM | 5912 | OH2 | TIP | | 322 | 19.428 | 69.397 | 2.023 | 1.00 | 33.71 |
| ATOM | 5913 | OH2 | TIP | | 323 | 57.496 | 90.636 | 1.067 | 1.00 | 54.98 |
| ATOM | 5914 | OH2 | TIP | | 324 | 12.053 | 82.679 | 19.654 | 1.00 | 38.70 |
| ATOM | 5915 | OH2 | TIP | | 325 | 88.445 | 13.028 | 2.038 | 1.00 | 43.48 |
| ATOM | 5916 | OH2 | TIP | | 326 | 69.293 | 34.255 | −4.128 | 1.00 | 36.68 |
| ATOM | 5917 | OH2 | TIP | | 327 | 70.958 | 38.574 | −3.994 | 1.00 | 40.96 |
| ATOM | 5918 | OH2 | TIP | | 328 | 85.340 | 60.784 | 14.081 | 1.00 | 43.08 |
| ATOM | 5919 | OH2 | TIP | | 329 | 15.966 | 91.329 | −4.428 | 1.00 | 61.14 |
| ATOM | 5920 | OH2 | TIP | | 330 | 20.245 | 87.313 | 26.799 | 1.00 | 46.63 |
| ATOM | 5921 | OH2 | TIP | | 331 | 4.489 | 97.007 | 19.737 | 1.00 | 56.45 |
| ATOM | 5922 | OH2 | TIP | | 332 | 85.224 | 40.207 | 1.203 | 1.00 | 40.83 |
| ATOM | 5923 | OH2 | TIP | | 333 | 33.134 | 94.962 | −3.857 | 1.00 | 32.00 |
| ATOM | 5924 | OH2 | TIP | | 334 | 81.535 | 66.877 | 20.224 | 1.00 | 41.87 |
| ATOM | 5925 | OH2 | TIP | | 335 | 22.505 | 114.686 | 12.039 | 1.00 | 42.78 |
| ATOM | 5926 | OH2 | TIP | | 336 | 51.440 | 46.909 | 13.064 | 1.00 | 66.48 |
| TER | 5927 | | TIP | | 336 | | | | | |
| ATOM | 5928 | C1 | GOL | | 529 | 79.436 | 25.570 | 17.948 | 1.00 | 44.78 |
| ATOM | 5929 | O1 | GOL | | 529 | 80.194 | 24.757 | 17.061 | 1.00 | 44.32 |
| ATOM | 5930 | C2 | GOL | | 529 | 77.910 | 25.395 | 17.624 | 1.00 | 46.09 |
| ATOM | 5931 | O2 | GOL | | 529 | 77.242 | 24.886 | 18.794 | 1.00 | 45.98 |
| ATOM | 5932 | C3 | GOL | | 529 | 77.690 | 24.402 | 16.446 | 1.00 | 47.19 |
| ATOM | 5933 | O3 | GOL | | 529 | 76.335 | 24.396 | 15.965 | 1.00 | 45.74 |
| ATOM | 5934 | C1 | GOL | | 530 | 78.715 | 26.772 | 9.652 | 1.00 | 54.03 |
| ATOM | 5935 | O1 | GOL | | 530 | 79.769 | 27.605 | 10.132 | 1.00 | 51.36 |
| ATOM | 5936 | C2 | GOL | | 530 | 79.284 | 25.356 | 9.258 | 1.00 | 55.43 |
| ATOM | 5937 | O2 | GOL | | 530 | 78.624 | 24.336 | 10.032 | 1.00 | 55.99 |
| ATOM | 5938 | C3 | GOL | | 530 | 80.810 | 25.270 | 9.508 | 1.00 | 56.69 |
| ATOM | 5939 | O3 | GOL | | 530 | 81.303 | 23.931 | 9.405 | 1.00 | 58.79 |
| ATOM | 5940 | C1 | GOL | | 531 | 79.935 | 36.623 | 23.917 | 1.00 | 28.32 |
| ATOM | 5941 | O1 | GOL | | 531 | 79.999 | 38.046 | 24.006 | 1.00 | 31.93 |
| ATOM | 5942 | C2 | GOL | | 531 | 78.449 | 36.144 | 24.139 | 1.00 | 29.57 |
| ATOM | 5943 | O2 | GOL | | 531 | 77.997 | 35.405 | 22.997 | 1.00 | 29.88 |
| ATOM | 5944 | C3 | GOL | | 531 | 77.481 | 37.343 | 24.380 | 1.00 | 29.38 |
| ATOM | 5945 | O3 | GOL | | 531 | 76.119 | 36.913 | 24.548 | 1.00 | 26.36 |
| ATOM | 5946 | C1 | GOL | | 532 | 79.310 | 52.604 | 22.101 | 1.00 | 21.78 |
| ATOM | 5947 | O1 | GOL | | 532 | 79.210 | 53.188 | 20.791 | 1.00 | 17.99 |
| ATOM | 5948 | C2 | GOL | | 532 | 78.806 | 51.109 | 22.092 | 1.00 | 14.64 |
| ATOM | 5949 | O2 | GOL | | 532 | 79.917 | 50.290 | 22.447 | 1.00 | 18.34 |
| ATOM | 5950 | C3 | GOL | | 532 | 78.316 | 50.657 | 20.683 | 1.00 | 21.57 |
| ATOM | 5951 | O3 | GOL | | 532 | 77.640 | 49.388 | 20.678 | 1.00 | 14.27 |
| ATOM | 5952 | C1 | GOL | | 533 | 12.964 | 90.743 | 6.167 | 1.00 | 35.75 |
| ATOM | 5953 | O1 | GOL | | 533 | 14.136 | 90.064 | 6.618 | 1.00 | 40.00 |
| ATOM | 5954 | C2 | GOL | | 533 | 13.372 | 92.168 | 5.655 | 1.00 | 35.48 |
| ATOM | 5955 | O2 | GOL | | 533 | 13.004 | 92.274 | 4.266 | 1.00 | 32.20 |
| ATOM | 5956 | C3 | GOL | | 533 | 14.904 | 92.393 | 5.800 | 1.00 | 31.71 |
| ATOM | 5957 | O3 | GOL | | 533 | 15.276 | 93.767 | 5.694 | 1.00 | 28.86 |
| ATOM | 5958 | C1 | GOL | | 534 | 55.733 | 40.012 | 9.098 | 1.00 | 22.83 |
| ATOM | 5959 | O1 | GOL | | 534 | 56.861 | 39.435 | 9.738 | 1.00 | 24.61 |
| ATOM | 5960 | C2 | GOL | | 534 | 56.230 | 41.193 | 8.164 | 1.00 | 25.10 |
| ATOM | 5961 | O2 | GOL | | 534 | 55.900 | 40.826 | 6.823 | 1.00 | 20.99 |
| ATOM | 5962 | C3 | GOL | | 534 | 57.766 | 41.387 | 8.242 | 1.00 | 23.90 |
| ATOM | 5963 | O3 | GOL | | 534 | 58.168 | 42.742 | 8.018 | 1.00 | 25.49 |
| TER | 5964 | | GOL | | 534 | | | | | |

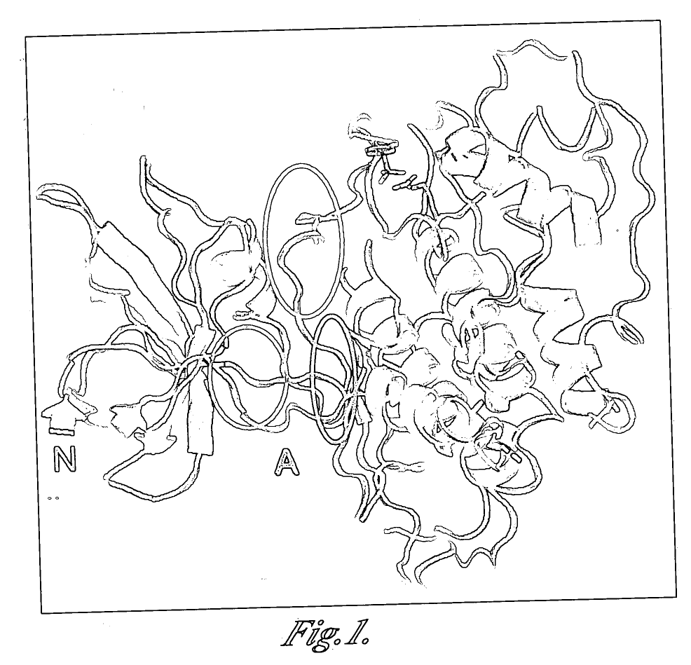

The three-dimensional structure of the GSK3-β construct SEQ ID NO:1 based on the derived crystal structure is schematically illustrated in FIG. 1. The construct includes N-terminal and C-terminal domains with the active site formed between the two domains. The N-terminal domain includes a β-barrel. The active site region includes the ATP binding site, the magnesium binding/catalytic base site, and substrate binding site.

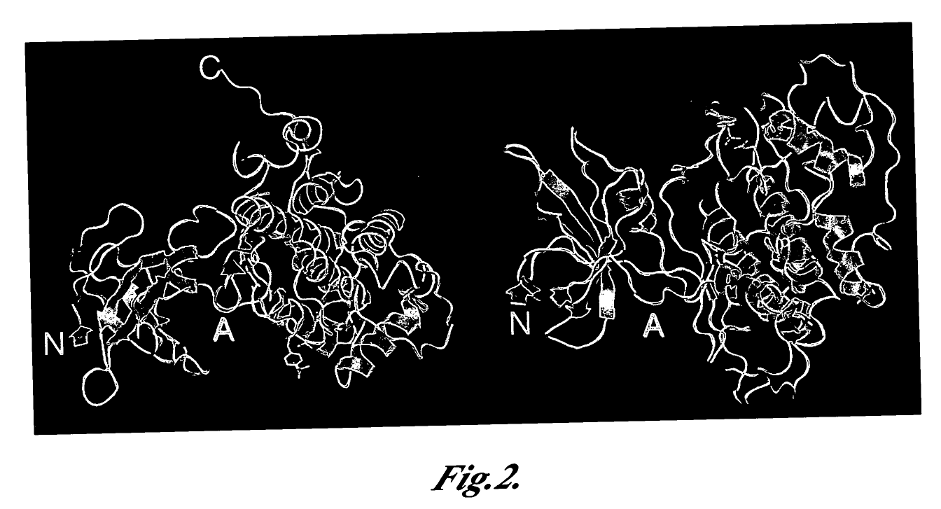

The three-dimensional structure of the GSK3-β construct's SEQ ID NO:1 active site (including the catalytic site and substrate binding site) based on the derived crystal structure is schematically illustrated in FIG. 2. The active site includes Pro 136 and Phe67 among other amino acid residues.

Structural information of the apoprotein active site can provide a basis for the rational design of ligands leading to therapeutic compounds effective in the treatment of various disease conditions mediated by GSK3-β activity. Thus, the structural information obtained from the crystallographic data can be used to develop a ligand profile and for the rational design of drugs for mediating GSK3-β activity as described below.

GSK3 Structural Representation. As noted above, in one aspect, the invention provides a method for identifying possible therapeutic compounds in the treatment of various disease conditions mediated by GSK3-β activity. The method involves the use of a three-dimensional structural representation of the GSK construct SEQ ID NO:1. The three-dimensional structural representation may be a representation of (a) the complete GSK construct SEQ ID NO:1, (b) a fragment of GSK3 that includes the GSK construct SEQ ID NO:1, or (c) a fragment of the GSK construct SEQ ID NO:1 that includes the amino acids that interact with ligands that can mediate GSK3 activity.

The structural representation is preferably based on or derived from the atomic coordinates as set out in Table 2, which represents the structure of the complete GSK construct. Suitable structural representations include three-dimensional models and molecular surfaces derived from these atomic coordinates. The coordinates in Table 2 include structural water and glycerol molecules. These will vary, and may even be absent, in other models derived structurally (they are resolution and space group dependent). These solvent molecules will vary from crystal to crystal.

Variants of the atomic coordinates noted in Table 2 can also be used for the invention, such as variants in which the RMS deviation of the x, y, and z coordinates for all heavy (i.e., not hydrogen) atoms are less than about 2.5 Å, for example, less than about 2 Å, preferably less than about 1 Å, more preferably less than about 0.5 Å, or most preferably less than about 0.1 Å) compared with the atomic coordinates noted in Table 2. Coordinate transformations that retain the three-dimensional spatial relationships of atoms can also be used to give suitable variants.

The atomic coordinates provided herein can also be used as the basis of models of further protein structures. For example, a homology model could be based on the GSK construct SEQ ID NO:1 structure. The coordinates can also be used in the solution or refinement of further crystal structures of GSK3, such as co-crystal structures with new ligands.

GSK3 Structural Representation Storage Medium. The atomic coordinates of the GSK construct SEQ ID NO:1 can be stored on a medium for subsequent use with a computational device, such as a computer (e.g., supercomputer, mainframe, minicomputer, or microprocessor). Typically, the coordinates are stored on a medium useful to hold large amounts of data, such as magnetic or optical media (e.g., floppy disks, hard disks, compact disks, magneto-optical media (“floptical” disks, or magnetic tape) or electronic media (e.g., random-access memory (RAM), or read-only memory (ROM). The storage medium can be local to the computer, or can be remote (e.g., a networked storage medium, including the Internet). The choice of computer, storage medium, networking, and other devices or techniques will be familiar to those of skill in the structural/computational chemistry arts.

The invention also provides a computer-readable medium for a computer, which contains atomic coordinates and/or a three-dimensional structural representation of the GSK construct SEQ ID NO:1. The atomic coordinates are preferably those noted in Table 2 or variants thereof. Any suitable computer can be used in the present invention.

GSK3-β Ligand Profile Development. As noted above, the structural information obtained from the crystallographic data can be used to develop a ligand profile useful for the rational design of compounds for mediating GSK3-β activity. A ligand profile can be developed by taking into account the structural information obtained as described above for the apoprotein. The ligand profile can be further developed and refined with the determination of additional structures of protein with bound ligands. The ultimately developed ligand profile identifies possible therapeutic compounds for mediating GSK3-β activity.

The ligand profile can be primarily based on a shape interaction between the ligand and the protein ligand binding site. The evaluation of the shape interaction can include consideration of the ligand's conformational properties, ranking ligands based on their ability to achieve low energy conformations compatible with the ligand binding site. The shape interaction can also seek to maximize enthalpic interactions between the ligand and the binding site.

The process of developing a ligand profile can vary widely. For example, the profile can be developed by visual inspection of active site structures by experts. Such an inspection can include the consideration of the binding site and ligand structures and compound database searching. The development of the profile can also consider biological data and structure activity relationships (SAR) as well the consideration of known ligand binding interaction with other similar proteins.

In any event, the ligand profile is developed by considering ligand binding interactions including primary and secondary interactions and results in defining the pharmacophore. The term “pharmacophore” refers to a collection of chemical features three-dimensional constraints that represent specific characteristics responsible for a ligand's activity. The pharmacophore includes surface-accessible features, hydrogen bond donors and acceptors, charged/ionizable groups, and/or hydrophobic patches, among other features.

In addition to the process for ligand profile development noted above, other structure-based drug design techniques can be applied to the structural representation of the GSK3 construct SEQ ID NO:1 in order to identify compounds that interact with GSK3 to mediate GSK3 activity. A variety of suitable techniques are available to one of ordinary skill in the art.

Software packages for implementing molecular modeling techniques for use in structure-based drug design include SYBYL (available from Tripos Inc.); AMBER (available from Oxford Molecular); CERIUS² (available from Molecular Simulations Inc.); INSIGHT II (available from Molecular Simulations Inc.) CATALYST (available from Molecular Simulations Inc.); QUANTA (available from Molecular Simulations Inc.); HYPERCHEM (available from Hypercube Inc.); FIRST DISCOVERY (available from Schrodinger Inc.), MOE (available from Chemical Computing Group), and CHEMSITE (available from Pyramid Learning), among others.

The modeling software can be used to determine GSK3 binding surfaces and to reveal features such as van der Waals contacts, electrostatic interactions, and/or hydrogen bonding opportunities. These binding surfaces can be used to model docking of ligands with GSK3, to arrive at pharmacophore hypotheses, and to design possible therapeutic compounds de novo.

GSK3-β Ligand Virtual Screening

The three-dimensional structure of the apoprotein, and the structure of the protein's active site in particular, allows for the determination of the fit of compounds into the active site. Utilizing a fast docking program, individual compounds from, for example, a compound database, can be evaluated for active site binding. The fit of a particular compound can be evaluated and scored. Setting a score threshold can then provides a family of compounds as a solution to the virtual screen.

At the first level, the virtual screen takes into account the three-dimensional structure of the apoprotein's active site. At the second level, the virtual screen considers the ligand profile and can utilize information obtained from the determination of the structure of protein with bound ligand. A virtual screen is possible even if there is no structural information on a bound ligand.

Information gained from the virtual screen can be considered to further develop the ligand profile. Alternatively, where the results of the virtual screen indicate a promising compound, the compound can be obtained and screened for the relevant biological activity.

Docking. Docking refers to a process in which two or more molecules are aligned based on energy considerations. Docking aligns the three-dimensional structures of two or more molecules to predict the conformation of a complex formed from the molecules (see, e.g., Blaney & Dixon (1993) Perspectives in Drug Discovery and Design 1:301). In the practice of the present invention, molecules are docked with the GSK3 construct SEQ ID NO:1 structure to assess their ability to interact with GSK3.

Docking can be accomplished by either geometric matching of the ligand and its receptor or by minimizing the energy of interaction. Geometric matching algorithms are preferred because of their relative speed.

Suitable docking algorithms include DOCK (Kuntz et al. (1982) J. Mol. Biol. 161:269-288, available from UCSF), the prototypical program for structure-based drug design; AUTODOCK (Goodsell & Olson (1990) Proteins: Structure, Function and Genetics 8:195-202 and available from Oxford Molecular), which docks ligands in a flexible manner to receptors using grid-based Monte Carlo simulated annealing. The flexible nature of the AUTODOCK procedure helps to avoid bias (e.g., in orientation and conformation of the ligand in the active site) introduced by the user searcher (Meyer et al. (1995) Persp. Drug Disc. 3:168-95) because, while the starting conformation in a rigid docking is normally biased towards a minimum energy conformation of the ligand, the binding conformation may be of relatively high conformational energy (Nicklaus et al. (1995) Bioorganic & Medicinal Chemistry 3:411).

Other suitable docking algorithms include MOE-DOCK (available from Chemical Computing Group Inc.), in which a simulated annealing search algorithm is used to flexibly dock ligands and a grid-based energy evaluation is used to score docked conformations; FLExX (available from Tripos Inc.), which docks conformationally flexible ligands into a binding site using an incremental construction algorithm that builds the ligand in the site, and scores docked conformations based on the strength of ligand-receptor interactions; GOLD (Jones et al. (1997) J. Mol. Biol. 267:727-748), a genetic algorithm for flexible ligand docking, with full ligand and partial protein flexibility, and in which energy functions are partly based on conformation and non-bonded contact information; AFFINITY (available from Molecular Simulations Inc.), which uses a two step process to dock ligands: first, initial placements of the ligand within the receptor are made using a Monte Carlo-type procedure to search both conformational and Cartesian space; and second, a simulated annealing phase optimizes the location of each ligand placement, during this phase, AFFINITY holds the “bulk” of the receptor (atoms not in the binding site) rigid, while the binding site atoms and ligand atoms are movable; C² LigandFit (available from Molecular Simulations Inc.), which uses the energy of the ligand-receptor complex to automatically find best binding modes and stochastic conformation search techniques, with the best results from the conformational sampling retained. A grid method is used to evaluate non-bonded interactions between the rigid receptor and the flexible ligand atoms. DOCKIT (available from Metaphorics LLC) uses distance geometry for fast flexible ligand docking. GLIDE (available from Schrodinger Inc.) uses a pre-computed energy grid and an efficiently pruned systematic search for flexible docking.

Preferably, the docking algorithm is used in a high-throughput mode, in which members of large structural libraries of potential ligands are screened against the receptor structure (Martin (1992) J. Med. Chem. 35:2145-54).

Suitable structural libraries include the ACD (Available Chemical Directory, form MDL Inc.), AsInEx, Bionet, ComGenex, the Derwent World Drug Index (WDI), the Contact Service Company database, LaboTest, ChemBridge Express Pick, ChemStar, BioByteMasterFile, Orion, SALOR, TRIAD, ILIAD, the National Cancer Institute database (NCI), and the Aldrich, Fluka, Sigman and Mabridge catalogs. These are commercially available (e.g., the HTS Chemicals collection from Oxford Molecular, or the LeadQuest™ files from Tripos).

Defining the Pharmacophore. As noted above, a pharmacophore can be defined for the GSK3 construct SEQ ID NO:1 that includes surface-accessible features, hydrogen bond donors and acceptors, charged/ionizable groups, and/or hydrophobic patches, among other features. These features can be weighted depending on their relative importance in conferring activity (see, e.g., Computer-Assisted Lead Finding and Optimization (eds. Testra & Folkers, 1997).

Pharmacophores can be determined using software such as CATALYST (including HypoGen or HipHop, available from Molecular Simulations Inc.), CERIUS², or constructed by hand from a known conformation of a lead compound. The pharmacophore can be used to screen structural libraries, using a program such as CATALYST. The CLIX program (Davic & Lawrence (1992) Proteins 12:31-41) can also be used, which searches for orientations of candidate molecules in structural databases that yield maximum spatial coincidence with chemical groups which interact with the receptor. The DISCO program (available from Tripos) uses a method of clique detection to identify common pharmacophoric features in each structure, produce optimally aligned structures, and extract the key features of the pharmacophore. The GASP program (available from Tripos) uses a genetic algorithm to automatically find pharmacophores with conformational flexibility.

de novo Compound Design. The binding surface or pharmacophore of the GSK3 construct SEQ ID NO:1 can be used to map favorable interaction positions for functional groups (e.g., protons, hydroxyl groups, amine groups, acidic groups, hydrophobic groups and/or divalent cations) or small molecule fragments. Compounds can then be designed de novo in which the relevant functional groups are located in the correct spatial relationship to interact with GSK3.

Once functional groups or small molecule fragments which can interact with specific sites in the binding surface of GSK3 have been identified, they can be linked in a single compound using either bridging fragments with the correct size and geometry or frameworks which can support the functional groups at favorable orientations, thereby providing a compound according to the invention. While linking of functional groups in this way can be done manually, perhaps with the help of software such as QUANTA or SYBYL, automated or semi-automated de novo design approaches can also be used.

Suitable de novo design software includes MCDLNG (Gehlhaar et al. (1995) J. Med. Chem. 38:466-72), which fills a receptor binding site with a close-packed array of generic atoms and uses a Monte Carlo procedure to randomly vary atom types, positions, bonding arrangements and other properties; MCSS/HOOK (Caflish et al. (1993) J. Med. Chem. 36:2142-67; Eisen et al. (1994) Proteins: Str. Funct. Genet. 19:199-221; available from Molecular Simulations Inc.), which links multiple functional groups with molecular templates taken from a database; LUDI (Bohm (1992) J. Comp. Aided Molec. Design 6:61-78, available from Molecular Simulations Inc.), which computes the points of interaction that would ideally be fulfilled by a ligand, places fragments in the binding site based on their ability to interact with the receptor, and then connects them to produce a ligand; GROW (Moon and Howe (1991) Proteins: Str. Funct. Genet. 11:314-328), which starts with an initial “seed” fragment (placed manually or automatically) and grows the ligand outwards; SPROUT which includes molecules to identify favorable hydrogen bonding and hydrophobic regions within a binding pocket (HIPPO module), selects functional groups and positions them at target sites to form starting fragments for structure generation (EleFanT), generates skeletons that satisfy the steric constraints of the binding pocket by growing spacer fragments onto the start fragments and then connecting the resulting part skeletons (SPIDeR), substitutes hetero atoms into the skeletons to generate molecules with the electrostatic properties that are complementary to those of the receptor site (MARABOU), and the solutions can be clustered and scored using the ALLigaTOR module; LEAPFROG (available from Tripos Inc.), which evaluates ligands by making small stepwise structural changes and rapidly evaluating the binding energy of the new compound, keeps or discards changes based on the altered binding energy, and evolves structures to increase the interaction energy with the receptor; GROUPBUILD (Rorstein et al. (1993) J. Med. Chem. 36:1700), which uses a library of common organic templates and a complete empirical force field description of the non-bonding interactions between a ligand and receptor to construct ligands that have chemically reasonable structure and have steric and electrostatic properties complimentary to the receptor binding site; CAVEAT (Lauri and Bartlett (1994) Comp. Aided Mol. Design. 8:51-66), which designs linking units to constrain acyclic molecules; and RASSE (Lai (1996) J. Chem. Inf. Comput. Sci. 36:1187-1194).

GSK3-β Ligands

Most lead compounds that initiate structure-based design cycles are identified by high-throughput screening. As a result of high throughput screening and the ligand profile and virtual screening described above, ligands are identified having the requisite conformational energies to assume a suitable shape and bind with the protein's active site. In addition to having low conformational energy and spatial compatibility with the apoprotein active site, the identified ligands are preferably synthetically accessible. The identified ligands can then be obtained (e.g., commercially obtained or synthesized) and screened for biological activity. The identified ligands can also be co-crystallized with the protein construct and the three-dimensional structure determined for the protein with bound ligand. The information obtained from structure of the protein with bound ligand can then be used to further develop the ligand profile as described above.

Suitable GSK3-β biological screening methods for evaluating ligand biological activity are known and include, for example, those noted in U.S. patent application Ser. No. 60/193,043, filed Mar. 29, 2000, and expressly incorporated herein be reference in its entirety.

Method for Rational Drug Discovery Using GSK3 Crystal Structures

In another aspect, the invention provides a method for using a GSK3 crystal structure, specifically the three-dimensional structure of the GSK3 construct's SEQ ID NO:1 active site, to design ligands for binding to and mediating the activity of GSK3-β.

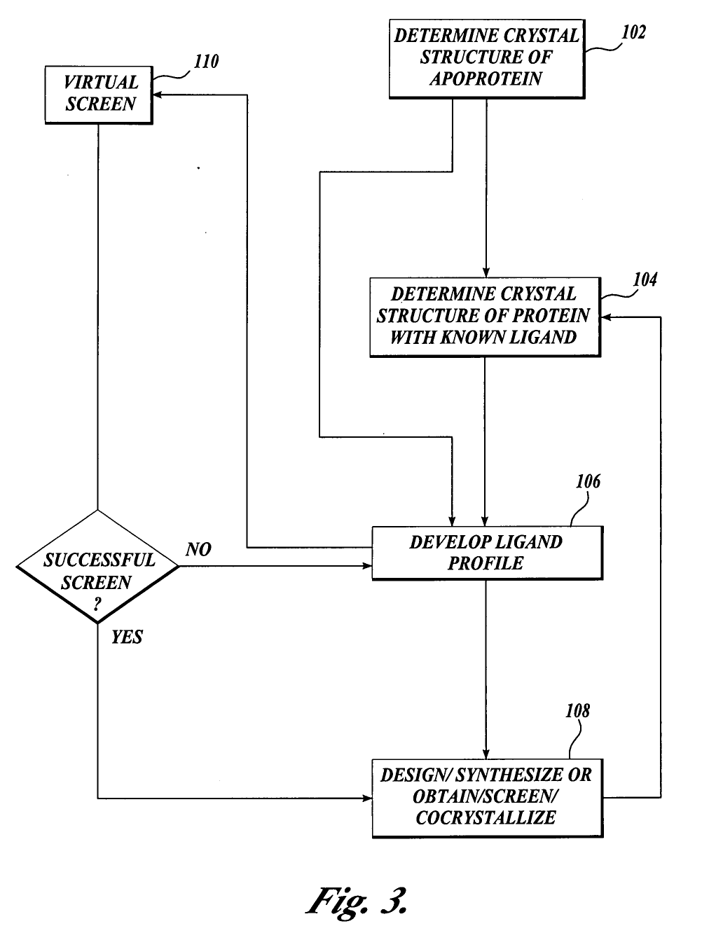

In one embodiment, the method is an iterative structure-based method for therapeutic compound design. A representative method is depicted by the flow diagram shown in FIG. 3. Referring to FIG. 3, the crystal structures of the apoprotein and the protein with bound ligand are determined in steps 102 and 104, respectively. From the structural information obtained from steps 102 and 104, a ligand profile is developed in step 106. A ligand profile can also be developed directly from the crystal structure of the apoprotein. Using the resulting profile, new ligands can be designed and/or obtained, screened for biological activity, and/or co-crystallized with the protein in step 108, or alternatively, the ligand profile can be used in a virtual screen in step 110. If the ligand obtained from the developed profile is co-crystallized, the structure of the co-crystal is determined in step 104 and the resulting structural information is used to further develop the ligand profile in step 106. If the ligand profile is used in a virtual screen in step 110, the virtual screen is either successful and identifies one or more ligands that can be obtained, screened, and/or co-crystallized in step 108. If the virtual screen is unsuccessful in identifying a suitable ligand, the ligand profile is further developed in step 106.

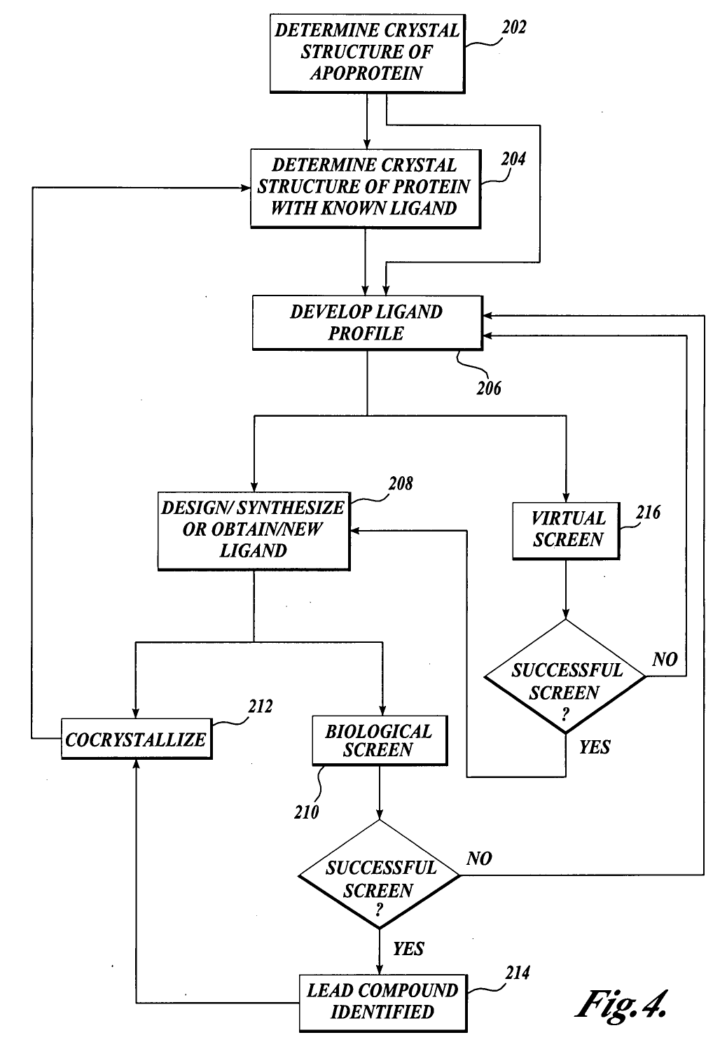

Lead compounds can be identified from biological screening of ligands developed by the ligand profile. A representative method for identifying a lead compound is depicted by the flow diagram shown FIG. 4. Referring to FIG. 4, the crystal structures of the apoprotein and the protein with bound ligand are determined in steps 202 and 204, respectively. From the structural information obtained from steps 202 and 204, a ligand profile is developed in step 206. A ligand profile can also be developed directly from the crystal structure of the apoprotein. From the resulting profile, a new ligand can be designed and/or obtained in step 208, and either screened for biological activity in step 210 and/or co-crystallized with the protein in step 212. If the biological screen is successful, a lead compound is identified in step 214. In a subsequent iteration, the lead compound can be co-crystallized in step 212 and iterations continued until a new drug candidate is identified. If the biological screen is not successful, that information can be used to further develop the ligand profile in step 206. If the ligand is co-crystallized, the co-crystal structure can be determined in step 204 and the structural information used in further developing the ligand profile in step 206.

Alternatively, the results of the ligand profile can be used in a virtual screen in step 216. If the virtual screen is successful and identifies one or more ligands, the ligand can be obtained in step 208 and screened in step 210 to determine its biological activity and whether or not a lead compound has been identified. The ligand obtained in step 208 can also be co-crystallized in step 212 and its structure determined and the resulting information used to further develop the ligand profile. If the virtual screen is unsuccessful in identifying a suitable ligand, the ligand profile is further developed in step 206.

GSK3 Ligands and their Uses

The method of the invention identifies ligands that can interact with GSK3. These compounds can be designed de novo, can be known compounds, or can be based on known compounds. The compounds can be useful pharmaceuticals themselves, or can be prototypes that can be used for further pharmaceutical refinement (i.e., lead compounds) in order to improve binding affinity or other pharmacologically important features (e.g., bio-availability, toxicology, metabolism, pharmacokinetics).

Accordingly, in another aspect, the invention provides (1) a compound identified using the method of the invention; (2) a compound identified using the method of the invention for use as a pharmaceutical; (3) the use of a compound identified using the method of the invention in the manufacture of a medicament for mediating GSK3 activity; and (4) a method of treating a patient afflicted with a condition mediated by GSK3 activity that includes administering an amount of a compound identified using the method of the invention that is effective to mediate GSK3 activity.

These compounds preferably interact with GSK3 with a binding constraint in the micromolar or, more preferably, nanomolar range or stronger.

As well as being useful compounds individually, ligands identified by the structure-based design techniques can also be used to suggest libraries of compounds for traditional in vitro or in vivo screening methods. Important pharmaceutical motifs in the ligands can be identified and mimicked in compound libraries (e.g., combinatorial libraries) for screening for GSK3-binding activity.

The foregoing and other aspects of the invention may be better understood in connection with the following representative examples.

### EXAMPLES

### Example 1

### GSK3-β Construct SEQ ID NO:1 Purification

In this example, the purification of the GSK3-β protein construct SEQ ID NO:1 is described. The construct was extracted from SF-9 cells infected with a baculovirus carrying GSK3-β 580 cDNA construct SEQ ID NO:1 and purified to apparent homogeneity using S-Fractogel, Phenyl-650 M, and Glu-tag affinity chromatographies as described below.

Extraction. Cell paste from 20 L fermentation of infected SF-9 cells was washed 100 mL PBS (10 mM NaPi, pH 7.5, 150 mM NaCl) and then resuspended with 300 mL of Buffer H (20 mM Tris, pH 7.5, 1 mM tungstate, 1 mM arsenate, 50 mM DTT, 10 μg/mL leupeptin, 1 μg/mL pepstatin A, 10% glycerol, 0.35% octyl glycoside, 1 mM Mg²⁺). Cells were homogenized in a 100-mL Douncer (20 strokes with pestel B). The combined homogenate was centrifuged in a Ti45 rotor at 40,000 rpm for 35 minutes to remove cell debris and nuclei. The supernatant from the centrifugation were carefully decanted and filtered through 0.45μ filter.

S-Fractogel Chromatography. 175 mL S-fractogel (EM Science, Cat #18882) was packed into 5 cm×8.9 cm column and equilibrated with 5 column volumes of Buffer A (20 mM Tris, Ph 7.5, 10% glycerol). Prior to loading the filtered supernatant, one column volume of Buffer A containing 50 mM DTT was passed over the equilibrated column. The filtrate from the previous step was then loaded at 20 mL/min onto the column. The column was washed with 3 column volumes of Buffer A containing 50 mM DTT and 2 column volumes of Buffer A and then eluted with a linear gradient from 0 to 1 M NaCl in Buffer A over 20 column volumes. The eluant was fractionated into 20 mL fractions. Fractions containing GSK3 were detected by Western Blot using anti-GSK antibody (Santa Cruz Biotech, Cat # SC-7291). The Western-Blot positive fractions were pooled and mixed with equal volume of Buffer M (20 mM Tris, pH 7.5, 10% glycerol, 3.1 M NaCl) and filtered through a 0.45μ filter. The filtrate was used for Phenyl-650 M chromatography.

Phenyl-650 M Chromatography. 37.5 mL Phenyl-650 M (Tosohass, Cat #014943) was packed into a 2.2×10 cm column and equilibrated with 5 column volumes of Buffer C (20 mM Tris, pH 7.5, 10% glycerol, 1.6 M NaCl). Filtrate from S-fractogel step was loaded onto the column at 7.5 mL/min. After the loading was completed, the column was washed with 5 column volumes of Buffer C and eluted with linear gradient from 0% to 100% Buffer A (20 mM Tris, pH 7.5, 10% glycerol) over 20 column volumes. Fractions were collected at 15 mL each and GSK containing fractions were detected by Western Blot using anti-GSK antibody. The Western positive fractions were pooled and loaded onto a Glu-tag antibody affinity column.

Glu-tag Affinity Chromatography. 50 mg of Glu-tag antibody was immobilized onto 28 mL of Affi-Gel 10 (BioRAD, Cat #153-6046) and the packed into 2.2×6.5 cm column. The column was equilibrated with 5 column volumes of Buffer D (20 mM Tris, pH 7.5, 20% glycerol, 0.3 M NaCl, 0.2% octylglucoside) and the fraction pool from Phenyl-650 M step was loaded at 2.8 mL/min. After the loaded was completed, the column was wash with 5 column volumes of Buffer D and then eluted with 100 mL Glu-tag peptide (100 μg/mL) in Buffer D and fractionated into 4 mL fractions. GSK containing fractions were detected with SDS-PAGE and Coomassie Blue staining. These fractions were pooled, concentrated, and diafiltered into Buffer D to approximately 4.8 mg/mL in an Amicon concentrator using a 10 k MWCO YM10 membrane. The concentrated material was then submitted for crystallization.

### Example 2

### GSK3-β Construct SEQ ID NO:1 Crystallization

In this example, the crystallization of the GSK3-β protein construct SEQ ID NO:1 is described. Crystallization protocol utilizes GSK3β at a concentration of 4.8 mg/ml and in a solution containing 1× tris buffered saline (TBS), 0.3 M NaCl, 20% glycerol, 0.2% octyl glucopyranoside, 5 mM DTT. The crystals were grown by the vapor diffusion using the hanging drop method. Briefly, each well of a 24-well linbro culture plate was filled with 0.5 mL of a solution containing 10% polyethylene glycol 6000 (PEG 6000), 5% 2-methyl-2,4-pentanediol (MPD), 0.1 M HEPES, pH 7.5. 3 uL of protein solution was then mixed with 3 uL of well solution in a drop on a glass cover slip. The cover slip was then placed over the reservoir of the well. The plate was then stored at 4° C. Crystals appeared in 50-90% of the wells in 3-30 days.

Representative GSK3 crystals are highly polymorphic and exhibit variance in both the space-group and unit cell dimensions, often showing space group transitions during compounds soaking and in response to subtle changes in conditions. Three space groups have been identified in GSK3 crystals and are given below. The space group dimensions in all the space groups and the beta angle in space group C2 can often vary by as much as 10% between individual crystals. The space group and dimensions are as follows. C222(1) is the most prevalent space group, followed by C2, and then P222(1). C2 is seen only when a compound has been soaked into the crystal.

C222(1): a=89.874 b=102.514 c=108.194 alpha=90 beta-90 gamma=90

C2: a=102.366 b=89.598 c=110.438 alpha=90 beta=96.983 gamma=90

P222(1): a=86.328 b=103.061 c=102.846 alpha=90 beta=90 gamma=90

The crystals can be cryoprotected for data collection in a cryosolution consisting of 12% PEG 6000, 11% MPD, 0.1 M HEPES pH 7.5, 20% glycerol. The cryosolution can include from about 10 to about 14 percent by weight polyethylene glycol (PEG 6000), from about 9 to about 13 percent by weight 2-methyl-2,4-pentanediol (MPD), and from about 18 to about 22 percent by weight glycerol. The cryosolution can have a pH of from about 7.3 to about 7.7.

### Example 3

### GSK3-β Construct SEQ ID NO:1 Crystal Structure Resolution

In this example, a representative method for resolving the crystal structure of the GSK3-β protein construct SEQ ID NO:1 is described.

The crystal structure of GSK3β was solved using a combination of molecular replacement and cross-crystal averaging. A molecular replacement solution for the C222(1) crystal form was obtained using a homology model of GSK3β (containing residues 53-352) and the program EPMR [C R Kissinger, D K Gehlhaar, and D B Fogel. Rapid automated molecular replacement by evolutionary search Acta Crystallogr D Biol Crystallogr. 1999 February; 55 (Pt 2):484-91]. The homology model was constructed using the phosphorylated form of CDK2 (PDB accession code 1JST) as the template. This initial molecular replacement solution gave a model with an R-factor of 53.9%. The model was then processed through several rounds of a refinement macrocycle. A typical refinement macrocycle consists of 100-200 rounds of conjugate gradient minimization, simulated annealing with either torsion or Cartesian dynamics, and grouped or individual temperature factor calculation. All refinement procedures were executed using the program CNX (Molecular Simulation, Inc.) This was followed by calculation of new electron density maps and manual rebuilding of the model based on features within these maps using the program O (DATAONO AB). All data from 30 Å-2.8 Å in the C222(1) set of data was used. Despite numerous macrocycles of refinement, it was not possible to improve the model to better than an R-factor of 35% (free-R factor of 0.41). This bias towards an incorrect structure was caused by the lack of high-resolution data and the low ratio of parameters to measurable data in the refinement.

To alleviate this bias, cross-crystal averaging was used to improve the phases provided by the initial molecular replacement solution. In cross-crystal averaging, two or more separate “views” of a structure are obtained from crystals of vastly different unit cell dimensions and/or space-groups. The electron densities of these views are then averaged together to provide more accurate phases for the Fourier reconstruction of the electron density and an improved view of the actual electron density within each of the separate crystals. For the cross-crystal averaging, the C222(1) crystal data set and C2 crystal data set (obtained from soaking a GSK3β crystal in 1-3 mM of Compound 1 (illustrated below) were used. Two molecular replacement solutions were obtained for the C2 crystal form using the partially refined GSK3β model; these solutions represented the two molecules of GSK3β in the asymmetric unit of the C2 cell. The real-space transforms between the molecule in the C222(1) crystal form and the molecules in the C2 crystal form were then calculated. Following this, the phases and figures-of-merit from an ensemble of 10 structures run through the refinement macrocycle in the C222(1) crystal form were averaged against the data from the C2 crystal form using the program DMMULTI [K. Cowtan. Joint CCP4 and ESF-EACBM Newsletter on Protein Crystallography 1994; 31, pp 34-38] and all data to 2.7 Å. One hundred cycles of averaging, solvent-flattening, and histogram matching were performed. Following this, improved electron density maps were calculated and the model was rebuilt, followed by another refinement macrocycle, and another round of cross-crystal averaging. Five total averaging/refinement cycles were performed. In cycles 2-5, major errors were found in the C222(1) model and corrected, and the rest of the macromolecule was built (residues 37-384). The final C222(1) crystal structure consists of the biologically relevant molecule, 79 waters, and one putative chloride ion, and has an R-factor of 22.46% and a free-R factor of 28.22% using data from 30 Å-2.7 Å.

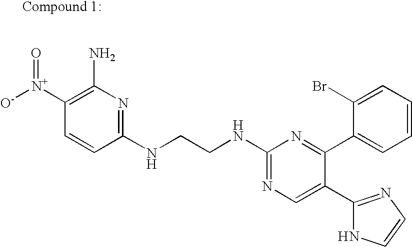

Following the completion of the refinement of C222(1) crystal structure, the resulting model and molecular replacement were then used to determine the position and structure of the molecule in the two soaked-compound C2 crystals and the P222(1) crystal. The current best GSK3β structure is from a P222(1) crystal form obtained during a soak with Compound 2 (illustrated below) which diffracts to 2.2 Å. This structure consists of two copies of the biologically relevant macromolecule in the asymmetric unit, 336 waters, 6 glycerols; this structure has an R-factor of 26.2% and free-R factor of 30.8% using data from 30 Å-2.2 Å. Compound 2 has not reached high enough occupancy in the active site to be observed.

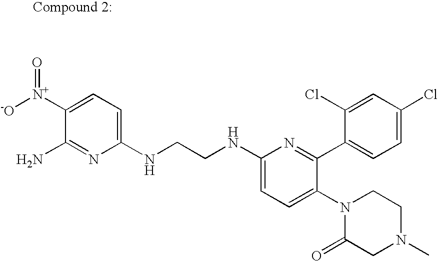

This approach was used to determine the structure due to the limiting nature of available data collection facilities at the time. More traditional methods such as single/multiple isomorphous replacement or MAD phasing by themselves or in conjunction with molecular replacement could have been used equally as well. A description of these methods and other crystallographic principles can be found in B. K. Shoichet and D. E. Bussiere, The role of macromolecular crystallography and structure for drug discovery: advances and caveats. Current Opinion in Drug Discovery & Development 2000; 3(4): 408-422.

While the preferred embodiment of the invention has been illustrated and described, it will be appreciated that various changes can be made therein without departing from the spirit and scope of the invention.

The embodiments of the invention in which an exclusive property or privilege is claimed are defined as follows:

1. A method for designing ligands that bind to a GSK3 protein, comprising:
(a) providing a purified GSK3 protein;
(b) crystallizing the purified GSK3 protein to provide a crystallized GSK3 protein having biological activity;
(c) resolving the structure of the crystallized GSK3 protein using x-ray crystallography to obtain data suitable for three-dimensional structure determination of the GSK3 protein;
(d) applying the data generated from resolving the structure of the crystallized GSK3 protein to a computer algorithm to generate a model of the GSK3 protein suitable for use in designing ligands that will bind to the GSK3 protein active site; and
(e) applying an iterative process whereby molecular structures are applied to the computer generated model to identify GSK3 binding ligands.

2. The method of claim 1, wherein the protein comprises the atomic coordinates set forth in Table 2.

3. The method of claim 1, wherein the protein comprises the amino acid sequence set forth in SEQ ID NO: 1 or an active mutant or variant thereof.

4. A method for crystallizing a human glycogen synthase kinase 3 (GSK3) protein, comprising:
(a) providing a purified GSK3 protein; and
(b) crystallizing the purified GSK3 protein to provide a crystallized GSK3 protein having biological activity, wherein the protein is crystallized from a solution comprising from about 10 to about 14 percent by weight polyethylene glycol, from about 9 to about 13 percent by weight 2-methyl-2,4-pentanediol, and from about 18 to about 22 percent by weight glycerol, and wherein the crystallized GSK3 protein is resolvable using x-ray crystallography to obtain x-ray patterns suitable for three-dimensional structure determination of the GSK3 protein.

5. The method of claim 4, wherein crystallizing the GSK3 protein comprises crystallizing by a hanging drop vapor diffusion method.

6. The method of claim 4, wherein the protein comprises the atomic coordinates set forth in Table 2.

7. The method of claim 4, wherein the protein comprises the amino acid sequence set forth in SEQ ID NO: 1 or an active mutant or variant thereof.

8. A crystallized GSK3 protein provided by the method of claim 4.
